(19) United States
(12) Patent Application Publication
(10) Pub. No.: US 2024/0279236 A1
(43) Pub. Date: Aug. 22, 2024

(54) COMPOUNDS AND METHODS OF TREATING TUBERCULOSIS

(71) Applicants: Fimbrion Therapeutics, Inc., St. Louis, MO (US); Washington University, St. Louis, MO (US); Saint Louis University, St. Louis, MO (US)

(72) Inventors: James Janetka, St. Louis, MO (US); Laurel Mydock-McGrane, St. Louis, MO (US); Keith Haynes, St. Louis, MO (US); Marvin J. Meyers, St. Louis, MO (US)

(73) Assignees: Fimbrion Therapeutics, Inc., St. Louis, MO (US); Washington University, St. Louis, MO (US); Saint Louis University, St. Louis, MO (US)

(21) Appl. No.: 18/559,471

(22) PCT Filed: May 9, 2022
(86) PCT No.: PCT/US2022/028372

§ 371 Date: Nov. 7, 2023

Related U.S. Application Data
(60) Provisional application No. 63/185,839, filed on May 7, 2021.

(51) Int. Cl. C07D 495/04; A61K 31/519; A61K 31/5377; A61K 31/541; A61K 31/695; A61K 45/06; A61P 31/06; C07D 513/04; C07D 519/00; C07F 7/08

(52) U.S. Cl. CPC C07D 495/04; A61K 31/519; A61K 31/5377; A61K 31/541; A61K 31/695; A61K 45/06; A61P 31/06; C07D 513/04; C07D 519/00; C07F 7/0812

(57) ABSTRACT

The present disclosure relates to various compounds and compositions which are useful for the treatment of tuberculosis and other diseases such as infections caused by Mycobacterium tuberculosis. The present disclosure also relates to various methods of using these compounds and compositions to treat tuberculosis and other diseases such as infections caused by Mycobacterium tuberculosis. Further, the present disclosure relates to processes of preparing these compounds and compositions.

### FIELD OF INVENTION

The present disclosure relates to various compounds and compositions that are useful for the treatment of tuberculosis and other diseases such as infections caused by Mycobacterium tuberculosis. The present disclosure also relates to various methods of using these compounds and compositions to treat tuberculosis and other diseases such as infections caused by Mycobacterium tuberculosis. Further, the present disclosure relates to processes of preparing these compounds and compositions.

### BACKGROUND OF INVENTION

Antibiotic resistant bacterial infections are a dangerous, worldwide health problem that requires costly and lengthy therapies that in many cases are ultimately ineffective. Infection with Mycobacterium tuberculosis (Mtb) results in over 10 million new cases of tuberculosis (TB) and 1.4 million deaths annually (World Health Organization Global Tuberculosis Report, 2020). A robust antibacterial defense usually controls primary Mtb infection by reducing bacterial numbers to uncultivable levels (Medlar, “The behavior of pulmonary tuberculous lesions; a pathological study,” Am. Rev. Tuberc., 71:1-244, 1955) but is often unable to eradicate the pathogen, resulting in a large population of latently-infected individuals that may reactivate the infection later in life. In addition to its ability to resist elimination by host immunity, Mtb infection is only slowly sterilized by antibiotic treatment. Patients that are latently infected with Mtb require 3-9 months of antibiotic therapy to prevent reactivation of infection, despite low bacterial burdens. To achieve clinical cure in greater than 90% of patients with active TB, multidrug antibiotic therapy for 6 months is required. Because of the long courses of antibiotic therapy, incomplete therapy is common and has resulted in the rise of multidrug-resistant (MDR) TB cases that are resistant to at least the two frontline antibiotics used to treat TB, isoniazid (INH) and rifampicin (RIF). MDR-TB constituted 3.3% of new TB cases in 2014 and 18% of previously treated TB cases, with rates of rifampicin-resistant and MDR-TB combined estimated to be as high as 53% of all TB cases in some countries (World Health Organization Global Tuberculosis Report, 2020). Furthermore, extensively drug resistant TB (XDR-TB), additionally resistant to a fluoroquinolone and an injectable antibiotic, has now been isolated in 123 countries throughout the world, including the US. Both MDR-TB and XDR-TB are extremely difficult to treat, with clinical cure rates less than 50% despite lengthy 18 to 24 month-long treatment regimens.

This rise in drug resistance and scarcity of drugs in the pipeline has made it clear that society is not equipped to successfully battle the TB epidemic. The inadequacies of present TB therapies demand the discovery of new agents with unique mechanisms of action to treat Mtb infection. The successful development of bedaquiline, a diarylquinoline that targets ATP synthase, and the first anti-mycobacterial with a novel mechanism of action in over 40 years, has greatly renewed interest in developing anti-mycobacterial drugs that disrupt energy metabolism (Bald D, Villellas C, Lu P, Koul A. Targeting Energy Metabolism in Mycobacterium tuberculosis, a New Paradigm in Antimycobacterial Drug Discovery. mBio. 2017;8(2): e00272-17). Accordingly, new therapies with different mechanisms of action are needed.

### SUMMARY OF INVENTION

Various aspects of the present disclosure are directed to compounds of Formula I, or pharmaceutically acceptable salts thereof:

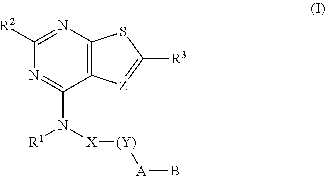

wherein:
R¹ is H, C₁-C₆ alkyl, —COOR′, —COR′, or —C(O)NHR′, where R′ is H, alkyl, cycloalkyl, or aryl;
R² is optionally substituted C₁-C₆ alkyl, optionally substituted C₃-C₆ cycloalkyl, optionally substituted C₂-C₆ alkenyl, optionally substituted aryl, optionally substituted aralkyl, optionally substituted heteroaryl, optionally substituted heteroalkyl, optionally substituted heterocycloalkyl, halo, haloalkyl, cyano, acyl, —C(CH₂)CH₃, —N₃, —CH₂OH, —CH₂OR⁴, —CH₂SR⁴, —OR⁴, —SR⁴, —SOR⁴, —SO₂R⁴, —CH₂NR⁵R⁶, —NR⁵R⁶, —CO₂H, —COR⁴, —CO₂R⁴, —NHCONR⁵R⁶, —C(R⁴)OH, —NHCOR⁴, or —CONR⁵R⁶;
R³ is H, C₁-C₆ alkyl, C₁-C₆ cycloalkyl, halo, or haloalkyl;
R⁴ is C₁-C₆ alkyl, C₃-C₆ cycloalkyl, haloalkyl, heteroalkyl, or aryl;
R⁵ and R⁶ are each independently H, optionally substituted C₁-C₆ alkyl, or optionally substituted C₃-C₆ cycloalkyl, or R⁵ and R⁶ form an optionally substituted C₃₋₆ cycloalkyl, optionally substituted heterocycloalkyl, optionally substituted heteroalkyl, optionally substituted aryl, or optionally substituted heteroaryl;
X is optionally substituted C₁-C₆ alkylene, optionally substituted C₃-C₆ cycloalkylene, optionally substituted C₂-C₆ alkenylene, optionally substituted C₃-C₆ cycloalkenylene, optionally substituted C₂-C₆ alkynylene, optionally substituted C₄-C₆ cycloalkynylene, optionally substituted carbonyl, —C(O)(CH)ₙ—, or —C(O)NH₂(CH)ₙ—, where n is an integer from 0 to 6;
Y is a bond, optionally substituted C₁-C₃ alkylene, optionally substituted amino, oxygen, sulfur, sulfoxide, or sulfone;
A is optionally substituted heterocycloalkyl, optionally substituted aryl, optionally substituted aralkyl, optionally substituted heteroaryl, or optionally substituted heteroaralkyl;
B is H, optionally substituted alkyl, optionally substituted cycloalkyl, optionally substituted heterocycloalkyl, haloalkyl, optionally substituted alkoxy, haloalkoxy, halo, cyano, optionally substituted aryl, optionally substituted aralkyl, optionally substituted heteroaryl, optionally substituted heteroaralkyl, optionally substituted heteroalkyl, optionally substituted arenediyl, —OCF₃, or —SF₅; and
Z is CH or N.

Further aspects of the present disclosure are directed to pharmaceutical compositions comprising a compound as described herein.

Other aspects of the present disclosure are directed to methods of treating tuberculosis in a subject in need thereof comprising administering to the subject a therapeutically effective amount of a compound as described herein or a pharmaceutical composition as described herein.

Additional methods of treatment include inhibiting QcrB, a subunit of the cytochrome bc₁-aa₃ supercomplex in Mtb, in a subject in need thereof. The method comprises administering to the subject a therapeutically effective amount of a compound of Formula I as disclosed herein.

Various aspects of the present disclosure are directed to methods of inhibiting the replication of a Mycobacterium tuberculosis bacterium and/or inducing the death of a Mycobacterium tuberculosis bacterium comprising contacting the bacteria with an effective amount of a compound as described herein or a pharmaceutical composition as described herein.

Other objects and features will be in part apparent and in part pointed out hereinafter.

### BRIEF DESCRIPTION OF DRAWINGS

FIG. 1A presents data for select compounds where n.d.=not determined; ^{a}In vitro profile: MLM/HLM=mouse/human liver microsome, t_{1/2}=half-life, cLogP=measure of lipophilicity; hERG=human ether-a-go-go-related gene activity; ^{b}IV bolus dose (1 mg/kg): t_{1/2}=plasma half-life, Cl=clearance; ^{c}Oral (PO) dose (2 mg/kg): % F=bioavailability.

FIG. 1B presents a compound 291 dose escalation PK study.

FIG. 1C presents a compound 291 efficacy study; statistical analysis of log₁₀-transformed efficacy data by one-way ANOVA with Tukey's correction for multiple comparisons, ** indicates P<0.05, **P<0.01, ***P<0.001, ****P<0.0001.

### DETAILED DESCRIPTION OF INVENTION

As Mtb are obligate aerobes, they rely on oxidative phosphorylation within the electron transport chain (ETC) to generate ATP. This process is critical for both Mtb growth and persistence in the host, making the ETC an attractive drug target. Herein, we disclose a new family of anti-mycobacterials that target and inhibit the oxidative active site of QcrB, a subunit of the terminal electron acceptor cytochrome bc₁-quinol reductase, which is a component of the cytochrome bc₁/aa₃ proton pumping supercomplex in the ETC of Mtb.

The present disclosure relates to various compounds and compositions which are useful for the treatment of tuberculosis and other diseases such as infections caused by Mycobacterium tuberculosis. For example, the Mycobacterium tuberculosis may be a drug resistant Mycobacterium tuberculosis which is resistant to one or more of the front line antibiotic drugs such as isoniazid and rifampicin. The present disclosure also relates to various methods of using these compounds and compositions to treat tuberculosis and other diseases such as infections caused by Mycobacterium tuberculosis. Further, the present disclosure relates to processes of preparing these compounds and compositions.

### Compounds

As noted, the present disclosure comprises compounds that are useful for the treatment of tuberculosis and other diseases such as infections caused by Mycobacterium tuberculosis.

Compounds disclosed herein can comprise those of Formula I, or pharmaceutically acceptable salts thereof:

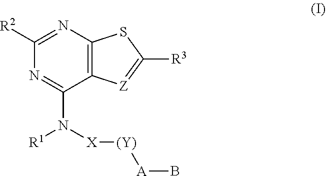

wherein:
R¹ is H, C₁-C₆ alkyl, —COOR′, —COR′, or —C(O)NHR′, where R′ is H, alkyl, cycloalkyl, or aryl;
R² is optionally substituted C₁-C₆ alkyl, optionally substituted C₃-C₆ cycloalkyl, optionally substituted C₂-C₆ alkenyl, optionally substituted aryl, optionally substituted aralkyl, optionally substituted heteroaryl, optionally substituted heteroalkyl, optionally substituted heterocycloalkyl, halo, haloalkyl, cyano, acyl, —C(CH₂)CH₃, —N₃, —CH₂OH, —CH₂OR⁴, —CH₂SR⁴, —OR⁴, —SR⁴, —SOR⁴, —SO₂R⁴, —CH₂NR⁵R⁶, —NR⁵R⁶, —CO₂H, —COR⁴, —CO₂R⁴, —NHCONR⁵R⁶, —C(R⁴)OH, —NHCOR⁴, or —CONR⁵R⁶;
R³ is H, C₁-C₆ alkyl, C₁-C₆ cycloalkyl, halo, or haloalkyl;
R⁴ is C₁-C₆ alkyl, C₃-C₆ cycloalkyl, haloalkyl, heteroalkyl, or aryl;
R⁵ and R⁶ are each independently H, optionally substituted C₁-C₆ alkyl, or optionally substituted C₃-C₆ cycloalkyl, or R⁵ and R⁶ form an optionally substituted C₃₋₆ cycloalkyl, optionally substituted heterocycloalkyl, optionally substituted heteroalkyl, optionally substituted aryl, or optionally substituted heteroaryl;
X is optionally substituted C₁-C₆ alkylene, optionally substituted C₃-C₆ cycloalkylene, optionally substituted C₂-C₆ alkenylene, optionally substituted C₃-C₆ cycloalkenylene, optionally substituted C₂-C₆ alkynylene, optionally substituted C₄-C₆ cycloalkynylene, optionally substituted carbonyl, —C(O)(CH)ₙ—, or —C(O)NH₂(CH)ₙ—, where n is an integer from 0 to 6;
Y is a bond, optionally substituted C₁-C₃ alkylene, optionally substituted amino, oxygen, sulfur, sulfoxide, or sulfone;
A is optionally substituted heterocycloalkyl, optionally substituted aryl, optionally substituted aralkyl, optionally substituted heteroaryl, or optionally substituted heteroaralkyl;
B is H, optionally substituted alkyl, optionally substituted cycloalkyl, optionally substituted heterocycloalkyl, haloalkyl, optionally substituted alkoxy, haloalkoxy, halo, cyano, optionally substituted aryl, optionally substituted aralkyl, optionally substituted heteroaryl, optionally substituted heteroaralkyl, optionally substituted heteroalkyl, optionally substituted arenediyl, —OCF₃, or —SF₅; and
Z is CH or N.

Compounds of the present disclosure include those of Formula I, or a pharmaceutically acceptable salt thereof:

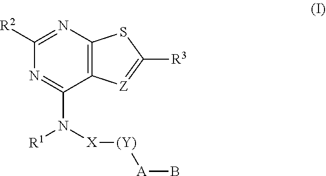

wherein:
R¹ is H, C₁-C₆ alkyl, —COOR′, —COR′, or —C(O)NHR′, where R′ is H, alkyl, cycloalkyl, or aryl;
R² is optionally substituted C₁-C₆ alkyl, optionally substituted C₃-C₆ cycloalkyl, optionally substituted C₂-C₆ alkenyl, optionally substituted aryl, optionally substituted aralkyl, optionally substituted heteroaryl, optionally substituted heteroalkyl, optionally substituted heterocycloalkyl, halo, haloalkyl, cyano, —C(CH₂)CH₃, —N₃, —CH₂OH, —CH₂OR⁴, —CH₂SR⁴, —OR⁴, —SR⁴, —SOR⁴, —SO₂R⁴, —CH₂NR⁵R⁶, —NR⁵R⁶, or —CONR⁵R⁶;
R³ is H, C₁-C₆ alkyl, C₁-C₆ cycloalkyl, halo, or haloalkyl;
R⁴ is C₁-C₆ alkyl, C₃-C₆ cycloalkyl, haloalkyl, or aryl;
R⁵ and R⁶ are each independently H, optionally substituted C₁-C₆ alkyl, or optionally substituted C₃-C₆ cycloalkyl, or R⁵ and R⁶ form an optionally substituted C₃₋₆ cycloalkyl, optionally substituted heterocycloalkyl, optionally substituted aryl, or optionally substituted heteroaryl;
X is optionally substituted C₁-C₆ alkylene, optionally substituted C₃-C₆ cycloalkylene, optionally substituted C₂-C₆ alkenylene, optionally substituted C₃-C₆ cycloalkenylene, optionally substituted C₂-C₆ alkynylene, optionally substituted C₄-C₆ cycloalkynylene, optionally substituted carbonyl, —C(O)(CH)ₙ—, or —C(O)NH₂(CH)ₙ—, where n is an integer from 0 to 6 (e.g., n is preferably 1, 2, or 3);
Y is a bond, optionally substituted C₁-C₃ alkylene, optionally substituted amino, oxygen, sulfur, sulfoxide, or sulfone;
A is optionally substituted heterocycloalkyl, optionally substituted aryl, optionally substituted aralkyl, optionally substituted heteroaryl, or optionally substituted heteroaralkyl;
B is H, optionally substituted alkyl, optionally substituted cycloalkyl, optionally substituted heterocycloalkyl, haloalkyl, optionally substituted alkoxy, haloalkoxy, halo, cyano, optionally substituted aryl, optionally substituted aralkyl, optionally substituted heteroaryl, optionally substituted heteroaralkyl, —OCF₃, or —SF₅; and
Z is CH or N.

For the compounds of Formula I, R¹ can be H.

R² can comprise a moiety comprising at least one heteroatom selected from the group consisting of N, O, and S.

R² can also be C₁-C₆ alkyl, C₃-C₆ cycloalkyl, halo, haloalkyl, aryl, optionally substituted 5- or 6-membered heteroaryl, optionally substituted 4-, 5-, or 6-membered heterocycloalkyl, —CH₂OR⁴, —CH₂SR⁴, —OR⁴, —SR⁴, —SOR⁴, —SO₂R⁴, —NR⁵R⁶, —CH₂NR⁵R⁶, —COR⁴, —CO₂R⁴, —NHCOR⁴, or —CONR⁵R⁶;

R⁴ can be C₁-C₆ alkyl or haloalkyl; and/or

R⁵ and R⁶ can each independently be H, C₁-C₆ alkyl, or C₃-C₆ cycloalkyl.

R² can also be halo, haloalkyl, aryl, 5-membered heteroaryl, 6-membered heteroaryl, C₁-C₆ alkyl-substituted 5-membered heteroaryl, C₁-C₆ alkyl-substituted 6-membered heteroaryl, halo-substituted 5-membered heteroaryl, halo-substituted 6-membered heteroaryl, 4-membered heterocycloalkyl, 5-membered heterocycloalkyl, C₁-C₆ alkyl-substituted 4-membered heterocycloalkyl, C₁-C₆ alkyl-substituted 5-membered heterocycloalkyl, halo-substituted 4-membered heterocycloalkyl, halo-substituted 5-membered heterocycloalkyl, —OR⁴, —NR⁵R⁶, —SR⁴, —COR⁴, —CO₂R⁴, —NHCOR⁴, or —CONR⁵R⁶;

R⁴ can be C₁-C₄ alkyl or C₁-C₄haloalkyl; and/or

R⁵ and R⁶ can each independently be H or C₁-C₄ alkyl.

For example, R² can be: methoxy, dimethylamino,

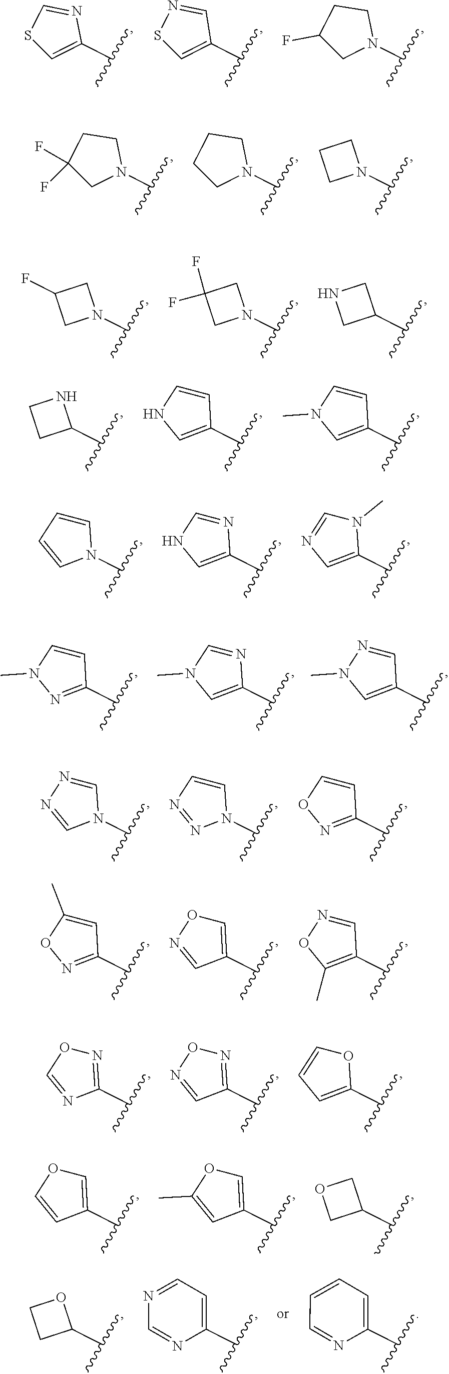

R³ can be H or C₁-C₄ alkyl. For example, R³ can be methyl.

X can be optionally substituted C₁-C₃ alkylene, optionally substituted C₃-C₅ cycloalkylene, optionally substituted C₂-C₆ alkenylene, optionally substituted C₂-C₆ alkynylene, optionally substituted carbonyl, —C(O)(CH)ₙ—, or —C(O)NH₂(CH)ₙ—, where n is an integer from 0 to 3. For example, X can be C₁-C₃ alkylene, C₃-C₅ cycloalkylene, C₂-C₆ alkenylene, C₂-C₆ alkynylene, carbonyl, —C(O)(CH)ₙ—, —C(O)NH₂(CH)ₙ—, where n is an integer from 0 to 3.

Preferably, X is —CH₂CH₂—, —CH₂CH₂CH₂—, —CH₂CHCH—, —CH₂CC—, —C(O)CH₂—, —C(O)CH₂CH₂—, —C(O)CH₂CH₂CH₂—, —C(O)NHCH₂—, —C(O)NHCH₂CH₂—,

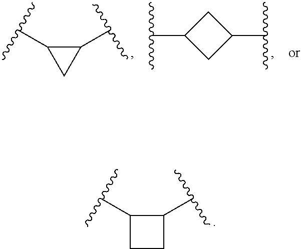

Y can be a bond. Y can also be C₁-C₃ alkylene.

A can be optionally substituted heterocycloalkyl, optionally substituted aryl, or optionally substituted heteroaryl. For example, A can be optionally substituted phenyl or an optionally substituted 5- or 6-membered heterocycloalkyl or heteroaryl comprising at least one nitrogen heteroatom.

Preferably, A is:

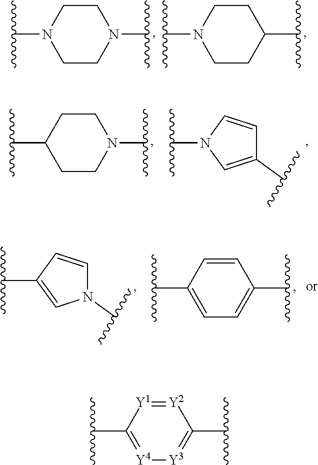

wherein Y¹, Y², Y³, and Y⁴ are each independently selected from CR⁷ or N; and R⁷ is H, alkyl, —CF₃, alkoxy, —OCF₃, halo, or cyano; Preferably, R⁷ is H, methyl, —CF₃, —OCH₃, —OCF₃, fluoro, or cyano.

B can be —OCF₃, —SF₅, optionally substituted heterocycloalkyl, optionally substituted aryl, optionally substituted aralkyl, optionally substituted heteroaryl, optionally substituted heteroalkyl, or optionally substituted heteroaralkyl. For example, B can be optionally substituted heterocycloalkyl, optionally substituted aryl, optionally substituted aralkyl, optionally substituted heteroaryl, or optionally substituted heteroaralkyl. B can be optionally substituted phenyl, optionally substituted 5- or 6-membered heterocycloalkyl comprising at least one nitrogen heteroatom, or an optionally substituted 5- or 6-membered heteroaryl comprising at least one nitrogen heteroatom.

Preferably, B is:

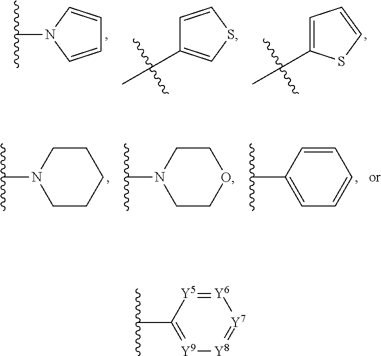

wherein Y⁵, Y⁶, Y⁷, Y⁸, and Y⁹ are each independently selected from CR⁸ or N and Yⁿ and Yⁿ⁺¹ can form a fused ring (e.g., Y⁵ and Y⁶, Y⁶ and Y⁷, Y⁷ and Y⁸, and Y⁸ and Y⁹); and R⁸ is H, optionally substituted alkyl, optionally substituted C₃-C₅ cycloalkyl, cyclopropyl, cyclopropoxy, —CF₃, optionally substituted alkoxy, —OCF₃, —SF₅, halo, cyano, or —NR⁹R¹⁰; and R⁹ and R¹⁰ are each independently H or C₁-C₆ alkyl.

A can be

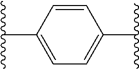 [SMILES: *c1ccc(*)cc1]

### and B can be:

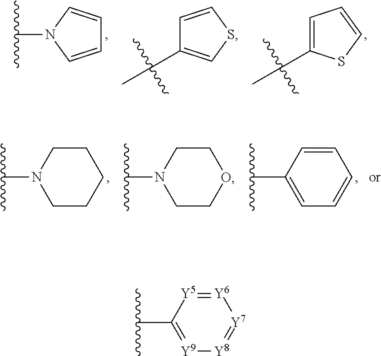

wherein Y⁵, Y⁶, Y⁷, Y⁸, and Y⁹ are each independently selected from CR⁸ or N and Yⁿ and Yⁿ⁺¹ can form a fused ring (e.g., Y⁵ and Y⁶, Y⁶ and Y⁷, Y⁷ and Y⁸, and Y⁸ and Y⁹); and R⁸ is H, optionally substituted alkyl, optionally substituted C₃-C₅ cycloalkyl, cyclopropyl, cyclopropoxy, —CF₃, optionally substituted alkoxy, —OCF₃, —SF₅, halo, cyano, or —NR⁹R¹⁰; and R⁹ and R¹⁰ are each independently H or C₁-C₆ alkyl.

B can be:

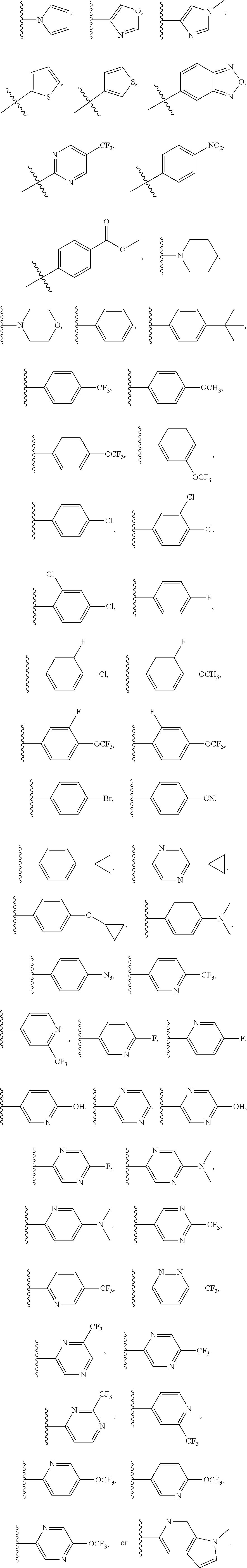

Z can be N.

Z can also be CH.

When Z is CH, then R² can comprise a moiety comprising at least one heteroatom selected from the group consisting of N, O, and S.

When Z is CH, then B is not hydrogen in some compounds.

When Z is CH, then R² can be optionally substituted C₃-C₆ cycloalkyl, optionally substituted C₂-C₆ alkenyl, optionally substituted aryl, optionally substituted aralkyl, optionally substituted heteroaryl, optionally substituted heteroalkyl, optionally substituted heterocycloalkyl, halo, haloalkyl, cyano, —C(CH₂)CH₃, —N₃, —CH₂OH, —CH₂OR⁴, —CH₂SR⁴, —OR⁴, —SR⁴, —SOR⁴, —SO₂R⁴, —CH₂NR⁵R⁶, —NR⁵R⁶, —CO₂H, —COR⁴, —CO₂R⁴, —NHCONR⁵R⁶, —C(R⁴)OH, —NHCOR⁴, or —CONR⁵R⁶, where R⁴, R⁵, and R⁶ are as defined herein.

When Z is CH, then R² can be halo, aryl, 5-membered heteroaryl, 6-membered heteroaryl, C₁-C₆ alkyl-substituted 5-membered heteroaryl, C₁-C₆ alkyl-substituted 6-membered heteroaryl, halo-substituted 5-membered heteroaryl, halo-substituted 6-membered heteroaryl, 4-membered heterocycloalkyl, 5-membered heterocycloalkyl, C₁-C₆ alkyl-substituted 4-membered heterocycloalkyl, C₁-C₆ alkyl-substituted 5-membered heterocycloalkyl, halo-substituted 4-membered heterocycloalkyl, halo-substituted 5-membered heterocycloalkyl, —OR⁴, —NR⁵R⁶, —SR⁴, —CO₂H, —COR⁴, —CO₂R⁴, —NHCONR⁵R⁶, —C(R⁴)OH, —NHCOR⁴, or —CONR⁵R⁶, where R⁴, R⁵, and R⁶ are as defined herein.

When Z is CH, then R² is not methyl, ethyl, trifluoromethyl, or perfluoroethyl in some compounds.

Compounds can also have the structure of Formula IA, or a pharmaceutically acceptable salt thereof:

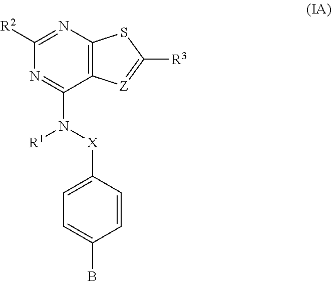

wherein R¹ is H or C₁-C₆ alkyl; R² is optionally substituted C₂-C₆ alkenyl, optionally substituted heteroaryl, optionally substituted heteroalkyl, —OR⁴, —SR⁴, or —NR⁵R⁶; R³ is H, C₁-C₆ alkyl, or haloalkyl; preferably, R³ is CH₃, C₁, or CF₃; R⁴ is C₁-C₆ alkyl; R⁵ and R⁶ are each independently H or optionally substituted C₁-C₆ alkyl; X is optionally substituted C₁-C₆ alkylene; B is

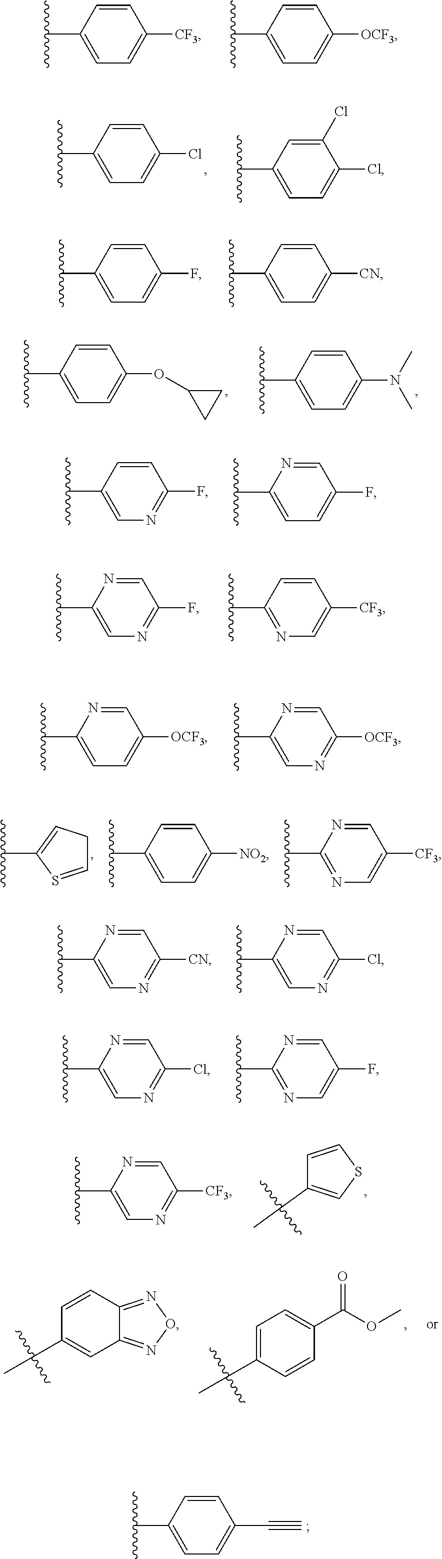

### and Z is CH or N.

The compounds of Formula IA can also have a structure where R¹ is H.

The compounds of Formula IA can have a structure wherein R² is furyl, isoxazolyl, thiophenyl, pyrrolyl, —OR⁴, —SR⁴, or —NR⁵R⁶; wherein R⁴ is C₁-C₃ alkyl, and R⁵ and R⁶ are each independently C₁-C₃ alkyl.

The compounds of Formula IA can further have a structure wherein R⁴ is independently methyl and R⁵ and R⁶ is methyl.

The compounds of Formula IA can have a structure wherein R³ is methyl.

The compounds of Formula IA can have a structure wherein X is ethylene, propylene, butylene, or pentylene.

The compounds of Formula IA can have a structure wherein X is propylene.

The compounds of Formula IA can also have a structure wherein B is

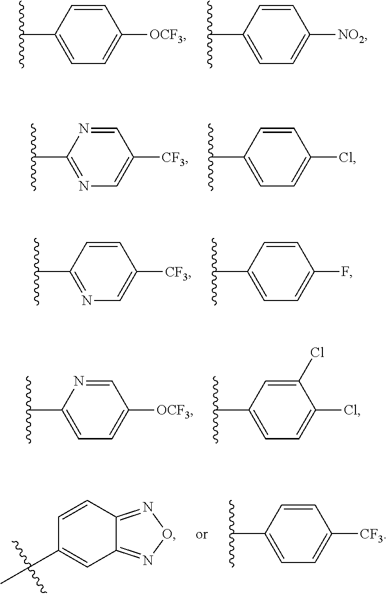

The compound can exhibit an IC₅₀ against Mycobacterium tuberculosis at about 200 nM or less.

The compound can be selected from any of the compounds listed below:

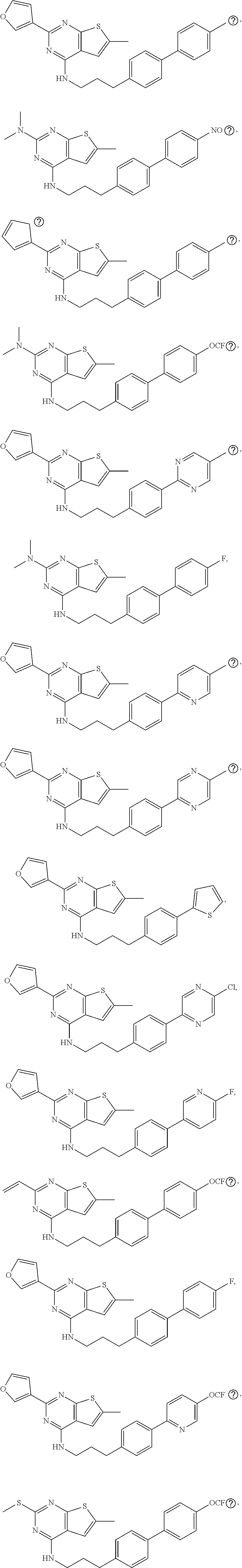

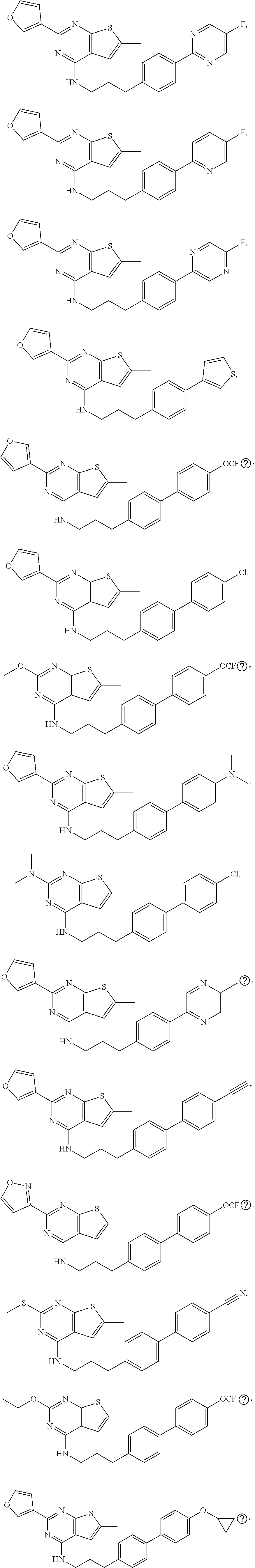

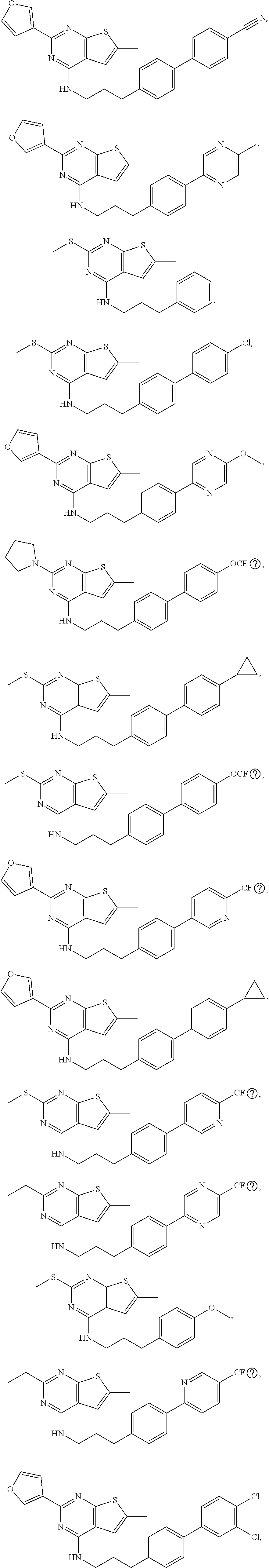

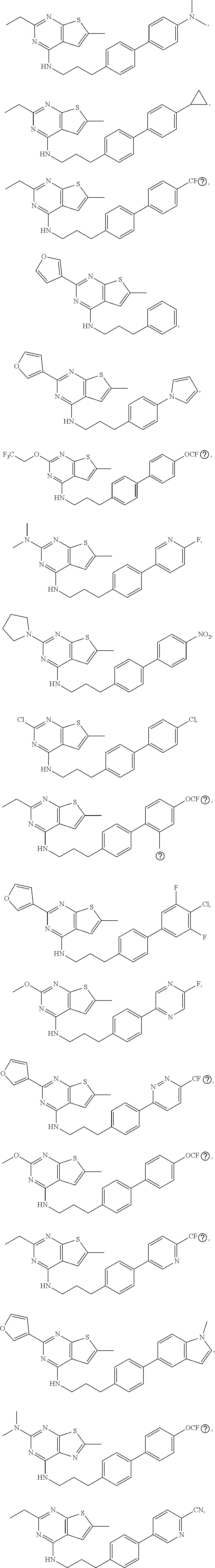

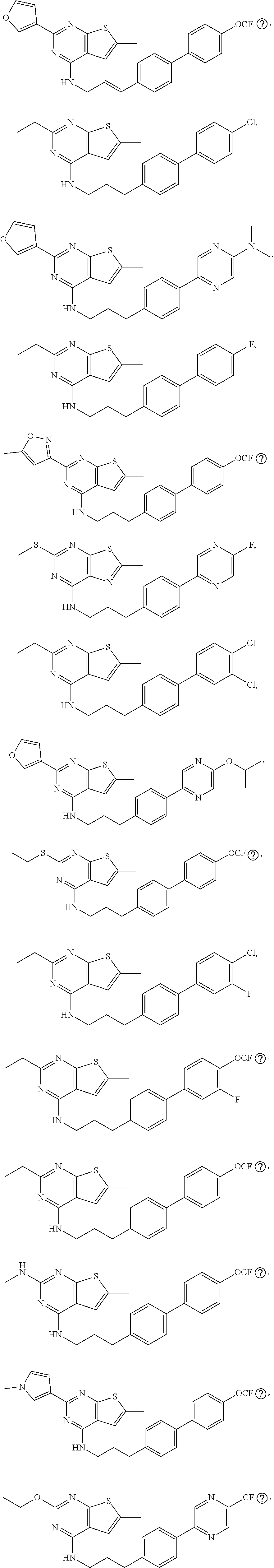

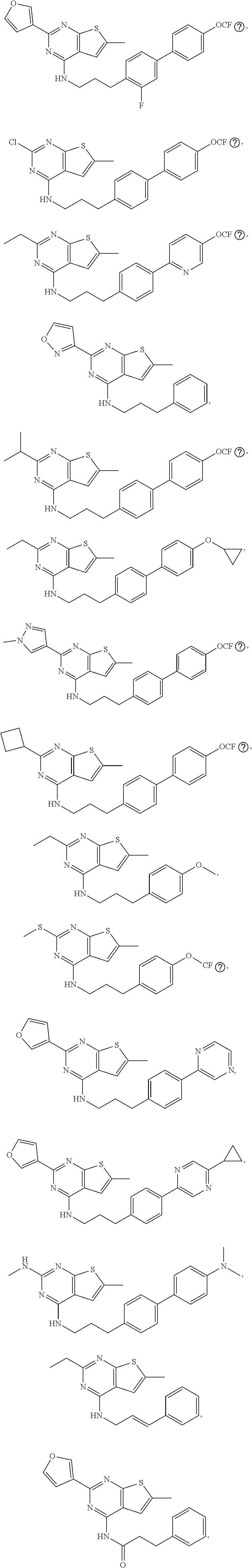

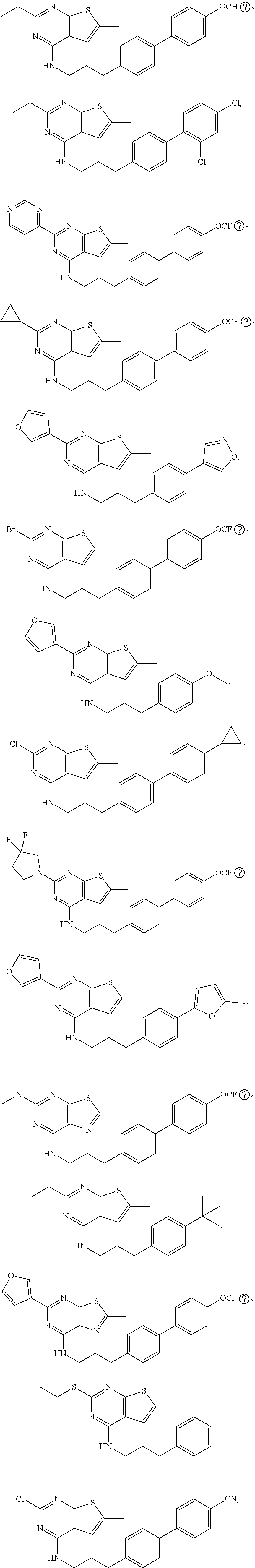

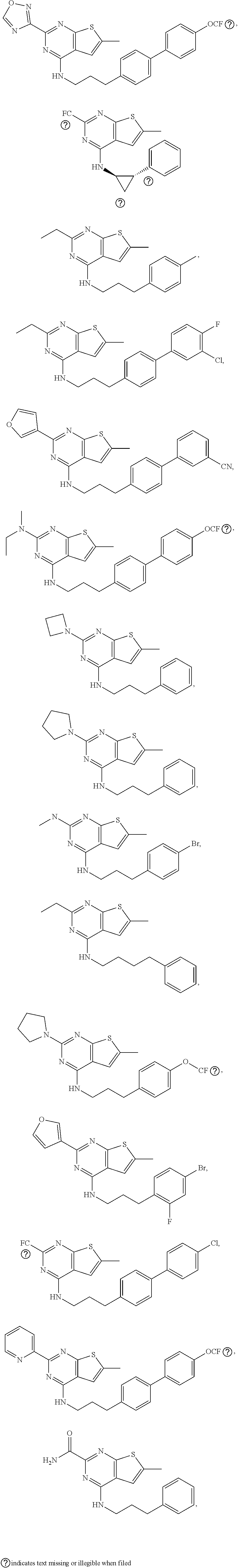

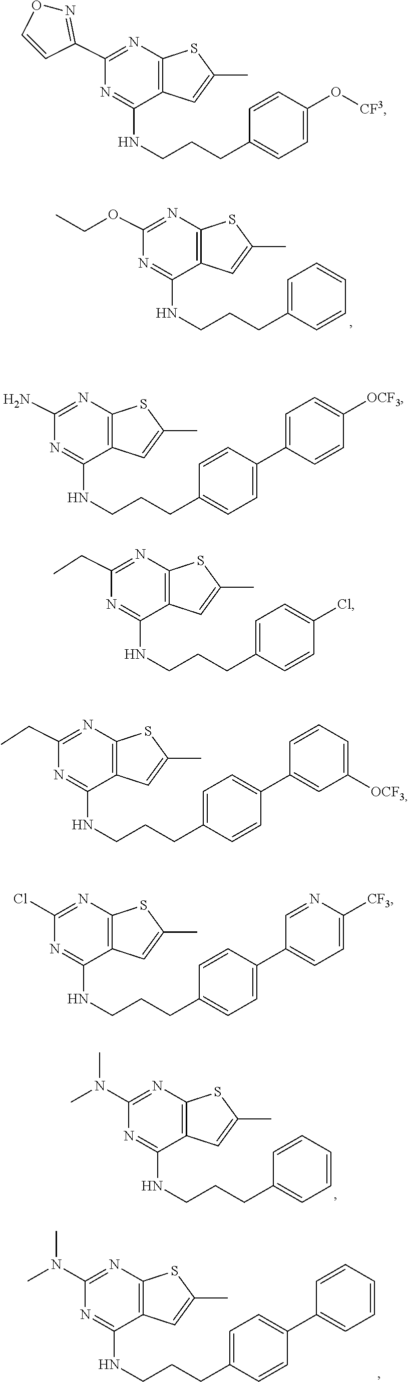

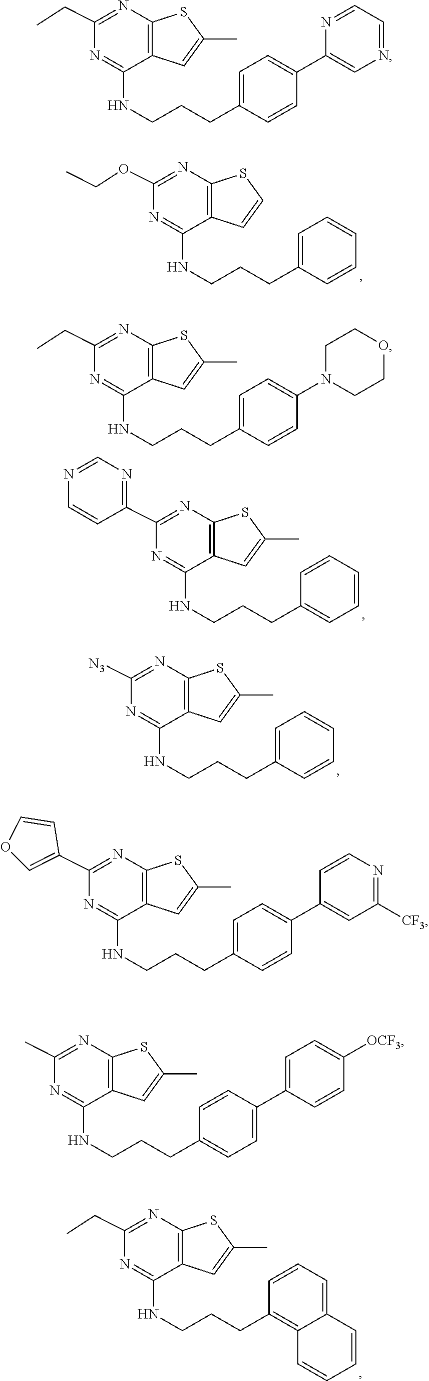

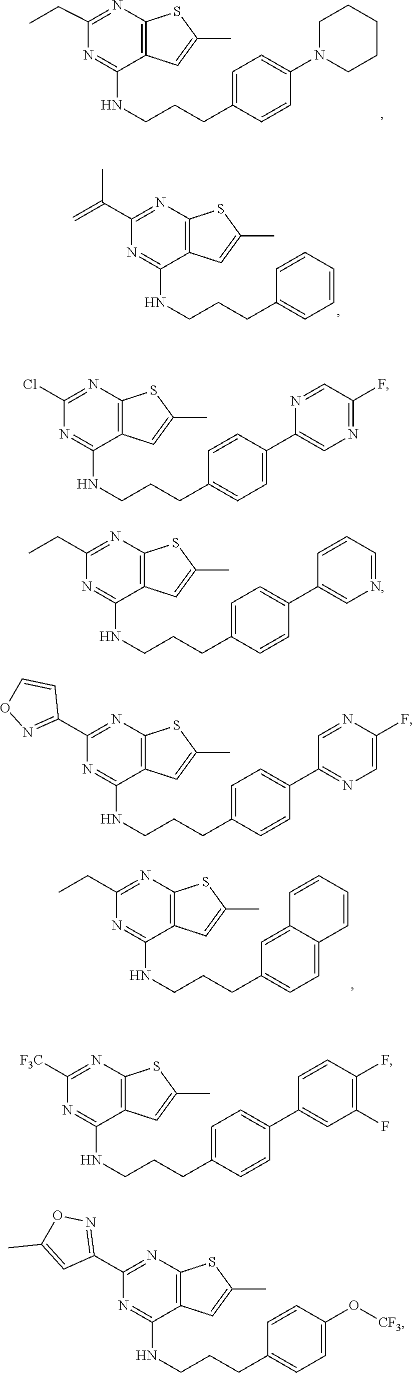

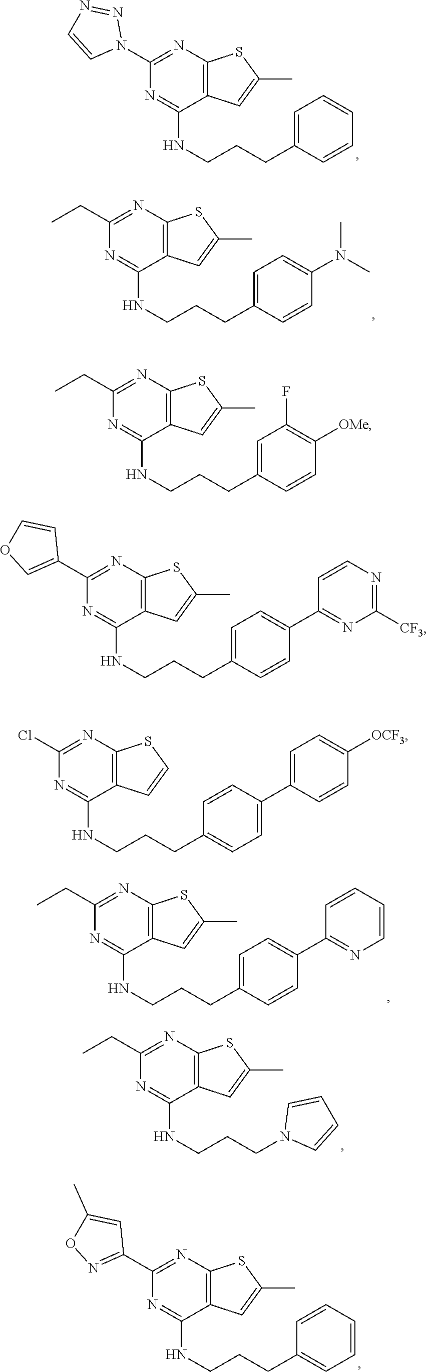

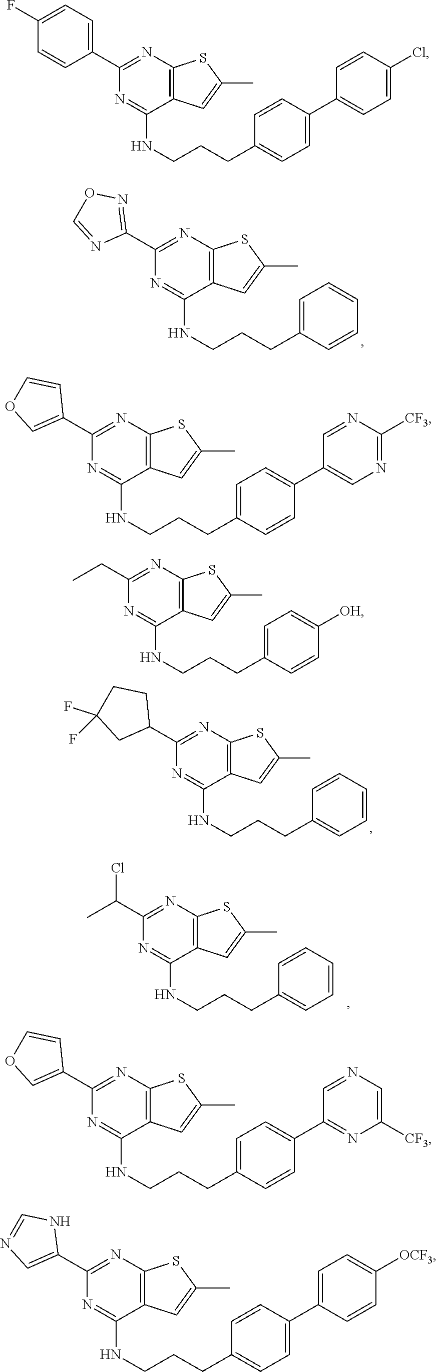

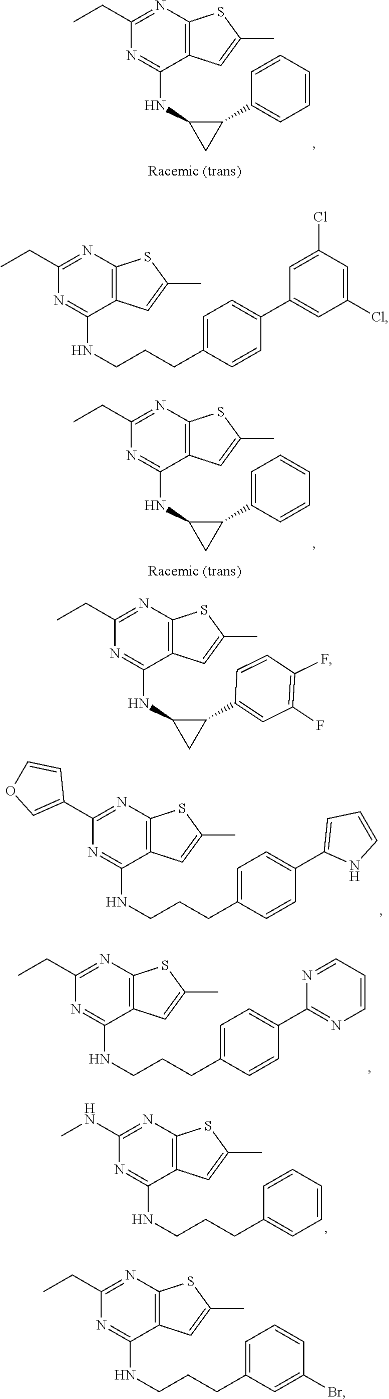

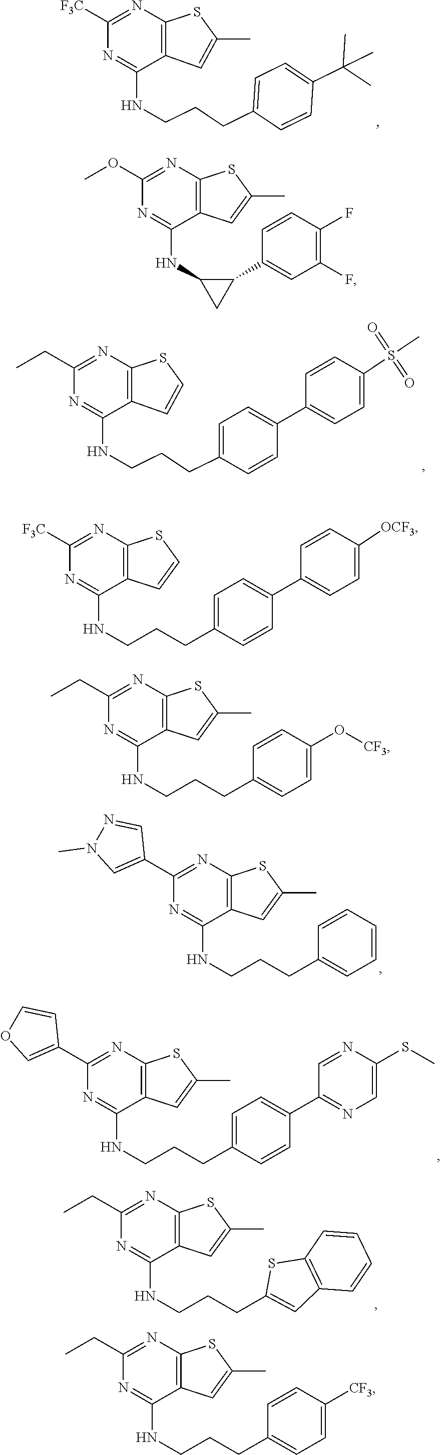

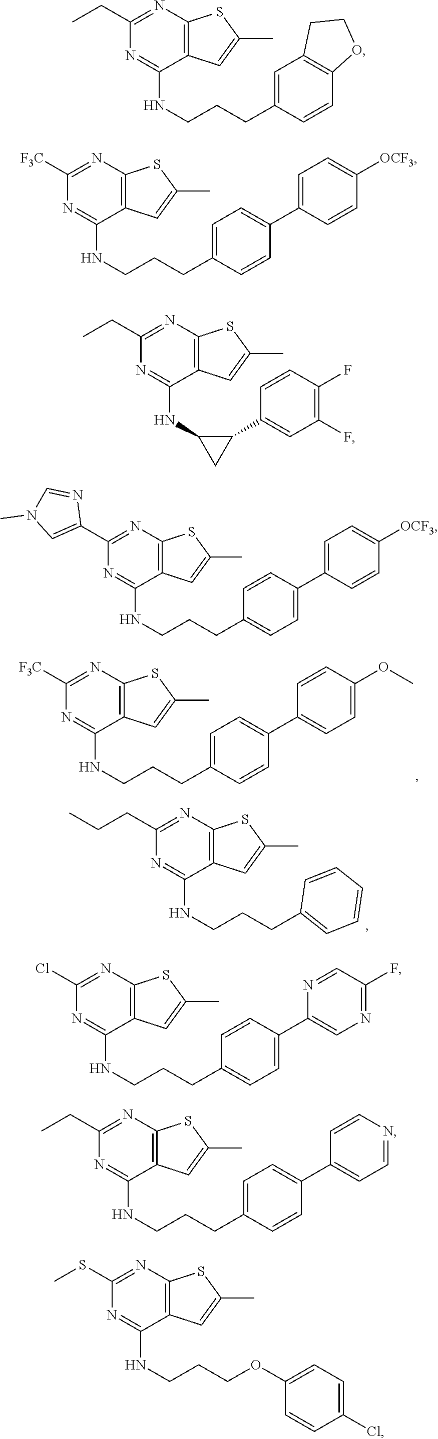

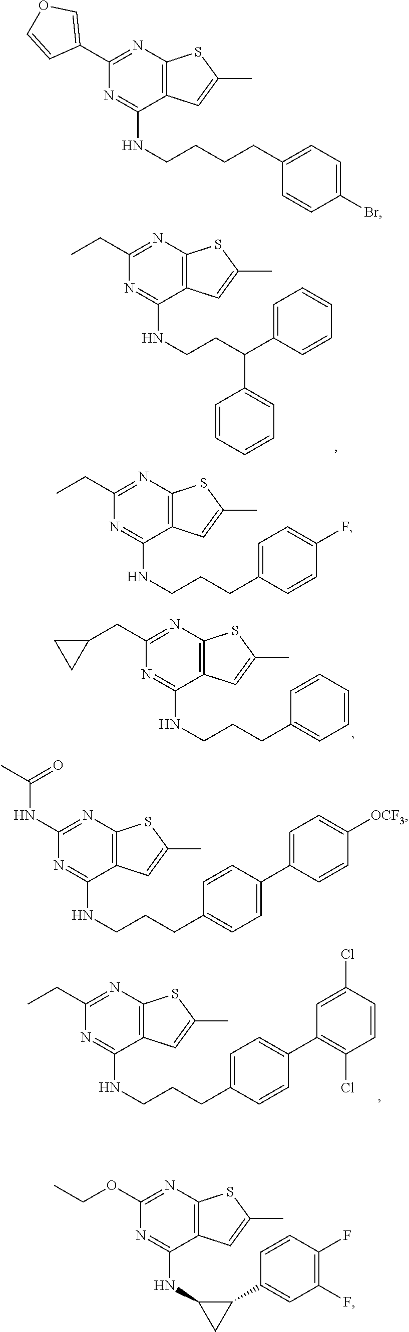

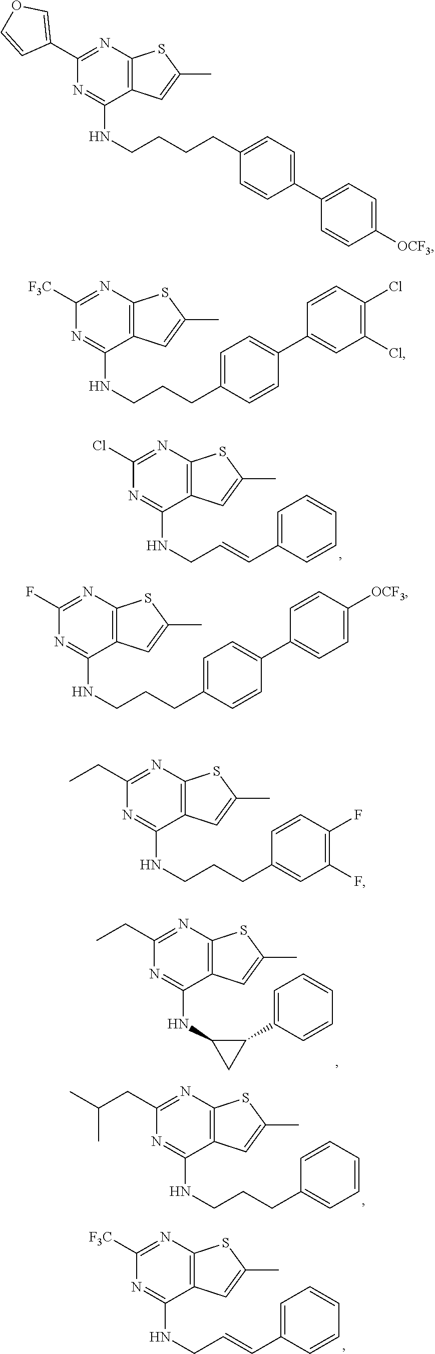

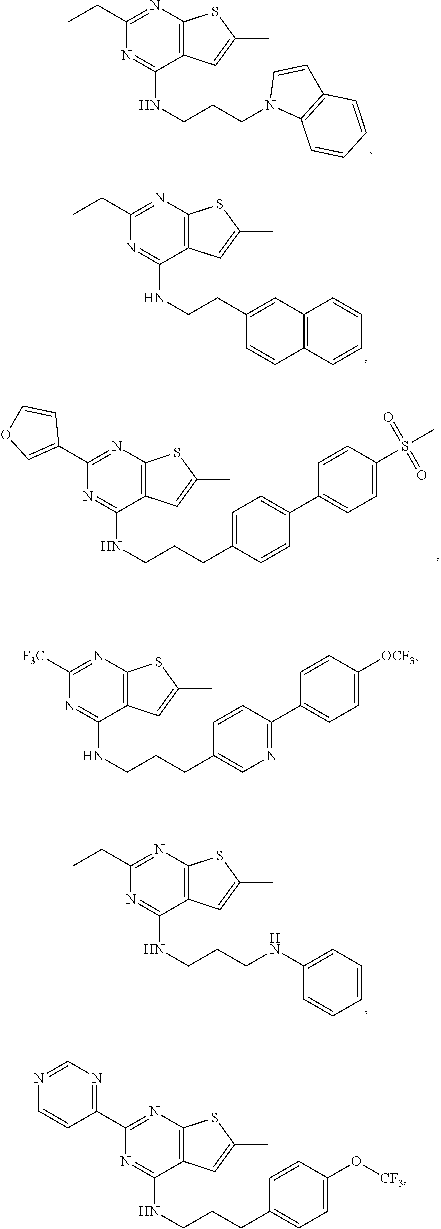

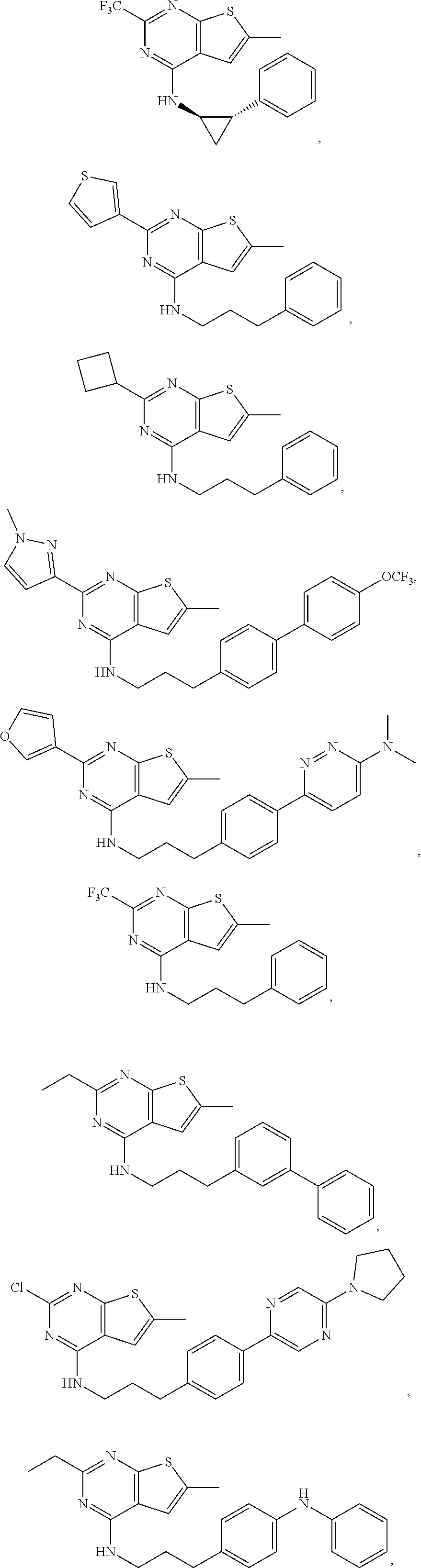

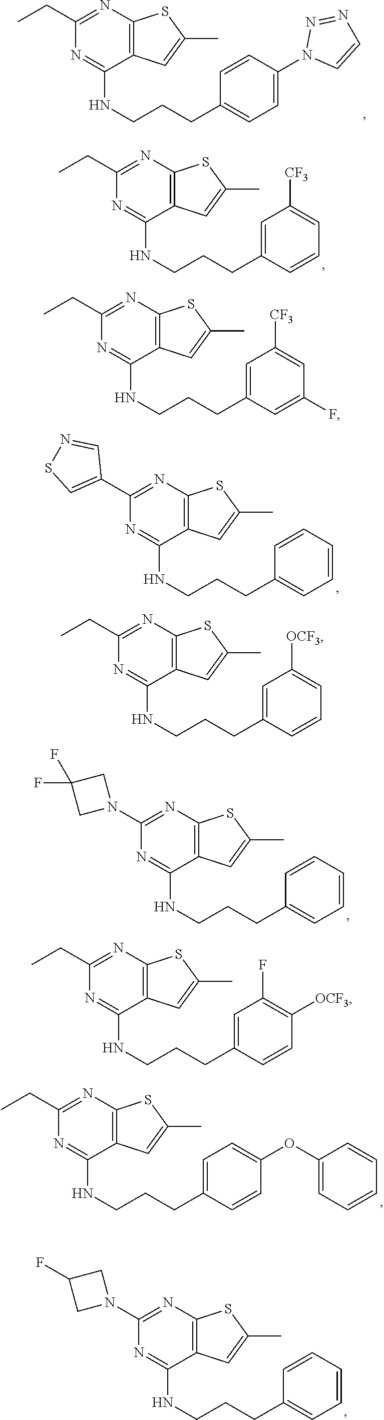

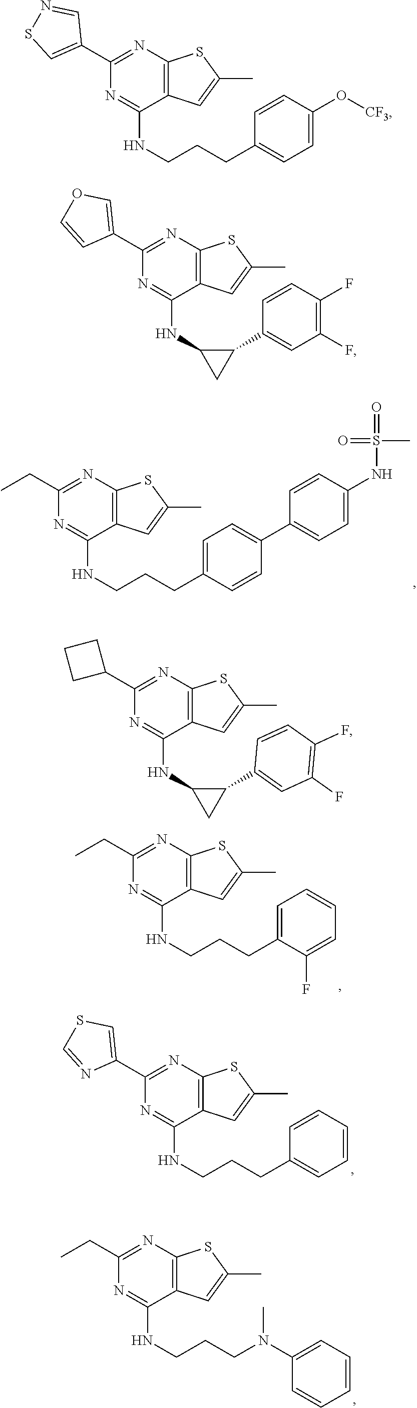

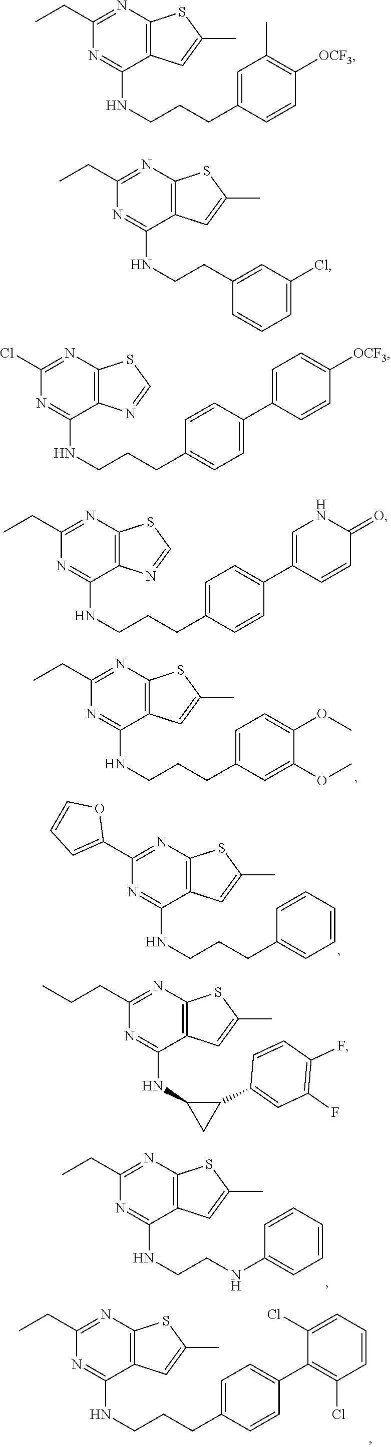

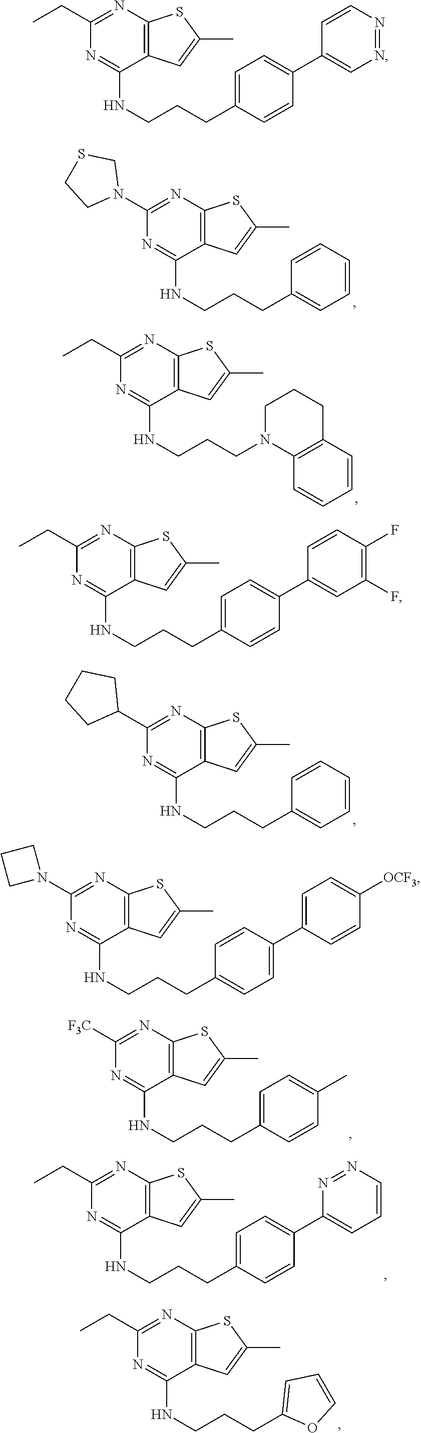

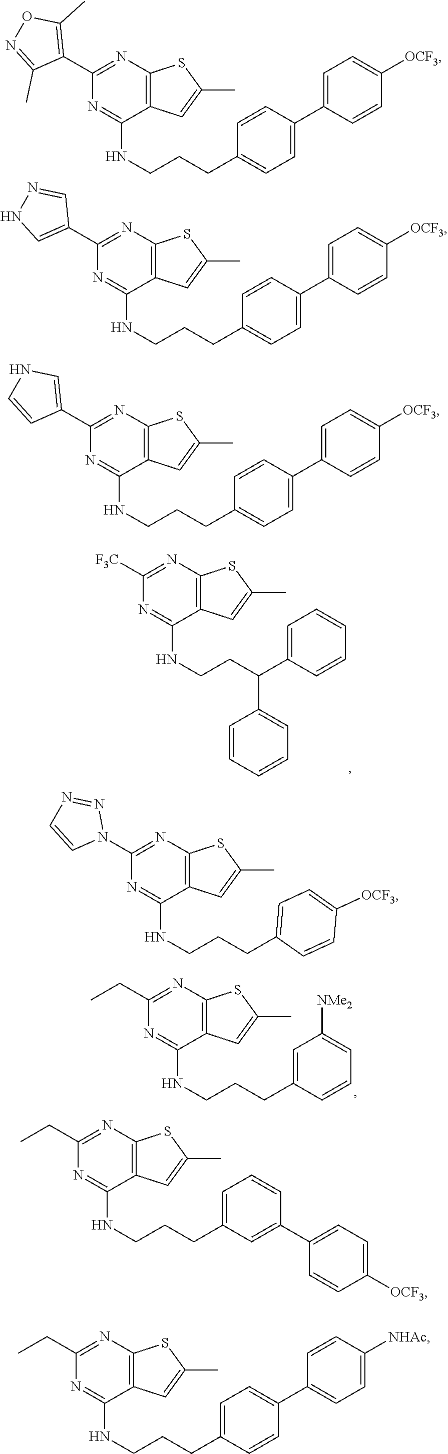

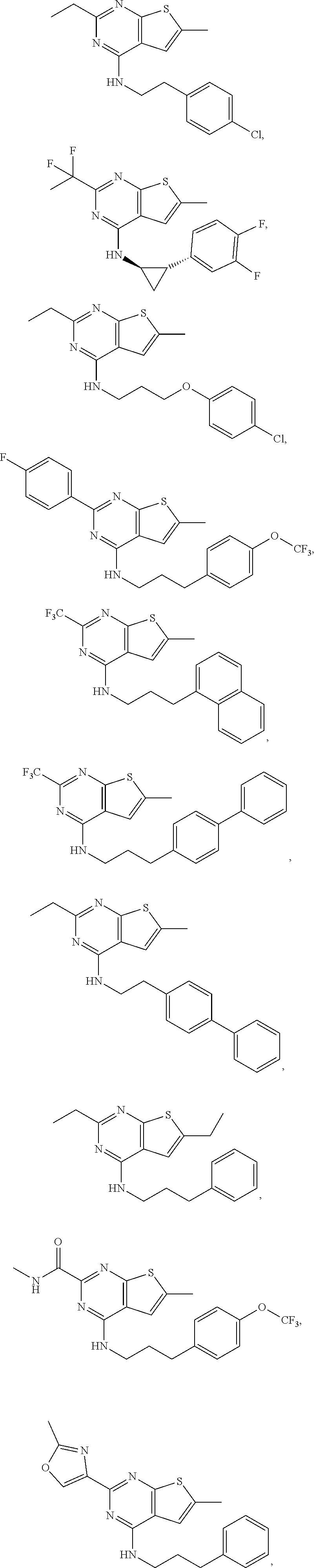

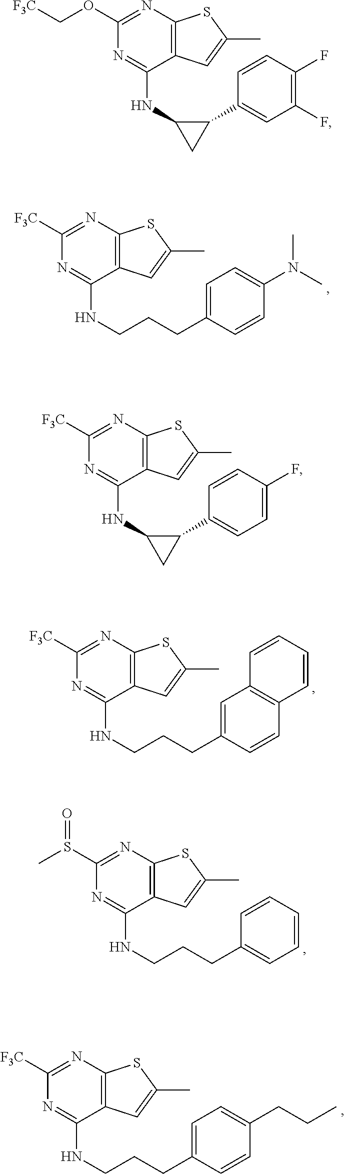

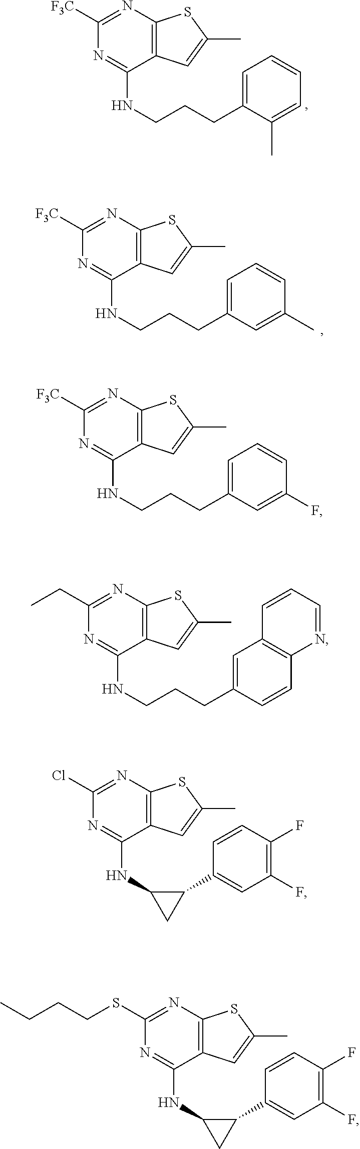

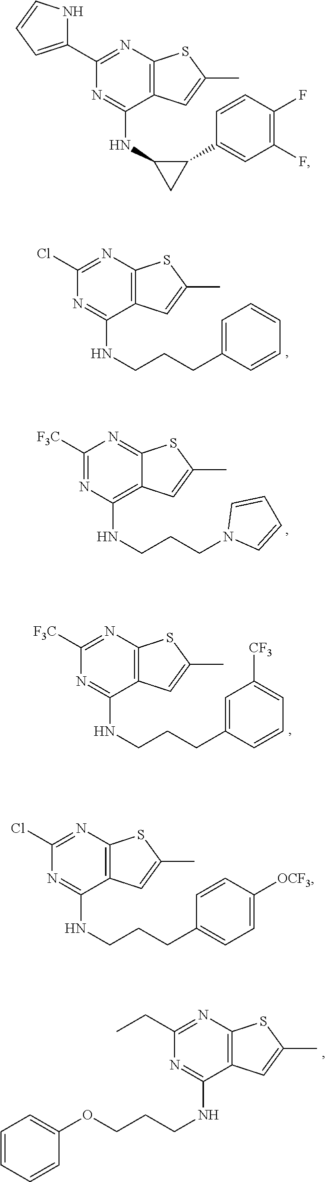

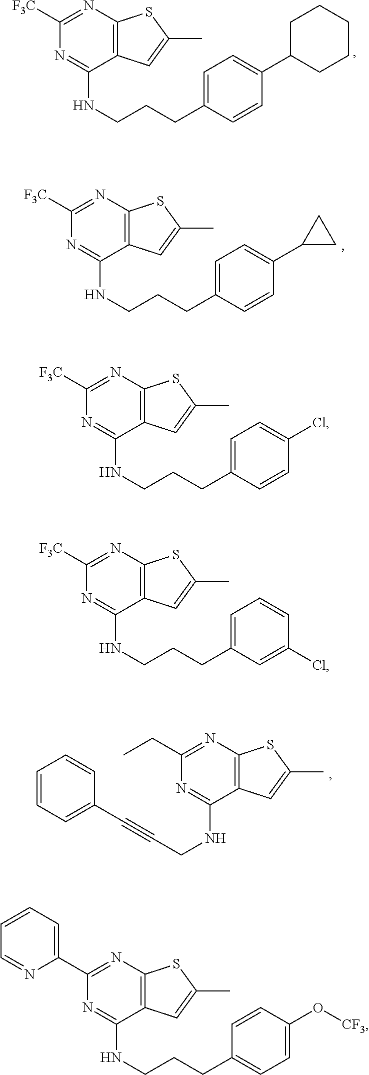 [SMILES: Cc1cc2c(NCCCc3ccc(C4CCCCC4)cc3)nc(C(F)(F)F)nc2s1 ; Cc1cc2c(NCCCc3ccc(C4CC4)cc3)nc(C(F)(F)F)nc2s1 ; Cc1cc2c(NCCCc3ccc(Cl)cc3)nc(C(F)(F)F)nc2s1 ; Cc1cc2c(NCCCc3cccc(Cl)c3)nc(C(F)(F)F)nc2s1 ; CCc1nc(NCC#Cc2ccccc2)c2cc(C)sc2n1 ; Cc1cc2c(NCCCc3ccc(OC(F)(F)F)cc3)nc(-c3ccccn3)nc2s1]

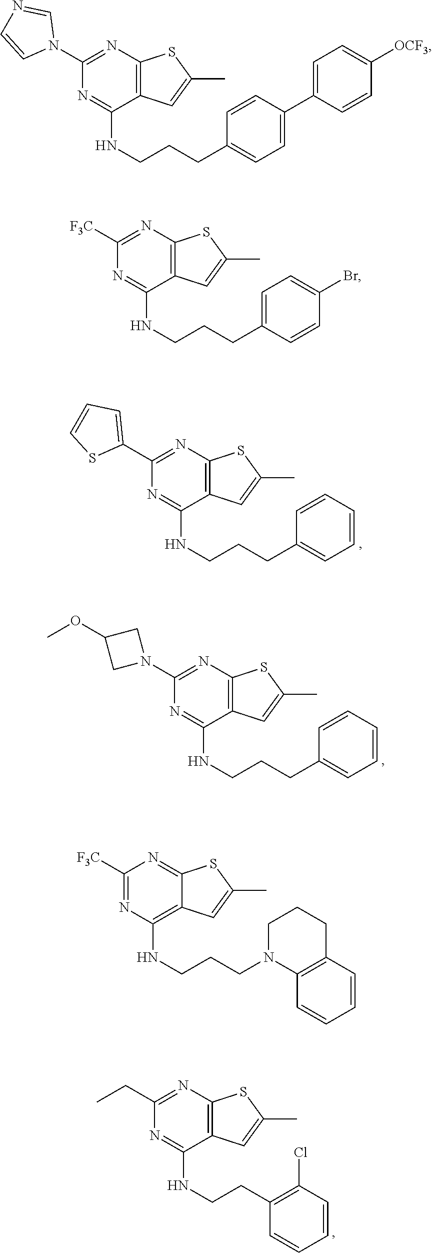 [SMILES: Cc1cc2c(NCCCc3ccc(-c4ccc(OC(F)(F)F)cc4)cc3)nc(-n3ccnc3)nc2s1 ; Cc1cc2c(NCCCc3ccc(Br)cc3)nc(C(F)(F)F)nc2s1 ; Cc1cc2c(NCCCc3ccccc3)nc(-c3cccs3)nc2s1 ; COC1CN(c2nc(NCCCc3ccccc3)c3cc(C)sc3n2)C1 ; Cc1cc2c(NCCCN3CCCc4ccccc43)nc(C(F)(F)F)nc2s1 ; CCc1nc(NCCc2ccccc2Cl)c2cc(C)sc2n1]

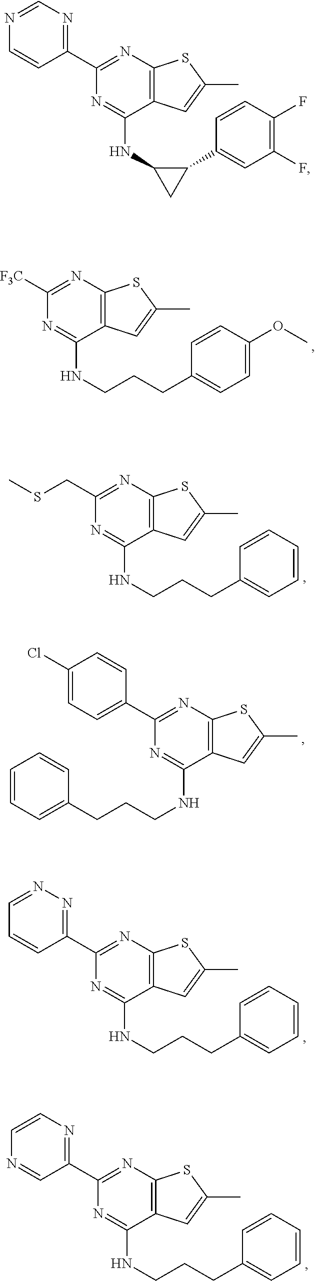

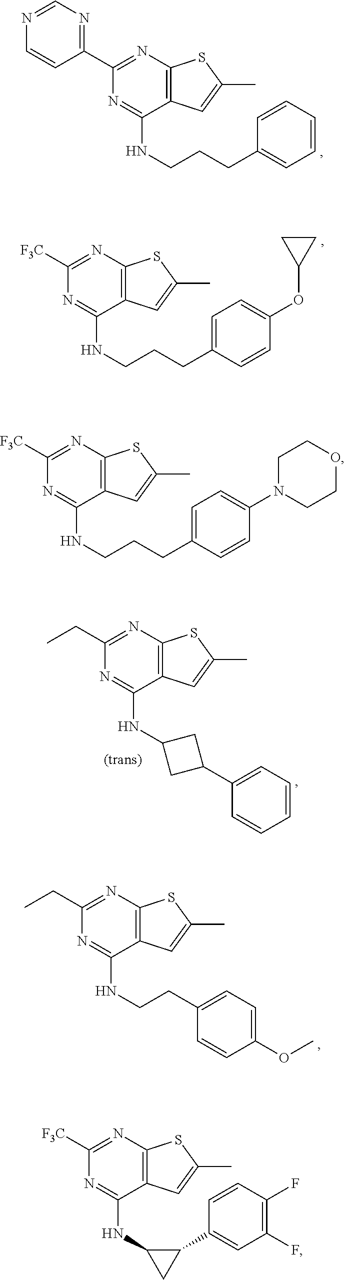 [SMILES: Cc1cc2c(NCCCc3ccccc3)nc(-c3ccncn3)nc2s1 ; Cc1cc2c(NCCCc3ccc(OC4CC4)cc3)nc(C(F)(F)F)nc2s1 ; Cc1cc2c(NCCCc3ccc(N4CCOCC4)cc3)nc(C(F)(F)F)nc2s1 ; CCc1nc(NC2CC(c3ccccc3)C2)c2cc(C)sc2n1 ; CCc1nc(NCCc2ccc(OC)cc2)c2cc(C)sc2n1 ; Cc1cc2c(N[C@@H]3C[C@H]3c3ccc(F)c(F)c3)nc(C(F)(F)F)nc2s1]

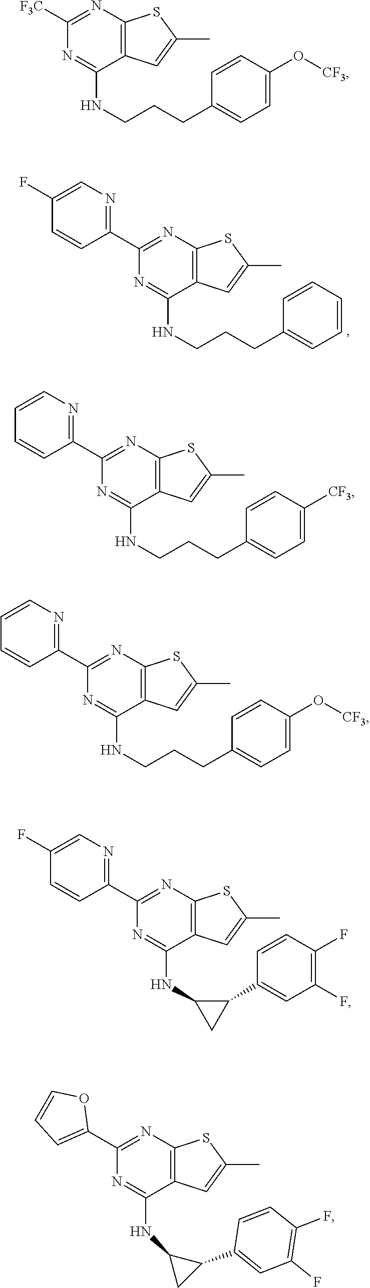 [SMILES: Cc1cc2c(NCCCc3ccc(OC(F)(F)F)cc3)nc(C(F)(F)F)nc2s1 ; Cc1cc2c(NCCCc3ccccc3)nc(-c3ccc(F)cn3)nc2s1 ; Cc1cc2c(NCCCc3ccc(C(F)(F)F)cc3)nc(-c3ccccn3)nc2s1 ; Cc1cc2c(NCCCc3ccc(OC(F)(F)F)cc3)nc(-c3ccccn3)nc2s1 ; Cc1cc2c(N[C@@H]3C[C@H]3c3ccc(F)c(F)c3)nc(-c3ccc(F)cn3)nc2s1 ; Cc1cc2c(N[C@@H]3C[C@H]3c3ccc(F)c(F)c3)nc(-c3ccco3)nc2s1]

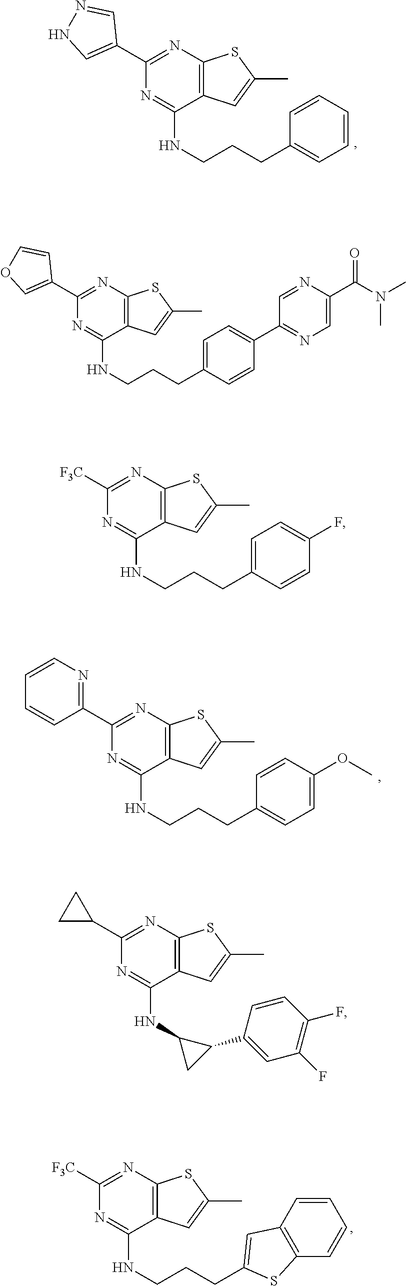 [SMILES: Cc1cc2c(NCCCc3ccccc3)nc(-c3cn[nH]c3)nc2s1 ; Cc1cc2c(NCCCc3ccc(-c4cnc(C(=O)N(C)C)cn4)cc3)nc(-c3ccoc3)nc2s1 ; Cc1cc2c(NCCCc3ccc(F)cc3)nc(C(F)(F)F)nc2s1 ; COc1ccc(CCCNc2nc(-c3ccccn3)nc3sc(C)cc23)cc1 ; Cc1cc2c(N[C@@H]3C[C@H]3c3ccc(F)c(F)c3)nc(C3CC3)nc2s1 ; Cc1cc2c(NCCCc3cc4ccccc4s3)nc(C(F)(F)F)nc2s1]

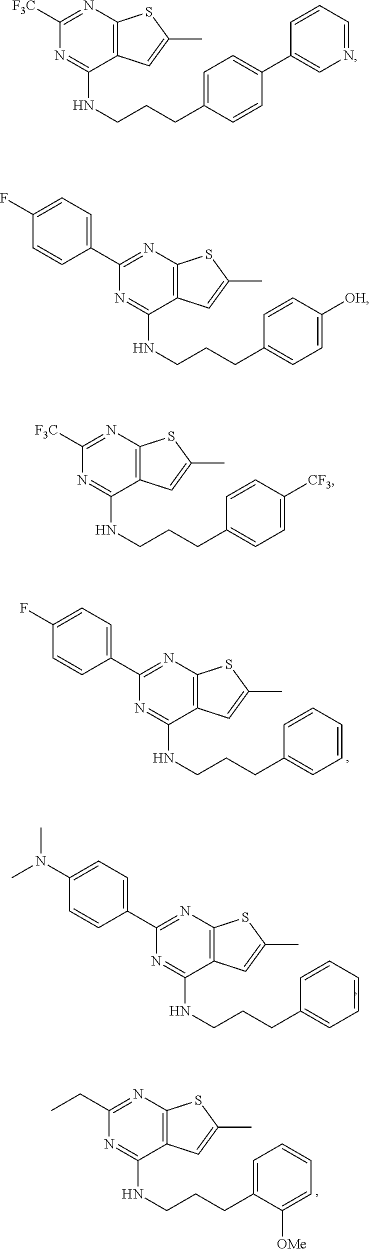 [SMILES: Cc1cc2c(NCCCc3ccc(-c4cccnc4)cc3)nc(C(F)(F)F)nc2s1 ; Cc1cc2c(NCCCc3ccc(O)cc3)nc(-c3ccc(F)cc3)nc2s1 ; Cc1cc2c(NCCCc3ccc(C(F)(F)F)cc3)nc(C(F)(F)F)nc2s1 ; Cc1cc2c(NCCCc3ccccc3)nc(-c3ccc(F)cc3)nc2s1 ; Cc1cc2c(NCCCc3ccccc3)nc(-c3ccc(N(C)C)cc3)nc2s1 ; CCc1nc(NCCCc2ccccc2OC)c2cc(C)sc2n1]

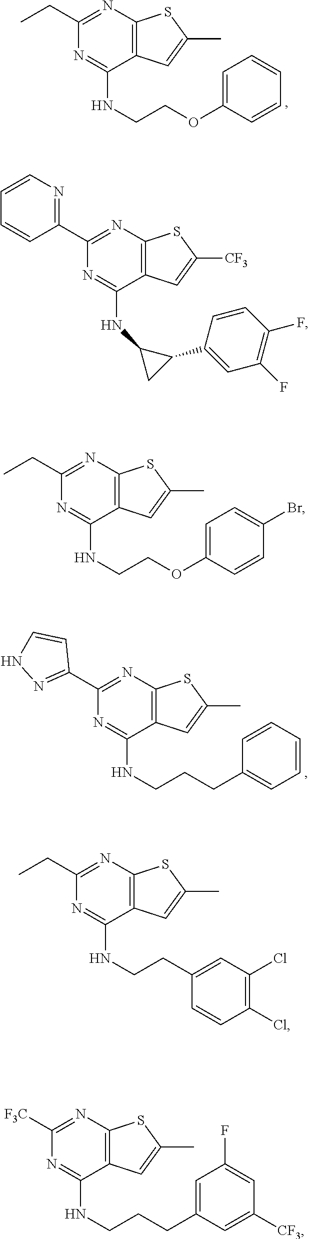 [SMILES: CCc1nc(NCCOc2ccccc2)c2cc(C)sc2n1 ; Fc1ccc([C@@H]2C[C@H]2Nc2nc(-c3ccccn3)nc3sc(C(F)(F)F)cc23)cc1F ; CCc1nc(NCCOc2ccc(Br)cc2)c2cc(C)sc2n1 ; Cc1cc2c(NCCCc3ccccc3)nc(-c3cc[nH]n3)nc2s1 ; CCc1nc(NCCc2ccc(Cl)c(Cl)c2)c2cc(C)sc2n1 ; Cc1cc2c(NCCCc3cc(F)cc(C(F)(F)F)c3)nc(C(F)(F)F)nc2s1]

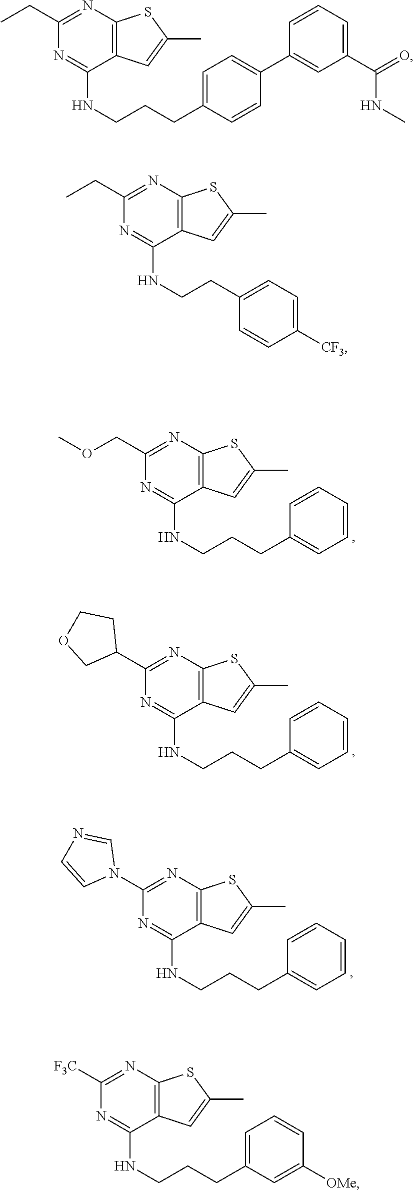

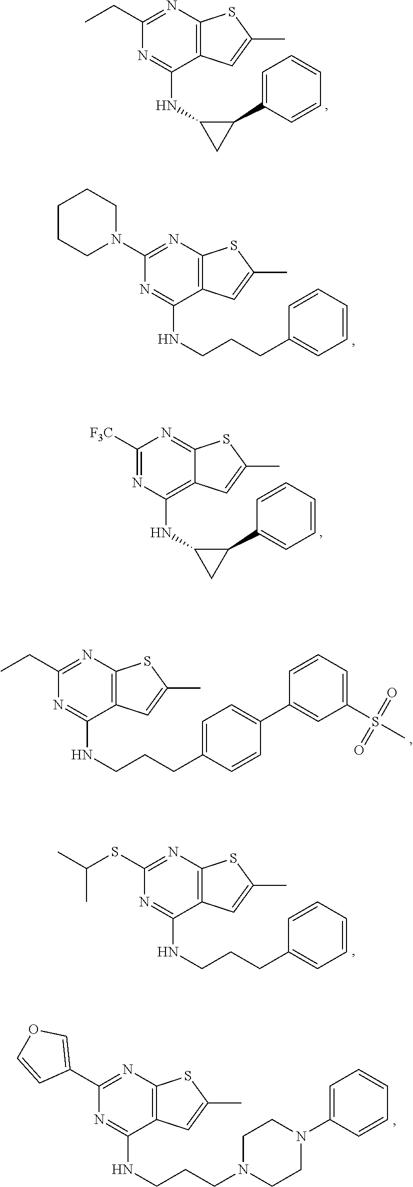

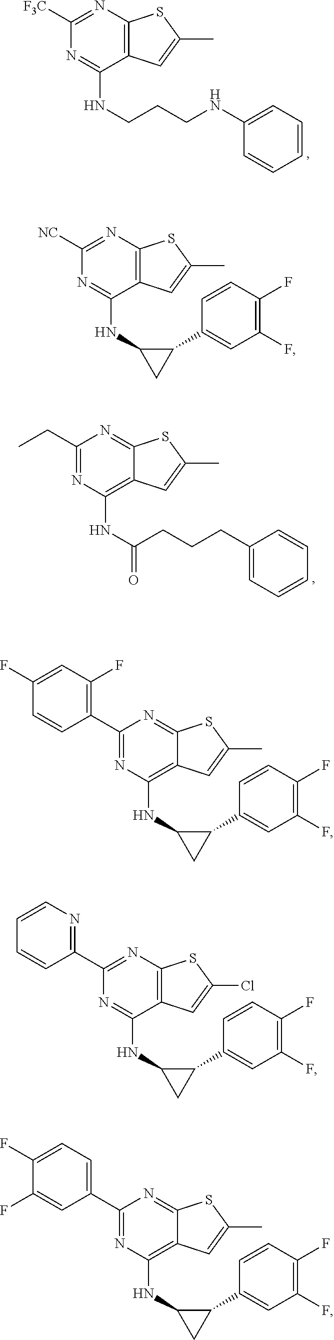 [SMILES: Cc1cc2c(NCCCNc3ccccc3)nc(C(F)(F)F)nc2s1 ; Cc1cc2c(N[C@@H]3C[C@H]3c3ccc(F)c(F)c3)nc(C#N)nc2s1 ; CCc1nc(NC(=O)CCCc2ccccc2)c2cc(C)sc2n1 ; Cc1cc2c(N[C@@H]3C[C@H]3c3ccc(F)c(F)c3)nc(-c3ccc(F)cc3F)nc2s1 ; Fc1ccc([C@@H]2C[C@H]2Nc2nc(-c3ccccn3)nc3sc(Cl)cc23)cc1F ; Cc1cc2c(N[C@@H]3C[C@H]3c3ccc(F)c(F)c3)nc(-c3ccc(F)c(F)c3)nc2s1]

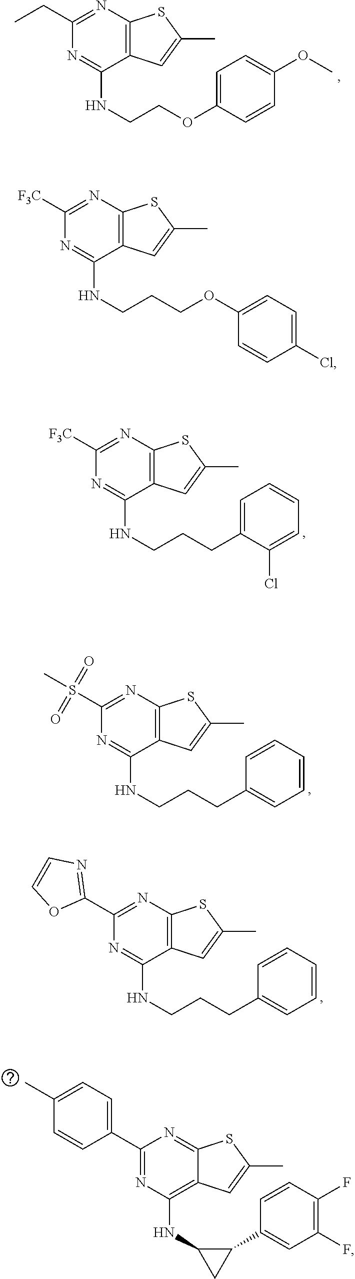

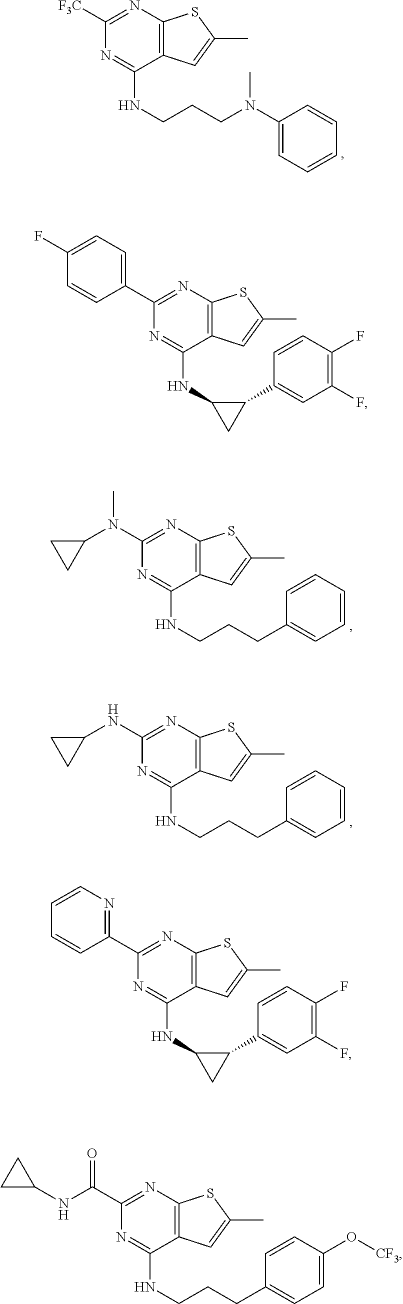 [SMILES: Cc1cc2c(NCCCN(C)c3ccccc3)nc(C(F)(F)F)nc2s1 ; Cc1cc2c(N[C@@H]3C[C@H]3c3ccc(F)c(F)c3)nc(-c3ccc(F)cc3)nc2s1 ; Cc1cc2c(NCCCc3ccccc3)nc(N(C)C3CC3)nc2s1 ; Cc1cc2c(NCCCc3ccccc3)nc(NC3CC3)nc2s1 ; Cc1cc2c(N[C@@H]3C[C@H]3c3ccc(F)c(F)c3)nc(-c3ccccn3)nc2s1 ; Cc1cc2c(NCCCc3ccc(OC(F)(F)F)cc3)nc(C(=O)NC3CC3)nc2s1]

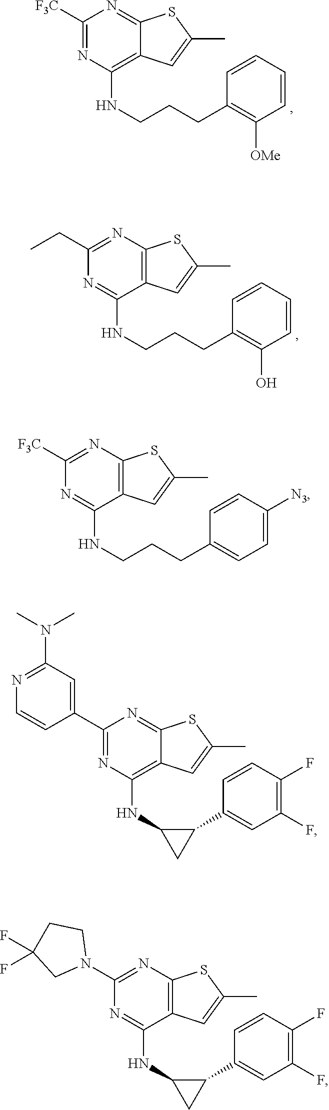 [SMILES: COc1ccccc1CCCNc1nc(C(F)(F)F)nc2sc(C)cc12 ; CCc1nc(NCCCc2ccccc2O)c2cc(C)sc2n1 ; Cc1cc2c(NCCCc3ccc(N=[N+]=[N-])cc3)nc(C(F)(F)F)nc2s1 ; Cc1cc2c(N[C@@H]3C[C@H]3c3ccc(F)c(F)c3)nc(-c3ccnc(N(C)C)c3)nc2s1 ; Cc1cc2c(N[C@@H]3C[C@H]3c3ccc(F)c(F)c3)nc(N3CCC(F)(F)C3)nc2s1]

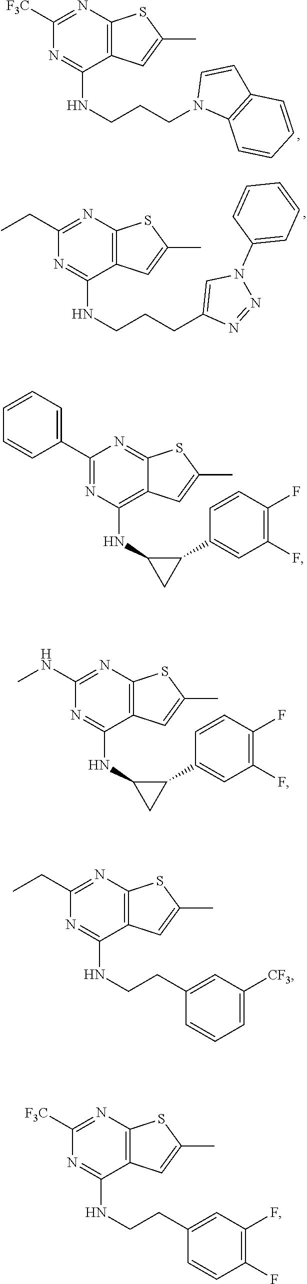

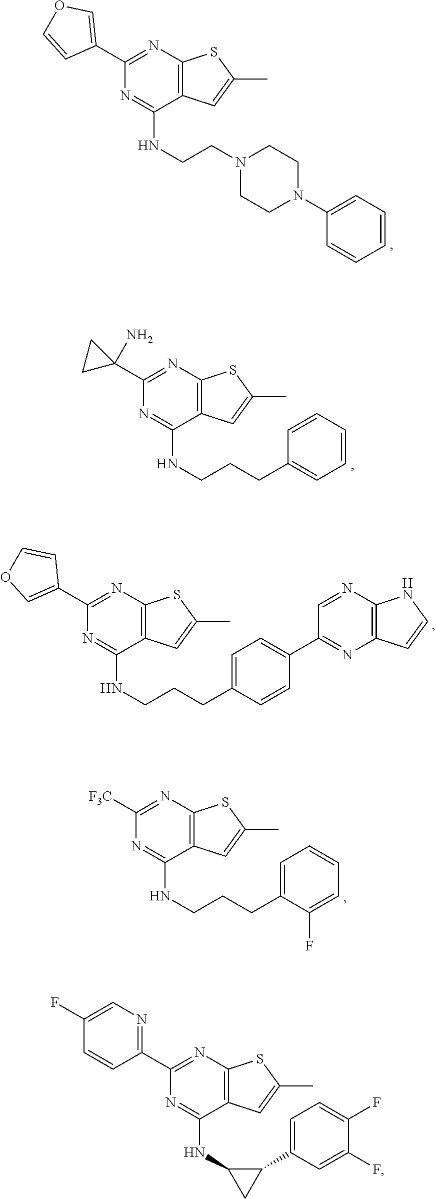 [SMILES: Cc1cc2c(NCCN3CCN(c4ccccc4)CC3)nc(-c3ccoc3)nc2s1 ; Cc1cc2c(NCCCc3ccccc3)nc(C3(N)CC3)nc2s1 ; Cc1cc2c(NCCCc3ccc(-c4cnc5[nH]ccc5n4)cc3)nc(-c3ccoc3)nc2s1 ; Cc1cc2c(NCCCc3ccccc3F)nc(C(F)(F)F)nc2s1 ; Cc1cc2c(N[C@@H]3C[C@H]3c3ccc(F)c(F)c3)nc(-c3ccc(F)cn3)nc2s1]

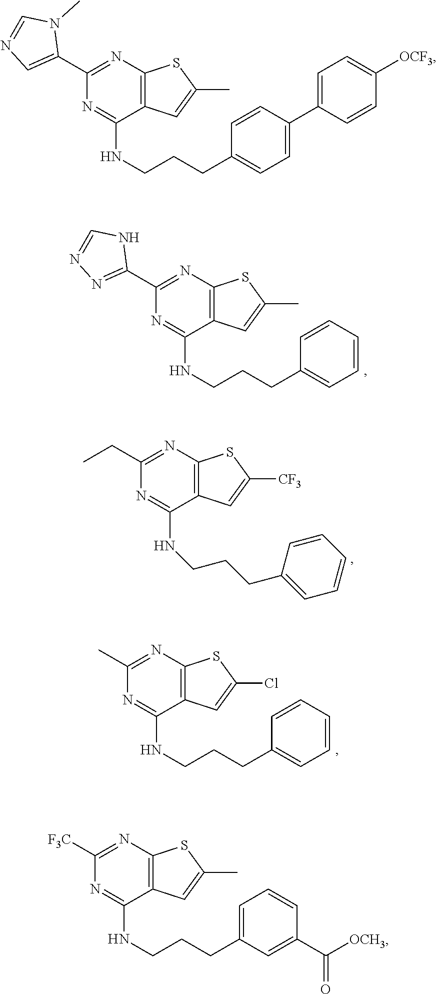 [SMILES: Cc1cc2c(NCCCc3ccc(-c4ccc(OC(F)(F)F)cc4)cc3)nc(-c3cncn3C)nc2s1 ; Cc1cc2c(NCCCc3ccccc3)nc(-c3nnc[nH]3)nc2s1 ; CCc1nc(NCCCc2ccccc2)c2cc(C(F)(F)F)sc2n1 ; Cc1nc(NCCCc2ccccc2)c2cc(Cl)sc2n1 ; COC(=O)c1cccc(CCCNc2nc(C(F)(F)F)nc3sc(C)cc23)c1]

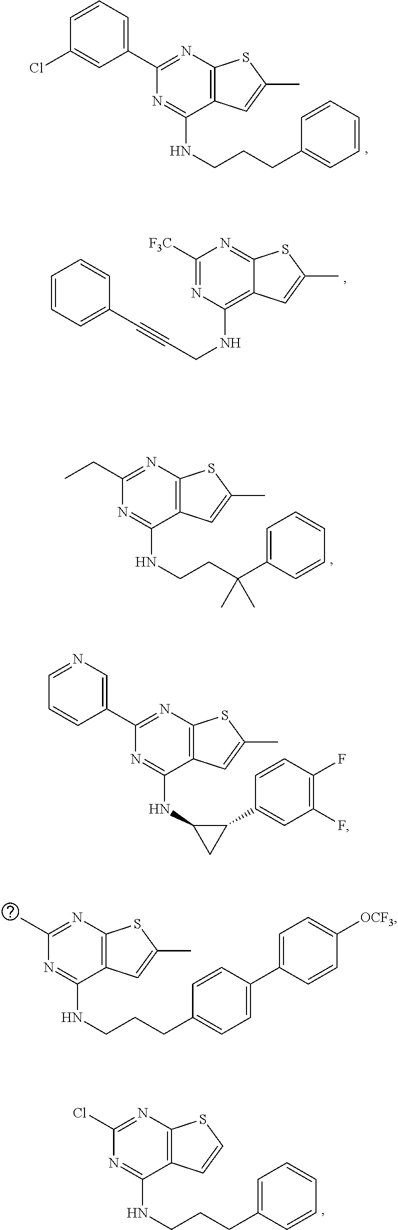

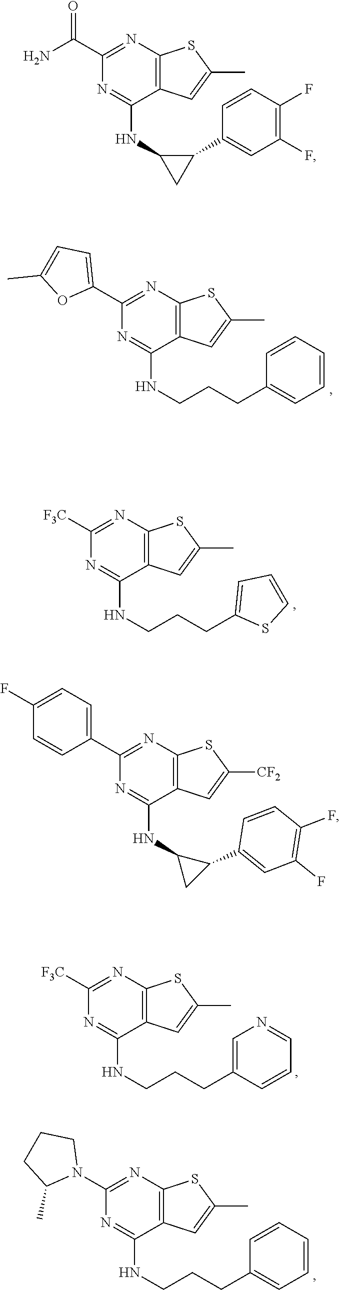

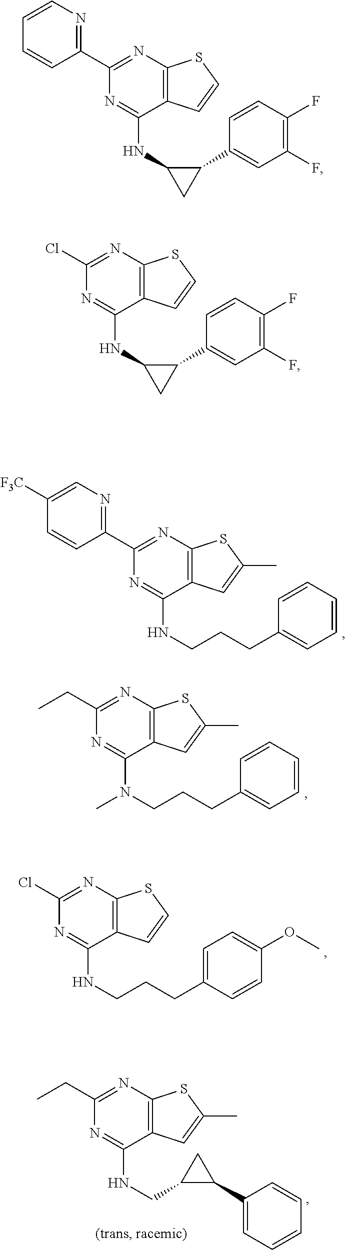

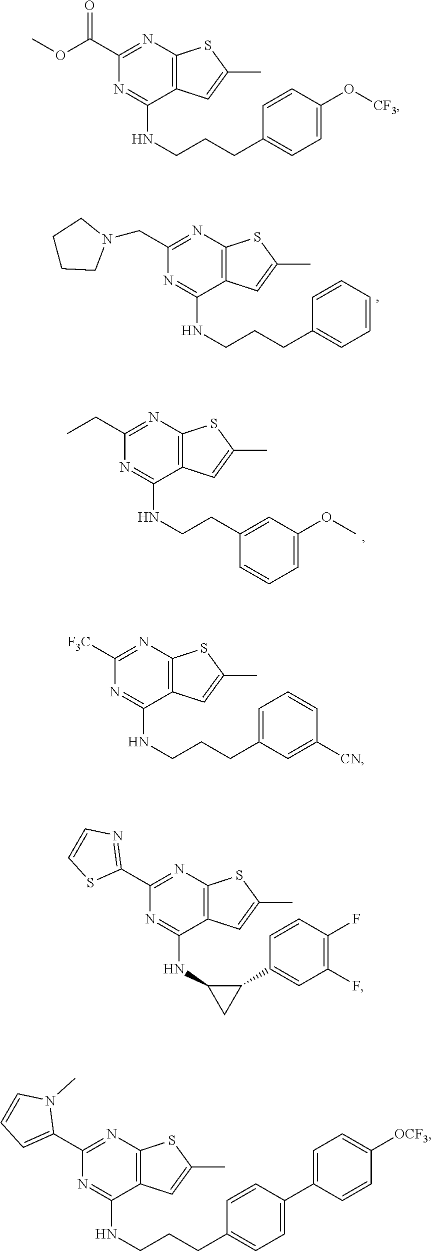 [SMILES: COC(=O)c1nc(NCCCc2ccc(OC(F)(F)F)cc2)c2cc(C)sc2n1 ; Cc1cc2c(NCCCc3ccccc3)nc(CN3CCCC3)nc2s1 ; CCc1nc(NCCc2cccc(OC)c2)c2cc(C)sc2n1 ; Cc1cc2c(NCCCc3cccc(C#N)c3)nc(C(F)(F)F)nc2s1 ; Cc1cc2c(N[C@@H]3C[C@H]3c3ccc(F)c(F)c3)nc(-c3nccs3)nc2s1 ; Cc1cc2c(NCCCc3ccc(-c4ccc(OC(F)(F)F)cc4)cc3)nc(-c3cccn3C)nc2s1]

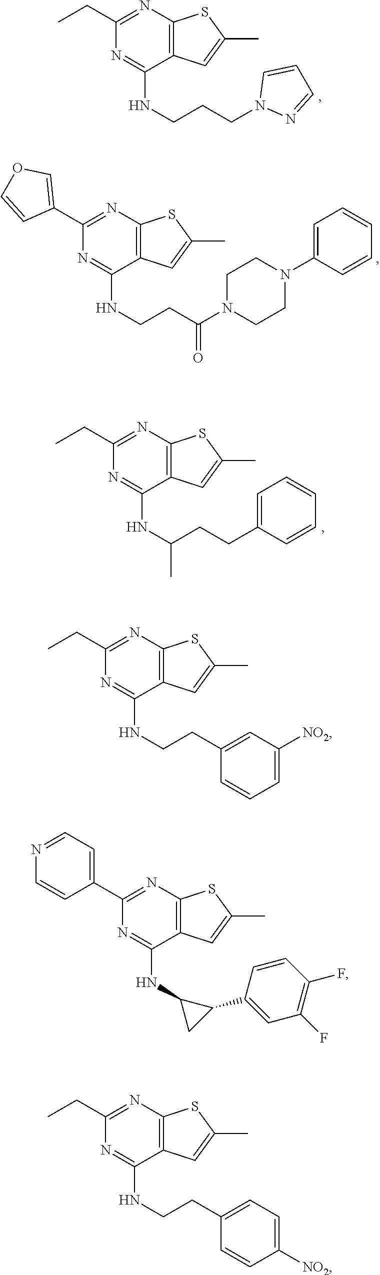 [SMILES: CCc1nc(NCCCn2cccn2)c2cc(C)sc2n1 ; Cc1cc2c(NCCC(=O)N3CCN(c4ccccc4)CC3)nc(-c3ccoc3)nc2s1 ; CCc1nc(NC(C)CCc2ccccc2)c2cc(C)sc2n1 ; CCc1nc(NCCc2cccc([N+](=O)[O-])c2)c2cc(C)sc2n1 ; Cc1cc2c(N[C@@H]3C[C@H]3c3ccc(F)c(F)c3)nc(-c3ccncc3)nc2s1 ; CCc1nc(NCCc2ccc([N+](=O)[O-])cc2)c2cc(C)sc2n1]

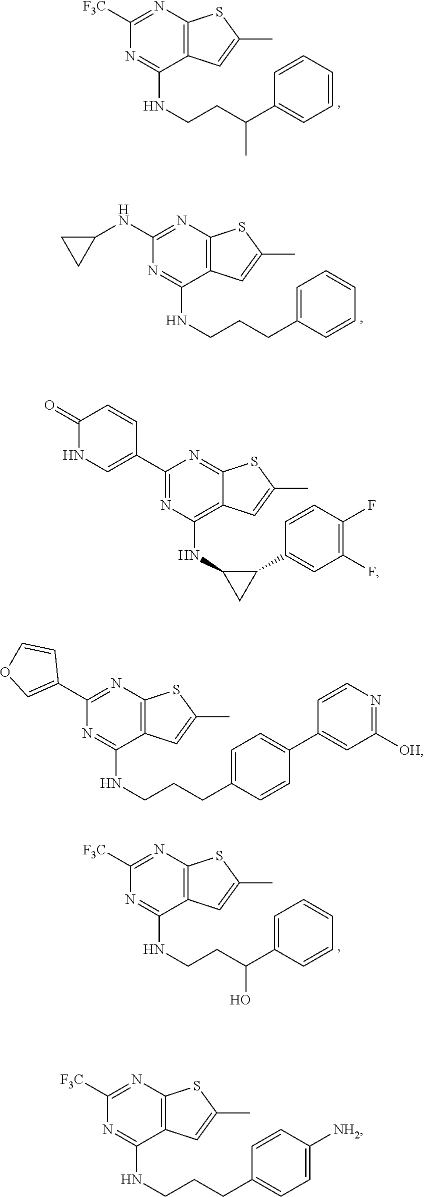 [SMILES: Cc1cc2c(NCCC(C)c3ccccc3)nc(C(F)(F)F)nc2s1 ; Cc1cc2c(NCCCc3ccccc3)nc(NC3CC3)nc2s1 ; Cc1cc2c(N[C@@H]3C[C@H]3c3ccc(F)c(F)c3)nc(-c3ccc(=O)[nH]c3)nc2s1 ; Cc1cc2c(NCCCc3ccc(-c4ccnc(O)c4)cc3)nc(-c3ccoc3)nc2s1 ; Cc1cc2c(NCCC(O)c3ccccc3)nc(C(F)(F)F)nc2s1 ; Cc1cc2c(NCCCc3ccc(N)cc3)nc(C(F)(F)F)nc2s1]

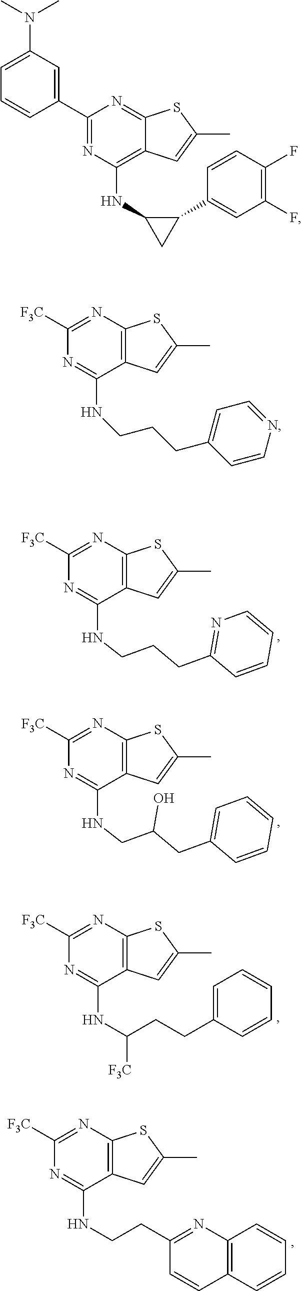 [SMILES: Cc1cc2c(N[C@@H]3C[C@H]3c3ccc(F)c(F)c3)nc(-c3cccc(N(C)C)c3)nc2s1 ; Cc1cc2c(NCCCc3ccncc3)nc(C(F)(F)F)nc2s1 ; Cc1cc2c(NCCCc3ccccn3)nc(C(F)(F)F)nc2s1 ; Cc1cc2c(NCC(O)Cc3ccccc3)nc(C(F)(F)F)nc2s1 ; Cc1cc2c(NC(CCc3ccccc3)C(F)(F)F)nc(C(F)(F)F)nc2s1 ; Cc1cc2c(NCCc3ccc4ccccc4n3)nc(C(F)(F)F)nc2s1]

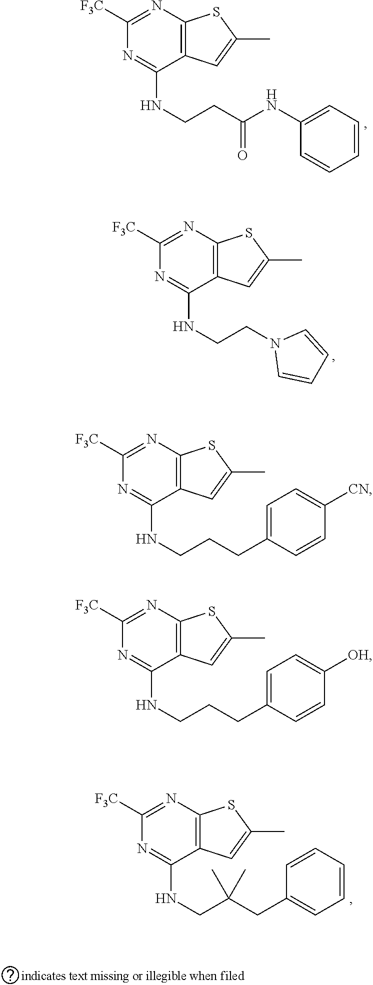 [SMILES: Cc1cc2c(NCCC(=O)Nc3ccccc3)nc(C(F)(F)F)nc2s1 ; Cc1cc2c(NCCn3cccc3)nc(C(F)(F)F)nc2s1 ; Cc1cc2c(NCCCc3ccc(C#N)cc3)nc(C(F)(F)F)nc2s1 ; Cc1cc2c(NCCCc3ccc(O)cc3)nc(C(F)(F)F)nc2s1 ; Cc1cc2c(NCC(C)(C)Cc3ccccc3)nc(C(F)(F)F)nc2s1]

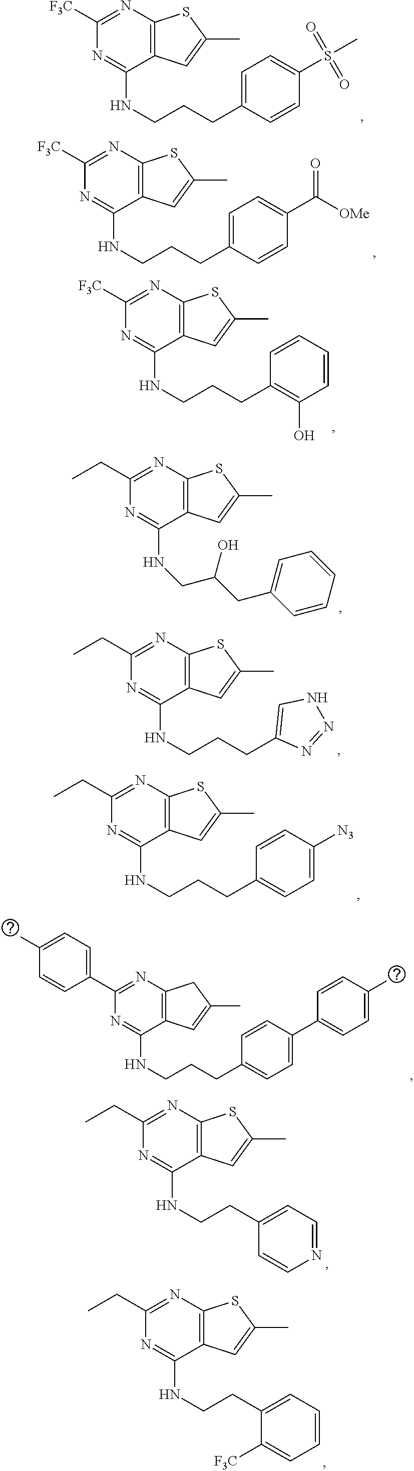

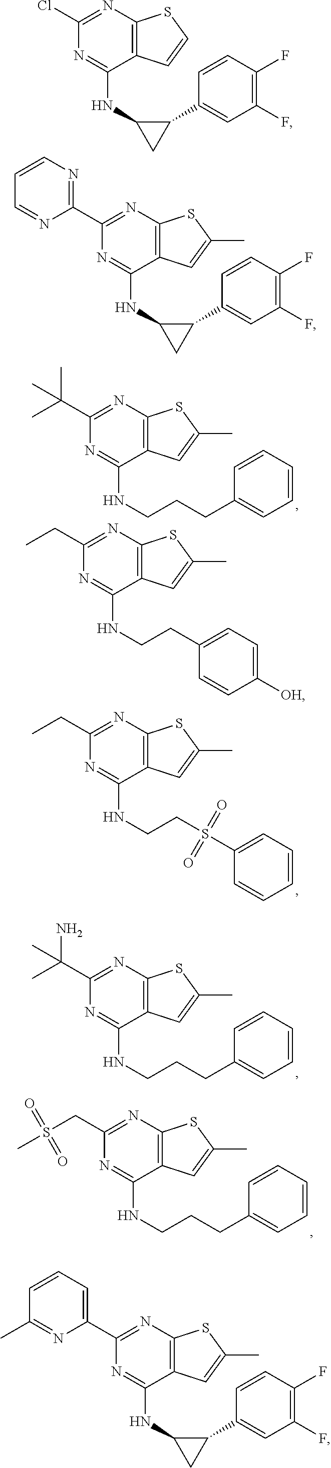

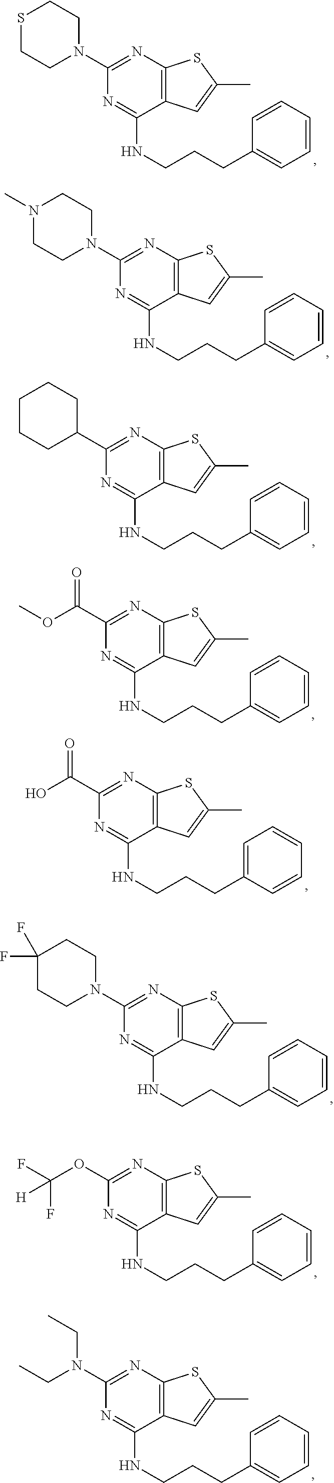

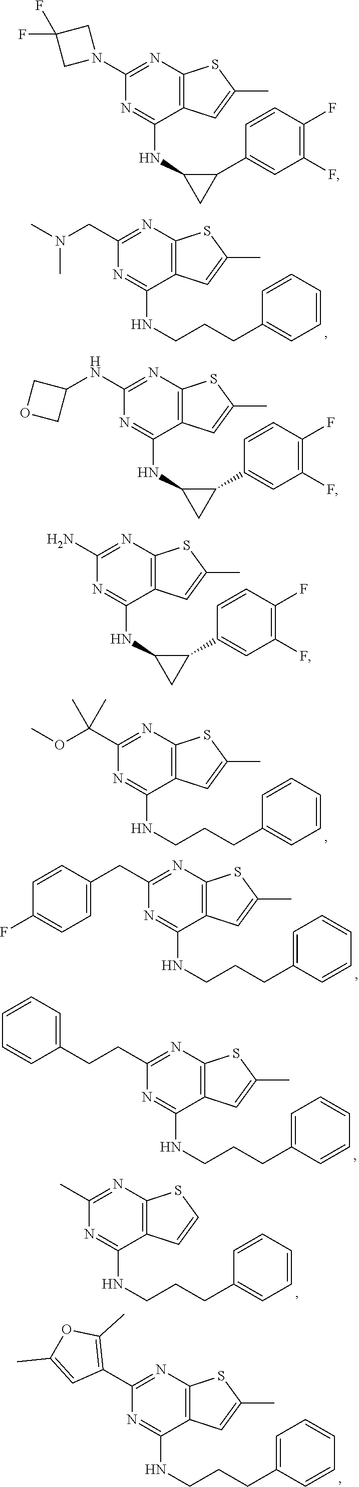

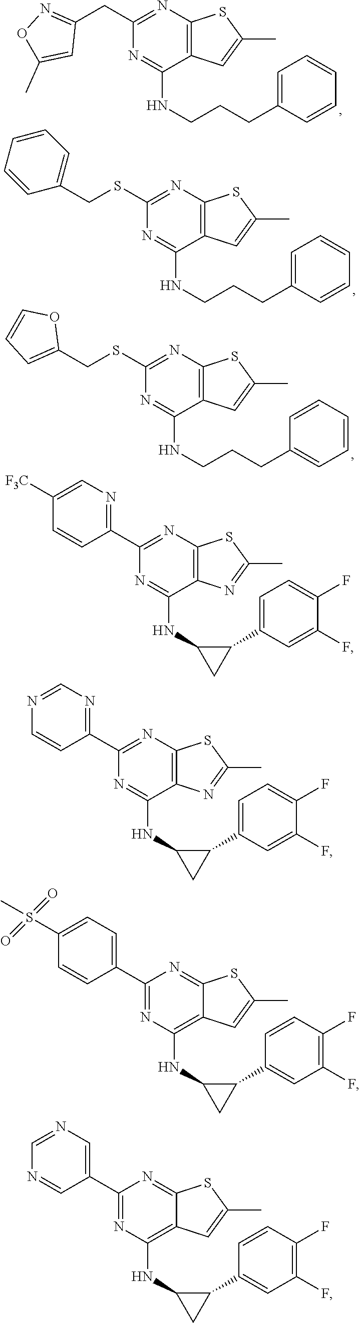

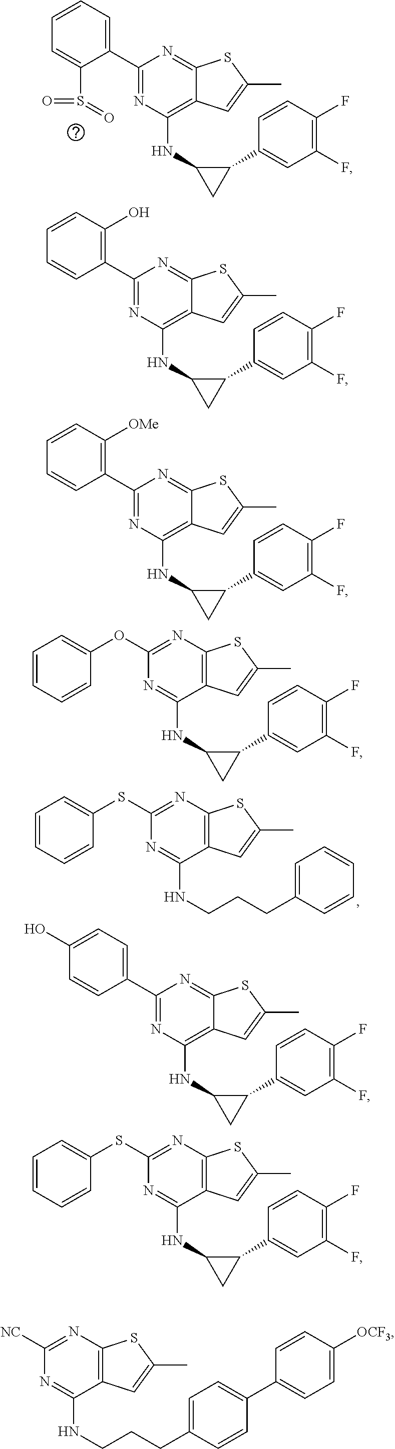

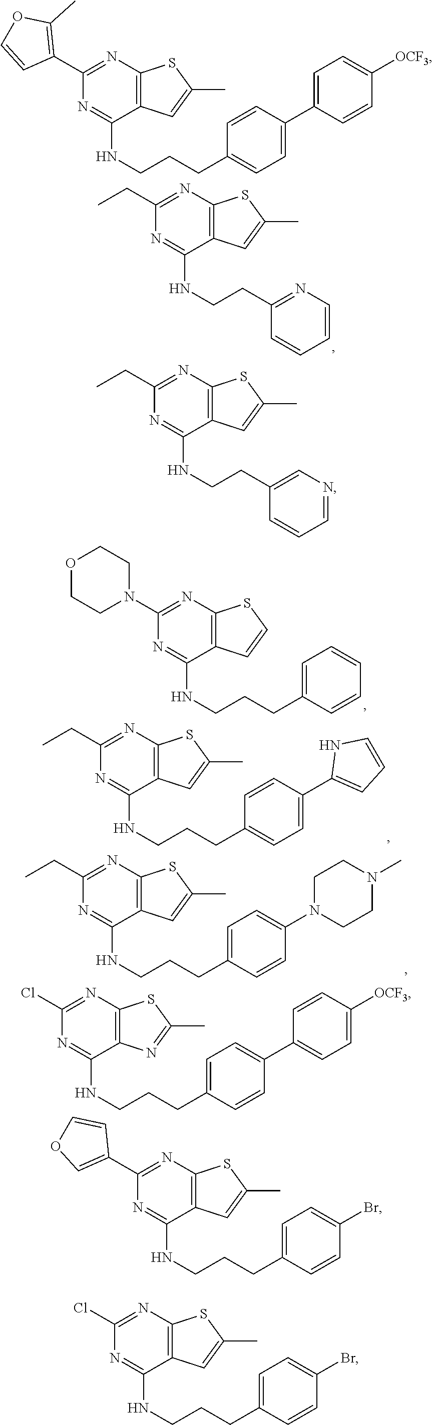

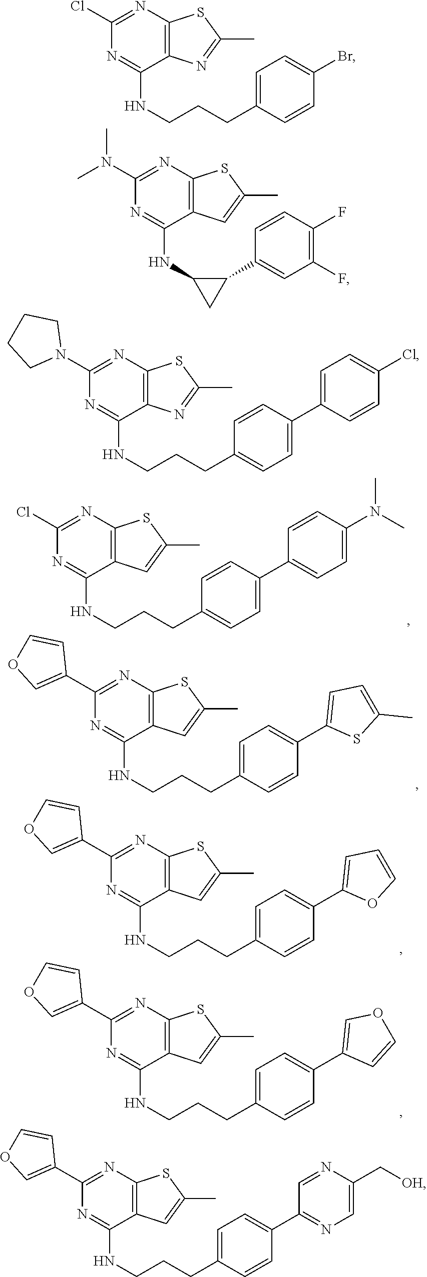

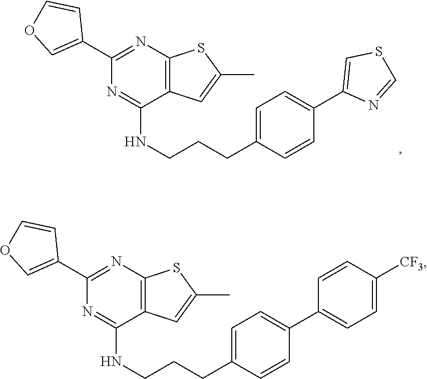 [SMILES: Cc1cc2c(NCCCc3ccc(-c4cscn4)cc3)nc(-c3ccoc3)nc2s1 ; Cc1cc2c(NCCCc3ccc(-c4ccc(C(F)(F)F)cc4)cc3)nc(-c3ccoc3)nc2s1]

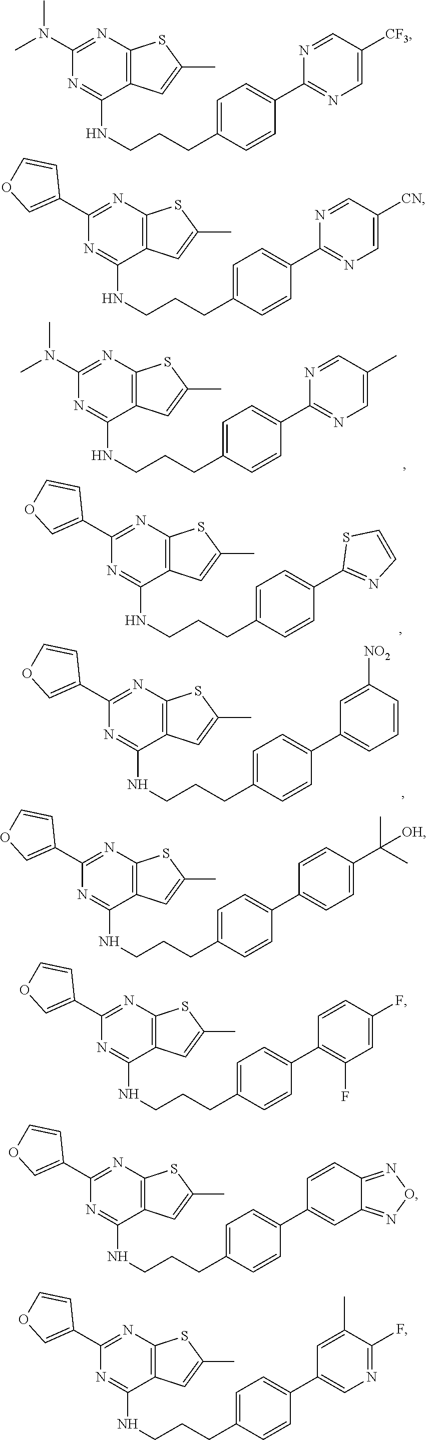

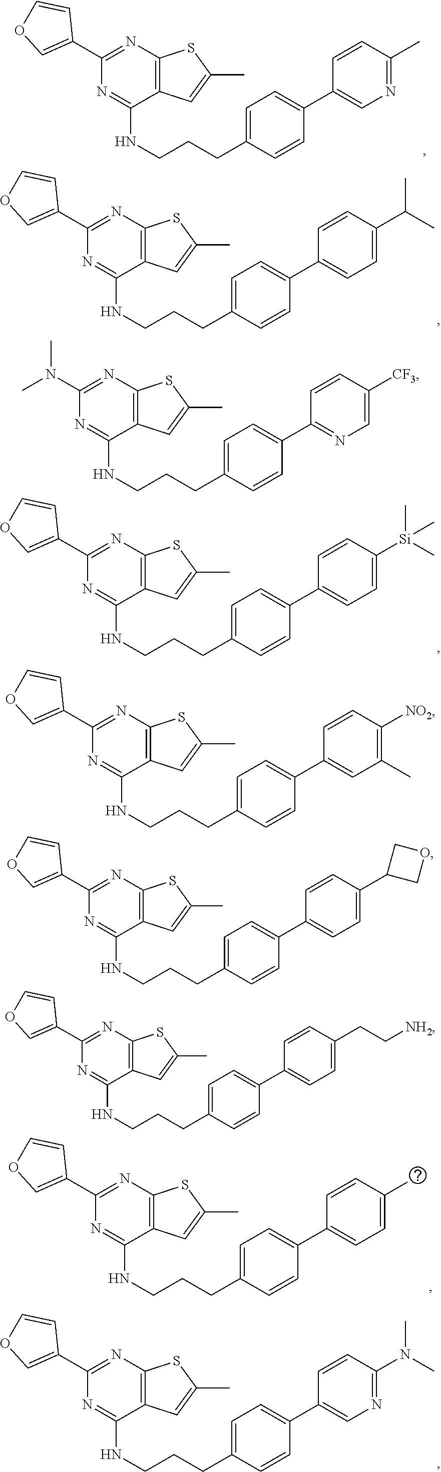

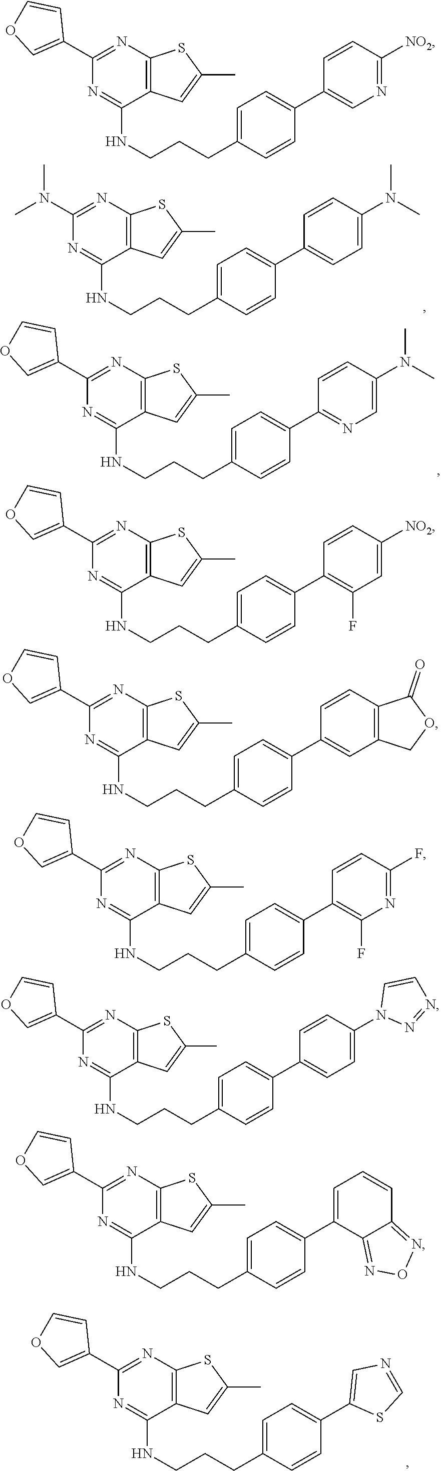

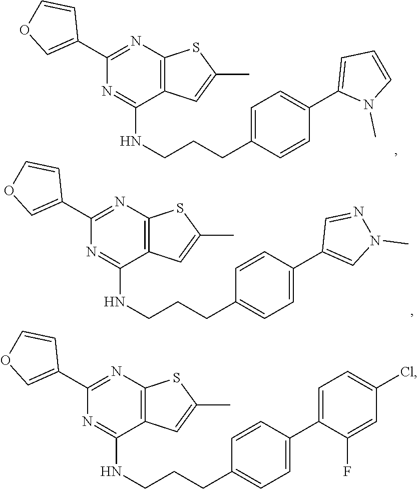 [SMILES: Cc1cc2c(NCCCc3ccc(-c4cccn4C)cc3)nc(-c3ccoc3)nc2s1 ; Cc1cc2c(NCCCc3ccc(-c4cnn(C)c4)cc3)nc(-c3ccoc3)nc2s1 ; Cc1cc2c(NCCCc3ccc(-c4ccc(Cl)cc4F)cc3)nc(-c3ccoc3)nc2s1]

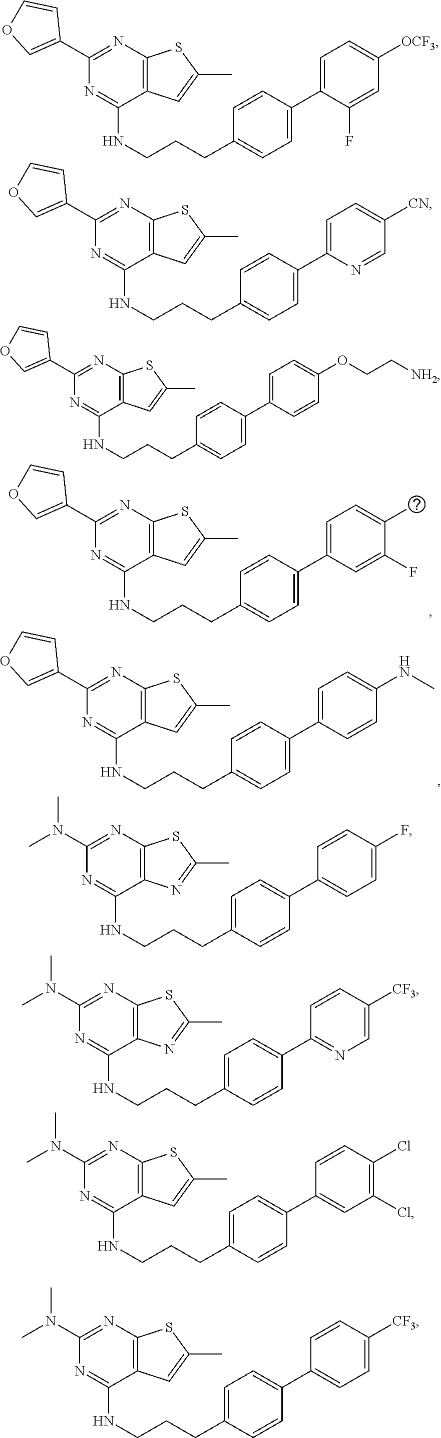

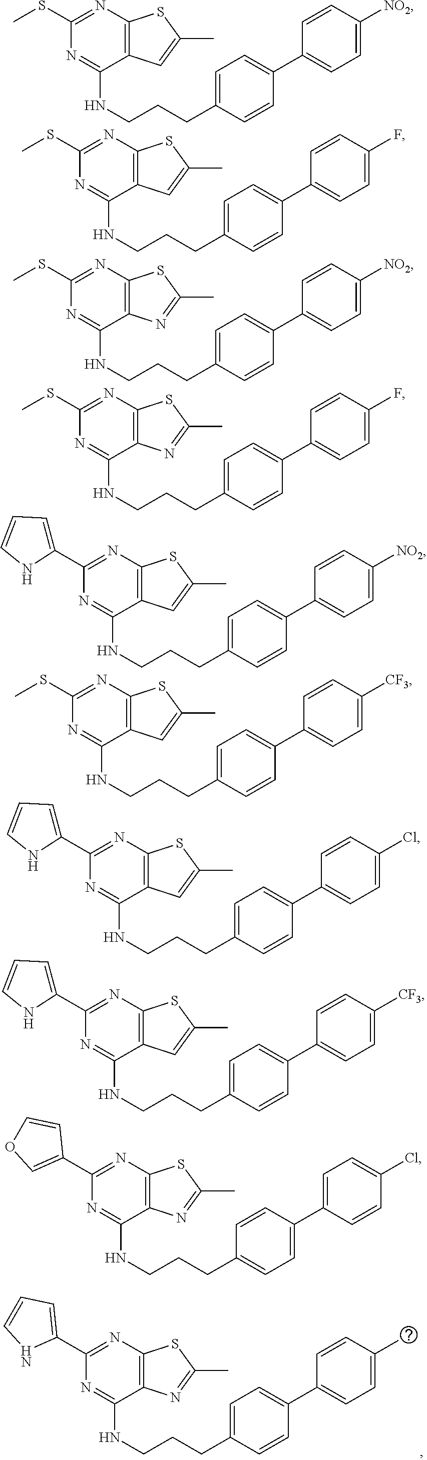

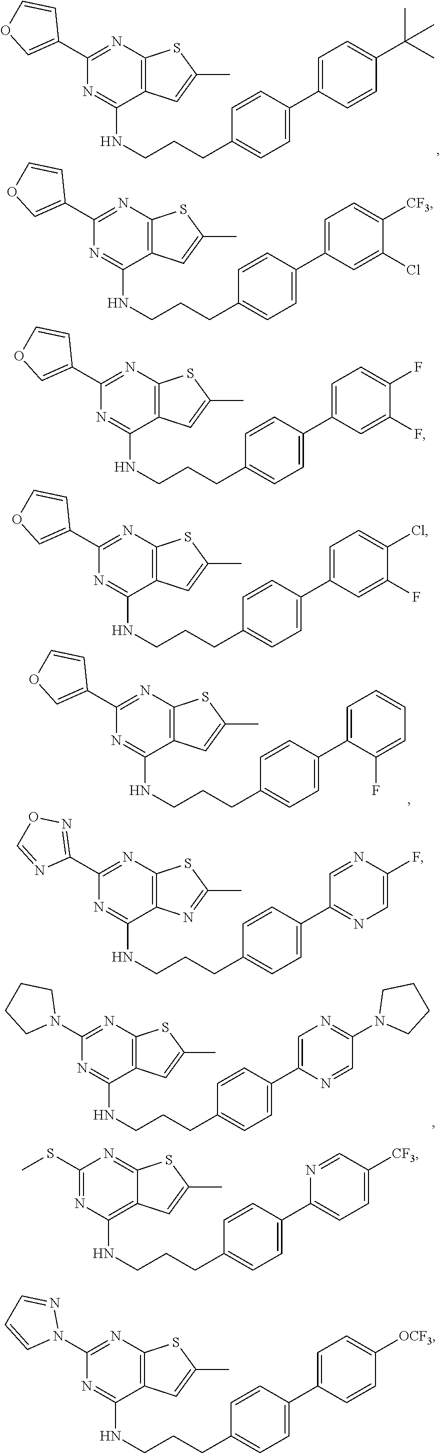

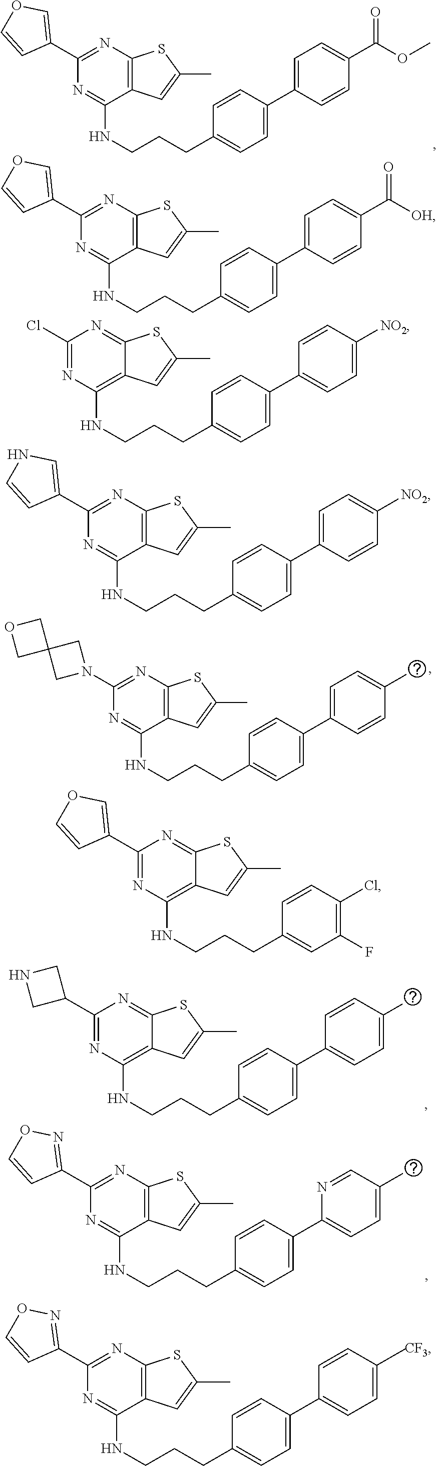

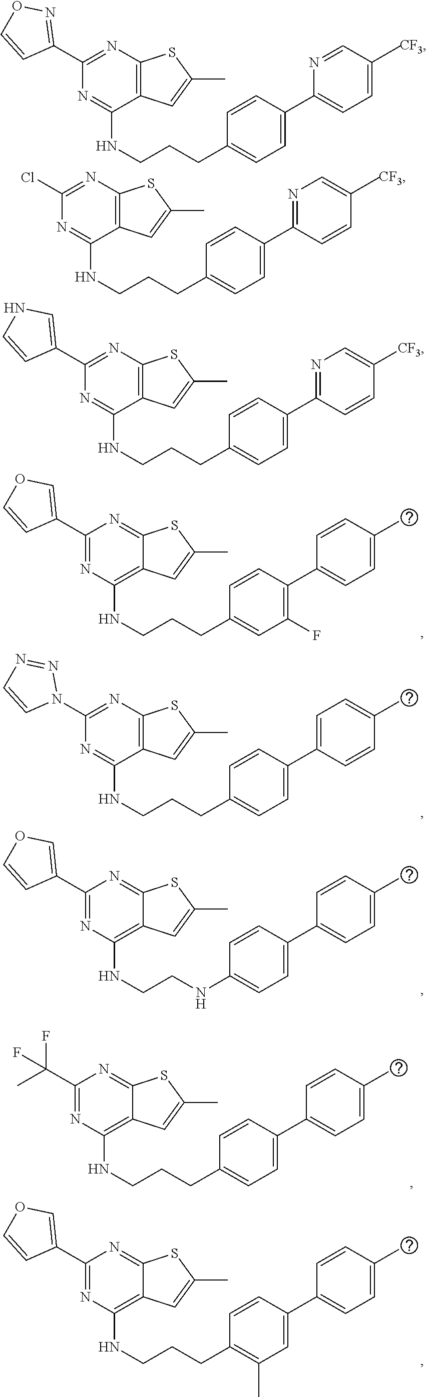

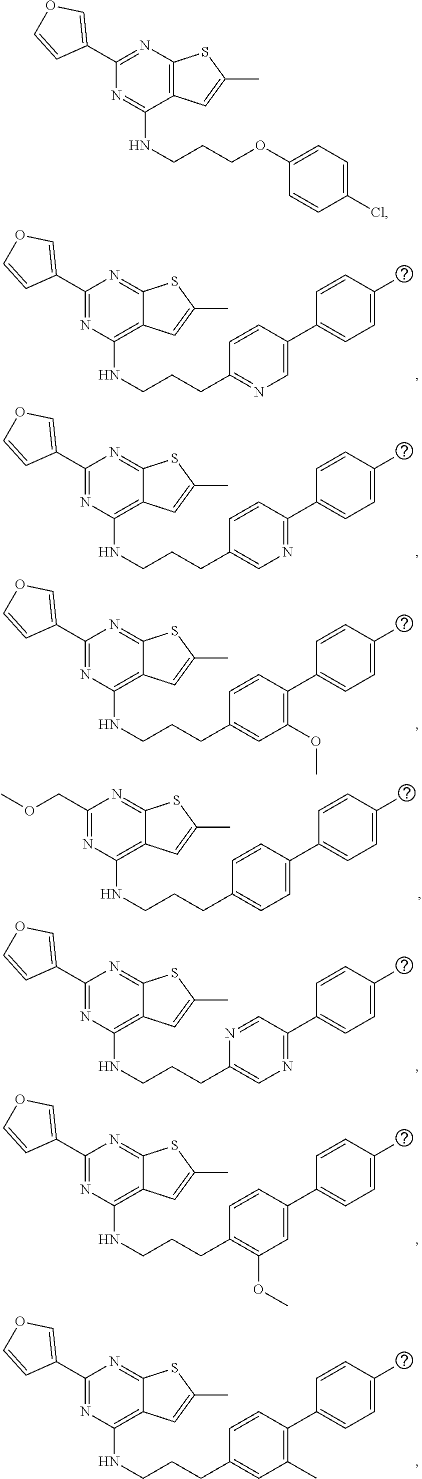

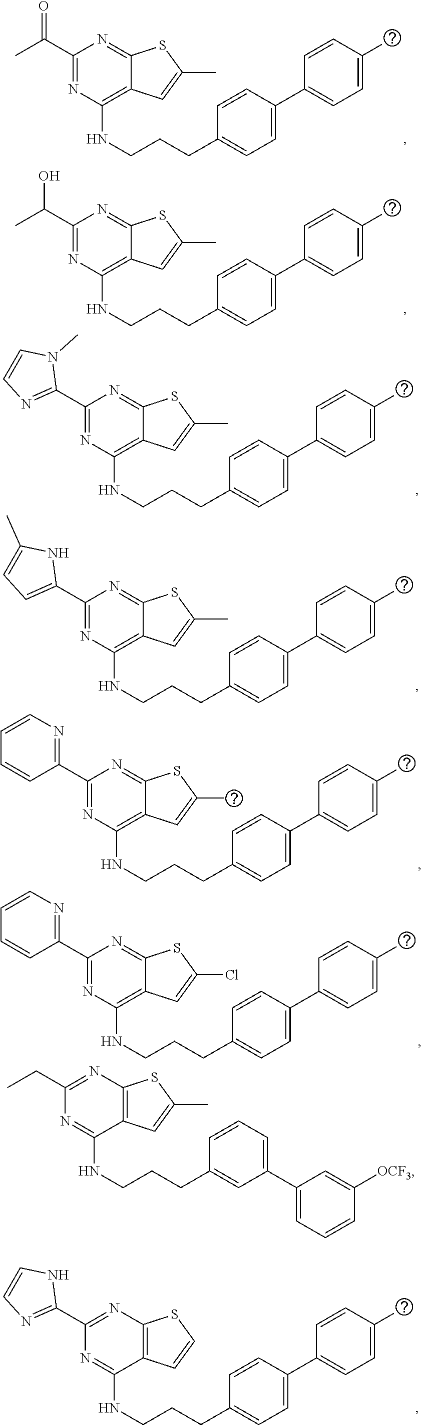

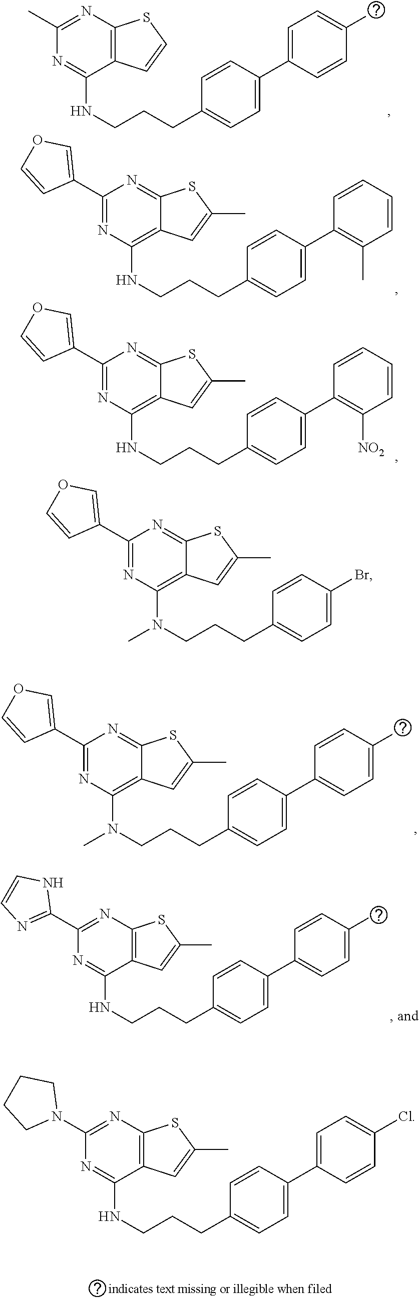

The compounds described herein can be made using the synthetic methods outlined in the Examples section. These methods can be further modified using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified for preparative, pilot- or large-scale production, either batch of continuous, using the principles and techniques of process chemistry as applied by a person skilled in the art. Such principles and techniques are taught, for example, in Anderson, Practical Process Research & Development A Guide for Organic Chemists (2012), which is incorporated by reference herein.

Compounds of the present disclosure can be useful for the prevention and treatment of one or more diseases or disorders discussed herein or otherwise. One or more of the compounds characterized or exemplified herein as an intermediate, a metabolite, and/or prodrug, may nevertheless also be useful for the prevention and treatment of one or more diseases or disorders. Suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

The compounds of the present disclosure can have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present disclosure may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. A single diastereomer can also be obtained. The chiral centers of the compounds of the present disclosure can have the S or the R configuration.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Compounds of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

The particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

Organic compounds can form complexes with solvents in which they are reacted or from which they are precipitated or crystallized. These complexes are known as “solvates.” Where the solvent is water, the complex is known as a “hydrate.” Many organic compounds can exist in more than one solid form, including crystalline and amorphous forms. Solid forms of the compounds provided herein, including any solvates thereof are within the scope of the present disclosure.

### Pharmaceutical Compositions and Methods of Use

Other aspects of the present disclosure are directed to a pharmaceutical composition comprising a compound as described herein. Further aspects of the present disclosure are directed to various methods of using the compounds and pharmaceutical compositions as described herein. For example, another aspect relates to a method of treating tuberculosis in a subject in need thereof comprising administering to the subject a therapeutically effective amount of a compound as described herein or a pharmaceutical composition as described herein. Other methods include those for inhibiting the replication of a Mycobacterium tuberculosis bacterium and/or inducing the death of a Mycobacterium tuberculosis bacterium comprising contacting the bacteria with an effective amount of a compound as described herein or a pharmaceutical composition as described herein.

The pharmaceutical composition can comprise an excipient. For example, the pharmaceutical composition can be formulated with one or more excipients for various routes of administration including: orally, intra-adiposally, intra-arterially, intra-articularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularly, intravitreally, liposomally, locally, mucosally, parenterally, rectally, subconjunctivally, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion.

For the purpose of administration to a patient in need of such treatment, pharmaceutical formulations (also referred to as pharmaceutical preparations, pharmaceutical compositions, pharmaceutical products, medicinal products, medicines, medications, or medicaments) comprise a therapeutically effective amount of a compound of the present disclosure typically formulated with one or more excipients and/or drug carriers appropriate to the indicated route of administration. The compounds of the present disclosure can be formulated in a manner amenable for the treatment of human and/or veterinary patients. Formulation can comprise admixing or combining one or more of the compounds of the present disclosure with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. The pharmaceutical formulation may be tableted or encapsulated, e.g., for oral administration. The compounds may be dissolved or slurried in water, polyethylene glycol, propylene glycol, ethanol, corn oil, cottonseed oil, peanut oil, sesame oil, benzyl alcohol, sodium chloride, and/or various buffers. Pharmaceutical formulations may be subjected to conventional pharmaceutical operations, such as sterilization and/or may contain drug carriers and/or excipients such as preservatives, stabilizers, wetting agents, emulsifiers, encapsulating agents such as lipids, dendrimers, polymers, proteins such as albumin, or nucleic acids, and buffers, etc.

Pharmaceutical formulations may be administered by a variety of methods, e.g., orally or by injection (e.g. subcutaneous, intravenous, intraperitoneal, etc.). Depending on the route of administration, the compounds of the present disclosure may be coated in a material to protect the compound from the action of acids and other natural conditions which may inactivate the compound. To administer the active compound by other than parenteral administration, it may be necessary to coat the compound with, or co-administer the compound with, a material to prevent its inactivation. For example, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds of the present disclosure may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds of the present disclosure can be administered orally, for example, with an inert diluent or an edible carrier. The compounds and other ingredients may also be enclosed in a hard or soft shell gelatin capsule, compressed into tablets, or incorporated directly into the subject's diet. For oral therapeutic administration, the compounds of the present disclosure may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, or mucosa. Alternatively, if local delivery to the lungs is desired the therapeutic compound may be administered by inhalation in a dry-powder or aerosol formulation.

It may be advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the disclosure can be dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. Active compounds can be administered at a therapeutically effective dosage sufficient to treat a condition associated with a condition in a patient. For example, the efficacy of a compound can be evaluated in an animal model system that may be predictive of efficacy in treating the disease in a human or another animal.

The effective dose range for the therapeutic compound can be extrapolated from effective doses determined in animal studies for a variety of different animals. In general, a human equivalent dose (HED) in mg/kg can be calculated in accordance with the following formula (see, e.g., Reagan-Shaw et al., FASEB J, 22(3):659-661, 2008, which is incorporated herein by reference): HED (mg/kg)=Animal dose (mg/kg)×(Animal Kₘ/Human Kₘ). Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

The therapeutically effective amount typically can vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. The amount can be less than 10,000 mg per day with a range of 750 mg to 9,000 mg per day.

The amount of the active compound in the pharmaceutical formulation can be from about 2 to about 75 weight percent. The amount can be from about 25 to about 60 weight percent.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. The agent can also be administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. The disclosure provides that the agent(s) may be taken orally and that the timing of which is or is not dependent upon food intake. Thus, for example, the agent can be taken every morning and/or every evening, regardless of when the subject has eaten or will eat.

In addition to being used as a monotherapy, the compounds of the present disclosure may also find use in combination therapies. Effective combination therapy may be achieved with a single composition or pharmacological formulation that includes more than one agent, or with two distinct compositions or formulations, administered at the same time, wherein one composition includes a compound of this disclosure, and the other includes one or more additional agent(s). Alternatively, the therapy may precede or follow the additional agent(s) treatment by intervals ranging from minutes to months.

An antibiotic may be administered in combination with the compounds of the present disclosure in order to treat a TB infection. In some cases, the TB infection may be a drug resistant strain which may be treated with a combination of multiple antibiotics. Some exemplary antibiotics and other therapeutic agents include isoniazid, pyrazinamide, rifampicin, ethambutol, levofloxacin, moxifloxacin, gatifloxacin, kanamycin, amikacin, capreomycin, streptomycin, ethionamide, prothionamide, cycloserine, terizidone, linezolid, clofazimine, bedaquiline, delamanid, para-aminosalicylic acid, imipenem, cilastatin, meropenem, or thiocetazone. The combination methods may comprise treating with one or more of rifampicin, pyrazinamide, ethambutol, and isoniazid. A therapy may comprise four of these antibiotics. Additionally, if resistance to one of these two antibiotics is detected, then bedaquiline or linezolid may also be administered instead of one or the above noted anitbiotics.

Finally, given the difficulty in treating TB infections, such combination therapies may be used for multiple months. Extremely resistant TB infections may be treated for 1 to 3 years in order to completely rid the body of the Mycobacterium tuberculosis bacterium completely. For less extensive or less difficult bacterial strains to treat, the treatments may also from 3 to 12 months instead of 1 to 3 years.

The tuberculosis can be caused by a multi-drug resistant mycobacteria and/or an extensively drug resistant mycobacteria.

A suitable subject is a mammal. A particularly suitable subject is a human.

A method of inhibiting QcrB, a subunit of the cytochrome bc₁-aa₃ supercomplex in Mtb in a subject in need thereof comprising administering to the subject a therapeutically effective amount of a compound of Formula I as disclosed herein.

The method can further comprise administering another anti-tuberculosis therapy. For example, additional anti-tuberculosis therapies can comprise at least one drug selected from the group consisting of ethambutol, isoniazid, pyrazinamide, rifampicin, streptomycin, an aminoglycoside, a polypeptide antibiotic, a fluoroquinolone, a thioamide, cycloserine, terizidone, rifabutin, a macrolide, linezolid, thioacetazone, thioridazine, arginine, vitamin D, bedaquiline, or a combination thereof.

A particularly suitable aminoglycoside comprises amikacin or kanamycin. Also for example, a particularly suitable polypeptide antibiotic comprises capreomycin, viomycin, or enviomycin. Also for example, a particularly suitable wherein the fluoroquinolone comprises ciprofloxacin, levofloxacin, or moxifloxacin. Also for example, a particularly suitable the thioamide comprises ethionamide or prothionamide. A particularly suitable macrolide comprises clarithromycin.

### Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “ [symbol] ” means a single bond, “ [symbol] ” means a double bond, and “ [symbol] ” means triple bond. The symbol “ [symbol] ” represents an optional bond, which if present is either single or double. The symbol “ [symbol] ” represents a single bond or a double bond. Thus, the formula

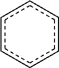

covers, for example,

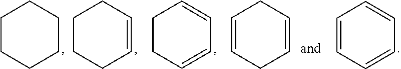

And, it is understood that no one such ring atom forms part of more than one double bond. Furthermore, the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

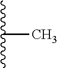

for methyl) indicates a point of attachment of the group. The point of attachment is typically only identified in this manner for larger groups in order to assist the reader in unambiguously identifying a point of attachment.

For the chemical groups and compound classes, the number of carbon atoms in the group or class is as indicated as follows: “Cn” defines the number (n) of carbon atoms in the group/class. “Cn-Cn′” defines both the minimum (n) and maximum number (n′) of carbon atoms in the group. Thus, C₂-C₁₀ alkyl designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₆ alkyl”, “C₅ alkyl” are synonymous. When any of the chemical groups or compound classes defined herein is modified by the term “substituted”, any carbon atom(s) in the moiety replacing a hydrogen atom is not counted. Thus methoxyphenyl, which has a total of seven carbon atoms, is an example of a substituted 6-membered aryl.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group refers to a planar unsaturated ring of atoms with 4n+2 electrons in a fully conjugated cyclic R system.

The term “alkyl” when used without the “substituted” modifier refers to a monovalent saturated aliphatic group with a carbon atom as the point of attachment, a linear or branched acyclic or cyclic structure (i.e., cycloalkyl), and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by, for example, —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂C₁, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C₁. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Additionally, the term “heteroalkyl” describes a group wherein one or more of the —CH₂— groups of the alkyl group is replaced by a heteroatom, particularly by O, S, NH, and the like.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

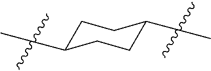 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by, for example, —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by, for example, —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by, for example, —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “-ene” as used as a suffix as part of another group denotes a bivalent radical in which a hydrogen atom is removed from each of two terminal carbons of the group. For example, alkylene denotes a bivalent alkyl group such as methylene (—CH₂—) or ethylene (—CH₂CH₂—). Alkenylene denotes a bivalent alkenyl group (i.e., having at least one double bond) such as propenylene (—CH═CH—). Alkynylene denotes a bivalent alkynyl group (i.e., having at least one triple bond) such as propynylene (—C≡C—). For clarity, addition of the -ene suffix is not intended to alter the definition of the principal word other than denoting a bivalent radical. Thus, continuing the example above, alkylene denotes an optionally substituted linear or branched bivalent hydrocarbon radical.

The term “aryl” when used without the “substituted” modifier refers to a monovalent unsaturated aromatic group with an aromatic carbon atom as the point of attachment, said carbon atom forming part of a one or more aromatic ring structure, wherein the ring atoms are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

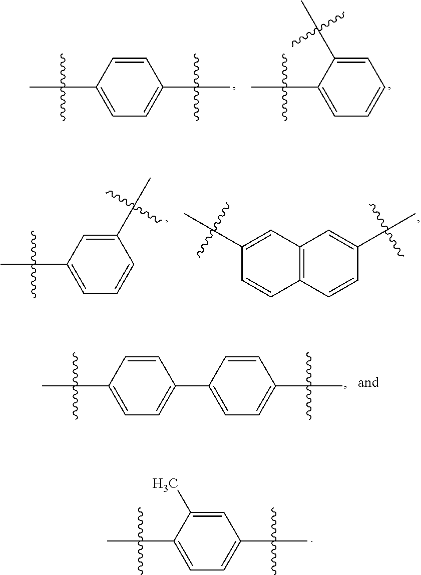

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by, for example, —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by, for example, —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. Unfused rings are connected with a covalent bond. As used herein, the term heteroaryl does not preclude the presence of one or more alkyl or aryl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl.

Pyridine and quinoline are non-limiting examples of heteroarenes. When these terms are used with the “substituted” modifier one or more hydrogen atom has been independently replaced by, for example, —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heteroaralkyl” when used without the “substituted” modifier refers to the monovalent group -alkanediyl-heteroaryl, in which the terms alkanediyl and aryl are each used in a manner consistent with the definitions provided above. When the term heteroaralkyl is used with the “substituted” modifier one or more hydrogen atom from the alkanediyl and/or the aryl group has been independently replaced by, for example, —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by, for example, —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy).

The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by, for example, —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. When any of these terms is used with the “substituted” modifier one or more hydrogen atom attached to a carbon atom has been independently replaced by, for example, —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

An “active ingredient” (AI) (also referred to as an active compound, active substance, active agent, pharmaceutical agent, agent, biologically active molecule, or a therapeutic compound) is the ingredient in a pharmaceutical drug or a pesticide that is biologically active. The similar terms active pharmaceutical ingredient (API) and bulk active are also used in medicine, and the term active substance may be used for pesticide formulations.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. The patient or subject can be a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical agent, pharmaceutical preparation, pharmaceutical composition, pharmaceutical formulation, pharmaceutical product, medicinal product, medicine, medication, medicament, or simply a drug) is a drug used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients (defined above) in pharmaceutical contexts.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present disclosure. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-b-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains <15%, more preferably <10%, even more preferably <5%, or most preferably <1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

Having described the invention in detail, it will be apparent that modifications and variations are possible without departing from the scope of the invention defined in the appended claims.

### EXAMPLES

The following non-limiting examples are provided to further illustrate the present disclosure.

### Example 1: General Procedures

### General Synthesis, Purification, and Analytical Chemistry Procedures

Starting materials, reagents and solvents were purchased from a vendor unless otherwise noted. All reactions utilized conventional heating, unless otherwise stated. ¹H NMR spectra were measured on a Varian 400 MHz NMR instrument. The following abbreviations were used to express the multiplicities: s=singlet; d=doublet; t=triplet; q=quartet; quin=quintet; m=multiplet; br=broad. High-performance liquid chromatography (HPLC) was carried out on a Teledyne ACCQ Prep HP125 using a Teledyne Redisep Prep C18 5 M, 20×150 mm reverse phase column, eluted with a gradient system of 5:95 to 95:5 acetonitrile:water with a buffer consisting of 0.05% formic acid. Following prep HPLC, compounds were passed through a Silicycle® Siliaprep carbonate column to neutralize any formic acid compound salts. Liquid chromatography mass spectra (LCMS) was performed on an Agilent 1100 series, model 1946d, HPLC/MSD, using electrospray ionization (ESI) for detection. Silica gel column chromatography was carried out on a Teledyne ISCO CombiFlash purification system using pre-packaged silica gel columns (4 g-330 g sizes), using either EtOAc:hexanes or MeOH:DCM gradient elution. All compounds used for biological assays are greater than 95% purity based on NMR and HPLC by absorbance at 210 nM and 254 nM wavelengths.

### Abbreviations

EtOAc=ethyl acetate; MeOH=methanol; EtOH=ethanol; iPrOH=isopropanol; DCM=dichloromethane; THF=tetrahydrofuran; DMF=dimethylformamide; DMSO=dimethylsulfoxide; DMAP=N,N-dimethylaminopyridine; DI=deionized; DIPEA=N,N-Diisopropylethylamine; mCPBA=meta-chloroperoxybenzoic acid.

### General Suzuki Protocol:

A flask with a stir bar, was heated to 250° C. under vacuum for 2 minutes, and then allowed to cool to room temperature under vacuum for an additional 10 minutes, after which time an N₂ atmosphere was continuously maintained. As described in specific examples, either Cs₂CO₃ or K₂CO₃ (2.5 eqv.) was added, followed by DI water (0.1 mL) and the reaction was stirred until the solids were dissolved. Dioxane (0.5 mL) was next added, followed by the desired aryl halide (or aryl boronate) (0.30 mmol) starting material, the appropriate aryl boronate (or aryl halide if using aryl boronate starting material) (2 eqv.) and Pd(PPh₃)₄ (0.15 eqv.). The reaction flask was evacuated and purged with N₂ (3×), then stirred for the time and temperature specified. Upon completion, the reaction was cooled to room temperature and the solvents were evaporated under reduced pressure. The crude residue was purified by prep HPLC or silica gel column chromatography, or a combination of the two, to give the pure final product.

### General Phosphorous Oxychloride (POCl₃) Protocol

To a threaded pressure vessel, was added the desired pyrimidinone starting material (1.0 mmol), followed by the slow addition of POCl₃ (2.0 mL). The vial was sealed and heated for the time and temperature described in specific examples. Upon completion, the reaction was cooled to room temperature, then carefully added to a vigorously stirring beaker of ice water. As the POCl₃ quenches, the ice will begin to melt, more ice can be used to ensure the aqueous solution does not warm over room temperature. 30% NH₄OH(aq) was then carefully added to neutralize the reaction mixture, again using ice to maintain ambient temperatures. Typically, the neutralized product precipitates and can easily be filtered, rinsing well with DI water. Occasionally an extraction into DCM (3×) is necessary to obtain the product, after which the organic layers are concentrated in vacuo and dried overnight under high vacuum. If the product is <95% pure (as determined by LCMS), further purification is carried out by silica gel column chromatography, otherwise chlorinated building blocks can be used as is.

### General Click Protocol

The appropriate azide (or alkyne) starting material (0.43 mmol) and the desired alkyne reagent (or azide for alkyne starting material) (6 eqv.) were dissolved in a mixture of THF (1 mL) and DMF (0.5 mL). Next, CuSO₄-5H₂O (0.2 eqv.) and (+) sodium L-ascorbate (0.4 eqv.) were each separately dissolved in DI water (0.25 mL) before sequentially adding each aqueous solution to the reaction. The reaction flask was then sealed and purged with N₂ and heated in until completion. Specific reaction conditions and product purification protocols are described in the examples.

### Example 2: Building Block Syntheses

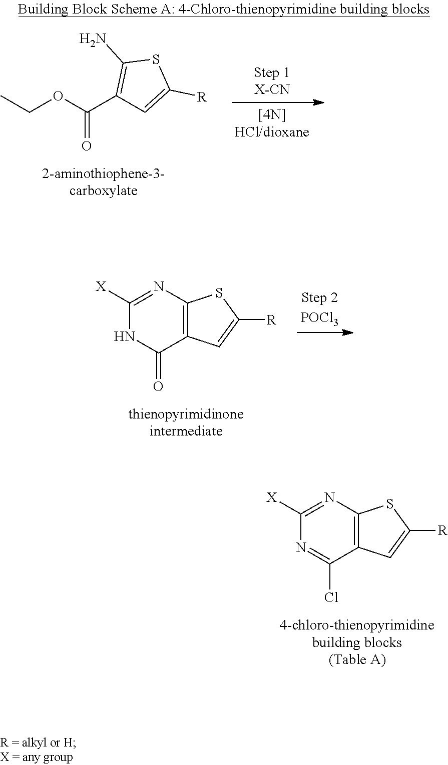

General Procedure Scheme A: Reaction conditions and compound data for building blocks examples are listed in Table A. Starting materials and reagents were commercially purchased.

Step 1: To a threaded pressure vessel, was added the appropriate 2-aminothiophene-3-carboxylate derivative (1.08 mmol), and X—CN (1.1 eqv), followed by the addition of anhydrous [4N] HCl/dioxane (3 mL). The vial was sealed and heated for the specified time and temperature (conditions in Table A). Upon completion, the reaction mixture was cooled to room temperature and a precipitate formed. The precipitate was filtered, rinsing with dioxane (2 mL) to obtain the product as a solid. If the reaction mixture was cooled to room temperature and no precipitate formed, saturated NaHCO₃ (aq) was added to neutralize the HCl, and the thienopyrimidinone intermediate was extracted into DCM (3×) and concentrated in vacuo. The intermediate was then dried overnight under high vacuum and used crude in step 2.

Step 2: Following the General POCl₃ Protocol, the thienopyrimidinone intermediate (from step 1), was reacted with POCl₃ for the specified time and temperature (conditions in Table A) to give the desired 4-chloro-thienopyrimidine building block (Table A).

TABLE A
Building blocks from Scheme A

| Int. | | Step 1 | Step 2 | Yield | | |
|---|---|---|---|---|---|---|
| No. | Structure | Conditions | Conditions | (2 steps) | IUPAC | Compound Data |
| KH 4007 | 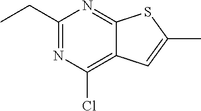 | 1 d (70° C.), 3 d (95° C.), 1 d (100° C.) | 1 d (100° C.) | 71% | 4-chloro-2-ethyl-6- methylthieno[2,3- d]pyrimidine | LCMS: ESI [M + H]⁺ = 213.1 |
| KH 4018 | 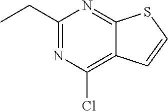 | 3 h (25° C.), 1 d (50° C.), 3 d (80° C.), 1 d (110° C.) | 2 h (100° C.) | 9% | 4-chloro-2-ethylthieno[2,3- d]pyrimidine | LCMS: ESI [M + H]⁺ = 199.0 |
| KH 4027 | 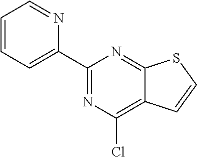 [SMILES: Clc1nc(-c2ccccn2)nc2sccc12] | 2 h (50° C.), 1 d (80° C.), 4 h (110° C.) | 2 h (100° C.) | 16% | 4-chloro-2-(pyridin-2- yl)thieno[2,3-d]pyrimidine | LCMS: ESI [M + H]⁺ = 248.0 |
| KH 4036 | 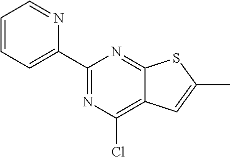 [SMILES: Cc1cc2c(Cl)nc(-c3ccccn3)nc2s1] | 1 d (70° C.), 1 h (90° C.) | 2 h (100° C.) | 80% | 4-chloro-6-methyl-2-(pyridin- 2-yl)thieno[2,3-d]pyrimidine | LCMS: ESI [M + H]⁺ = 262.0 |
| KH 4037 | 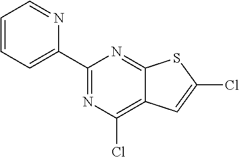 [SMILES: Clc1cc2c(Cl)nc(-c3ccccn3)nc2s1] | 1 d (70° C.), 1 h (90° C.) | 2 h (100° C.) | 71% | 4,6-dichloro-2-(pyridin-2- yl)thieno[2,3-d]pyrimidine | LCMS: ESI [M + H]⁺ = 282.0 |
| KH 4057 | 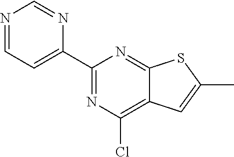 [SMILES: Cc1cc2c(Cl)nc(-c3ccncn3)nc2s1] | 1 d (70° C.), 1 h (100° C.) | 2 h (100° C.) | 54% | 4-chloro-6-methyl-2- (pyrimidin-4-yl)thieno[2,3- d]pyrimidine | LCMS: ESI [M + H]⁺ = 263.0 |
| KH 4069 | 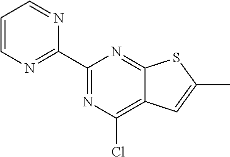 [SMILES: Cc1cc2c(Cl)nc(-c3ncccn3)nc2s1] | 1 d (70° C.), 2 h (100° C.) | 2 h (95° C.) | 47% | 4-chloro-6-methyl-2- (pyrimidin-2-yl)thieno[2,3- d]pyrimidine | LCMS: ESI [M + H]⁺ = 263.0 |
| KH 4070 | 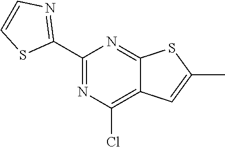 [SMILES: Cc1cc2c(Cl)nc(-c3nccs3)nc2s1] | 1 d (70° C.), 2 h (100° C.) | 2 h (95° C.) | 19% | 4-chloro-6-methyl-2-(thiazol- 2-yl)thieno[2,3-d]pyrimidine | LCMS: ESI [M + H]⁺ = 268.0 |
| KH 4076 | 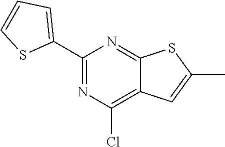 [SMILES: Cc1cc2c(Cl)nc(-c3cccs3)nc2s1] | 1 d (70° C.), 2 h (100° C.) | 2 h (100° C.) | 55% | 4-chloro-6-methyl-2- (thiophen-2-yl)thieno[2,3- d]pyrimidine | LCMS: ESI [M + H]⁺ = 267.0 |
| KH 4082 | 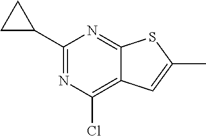 | 1 d (70° C.), 2 h (100° C.) | 2 h (95° C.) | 45% | 4-chloro-2-cyclopropyl-6- methylthieno[2,3- d]pyrimidine | LCMS: ESI [M + H]⁺ = 225.0 |
| KH 4083 | 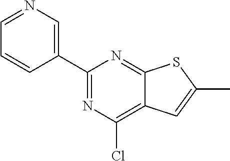 [SMILES: Cc1cc2c(Cl)nc(-c3cccnc3)nc2s1] | 1 d (70° C.), 2 h (100° C.) | 2 h (95° C.) | 67% | 4-chloro-6-methyl-2-(pyridin- 3-yl)thieno[2,3-d]pyrimidine | LCMS: ESI [M + H]⁺ = 262.0 |
| KH 4084 | 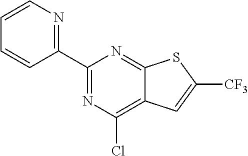 [SMILES: FC(F)(F)c1cc2c(Cl)nc(-c3ccccn3)nc2s1] | 1 d (70° C.), 2 h (100° C.) | 2 h (95° C.) | 36% | 4-chloro-2-(pyridin-2-yl)-6- (trifluoromethyl)thieno[2,3- d]pyrimidine | LCMS: ESI [M + H]⁺ = 316.0 |
| KH 4085 | 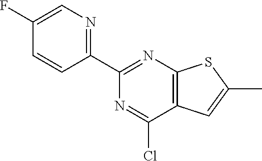 [SMILES: Cc1cc2c(Cl)nc(-c3ccc(F)cn3)nc2s1] | 1 d (70° C.) | 2 h (95° C.) | 27% | 4-chloro-2-(5-fluoropyridin- 2-yl)-6-methylthieno[2,3- d]pyrimidine | LCMS: ESI [M + H]⁺ = 280.0 |
| KH 4086 | 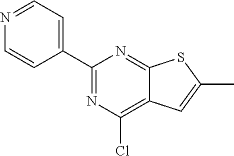 [SMILES: Cc1cc2c(Cl)nc(-c3ccncc3)nc2s1] | l d (70° C.), 2 d (100° C.) | 2 h (95° C.) | 34% | 4-chloro-6-methyl-2-(pyridin- 4-yl)thieno[2,3-d]pyrimidine | LCMS: ESI [M + H]⁺ = 262.0 |
| KH 4099 | 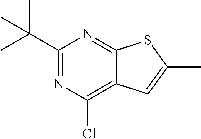 | 3 d (70° C.), 1 d (100° C.) | 2 h (95° C.) | 34% | 2-(tert-butyl)-4-chloro-6- methylthieno[2,3- d]pyrimidine | LCMS: ESI [M + H]⁺ = 241.1 |
| KH 4100 | 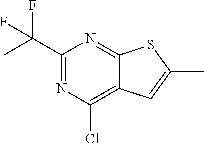 | 1 d (70° C.), 2 h (100° C.) | 2 h (95° C.) | 46% | 4-chloro-2-(1,1- difluoroethyl)-6- methylthieno[2,3- d]pyrimidine | LCMS: ESI [M + H]⁺ = 249.0 |
| KH 5001 | 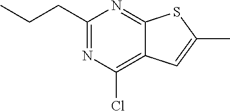 | 1 d (70° C.), 2 h (100° C.) | 2 h (95° C.) | 26% | 4-chloro-6-methyl-2- propylthieno[2,3- d]pyrimidine | LCMS: ESI [M + H]⁺ = 227.1 |
| KH 5002 | 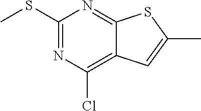 | 1 d (70° C.), 2 h (100° C.) | 2 h (95° C.) | 71% | 4-chloro-6-methyl-2- (methylthio)thieno[2,3- d]pyrimidine | LCMS: ESI [M + H]⁺ = 231.0 |
| KH 5015 | 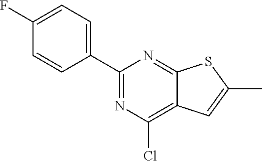 [SMILES: Cc1cc2c(Cl)nc(-c3ccc(F)cc3)nc2s1] | 1 d (70° C.), 1 d (90° C.) | 1 h (90° C.) | 89% | 4-chloro-2-(4-fluorophenyl)- 6-methylthieno[2,3- d]pyrimidine | LCMS: ESI [M + H]⁺ = 279.0 |
| KH 5016 | 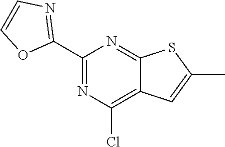 [SMILES: Cc1cc2c(Cl)nc(-c3ncco3)nc2s1] | 1 d (70° C.) | 2 h (90° C.) | 39% | 2-(4-chloro-6- methylthieno[2,3- d]pyrimidin-2-yl)oxazole | LCMS: ESI [M + H]⁺ = 252 |
| KH 5017 | 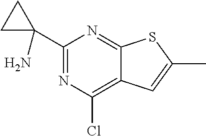 | 1 d (70° C.), 1 d (100° C.) | 2 h (90° C.) | 47% | 1-(4-chloro-6- methylthieno[2,3- d]pyrimidin-2- yl)cyclopropan-1-amine | LCMS: ESI [M + H]⁺ = 240.1 |
| KH 5044 | 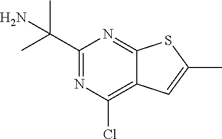 | 1 d (70° C.), 1 d (100° C.) | 2 h (90° C.) | 48% | 2-(4-chloro-6- methylthieno[2,3- d]pyrimidin-2-yl)propan-2- amine | LCMS: ESI [M + H]⁺ = 242.1 |
| KH 5045 | 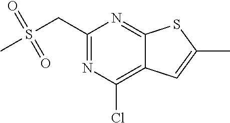 | 1 d (70° C.) | 2 h (90° C.) | 69% | 4-chloro-6-methyl-2- ((methylsulfonyl)methyl) thieno[2,3-d]pyrimidine | LCMS: ESI [M + H]⁺ = 277.0 |
| KH 5046 | 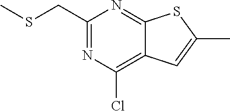 | 1 d (70° C.) 4 h (100° C.) | 2 h (90° C.) | 72% | 4-chloro-6-methyl-2- ((methylthio)methyl)thieno[2, 3-d]pyrimidine | LCMS: ESI [M + H]⁺ = 245.1 |
| KH 5047 | 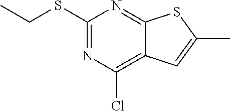 | 1 d (70° C.) | 2 h (90° C.) | 25% | 4-chloro-2-(ethylthio)-6- methylthieno[2,3- d]pyrimidine | LCMS: ESI [M + H]⁺ = 245.1 |
| KH 5048 | 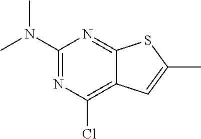 | 1 d (70° C.), 6 h (100° C.) | 2 h (90° C.) | 53% | 4-chloro-N,N,6- trimethylthieno[2,3- d]pyrimidin-2-amine | LCMS: ESI [M + H]⁺ = 228.1 |
| KH 5062 | 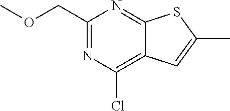 | 1 d (70° C.), 5 d (100° C.) | 2 h (90° C.) | 23% | 4-chloro-2-(methoxymethyl)- 6-methylthieno[2,3- d]pyrimidine | LCMS: ESI [M + H]⁺ = 229.1 |
| KH 5088 | 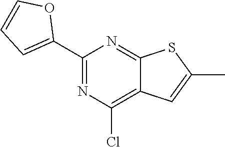 [SMILES: Cc1cc2c(Cl)nc(-c3ccco3)nc2s1] | 3 d (70° C.) | 1 h (90° C.) | 72% | 4-chloro-2-(furan-2-yl)-6- methylthieno[2,3- d]pyrimidine | LCMS: ESI [M + H]⁺ = 251.0 |
| KH 5089 | 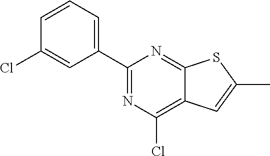 [SMILES: Cc1cc2c(Cl)nc(-c3cccc(Cl)c3)nc2s1] | 1 d (70° C.), 1 d (100° C.) | 1 h (90° C.) | 63% | 4-chloro-2-(3-chlorophenyl)- 6-methylthieno[2,3- d]pyrimidine | LCMS: ESI [M + H]⁺ = 295.0 |
| KH 5090 | 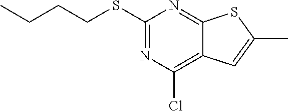 [SMILES: CCCCSc1nc(Cl)c2cc(C)sc2n1] | 1 d (70° C.), 1 d (100° C.) | 1 h (90° C.) | 42% | 2-(butylthio)-4-chloro-6- methylthieno[2,3- d]pyrimidine | LCMS: ESI [M + H]⁺ = 273.0 |
| KH 5091 | 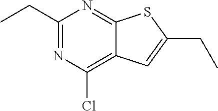 | 1 d (70° C.), 1 d (100° C.) | 2 h (90° C.) | 53% | 4-chloro-2,6- diethylthieno[2,3- d]pyrimidine | LCMS: ESI [M + H]⁺ = 227.1 |
| KH 5093 | 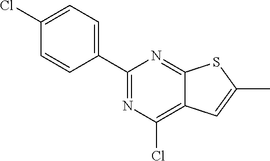 [SMILES: Cc1cc2c(Cl)nc(-c3ccc(Cl)cc3)nc2s1] | 1 d (70° C.), 4 h (100° C.) | 1 h (90° C.) | 76% | 4-chloro-2-(4-chlorophenyl)- 6-methylthieno[2,3- d]pyrimidine | LCMS: ESI [M + H]⁺ = 295.0 |
| KH 5094 | 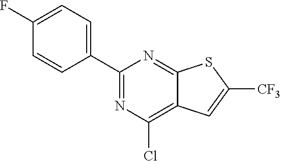 [SMILES: Fc1ccc(-c2nc(Cl)c3cc(C(F)(F)F)sc3n2)cc1] | 1 d (70° C.), 1 d (100° C.) | 1 h (90° C.) | 40% | 4-chloro-2-(4-fluorophenyl)- 6-(trifluoromethyl)thieno[2,3- d]pyrimidine | LCMS: ESI [M + H]⁺ = 333.0 |
| KH 5095 | 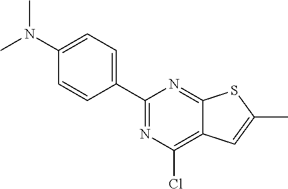 | 1 d (70° C.), 1 d (100° C.) | 1 h (90° C.) | 78% | 4-(4-chloro-6- methylthieno[2,3- d\|pyrimidin-2-yl)-N,N- dimethylaniline | LCMS: ESI [M + H]⁺ = 304.1 |
| KH 5096 | 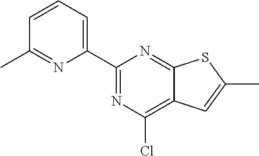 | 1 d (70° C.), 2 h (90° C.) | 2 h (90° C.) | 60% | 4-chloro-6-methyl-2-(6- methylpyridin-2- yl)thieno[2,3-d]pyrimidine | LCMS: ESI [M + H]⁺ = 276.1 |
| KH 6017 | 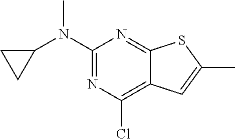 [SMILES: Cc1cc2c(Cl)nc(N(C)C3CC3)nc2s1] | 1 d (70° C.), 1 d (100° C.), 1 d (110° C.) | 1 h (90° C.) | 58% | 4-chloro-N-cyclopropyl-N,6- dimethylthieno[2,3- d]pyrimidin-2-amine | LCMS: ESI [M + H]⁺ = 254.1 |
| KH 6018 | 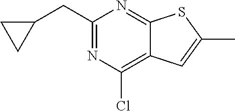 | 1 d (70° C.), 4 h (110° C.) | 1 h (90° C.) | 61% | 4-chloro-2- (cyclopropylmethyl)-6- methylthieno[2,3- d]pyrimidine | LCMS: ESI [M + H]⁺ = 239.1 |
| KH 6019 | 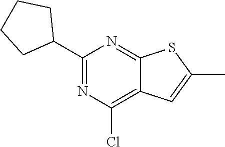 [SMILES: Cc1cc2c(Cl)nc(C3CCCC3)nc2s1] | 1 d (70° C.), 4 h (110° C.) | 1 h (90° C.) | 61% | 4-chloro-2-cyclopentyl-6- methylthieno[2,3- d]pyrimidine | LCMS: ESI [M + H]⁺ = 253.1 |
| KH 6020 | 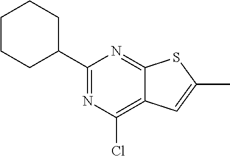 [SMILES: Cc1cc2c(Cl)nc(C3CCCCC3)nc2s1] | 1 d (70° C.), 1 d (110° C.) | 1 h (90° C.) | 69% | 4-chloro-2-cyclohexyl-6- methylthieno[2,3- d]pyrimidine | LCMS: ESI [M + H]⁺ = 267.1 |
| KH 6029 | 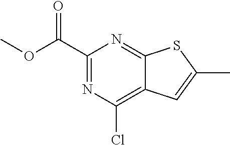 | 1 d (70° C.) | 1 h (90° C.) | 32% | methyl 4-chloro-6- methylthieno[2,3- d]pyrimidine-2-carboxylate | LCMS: ESI [M + H]⁺ = 243.0 |
| KH 6061 | 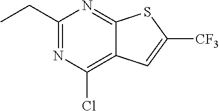 [SMILES: CCc1nc(Cl)c2cc(C(F)(F)F)sc2n1] | 1 d (70° C.), 1 d (100° C.) | 1 h (90° C.) | 16% | 4-chloro-2-ethyl-6- (trifluoromethyl)thieno[2,3- d]pyrimidine | LCMS: ESI [M + H]⁺ = 267.0 |
| KH 6065 | 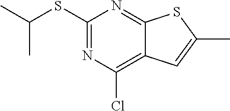 | 1 d (70° C.), 3 d (100° C.) | 1 h (90° C.) | 38% | 4-chloro-2-(isopropylthio)-6- methylthieno[2,3- d]pyrimidine | LCMS: ESI [M + H]⁺ = 259.0 |
| KH 6066 | 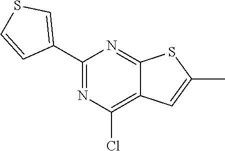 [SMILES: Cc1cc2c(Cl)nc(-c3ccsc3)nc2s1] | 1 d (70° C.), 1 d (100° C.) | 1 h (90° C.) | 75% | 4-chloro-6-methyl-2- (thiophen-3-yl)thieno[2,3- d]pyrimidine | LCMS: ESI [M + H]⁺ = 267.0 |
| KH 6067 | 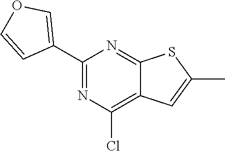 [SMILES: Cc1cc2c(Cl)nc(-c3ccoc3)nc2s1] | 1 d (70° C.), 1 d (100° C.) | 1 h (90° C.) | 28% | 4-chloro-2-(furan-3-yl)-6- methylthieno[2,3- d]pyrimidine | LCMS: ESI [M + H]⁺ = 251.0 |
| KH 6089 | 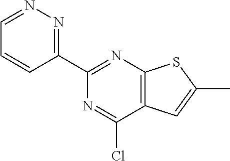 [SMILES: Cc1cc2c(Cl)nc(-c3cccnn3)nc2s1] | 1 d (70° C.) | 1 h (90° C.) | 65% | 4-chloro-6-methyl-2- (pyridazin-3-yl)thieno[2,3- d]pyrimidine | LCMS: ESI [M + H]⁺ = 263.0 |
| KH 6090 | 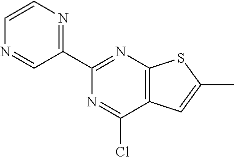 [SMILES: Cc1cc2c(Cl)nc(-c3cnccn3)nc2s1] | 1 d (70° C.) | 1 h (90° C.) | 79% | 4-chloro-6-methyl-2-(pyrazin- 2-yl)thieno[2,3-d]pyrimidine | LCMS: ESI [M + H]⁺ = 263.0 |
| KH 6091 | 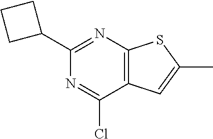 | 1 d (70° C.), 1 d (90° C.) | 1 h (90° C.) | 63% | 4-chloro-2-cyclobutyl-6- methylthieno[2,3- d]pyrimidine | LCMS: ESI [M + H]⁺ = 239.0 |
| KH 6092 | 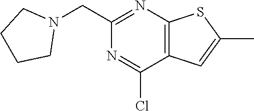 [SMILES: Cc1cc2c(Cl)nc(CN3CCCC3)nc2s1] | 1 d (70° C.) | 1 h (90° C.) | 19% | 4-chloro-6-methyl-2- (pyrrolidin-1- ylmethyl)thieno[2,3- d]pyrimidine | LCMS: ESI [M + H]⁺ = 268.1 |
| KH 6093 | 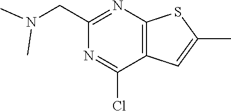 | 1 d (70° C.) | 1 h (90° C.) | 70% | 1-(4-chloro-6- methylthieno[2,3- d]pyrimidin-2-yl)-N,N- dimethylmethanamine | LCMS: ESI [M + H]⁺ = 242.1 |
| KH 7018 | 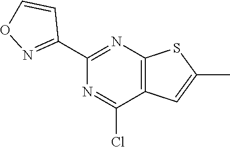 [SMILES: Cc1cc2c(Cl)nc(-c3ccon3)nc2s1] | 3 d (70° C.) | 30 m (90° C.) | 81% | 3-(4-chloro-6- methylthieno[2,3- d\|pyrimidin-2-yl)isoxazole | LCMS: ESI [M + H]⁺ = 252.1 |
| KH 7019 | 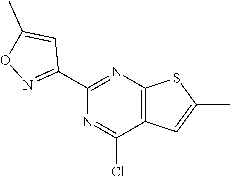 [SMILES: Cc1cc(-c2nc(Cl)c3cc(C)sc3n2)no1] | 3 d (70° C.) | 30 m (90° C.) | 72% | 3-(4-chloro-6- methylthieno[2,3- d]pyrimidin-2-yl)-5- methylisoxazole | LCMS: ESI [M + H]⁺ = 266.0 |
| KH 7020 | 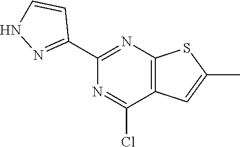 [SMILES: Cc1cc2c(Cl)nc(-c3cc[nH]n3)nc2s1] | 3 d (70° C.) | 1 d (85° C.) | 78% | 4-chloro-6-methyl-2-(1H- pyrazol-3-yl)thieno[2,3- d]pyrimidine | LCMS: ESI [M + H]⁺ = 251.0 |
| KH 7033 | 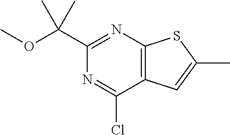 | 1 d (70° C.), 1 d (90° C.) | 1 h (90° C.) | 45% | 4-chloro-2-(2- methoxypropan-2-yl)-6- methylthieno[2,3- d]pyrimidine | LCMS: ESI [M + H]⁺ = 257.1 |
| KH 7048 | 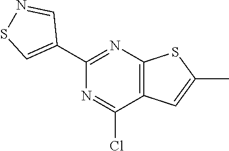 [SMILES: Cc1cc2c(Cl)nc(-c3cnsc3)nc2s1] | 3 d (70° C.), 1 h (90° C.), 1 d (100° C.) | 2 h (90° C.) | 64% | 4-chloro-2-(isothiazol-4-yl)- 6-methylthieno[2,3- d]pyrimidine | LCMS: ESI [M + H]⁺ = 268.0 |
| KH 7049 | 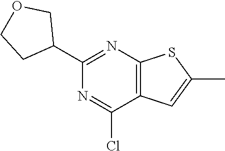 [SMILES: Cc1cc2c(Cl)nc(C3CCOC3)nc2s1] | 6 h (70° C.), 1 d (100° C.) | 2 h (90° C.) | 30% | 4-chloro-6-methyl-2- (tetrahydrofuran-3- yl)thieno[2,3-d]pyrimidine | LCMS: ESI [M + H]⁺ = 255.1 |
| KH 7076 | 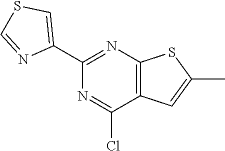 [SMILES: Cc1cc2c(Cl)nc(-c3cscn3)nc2s1] | 1 d (70° C.), 4 h (90° C.) | 1 h (90° C.) | 71% | 4-chloro-6-methyl-2-(thiazol- 4-yl)thieno[2,3-d]pyrimidine | LCMS: ESI [M + H]⁺ = 268.0 |
| KH 7077 | 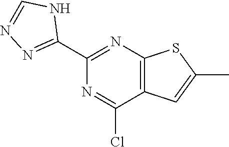 [SMILES: Cc1cc2c(Cl)nc(-c3nnc[nH]3)nc2s1] | 1 d (70° C.) | 1 h (90° C.) | 92% | 4-chloro-6-methyl-2-(4H- 1,2,4-triazol-3-yl)thieno[2,3- d]pyrimidine | LCMS: ESI [M + H]⁺ = 252.0 |
| KH 7078 | 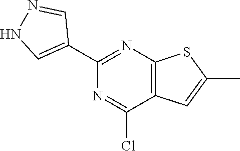 [SMILES: Cc1cc2c(Cl)nc(-c3cn[nH]c3)nc2s1] | 1 d (70° C.), 5 h (90° C.), 1 d (100° C.) | 2 h (90° C.) | 65% | 4-chloro-6-methyl-2-(1H- pyrazol-4-yl)thieno[2,3- d]pyrimidine | LCMS: ESI [M + H]⁺ = 251.0 |
| KH 7079 | 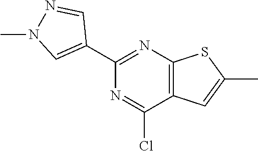 | 1 d (70° C.), 5 h (90° C.), 1 d (100° C.) | 6 h (90° C.), 1 d (100° C.) | 66% | 4-chloro-6-methyl-2-(1- methyl-1H-pyrazol-4- yl)thieno[2,3-d]pyrimidine | LCMS: ESI [M + H]⁺ = 265.0 |
| KH 7080 | 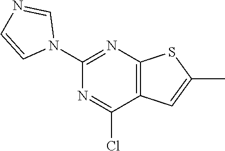 [SMILES: Cc1cc2c(Cl)nc(-n3ccnc3)nc2s1] | 1 d (70° C.) | 1 h (90° C.) | 18% | 4-chloro-2-(1H-imidazol-1- yl)-6-methylthieno[2,3- d]pyrimidine | LCMS: ESI [M + H]⁺ = 251.1 |
| KH 7081 | 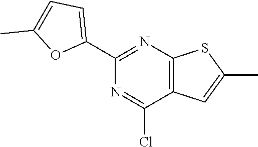 | 1 d (70° C.), 5 h (90° C.), 1 d (100° C.) | 1 h (90° C.) | 71% | 4-chloro-6-methyl-2-(5- methylfuran-2-yl)thieno[2,3- d]pyrimidine | LCMS: ESI [M + H]⁺ = 265.0 |
| KH 8005 | 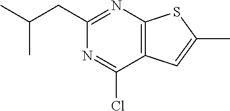 | 1 d (70° C.), 1 d (100° C.) | 2 h (90° C.) | 43% | 4-chloro-2-isobutyl-6- methylthieno[2,3- d]pyrimidine | LCMS: ESI [M + H]⁺ = 241.1 |
| KH 8019 | 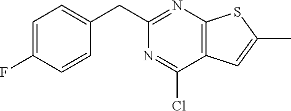 [SMILES: Cc1cc2c(Cl)nc(Cc3ccc(F)cc3)nc2s1] | 1 d (70° C.), 1 d (100° C.) | 8 h (100° C.) | 56% | 4-chloro-2-(4-fluorobenzyl)- 6-methylthieno[2,3- d]pyrimidine | LCMS: ESI [M + H]⁺ = 293.1 |
| KH 8020 | 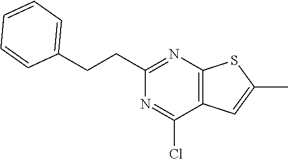 [SMILES: Cc1cc2c(Cl)nc(CCc3ccccc3)nc2s1] | 1 d (70° C.), 1 d (100° C.) | 1 h (100° C.) | 53% | 4-chloro-6-methyl-2- phenethylthieno[2,3- d]pyrimidine | LCMS: ESI [M + H]⁺ = 289.1 |
| KH 8024 | 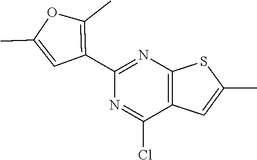 | 1 d (70° C.), 1 d (100° C.) | 1 h (90° C.) | 35% | 4-chloro-2-(2,5- dimethylfuran-3-yl)-6- methylthieno[2,3- d]pyrimidine | LCMS: ESI [M + H]⁺ = 279.0 |
| KH 8029 | 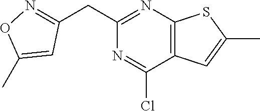 | 1 d (70° C.), 1 d (100° C.) | 1 h (100° C.) | 61% | 3-((4-chloro-6- methylthieno[2,3- d]pyrimidin-2-yl)methyl)-5- methylisoxazole | LCMS: ESI [M + H]⁺ = 280.1 |
| KH 8030 | 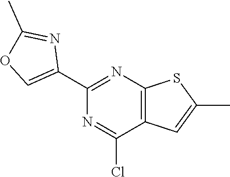 [SMILES: Cc1nc(-c2nc(Cl)c3cc(C)sc3n2)co1] | 1 d (70° C.), 1 d (100° C.) | 30 m (90° C.) | 15% | 4-(4-chloro-6- methylthieno[2,3- d]pyrimidin-2-yl)-2- methyloxazole | LCMS: ESI [M + H]⁺ = 266.1 |
| KH 8031 | 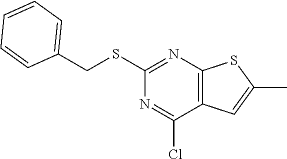 [SMILES: Cc1cc2c(Cl)nc(SCc3ccccc3)nc2s1] | 1 d (70° C.), 1 d (100° C.) | 20 m (90° C.) | 48% | 2-(benzylthio)-4-chloro-6- methylthieno[2,3- d]pyrimidine | LCMS: ESI [M + H]⁺ = 307.0 |
| KH 8054 | 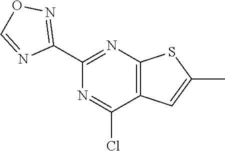 [SMILES: Cc1cc2c(Cl)nc(-c3ncon3)nc2s1] | 1 h (25° C.), 1 d (50° C.) | 2 h (90° C.) | 28% | 3-(4-chloro-6- methylthieno[2,3- d]pyrimidin-2-yl)-1,2,4- oxadiazole | LCMS: ESI [M + H]⁺ = 253.1 |
| KH 8088 | 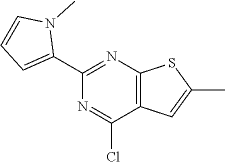 [SMILES: Cc1cc2c(Cl)nc(-c3cccn3C)nc2s1] | 1 d (70° C.) | 1 h (90° C.) | 25% | 4-chloro-6-methyl-2-(1- methyl-1H-pyrrol-2- yl)thieno[2,3-d]pyrimidine | LCMS: ESI [M + H]⁺ = 264.0 |
| KH 8098 | 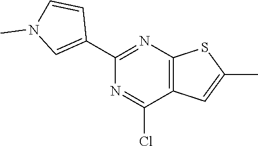 | 1 d (70° C.), 1 d (110° C.) | 4 h (90° C.) | 29% | 4-chloro-6-methyl-2-(1- methyl-1H-pyrrol-3- yl)thieno[2,3-d]pyrimidine | LCMS: ESI [M + H]⁺ = 264.0 |
| KH 8099 | 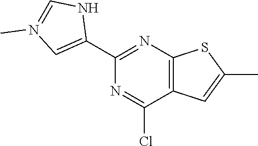 | 1 d (70° C.), 1 d (110° C.) | 1 d (80° C.) | 99% | 4-chloro-2-(1H-imidazol-5- yl)-6-methylthieno[2,3- d]pyrimidine | LCMS: ESI [M + H]⁺ = 251.1 |
| KH 9003 | 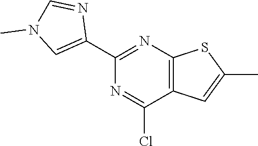 | 1 d (70° C.) | 1 d (80° C.) | 79% | 4-chloro-6-methyl-2-(1- methyl-1H-imidazol-4- yl)thieno[2,3-d]pyrimidine | LCMS: ESI [M + H]⁺ = 265.0 |
| KH 9004 | 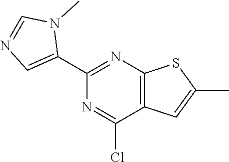 [SMILES: Cc1cc2c(Cl)nc(-c3cncn3C)nc2s1] | 1 d (70° C.) | 8 h (95° C.) | 41% | 4-chloro-6-methyl-2-(1- methyl-1H-imidazol-5- yl)thieno[2,3-d]pyrimidine | LCMS: ESI [M + H]⁺ = 265.0 |
| KH 9078 | 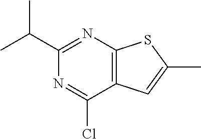 | 1 d (70° C.), 1 d (100° C.) | 30 m (90° C.) | 58% | 4-chloro-2-isopropyl-6- methylthieno[2,3- d]pyrimidine | LCMS: ESI [M + H]⁺ = 227.1 |
| KH 9091 | 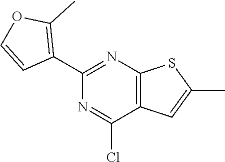 [SMILES: Cc1cc2c(Cl)nc(-c3ccoc3C)nc2s1] | 1 d (70° C.), 1 d (100° C.) | 1 h (90° C.) | 68% | 4-chloro-6-methyl-2-(2- methylfuran-3-yl)thieno[2,3- d]pyrimidine | LCMS: ESI [M + H]⁺ = 265.1 |
| KH 9100 | 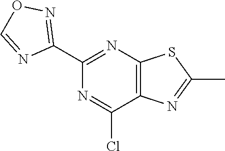 [SMILES: Cc1nc2c(Cl)nc(-c3ncon3)nc2s1] | 1 d (35° C.), 2 d (45° C.) | 2 h (90° C.) | 4% | 3-(7-chloro-2- methylthiazolo[5,4- d]pyrimidin-5-yl)-1,2,4- oxadiazole | LCMS: ESI [M + H]⁺ = 254.1 |
| KH 11057 | 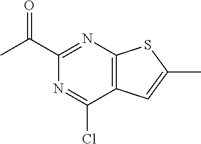 | 5 d (50° C.) | 30 m (25° C.), 30 m (70° C.) | 18% | 1-(4-chloro-6- methylthieno[2,3- d]pyrimidin-2-yl)ethan-1-one | LCMS: ESI [M + H]⁺ = 227.1 |
| KH 11064 | 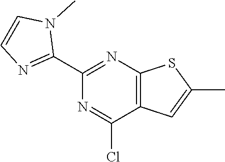 [SMILES: Cc1cc2c(Cl)nc(-c3nccn3C)nc2s1] | 2 h (50° C.), 1 d (70° C.) | 1 h (90° C.) | 38% | 4-chloro-6-methyl-2-(1- methyl-1H-imidazol-2- yl)thieno[2,3-d]pyrimidine | LCMS: ESI [M + H]⁺ = 265.0 |
| KH 10034 | 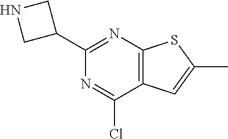 | 1 d (25° C.), 1 d (50° C.) | 1 h (90° C.) | 38% | 2-(azetidin-3-yl)-4-chloro-6- methylthieno[2,3- d]pyrimidine | LCMS: ESI [M + H]⁺ = 240.1 |
| KH 11067 | 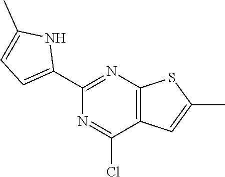 [SMILES: Cc1ccc(-c2nc(Cl)c3cc(C)sc3n2)[nH]1] | 1 d (70° C.), 3 d (100° C.), 1 d (110° C.), 1 d (120° C.) | 1 h (90° C.) | 93% | 4-chloro-6-methyl-2-(5- methyl-1H-pyrrol-2- yl)thieno[2,3-d]pyrimidine | LCMS: ESI [M + H]⁺ = 264.1 |

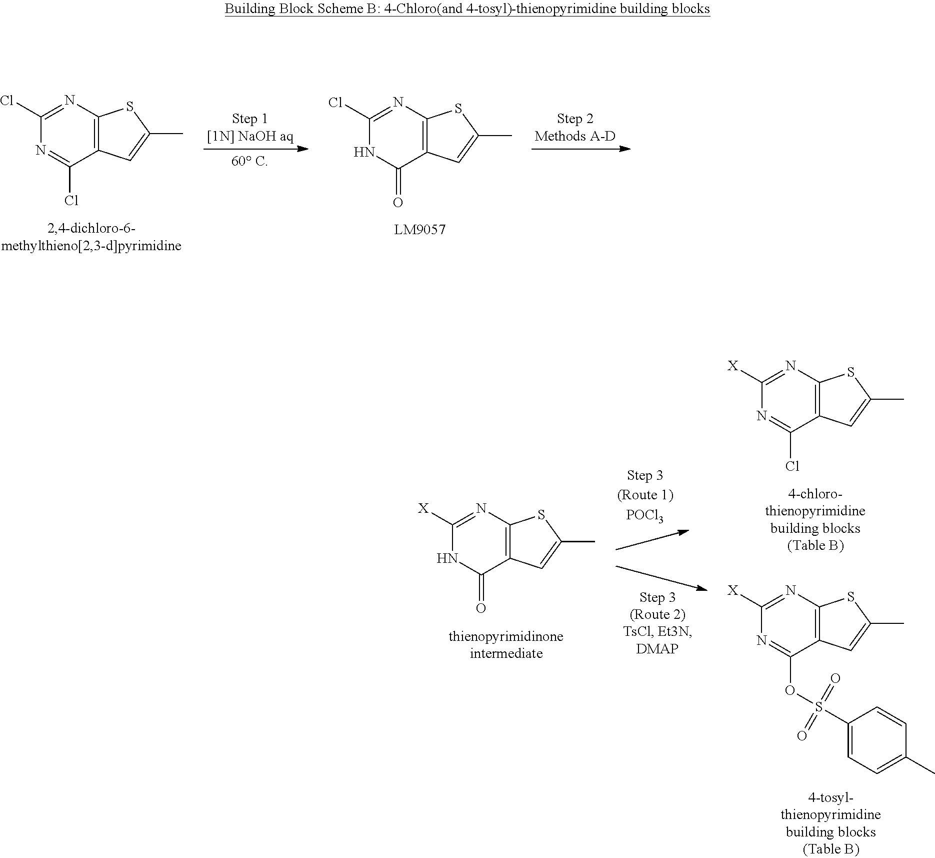

General Procedure Scheme B: Reaction conditions and compound data for specific examples are listed in Table B. The starting material was commercially purchased.

Step 1: 2,4-dichloro-6-methylthieno[2,3-d]pyrimidine (484.0 mg, 2.2 mmol) was dissolved in THF (10 mL) and [1N] NaOH(aq) (5.5 mL) was added. The reaction was stirred at 60° C. for 5 hours. Upon completion, the reaction was cooled to room temperature and acidified to pH ˜2 with [4N] HCl(aq). The product was filtered off and washed well with DI water, then dried in vacuo, to give intermediate LM9057: 84% yield; IUPAC: 2-chloro-6-methylthieno[2,3-d]pyrimidin-4(3H)-one; LCMS: ESI [M+H]⁺=201.1.

Step 2 (Method A): To a threaded pressure vial was added the desired sodium alkoxide solution (NaOR) (1 mL) specified (conditions in Table B) (if not commercially available, a [2M] NaOR solution was made by the careful addition of sodium metal (46 mg, 2.0 mmol) to the desired alcohol ROH (1 mL), and the solution used after the sodium was fully reacted). Intermediate LM9057 (30 mg, 0.15 mmol) was added to the vial, and the reaction was sealed and stirred for the time and temperature specified. Upon completion, the reaction was cooled to room temperature and neutralized with [1N] HCl(aq), concentrated in vacuo, then purified by silica gel column chromatography to give the intermediate thienopyrimidinone for use in step 3.

Step 2 (Method B): To a threaded pressure vial was added DMSO (1 mL) and intermediate LM9057 (30 mg, 0.15 mmol). The desired nucleophile (2-8 eqv) was added, and the reaction was sealed and stirred for the specified time and temperature (conditions Table B). Upon completion, the reaction was cooled to room temperature and concentrated in vacuo. The crude reaction mixture was purified by silica gel column chromatography to give the intermediate thienopyrimidinone for use in Step 3.

Step 2 (Method C): To a threaded pressure vial was added intermediate LM9057 (30 mg, 0.15 mmol), the desired aryl alcohol (0.5 mL) and K₂CO₃ (100 mg, 0.75 mmol). The reaction was sealed and stirred for the specified time and temperature (conditions in Table B). Upon completion, the reaction was cooled to room temperature. DI water was added (5 mL), and the reaction mixture was extracted with DCM (3×). The organic fractions were combined and concentrated in vacuo, and the crude thienopyrimidinone was used without further purification in Step 3.

Step 2 (Method D): Following the General Suzuki Protocol, intermediate LM9047 (1.50 mmol) and (1-(tert-butoxycarbonyl)-1H-pyrrol-2-yl)boronic acid (2.99 mmol) were reacted using the conditions specified in Table B, at which time both the Boc-protected and Boc-deprotected products were detected by LCMS. The reaction was partially purified by silica gel column chromatography and both intermediate products were collected and combined for step 3, whereupon the remaining Boc-protecting groups were cleaved.

Step 3 (Route 1): Following the General POCl₃ Protocol, the thienopyrimidinone intermediate (from step 2) was reacted with POCl₃, for the specified time and temperature to give the desired 4-chloro-thienopyrimidine building block (Table B).

Step 3 (Route 2): To a reaction flask was added the thienopyrimidinone intermediate (from step 2) (0.058 mmol) and DCM (1 mL). Et₃N (1.7 eqv.) was added, followed by 4-toluenesulfonyl chloride (1.5 eqv.) then DMAP (0.05 eqv.), and the reaction was stirred overnight at room temperature. Upon completion, the reaction was concentrated in vacuo (without heating) and purified by silica gel column chromatography to give the 4-tosy-thienopyrimidine building block (Table B).

TABLE B
Building blocks from Scheme B

| Int. | | Step 2 | Step 2 | Step 3 | Step 3 | Yield | | Compound |
|---|---|---|---|---|---|---|---|---|
| No. | Structure | Method | Conditions | Route | Conditions | (2 steps) | IUPAC | data |
| LM 9031 | 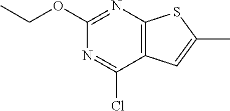 | A | [3M] NaOEt 5 h (80° C.) | 1 | 1.5 h (90° C.) | 70% | 4-chloro-2-ethoxy-6- methylthieno[2,3- d]pyrimidine | LCMS: ESI [M + H]⁺ = 229.0 |
| LM 9038 | 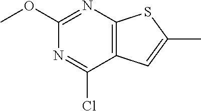 | A | [1M] NaOMe 2 d (70° C.) | 1 | 1 h (70° C.) | 67% | 4-chloro-2-methoxy-6- methylthieno[2,3- d]pyrimidine | LCMS: ESI [M + H]⁺ = 215.0 |
| LM 9044 | 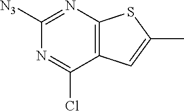 | B | NaN₃ (2 eqv) 2 h (60° C.) | 1 | 1.5 h (90° C.) | 72% | 2-azido-4-chloro-6- methylthieno[2,3- d]pyrimidine | LCMS: ESI [M + H]⁺ = 226.0 |
| LM 9048 | 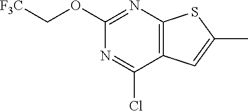 [SMILES: Cc1cc2c(Cl)nc(OCC(F)(F)F)nc2s1] | A | [2M] NaOCH₂CF₃ 1 d (110° C.) | 1 | 2 h (90° C.) | 33% | 4-chloro-6-methyl-2- (2,2,2- trifluoroethoxy)thieno[2,3- d]pyrimidine | LCMS: ESI [M + H]⁺ = 283.0 |
| LM 9061 | 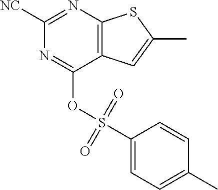 [SMILES: Cc1ccc(S(=O)(=O)Oc2nc(C#N)nc3sc(C)cc23)cc1] | B | KCN (8 eqv) 1 d (150° C.) | 2 | 1 d (25° C.) | 53% | 2-cyano-6- methylthieno[2,3- d]pyrimidin-4-yl 4- methylbenzenesulfonate | LCMS: ESI [M + H]⁺ = 346.1 |
| LM 9074 | 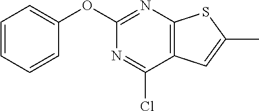 [SMILES: Cc1cc2c(Cl)nc(Oc3ccccc3)nc2s1] | C | K₂CO₃ 1 d (120° C.) | 1 | 1.5 h (90° C.) | 23% | 4-chloro-6-methyl-2- phenoxythieno[2,3- d]pyrimidine | LCMS: ESI [M + H]⁺ = 277.0 |
| LM 9078 | 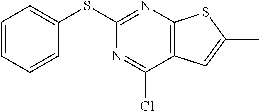 [SMILES: Cc1cc2c(Cl)nc(Sc3ccccc3)nc2s1] | C | K₂CO₃ 2 h (120° C.) | 1 | 1.5 h (90° C.) | 99% | 4-chloro-6-methyl-2- (phenylthio)thieno[2,3- d]pyrimidine | LCMS: ESI [M + H]⁺ = 293.0 |
| LM 16046 | 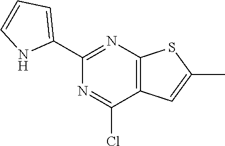 [SMILES: Cc1cc2c(Cl)nc(-c3ccc[nH]3)nc2s1] | D | Cs₂CO₃ 1 h (90° C.) 2 h (70° C.) | 1 | 1.5 h (90° C.) | 49% | 4-chloro-6-methyl-2-(1H- pyrrol-2-yl)thieno[2,3- d]pyrimidine | LCMS: ESI [M + H]⁺ = 250.1 |

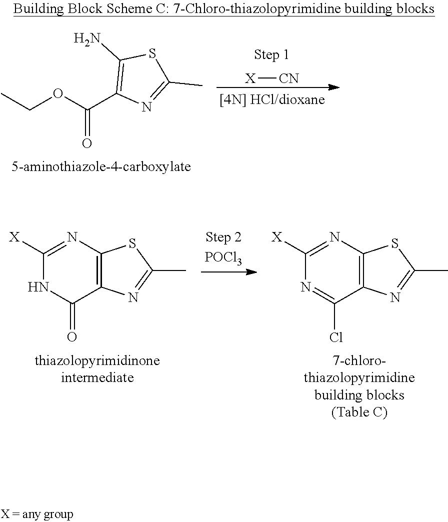

General Procedure Scheme C: Reaction conditions and compound data for specific examples are listed in Table C. The starting material and reagents were commercially purchased.

Step 1: To a threaded pressure vessel, was added 5-aminothiazole-4-carboxylate (0.40 mmol) and X—CN (2 eqv.), followed by the addition of [4N] HCl/dioxane (2 mL). The vial was sealed and heated for the specified time and temperature (conditions in Table C). Upon completion, the reaction was cooled to room temperature and the solvents were evaporated. The crude material was purified by silica gel column chromatography. The thiazolopyrimidinone intermediate was dried under high vacuum overnight for use in step 2.

Step 2: Following the General POCl₃ Protocol, the thiazolopyrimidinone intermediate (from step 1) was reacted with POCl₃, for the specified time and temperature. Note: after quenching, the entire reaction mixture was evaporated for purification by silica gel column chromatography to give the pure 7-chloro-thiazolopyrimidine building block (Table C).

TABLE C
Building blocks from Scheme C

| Int. | | Step 1 | Step 2 | Yield | | Compound |
|---|---|---|---|---|---|---|
| No. | Structure | Conditions | Conditions | (2 steps) | IUPAC | Data |
| LM 8070 | 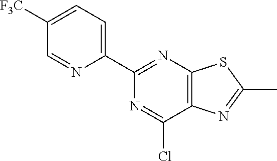 [SMILES: Cc1nc2c(Cl)nc(-c3ccc(C(F)(F)F)cn3)nc2s1] | 16 h (70° C.), 5 h (90° C.) | 2 h (90° C.) | 32% | 7-chloro-2-methyl-5-(5- (trifluoromethyl)pyridin-2- yl)thiazolo[5,4-d]pyrimidine | LCMS: ESI [M + H]⁺ = 331.0 |
| LM 8071 | 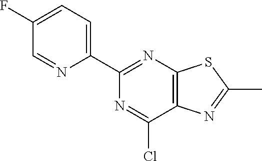 [SMILES: Cc1nc2c(Cl)nc(-c3ccc(F)cn3)nc2s1] | 16 h (70° C.), 5 h (90° C.) | 2 h (90° C.) | 33% | 7-chloro-5-(5-fluoropyridin-2-yl)- 2-methylthiazolo[5,4-d]pyrimidine | LCMS: ESI [M + H]⁺ = 281.0 |
| LM 8073 | 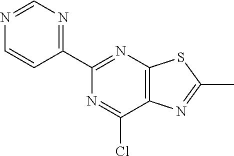 [SMILES: Cc1nc2c(Cl)nc(-c3ccncn3)nc2s1] | 16 h (70° C.) 1 h (90° C.) | 3 h (90° C.) | 11% | 7-chloro-2-methyl-5-(pyrimidin-4- yl)thiazolo[5,4-d]pyrimidine | LCMS: ESI [M + H]⁺ = 264.0 |
| LM 13028 | 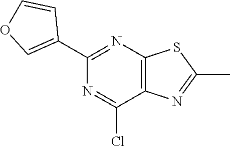 [SMILES: Cc1nc2c(Cl)nc(-c3ccoc3)nc2s1] | 1 d (70° C.), 1 d (80° C.), 1 d (100° C.) | 2 h (95° C.) | 17% | 7-chloro-5-(furan-3-yl)-2- methylthiazolo[5,4-d]pyrimidine | LCMS: ESI [M + H]⁺ = 252.1 |
| LM 13029 | 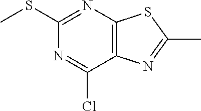 | 1 d (70° C.), 1 d (80° C.), 1 d (100° C.) | 2 h (95° C.) | 11% | 7-chloro-2-methyl-5- (methylthio)thiazolo[5,4- d]pyrimidine | LCMS: ESI [M + H]⁺ = 232.0 |
| LM 15015 | 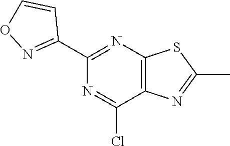 [SMILES: Cc1nc2c(Cl)nc(-c3ccon3)nc2s1] | 1 d (80° C.), 1 d (90° C.), 1 d (100° C.), 4 d (90° C.) | 9 h (100° C.) | 13% | 3-(7-chloro-2-methylthiazolo[5,4- d]pyrimidin-5-yl)isoxazole | LCMS: ESI [M + H]⁺ = 253.1 |

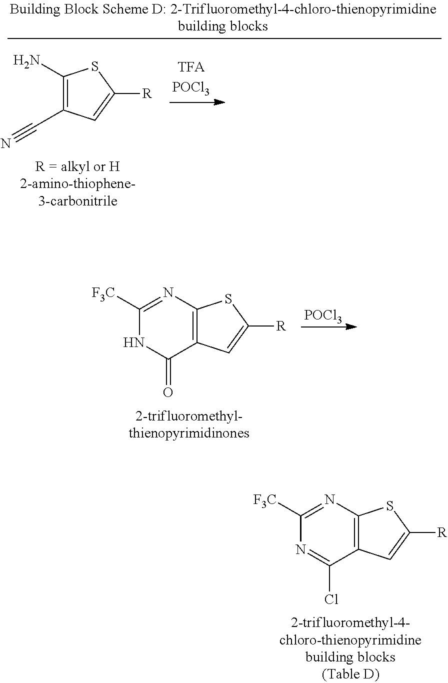

General Procedure Scheme D: Reaction conditions and compound data for specific examples are listed in Table D. Starting materials were commercially purchased.

Step 1: The appropriate commercially available 2-amino-thiophene-3-carbonitrile derivative (1.4 g) was stirred with trifluoroacetic acid (28 mL) at room temperature until the starting material was dissolved. POCl₃ (2.0 mL) was then carefully added and a reflux condenser was attached. The reaction was heated for the specified time and temperature (conditions in Table D), after which time it was cooled room temperature. The POCl₃ and trifluoroacetic acid were removed under reduced pressure, then DI water (10 mL) was slowly added to the remaining orange residue. The aqueous mixture was carefully neutralized with K₂CO₃(s) and the product was extracted with DCM (3×), concentrated in vacuo and dried overnight under high vacuum to give the desired 2-trifluoromethyl-thienopyrimidinone intermediate.

Step 2: Following the General POCl₃ Protocol, the intermediate 2-trifluoromethyl-thienopyrimidinone (from step 1), was reacted with POCl₃ for the specified time and temperature to give the pure 2-trifluoromethyl-4-chloro-thienopyrimidine building block (Table D).

TABLE D
Building blocks from Scheme D

| Int. | | Step 1 | Step 2 | Yield | | |
|---|---|---|---|---|---|---|
| No. | Structure | Conditions | Conditions | (2 steps) | IUPAC | Compound Data |
| LM 13024 F1 | 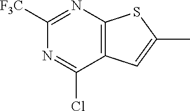 | 1.5 h (60° C.), 30 min 70° C.) | 60 h (110° C.) | 31% | 4-chloro-2- (trifluoromethyl)thieno[2,3- d]pyrimidine | LCMS: ESI [M + H]⁺ = 239.0 |
| KH 6080 | 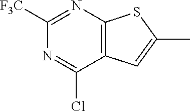 | 30 min (90° C.) | 16 h (100° C.) | 20% | 4-chloro-6-methyl-2- (trifluoromethyl)thieno[2,3- d]pyrimidine | LCMS: ESI [M + H]⁺ = 253.0 |

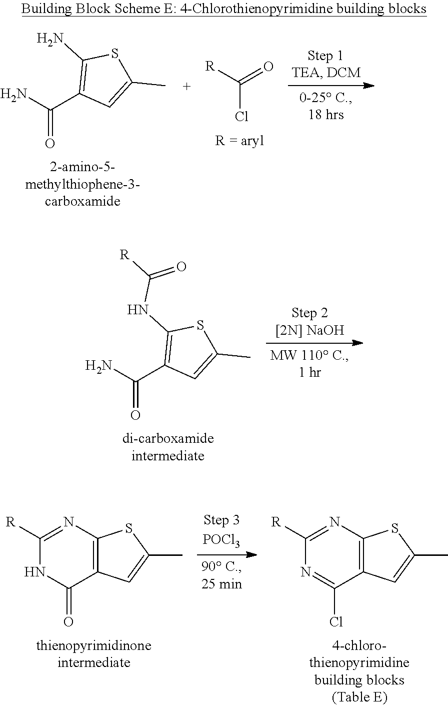

General Procedure for Scheme E: Reaction conditions and compound data for specific examples are listed in Table E. Starting materials and reagents were commercially purchased.

Step 1: 2-amino-5-methylthiophene-3-carboxamide (156 mg, 1.00 mmol) was reacted with appropriate acid chloride (145 mg, 1.00 mmol) in anhydrous DCM (3.0 mL) with Et₃N (139 μL, 1.00 mmol) on an ice bath. The reaction was allowed to slowly reach room temperature and stirred overnight. Upon completion, the reaction was concentrated and purified by silica gel column chromatography to give the corresponding di-carboxamide intermediate for use in step 2.

Step 2: The di-carboxamide intermediate, was heated with [2N] NaOH(aq) (3.0 mL) in a microwave reactor at 100° C. for 1 hour. The reaction was acidified with the addition of [1N] HCl(aq) and filtered to collect the thienopyrimidinone intermediate as a solid which was dried under high vacuum before use in step 3.

Step 3: Following the General POCl₃ Protocol, the intermediate thienopyrimidinone, was reacted with POCl₃ for 25 minutes at 90° C. to give the pure 4-chloro-thienopyrimidine building block (Table E).

TABLE E
Building blocks from Scheme E

| Int. | | Yield | | |
|---|---|---|---|---|
| No | Structure | (3 steps) | IUPAC | Compound Data |
| KH 9049 | 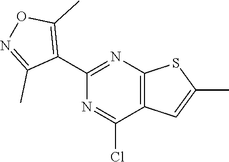 [SMILES: Cc1cc2c(Cl)nc(-c3c(C)noc3C)nc2s1] | 16% | 4-(4-chloro-6-methylthieno[2,3- d]pyrimidin-2-yl)-3,5-dimethylisoxazole | LCMS: ESI [M + H]⁺ = 281.0 |
| KH 9051 | 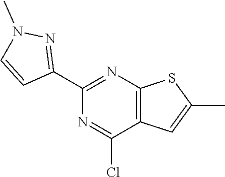 [SMILES: Cc1cc2c(Cl)nc(-c3ccn(C)n3)nc2s1] | 10% | 4-chloro-6-methyl-2-(1-methyl-1H- pyrazol-3-yl)thieno[2,3-d]pyrimidine | LCMS: ESI [M + H]⁺ = 265.1 |

### Building Block KH6094

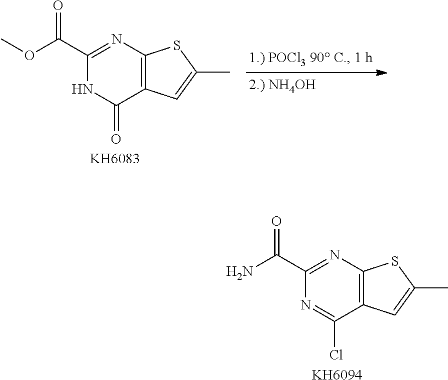

Starting material KH6083 was prepared as described in the synthesis of KH6029 (Scheme A, step 1). Following the General POCl₃ Protocol, KH6083 (83 mg, 0.37 mmol) was reacted with POCl₃ (1.0 mL) and the reaction was heated at 90° C. for 1 hour. During neutralization with 30% NH₄₀H(aq), the temperature of the reaction mixture was not kept cooled (<25° C.), which resulted in conversion of ester to the amide. The amide product was the extracted into DCM (3×) and dried under high vacuum to give building block KH6094: 67% yield; IUPAC: 4-chloro-6-methylthieno[2,3-d]pyrimidine-2-carboxamide; LCMS: ESI [M+H]⁺=228.0.

### Building Block KH8006

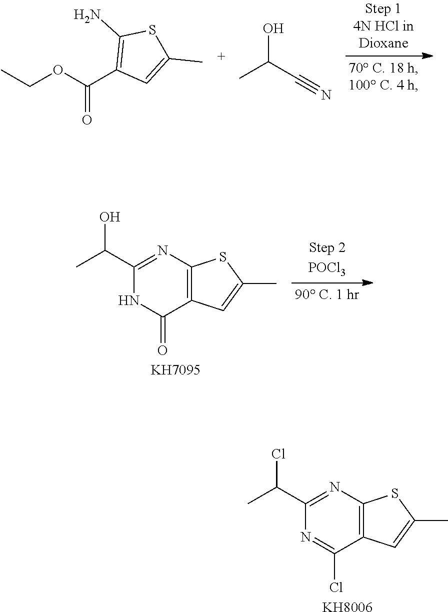

Step 1: Following the general procedure in Scheme A, step 1, commercially available ethyl 2-amino-5-methylthiophene-3-carboxylate (200 mg, 1.08 mmol) was reacted with 2-hydroxypropanenitrile (108 mg, 1.52 mmol) in 4N HCl/dioxane (3.0 mL) at 70° C. for 18 hours then 100° C. for 4 hours to give intermediate KH7095: 86% yield; IUPAC: 2-(1-hydroxyethyl)-6-methylthieno[2,3-d]pyrimidin-4(3H)-one; LCMS: ESI [M+H]⁺=211.1.

Step 2: Following the General POCl₃ Protocol; intermediate KH7095 (195 mg, 0.93 mmol) was reacted with POCl₃ (1.5 mL) for 1 hour at 90° C. to give building block KH8006: 53% yield; IUPAC: 4-chloro-2-(1-chloroethyl)-6-methylthieno[2,3-d]pyrimidine; LCMS: ESI [M+H]⁺=247.0 and 249.0.

### Building Block KH11059

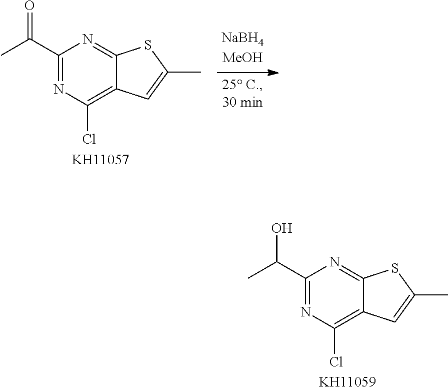

Starting material, building block KH11057 was prepared as described in Scheme A. KH11057 (29 mg, 0.13 mmol) was dissolved in anhydrous MeOH (1.5 mL) before the addition of NaBH4 (6.0 mg, 0.16 mmol). The reaction was attached to a bubbler and allowed to react at room temperature for 30 minutes. The reaction mixture was dried to a solid in vacuo and purified with silica chromatography to obtain building block KH11059: 67% yield; IUPAC: 1-(4-chloro-6-methylthieno[2,3-d]pyrimidin-2-yl)ethan-1-ol; LCMS: ESI [M+H]⁺=229.1.

### Example 3: Scheme 1 SnAr Reaction

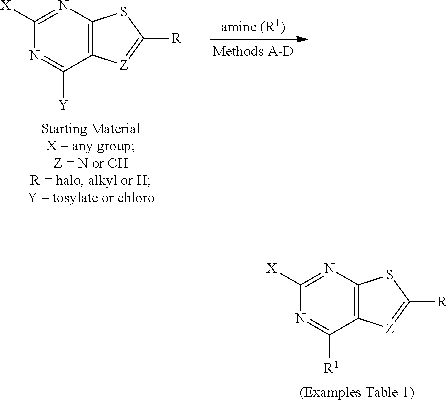

General Procedure for Scheme 1: Reaction conditions and compound data for specific examples are listed in Table 1. Starting materials were synthesized as detailed in the building blocks section, unless otherwise stated.

Method A: The appropriate starting material (0.056) was dissolved in dioxane (1.0 mL) and DIPEA (4 eqv) and the desired commercially available amine (R¹, 2 eqv) was added, and the solution was reacted using the conditions specified. Upon completion, the reaction was concentrated in vacuo, and purified by prep HPLC or silica gel column chromatography, or a combination of the two, to give the desired product (Table 1).

Method B: The appropriate starting material (0.042 mmol) was dissolved in THF (1.0 mL) and triethylamine (3 eqv). The desired commercially available amine (R¹, 1.2 eqv) was added, and the solution was reacted using the conditions specified. Upon completion, the reaction was concentrated in vacuo, and purified by prep HPLC or silica gel column chromatography, or a combination of the two, to give the desired product (Table 1).

Method C: Several aryl-propylamine groups (R¹) were not commercially available, and were synthesized via the scheme below. Aryl-propylamines (R¹) were used crude, and were coupled with the appropriate starting material following the procedure in Method A, to give the desired product (Table 1).

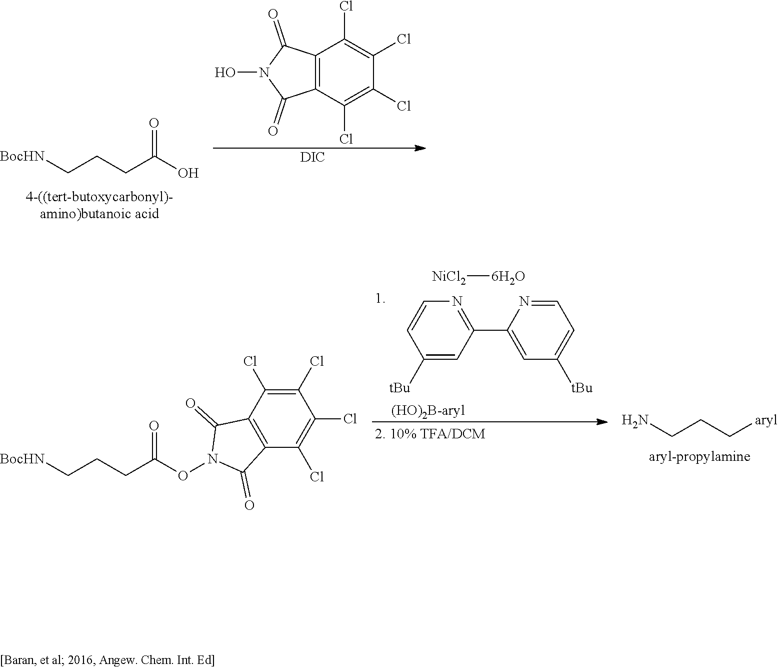

Method D: The appropriate starting material (0.056) was dissolved in DMSO (2.0 mL) and DIPEA (4 eqv). The desired commercially available amine (R¹, 2 eqv) was added, and the solution was reacted using the conditions specified. Upon completion, the reaction was cooled to room temperature and H₂O (0.5 mL) was added. The solid precipitate was filtered (or in the case no precipitate formed, the reaction was extracted with Et₂O and concentrated in vacuo) and purified by prep HPLC or silica gel column chromatography, or a combination of the two, to give the pure product (Table 1).

TABLE 1
Examples from Scheme 1 (Methods A-D)

| Cmpd. | Structure | Starting | Method/ | | |
|---|---|---|---|---|---|
| No. | | Material | Conditions | Yield | IUPAC |
| 1 | 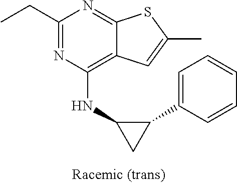 [SMILES: CCc1nc(N[C@@H]2C[C@H]2c2ccccc2)c2cc(C)sc2n1] | KH 4007 | Method A 1 h (140° C.) microwave | 29% | 2-ethyl-6-methyl-N- ((rac-1S,2R)-2- phenylcyclopropyl) thieno[2,3- d]pyrimidin-4-amine |
| 2 | 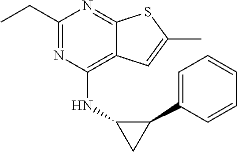 [SMILES: CCc1nc(N[C@H]2C[C@@H]2c2ccccc2)c2cc(C)sc2n1] | KH 4007 | Method A 1 h (140° C.) microwave | 40% | 2-ethyl-6-methyl-N- ((1S,2R)-2- phenylcyclopropyl) thieno[2,3- d]pyrimidin-4-amine |
| 3 | 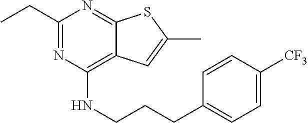 [SMILES: CCc1nc(NCCCc2ccc(C(F)(F)F)cc2)c2cc(C)sc2n1] | KH 4007 | Method A 1 h (140° C.) microwave | 14% | 2-ethyl-6-methyl-N- (3-(4- (trifluoromethyl) phenyl)propyl)thieno [2,3-d]pyrimidin-4- amine |
| 4 | 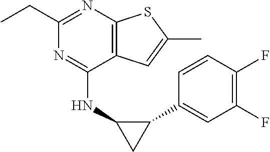 [SMILES: CCc1nc(N[C@@H]2C[C@H]2c2ccc(F)c(F)c2)c2cc(C)sc2n1] | KH 4007 | Method A 1 h (160° C.) microwave | 12% | N-((1R,2S)-2-(3,4- difluorophenyl) cyclopropyl)-2-ethyl-6- methylthieno[2,3- d]pyrimidin-4-amine |
| 5 | 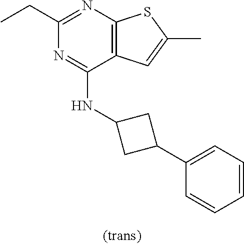 [SMILES: CCc1nc(NC2CC(c3ccccc3)C2)c2cc(C)sc2n1] | KH 4007 | Method A 4 h (140° C.) microwave | 47% | 2-ethyl-6-methyl-N- (3- phenylcyclobutyl) thieno[2,3-d]pyrimidin- 4-amine |
| 6 | 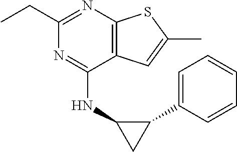 [SMILES: CCc1nc(N[C@@H]2C[C@H]2c2ccccc2)c2cc(C)sc2n1] | KH 4007 | Method A 4 h (140° C.) microwave | 15% | 2-ethyl-6-methyl-N- ((1R,2S)-2- phenylcyclopropyl) thieno[2,3- d]pyrimidin-4-amine |
| 7 | 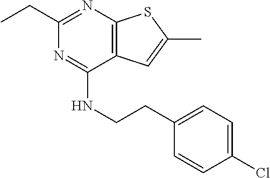 [SMILES: CCc1nc(NCCc2ccc(Cl)cc2)c2cc(C)sc2n1] | KH 4007 | Method A 2 h (140° C.) microwave | 53% | N-(4- chlorophenethyl)-2- ethyl-6- methylthieno[2,3- d]pyrimidin-4-amine |
| 8 | 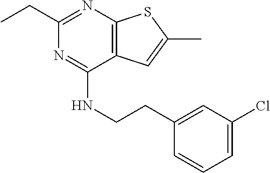 [SMILES: CCc1nc(NCCc2cccc(Cl)c2)c2cc(C)sc2n1] | KH 4007 | Method A 2 h (140° C.) microwave | 23% | N-(3- chlorophenethyl)-2- ethyl-6- methylthieno[2,3- d]pyrimidin-4-amine |
| 9 | 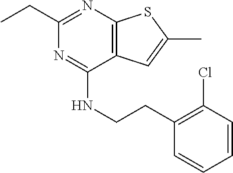 [SMILES: CCc1nc(NCCc2ccccc2Cl)c2cc(C)sc2n1] | KH 4007 | Method A 2 h (140° C.) microwave | 68% | N-(2- chlorophenethyl)-2- ethyl-6- methylthieno[2,3- d]pyrimidin-4-amine |
| 10 | 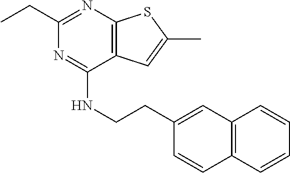 [SMILES: CCc1nc(NCCc2ccc3ccccc3c2)c2cc(C)sc2n1] | KH 4007 | Method A 2 h (140° C.) microwave | 39% | 2-ethyl-6-methyl-N- (2-(naphthalen-2- yl)ethyl)thieno[2,3- d]pyrimidin-4-amine |
| 11 | 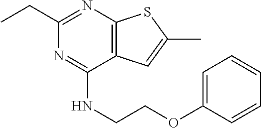 [SMILES: CCc1nc(NCCOc2ccccc2)c2cc(C)sc2n1] | KH 4007 | Method A 2 h (140° C.) microwave | 57% | 2-ethyl-6-methyl-N- (2- phenoxyethyl)thieno [2,3-d]pyrimidin-4- amine |
| 12 | 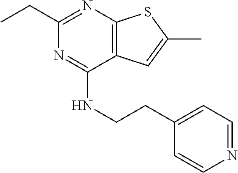 [SMILES: CCc1nc(NCCc2ccncc2)c2cc(C)sc2n1] | KH 4007 | Method A 2 h (140° C.) microwave | 29% | 2-ethyl-6-methyl-N- (2-(pyridin-4- yl)ethyl)thieno[2,3- d]pyrimidin-4-amine |
| 13 | 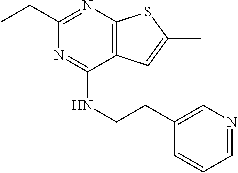 [SMILES: CCc1nc(NCCc2cccnc2)c2cc(C)sc2n1] | KH 4007 | Method A 2 h (140° C.) microwave | 54% | 2-ethyl-6-methyl-N- (2-(pyridin-3- yl)ethyl)thieno[2,3- d]pyrimidin-4-amine |
| 14 | 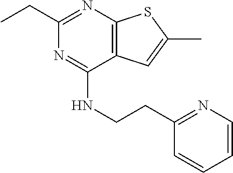 [SMILES: CCc1nc(NCCc2ccccn2)c2cc(C)sc2n1] | KH 4007 | Method A 2 h (140° C.) microwave | 52% | 2-ethyl-6-methyl-N- (2-(pyridin-2- yl)ethyl)thieno[2,3- d]pyrimidin-4-amine |
| 15 | 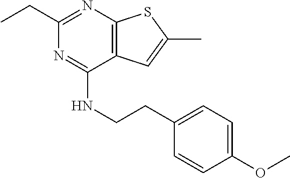 [SMILES: CCc1nc(NCCc2ccc(OC)cc2)c2cc(C)sc2n1] | KH 4007 | Method A 2 h (140° C.) microwave | 66% | 2-ethyl-N-(4- methoxyphenethyl)- 6-methylthieno[2,3- d]pyrimidin-4-amine |
| 16 | 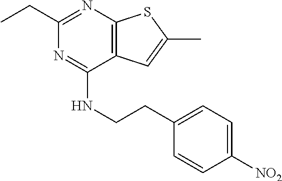 [SMILES: CCc1nc(NCCc2ccc([N+](=O)[O-])cc2)c2cc(C)sc2n1] | KH 4007 | Method A 2 h (140° C.) microwave | 52% | 2-ethyl-6-methyl-N- (4- nitrophenethyl)thieno [2,3-d]pyrimidin-4- amine |
| 17 | 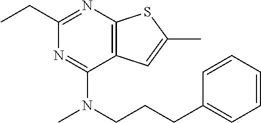 [SMILES: CCc1nc(N(C)CCCc2ccccc2)c2cc(C)sc2n1] | KH 4007 | Method A 2 h (140° C.) microwave | 74% | 2-ethyl-N,6- dimethyl-N-(3- phenylpropyl)thieno [2,3-d]pyrimidin-4- amine |
| 18 | 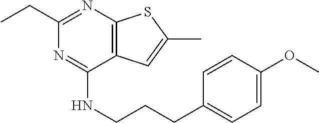 [SMILES: CCc1nc(NCCCc2ccc(OC)cc2)c2cc(C)sc2n1] | KH 4007 | Method A 2 h (140° C.) microwave | 80% | 2-ethyl-N-(3-(4- methoxyphenyl) propyl)-6- methylthieno[2,3- d]pyrimidin-4-amine |
| 19 | 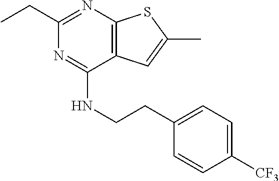 [SMILES: CCc1nc(NCCc2ccc(C(F)(F)F)cc2)c2cc(C)sc2n1] | KH 4007 | Method A 2 h (140° C.) microwave | 52% | 2-ethyl-6-methyl-N- (4- (trifluoromethyl) phenethyl)thieno[2,3- d]pyrimidin-4-amine |
| 20 | 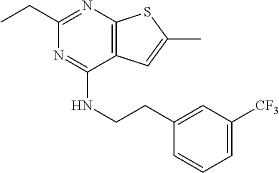 [SMILES: CCc1nc(NCCc2cccc(C(F)(F)F)c2)c2cc(C)sc2n1] | KH 4007 | Method A 2 h (140° C.) microwave | 27% | 2-ethyl-6-methyl-N- (3- (trifluoromethyl) phenethyl)thieno[2,3- d]pyrimidin-4-amine |
| 21 | 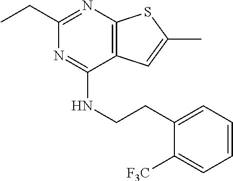 [SMILES: CCc1nc(NCCc2ccccc2C(F)(F)F)c2cc(C)sc2n1] | KH 4007 | Method A 2 h (140° C.) microwave | 52% | 2-ethyl-6-methyl-N- (2- (trifluoromethyl) phenethyl)thieno[2,3- d]pyrimidin-4-amine |
| 22 | 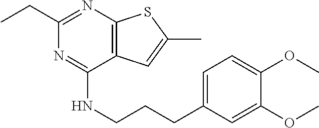 [SMILES: CCc1nc(NCCCc2ccc(OC)c(OC)c2)c2cc(C)sc2n1] | KH 4007 | Method A 2 h (140° C.) microwave | 66% | N-(3-(3,4- dimethoxyphenyl) propyl)-2-ethyl-6- methylthieno[2,3- d]pyrimidin-4-amine |
| 23 | 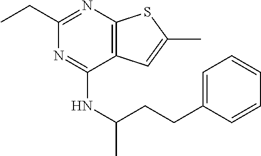 [SMILES: CCc1nc(NC(C)CCc2ccccc2)c2cc(C)sc2n1] | KH 4007 | Method A 2 h (140° C.) microwave | 12% | 2-ethyl-6-methyl-N- (4-phenylbutan-2- yl)thieno[2,3- d]pyrimidin-4-amine |
| 24 | 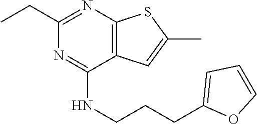 [SMILES: CCc1nc(NCCCc2ccco2)c2cc(C)sc2n1] | KH 4007 | Method A 2 h (140° C.) microwave | 75% | 2-ethyl-N-(3-(furan- 2-yl)propyl)-6- methylthieno[2,3- d]pyrimidin-4-amine |
| 25 | 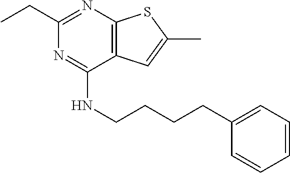 [SMILES: CCc1nc(NCCCCc2ccccc2)c2cc(C)sc2n1] | KH 4007 | Method A 2 h (140° C.) microwave | 26% | 2-ethyl-6-methyl-N- (4- phenylbutyl)thieno [2,3-d]pyrimidin-4- amine |
| 26 | 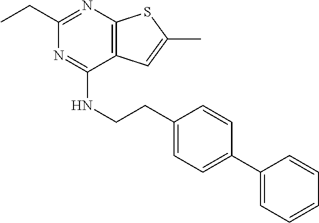 [SMILES: CCc1nc(NCCc2ccc(-c3ccccc3)cc2)c2cc(C)sc2n1] | KH 4007 | Method A 2 h (140° C.) microwave | 74% | N-(2-([1,1′- biphenyl]-4- yl)ethyl)-2-ethyl-6- methylthieno[2,3- d]pyrimidin-4-amine |
| 27 | 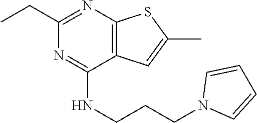 [SMILES: CCc1nc(NCCCn2cccc2)c2cc(C)sc2n1] | KH 4007 | Method A 2 h (140° C.) microwave | 80% | N-(3-(1H-pyrrol-1- yl)propyl)-2-ethyl-6- methylthieno[2,3- d]pyrimidin-4-amine |
| 28 | 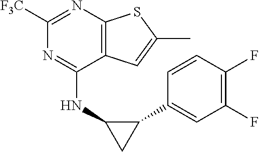 [SMILES: Cc1cc2c(N[C@@H]3C[C@H]3c3ccc(F)c(F)c3)nc(C(F)(F)F)nc2s1] | KH 6080 | Method A 2 h (160° C.) microwave | 73% | N-((1R,2S)-2-(3,4- difluorophenyl) cyclopropyl)- 6-methyl-2- (trifluoromethyl) thieno [2,3-d]pyrimidin- 4-amine |
| 29 | 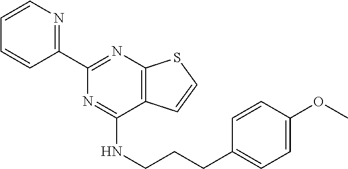 [SMILES: COc1ccc(CCCNc2nc(-c3ccccn3)nc3sccc23)cc1] | KH 4027 | Method A 2 h (140° C.) microwave | 38% | N-(3-(4- methoxyphenyl)propyl)- 2-(pyridin-2- yl)thieno[2,3- d]pyrimidin-4-amine |
| 30 | 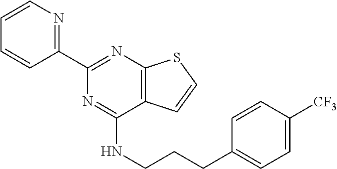 [SMILES: FC(F)(F)c1ccc(CCCNc2nc(-c3ccccn3)nc3sccc23)cc1] | KH 4027 | Method A 2 h (140° C.) microwave | 31% | 2-(pyridin-2-yl)-N- (3-(4- (trifluoromethyl) phenyl)propyl)thieno[2,3- d]pyrimidin-4- amine |
| 31 | 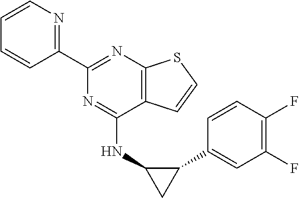 [SMILES: Fc1ccc([C@@H]2C[C@H]2Nc2nc(-c3ccccn3)nc3sccc23)cc1F] | KH 4027 | Method A 2 h (150° C.) microwave | 15% | N-((1R,2S)-2-(3,4- difluorophenyl) cyclopropyl)-2-(pyridin-2- yl)thieno[2,3- d]pyrimidin-4-amine |
| 32 | 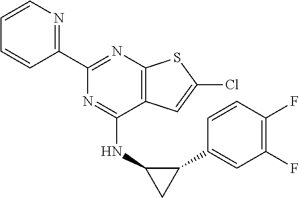 [SMILES: Fc1ccc([C@@H]2C[C@H]2Nc2nc(-c3ccccn3)nc3sc(Cl)cc23)cc1F] | KH 4037 | Method A 2 h (150° C.) microwave | 54% | 6-chloro-N-((1R,2S)- 2-(3,4- difluorophenyl) cyclopropyl)-2-(pyridin-2- yl)thieno[2,3- d]pyrimidin-4-amine |
| 33 | 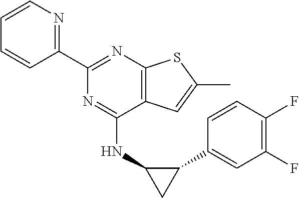 [SMILES: Cc1cc2c(N[C@@H]3C[C@H]3c3ccc(F)c(F)c3)nc(-c3ccccn3)nc2s1] | KH 4036 | Method A 2 h (150° C.) microwave | 33% | N-((1R,2S)-2-(3,4- difluorophenyl) cyclopropyl)-6-methyl-2- (pyridin-2- yl)thieno[2,3- d]pyrimidin-4-amine |
| 34 | 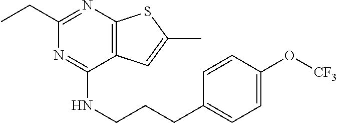 [SMILES: CCc1nc(NCCCc2ccc(OC(F)(F)F)cc2)c2cc(C)sc2n1] | KH 4007 | Method A 2 h (140° C.) microwave | 52% | 2-ethyl-6-methyl-N- (3-(4- (trifluoromethoxy) phenyl)propyl)thieno[2, 3-d]pyrimidin-4- amine |
| 35 | 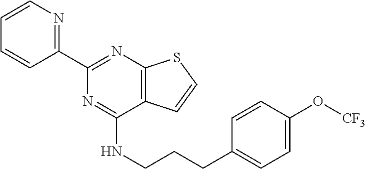 [SMILES: FC(F)(F)Oc1ccc(CCCNc2nc(-c3ccccn3)nc3sccc23)cc1] | KH 4027 | Method A 2 h (140° C.) microwave | 33% | 2-(pyridin-2-yl)-N- (3-(4- (trifluoromethoxy) phenyl)propyl)thieno[2, 3-d]pyrimidin-4- amine |
| 36 | 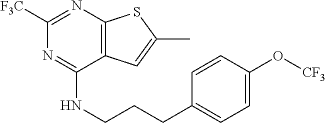 [SMILES: Cc1cc2c(NCCCc3ccc(OC(F)(F)F)cc3)nc(C(F)(F)F)nc2s1] | KH 6080 | Method A 2 h (140° C.) microwave | 78% | 6-methyl-N-(3-(4- (trifluoromethoxy) phenyl)propyl)-2- (trifluoromethyl)thieno [2,3-d]pyrimidin- 4-amine |
| 37 | 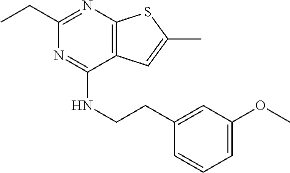 [SMILES: CCc1nc(NCCc2cccc(OC)c2)c2cc(C)sc2n1] | KH 4007 | Method A 2 h (140° C.) microwave | 31% | 2-ethyl-N-(3- methoxyphenethyl)- 6-methylthieno[2,3- d]pyrimidin-4-amine |
| 38 | 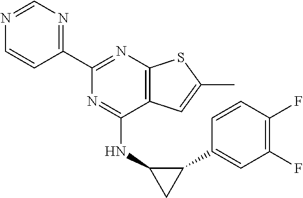 [SMILES: Cc1cc2c(N[C@@H]3C[C@H]3c3ccc(F)c(F)c3)nc(-c3ccncn3)nc2s1] | KH 4057 | Method A 2 h (150° C.) microwave | 43% | N-((1R,2S)-2-(3,4- difluorophenyl)cyclopropyl)- 6-methyl-2- (pyrimidin-4- yl)thieno[2,3- d]pyrimidin-4-amine |
| 39 | 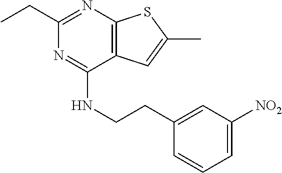 [SMILES: CCc1nc(NCCc2cccc([N+](=O)[O-])c2)c2cc(C)sc2n1] | KH 4007 | Method A 2 h (140° C.) microwave | 44% | 2-ethyl-6-methyl-N- (3- nitrophenethyl)thieno [2,3-d]pyrimidin-4- amine |
| 40 | 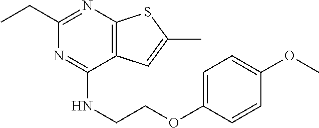 [SMILES: CCc1nc(NCCOc2ccc(OC)cc2)c2cc(C)sc2n1] | KH 4007 | Method A 2 h (140° C.) microwave | 62% | 2-ethyl-N-(2-(4- methoxyphenoxy) ethyl)-6- methylthieno[2,3- d]pyrimidin-4-amine |
| 41 | 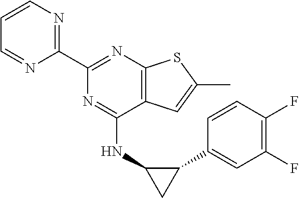 [SMILES: Cc1cc2c(N[C@@H]3C[C@H]3c3ccc(F)c(F)c3)nc(-c3ncccn3)nc2s1] | KH 4069 | Method A 2 h (150° C.) microwave | 29% | N-((1R,2S)-2-(3,4- difluorophenyl) cyclopropyl)-6-methyl-2- (pyrimidin-2- yl)thieno[2,3- d]pyrimidin-4-amine |
| 42 | 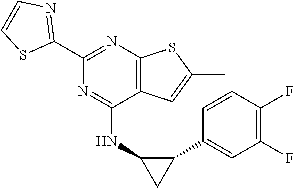 [SMILES: Cc1cc2c(N[C@@H]3C[C@H]3c3ccc(F)c(F)c3)nc(-c3nccs3)nc2s1] | KH 4070 | Method A 2 h (150° C.) microwave | 27% | N-((1R,2S)-2-(3,4- difluorophenyl) cyclopropyl)-6-methyl-2- (thiazol-2- yl)thieno[2,3- d]pyrimidin-4-amine |
| 43 | 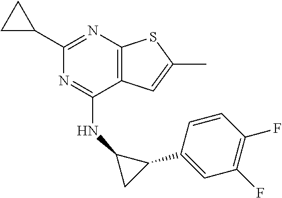 [SMILES: Cc1cc2c(N[C@@H]3C[C@H]3c3ccc(F)c(F)c3)nc(C3CC3)nc2s1] | KH 4082 | Method A 2 h (150° C.) microwave | 12% | 2-cyclopropyl-N- ((1R,2S)-2-(3,4- difluorophenyl) cyclopropyl)-6- methylthieno[2,3- d]pyrimidin-4-amine |
| 44 | 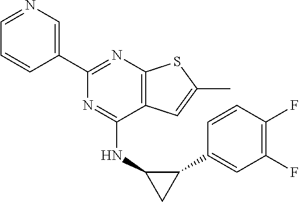 [SMILES: Cc1cc2c(N[C@@H]3C[C@H]3c3ccc(F)c(F)c3)nc(-c3cccnc3)nc2s1] | KH 4083 | Method A 2 h (150° C.) microwave | 18% | N-((1R,2S)-2-(3,4- difluorophenyl) cyclopropyl)-6-methyl-2- (pyridin-3- yl)thieno[2,3- d]pyrimidin-4-amine |
| 45 | 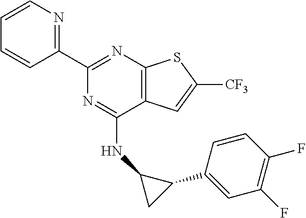 [SMILES: Fc1ccc([C@@H]2C[C@H]2Nc2nc(-c3ccccn3)nc3sc(C(F)(F)F)cc23)cc1F] | KH 4084 | Method A 2 h (150° C.) microwave | 59% | N-((1R,2S)-2-(3,4- difluorophenyl) cyclopropyl)-2-(pyridin-2- yl)-6- (trifluoromethyl)thieno [2,3-d]pyrimidin- 4-amine |
| 46 | 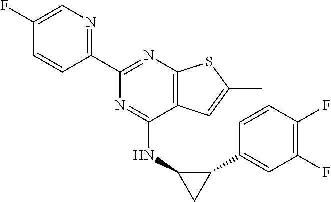 [SMILES: Cc1cc2c(N[C@@H]3C[C@H]3c3ccc(F)c(F)c3)nc(-c3ccc(F)cn3)nc2s1] | KH 4085 | Method A 2 h (150° C.) microwave | 21% | N-((1R,2S)-2-(3,4- difluorophenyl) cyclopropyl)-2-(5- fluoropyridin-2-yl)- 6-methylthieno[2,3- d]pyrimidin-4-amine |
| 47 | 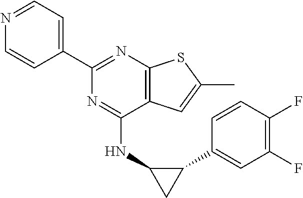 [SMILES: Cc1cc2c(N[C@@H]3C[C@H]3c3ccc(F)c(F)c3)nc(-c3ccncc3)nc2s1] | KH 4086 | Method A 2 h (140° C.) microwave | 11% | N-((1R,2S)-2-(3,4- difluorophenyl) cyclopropyl)-6-methyl-2- (pyridin-4- yl)thieno[2,3- d]pyrimidin-4-amine |
| 48 | 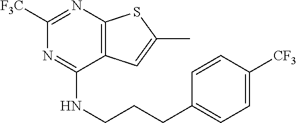 [SMILES: Cc1cc2c(NCCCc3ccc(C(F)(F)F)cc3)nc(C(F)(F)F)nc2s1] | KH 6080 | Method A 2 h (150° C.) microwave | 77% | 6-methyl-2- (trifluoromethyl)-N- (3-(4- (trifluoromethyl) phenyl)propyl)thieno[2,3-d] pyrimidin-4- amine |
| 49 | 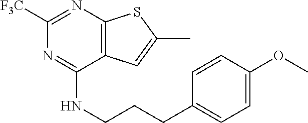 [SMILES: COc1ccc(CCCNc2nc(C(F)(F)F)nc3sc(C)cc23)cc1] | KH 6080 | Method A 2 h (150° C.) microwave | 85% | N-(3-(4- methoxyphenyl) propyl)-6-methyl-2- (trifluoromethyl)thieno [2,3-d]pyrimidin- 4-amine |
| 50 | 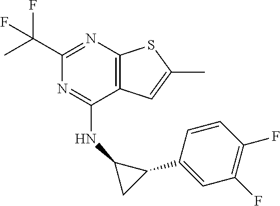 [SMILES: Cc1cc2c(N[C@@H]3C[C@H]3c3ccc(F)c(F)c3)nc(C(C)(F)F)nc2s1] | KH 4100 | Method A 2 h (150° C.) microwave | 59% | 2-(1, 1-difluoroethyl)- N-((1R,2S)-2-(3,4- difluorophenyl) cyclopropyl)-6- methylthieno[2,3- d]pyrimidin-4-amine |
| 51 | 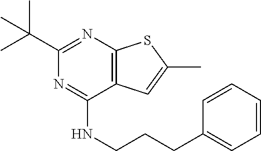 [SMILES: Cc1cc2c(NCCCc3ccccc3)nc(C(C)(C)C)nc2s1] | KH 4099 | Method A 2 h (150° C.) microwave | 61% | 2-(tert-butyl)-6- methyl-N-(3- phenylpropyl)thieno [2,3-d]pyrimidin-4- amine |
| 52 | 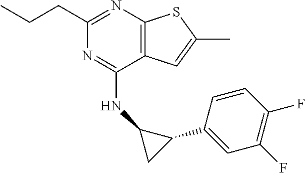 [SMILES: CCCc1nc(N[C@@H]2C[C@H]2c2ccc(F)c(F)c2)c2cc(C)sc2n1] | KH 5001 | Method A 2 h (150° C.) microwave | 10% | N-((1R,2S)-2-(3,4- difluorophenyl) cyclopropyl)-6-methyl-2- propylthieno[2,3- d]pyrimidin-4-amine |
| 53 | 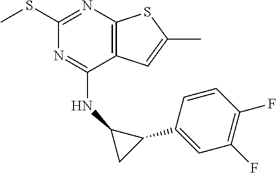 [SMILES: CSc1nc(N[C@@H]2C[C@H]2c2ccc(F)c(F)c2)c2cc(C)sc2n1] | KH 5002 | Method A 1 h (140° C.) microwave | 24% | N-((1R,2S)-2-(3,4- difluorophenyl) cyclopropyl)-6-methyl-2- (methylthio)thieno[2, 3-d]pyrimidin-4- amine |
| 54 | 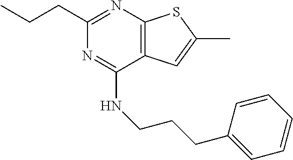 [SMILES: CCCc1nc(NCCCc2ccccc2)c2cc(C)sc2n1] | KH 5001 | Method A 1 h (150° C.) microwave | 55% | 6-methyl-N-(3- phenylpropyl)-2- propylthieno[2,3- d]pyrimidin-4-amine |
| 55 | 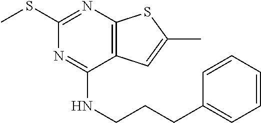 [SMILES: CSc1nc(NCCCc2ccccc2)c2cc(C)sc2n1] | KH 5002 | Method A 1 h (150° C.) microwave | 72% | 6-methyl-2- (methylthio)-N-(3- phenylpropyl)thieno [2,3-d]pyrimidin-4- amine |
| 56 | 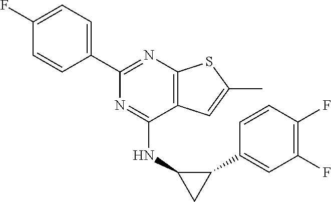 [SMILES: Cc1cc2c(N[C@@H]3C[C@H]3c3ccc(F)c(F)c3)nc(-c3ccc(F)cc3)nc2s1] | KH 5015 | Method A 2 h (120° C.) microwave | 15% | N-((1R,2S)-2-(3,4- difluorophenyl) cyclopropyl)-2-(4- fluorophenyl)-6- methylthieno[2,3- d]pyrimidin-4-amine |
| 57 | 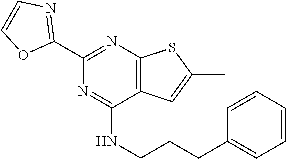 [SMILES: Cc1cc2c(NCCCc3ccccc3)nc(-c3ncco3)nc2s1] | KH 5016 | Method A 1 h (140° C.) microwave | 48% | 6-methyl-2-(oxazol- 2-yl)-N-(3- phenylpropyl)thieno [2,3-d]pyrimidin-4- amine |
| 58 | 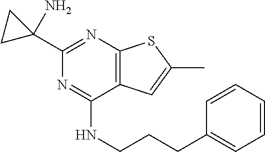 [SMILES: Cc1cc2c(NCCCc3ccccc3)nc(C3(N)CC3)nc2s1] | KH 5017 | Method A 1 h (140° C.) microwave | 50% | 2-(1- aminocyclopropyl)- 6-methyl-N-(3- phenylpropyl)thieno [2,3-d]pyrimidin-4- amine |
| 59 | 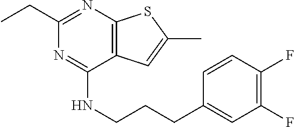 [SMILES: CCc1nc(NCCCc2ccc(F)c(F)c2)c2cc(C)sc2n1] | KH 4007 | Method A 1 h (140° C.) microwave | 33% | N-(3-(3,4- difluorophenyl) propyl)-2-ethyl-6- methylthieno[2,3- d]pyrimidin-4-amine |
| 60 | 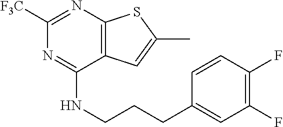 [SMILES: Cc1cc2c(NCCCc3ccc(F)c(F)c3)nc(C(F)(F)F)nc2s1] | KH 6080 | Method A 1 h (140° C.) microwave | 74% | N-(3-(3,4- difluorophenyl)propyl)- 6-methyl-2- (trifluoromethyl)thieno [2,3-d]pyrimidin- 4-amine |
| 61 | 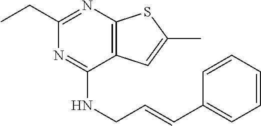 [SMILES: CCc1nc(NC/C=C/c2ccccc2)c2cc(C)sc2n1] | KH 4007 | Method A 1 h (140° C.) microwave | 24% | N-cinnamyl-2-ethyl- 6-methylthieno[2,3- d]pyrimidin-4-amine |
| 62 | 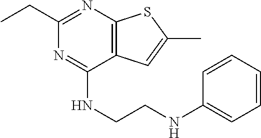 [SMILES: CCc1nc(NCCNc2ccccc2)c2cc(C)sc2n1] | KH 4007 | Method A 1 h (140° C.) microwave | 55% | N1-(2-ethyl-6- methylthieno[2,3- d]pyrimidin-4-yl)- N2-phenylethane- 1,2-diamine |
| 63 | 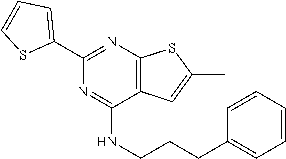 [SMILES: Cc1cc2c(NCCCc3ccccc3)nc(-c3cccs3)nc2s1] | KH 4076 | Method A 1 h (140° C.) microwave | 62% | 6-methyl-N-(3- phenylpropyl)-2- (thiophen-2- yl)thieno[2,3- d]pyrimidin-4-amine |
| 64 | 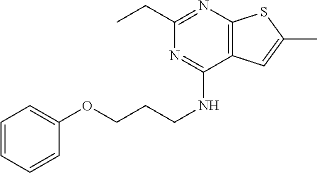 [SMILES: CCc1nc(NCCCOc2ccccc2)c2cc(C)sc2n1] | KH 4007 | Method A 1 h (140° C.) microwave | 62% | 2-ethyl-6-methyl-N- (3- phenoxypropyl)thieno [2,3-d]pyrimidin-4- amine |
| 65 | 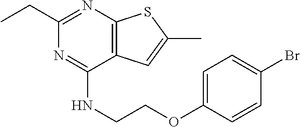 [SMILES: CCc1nc(NCCOc2ccc(Br)cc2)c2cc(C)sc2n1] | KH 4007 | Method A 1 h (140° C.) microwave | 41% | N-(2-(4- bromophenoxy)ethyl)- 2-ethyl-6- methylthieno[2,3- d]pyrimidin-4-amine |
| 66 | 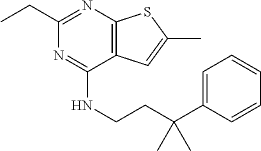 [SMILES: CCc1nc(NCCC(C)(C)c2ccccc2)c2cc(C)sc2n1] | KH 4007 | Method A 1 h (140° C.) microwave | 60% | 2-ethyl-6-methyl-N- (3-methyl-3- phenylbutyl)thieno [2,3-d]pyrimidin-4- amine |
| 67 | 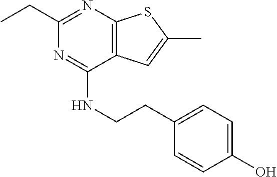 [SMILES: CCc1nc(NCCc2ccc(O)cc2)c2cc(C)sc2n1] | KH 4007 | Method A 1 h (140° C.) microwave | 42% | 4-(2-((2-ethyl-6- methylthieno[2,3- d]pyrimidin-4- yl)amino)ethyl)phenol |
| 68 | 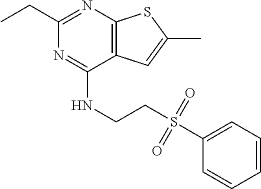 [SMILES: CCc1nc(NCCS(=O)(=O)c2ccccc2)c2cc(C)sc2n1] | KH 4007 | Method A 1 h (140° C.) microwave | 19% | 2-ethyl-6-methyl-N-(2- (phenylsulfonyl)ethyl) thieno[2,3- d]pyrimidin-4-amine |
| 69 | 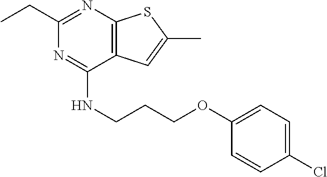 [SMILES: CCc1nc(NCCCOc2ccc(Cl)cc2)c2cc(C)sc2n1] | KH 4007 | Method A 1 h (140° C.) microwave | 36% | N-(3-(4- chlorophenoxy) propyl)-2-ethyl-6- methylthieno[2,3- d]pyrimidin-4-amine |
| 70 | 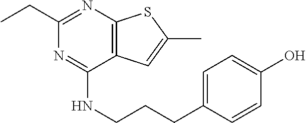 [SMILES: CCc1nc(NCCCc2ccc(O)cc2)c2cc(C)sc2n1] | KH 4007 | Method A 1 h (140° C.) microwave | 33% | 4-(3-((2-ethyl-6- methylthieno[2,3- d]pyrimidin-4- yl)amino)propyl) phenol |
| 71 | 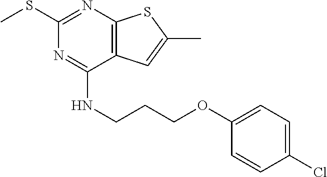 [SMILES: CSc1nc(NCCCOc2ccc(Cl)cc2)c2cc(C)sc2n1] | KH 5002 | Method A 1 h (140° C.) microwave | 47% | N-(3-(4- chlorophenoxy) propyl)-6-methyl-2- (methylthio)thieno[2, 3-d]pyrimidin-4- amine |
| 72 | 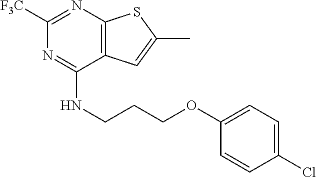 [SMILES: Cc1cc2c(NCCCOc3ccc(Cl)cc3)nc(C(F)(F)F)nc2s1] | KH 6080 | Method A 1 h (140° C.) microwave | 53% | N-(3-(4- chlorophenoxy) propyl)-6-methyl-2- (trifluoromethyl)thieno [2,3-d]pyrimidin- 4-amine |
| 73 | 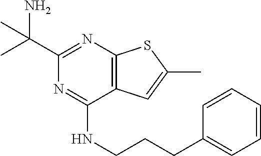 [SMILES: Cc1cc2c(NCCCc3ccccc3)nc(C(C)(C)N)nc2s1] | KH 5044 | Method A 1 h (140° C.) microwave | 39% | 2-(2-aminopropan-2- yl)-6-methyl-N-(3- phenylpropyl)thieno [2,3-d]pyrimidin-4- amine |
| 74 | 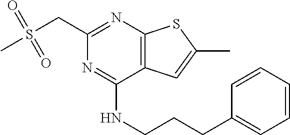 [SMILES: Cc1cc2c(NCCCc3ccccc3)nc(CS(C)(=O)=O)nc2s1] | KH 5045 | Method A 1 h (140° C.) microwave | 46% | 6-methyl-2- ((methylsulfonyl) methyl)-N-(3- phenylpropyl)thieno [2,3-d]pyrimidin-4- amine |
| 75 | 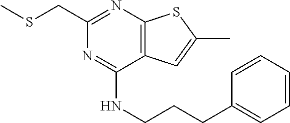 [SMILES: CSCc1nc(NCCCc2ccccc2)c2cc(C)sc2n1] | KH 5046 | Method A 1 h (140° C.) microwave | 55% | 6-methyl-2- ((methylthio)methyl)- N-(3- phenylpropyl)thieno [2,3-d]pyrimidin-4- amine |
| 76 | 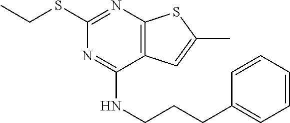 [SMILES: CCSc1nc(NCCCc2ccccc2)c2cc(C)sc2n1] | KH 5047 | Method A 1 h (140° C.) microwave | 33% | 2-(ethylthio)-6- methyl-N-(3- phenylpropyl)thieno [2,3-d]pyrimidin-4- amine |
| 77 | 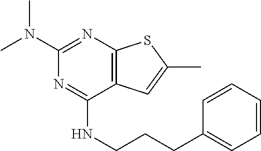 [SMILES: Cc1cc2c(NCCCc3ccccc3)nc(N(C)C)nc2s1] | KH 5048 | Method A 1 h (140° C.) microwave | 15% | N2,N2,6-trimethyl- N4-(3- phenylpropyl)thieno [2,3-d]pyrimidine- 2,4-diamine |
| 78 | 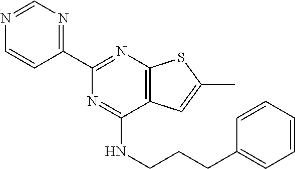 [SMILES: Cc1cc2c(NCCCc3ccccc3)nc(-c3ccncn3)nc2s1] | KH 4057 | Method A 1 h (100° C.) microwave | 53% | 6-methyl-N-(3- phenylpropyl)-2- (pyrimidin-4- yl)thieno[2,3- d]pyrimidin-4-amine |
| 79 | 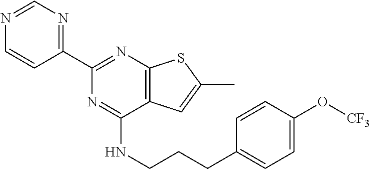 [SMILES: Cc1cc2c(NCCCc3ccc(OC(F)(F)F)cc3)nc(-c3ccncn3)nc2s1] | KH 4057 | Method A 1 h (100° C.) microwave | 28% | 6-methyl-2- (pyrimidin-4-yl)-N- (3-(4- (trifluoromethoxy) phenyl)propyl)thieno [2,3-d]pyrimidin-4- amine |
| 80 | 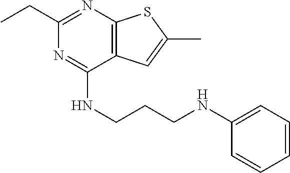 [SMILES: CCc1nc(NCCCNc2ccccc2)c2cc(C)sc2n1] | KH 4007 | Method A 1 h (100° C.) microwave | 28% | N1-(2-ethyl-6- methylthieno[2,3- d]pyrimidin-4-yl)- N3-phenylpropane- 1,3-diamine |
| 81 | 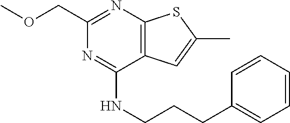 [SMILES: COCc1nc(NCCCc2ccccc2)c2cc(C)sc2n1] | KH 5062 | Method A 1 h (140° C.) microwave | 70% | 2-(methoxymethyl)- 6-methyl-N-(3- phenylpropyl)thieno [2,3-d]pyrimidin-4- amine |
| 82 | 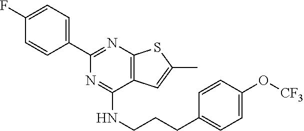 [SMILES: Cc1cc2c(NCCCc3ccc(OC(F)(F)F)cc3)nc(-c3ccc(F)cc3)nc2s1] | KH 5015 | Method A 1 h (140° C.) microwave | 55% | 2-(4-fluorophenyl)- 6-methyl-N-(3-(4- (trifluoromethoxy) phenyl)propyl)thieno[2, 3-d]pyrimidin-4- amine |
| 83 | 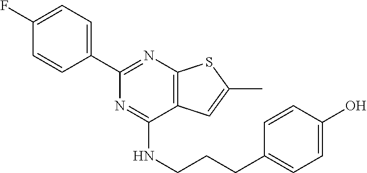 [SMILES: Cc1cc2c(NCCCc3ccc(O)cc3)nc(-c3ccc(F)cc3)nc2s1] | KH 5015 | Method A 1 h (140° C.) microwave | 17% | 4-(3-((2-(4- fluorophenyl)-6- methylthieno[2,3- d]pyrimidin-4- yl)amino)propyl) phenol |
| 84 | 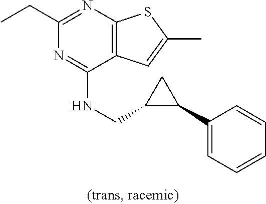 [SMILES: CCc1nc(NC[C@@H]2C[C@H]2c2ccccc2)c2cc(C)sc2n1] | KH 4007 | Method A 2 h (120° C.) microwave | 24% | 2-ethyl-6-methyl-N- (((1S,2S)-2- phenylcyclopropyl) methyl)thieno[2,3- d]pyrimidin-4-amine |
| 85 | 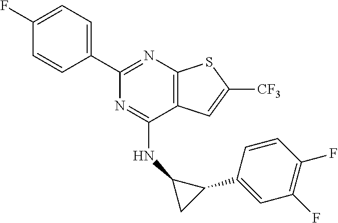 [SMILES: Fc1ccc(-c2nc(N[C@@H]3C[C@H]3c3ccc(F)c(F)c3)c3cc(C(F)(F)F)sc3n2)cc1] | KH 5094 | Method A 2 h (120° C.) microwave | 16% | N-((1R,2S)-2-(3,4- difluorophenyl) cyclopropyl)-2- (4- fluorophenyl)-6- (trifluoromethyl)thieno [2,3-d]pyrimidin- 4-amine |
| 86 | 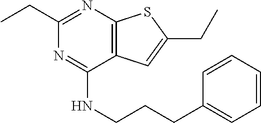 [SMILES: CCc1nc(NCCCc2ccccc2)c2cc(CC)sc2n1] | KH 5091 | Method A 1 h (140° C.) microwave | 57% | 2,6-diethyl-N-(3- phenylpropyl)thieno [2,3-d]pyrimidin-4- amine |
| 87 | 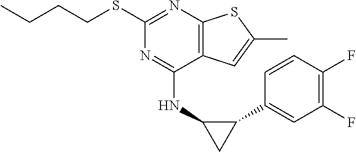 [SMILES: CCCCSc1nc(N[C@@H]2C[C@H]2c2ccc(F)c(F)c2)c2cc(C)sc2n1] | KH 5090 | Method A 2 h (120° C.) microwave | 48% | 2-(butylthio)-N- ((1R,2S)-2-(3,4- difluorophenyl) cyclopropyl)-6- methylthieno[2,3- d]pyrimidin-4-amine |
| 88 | 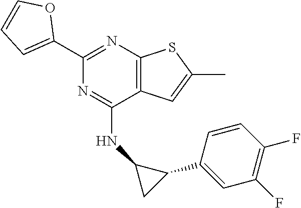 [SMILES: Cc1cc2c(N[C@@H]3C[C@H]3c3ccc(F)c(F)c3)nc(-c3ccco3)nc2s1] | KH 5088 | Method A 2 h (120° C.) microwave | 8% | N-((1R,2S)-2-(3,4- difluorophenyl) cyclopropyl)-2- (furan-2-yl)-6- methylthieno[2,3- d]pyrimidin-4-amine |
| 89 | 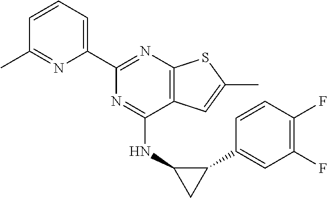 [SMILES: Cc1cccc(-c2nc(N[C@@H]3C[C@H]3c3ccc(F)c(F)c3)c3cc(C)sc3n2)n1] | KH 5096 | Method A 2 h (120° C.) microwave | 19% | N-((1R,2S)-2-(3,4- difluorophenyl) cyclopropyl)-6-methyl- 2-(6-methylpyridin-2- yl)thieno[2,3- d]pyrimidin-4-amine |
| 90 | 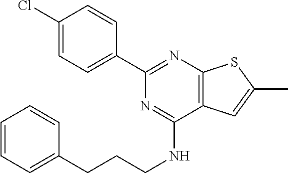 [SMILES: Cc1cc2c(NCCCc3ccccc3)nc(-c3ccc(Cl)cc3)nc2s1] | KH 5093 | Method A 1 h (140° C.) microwave | 66% | 2-(4-chlorophenyl)- 6-methyl-N-(3- phenylpropyl)thieno [2,3-d]pyrimidin-4- amine |
| 91 | 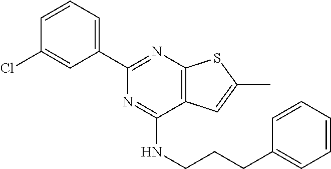 [SMILES: Cc1cc2c(NCCCc3ccccc3)nc(-c3cccc(Cl)c3)nc2s1] | KH 5089 | Method A 1 h (140° C.) microwave | 55% | 2-(3-chlorophenyl)- 6-methyl-N-(3- phenylpropyl)thieno [2,3-d]pyrimidin-4- amine |
| 92 | 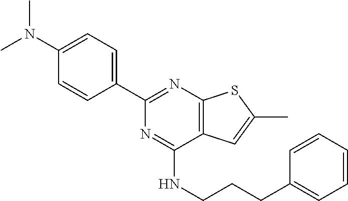 [SMILES: Cc1cc2c(NCCCc3ccccc3)nc(-c3ccc(N(C)C)cc3)nc2s1] | KH 5095 | Method A 1 h (140° C.) microwave | 28% | 2-(4- (dimethylamino) phenyl)-6-methyl-N-(3- phenylpropyl)thieno [2,3-d]pyrimidin-4- amine |
| 93 | 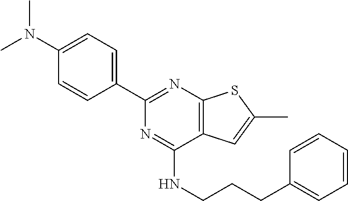 [SMILES: Cc1cc2c(NCCCc3ccccc3)nc(-c3ccc(N(C)C)cc3)nc2s1] | KH 6017 | Method A 1 h (140° C.) microwave | 16% | N2-cyclopropyl- N2,6-dimethyl-N4- (3- phenylpropyl)thieno [2,3-d]pyrimidine- 2,4-diamine |
| 94 | 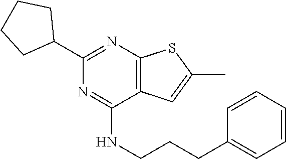 [SMILES: Cc1cc2c(NCCCc3ccccc3)nc(C3CCCC3)nc2s1] | KH 6019 | Method A 1 h (140° C.) microwave | 61% | 2-cyclopentyl-6- methyl-N-(3- phenylpropyl)thieno [2,3-d]pyrimidin-4- amine |
| 95 | 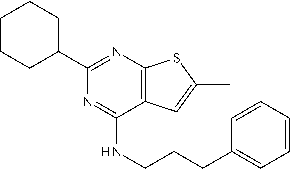 [SMILES: Cc1cc2c(NCCCc3ccccc3)nc(C3CCCCC3)nc2s1] | KH 6020 | Method A 1 h (140° C.) microwave | 30% | 2-cyclohexyl-6- methyl-N-(3- \|phenylpropyl)thieno [2,3-d]pyrimidin-4- amine |
| 96 | 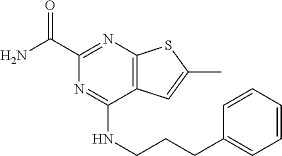 [SMILES: Cc1cc2c(NCCCc3ccccc3)nc(C(N)=O)nc2s1] | KH 6094 | Method A 1 h (140° C.) microwave | 64% | 6-methyl-4-((3- phenylpropyl)amino) thieno[2,3- d]pyrimidine-2- carboxamide |
| 97 | 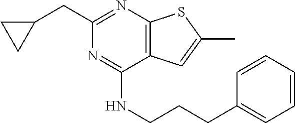 [SMILES: Cc1cc2c(NCCCc3ccccc3)nc(CC3CC3)nc2s1] | KH 6018 | Method A 1 h (140° C.) microwave | 77% | 2- (cyclopropyLMethyl)- 6-methyl-N-(3. phenylpropyl)thieno [2,3-d]pyrimidin-4- amine |
| 98 | 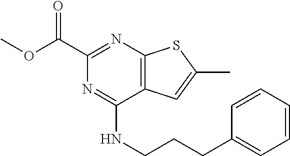 [SMILES: COC(=O)c1nc(NCCCc2ccccc2)c2cc(C)sc2n1] | KH 6029 | Method A 1 h (140° C.) microwave | 50% | methyl 6-methyl-4- ((3- phenylpropyl)amino) thieno[2,3- d]pyrimidine-2- carboxylate |
| 99 | 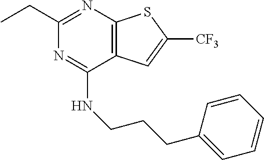 [SMILES: CCc1nc(NCCCc2ccccc2)c2cc(C(F)(F)F)sc2n1] | KH 6061 | Method A 1 h (140° C.) microwave | 29% | 2-ethyl-N-(3- phenylpropyl)-6- (trifluoromethyl)thieno [2,3-d]pyrimidin- 4-amine |
| 100 | 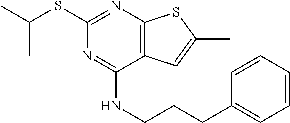 [SMILES: Cc1cc2c(NCCCc3ccccc3)nc(SC(C)C)nc2s1] | KH 6065 | Method A 1 h (140° C.) microwave | 47% | 2-(isopropylthio)-6- methyl-N-(3- phenylpropyl)thieno [2,3-d]pyrimidin-4- amine |
| 101 | 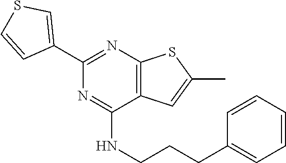 [SMILES: Cc1cc2c(NCCCc3ccccc3)nc(-c3ccsc3)nc2s1] | KH 6066 | Method A 1 h (140° C.) microwave | 63% | 6-methyl-N-(3- phenylpropyl)-2- (thiophen-3- yl)thieno[2,3- d]pyrimidin-4-amine |
| 102 | 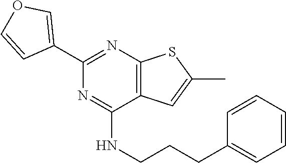 [SMILES: Cc1cc2c(NCCCc3ccccc3)nc(-c3ccoc3)nc2s1] | KH 6067 | Method A 1 h (140° C.) microwave | 55% | 2-(furan-3-yl)-6- methyl-N-(3- phenylpropyl)thieno [2,3-d]pyrimidin-4- amine |
| 103 | 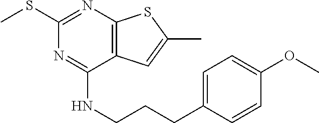 | KH 5002 | Method A 2 d (110° C.) | 65% | N-(3-(4- methoxyphenyl) propyl)-6-methyl-2- (methylthio)thieno[2, 3-d]pyrimidin-4- amine |
| 104 | 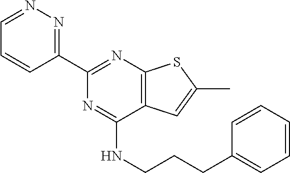 [SMILES: Cc1cc2c(NCCCc3ccccc3)nc(-c3cccnn3)nc2s1] | KH 6089 | Method A 1 h (140° C.) microwave | 41% | 6-methyl-N-(3- phenylpropyl)-2- (pyridazin-3- yl)thieno[2,3- d]pyrimidin-4-amine |
| 105 | 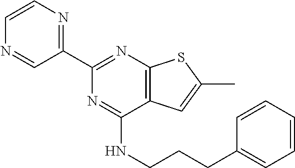 [SMILES: Cc1cc2c(NCCCc3ccccc3)nc(-c3cnccn3)nc2s1] | KH 6090 | Method A 1 h (140° C.) microwave | 75% | 6-methyl-N-(3- phenylpropyl)-2- (pyrazin-2- yl)thieno[2,3- d]pyrimidin-4-amine |
| 106 | 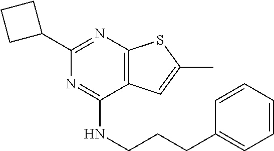 [SMILES: Cc1cc2c(NCCCc3ccccc3)nc(C3CCC3)nc2s1] | KH 6091 | Method A 1 h (140° C.) microwave | 76% | 2-cyclobutyl-6- methyl-N-(3- phenylpropyl)thieno [2,3-d]pyrimidin-4- amine |
| 107 | 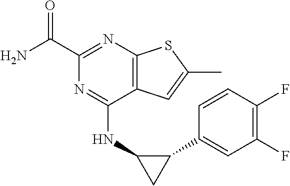 [SMILES: Cc1cc2c(N[C@@H]3C[C@H]3c3ccc(F)c(F)c3)nc(C(N)=O)nc2s1] | KH 6094 | Method A 1 h (140° C.) microwave | 58% | 4-(((1R,2S)-2-(3,4- difluorophenyl) cyclopropyl)amino)-6- methylthieno[2,3- d]pyrimidine-2- carboxamide |
| 108 | 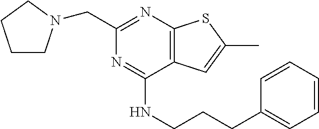 [SMILES: Cc1cc2c(NCCCc3ccccc3)nc(CN3CCCC3)nc2s1] | KH 6092 | Method A 1 h (140° C.) microwave | 17% | 6-methyl-N-(3- phenylpropyl)-2- (pyrrolidin-1-yLM ethyl)thieno[2,3- d]pyrimidin-4-amine |
| 109 | 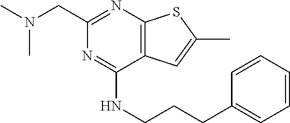 [SMILES: Cc1cc2c(NCCCc3ccccc3)nc(CN(C)C)nc2s1] | KH 6093 | Method A 1 h (140° C.) microwave | 16% | 2- ((dimethylamino) methyl)-6-methyl-N- (3-phenylpropyl)thieno [2,3-d]pyrimidin-4- amine |
| 110 | 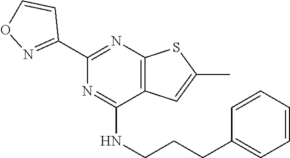 [SMILES: Cc1cc2c(NCCCc3ccccc3)nc(-c3ccon3)nc2s1] | KH 7018 | Method A 1 h (140° C.) microwave | 69% | 2-(isoxazol-3-yl)-6- methyl-N-(3- phenylpropyl)thieno [2,3-d]pyrimidin-4- amine |
| 111 | 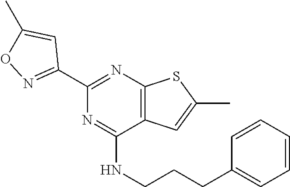 [SMILES: Cc1cc(-c2nc(NCCCc3ccccc3)c3cc(C)sc3n2)no1] | KH 7019 | Method A 1 h (140° C.) microwave | 53% | 6-methyl-2-(5- methylisoxazol-3- yl)-N-(3- phenylpropyl)thieno [2,3-d]pyrimidin-4- amin |
| 112 | 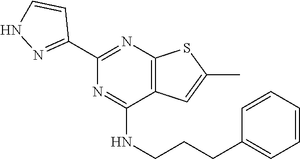 [SMILES: Cc1cc2c(NCCCc3ccccc3)nc(-c3cc[nH]n3)nc2s1] | KH 7020 | Method A 1 h (140° C.) microwave | 41% | 6-methyl-N-(3- phenylpropyl)-2- (1H-pyrazol-3- yl)thieno[2,3- d]pyrimidin-4-amine |
| 113 | 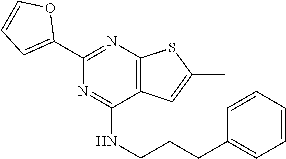 [SMILES: Cc1cc2c(NCCCc3ccccc3)nc(-c3ccco3)nc2s1] | KH 5088 | Method A 1 h (140° C.) microwave | 43% | 2-(furan-2-yl)-6- methyl-N-(3- phenylpropyl)thieno 2,3-d]pyrimidin-4- amine |
| 114 | 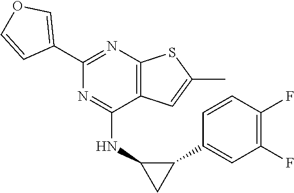 [SMILES: Cc1cc2c(N[C@@H]3C[C@H]3c3ccc(F)c(F)c3)nc(-c3ccoc3)nc2s1] | KH 6067 | Method A 1 h (140° C.) microwave | 22% | N-((1R,2S)-2-(3,4- difluorophenyl) cyclopropyl)- 2-(furan-3- yl)-6- methylthieno[2,3- d]pyrimidin-4-amine |
| 115 | 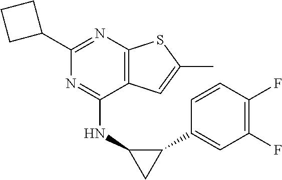 [SMILES: Cc1cc2c(N[C@@H]3C[C@H]3c3ccc(F)c(F)c3)nc(C3CCC3)nc2s1] | KH 6091 | Method A 1 h (140° C.) microwave | 13% | 2-cyclobutyl-N- ((1R,2S)-2-(3,4- difluorophenyl) cyclopropyl)-6- methylthieno[2,3- d]pyrimidin-4-amine |
| 116 | 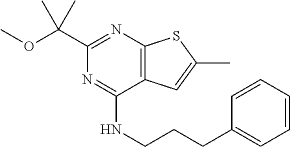 [SMILES: COC(C)(C)c1nc(NCCCc2ccccc2)c2cc(C)sc2n1] | KH 7033 | Method A 1 h (140° C.) microwave | 63% | 2-(2-methoxypropan- 2-yl)-6-methyl-N-(3- phenylpropyl)thieno [2,3-d]pyrimidin-4- amine |
| 117 | 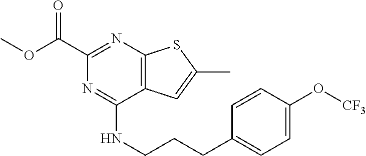 [SMILES: COC(=O)c1nc(NCCCc2ccc(OC(F)(F)F)cc2)c2cc(C)sc2n1] | KH 6029 | Method A 1 h (140° C.) microwave | 52% | methyl 6-methyl-4- ((3-(4- (trifluoromethoxy) phenyl)propyl)amino) thieno[2,3- d]pyrimidine-2- carboxylate |
| 118 | 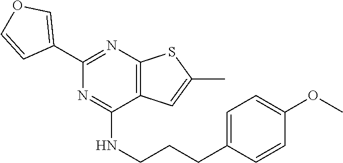 [SMILES: COc1ccc(CCCNc2nc(-c3ccoc3)nc3sc(C)cc23)cc1] | KH 6067 | Method A 1 h (150° C.) microwave | 16% | 2-(furan-3- yl)-N-(3- (4-methoxyphenyl) propyl)-6- methylthieno[2,3- d]pyrimidin-4-amine |
| 119 | 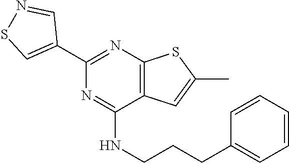 [SMILES: Cc1cc2c(NCCCc3ccccc3)nc(-c3cnsc3)nc2s1] | KH 7048 | Method A 1 h (140° C.) microwave | 73% | 2-(isothiazol-4-yl)-6- methyl-N-(3- phenylpropyl)thieno [2,3-d]pyrimidin-4- amine |
| 120 | 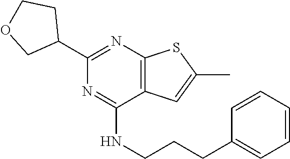 [SMILES: Cc1cc2c(NCCCc3ccccc3)nc(C3CCOC3)nc2s1] | KH 7049 | Method A 1 h (140° C.) microwave | 36% | 6-methyl-N-(3- phenylpropyl)-2- (tetrahydrofuran-3- yl)thieno[2,3- d]pyrimidin-4-amine |
| 121 | 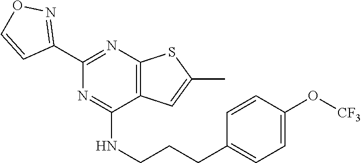 [SMILES: Cc1cc2c(NCCCc3ccc(OC(F)(F)F)cc3)nc(-c3ccon3)nc2s1] | KH 7018 | Method A 1 h (140° C.) microwave | 78% | 2-(isoxazol-3-yl)-6- methyl-N-(3-(4- (trifluoromethoxy) phenyl)propyl)thieno[2, 3-d]pyrimidin-4- amine |
| 122 | 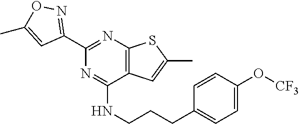 [SMILES: Cc1cc(-c2nc(NCCCc3ccc(OC(F)(F)F)cc3)c3cc(C)sc3n2)no1] | KH 7019 | Method A 1 h (140° C.) microwave | 51% | 6-methyl-2-(5- methylisoxazol-3- yl)-N-(3-(4- (trifluoromethoxy) phenyl)propyl)thieno [2,3-d]pyrimidin-4- amine |
| 123 | 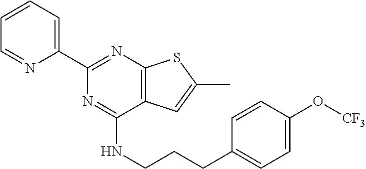 [SMILES: Cc1cc2c(NCCCc3ccc(OC(F)(F)F)cc3)nc(-c3ccccn3)nc2s1] | KH 4036 | Method A 1 h (140° C.) microwave | 57% | 6-methyl-2-(pyridin- 2-yl)-N-(3-(4- (trifluoromethoxy) phenyl)propyl)thieno [2,3-d]pyrimidin-4- amine |
| 124 | 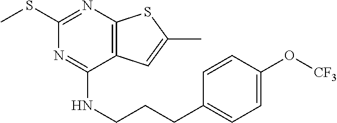 [SMILES: CSc1nc(NCCCc2ccc(OC(F)(F)F)cc2)c2cc(C)sc2n1] | KH 5002 | Method A 1 h (140° C.) microwave | 55% | 6-methyl-2- (methylthio)-N-(3- (4- (trifluoromethoxy) phenyl)propyl) thieno[2, 3-d]pyrimidin- 4-amine |
| 125 | 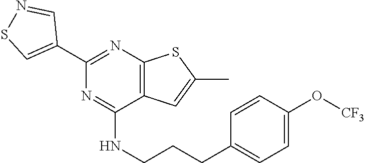 [SMILES: Cc1cc2c(NCCCc3ccc(OC(F)(F)F)cc3)nc(-c3cnsc3)nc2s1] | KH 7048 | Method A 1 h (140° C.) microwave | 12% | 2-(isothiazol-4-yl)-6- methyl-N-(3-(4- (trifluoromethoxy) phenyl)propyl)thieno [2,3-d]pyrimidin-4- amine |
| 126 | 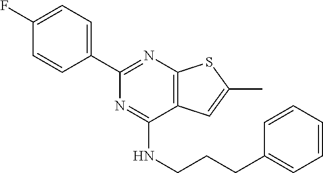 [SMILES: Cc1cc2c(NCCCc3ccccc3)nc(-c3ccc(F)cc3)nc2s1] | KH 5015 | Method A 1 h (140° C.) microwave | 67% | 2-(4-fluorophenyl)- 6-methyl-N-(3- phenylpropyl)thieno [2,3-d]pyrimidin-4-amine |
| 127 | 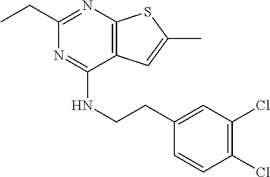 [SMILES: CCc1nc(NCCc2ccc(Cl)c(Cl)c2)c2cc(C)sc2n1] | KH 4007 | Method A 1 d (80° C.) | 24% | N-(3,4- dichlorophenethyl)- 2-ethyl-6- methylthieno[2,3- d]pyrimidin-4-amine |
| 128 | 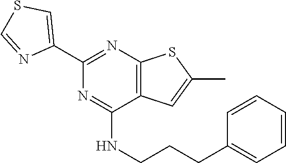 [SMILES: Cc1cc2c(NCCCc3ccccc3)nc(-c3cscn3)nc2s1] | KH 7076 | Method A 1 d (90° C.) | 63% | 6-methyl-N-(3- phenylpropyl)-2- (thiazol-4- yl)thieno[2,3- d]pyrimidin-4-amine |
| 129 | 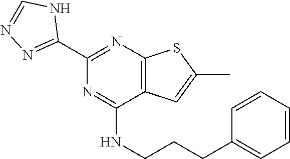 [SMILES: Cc1cc2c(NCCCc3ccccc3)nc(-c3nnc[nH]3)nc2s1] | KH 7077 | Method A 1 d (90° C.) | 53% | 6-methyl-N-(3- phenylpropyl)-2- (4H-1,2,4-triazol-3- yl)thieno[2,3- d]pyrimidin-4-amine |
| 130 | 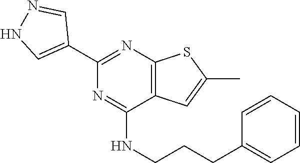 [SMILES: Cc1cc2c(NCCCc3ccccc3)nc(-c3cn[nH]c3)nc2s1] | KH 7078 | Method A 1 d (90° C.) | 37% | 6-methyl-N-(3- phenylpropyl)-2- (1H-pyrazol-4- yl)thieno[2,3- d]pyrimidin-4-amine |
| 131 | 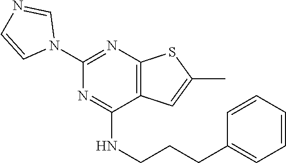 [SMILES: Cc1cc2c(NCCCc3ccccc3)nc(-n3ccnc3)nc2s1] | KH 7080 | Method A 1 d (90° C.) | 63% | 2-(1H-imidazol-1- yl)-6-methyl-N-(3- phenylpropyl)thieno [2,3-d]pyrimidin-4- amine |
| 132 | 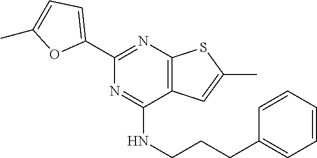 [SMILES: Cc1ccc(-c2nc(NCCCc3ccccc3)c3cc(C)sc3n2)o1] | KH 7081 | Method A 1 d (90° C.) | 73% | 6-methyl-2-(5- methylfuran-2-yl)-N- (3- phenylpropyl)thieno [2,3-d]pyrimidin-4- amine |
| 133 | 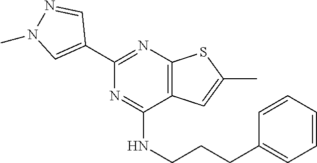 [SMILES: Cc1cc2c(NCCCc3ccccc3)nc(-c3cnn(C)c3)nc2s1] | KH 7079 | Method A 1 d (90° C.) | 40% | 6-methyl-2-(1- methyl-1H-pyrazol- 4-yl)-N-(3- phenylpropyl)thieno [2,3-d]pyrimidin-4- amine |
| 134 | 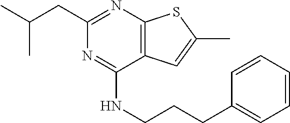 [SMILES: Cc1cc2c(NCCCc3ccccc3)nc(CC(C)C)nc2s1] | KH 8005 | Method A 1 d (90° C.) | 56% | 2-isobutyl-6-methyl- N-(3- phenylpropyl)thieno [2,3-d]pyrimidin-4- amine |
| 135 | 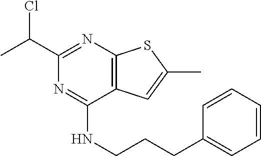 [SMILES: Cc1cc2c(NCCCc3ccccc3)nc(C(C)Cl)nc2s1] | KH 8006 | Method A 1 d (90° C.) | 57% | 2-(1-chloroethyl)-6- methyl-N-(3- phenylpropyl)thieno [2,3-d]pyrimidin-4- amine |
| 136 | 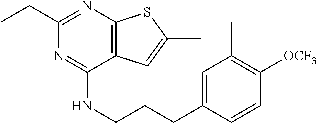 [SMILES: CCc1nc(NCCCc2ccc(OC(F)(F)F)c(C)c2)c2cc(C)sc2n1] | KH 4007 | Method C 2 h (140° C.) 1 h (150° C.) microwave | 8% | 2-ethyl-6-methyl-N- (3-(3-methyl-4- (trifluoromethoxy) phenyl)propyl)thieno [2,3-d]pyrimidin-4- amine |
| 137 | 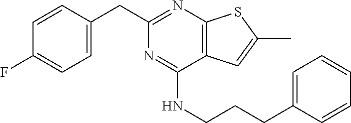 [SMILES: Cc1cc2c(NCCCc3ccccc3)nc(Cc3ccc(F)cc3)nc2s1] | KH 8019 | Method A 1 d (90° C.) | 42% | 2-(4-fluorobenzyl)-6- methyl-N-(3- phenylpropyl)thieno [2,3-d]pyrimidin-4- amine |
| 138 | 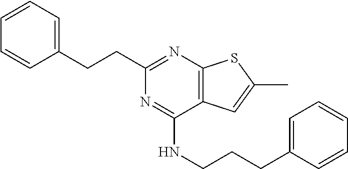 [SMILES: Cc1cc2c(NCCCc3ccccc3)nc(CCc3ccccc3)nc2s1] | KH 8020 | Method A 1 d (90° C.) | 56% | 6-methyl-2- phenethyl-N-(3- phenylpropyl)thieno [2,3-d]pyrimidin-4- amine |
| 139 | 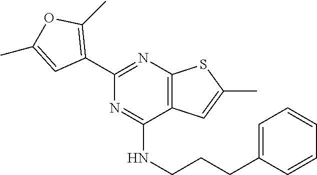 [SMILES: Cc1cc(-c2nc(NCCCc3ccccc3)c3cc(C)sc3n2)c(C)o1] | KH 8024 | Method A 1 d (90° C.) | 23% | 2-(2,5- dimethylfuran-3-yl)- 6-methyl-N-(3- phenylpropyl)thieno [2,3-d]pyrimidin-4- amine |
| 140 | 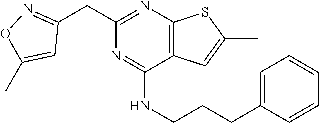 [SMILES: Cc1cc(Cc2nc(NCCCc3ccccc3)c3cc(C)sc3n2)no1] | KH 8029 | Method A 1 d (90° C.) | 70% | 6-methyl-2-((5- methylisoxazol-3- yl)methyl)-N-(3- phenylpropyl)thieno [2,3-d]pyrimidin-4- amine |
| 141 | 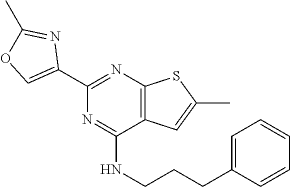 [SMILES: Cc1nc(-c2nc(NCCCc3ccccc3)c3cc(C)sc3n2)co1] | KH 8030 | Method A 1 h (140° C.) microwave | 43% | 6-methyl-2-(2- methyloxazol-4-yl)- N-(3- phenylpropyl)thieno [2,3-d]pyrimidin-4- amine |
| 142 | 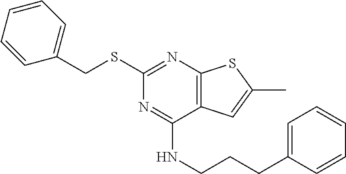 [SMILES: Cc1cc2c(NCCCc3ccccc3)nc(SCc3ccccc3)nc2s1] | KH 8031 | Method A 1 h (140° C.) microwave | 25% | 2-(benzylthio)-6- methyl-N-(3- phenylpropyl)thieno [2,3-d]pyrimidin-4- amine |
| 143 | 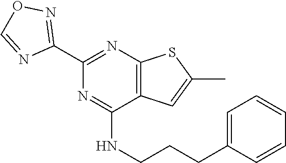 [SMILES: Cc1cc2c(NCCCc3ccccc3)nc(-c3ncon3)nc2s1] | KH 8054 | Method A 1 h (85° C.) | 71% | 6-methyl-2-(1,2,4- oxadiazol-3-yl)-N- (3- phenylpropyl)thieno [2,3-d]pyrimidin-4- amine |
| 144 | 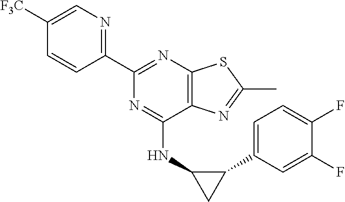 [SMILES: Cc1nc2c(N[C@@H]3C[C@H]3c3ccc(F)c(F)c3)nc(-c3ccc(C(F)(F)F)cn3)nc2s1] | LM 8070 | Method A 2 h (150° C.) microwave | 90% | N-((1R,2S)-2-(3,4- difluorophenyl) cyclopropyl)-2- methyl-5-(5- (trifluoromethyl) pyridin-2- yl)thiazolo[5,4- d]pyrimidin-7-amine |
| 145 | 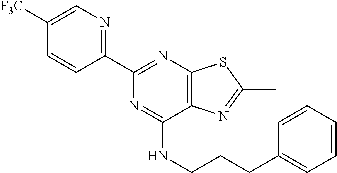 [SMILES: Cc1nc2c(NCCCc3ccccc3)nc(-c3ccc(C(F)(F)F)cn3)nc2s1] | LM 8070 | Method A 2 h (150° C.) microwave | 62% | 2-methyl-N-(3- phenylpropyl)-5-(5- (trifluoromethyl) pyridin-2- yl)thiazolo[5,4- d]pyrimidin-7-amine |
| 146 | 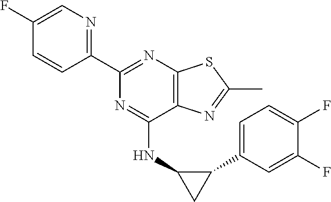 [SMILES: Cc1nc2c(N[C@@H]3C[C@H]3c3ccc(F)c(F)c3)nc(-c3ccc(F)cn3)nc2s1] | LM 8071 | Method A 2 h (150° C.) microwave | 55% | N-((1R,2S)-2-(3,4- difluorophenyl) cyclopropyl)-5-(5- fluoropyridin-2-yl)- 2- methylthiazolo[5,4- d]pyrimidin-7-amine |
| 147 | 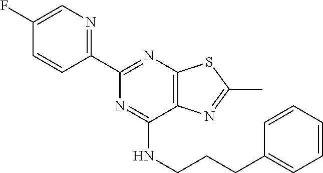 [SMILES: Cc1nc2c(NCCCc3ccccc3)nc(-c3ccc(F)cn3)nc2s1] | LM 8071 | Method A 2 h (150° C.) microwave | 45% | 5-(5-fluoropyridin-2- yl)-2-methyl-N-(3- phenylpropyl)thiazolo [5,4-d]pyrimidin-7- amine |
| 148 | 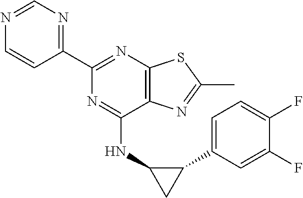 [SMILES: Cc1nc2c(N[C@@H]3C[C@H]3c3ccc(F)c(F)c3)nc(-c3ccncn3)nc2s1] | LM 8073 | Method A 2 h (150° C.) microwave | 23% | N-((1R,2S)-2-(3,4- difluorophenyl) cyclopropyl)-2- methyl-5-(pyrimidin- 4-yl)thiazolo[5,4- d]pyrimidin-7-amine |
| 149 | 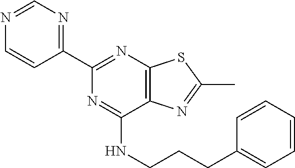 [SMILES: Cc1nc2c(NCCCc3ccccc3)nc(-c3ccncn3)nc2s1] | LM 8073 | Method A 2 h (150° C.) microwave | 48% | 2-methyl-N-(3- phenylpropyl)-5- (pyrimidin-4- yl)thiazolo[5,4- d]pyrimidin-7-amine |
| 150 | 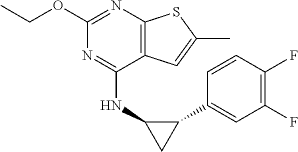 [SMILES: CCOc1nc(N[C@@H]2C[C@H]2c2ccc(F)c(F)c2)c2cc(C)sc2n1] | LM 9031 | Method A 4 h (120° C.) microwave | 17% | N-((1R,2S)-2-(3,4- difluorophenyl) cyclopropyl)-2-ethoxy- 6-methylthieno[2,3- d]pyrimidin-4-amine |
| 151 | 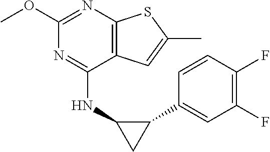 [SMILES: COc1nc(N[C@@H]2C[C@H]2c2ccc(F)c(F)c2)c2cc(C)sc2n1] | LM 9038 | Method A 1 h (140° C.) microwave | 27% | N-((1R,2S)-2-(3,4- difluorophenyl) cyclopropyl)- 2-methoxy- 6-methylthieno[2,3- d]pyrimidin-4-amine |
| 152 | 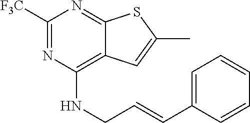 [SMILES: Cc1cc2c(NC/C=C/c3ccccc3)nc(C(F)(F)F)nc2s1] | KH 6080 | Method A 16 h (25° C.) | 55% | N-cinnamyl-6- methyl-2- (trifluoromethyl) thieno[2,3-d] pyrimidin-4-amine |
| 153 | 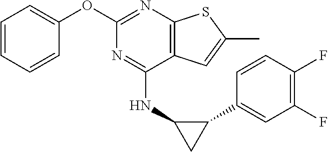 [SMILES: Cc1cc2c(N[C@@H]3C[C@H]3c3ccc(F)c(F)c3)nc(Oc3ccccc3)nc2s1] | LM 9074 | Method A 3 h (140° C.) microwave | 76% | N-((1R,2S)-2-(3,4- difluorophenyl) cyclopropyl)-6-methyl- 2-phenoxythieno[2,3- d]pyrimidin-4-amine |
| 154 | 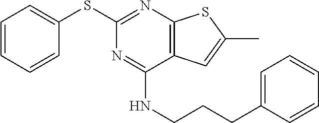 [SMILES: Cc1cc2c(NCCCc3ccccc3)nc(Sc3ccccc3)nc2s1] | LM 9078 | Method A 1 h (150° C.) microwave | 40% | 6-methyl-N-(3- phenylpropyl)-2- (phenylthio)thieno[2, 3-d]pyrimidin-4- amine |
| 155 | 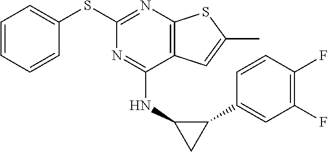 [SMILES: Cc1cc2c(N[C@@H]3C[C@H]3c3ccc(F)c(F)c3)nc(Sc3ccccc3)nc2s1] | LM 9078 | Method A 2 h (140° C.) microwave | 53% | N-((1R,2S)-2-(3,4- difluorophenyl) cyclopropyl)-6-methyl- 2-(phenylthio)thieno[2, 3-d]pyrimidin-4- amine |
| 156 | 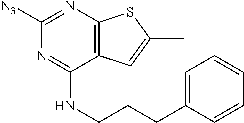 [SMILES: Cc1cc2c(NCCCc3ccccc3)nc(N=[N+]=[N-])nc2s1] | LM 9044 | Method A 1 d (25° C.) | 17% | 2-azido-6-methyl-N- (3- phenylpropyl)thieno [2,3-d]pyrimidin-4- amine |
| 157 | 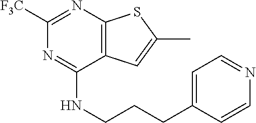 [SMILES: Cc1cc2c(NCCCc3ccncc3)nc(C(F)(F)F)nc2s1] | KH 6080 | Method A 3 d (25° C.) | 60% | 6-methyl-N-(3- (pyridin-4-yl)propyl)- 2-(trifluoromethyl) thieno[2,3-d] pyrimidin- 4-amine |
| 158 | 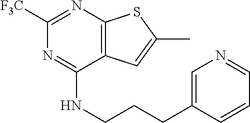 [SMILES: Cc1cc2c(NCCCc3cccnc3)nc(C(F)(F)F)nc2s1] | KH 6080 | Method A 3 d (25° C.) | 56% | 6-methyl-N-(3- (pyridin-3- yl)propyl)-2- (trifluoromethyl) thieno[2,3-d] pyrimidin-4-amine |
| 159 | 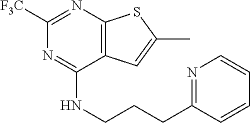 [SMILES: Cc1cc2c(NCCCc3ccccn3)nc(C(F)(F)F)nc2s1] | KH 6080 | Method A 16 h (25° C.) | 87% | 6-methyl-N-(3- (pyridin-2- yl)propyl)-2- (trifluoromethyl)thieno [2,3-d]pyrimidin- 4-amine |
| 160 | 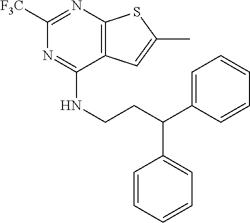 [SMILES: Cc1cc2c(NCCC(c3ccccc3)c3ccccc3)nc(C(F)(F)F)nc2s1] | KH 6080 | Method A 16 h (25° C.) | 20% | N-(3,3- diphenylpropyl)-6- methyl-2- (trifluoromethyl) thieno[2,3-d] pyrimidin-4-amine |
| 161 | 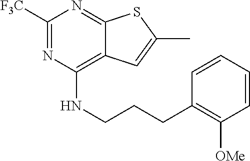 [SMILES: COc1ccccc1CCCNc1nc(C(F)(F)F)nc2sc(C)cc12] | KH 6080 | Method A 2 d (25° C.) | 87% | N-(3-(2- methoxyphenyl)propyl)- 6-methyl-2- (trifluoromethyl) thieno[2,3-d]pyrimidin- 4-amine |
| 162 | 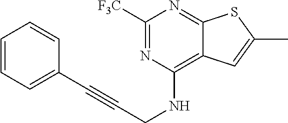 [SMILES: Cc1cc2c(NCC#Cc3ccccc3)nc(C(F)(F)F)nc2s1] | KH 6080 | Method A 16 h (25° C.) | 49% | 6-methyl-N-(3- phenylprop-2-yn-1- yl)-2-(trifluoromethyl) thieno[2,3-d] pyrimidin- 4-amine |
| 163 | 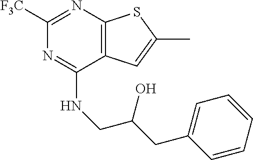 [SMILES: Cc1cc2c(NCC(O)Cc3ccccc3)nc(C(F)(F)F)nc2s1] | KH 6080 | Method A 16 h (25° C.) | 48% | 1-((6-methyl-2- (trifluoromethyl) thieno[2,3-d]pyrimidin- 4-yl)amino)-3- phenylpropan-2-ol |
| 164 | 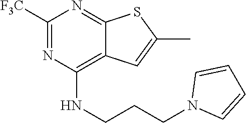 [SMILES: Cc1cc2c(NCCCn3cccc3)nc(C(F)(F)F)nc2s1] | KH 6080 | Method A 16 h (25° C.) | 79% | N-(3-(1H-pyrrol-1- yl)propyl)-6-methyl- 2-(trifluoromethyl) thieno[2,3-d] pyrimidin- 4-amine |
| 165 | 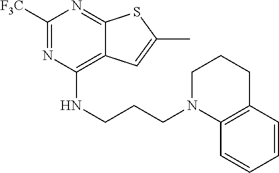 [SMILES: Cc1cc2c(NCCCN3CCCc4ccccc43)nc(C(F)(F)F)nc2s1] | KH 6080 | Method A 16 h (25° C.) | 64% | N-(3-(3,4- dihydroquinolin- 1(2H)-yl)propyl)-6- methyl-2- (trifluoromethyl) thieno[2,3-d] pyrimidin-4-amine |
| 166 | 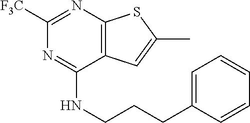 [SMILES: Cc1cc2c(NCCCc3ccccc3)nc(C(F)(F)F)nc2s1] | KH 6080 | Method A 16 h (25° C.) | 99% | 6-methyl-N-(3- phenylpropyl)-2- (trifluoromethyl) thieno[2,3-d] pyrimidin-4-amine |
| 167 | 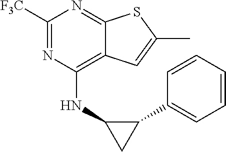 [SMILES: Cc1cc2c(N[C@@H]3C[C@H]3c3ccccc3)nc(C(F)(F)F)nc2s1] | KH 6080 | Method A 16 h (25° C.) | 69% | 6-methyl-N- ((1R,2S)-2- phenylcyclopropyl)- 2-(trifluoromethyl) thieno[2,3-d] pyrimidin- 4-amine |
| 168 | 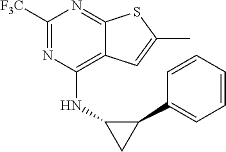 [SMILES: Cc1cc2c(N[C@H]3C[C@@H]3c3ccccc3)nc(C(F)(F)F)nc2s1] | KH 6080 | Method A 16 h (25° C.) | 77% | 6-methyl-N- ((1S,2R)-2- phenylcyclopropyl)- 2-(trifluoromethyl) thieno[2,3-d] pyrimidin- 4-amine |
| 169 | 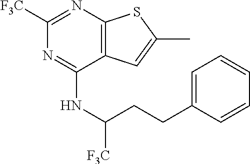 [SMILES: Cc1cc2c(NC(CCc3ccccc3)C(F)(F)F)nc(C(F)(F)F)nc2s1] | KH 6080 | Method A 5 h (160° C.) microwave | 10% | 6-methyl-N-(1,1,1- trifluoro-4- phenylbutan-2-yl)-2- (trifluoromethyl) thieno[2,3-d] pyrimidin-4-amine |
| 170 | 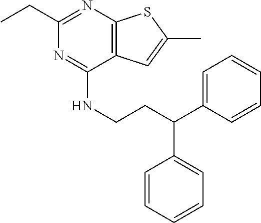 [SMILES: CCc1nc(NCCC(c2ccccc2)c2ccccc2)c2cc(C)sc2n1] | KH 4007 | Method A 2 d (85° C.) | 25% | N-(3,3- diphenylpropyl)-2- ethyl-6- methylthieno[2,3- d]pyrimidin-4-amine |
| 171 | 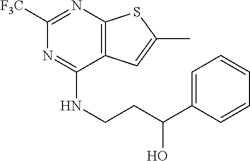 [SMILES: Cc1cc2c(NCCC(O)c3ccccc3)nc(C(F)(F)F)nc2s1] | KH 6080 | Method A 16 h (25° C.) | 78% | 3-((6-methyl-2- (trifluoromethyl) thieno [2,3-d]pyrimidin- 4-yl)amino)-1- phenylpropan-1-ol |
| 172 | 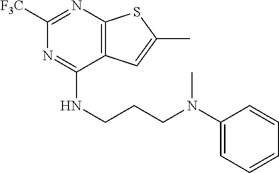 [SMILES: Cc1cc2c(NCCCN(C)c3ccccc3)nc(C(F)(F)F)nc2s1] | KH 6080 | Method A 16 h (25° C.) | 81% | N1-methyl-N3-(6- methyl-2- (trifluoromethyl) thieno[2,3-d] pyrimidin-4-yl)-N1- phenylpropane-1,3- diamine |
| 173 | 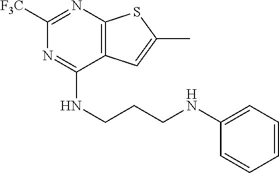 [SMILES: Cc1cc2c(NCCCNc3ccccc3)nc(C(F)(F)F)nc2s1] | KH 6080 | Method A 7 d (25° C.) | 40% | N1-(6-methyl-2- (trifluoromethyl) thieno[2,3-d] pyrimidin-4-yl)-N3- phenylpropane-1,3- diamine |
| 175 | 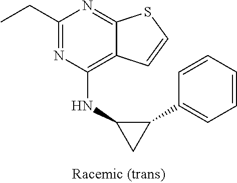 [SMILES: CCc1nc(N[C@@H]2C[C@H]2c2ccccc2)c2ccsc2n1] | KH 4018 | Method A 1 h (140° C.) microwave | 29% | 2-ethyl-N-((rac- 1R,2S)-2- phenylcyclopropyl) thieno[2,3- d]pyrimidin-4-amine |
| 176 | 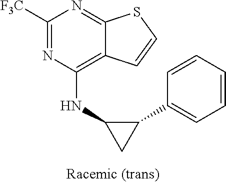 [SMILES: FC(F)(F)c1nc(N[C@@H]2C[C@H]2c2ccccc2)c2ccsc2n1] | KH 6080 | Method A 1 h (140° C.) microwave | 77% | 6-methyl-N-((rac- 1R,2S)-2- phenylcyclopropyl)- 2-(trifluoromethyl) thieno[2,3-d] pyrimidin- 4-amine |
| 177 | 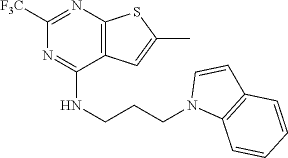 [SMILES: Cc1cc2c(NCCCn3ccc4ccccc43)nc(C(F)(F)F)nc2s1] | KH 6080 | Method A 3 d (25° C.) | 53% | N-(3-(1H-indol-1- yl)propyl)-6-methyl- 2-(trifluoromethyl) thieno[2,3-d] pyrimidin- 4-amine |
| 178 | 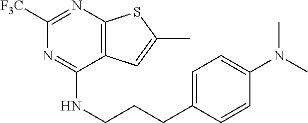 [SMILES: Cc1cc2c(NCCCc3ccc(N(C)C)cc3)nc(C(F)(F)F)nc2s1] | KH 6080 | Method A 16 h (25° C.) | 66% | N-(3-(4- (dimethylamino) phenyl)propyl)-6- methyl-2- (trifluoromethyl) thieno[2,3-d] pyrimidin-4-amine |
| 179 | 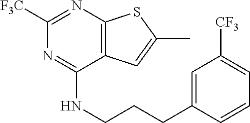 [SMILES: Cc1cc2c(NCCCc3cccc(C(F)(F)F)c3)nc(C(F)(F)F)nc2s1] | KH 6080 | Method A 16 h (25° C.) | 41% | 6-methyl-2- (trifluoromethyl)-N- (3-(3- (trifluoromethyl) phenyl)propyl) thieno[2,3-d] pyrimidin-4-amine |
| 180 | 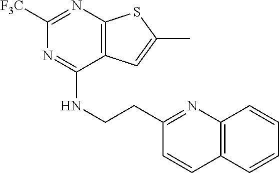 [SMILES: Cc1cc2c(NCCc3ccc4ccccc4n3)nc(C(F)(F)F)nc2s1] | KH 6080 | Method A 16 h (25° C.) | 35% | 6-methyl-N-(2- (quinolin-2-yl)ethyl)- 2-(trifluoromethyl) thieno[2,3-d] pyrimidin-4-amine |
| 181 | 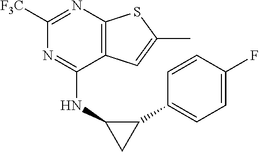 | KH 6080 | Method A 16 h (25° C.) | 21% | N-((1R,2S)-2-(4- fluorophenyl) cyclopropyl)- 6-methyl-2- (trifluoromethyl) thieno[2,3-d] pyrimidin-4-amine |
| 182 | 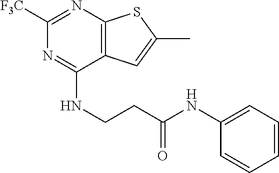 [SMILES: Cc1cc2c(NCCC(=O)Nc3ccccc3)nc(C(F)(F)F)nc2s1] | KH 6080 | Method A 16 h (25° C.) | 70% | 3-((6-methyl-2- (trifluoromethyl) thieno[2,3-d]pyrimidin- 4-yl)amino)-N- phenylpropanamide |
| 183 | 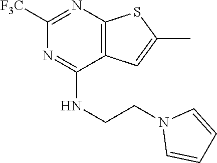 [SMILES: Cc1cc2c(NCCn3cccc3)nc(C(F)(F)F)nc2s1] | KH 6080 | Method A 16 h (25° C.) | 77% | N-(2-(1H-pyrrol-1- yl)ethyl)-6-methyl-2- (trifluoromethyl) thieno[2,3-d]pyrimidin- 4-amine |
| 184 | 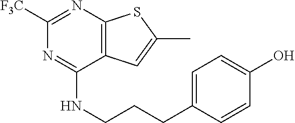 [SMILES: Cc1cc2c(NCCCc3ccc(O)cc3)nc(C(F)(F)F)nc2s1] | KH 6080 | Method A 2 d (25° C.) | 60% | 4-(3-((6-methyl-2- (trifluoromethyl) thieno[2,3-d]pyrimidin- 4- yl)amino)propyl) phenol |
| 185 | 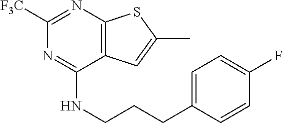 [SMILES: Cc1cc2c(NCCCc3ccc(F)cc3)nc(C(F)(F)F)nc2s1] | KH 6080 | Method A 2 d (25° C.) | 99% | N-(3-(4- fluorophenyl) propyl)-6-methyl- 2-(trifluoromethyl) thieno[2,3-d] pyrimidin-4-amine |
| 186 | 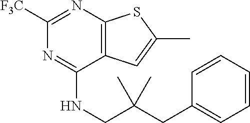 [SMILES: Cc1cc2c(NCC(C)(C)Cc3ccccc3)nc(C(F)(F)F)nc2s1] | KH 6080 | Method A 16 h (25° C.) | 61% | N-(2,2-dimethyl-3- phenylpropyl)-6- methyl-2- (trifluoromethyl) thieno[2,3-d] pyrimidin-4-amine |
| 187 | 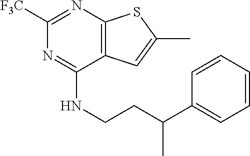 [SMILES: Cc1cc2c(NCCC(C)c3ccccc3)nc(C(F)(F)F)nc2s1] | KH 6080 | Method A 16 h (25° C.) | 93% | 6-methyl-N-(3- phenylbutyl)-2- (trifluoromethyl) thieno[2,3-d] pyrimidin-4-amine |
| 188 | 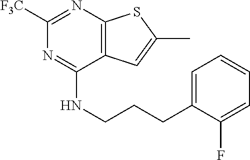 [SMILES: Cc1cc2c(NCCCc3ccccc3F)nc(C(F)(F)F)nc2s1] | KH 6080 | Method A 16 h (25° C.) | 78% | N-(3-(2- fluorophenyl)propyl)- 6-methyl-2- (trifluoromethyl) thieno[2,3-d] pyrimidin-4-amine |
| 189 | 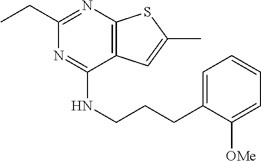 [SMILES: CCc1nc(NCCCc2ccccc2OC)c2cc(C)sc2n1] | KH 4007 | Method A 1 h (140° C.) microwave | 48% | 2-ethyl-N-(3-(2- methoxyphenyl) propyl)-6- methylthieno[2,3- d]pyrimidin-4-amine |
| 190 | 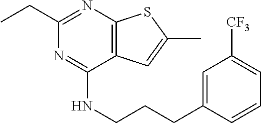 [SMILES: CCc1nc(NCCCc2cccc(C(F)(F)F)c2)c2cc(C)sc2n1] | KH 4007 | Method A 1 h (140° C.) microwave | 30% | 2-ethyl-6-methyl-N- (3-(3- (trifluoromethyl) phenyl)propyl) thieno[2,3-d] pyrimidin-4-amine |
| 191 | 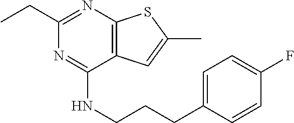 [SMILES: CCc1nc(NCCCc2ccc(F)cc2)c2cc(C)sc2n1] | KH 4007 | Method A 1 h (140° C.) microwave | 62% | 2-ethyl-N-(3-(4- fluorophenyl)propyl)- 6-methylthieno[2,3- d]pyrimidin-4-amine |
| 192 | 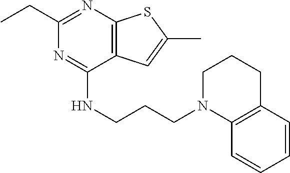 [SMILES: CCc1nc(NCCCN2CCCc3ccccc32)c2cc(C)sc2n1] | KH 4007 | Method A 1 h (140° C.) microwave | 93% | N-(3-(3,4- dihydroquinolin- 1(2H)-yl)propyl)-2- ethyl-6- methylthieno[2,3- d]pyrimidin-4-amine |
| 193 | 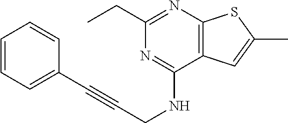 [SMILES: CCc1nc(NCC#Cc2ccccc2)c2cc(C)sc2n1] | KH 4007 | Method A 1 h (140° C.) microwave | 57% | 2-ethyl-6-methyl-N- (3-phenylprop-2-yn- 1-yl)thieno[2,3- d]pyrimidin-4-amine |
| 194 | 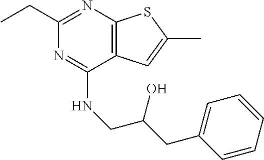 [SMILES: CCc1nc(NCC(O)Cc2ccccc2)c2cc(C)sc2n1] | KH 4007 | Method A 2 d (40° C.) | 80% | 1-((2-ethyl-6- methylthieno[2,3- d]pyrimidin-4- yl)amino)-3- phenylpropan-2-ol |
| 195 | 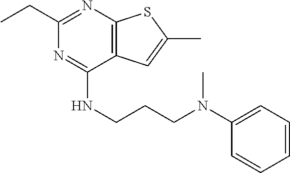 [SMILES: CCc1nc(NCCCN(C)c2ccccc2)c2cc(C)sc2n1] | KH 4007 | Method A 1 h (140° C.) microwave | 50% | N1-(2-ethyl-6- methylthieno[2,3- d]pyrimidin-4-yl)- N3-methyl-N3- phenylpropane-1,3- diamine |
| 196 | 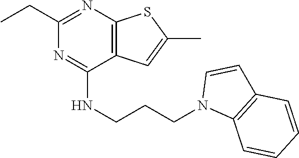 [SMILES: CCc1nc(NCCCn2ccc3ccccc32)c2cc(C)sc2n1] | KH 4007 | Method A 4 h (140° C.) microwave | 36% | N-(3-(1H-indol-1- yl)propyl)-2-ethyl-6- methylthieno[2,3- d]pyrimidin-4-amine |
| 197 | 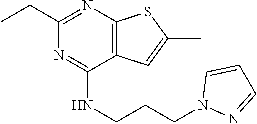 [SMILES: CCc1nc(NCCCn2cccn2)c2cc(C)sc2n1] | KH 4007 | Method A 1 h (140° C.) microwave | 51% | N-(3-(1H-pyrazol-1- yl)propyl)-2-ethyl-6- methylthieno[2,3- d]pyrimidin-4-amine |
| 198 | 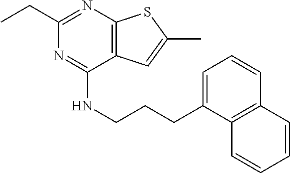 [SMILES: CCc1nc(NCCCc2cccc3ccccc23)c2cc(C)sc2n1] | KH 4007 | Method A 1 h (140° C.), 2 h (160° C.) microwave | 38% | 2-ethyl-6-methyl-N- (3-(naphthalen-1- yl)propyl)thieno[2,3- d]pyrimidin-4-amine |
| 199 | 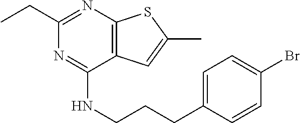 [SMILES: CCc1nc(NCCCc2ccc(Br)cc2)c2cc(C)sc2n1] | KH 4007 | Method A 5 d (40° C.) | 85% | N-(3-(4- bromophenyl)propyl)- 2-ethyl-6- methylthieno[2,3- d]pyrimidin-4-amine |
| 200 | 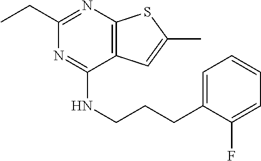 [SMILES: CCc1nc(NCCCc2ccccc2F)c2cc(C)sc2n1] | KH 4007 | Method A 16 h (40° C.) | 33% | 2-ethyl-N-(3-(2- fluorophenyl)propyl)- 6-methylthieno[2,3- d]pyrimidin-4-amine |
| 201 | 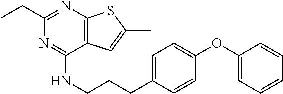 [SMILES: CCc1nc(NCCCc2ccc(Oc3ccccc3)cc2)c2cc(C)sc2n1] | KH 4007 | Method A 1 h (140° C.) microwave | 58% | 2-ethyl-6-methyl-N- (3-(4- phenoxyphenyl) propyl)thieno[2,3- d]pyrimidin-4-amine |
| 202 | 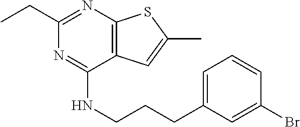 [SMILES: CCc1nc(NCCCc2cccc(Br)c2)c2cc(C)sc2n1] | KH 4007 | Method A 1 d (60° C.) 3 h (80° C.) | 69% | N-(3-(3- bromophenyl) propyl)-2-ethyl-6- methylthieno[2,3- d]pyrimidin-4-amine |
| 203 | 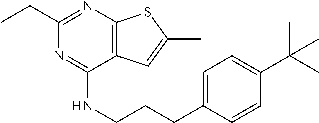 [SMILES: CCc1nc(NCCCc2ccc(C(C)(C)C)cc2)c2cc(C)sc2n1] | KH 4007 | Method A 1 h (150° C.) microwave | 74% | N-(3-(4-(tert- butyl)phenyl) propyl)-2-ethyl-6- methylthieno[2,3- d]pyrimidin-4-amine |
| 204 | 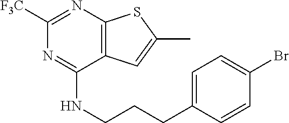 [SMILES: Cc1cc2c(NCCCc3ccc(Br)cc3)nc(C(F)(F)F)nc2s1] | KH 6080 | Method A 1 h (40° C.) | 99% | N-(3-(4- bromophenyl) propyl)-6-methyl-2- (trifluoromethyl) thieno[2,3-d] pyrimidin-4-amine |
| 205 | 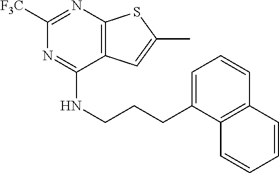 [SMILES: Cc1cc2c(NCCCc3cccc4ccccc34)nc(C(F)(F)F)nc2s1] | KH 6080 | Method A 1 h (80° C.) | 30% | 6-methyl-N-(3- (naphthalen-1- yl)propyl)-2- (trifluoromethyl) thieno[2,3-d] pyrimidin-4-amine |
| 206 | 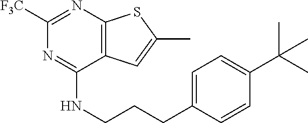 [SMILES: Cc1cc2c(NCCCc3ccc(C(C)(C)C)cc3)nc(C(F)(F)F)nc2s1] | KH 6080 | Method A 2 h (60° C.) | 85% | N-(3-(4-(tert- butyl)phenyl)propyl)- 6-methyl-2- (trifluoromethyl) thieno[2,3-d] pyrimidin-4-amine |
| 207 | 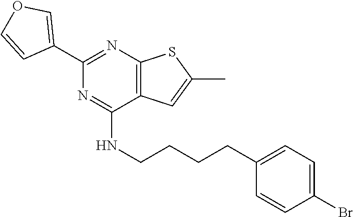 [SMILES: Cc1cc2c(NCCCCc3ccc(Br)cc3)nc(-c3ccoc3)nc2s1] | KH6067 | Method A 1 h (140° C.) microwave | 67% | N-(4-(4- bromophenyl)butyl)- 2-(furan-3-yl)-6- methylthieno[2,3- d]pyrimidin-4-amine |
| 208 | 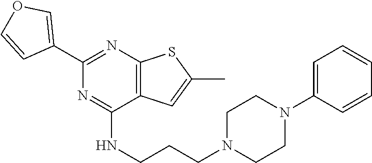 [SMILES: Cc1cc2c(NCCCN3CCN(c4ccccc4)CC3)nc(-c3ccoc3)nc2s1] | KH6067 | Method A 1 d (90° C.) | 53% | 2-(furan-3-yl)-6- methyl-N-(3-(4- phenylpiperazin-1- yl)propyl)thieno[2,3- d]pyrimidin-4-amine |
| 209 | 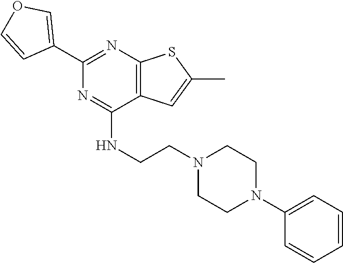 [SMILES: Cc1cc2c(NCCN3CCN(c4ccccc4)CC3)nc(-c3ccoc3)nc2s1] | KH6067 | Method A 1 d (90° C.) | 74% | 2-(furan-3-yl)-6- methyl-N-(2-(4- phenylpiperazin-1- yl)ethyl)thieno[2,3- d]pyrimidin-4-amine |
| 210 | 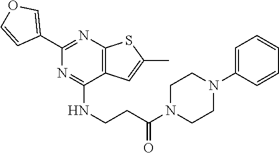 [SMILES: Cc1cc2c(NCCC(=O)N3CCN(c4ccccc4)CC3)nc(-c3ccoc3)nc2s1] | KH6067 | Method A 1 d (90° C.) | 50% | 3-((2-(furan-3-yl)-6- methylthieno[2,3- d]pyrimidin-4- yl)amino)-1-(4- phenylpiperazin-1- yl)propan-1-one |
| 211 | 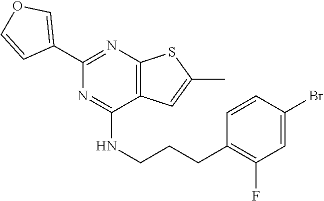 [SMILES: Cc1cc2c(NCCCc3ccc(Br)cc3F)nc(-c3ccoc3)nc2s1] | KH6067 | Method A 1 h (140° C.) microwave | 48% | N-(3-(4-bromo-2- fluorophenyl)propyl)- 2-(furan-3-yl)-6- methylthieno[2,3- d]pyrimidin-4-amine |
| 212 | 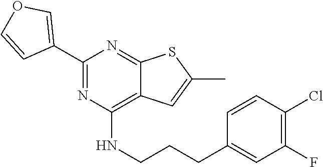 [SMILES: Cc1cc2c(NCCCc3ccc(Cl)c(F)c3)nc(-c3ccoc3)nc2s1] | KH6067 | Method A 2 d (70° C.) | 73% | N-(3-(4 −chloro-3- fluorophenyl)propyl)- 2-(furan-3-yl)-6- methylthieno[2,3- d]pyrimidin-4-amine |
| 213 | 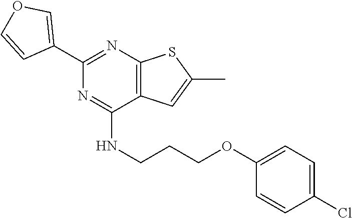 [SMILES: Cc1cc2c(NCCCOc3ccc(Cl)cc3)nc(-c3ccoc3)nc2s1] | KH6067 | Method A 1 h (140° C.) microwave | 38% | N-(3-(4- chlorophenoxy)propyl)- 2-(furan-3-yl)-6- methylthieno[2,3- d]pyrimidin-4-amine |
| 214 | 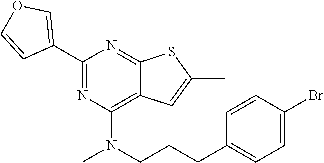 [SMILES: Cc1cc2c(N(C)CCCc3ccc(Br)cc3)nc(-c3ccoc3)nc2s1] | KH6067 | Method A 1 d (100° C.) | 48% | N-(3-(4- bromophenyl)propyl)- 2-(furan-3-yl)-N,6- dimethylthieno[2,3- d]pyrimidin-4-amine |
| 215 | 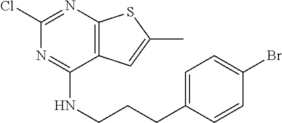 [SMILES: Cc1cc2c(NCCCc3ccc(Br)cc3)nc(Cl)nc2s1] | 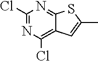 purchased | Method A 16 h (60° C.) | 96% | N-(3-(4- bromophenyl)propyl)- 2-chloro-6- methylthieno[2,3- d]pyrimidin-4-amine |
| 216 | 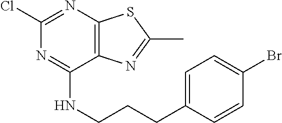 [SMILES: Cc1nc2c(NCCCc3ccc(Br)cc3)nc(Cl)nc2s1] | 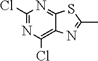 purchased | Method A 3 d (60° C.) | 80% | N-(3-(4- bromophenyl)propyl)- 5-chloro-2- methylthiazolo[5,4- d]pyrimidin-7-amine |
| 217 | 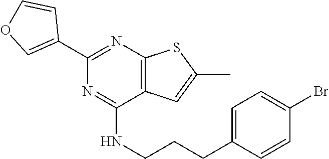 [SMILES: Cc1cc2c(NCCCc3ccc(Br)cc3)nc(-c3ccoc3)nc2s1] | KH 6067 | Method A 3 d (60° C.) 6 h (90° C.) | 89% | N-(3-(4- bromophenyl)propyl)- 2-(furan-3-yl)-6- methylthieno[2,3- d]pyrimidin-4-amine |
| 218 | 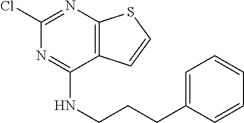 [SMILES: Clc1nc(NCCCc2ccccc2)c2ccsc2n1] | 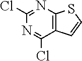 [SMILES: Clc1nc(Cl)c2ccsc2n1] purchased | Method B 2 h (25° C.) | 78% | 2-chloro-N-(3- phenylpropyl)thieno [2,3-d]pyrimidin-4- amine |
| 219 | 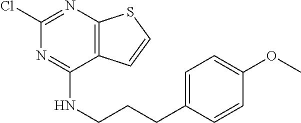 [SMILES: COc1ccc(CCCNc2nc(Cl)nc3sccc23)cc1] | 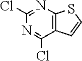 [SMILES: Clc1nc(Cl)c2ccsc2n1] purchased | Method B 2 h (25° C.) | 67% | 2-chloro-N-(3-(4- methoxyphenyl) propyl)thieno[2,3- d]pyrimidin-4-amine |
| 220 | 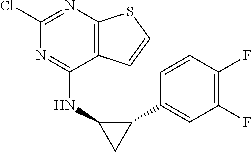 [SMILES: Fc1ccc([C@@H]2C[C@H]2Nc2nc(Cl)nc3sccc23)cc1F] | 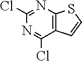 [SMILES: Clc1nc(Cl)c2ccsc2n1] purchased | Method B 2 h (25° C.) | 67% | 2-chloro-N-((1R,2S)- 2-(3,4- difluorophenyl) cyclopropyl)thieno[2,3- d]pyrimidin-4-amine |
| 221 | 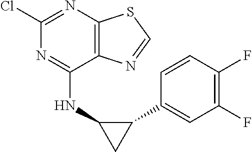 [SMILES: Fc1ccc([C@@H]2C[C@H]2Nc2nc(Cl)nc3scnc23)cc1F] | 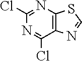 [SMILES: Clc1nc(Cl)c2ncsc2n1] purchased | Method B 3 h (25° C.) | 42% | 5-chloro-N-((1R,2S)- 2-(3,4- difluorophenyl) cyclopropyl)thiazolo [5,4-d]pyrimidin- 7-amine |
| 222 | 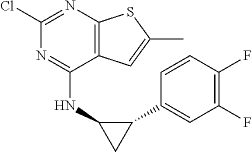 [SMILES: Cc1cc2c(N[C@@H]3C[C@H]3c3ccc(F)c(F)c3)nc(Cl)nc2s1] | 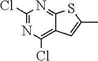 purchased | Method B 3 h (25° C.) | 52% | 2-chloro-N-((1R,2S)- 2-(3,4- difluorophenyl) cyclopropyl)-6- methylthieno[2,3- d]pyrimidin-4-amine |
| 223 | 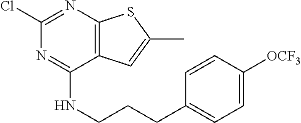 [SMILES: Cc1cc2c(NCCCc3ccc(OC(F)(F)F)cc3)nc(Cl)nc2s1] | 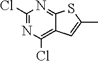 purchased | Method B 3 h (25° C.) | 78% | 2-chloro-6-methyl- N-(3-(4- (trifluoromethoxy) phenyl)propyl) thieno[2,3-d] pyrimidin-4-amine |
| 224 | 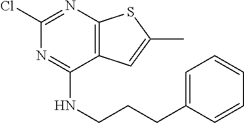 [SMILES: Cc1cc2c(NCCCc3ccccc3)nc(Cl)nc2s1] | 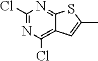 purchased | Method B 1 d (25° C.) | 81% | 2-chloro-6-methyl- N-(3- phenylpropyl)thieno [2,3-d]pyrimidin-4- amine |
| 225 | 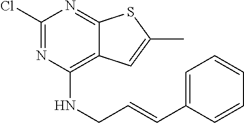 [SMILES: Cc1cc2c(NC/C=C/c3ccccc3)nc(Cl)nc2s1] | 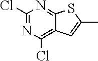 purchased | Method B 1 d (25° C.) | 89% | 2-chloro-N- cinnamyl-6- methylthieno[2,3- d]pyrimidin-4- amine |
| 226 | 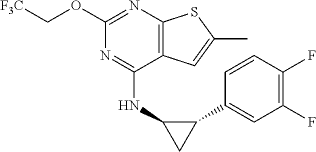 [SMILES: Cc1cc2c(N[C@@H]3C[C@H]3c3ccc(F)c(F)c3)nc(OCC(F)(F)F)nc2s1] | LM 9048 | Method B 1 d (40° C.) | 39% | N-((1R,2S)-2-(3,4- difluorophenyl) cyclopropyl)-6- methyl-2-(2,2,2- trifluoroethoxy)thieno [2,3-d]pyrimidin-4- amine |
| 227 | 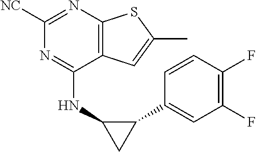 [SMILES: Cc1cc2c(N[C@@H]3C[C@H]3c3ccc(F)c(F)c3)nc(C#N)nc2s1] | LM 9061 | Method B 1 d (25° C.) | 31% | 4-(((1R,2S)-2-(3,4- difluorophenyl) cyclopropyl) amino)-6- methylthieno[2,3- d]pyrimidine-2- carbonitrile |
| 228 | 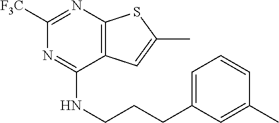 [SMILES: Cc1cccc(CCCNc2nc(C(F)(F)F)nc3sc(C)cc23)c1] | KH 6080 | Method C 2 d (25° C.) | 58% | 6-methyl-N-(3-(m- tolyl)propyl)-2- (trifluoromethyl)thieno [2,3-d]pyrimidin- 4-amine |
| 229 | 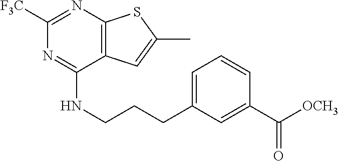 [SMILES: COC(=O)c1cccc(CCCNc2nc(C(F)(F)F)nc3sc(C)cc23)c1] | KH 6080 | Method C 2 d (25° C.) | 22% | methyl 3-(3-((6- methyl-2- (trifluoromethyl)thieno [2,3-d]pyrimidin- 4-yl)amino)propyl) benzoate |
| 230 | 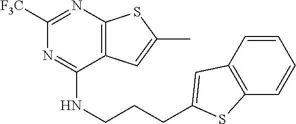 [SMILES: Cc1cc2c(NCCCc3cc4ccccc4s3)nc(C(F)(F)F)nc2s1] | KH 6080 | Method C 2 d (25° C.) | 29% | N-(3- (benzo [b]thiophen-2- yl)propyl)-6-methyl- 2-(trifluoromethyl) thieno [2,3-d]pyrimidin- 4-amine |
| 231 | 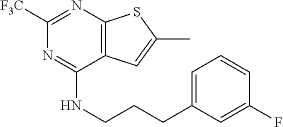 [SMILES: Cc1cc2c(NCCCc3cccc(F)c3)nc(C(F)(F)F)nc2s1] | KH 6080 | Method C 2 d (25° C.) | 49% | N-(3-(3- fluorophenyl)propyl)- 6-methyl-2- (trifluoromethyl)thieno [2,3-d]pyrimidin- 4-amine |
| 232 | 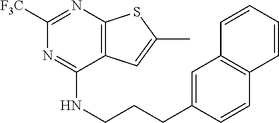 [SMILES: Cc1cc2c(NCCCc3ccc4ccccc4c3)nc(C(F)(F)F)nc2s1] | KH 6080 | Method C 2 d (25° C.) | 56% | 6-methyl-N-(3- (naphthalen-2- yl)propyl)-2- (trifluoromethyl) thieno[2,3-d] pyrimidin-4-amine |
| 233 | 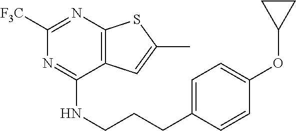 [SMILES: Cc1cc2c(NCCCc3ccc(OC4CC4)cc3)nc(C(F)(F)F)nc2s1] | KH 6080 | Method C 2 d (25° C.) | 14% | N-(3-(4- cyclopropoxyphenyl) propyl)-6-methyl-2- (trifluoromethyl) thieno[2,3-d] pyrimidin-4-amine |
| 234 | 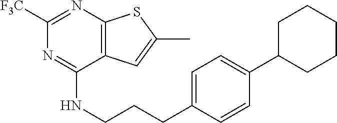 [SMILES: Cc1cc2c(NCCCc3ccc(C4CCCCC4)cc3)nc(C(F)(F)F)nc2s1] | KH 6080 | Method C 2 d (25° C.) | 18% | N-(3-(4- cyclohexylphenyl) propyl)-6-methyl-2- (trifluoromethyl) thieno[2,3-d] pyrimidin-4-amine |
| 235 | 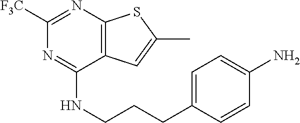 [SMILES: Cc1cc2c(NCCCc3ccc(N)cc3)nc(C(F)(F)F)nc2s1] | KH 6080 | Method C 2 d (25° C.) | 15% | N-(3-(4- aminophenyl)propyl)- 6-methyl-2- (trifluoromethyl) thieno[2,3-d]pyrimidin- 4-amine |
| 236 | 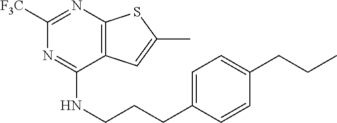 [SMILES: CCCc1ccc(CCCNc2nc(C(F)(F)F)nc3sc(C)cc23)cc1] | KH 6080 | Method C 2 d (25° C.) | 60% | 6-methyl-N-(3-(4- propylphenyl)propyl)- 2-(trifluoromethyl) thieno [2,3-d]pyrimidin- 4-amine |
| 237 | 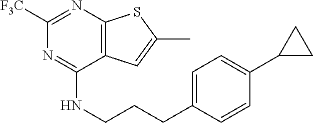 [SMILES: Cc1cc2c(NCCCc3ccc(C4CC4)cc3)nc(C(F)(F)F)nc2s1] | KH 6080 | Method C 16 h (25° C.) | 23% | N-(3-(4- cyclopropylphenyl) propyl)-6-methyl-2- (trifluoromethyl) thieno[2,3-d] pyrimidin-4-amine |
| 238 | 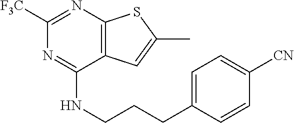 [SMILES: Cc1cc2c(NCCCc3ccc(C#N)cc3)nc(C(F)(F)F)nc2s1] | KH 6080 | Method C 2 d (25° C.) | 60% | 4-(3-((6-methyl-2- (trifluoromethyl) thieno[2,3-d] pyrimidin-4- yl)amino)propyl) benzonitrile |
| 239 | 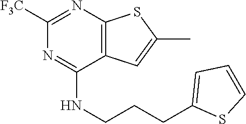 [SMILES: Cc1cc2c(NCCCc3cccs3)nc(C(F)(F)F)nc2s1] | KH 6080 | Method C 16 h (25° C.) | 10% | 6-methyl-N-(3- (thiophen-2- yl)propyl)-2- (trifluoromethyl) thieno[2,3-d] pyrimidin-4-amine |
| 240 | 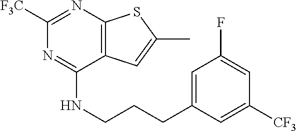 [SMILES: Cc1cc2c(NCCCc3cc(F)cc(C(F)(F)F)c3)nc(C(F)(F)F)nc2s1] | KH 6080 | Method C 16 h (25° C.) | 39% | N-(3-(3-fluoro-5- (trifluoromethyl) phenyl)propyl)-6- methyl-2- (trifluoromethyl) thieno[2,3-d] pyrimidin-4-amine |
| 241 | 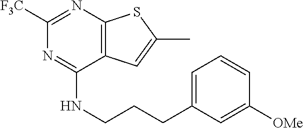 [SMILES: COc1cccc(CCCNc2nc(C(F)(F)F)nc3sc(C)cc23)c1] | KH 6080 | Method C 16 h (25° C.), 8 h (60° C.), 2 h (80° C.) | 32% | N-(3-(3- methoxyphenyl) propyl)-6-methyl-2- (trifluoromethyl) thieno[2,3-d] pyrimidin-4-amine |
| 242 | 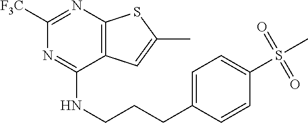 [SMILES: Cc1cc2c(NCCCc3ccc(S(C)(=O)=O)cc3)nc(C(F)(F)F)nc2s1] | KH 6080 | Method C 8 h (60° C.), 16 h (25° C.) | 15% | 6-methyl-N-(3-(4- (methylsulfonyl) phenyl)propyl)-2- (trifluoromethyl) thieno[2,3-d] pyrimidin-4-amine |
| 243 | 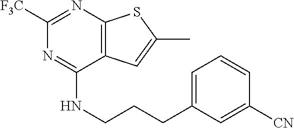 [SMILES: Cc1cc2c(NCCCc3cccc(C#N)c3)nc(C(F)(F)F)nc2s1] | KH 6080 | Method C 2 d (60° C.) | 23% | 3-(3-((6-methyl-2- (trifluoromethyl) thieno[2,3-d] pyrimidin-4- yl)amino)propyl) benzonitrile |
| 244 | 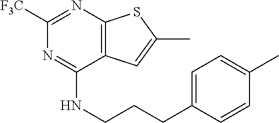 [SMILES: Cc1ccc(CCCNc2nc(C(F)(F)F)nc3sc(C)cc23)cc1] | KH 6080 | Method C 3 d (25° C.), 3 h (70° C.) | 45% | 6-methyl-N-(3-(p- tolyl)propyl)-2- (trifluoromethyl) thieno[2,3-d] pyrimidin-4-amine |
| 245 | 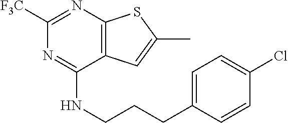 [SMILES: Cc1cc2c(NCCCc3ccc(Cl)cc3)nc(C(F)(F)F)nc2s1] | KH 6080 | Method C 3 d (25° C.) | 71% | N-(3-(4- chlorophenyl)propyl)- 6-methyl-2- (trifluoromethyl) thieno[2,3-d] pyrimidin-4-amine |
| 246 | 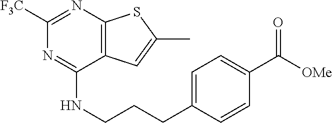 [SMILES: COC(=O)c1ccc(CCCNc2nc(C(F)(F)F)nc3sc(C)cc23)cc1] | KH 6080 | Method C 16 h (25° C.), 8 h (70° C.) | 50% | methyl 4-(3-((6- methyl-2- (trifluoromethyl) thieno[2,3-d] pyrimidin-4- yl)amino)propyl) benzoate |
| 247 | 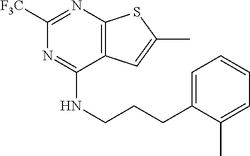 [SMILES: Cc1cc2c(NCCCc3ccccc3C)nc(C(F)(F)F)nc2s1] | KH 6080 | Method C 3 d (25° C.), 3 h (70° C.) | 22% | 6-methyl-N-(3-(o- tolyl)propyl)-2- (trifluoromethyl) thieno[2,3-d] pyrimidin-4-amine |
| 248 | 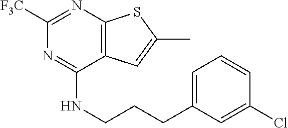 [SMILES: Cc1cc2c(NCCCc3cccc(Cl)c3)nc(C(F)(F)F)nc2s1] | KH 6080 | Method C 3 d (25° C.) 1 d (50° C.) | 64% | N-(3-(3- chlorophenyl)propyl)- 6-methyl-2- (trifluoromethyl) thieno[2,3-d] pyrimidin-4-amine |
| 249 | 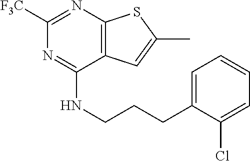 [SMILES: Cc1cc2c(NCCCc3ccccc3Cl)nc(C(F)(F)F)nc2s1] | KH 6080 | Method C 16 h (25° C.), 3 h (50° C.) | 68% | N-(3-(2- chlorophenyl)propyl)- 6-methyl-2- (trifluoromethyl)thieno [2,3-d]pyrimidin- 4-amine |
| 250 | 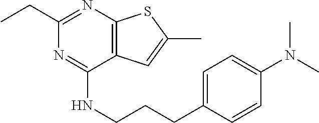 [SMILES: CCc1nc(NCCCc2ccc(N(C)C)cc2)c2cc(C)sc2n1] | KH 4007 | Method C 1 h (140° C.) microwave | 24% | N-(3-(4- (dimethylamino) phenyl)propyl)-2- ethyl-6-methylthieno [2,3-d]pyrimidin- 4-amine |
| 251 | 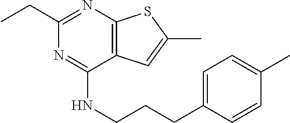 [SMILES: CCc1nc(NCCCc2ccc(C)cc2)c2cc(C)sc2n1] | KH 4007 | Method C 2 h (140° C.) microwave | 19% | 2-ethyl-6-methyl-N- (3-(p- tolyl)propyl)thieno [2,3-d]pyrimidin-4- amine |
| 252 | 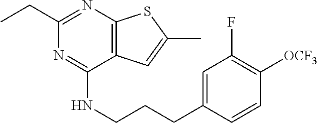 [SMILES: CCc1nc(NCCCc2ccc(OC(F)(F)F)c(F)c2)c2cc(C)sc2n1] | KH 4007 | Method C 4 h (120° C.) microwave | 8% | 2-ethyl-N-(3-(3- fluoro-4- (trifluoromethoxy) phenyl)propyl)-6- methylthieno[2,3- d]pyrimidin-4-amine |
| 253 | 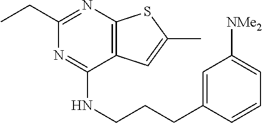 [SMILES: CCc1nc(NCCCc2cccc(N(C)C)c2)c2cc(C)sc2n1] | KH 4007 | Method C 2 h (140° C.) microwave | 13% | N-(3-(3- (dimethylamino) phenyl)propyl)- 2-ethyl- 6-methylthieno[2,3- d]pyrimidin-4-amine |
| 254 | 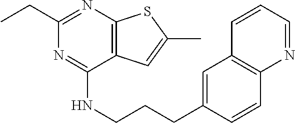 [SMILES: CCc1nc(NCCCc2ccc3ncccc3c2)c2cc(C)sc2n1] | KH 4007 | Method C 2 h (140° C.) microwave | 11% | 2-ethyl-6-methyl-N- (3-(quinolin-6- yl)propyl)thieno[2,3- d]pyrimidin-4-amine |
| 255 | 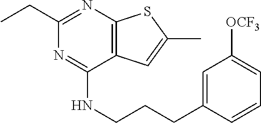 [SMILES: CCc1nc(NCCCc2cccc(OC(F)(F)F)c2)c2cc(C)sc2n1] | KH 4007 | Method C 1 h (140° C.), 1 h (160° C.) microwave | 32% | 2-ethyl-6-methyl-N- (3-(3- (trifluoromethoxy) phenyl)propyl)thieno [2,3-d]pyrimidin- 4-amine |
| 256 | 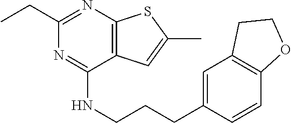 [SMILES: CCc1nc(NCCCc2ccc3c(c2)CCO3)c2cc(C)sc2n1] | KH 4007 | Method C 4 h (140° C.) microwave | 17% | N-(3-(2,3- dihydrobenzofuran- 5-yl)propyl)-2-ethyl- 6-methylthieno[2,3- d]pyrimidin-4-amine |
| 257 | 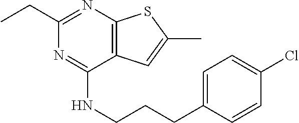 [SMILES: CCc1nc(NCCCc2ccc(Cl)cc2)c2cc(C)sc2n1] | KH 4007 | Method C 1 h (140° C.) 1 h (150° C.) microwave | 26% | N-(3-(4- chlorophenyl)propyl)- 2-ethyl-6- methylthieno[2,3- d]pyrimidin-4-amine |
| 258 | 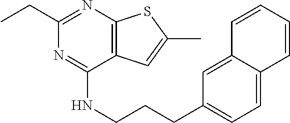 [SMILES: CCc1nc(NCCCc2ccc3ccccc3c2)c2cc(C)sc2n1] | KH 4007 | Method C 1 h (160° C.) 1 h (170° C.) microwave | 14% | 2-ethyl-6-methyl-N- (3-(naphthalen-2- yl)propyl)thieno[2,3- d]pyrimidin-4-amine |
| 259 | 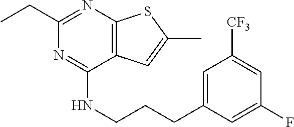 [SMILES: CCc1nc(NCCCc2cc(F)cc(C(F)(F)F)c2)c2cc(C)sc2n1] | KH 4007 | Method C 1 h (140° C.) 2 h (150° C.) microwave | 39% | 2-ethyl-N-(3-(3- fluoro-5- (trifluoromethyl) phenyl)propyl)-6- methylthieno[2,3- d]pyrimidin-4-amine |
| 260 | 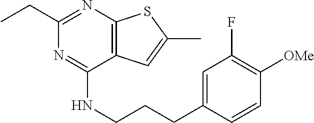 [SMILES: CCc1nc(NCCCc2ccc(OC)c(F)c2)c2cc(C)sc2n1] | KH 4007 | Method C 1 h (140° C.) 1 h (150° C.) microwave | 17% | 2-ethyl-N-(3-(3- fluoro-4- methoxyphenyl) propyl)-6- methylthieno[2,3- d]pyrimidin-4-amine |
| 261 | 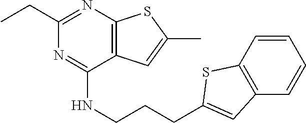 [SMILES: CCc1nc(NCCCc2cc3ccccc3s2)c2cc(C)sc2n1] | KH 4007 | Method C 1 h (150° C.) microwave | 20% | N-(3- (benzo[b]thiophen-2- yl)propyl)-2-ethyl-6- methylthieno[2,3- d]pyrimidin-4-amine |

Examples from Scheme 1 (Methods A-D)

| Cmpd. | |
|---|---|
| No. | Compound Data |
| 1 | ¹H NMR (400 MHz, CHLOROFORM-d) |
| | 7.34 (t, J = 7.63 Hz, 2H), 7.25 (d, J = 7.43 |
| | Hz, 2H), 7.18 (d, J = 7.43 Hz, 2H), 6.80 (s, |
| | 1H), 3.08 − 3.15 (m, J = 2.74 Hz, 1H), 2.86 |
| | (q, J = 7.43 Hz, 2H), 2.36 (s, 3H), 2.16 − |
| | 2.23 (m, 1H), 1.48 (q, J = 6.65 Hz, 1H), |
| | 1.39 − 1.46 (m, 1H), 1.35 (t, J = 7.43 Hz, |
| | 3H); LCMS: ESI [M + H]⁺ = 310.1 |
| 2 | ¹H NMR (400 MHz, CHLOROFORM-d) δ |
| | 7.34 (t, J = 7.43 Hz, 2H), 7.23 − 7.27 (m, J = |
| | 7.43 Hz, 1H), 7.18 (d, J = 7.43 Hz, 2H), |
| | 6.80 (s, 1H), 3.08 − 3.15 (m, J = 2.74 Hz, |
| | 1H), 2.86 (q, J = 7.43 Hz, 2H), 2.37 (s, |
| | 3H), 2.17 − 2.23 (m, 1H), 1.48 (q, J = 6.26 |
| | Hz, 1H), 1.41 (td, J = 5.04, 9.88 Hz, 1H), |
| | 1.35 (t, J = 7.63 Hz, 3H); LCMS: ESI |
| | [M + H]⁺ = 310.1 |
| 3 | ¹H NMR (400 MHz, CHLOROFORM-d) δ |
| | 7.53 − 7.58 (m, J = 8.22 Hz, 2H), 7.32 − |
| | 7.37 (m, J = 7.83 Hz, 2H), 6.66 (br. s., 1H), |
| | 3.69 (q, J = 6.65 Hz, 2H), 2.85 (quin, J = |
| | 7.14 Hz, 4H), 2.53 (s, 3H), 2.07 (quin, J = |
| | 7.14 Hz, 2H), 1.36 (t, J = 7.43 Hz, 3H); |
| | LCMS: ESI [M + H]⁺ = 380.1 |
| 4 | ¹H NMR (400 MHz, CHLOROFORM-d) δ |
| | 7.07 − 7.16 (m, 2H), 6.97 (d, J = 8.22 Hz, |
| | 1H), 6.80 (s, 1H), 3.06 (tdd, J = 2.54, 4.84, |
| | 7.29 Hz, 1H), 2.89 (q, J = 7.70 Hz, 2H), |
| | 2.46 (s, 3H), 2.13 − 2.20 (m, 1H), 1.39 − |
| | 1.45 (m, 2H), 1.36 (t, J = 7.63 Hz, 3H); |
| | LCMS: ESI [M + H]⁺ = 346.1 |
| 5 | ¹H NMR (400 MHz, CHLOROFORM-d) δ |
| | 7.31 − 7.40 (m, 4H), 7.24 (t, J = 7.04 Hz, |
| | 1H), 6.80 (s, 1H), 4.82 (qd, J = 6.68, 13.21 |
| | Hz, 1H), 3.67 − 3.77 (m, J = 5.87, 5.87 Hz, |
| | 1H), 2.84 (q, J = 7.43 Hz, 2H), 2.68 − 2.77 |
| | (m, 2H), 2.47 − 2.58 (m, 5H), 1.35 (t, J = |
| | 7.63 Hz, 3H); LCMS: ESI [M + H]⁺ = 324.2 |
| 6 | ¹H NMR (400 MHz, CHLOROFORM-d) δ |
| | 7.32 − 7.38 (m, 2H), 7.28 (d, J = 5.09 Hz, |
| | 1H), 7.15 (d, J = 7.43 Hz, 2H), 6.84 (s, |
| | 1H), 3.12 (br. s., 1H), 2.90 (q, J = 7.43 Hz, |
| | 2H), 2.34 (s, 3H), 2.24 (t, J = 6.46 Hz, 1H), |
| | 1.52 (t, J = 6.65 Hz, 2H), 1.38 (t, J = 7.63 |
| | Hz, 3H); LCMS: ESI [M + H]⁺ = 310.1 |
| 7 | ¹H NMR (400 MHz, CHLOROFORM-d) δ |
| | 7.29 (d, J = 8.22 Hz, 2H), 7.17 (d, J = 8.22 |
| | Hz, 2H), 6.66 (s, 1H), 3.86 (q, J = 6.78 Hz, |
| | 2H), 2.97 (t, J = 7.04 Hz, 2H), 2.87 (q, J = |
| | 7.43 Hz, 2H), 2.52 (s, 3H), 1.38 (t, J = 7.63 |
| | Hz, 3H); LCMS: ESI [M + H]⁺ = 332.1 |
| 8 | ¹H NMR (400 MHz, CHLOROFORM-d) δ |
| | 7.22 − 7.27 (m, 3H), 7.13 (d, J = 7.04 Hz, |
| | 1H), 6.69 (br. s., 1H), 3.89 (q, J = 6.52 Hz, |
| | 2H), 2.99 (t, J = 7.04 Hz, 2H), 2.89 (q, J = |
| | 7.83 Hz, 2H), 2.54 (s, 3H), 1.39 (t, J = 7.43 |
| | Hz, 3H); LCMS: ESI [M + H]⁺ = 332.1 |
| 9 | ¹H NMR (400 MHz, CHLOROFORM-d) δ |
| | 7.36 − 7.40 (m, 1H), 7.23 − 7.27 (m, J = |
| | 3.13 Hz, 1H), 7.16 − 7.23 (m, 2H), 6.70 (s, |
| | 1H), 3.91 (q, J = 6.78 Hz, 2H), 3.15 (t, J = |
| | 7.04 Hz, 2H), 2.87 (q, J = 7.56 Hz, 2H), |
| | 2.53 (s, 3H), 1.38 (t, J = 7.63 Hz, 3H); |
| | LCMS: ESI [M + H]⁺ = 332.1 |
| 10 | ¹H NMR (400 MHz, CHLOROFORM-d) δ |
| | 7.77 − 7.86 (m, 3H), 7.69 (s, 1H), 7.44 − |
| | 7.52 (m, 2H), 7.40 (d, J = 8.61 Hz, 1H), |
| | 6.64 (br. s., 1H), 3.99 (q, J = 6.39 Hz, 2H), |
| | 3.17 (t, J = 7.04 Hz, 2H), 2.90 (q, J = 7.56 |
| | Hz, 2H), 2.49 (s, 3H), 1.41 (dt, J = 0.98, |
| | 7.53 Hz, 3H LCMS: ESI [M + H]⁺ = 348.2 |
| 11 | ¹H NMR (400 MHz, CHLOROFORM-d) δ |
| | 7.28 − 7.35 (m, 2H), 6.93 − 7.01 (m, 3H), |
| | 6.78 (s, 1H), 4.19 − 4.26 (m, 2H), 4.06 (q, J = |
| | 5.09 Hz, 2H), 2.87 (q, J = 7.56 Hz, 2H), |
| | 2.54 (s, 3H), 1.37 (t, J = 7.63 Hz, 3H); |
| | LCMS: ESI [M + H]⁺ = 314.1 |
| 12 | ¹H NMR (400 MHz, CHLOROFORM-d) δ |
| | 8.52 (d, J = 4.70 Hz, 2H), 7.20 (d, J = 5.48 |
| | Hz, 2H), 6.75 (s, 1H), 3.92 (q, J = 6.52 Hz, |
| | 2H), 3.04 (t, J = 7.04 Hz, 2H), 2.89 (q, J = |
| | 7.56 Hz, 2H), 2.51 (s, 3H), 1.39 (t, J = 7.63 |
| | Hz, 3H); LCMS: m/z calcd. for |
| | C₁₈H₁₈F₃N₃S [M + H]⁺: 366.12 LCMS: ESI |
| | [M + H]⁺ = 299.1 |
| 13 | ¹H NMR (400 MHz, CHLOROFORM-d) δ |
| | 8.46 − 8.53 (m, 2H), 7.59 (d, J = 7.83 Hz, |
| | 1H), 7.23 − 7.27 (m, 1H), 6.73 (s, 1H), 3.90 |
| | (q, J = 6.65 Hz, 2H), 3.03 (t, J = 7.04 Hz, |
| | 2H), 2.88 (q, J = 7.56 Hz, 2H), 2.51 (s, |
| | 3H), 1.38 (t, J = 7.63 Hz, 3H); LCMS: ESI |
| | [M + H]⁺ = 299.1 |
| 14 | ¹H NMR (400 MHz, CHLOROFORM-d) δ |
| | 8.59 (d, J = 5.09 Hz, 1H), 7.68 (t, J = 7.63 |
| | Hz, 1H), 7.19 − 7.26 (m, 2H), 6.85 (br. s., |
| | 1H), 4.05 (q, J = 5.74 Hz, 2H), 3.20 (t, J = |
| | 6.06 Hz, 2H), 2.88 (q, J = 7.70 Hz, 2H), |
| | 2.55 (s, 3H), 1.37 (t, J = 7.43 Hz, 3H); |
| | LCMS: ESI [M + H]⁺ = 299.1 |
| 15 | ¹H NMR (400 MHz, CHLOROFORM-d) δ |
| | 7.14 − 7.19 (m, J = 8.61 Hz, 2H), 6.85 − |
| | 6.90 (m, J = 8.22 Hz, 2H), 6.65 (s, 1H), |
| | 3.83 − 3.88 (m, 2H), 3.81 (s, 3H), 2.93 (t, J = |
| | 7.04 Hz, 2H), 2.87 (q, J = 7.43 Hz, 2H), |
| | 2.52 (s, 3H), 1.38 (t, J = 7.63 Hz, 3H); |
| | LCMS: ESI [M + H]⁺ = 328.2 |
| 16 | ¹H NMR (400 MHz, CHLOROFORM-d) δ |
| | 8.17 (d, J = 8.22 Hz, 2H), 7.41 (d, J = 8.22 |
| | Hz, 2H), 6.72 (s, 1H), 3.93 (q, J = 6.65 Hz, |
| | 2H), 3.15 (t, J = 7.04 Hz, 2H), 2.89 (q, J = |
| | 7.70 Hz, 2H), 2.52 (s, 3H), 1.39 (t, J = 7.63 |
| | Hz, 3H); LCMS: ESI [M + H]⁺ = 343.1 |
| 17 | (s, 3H), 2.81 (q, J = 7.43 Hz, 2H), 2.72 (t, J |
| | ¹H NMR (400 MHz, CHLOROFORM-d) δ |
| | 7.28 − 7.34 (m, 2H), 7.21 (d, J = 7.43 Hz, |
| | 3H), 6.83 (s, 1H), 3.73 − 3.80 (m, 2H), 3.34 |
| | 7.43 Hz, 2H), 2.47 (s, 3H), 2.05 (quin, J = |
| | 7.43 Hz, 2H), 1.33 (t, J = 7.63 Hz, 3H); |
| | LCMS: ESI [M + H]⁺ = 326.2 |
| 18 | ¹H NMR (400 MHz, CHLOROFORM-d) δ |
| | 7.11 − 7.17 (m, J = 8.22 Hz, 2H), 6.82 − |
| | 6.88 (m, J = 8.22 Hz, 2H), 6.57 (s, 1H), |
| | 3.80 (s, 3H), 3.66 (q, J = 6.52 Hz, 2H), 2.84 |
| | (q, J = 7.43 Hz, 2H), 2.71 (t, J = 7.43 Hz, |
| | 2H), 2.52 (s, 3H), 2.00 (quin, J = 7.04 Hz, |
| | 2H), 1.35 (t, J = 7.63 Hz, 3H); LCMS: ESI |
| | [M + H]⁺ = 342.2 |
| 19 | ¹H NMR (400 MHz, CHLOROFORM-d) δ |
| | 7.55 − 7.60 (m, J = 7.83 Hz, 2H), 7.34 − |
| | 7.40 (m, J = 7.83 Hz, 2H), 6.70 (s, 1H), |
| | 3.90 (q, J = 6.65 Hz, 2H), 3.08 (t, J = 7.04 |
| | Hz, 2H), 2.88 (q, J = 7.70 Hz, 2H), 2.52 (s, |
| | 3H), 1.39 (t, J = 7.43 Hz, 3H); LCMS: ESI |
| | [M + H]⁺ = 366.1 |
| 20 | ¹H NMR (400 MHz, CHLOROFORM-d) δ |
| | 7.52 (s, 2H), 7.41 − 7.47 (m, 2H), 6.71 (br. |
| | s., 1H), 3.91 (q, J = 6.91 Hz, 2H), 3.08 (t, J = |
| | 7.04 Hz, 2H), 2.89 (q, J = 7.56 Hz, 2H), |
| | 2.53 (s, 3H), 1.39 (t, J = 7.63 Hz, 3H); |
| | LCMS: ESI [M + H]⁺ = 366.1 |
| 21 | ¹H NMR (400 MHz, CHLOROFORM-d) δ |
| | 7.67 (d, J = 7.83 Hz, 1H), 7.50 (t, J = 7.43 |
| | Hz, 1H), 7.43 (d, J = 7.83 Hz, 1H), 7.31 − |
| | 7.38 (m, 1H), 6.66 (s, 1H), 5.09 (br. s., 1H), |
| | 3.89 (q, J = 6.65 Hz, 2H), 3.19 (t, J = 7.04 |
| | Hz, 2H), 2.88 (q, J = 7.56 Hz, 2H), 2.54 (s, |
| | 3H), 1.39 (t, J = 7.63 Hz, 3H); LCMS: ESI |
| | [M + H]⁺ = 366.1 |
| 22 | ¹H NMR (400 MHz, CHLOROFORM-d) δ |
| | 6.72 − 6.82 (m, 3H), 6.55 (s, 1H), 3.86 (d, J = |
| | 10.17 Hz, 6H), 3.67 (q, J = 6.65 Hz, 2H), |
| | 2.83 (q, J = 7.43 Hz, 2H), 2.71 (t, J = 7.24 |
| | Hz, 2H), 2.52 (s, 3H), 2.01 (quin, J = 7.14 |
| | Hz, 2H), 1.35 (t, J = 7.63 Hz, 3H); LCMS: |
| | ESI [M + H]⁺ = 372.2 |
| 23 | ¹H NMR (400 MHz, CHLOROFORM-d) δ |
| | 7.27 − 7.31 (m, 2H), 7.20 (d, J = 6.65 Hz, |
| | 3H), 6.60 (s, 1H), 2.83 (q, J = 7.43 Hz, |
| | 2H), 2.72 − 2.79 (m, 2H), 2.53 (s, 3H), 1.86 − |
| | 2.04 (m, 2H), 1.29 − 1.39 (m, 6H); LCMS: |
| | ESI [M + H]⁺ = 326.1 |
| 24 | ¹H NMR (400 MHz, CHLOROFORM-d) δ |
| | 7.33 (s, 1H), 6.67 (s, 1H), 6.31 (br. s., 1H), |
| | 6.06 (d, J = 2.35 Hz, 1H), 3.68 (q, J = 6.65 |
| | Hz, 2H), 2.84 (q, J = 7.43 Hz, 2H), 2.77 (t, |
| | J = 7.24 Hz, 2H), 2.53 (s, 3H), 2.04 (quin, J = |
| | 7.14 Hz, 2H), 1.35 (t, J = 7.63 Hz, 3H); |
| | LCMS: ESI [M + H]⁺ = 302.1 |
| 25 | ¹H NMR (400 MHz, CHLOROFORM-d) δ |
| | 7.28 − 7.32 (m, 2H), 7.17 − 7.23 (m, 3H), |
| | 6.70 (s, 1H), 3.65 (q, J = 6.39 Hz, 2H), 2.84 |
| | (q, J = 7.83 Hz, 2H), 2.70 (t, J = 7.04 Hz, |
| | 2H), 2.54 (s, 3H), 1.67 − 1.81 (m, 4H), 1.35 |
| | (t, J = 7.63 Hz, 3H); LCMS: ESI [M + H]⁺ = |
| | 326.2 |
| 26 | ¹H NMR (400 MHz, CHLOROFORM-d) δ |
| | 6.73 (s, 2H), 6.58 (s, 1H), 6.22 (s, 2H), 4.68 |
| | (br. s., 1H), 4.05 (t, J = 6.46 Hz, 2H), 3.67 |
| | (q, J = 6.00 Hz, 2H), 2.83 (q, J = 7.43 Hz, |
| | 2H), 2.53 (s, 3H), 2.15 (quin, J = 6.46 Hz, |
| | 2H), 1.35 (t, J = 7.63 Hz, 3H); LCMS: ESI |
| | [M + H]⁺ = 374.1 |
| 27 | ¹H NMR (400 MHz, CHLOROFORM-d) δ |
| | 6.73 (s, 2H), 6.58 (s, 1H), 6.22 (s, 2H), 4.68 |
| | (br. s., 1H), 4.05 (t, J = 6.46 Hz, 2H), 3.67 |
| | (q, J = 6.00 Hz, 2H), 2.83 (q, J = 7.43 Hz, |
| | 2H), 2.53 (s, 3H), 2.15 (quin, J = 6.46 Hz, |
| | 2H), 1.35 (t, J = 7.63 Hz, 3H); LCMS: ESI |
| | [M + H]⁺ = 301.1 |
| 28 | ¹H NMR (400 MHz, CHLOROFORM-d) δ |
| | 7.18 − 7.25 (m, J = 9.39 Hz, 1H), 7.08 − |
| | 7.16 (m, 2H), 6.85 (s, 1H), 5.62 (br. s., 1H), |
| | 3.04 (br. s., 1H), 2.57 (s, 3H), 2.14 − 2.21 |
| | (m, J = 6.26 Hz, 1H), 1.41 (q, J = 6.65 Hz, |
| | 1H), 1.30 − 1.37 (m, J = 5.87 Hz, 1H); |
| | LCMS: ESI [M + H]⁺ = 386.1 |
| 29 | ¹H NMR (400 MHz, CHLOROFORM-d) ¿ |
| | 8.84 (d, J = 4.30 Hz, 1H), 8.46 (d, J = 7.83 |
| | Hz, 1H), 7.83 (dt, J = 1.96, 7.63 Hz, 1H), |
| | 7.35 − 7.42 (m, 1H), 7.30 (d, J = 5.87 Hz, |
| | 1H), 7.15 (d, J = 8.61 Hz, 2H), 7.06 (d, J = |
| | 5.87 Hz, 1H), 6.85 (d, J = 8.22 Hz, 2H), |
| | 3.76 − 3.88 (m, 5H), 2.76 (t, J = 7.04 Hz, |
| | 2H), 2.04 − 2.14 (m, 2H); LCMS: ESI |
| | [M + H]⁺ = 377.1 |
| 30 | ¹H NMR (400 MHz, CHLOROFORM-d) δ |
| | 8.84 (d, J = 3.91 Hz, 1H), 8.44 (d, J = 7.83 |
| | Hz, 1H), 7.82 (t, J = 7.24 Hz, 1H), 7.54 (d, |
| | J = 7.83 Hz, 2H), 7.35 − 7.41 (m, 1H), 7.29 − |
| | 7.35 (m, 3H), 7.12 (d, J = 5.87 Hz, 1H), |
| | 5.45 (br. s., 1H), 3.83 (q, J = 6.39 Hz, 2H), |
| | 2.85 (t, J = 7.24 Hz, 2H), 2.13 (quin, J = |
| | 7.24 Hz, 2H); LCMS: ESI [M + H]⁺ = 415.1 |
| 31 | ¹H NMR (400 MHz, CHLOROFORM-d) δ |
| | 8.82 (d, J = 4.70 Hz, 1H), 8.39 (d, J = 7.83 |
| | Hz, 1H), 7.79 (t, J = 7.43 Hz, 1H), 7.41 (d, |
| | J = 7.04 Hz, 1H), 7.43 − 7.48 (m, J = 7.04 |
| | Hz, 1H), 7.37 (d, J = 5.87 Hz, 1H), 7.09 − |
| | 7.19 (m, 2H), 6.96 − 7.02 (m, J = 8.22 Hz, |
| | 1H), 3.18 (br. s., 1H), 2.23 − 2.30 (m, J = |
| | 9.00 Hz, 1H), 1.52 − 1.59 (m, J = 5.09 Hz, |
| | 1H), 1.48 (q, J = 6.65 Hz, 1H); LCMS: ESI |
| | [M + H]⁺ = 381.0 |
| 32 | ¹H NMR (400 MHz, CHLOROFORM-d) δ |
| | 8.82 (br. s., 1H), 8.29 (d, J = 7.83 Hz, 1H), |
| | 7.71 (t, J = 7.24 Hz, 1H), 7.33 − 7.40 (m, J = |
| | 5.09 Hz, 1H), 7.06 − 7.16 (m, 3H), 6.90 − |
| | 6.98 (m, J = 7.83 Hz, 1H), 5.94 (br. s., 1H), |
| | 3.10 (br. s., 1H), 2.11 − 2.19 (m, 1H), 1.41 |
| | (t, J = 6.85 Hz, 2H); LCMS: ESI [M + H]⁺ = |
| | 415.0 |
| 33 | ¹H NMR (400 MHz, CHLOROFORM-d) δ |
| | 8.81 (d, J = 4.30 Hz, 1H), 8.34 (d, J = 7.83 |
| | Hz, 1H), 7.73 (t, J = 7.63 Hz, 1H), 7.32 − |
| | 7.39 (m, 1H), 7.07 − 7.19 (m, 2H), 6.90 − |
| | 7.01 (m, 2H), 3.12 (br. s., 1H), 2.53 (s, 3H), |
| | 2.15 − 2.23 (m, J = 5.87 Hz, 1H), 1.41 − |
| | 1.50 (m, 2H); LCMS: ESI [M + H]⁺ = 395.1 |
| 34 | ¹H NMR (400 MHz, CHLOROFORM-d) δ |
| | 7.20 − 7.26 (m, J = 8.61 Hz, 2H), 7.12 − |
| | 7.17 (m, J = 7.83 Hz, 2H), 6.64 (s, 1H), |
| | 3.67 (q, J = 6.91 Hz, 2H), 2.84 (q, J = 7.69 |
| | Hz, 2H), 2.76 (t, J = 7.43 Hz, 2H), 2.53 (s, |
| | 3H), 2.02 (quin, J = 7.24 Hz, 2H), 1.35 (t, J = |
| | 7.43 Hz, 3H); LCMS: ESI [M + H]⁺ = |
| | 396.1 |
| 35 | ¹H NMR (400 MHz, CHLOROFORM-d) δ |
| | 8.85 (d, J = 4.30 Hz, 1H), 8.46 (d, J = 7.83 |
| | Hz, 1H), 7.83 (t, J = 7.83 Hz, 1H), 7.35 − |
| | 7.41 (m, 1H), 7.32 (d, J = 5.87 Hz, 1H), |
| | 7.24 (d, J = 8.22 Hz, 2H), 7.14 (d, J = 6.65 |
| | Hz, 3H), 5.49 (br. s., 1H), 3.83 (q, J = 6.52 |
| | Hz, 2H), 2.80 (t, J = 7.43 Hz, 2H), 2.10 |
| | (quin, J = 7.24 Hz, 2H); LCMS: ESI |
| | [M + H]⁺ = 431.0 |
| 36 | ¹H NMR (400 MHz, CHLOROFORM-d) δ |
| | 7.25 (d, J = 8.22 Hz, 2H), 7.15 (d, J = 8.22 |
| | Hz, 2H), 6.71 (s, 1H), 5.20 (br. s., 1H), 3.67 − |
| | 3.76 (m, 2H), 2.77 (t, J = 7.43 Hz, 2H), |
| | 2.60 (s, 3H), 2.05 (quin, J = 7.04 Hz, 2H); |
| | LCMS: ESI [M + H]⁺ = 436.0 |
| 37 | ¹H NMR (400 MHz, CHLOROFORM-d) δ |
| | 7.22 − 7.27 (m, 1H), 6.77 − 6.86 (m, 3H), |
| | 6.62 (s, 1H), 3.88 (q, J = 6.39 Hz, 2H), 3.80 |
| | (s, 3H), 2.97 (t, J = 7.04 Hz, 2H), 2.87 (q, J = |
| | 7.43 Hz, 2H), 2.52 (s, 3H), 1.39 (t, J = |
| | 7.63 Hz, 3H); LCMS: ESI [M + H]⁺ = 328.1 |
| 38 | ¹H NMR (400 MHz, CHLOROFORM-d) δ |
| | 9.42 (s, 1H), 8.79 (d, J = 5.09 Hz, 1H), 8.22 |
| | (d, J = 4.30 Hz, 1H), 7.08 − 7.21 (m, 4H), |
| | 6.94 − 7.00 (m, J = 6.26 Hz, 2H), 6.92 (br. |
| | s., 1H), 3.08 − 3.16 (m, J = 5.87 Hz, 1H), |
| | 2.55 (s, 3H), 2.18 (t, J = 7.43 Hz, 1H), 1.45 |
| | (t, J = 6.85 Hz, 2H); LCMS: ESI [M + H]⁺ = |
| | 396.1 |
| 39 | ¹H NMR (400 MHz, CHLOROFORM-d) δ |
| | 8.15 (s, 1H), 8.11 (d, J = 7.83 Hz, 1H), 7.59 |
| | (d, J = 7.83 Hz, 1H), 7.49 (t, J = 7.83 Hz, |
| | 1H), 6.69 (s, 1H), 3.93 (q, J = 6.65 Hz, |
| | 2H), 3.14 (t, J = 7.04 Hz, 2H), 2.88 (q, J = |
| | 7.43 Hz, 2H), 2.53 (s, 3H), 1.39 (t, J = 7.43 |
| | Hz, 3H); LCMS: ESI [M + H]⁺ = 343.1 |
| 40 | ¹H NMR (400 MHz, CHLOROFORM-d) δ |
| | 6.90 (d, J = 9.00 Hz, 2H), 6.84 (d, J = 9.00 |
| | Hz, 2H), 6.76 (s, 1H), 4.14 − 4.20 (m, 2H), |
| | 4.03 (q, J = 5.48 Hz, 2H), 3.78 (s, 3H), 2.86 |
| | (q, J = 7.70 Hz, 2H), 2.54 (s, 3H), 1.36 (t, J = |
| | 7.63 Hz, 3H); LCMS: ESI [M + H]⁺ = 344.1 |
| 41 | ¹H NMR (400 MHz, CHLOROFORM-d) δ |
| | 9.03 (d, J = 4.70 Hz, 2H), 7.39 − 7.58 (m, J = |
| | 4.70, 4.70 Hz, 2H), 7.07 − 7.16 (m, 1H), |
| | 6.94 − 7.04 (m, J = 5.09 Hz, 2H), 3.15 (br. |
| | s., 1H), 2.49 (s, 3H), 2.21 − 2.30 (m, J = |
| | 5.87 Hz, 1H), 1.41 − 1.50 (m, J = 5.87 Hz, |
| | 2H); LCMS: ESI [M + H]⁺ = 396.1 |
| 42 | ¹H NMR (400 MHz, CHLOROFORM-d) δ |
| | 7.99 (s, 1H), 7.45 (s, 1H), 7.16 − 7.23 (m, J = |
| | 9.00 Hz, 1H), 7.07 − 7.16 (m, 2H), 7.00 |
| | (br. s., 1H), 6.88 (s, 1H), 3.15 (br. s., 1H), |
| | 2.51 (s, 3H), 2.14 − 2.22 (m, J = 7.04 Hz, |
| | 1H), 1.37 − 1.48 (m, J = 5.87 Hz, 2H); |
| | LCMS: ESI [M + H]⁺ = 401.0 |
| 43 | ¹H NMR (400 MHz, CHLOROFORM-d) δ |
| | 7.08 − 7.16 (m, 1H), 7.00 − 7.07 (m, 1H), |
| | 6.91 − 6.96 (m, J = 7.83 Hz, 1H), 6.72 (s, |
| | 1H), 3.00 − 3.06 (m, J = 2.74 Hz, 1H), 2.46 |
| | (s, 3H), 2.06 − 2.16 (m, 2H), 1.31 − 1.42 (m, |
| | 2H), 1.01 − 1.11 (m, J = 9.00 Hz, 2H), 0.96 |
| | (d, J = 8.22 Hz, 2H); LCMS: ESI [M + H]⁺ = |
| | 358.1 |
| 44 | ¹H NMR (400 MHz, CHLOROFORM-d) δ |
| | 9.56 − 9.60 (m, J = 2.35 Hz, 1H), 8.64 (dd, |
| | J = 1.57, 4.70 Hz, 1H), 8.51 − 8.57 (m, 1H), |
| | 7.27 − 7.32 (m, 1H), 7.10 − 7.18 (m, 1H), |
| | 6.93 − 7.06 (m, 2H), 6.81 (s, 1H), 5.53 (br. |
| | s., 1H), 3.12 − 3.18 (m, 1H), 2.56 (s, 3H), |
| | 2.12 − 2.19 (m, 1H), 1.39 − 1.45 (m, 2H); |
| | LCMS: ESI [M + H]⁺ = 395.0 |
| 45 | ¹H NMR (400 MHz, CHLOROFORM-d) δ |
| | 8.90 (d, J = 4.70 Hz, 1H), 8.41 (d, J = 7.83 |
| | Hz, 1H), 7.85 − 7.92 (m, J = 5.48 Hz, 1H), |
| | 7.72 (br. s., 1H), 7.51 (br. s., 1H), 7.09 − |
| | 7.18 (m, 2H), 6.96 (br. s., 1H), 3.24 − 3.39 |
| | (m, 1H), 2.16 − 2.22 (m, J = 7.04 Hz, 1H), |
| | 1.53 − 1.60 (m, J = 5.09 Hz, 1H), 1.47 (q, |
| | J = 6.91 Hz, 1H); LCMS: ESI [M + H]⁺ = |
| | 449.0 |
| 46 | ¹H NMR (400 MHz, CHLOROFORM-d) δ |
| | 8.64 (d, J = 2.74 Hz, 1H), 8.36 (dd, J = |
| | 4.70, 9.00 Hz, 1H), 7.37 − 7.44 (m, 1H), |
| | 7.09 − 7.19 (m, 2H), 6.89 − 6.99 (m, 2H), |
| | 3.11 (br. s., 1H), 2.53 (s, 3H), 2.19 (br. s., |
| | 1H), 1.41 − 1.52 (m, J = 6.85, 6.85 Hz, 2H); |
| | LCMS: ESI [M + H]⁺ = 413.0 |
| 47 | ¹H NMR (400 MHz, CHLOROFORM-d) o |
| | 8.64 (d, J = 6.26 Hz, 2H), 8.25 (d, J = 6.26 |
| | Hz, 2H), 7.10 − 7.19 (m, 1H), 7.01 − 7.08 |
| | (m, 1H), 6.94 − 7.00 (m, J = 8.61 Hz, 1H), |
| | 6.86 (s, 1H), 5.63 (br. s., 1H), 3.13 (ddd, |
| | J = 2.54, 5.97, 8.71 Hz, 1H), 2.59 (s, 3H), |
| | 2.15 (dt, J = 3.13, 7.83 Hz, 1H), 1.41 − 1.48 |
| | (m, 2H); LCMS: ESI [M + H]⁺ = 395.1 |
| 48 | ¹H NMR (400 MHz, CHLOROFORM-d) δ |
| | 7.53 − 7.57 (m, J = 8.22 Hz, 2H), 7.32 − |
| | 7.37 (m, J = 7.83 Hz, 2H), 6.67 (s, 1H), |
| | 5.21 (br. s., 1H), 3.72 (q, J = 6.39 Hz, 2H), |
| | 2.82 (t, J = 7.24 Hz, 2H), 2.58 (s, 3H), 2.08 |
| | (quin, J = 7.24 Hz, 2H); LCMS: ESI |
| | [M + H]⁺ = 420.1 |
| 49 | ¹H NMR (400 MHz, CHLOROFORM-d) δ |
| | 7.11 − 7.17 (m, J = 8.61 Hz, 2H), 6.83 − |
| | 6.88 (m, J = 8.61 Hz, 2H), 6.59 (s, 1H), |
| | 5.15 (br. s., 1H), 3.80 (s, 3H), 3.69 (q, J = |
| | 6.52 Hz, 2H), 2.72 (t, J = 7.24 Hz, 2H), |
| | 2.57 (s, 3H), 2.02 (quin, J = 7.04 Hz, 2H); |
| | LCMS: ESI [M + H]⁺ = 382.1 |
| 50 | ¹H NMR (400 MHz, CHLOROFORM-d) δ |
| | 7.02 − 7.19 (m, 3H), 6.83 (s, 1H), 5.75 (br. |
| | s., 1H), 3.02 − 3.07 (m, J = 3.13 Hz, 1H), |
| | 2.53 (s, 3H), 2.11 − 2.18 (m, J = 6.26, 6.26 |
| | Hz, 1H), 2.00 (t, J = 18.59 Hz, 3H), 1.32 − |
| | 1.42 (m, 2H); LCMS: ESI [M + H]⁺ = 382.0 |
| 51 | ¹H NMR (400 MHz, CHLOROFORM-d) o |
| | 7.28 − 7.33 (m, 2H), 7.19 − 7.24 (m, 3H), |
| | 6.61 (br. s., 1H), 3.62 − 3.70 (m, 2H), 2.75 |
| | (t, J = 7.43 Hz, 2H), 2.53 (s, 3H), 2.04 (td, |
| | J = 6.80, 14.18 Hz, 2H), 1.40 (s, 9H); |
| | LCMS: ESI [M + H]⁺ = 340.2 |
| 52 | ¹H NMR (400 MHz, CHLOROFORM-d) δ |
| | 7.08 − 7.17 (m, 2H), 6.92 − 6.98 (m, J = |
| | 3.91 Hz, 1H), 6.84 (br. s., 1H), 3.06 (br. s., |
| | 1H), 2.81 − 2.88 (m, 2H), 2.45 (s, 3H), 2.20 |
| | (br. s., 1H), 1.82 − 1.92 (m, 2H), 1.39 − 1.50 |
| | (m, 2H), 1.01 (t, J = 7.43 Hz, 3H); LCMS: |
| | ESI [M + H]⁺ = 360.1 |
| 53 | ¹H NMR (400 MHz, CHLOROFORM-d) δ |
| | 7.11 − 7.19 (m, 2H), 6.98 − 7.04 (m, J = |
| | 7.43 Hz, 1H), 6.97 (s, 1H), 3.01 − 3.07 (m, |
| | 1H), 2.49 (s, 3H), 2.28 (s, 3H), 1.98 − 2.05 |
| | (m, 1H), 1.39 − 1.46 (m, 1H), 1.32 (q, J = |
| | 6.65 Hz, 1H); LCMS: ESI [M + H]⁺ = 364.0 |
| 54 | ¹H NMR (400 MHz, CHLOROFORM-d) δ |
| | 7.28 − 7.34 (m, 2H), 7.20 − 7.25 (m, 3H), |
| | 6.60 (s, 1H), 3.62 − 3.70 (m, 2H), 2.71 − |
| | 2.83 (m, 4H), 2.52 (s, 3H), 2.04 (quin, J = |
| | 7.14 Hz, 2H), 1.85 (qd, J = 7.43, 14.87 Hz, |
| | 2H), 1.00 (t, J = 7.24 Hz, 3H) LCMS: ESI |
| | [M + H]⁺ = 326.2 |
| 55 | ¹H NMR (400 MHz, CHLOROFORM-d) δ |
| | 7.27 − 7.34 (m, 2H), 7.17 − 7.24 (m, 3H), |
| | 6.58 (s, 1H), 3.61 − 3.68 (m, 2H), 2.69 − |
| | 2.78 (m, 2H), 2.55 (s, 3H), 2.49 (s, 3H), |
| | 1.97 − 2.06 (m, 2H); LCMS: ESI [M + H]⁺ = |
| | 330.1 |
| 56 | ¹H NMR (400 MHz, CHLOROFORM-d) o |
| | 8.31 − 8.37 (m, 2H), 7.10 − 7.18 (m, 1H), |
| | 7.05 (t, J = 8.61 Hz, 3H), 6.93 − 6.99 (m, |
| | J = 8.61 Hz, 1H), 6.79 (s, 1H), 3.08 − 3.15 |
| | (m, J = 2.35 Hz, 1H), 2.54 (s, 3H), 2.12 − |
| | 2.19 (m, J = 5.09 Hz, 1H), 1.43 (t, J = 6.85 |
| | Hz, 2H); LCMS: ESI [M + H]⁺ = 412.1 |
| 57 | ¹H NMR (400 MHz, CHLOROFORM-d) δ |
| | 7.82 (s, 1H), 7.36 (s, 1H), 7.28 − 7.34 (m, |
| | 2H), 7.20 − 7.26 (m, 3H), 6.63 (s, 1H), 3.82 |
| | (q, J = 6.65 Hz, 2H), 2.80 (t, J = 7.43 Hz, |
| | 2H), 2.57 (s, 3H), 2.08 (quin, J = 7.14 Hz, |
| | 2H); LCMS: ESI [M + H]⁺ = 351.1 |
| 58 | ¹H NMR (400 MHz, CHLOROFORM-d) δ |
| | 7.28 − 7.34 (m, 2H), 7.19 − 7.25 (m, 3H), |
| | 6.58 (s, 1H), 4.89 (br. s., 1H), 3.59 (q, J = |
| | 6.39 Hz, 2H), 2.73 (t, J = 7.43 Hz, 2H), |
| | 2.53 (s, 3H), 2.00 (quin, J = 7.24 Hz, 2H), |
| | 1.42 (q, J = 3.39 Hz, 2H), 1.11 (q, J = 3.52 |
| | Hz, 2H); LCMS: ESI [M + H]⁺ = 339.1 |
| 59 | ¹H NMR (400 MHz, CHLOROFORM-d) δ |
| | 7.00 − 7.11 (m, 2H), 6.93 (br. s., 1H), 6.71 |
| | (s, 1H), 3.67 (q, J = 6.65 Hz, 2H), 2.86 (q, J = |
| | 7.43 Hz, 2H), 2.72 (t, J = 7.43 Hz, 2H), |
| | 2.54 (s, 3H), 2.01 (quin, J = 7.24 Hz, 2H), |
| | 1.36 (t, J = 7.63 Hz, 3H); LCMS: ESI |
| | [M + H]⁺ = 348.1 |
| 60 | ¹H NMR (400 MHz, CHLOROFORM-d) δ |
| | 6.96 − 7.11 (m, 2H), 6.88 − 6.95 (m, J = |
| | 8.22 Hz, 1H), 6.76 (s, 1H), 5.30 (br. s., 1H), |
| | 3.69 (q, J = 6.65 Hz, 2H), 2.71 (t, J = 7.43 |
| | Hz, 2H), 2.60 (s, 3H), 2.02 (quin, J = 7.34 |
| | Hz, 2H); LCMS: ESI [M + H]⁺ = 388.0 |
| 61 | ¹H NMR (400 MHz, CHLOROFORM-d) δ |
| | 7.36 − 7.41 (m, 2H), 7.33 (t, J = 7.63 Hz, |
| | 2H), 7.22 − 7.27 (m, 1H), 6.77 (s, 1H), 6.65 |
| | (d, J = 16.04 Hz, 1H), 6.32 − 6.41 (m, 1H), |
| | 4.44 (t, J = 5.87 Hz, 2H), 2.88 (q, J = 7.43 |
| | Hz, 2H), 2.55 (s, 3H), 1.38 (t, J = 7.63 Hz, |
| | 3H); LCMS: ESI [M + H]⁺ = 310.1 |
| 62 | ¹H NMR (400 MHz, CHLOROFORM-d) δ |
| | 7.19 (t, J = 7.83 Hz, 2H), 6.64 − 6.76 (m, |
| | 4H), 5.56 (br. s., 1H), 3.85 − 3.95 (m, 2H), |
| | 3.46 (t, J = 5.67 Hz, 2H), 2.86 − 2.94 (m, |
| | 2H), 2.52 (s, 3H), 1.40 (t, J = 7.63 Hz, 3H); |
| | LCMS: ESI [M + H]⁺ = 313.1 |
| 63 | ¹H NMR (400 MHz, CHLOROFORM-d) δ |
| | 7.95 (br. s., 1H), 7.42 (d, J = 4.30 Hz, 1H), |
| | 7.30 − 7.37 (m, 2H), 7.21 − 7.28 (m, 3H), |
| | 7.11 − 7.16 (m, 1H), 6.63 (s, 1H), 5.04 (br. |
| | s., 1H), 3.75 (q, J = 6.39 Hz, 2H), 2.81 (t, |
| | J = 7.43 Hz, 2H), 2.56 (s, 3H), 2.10 (quin, |
| | J = 7.04 Hz, 2H); LCMS: ESI [M + H]⁺ = |
| | 366.1 |
| 64 | ¹H NMR (400 MHz, CHLOROFORM-d) δ |
| | 7.28 − 7.34 (m, 2H), 6.98 (t, J = 7.43 Hz, |
| | 1H), 6.90 − 6.95 (m, 2H), 6.74 (s, 1H), 4.14 |
| | (t, J = 5.67 Hz, 2H), 3.85 (q, J = 6.26 Hz, |
| | 2H), 2.84 (q, J = 7.43 Hz, 2H), 2.54 (s, |
| | 3H), 2.20 (quin, J = 6.16 Hz, 2H), 1.36 (t, |
| | J = 7.63 Hz, 3H); LCMS: ESI [M + H]⁺ = |
| | 328.1 |
| 65 | ¹H NMR (400 MHz, CHLOROFORM-d) δ |
| | 7.39 (d, J = 9.00 Hz, 2H), 6.84 (d, J = 9.00 |
| | Hz, 2H), 6.79 (s, 1H), 4.16 − 4.22 (m, 2H), |
| | 4.05 (q, J = 5.48 Hz, 2H), 2.87 (q, J = 7.69 |
| | Hz, 2H), 2.54 (s, 3H), 1.37 (t, J = 7.63 Hz, |
| | 3H); LCMS: ESI [M + H]⁺ = 392.0, 394.0 |
| 66 | ¹H NMR (400 MHz, CHLOROFORM-d) δ |
| | 7.44 (d, J = 7.83 Hz, 2H), 7.33 − 7.39 (m, |
| | 2H), 7.21 − 7.26 (m, J = 7.04 Hz, 1H), 6.28 |
| | (s, 1H), 3.50 (q, J = 6.13 Hz, 2H), 2.82 (q, |
| | J = 7.43 Hz, 2H), 2.48 (s, 3H), 2.05 (t, J = |
| | 7.24 Hz, 2H), 1.42 (s, 6H), 1.34 (t, J = 7.63 |
| | Hz, 3H); LCMS: ESI [M + H]⁺ = 340.2 |
| 67 | ¹H NMR (400 MHz, CHLOROFORM-d) δ |
| | 7.06 − 7.11 (m, J = 8.22 Hz, 2H), 6.81 − |
| | 6.85 (m, J = 8.61 Hz, 2H), 6.62 (s, 1H), |
| | 5.07 (br. s., 1H), 3.80 − 3.88 (m, 2H), 2.83 − |
| | 2.93 (m, 4H), 2.51 (s, 3H), 1.37 (t, J = 7.63 |
| | Hz, 3H); LCMS: ESI [M + H]⁺ = 314.1 |
| 68 | ¹H NMR (400 MHz, CHLOROFORM-d) δ |
| | 7.92 (d, J = 7.43 Hz, 2H), 7.60 − 7.68 (m, |
| | 1H), 7.50 − 7.59 (m, J = 7.83 Hz, 2H), 6.73 |
| | (s, 1H), 4.04 − 4.11 (m, 2H), 3.49 − 3.56 (m, |
| | 2H), 2.80 (q, J = 7.69 Hz, 2H), 2.55 (s, |
| | 3H), 1.29 (t, J = 7.43 Hz, 3H); LCMS: ESI |
| | [M + H]⁺ = 362.1 |
| 69 | ¹H NMR (400 MHz, CHLOROFORM-d) δ |
| | 7.25 (d, J = 9.00 Hz, 2H), 6.84 (d, J = 9.00 |
| | Hz, 2H), 6.77 (br. s., 1H), 4.06 − 4.12 (m, |
| | 2H), 3.80 − 3.89 (m, 2H), 2.85 (q, J = 7.56 |
| | Hz, 2H), 2.54 (s, 3H), 2.15 − 2.24 (m, 2H), |
| | 1.35 (t, J = 7.63 Hz, 3H); LCMS: ESI |
| | [M + H]⁺ = 362.1 |
| 70 | ¹H NMR (400 MHz, CHLOROFORM-d) δ |
| | 7.06 − 7.10 (m, J = 8.22 Hz, 2H), 6.77 − |
| | 6.82 (m, J = 8.22 Hz, 2H), 6.59 (s, 1H), |
| | 3.66 (q, J = 6.26 Hz, 2H), 2.84 (q, J = 7.70 |
| | Hz, 2H), 2.69 (t, J = 7.43 Hz, 2H), 2.52 (s, |
| | 3H), 1.99 (quin, J = 7.04 Hz, 2H), 1.35 (t, |
| | J = 7.43 Hz, 3H); LCMS: ESI [M + H]⁺ = |
| | 328.1 |
| 71 | ¹H NMR (400 MHz, CHLOROFORM-d) δ |
| | 7.25 (d, J = 9.00 Hz, 2H), 6.83 (d, J = 9.00 |
| | Hz, 2H), 6.73 (s, 1H), 4.09 (t, J = 5.87 Hz, |
| | 2H), 3.82 (q, J = 6.26 Hz, 2H), 2.59 (s, |
| | 3H), 2.51 (s, 3H), 2.13 − 2.23 (m, 2H); |
| | LCMS: ESI [M + H]⁺ = 380.0 |
| 72 | ¹H NMR (400 MHz, CHLOROFORM-d) δ |
| | 7.25 (d, J = 9.00 Hz, 2H), 6.84 (d, J = 9.00 |
| | Hz, 2H), 6.81 (s, 1H), 5.72 (br. s., 1H), 4.11 |
| | (t, J = 5.87 Hz, 2H), 3.88 (q, J = 6.26 Hz, |
| | 2H), 2.61 (s, 3H), 2.21 (quin, J = 6.06 Hz, |
| | 2H); LCMS: ESI [M + H]⁺ = 402.1 |
| 73 | ¹H NMR (400 MHz, CHLOROFORM-d) δ |
| | 7.28 − 7.34 (m, 2H), 7.19 − 7.25 (m, 3H), |
| | 6.61 (s, 1H), 4.99 (t, J = 5.09 Hz, 1H), 3.65 |
| | (q, J = 6.39 Hz, 2H), 2.75 (t, J = 7.24 Hz, |
| | 2H), 2.54 (s, 3H), 2.03 (quin, J = 7.14 Hz, |
| | 2H), 1.56 (s, 6H); LCMS: ESI [M + H]⁺ = |
| | 341.2 |
| 74 | ¹H NMR (400 MHz, CHLOROFORM-d) δ |
| | 7.28 − 7.34 (m, 2H), 7.19 − 7.25 (m, 3H), |
| | 6.61 (s, 1H), 5.19 (br. s., 1H), 4.44 (s, 2H), |
| | 3.58 (q, J = 6.65 Hz, 2H), 3.12 (s, 3H), 2.74 |
| | (t, J = 7.24 Hz, 2H), 2.54 (s, 3H), 2.01 |
| | (quin, J = 7.14 Hz, 2H); LCMS: ESI |
| | [M + H]⁺ = 376.1 |
| 75 | ¹H NMR (400 MHz, CHLOROFORM-d) δ |
| | 7.28 − 7.34 (m, 2H), 7.23 (d, J = 7.04 Hz, |
| | 3H), 6.65 (s, 1H), 3.78 (s, 2H), 3.67 (q, J = |
| | 6.39 Hz, 2H), 2.77 (t, J = 7.43 Hz, 2H), |
| | 2.53 (s, 3H), 2.23 (s, 3H), 2.05 (quin, J = |
| | 7.14 Hz, 2H); LCMS: ESI [M + H]⁺ = 344.2 |
| 76 | ¹H NMR (400 MHz, CHLOROFORM-d) δ |
| | 7.28 − 7.34 (m, 2H), 7.19 − 7.25 (m, 3H), |
| | 6.65 (br. s., 1H), 3.65 (q, J = 6.65 Hz, 2H), |
| | 3.15 (q, J = 7.43 Hz, 2H), 2.75 (t, J = 7.24 |
| | Hz, 2H), 2.48 (s, 3H), 2.04 (quin, J = 7.24 |
| | Hz, 2H), 1.39 (t, J = 7.24 Hz, 3H); LCMS: |
| | ESI [M + H]⁺ = 344.2 |
| 77 | ¹H NMR (400 MHz, CHLOROFORM-d) δ |
| | 7.28 − 7.33 (m, 2H), 7.18 − 7.24 (m, J = |
| | 5.09 Hz, 3H), 6.56 (br. s., 1H), 3.61 (q, J = |
| | 6.39 Hz, 2H), 3.21 (br. s., 6H), 2.74 (t, J = |
| | 7.04 Hz, 2H), 2.40 (s, 3H), 2.03 (quin, J = |
| | 7.00 Hz, 2H); LCMS: ESI [M + H]⁺ = 327.2 |
| 78 | ¹H NMR (400 MHz, CHLOROFORM-d) δ |
| | 9.41 (s, 1H), 8.88 (d, J = 5.48 Hz, 1H), 8.29 |
| | (d, J = 5.09 Hz, 1H), 7.28 − 7.35 (m, 2H), |
| | 7.20 − 7.26 (m, 3H), 6.79 (br. s., 1H), 3.78 |
| | (q, J = 6.65 Hz, 2H), 2.81 (t, J = 7.24 Hz, |
| | 2H), 2.58 (s, 3H), 2.12 (quin, J = 7.14 Hz, |
| | 2H); LCMS: ESI [M + H]⁺ = 362.2 |
| 79 | ¹H NMR (400 MHz, CHLOROFORM-d) δ |
| | 9.42 (s, 1H), 8.90 (d, J = 5.09 Hz, 1H), 8.32 |
| | (d, J = 4.70 Hz, 1H), 7.25 (d, J = 8.61 Hz, |
| | 2H), 7.15 (d, J = 8.22 Hz, 2H), 6.88 (br. s., |
| | 1H), 3.79 (q, J = 6.65 Hz, 2H), 2.82 (t, J = |
| | 7.24 Hz, 2H), 2.59 (s, 3H), 2.12 (quin, J = |
| | 7.14 Hz, 2H); LCMS: ESI [M + H]⁺ = 446.1 |
| 80 | ¹H NMR (400 MHz, CHLOROFORM-d) δ |
| | 7.20 (t, J = 7.83 Hz, 2H), 6.81 (br. s., 1H), |
| | 6.73 (t, J = 7.24 Hz, 1H), 6.69 (d, J = 7.83 |
| | Hz, 2H), 3.80 (q, J = 6.00 Hz, 2H), 3.30 (t, |
| | J = 6.06 Hz, 2H), 2.90 (q, J = 7.70 Hz, 2H), |
| | 2.50 (s, 3H), 2.01 (quin, J = 6.26 Hz, 2H), |
| | 1.39 (t, J = 7.63 Hz, 3H); LCMS: ESI |
| | [M + H]⁺ = 327.2 |
| 81 | ¹H NMR (400 MHz, CHLOROFORM-d) δ |
| | 7.28 − 7.35 (m, 2H), 7.19 − 7.26 (m, 3H), |
| | 6.61 (br. s., 1H), 4.57 (s, 2H), 3.63 − 3.73 |
| | (m, J = 3.91 Hz, 2H), 3.55 (s, 3H), 2.72 − |
| | 2.81 (m, 2H), 2.54 (s, 3H), 1.99 − 2.09 (m, |
| | 2H); LCMS: ESI [M + H]⁺ = 328.2 |
| 82 | ¹H NMR (400 MHz, CHLOROFORM-d) δ |
| | 8.43 (t, J = 6.26 Hz, 2H), 7.21 − 7.26 (m, |
| | J = 7.04 Hz, 2H), 7.08 − 7.20 (m, 4H), 6.68 |
| | (br. s., 1H), 4.99 (br. s., 1H), 3.77 (q, J = |
| | 5.87 Hz, 2H), 2.80 (t, J = 7.04 Hz, 2H), |
| | 2.57 (s, 3H), 2.09 (quin, J = 6.65 Hz, 2H); |
| | LCMS: ESI [M + H]⁺ = 462.1 |
| 83 | ¹H NMR (400 MHz, CHLOROFORM-d) δ |
| | 8.38 − 8.44 (m, 2H), 7.08 − 7.15 (m, 4H), |
| | 6.79 (d, J = 8.22 Hz, 2H), 6.64 (s, 1H), 4.92 |
| | (br. s., 2H), 3.71 − 3.79 (m, 2H), 2.73 (t, J = |
| | 7.24 Hz, 2H), 2.57 (s, 3H), 2.00 − 2.08 (m, |
| | J = 6.85, 13.50 Hz, 2H); LCMS: ESI |
| | [M + H]⁺ = 394.2 |
| 84 | ¹H NMR (400 MHz, CHLOROFORM-d) δ |
| | 7.23 − 7.27 (m, 2H), 7.14 − 7.19 (m, 1H), |
| | 7.07 (d, J = 7.04 Hz, 2H), 6.90 (br. s., 1H), |
| | 3.70 (t, J = 6.26 Hz, 2H), 2.89 (q, J = 7.70 |
| | Hz, 2H), 2.52 (s, 3H), 1.94 − 2.01 (m, 1H), |
| | 1.48 − 1.56 (m, 1H), 1.38 (t, J = 7.63 Hz, |
| | 3H), 1.00 − 1.09 (m, 2H); LCMS: ESI |
| | [M + H]⁺ = 324.2 |
| 85 | ¹H NMR (400 MHz, CHLOROFORM-d) δ |
| | 8.36 (t, J = 6.26 Hz, 2H), 7.52 (s, 1H), 7.11 − |
| | 7.19 (m, 1H), 7.00 − 7.10 (m, 3H), 6.92 − |
| | 6.99 (m, J = 6.26 Hz, 1H), 5.74 (br. s., 1H), |
| | 3.15 (br. s., 1H), 2.14 − 2.21 (m, 1H), 1.47 |
| | (t, J = 6.65 Hz, 2H); LCMS: ESI [M + H]⁺ = |
| | 466.1 |
| 86 | ¹H NMR (400 MHz, CHLOROFORM-d) δ |
| | 7.28 − 7.34 (m, 2H), 7.20 − 7.26 (m, 3H), |
| | 6.58 (s, 1H), 4.88 (br. s., 1H), 3.65 − 3.72 |
| | (m, 2H), 2.80 − 2.91 (m, 4H), 2.77 (t, J = |
| | 7.24 Hz, 2H), 2.04 (quin, J = 6.85 Hz, 2H), |
| | 1.35 (dt, J = 1.57, 7.63 Hz, 6H); LCMS: |
| | ESI [M + H]⁺ = 326.2 |
| 87 | ¹H NMR (400 MHz, CHLOROFORM-d) δ |
| | 7.00 − 7.15 (m, 2H), 6.92 − 6.99 (m, J = |
| | 7.83 Hz, 1H), 6.66 (s, 1H), 5.38 (br. s., 1H), |
| | 2.96 − 3.14 (m, 3H), 2.47 (s, 3H), 2.07 (t, J = |
| | 7.83 Hz, 1H), 1.56 − 1.66 (m, 2H), 1.28 − |
| | 1.43 (m, 4H), 0.90 (t, J = 7.04 Hz, 3H); |
| | LCMS: ESI [M + H]⁺ = 406.1 |
| 88 | ¹H NMR (400 MHz, CHLOROFORM-d) δ |
| | 7.60 (s, 1H), 7.28 − 7.33 (m, J = 9.39 Hz, |
| | 1H), 7.08 − 7.17 (m, 1H), 7.05 − 7.08 (m, J = |
| | 2.74 Hz, 1H), 6.98 − 7.04 (m, J = 7.04 |
| | Hz, 1H), 6.76 (s, 1H), 6.50 − 6.53 (m, J = |
| | 1.57 Hz, 1H), 5.45 (s, 1H), 3.08 (br. s., 1H), |
| | 2.52 (s, 3H), 2.14 (t, J = 8.02 Hz, 1H), 1.33 − |
| | 1.42 (m, 2H); LCMS: ESI [M + H]⁺ = |
| | 384.1 |
| 89 | ¹H NMR (400 MHz, CHLOROFORM-d) δ |
| | 8.12 (d, J = 7.04 Hz, 1H), 7.59 (t, J = 7.83 |
| | Hz, 1H), 7.21 (d, J = 7.43 Hz, 1H), 7.04 − |
| | 7.15 (m, 2H), 6.94 − 7.00 (m, J = 7.43 Hz, |
| | 1H), 6.82 (s, 1H), 5.62 (br. s., 1H), 3.13 (br. |
| | s., 1H), 2.72 (s, 3H), 2.52 (s, 3H), 2.12 − |
| | 2.19 (m, J = 7.83 Hz, 1H), 1.41 (t, J = 7.00 |
| | Hz, 2H); LCMS: ESI [M + H]⁺ = 409.1 |
| 90 | ¹H NMR (400 MHz, CHLOROFORM-d) δ |
| | 8.34 − 8.40 (m, J = 7.83 Hz, 2H), 7.39 − |
| | 7.44 (m, J = 7.83 Hz, 2H), 7.30 − 7.36 (m, |
| | 2H), 7.22 − 7.26 (m, 3H), 6.63 (s, 1H), 4.98 |
| | (br. s., 1H), 3.76 (q, J = 6.39 Hz, 2H), 2.80 |
| | (t, J = 7.24 Hz, 2H), 2.57 (s, 3H), 2.09 |
| | (quin, J = 7.04 Hz, 2H); LCMS: ESI |
| | [M + H]⁺ = 394.1 |
| 91 | ¹H NMR (400 MHz, CHLOROFORM-d) δ |
| | 8.46 (s, 1H), 8.31 − 8.35 (m, 1H), 7.37 − |
| | 7.43 (m, 2H), 7.30 − 7.37 (m, 2H), 7.21 − |
| | 7.26 (m, 3H), 6.63 (s, 1H), 4.97 (br. s., 1H), |
| | 3.78 (q, J = 6.13 Hz, 2H), 2.81 (t, J = 7.43 |
| | Hz, 2H), 2.57 (s, 3H), 2.05 − 2.16 (m, 2H); |
| | LCMS: ESI [M + H]⁺ = 394.1 |
| 92 | ¹H NMR (400 MHz, CHLOROFORM-d) δ |
| | 8.34 (d, J = 6.65 Hz, 2H), 7.29 − 7.36 (m, |
| | 2H), 7.20 − 7.27 (m, 3H), 6.77 (d, J = 7.04 |
| | Hz, 2H), 6.61 (s, 1H), 4.89 (br. s., 1H), 3.74 − |
| | 3.81 (m, 2H), 3.04 (s, 6H), 2.80 (t, J = |
| | 7.04 Hz, 2H), 2.55 (s, 3H), 2.09 (quin, J = |
| | 6.95 Hz, 2H); LCMS: ESI [M + H]⁺ = 403.2 |
| 93 | ¹H NMR (400 MHz, CHLOROFORM-d) δ |
| | 7.27 − 7.33 (m, 2H), 7.21 (d, J = 7.04 Hz, |
| | 3H), 6.46 (s, 1H), 4.75 (br. s., 1H), 3.62 (q, |
| | J = 6.13 Hz, 2H), 3.14 (s, 3H), 2.68 − 2.78 |
| | (m, 3H), 2.45 (s, 3H), 2.02 (quin, J = 7.24 |
| | Hz, 2H), 0.76 − 0.83 (m, J = 6.26 Hz, 2H), |
| | 0.62 − 0.69 (m, 2H); LCMS: ESI [M + H]⁺ = |
| | 353.2 |
| 94 | ¹H NMR (400 MHz, CHLOROFORM-d) δ |
| | 7.28 − 7.34 (m, 2H), 7.22 (d, J = 6.65 Hz, |
| | 3H), 6.59 (s, 1H), 4.92 (br. s., 1H), 3.62 − |
| | 3.69 (m, 2H), 3.22 (dquin, J = 2.15, 8.17 |
| | Hz, 1H), 2.72 − 2.78 (m, 2H), 2.50 − 2.55 |
| | (m, 3H), 1.89 − 2.10 (m, 6H), 1.77 − 1.89 |
| | (m, J = 7.43, 7.43 Hz, 2H), 1.61 − 1.73 (m, |
| | 2H); LCMS: ESI [M + H]⁺ = 352.2 |
| 95 | ¹H NMR (400 MHz, CHLOROFORM-d) δ |
| | 7.29 − 7.35 (m, 2H), 7.24 (d, J = 6.65 Hz, |
| | 3H), 6.60 (br. s., 1H), 4.88 (br. s., 1H), 3.63 − |
| | 3.72 (m, J = 5.48 Hz, 2H), 2.73 − 2.81 (m, |
| | 2H), 2.53 (s, 3H), 1.95 − 2.09 (m, 4H), 1.84 |
| | (d, J = 12.52 Hz, 2H), 1.74 (d, J = 13.30 |
| | Hz, 1H), 1.54 − 1.69 (m, 3H), 1.24 − 1.49 |
| | (m, 3H); LCMS: ESI [M + H]⁺ = 366.2 |
| 96 | ¹H NMR (400 MHz, CHLOROFORM-d) δ |
| | 7.62 (br. s., 1H), 7.30 (d, J = 6.65 Hz, 2H), |
| | 7.18 − 7.25 (m, 3H), 6.70 (s, 1H), 5.80 (br. |
| | s., 1H), 5.27 (br. s., 1H), 3.64 − 3.73 (m, J = |
| | 5.87 Hz, 2H), 2.76 (t, J = 6.26 Hz, 2H), |
| | 2.57 (s, 3H), 1.99 − 2.10 (m, J = 6.46, 6.46 |
| | Hz, 2H); LCMS: ESI [M + H]⁺ = 327.1 |
| 97 | ¹H NMR (400 MHz, CHLOROFORM-d) δ |
| | 7.28 − 7.33 (m, 2H), 7.22 (d, J = 6.65 Hz, |
| | 3H), 6.63 (br. s., 1H), 3.67 (q, J = 6.52 Hz, |
| | 2H), 2.76 (t, J = 7.43 Hz, 2H), 2.68 − 2.73 |
| | (m, 2H), 2.52 (s, 3H), 2.05 (quin, J = 6.65 |
| | Hz, 2H), 1.22 − 1.33 (m, J = 5.09 Hz, 1H), |
| | 0.50 (d, J = 7.04 Hz, 2H), 0.30 (br. s., 2H); |
| | LCMS: ESI [M + H]⁺ = 338.2 |
| 98 | ¹H NMR (400 MHz, CHLOROFORM-d) δ |
| | 7.28 − 7.34 (m, 2H), 7.23 (d, J = 7.04 Hz, |
| | 3H), 6.64 (s, 1H), 5.12 (br. s., 1H), 4.02 (d, |
| | J = 1.96 Hz, 3H), 3.72 − 3.80 (m, 2H), 2.78 |
| | (t, J = 7.24 Hz, 2H), 2.57 (s, 3H), 2.06 |
| | (quin, J = 6.75 Hz, 2H); LCMS: ESI |
| | [M + H]⁺ = 342.1 |
| 99 | ¹H NMR (400 MHz, CHLOROFORM-d) δ |
| | 7.30 − 7.36 (m, 2H), 7.22 − 7.27 (m, J = |
| | 5.09 Hz, 3H), 7.18 (s, 1H), 5.04 (br. s., 1H), |
| | 3.68 − 3.76 (m, 2H), 2.84 − 2.90 (m, 2H), |
| | 2.75 − 2.84 (m, 2H), 2.07 (quin, J = 6.95 |
| | Hz, 2H), 1.36 (dt, J = 2.35, 7.63 Hz, 3H); |
| | LCMS: ESI [M + H]⁺ = 366.1 |
| 100 | ¹H NMR (400 MHz, CHLOROFORM-d) δ |
| | 7.28 − 7.34 (m, 2H), 7.19 − 7.25 (m, 3H), |
| | 6.52 (s, 1H), 4.92 (br. s., 1H), 3.94 (td, J = |
| | 6.85, 13.69 Hz, 1H), 3.63 (q, J = 6.26 Hz, |
| | 2H), 2.75 (t, J = 7.43 Hz, 2H), 2.50 (s, 3H), |
| | 2.02 (quin, J = 7.14 Hz, 2H), 1.43 (d, J = |
| | 6.65 Hz, 6H); LCMS: ESI [M + H]⁺ = 358.1 |
| 101 | ¹H NMR (400 MHz, CHLOROFORM-d) δ |
| | 8.10 (br. s., 1H), 7.83 (d, J = 4.70 Hz, 1H), |
| | 7.28 − 7.31 (m, 2H), 7.19 − 7.24 (m, 4H), |
| | 6.58 (s, 1H), 4.91 (br. s., 1H), 3.71 (q, J = |
| | 6.26 Hz, 2H), 2.75 (t, J = 7.24 Hz, 2H), |
| | 2.52 (s, 3H), 2.05 (quin, J = 6.95 Hz, 2H); |
| | LCMS: ESI [M + H]⁺ = 366.1 |
| 102 | ¹H NMR (400 MHz, CHLOROFORM-d) δ |
| | 8.14 (s, 1H), 7.44 − 7.49 (m, 1H), 7.29 − |
| | 7.36 (m, 2H), 7.21 − 7.26 (m, J = 7.04 Hz, |
| | 3H), 7.04 (s, 1H), 6.61 (s, 1H), 4.95 (br. s., |
| | 1H), 3.71 (q, J = 5.87 Hz, 2H), 2.78 (t, J = |
| | 7.24 Hz, 2H), 2.55 (s, 3H), 2.07 (quin, J = |
| | 6.85 Hz, 2H); LCMS: ESI [M + H]⁺ = 350.1 |
| 103 | ¹H NMR (400 MHz, CHLOROFORM-d) δ |
| | 7.10 − 7.16 (m, J = 7.43 Hz, 2H), 6.82 − |
| | 6.88 (m, J = 7.83 Hz, 2H), 6.47 (s, 1H), |
| | 4.84 (br. s., 1H), 3.80 (s, 3H), 3.64 (q, J = |
| | 6.00 Hz, 2H), 2.70 (t, J = 6.85 Hz, 2H), |
| | 2.55 (s, 3H), 2.50 (s, 3H), 1.93 − 2.04 (m, |
| | 2H); LCMS: ESI [M + H]⁺ = 360.1 |
| 104 | ¹H NMR (400 MHz, CHLOROFORM-d) δ |
| | 9.25 (s, 1H), 8.46 (d, J = 8.22 Hz, 1H), 7.53 − |
| | 7.60 (m, 1H), 7.28 − 7.34 (m, J = 6.65 Hz, |
| | 2H), 7.19 − 7.25 (m, J = 6.26 Hz, 3H), 6.71 |
| | (s, 1H), 5.23 (br. s., 1H), 3.79 (q, J = 6.13 |
| | Hz, 2H), 2.79 (t, J = 6.65 Hz, 2H), 2.58 (s, |
| | 3H), 2.08 (quin, J = 7.04 Hz, 2H); LCMS: |
| | ESI [M + H]⁺ = 362.1 |
| 105 | ¹H NMR (400 MHz, CHLOROFORM-d) δ |
| | 9.68 (s, 1H), 8.75 (s, 1H), 8.63 (s, 1H), 7.28 − |
| | 7.35 (m, J = 6.26 Hz, 2H), 7.19 − 7.26 (m, |
| | 3H), 6.68 (s, 1H), 5.17 (br. s, 1H), 3.78 (q, |
| | J = 6.00 Hz, 2H), 2.80 (t, J = 6.65 Hz, 2H), |
| | 2.57 (s, 3H), 2.10 (quin, J = 7.14 Hz, 2H); |
| | LCMS: ESI [M + H]⁺ = 362.1 |
| 106 | ¹H NMR (400 MHz, CHLOROFORM-d) δ |
| | 7.28 − 7.36 (m, J = 6.65 Hz, 2H), 7.17 − |
| | 7.26 (m, J = 7.04 Hz, 3H), 6.59 (s, 1H), |
| | 5.01 (br. s., 1H), 3.62 − 3.75 (m, 3H), 2.77 |
| | (t, J = 7.24 Hz, 2H), 2.53 (s, 3H), 2.41 − |
| | 2.51 (m, 2H), 2.28 − 2.38 (m, J = 8.22 Hz, |
| | 2H), 2.03 − 2.11 (m, J = 7.04 Hz, 3H), 1.87 − |
| | 1.98 (m, J = 9.00 Hz, 1H); LCMS: ESI |
| | [M + H]⁺ = 338.1 |
| 107 | ¹H NMR (400 MHz, CHLOROFORM-d) δ |
| | 7.13 − 7.24 (m, 3H), 7.10 (br. s., 1H), 3.16 |
| | (br. s., 1H), 2.59 (s, 3H), 2.10 (br. s., 1H), |
| | 1.39 − 1.48 (m, J = 5.87 Hz, 2H); LCMS: |
| | ESI [M + H]⁺ = 361.0 |
| 108 | ¹H NMR (400 MHz, CHLOROFORM-d) δ |
| | 7.28 − 7.35 (m, J = 6.26 Hz, 2H), 7.19 − |
| | 7.25 (m, 3H), 6.58 (s, 1H), 4.89 (br. s., 1H), |
| | 3.81 (s, 2H), 3.62 − 3.73 (m, J = 5.87 Hz, |
| | 2H), 2.68 − 2.80 (m, 6H), 2.53 (s, 3H), 1.97 − |
| | 2.08 (m, J = 6.65 Hz, 2H), 1.82 (br. s., |
| | 4H); LCMS: ESI [M + H]⁺ = 367.2 |
| 109 | ¹H NMR (400 MHz, CHLOROFORM-d) δ |
| | 7.30 (d, J = 6.65 Hz, 2H), 7.19 − 7.25 (m, |
| | 3H), 6.60 (s, 1H), 4.96 (br. s., 1H), 3.69 − |
| | 3.72 (m, J = 2.74 Hz, 4H), 2.76 (t, J = 7.04 |
| | Hz, 2H), 2.54 (s, 3H), 2.47 (br. s., 6H), 1.99 − |
| | 2.07 (m, 2H); LCMS: ESI [M + H]⁺ = |
| | 341.2 |
| 110 | ¹H NMR (400 MHz, CHLOROFORM-d) δ |
| | 8.48 (br. s., 1H), 7.28 − 7.35 (m, J = 6,65 |
| | Hz, 2H), 7.19 − 7.26 (m, J = 6.26 Hz, 3H), |
| | 6.97 (s, 1H), 6.65 (s, 1H), 5.06 (br. s., 1H), |
| | 3.72 − 3.80 (m, 2H), 2.74 − 2.82 (m, 2H), |
| | 2.57 (s, 3H), 2.03 − 2.12 (m, J = 6.65, 6.65 |
| | Hz, 2H); LCMS: ESI [M + H]⁺ = 351.1 |
| 111 | ¹H NMR (400 MHz, CHLOROFORM-d) δ |
| | 7.30 (d, J = 6.65 Hz, 2H), 7.19 − 7.25 (m, J = |
| | 5.48 Hz, 3H), 6.65 (br. s., 1H), 6.58 (br. |
| | s., 1H), 5.13 (br. s., 1H), 3.70 − 3.78 (m, J = |
| | 5.87 Hz, 2H), 2.76 (t, J = 6.85 Hz, 2H), |
| | 2.55 (s, 3H), 2.50 (s, 3H), 2.05 (quin, J = |
| | 6.95 Hz, 2H); LCMS: ESI [M + H]⁺ = 365.2 |
| 112 | ¹H NMR (400 MHz, CHLOROFORM-d) δ |
| | 10.78 (br. s., 1H), 7.59 (br. s., 1H), 7.20 (br. |
| | s., 5H), 6.90 (br. s., 1H), 6.56 (br. s., 1H), |
| | 4.93 (br. s., 1H), 3.60 − 3.69 (m, J = 6.26 |
| | Hz, 2H), 2.73 (t, J = 6.26 Hz, 2H), 2.50 (s, |
| | 3H), 1.96 − 2.07 (m, 2H); LCMS: ESI |
| | [M + H]⁺ = 350.2 |
| 113 | ¹H NMR (400 MHz, CHLOROFORM-d) δ |
| | 7.59 (s, 1H), 7.28 − 7.35 (m, 2H), 7.20 − |
| | 7.26 (m, 3H), 7.16 − 7.18 (m, 1H), 6.60 (s, |
| | 1H), 6.51 − 6.56 (m, J = 1.57, 3.33, 3.33 |
| | Hz, 1H), 4.98 (br. s., 1H), 3.69 − 3.76 (m, |
| | 2H), 2.78 (t, J = 7.43 Hz, 2H), 2.54 (s, 3H), |
| | 2.02 − 2.12 (m, 2H); LCMS: ESI [M + H]⁺ = |
| | 350.1 |
| 114 | ¹H NMR (400 MHz, CHLOROFORM-d) δ |
| | 8.01 (s, 1H), 7.27 (d, J = 2.35 Hz, 1H), 7.09 − |
| | 7.18 (m, 1H), 7.01 − 7.08 (m, 1H), 6.99 |
| | (br. s., 1H), 6.94 (s, 1H), 6.75 (s, 1H), 5.38 |
| | (br. s., 1H), 3.06 − 3.12 (m, 1H), 2.52 (s, |
| | 3H), 2.11 (t, J = 7.83 Hz, 1H), 1.34 − 1.42 |
| | (m, 2H); LCMS: ESI [M + H]⁺ = 384.1 |
| 115 | ¹H NMR (400 MHz, CHLOROFORM-d) δ |
| | 7.07 − 7.17 (m, 2H), 7.01 (br. s., 1H), 6.73 |
| | (s, 1H), 5.46 (br. s., 1H), 3.68 (quin, J = |
| | 8.41 Hz, 1H), 3.04 − 3.11 (m, J = 1.96 Hz, |
| | 1H), 2.48 (s, 3H), 2.37 − 2.47 (m, 2H), 2.24 − |
| | 2.36 (m, 2H), 2.12 (t, J = 7.63 Hz, 1H), |
| | 1.97 − 2.06 (m, 1H), 1.78 − 1.89 (m, 1H), |
| | 1.35 (dd, J = 4.89, 7.24 Hz, 2H); LCMS: |
| | ESI [M + H]⁺ = 372.1 |
| 116 | ¹H NMR (400 MHz, CHLOROFORM-d) δ |
| | 7.28 − 7.34 (m, 2H), 7.18 − 7.25 (m, J = |
| | 1.37, 6.46 Hz, 3H), 6.62 (s, 1H), 4.95 (br. |
| | s., 1H), 3.65 − 3.72 (m, 2H), 3.24 (d, J = |
| | 2.35 Hz, 3H), 2.75 (t, J = 7.63 Hz, 2H), |
| | 2.54 (s, 3H), 1.99 − 2.08 (m, 2H), 1.63 (d, J = |
| | 1.96 Hz, 6H); LCMS: ESI [M + H]⁺ = |
| | 356.2 |
| 117 | ¹H NMR (400 MHz, CHLOROFORM-d) δ |
| | 7.22 − 7.26 (m, 2H), 7.14 (d, J = 8.22 Hz, |
| | 2H), 6.72 (s, 1H), 5.15 (br. s., 1H), 4.03 (s, |
| | 3H), 3.73 − 3.80 (m, 2H), 2.77 (t, J = 7.43 |
| | Hz, 2H), 2.59 (s, 3H), 2.00 − 2.09 (m, 2H); |
| | LCMS: ESI [M + H]⁺ = 426.1 |
| 118 | ¹H NMR (400 MHz, CHLOROFORM-d) δ |
| | 8.14 (s, 1H), 7.46 (d, J = 1.57 Hz, 1H), 7.12 − |
| | 7.18 (m, J = 7.04 Hz, 2H), 7.04 (s, 1H), |
| | 6.83 − 6.90 (m, J = 7.04 Hz, 2H), 6.57 (s, |
| | 1H), 4.88 (br. s., 1H), 3.81 (d, J = 1.57 Hz, |
| | 3H), 3.66 − 3.75 (m, 2H), 2.73 (t, J = 7.24 |
| | Hz, 2H), 2.55 (s, 3H), 2.03 (quin, J = 6.95 |
| | Hz, 2H); LCMS: ESI [M + H]⁺ = 380.2 |
| 119 | ¹H NMR (400 MHz, CHLOROFORM-d) δ |
| | 9.25 (dd, J = 2.15, 6.06 Hz, 2H), 7.32 − |
| | 7.38 (m, 2H), 7.24 − 7.30 (m, 3H), 6.66 (s, |
| | 1H), 5.10 (br. s., 1H), 3.71 − 3.80 (m, 2H), |
| | 2.81 (t, J = 7.24 Hz, 2H), 2.58 (s, 3H), 2.04 − |
| | 2.16 (m, 2H); LCMS: ESI [M + H]⁺ = |
| | 367.2 |
| 120 | ¹H NMR (400 MHz, CHLOROFORM-d) δ |
| | 7.28 − 7.34 (m, 2H), 7.19 − 7.26 (m, 3H), |
| | 6.59 (s, 1H), 4.94 (br. s., 1H), 4.23 (dt, J = |
| | 1.57, 8.02 Hz, 1H), 4.01 − 4.08 (m, 2H), |
| | 3.92 − 3.99 (m, 1H), 3.56 − 3.69 (m, 3H), |
| | 2.75 (t, J = 7.43 Hz, 2H), 2.54 (s, 3H), 2.35 − |
| | 2.45 (m, 1H), 2.25 − 2.35 (m, 1H), 2.03 |
| | (quin, J = 7.14 Hz, 2H); LCMS: ESI |
| | [M + H]⁺ = 354.2 |
| 121 | ¹H NMR (400 MHz, CHLOROFORM-d) δ |
| | 8.47 − 8.49 (m, 1H), 7.19 − 7.24 (m, 2H), |
| | 7.10 − 7.16 (m, 2H), 6.95 (t, J = 1.57 Hz, |
| | 1H), 6.74 (s, 1H), 5.24 (br. s., 1H), 3.71 − |
| | 3.78 (m, 2H), 2.75 (t, J = 7.24 Hz, 2H), |
| | 2.56 (s, 3H), 1.99 − 2.08 (m, 2H); LCMS: |
| | ESI [M + H]⁺ = 435.1 |
| 122 | ¹H NMR (400 MHz, CHLOROFORM-d) o |
| | 7.19 − 7.26 (m, 2H), 7.10 − 7.17 (m, 2H), |
| | 6.72 (s, 1H), 6.59 (s, 1H), 5.17 (br. s., 1H), |
| | 3.74 (q, J = 5.87 Hz, 2H), 2.75 (t, J = 7.43 |
| | Hz, 2H), 2.56 (s, 3H), 2.48 − 2.52 (m, 3H), |
| | 1.99 − 2.08 (m, 2H); LCMS: ESI [M + H]⁺ = |
| | 449.2 |
| 123 | ¹H NMR (400 MHz, CHLOROFORM-d) δ |
| | 8.80 − 8.85 (m, 1H), 8.43 (d, J = 7.83 Hz, |
| | 1H), 7.78 − 7.84 (m, J = 7.63, 7.63 Hz, 1H), |
| | 7.35 (t, J = 6.06 Hz, 1H), 7.23 (d, J = 7.83 |
| | Hz, 2H), 7.13 (d, J = 7.80 Hz, 2H), 6.72 (s, |
| | 1H), 5.15 (br. s., 1H), 3.79 (q, J = 5.61 Hz, |
| | 2H), 2.78 (t, J = 7.43 Hz, 2H), 2.56 (s, 3H), |
| | 2.07 (quin, J = 7.14 Hz, 2H); LCMS: ESI |
| | [M + H]⁺ = 445.2 |
| 124 | ¹H NMR (400 MHz, CHLOROFORM-d) δ |
| | 7.23 (d, J = 6.65 Hz, 2H), 7.12 − 7.17 (m, |
| | 2H), 6.57 (s, 1H), 4.99 (br. s., 1H), 3.61 − |
| | 3.68 (m, 2H), 2.74 (t, J = 7.43 Hz, 2H), |
| | 2.54 (d, J = 2.35 Hz, 3H), 2.50 (s, 3H), 1.96 − |
| | 2.06 (m, 2H); LCMS: ESI [M + H]⁺ = |
| | 414.1 |
| 125 | ¹H NMR (400 MHz, CHLOROFORM-d) δ |
| | 9.25 (d, J = 3.52 Hz, 1H), 9.29 (d, J = 3.52 |
| | Hz, 1H), 7.25 (dd, J = 3.13, 8.61 Hz, 2H), |
| | 7.13 − 7.20 (m, 2H), 6.68 (s, 1H), 4.99 (br. |
| | s., 1H), 3.71 − 3.79 (m, 2H), 2.80 (dt, J = |
| | 3.52, 7.43 Hz, 2H), 2.58 (d, J = 2.74 Hz, |
| | 3H), 2.09 (ttd, J = 3.62, 7.21, 10.81 Hz, |
| | 2H); LCMS: ESI [M + H]⁺ = 451.1 |
| 126 | 7.16 (m, 2H), 6.64 (s, 1H), 4.97 (br. s., 1H), |
| | ¹H NMR (400 MHz, CHLOROFORM-d) δ |
| | 8.39 − 8.46 (m, 2H), 7.30 − 7.36 (m, 2H), |
| | 7.22 − 7.27 (m, J = 7.43 Hz, 3H), 7.09 − |
| | 3.77 (q, J = 6.13 Hz, 2H), 2.80 (t, J = 7.43 |
| | Hz, 2H), 2.57 (s, 3H), 2.10 (quin, J = 7.14 |
| | Hz, 2H); LCMS: ESI [M + H]⁺ = 378.1 |
| 127 | ¹H NMR (400 MHz, CHLOROFORM-d) δ |
| | 7.31 − 7.42 (m, 2H), 7.08 (d, J = 7.04 Hz, |
| | 1H), 6.66 (s, 1H), 5.10 (br. s., 1H), 3.86 (br. |
| | s., 2H), 2.97 (br. s., 2H), 2.82 − 2.92 (m, J = |
| | 7.04 Hz, 2H), 2.54 (s, 3H), 1.38 (t, J = 6.85 |
| | Hz, 3H); LCMS: ESI [M + H]⁺ = 366.1, |
| | 368.0 |
| 128 | ¹H NMR (400 MHz, CHLOROFORM-d) δ |
| | 8.91 (d, J = 1.96 Hz, 1H), 8.12 (t, J = 1.96 |
| | Hz, 1H), 7.29 − 7.35 (m, 2H), 7.21 − 7.27 |
| | (m, 3H), 6.65 (s, 1H), 5.06 (br. s., 1H), 3.76 |
| | (q, J = 5.61 Hz, 2H), 2.79 (t, J = 7.24 Hz, |
| | 2H), 2.55 (s, 3H), 2.08 (quin, J = 7.14 Hz, |
| | 2H); LCMS: ESI [M + H]⁺ = 367.1 |
| 129 | ¹H NMR (400 MHz, CHLOROFORM-d) δ |
| | 11.31 (br. s., 1H), 8.13 (s, 1H), 7.32 − 7.41 |
| | (m, J = 7.04 Hz, 2H), 7.27 − 7.32 (m, J = |
| | 3.13 Hz, 3H), 6.65 (s, 1H), 5.12 (br. s., 1H), |
| | 3.75 (br. s., 2H), 2.75 − 2.85 (m, J = 5.87 |
| | Hz, 2H), 2.58 (s, 3H), 2.03 − 2.15 (m, J = |
| | 5.87 Hz, 2H); LCMS: ESI [M + H]⁺ = 351.1 |
| 130 | ¹H NMR (400 MHz, CHLOROFORM-d) δ |
| | 8.22 − 8.27 (m, 2H), 7.29 − 7.35 (m, 2H), |
| | 7.24 (d, J = 7.04 Hz, 3H), 6.61 (s, 1H), 4.98 |
| | (br. s., 1H), 3.73 (q, J = 5.48 Hz, 2H), 2.79 |
| | (t, J = 7.24 Hz, 2H), 2.55 (s, 3H), 2.02 − |
| | 2.13 (m, 2H); LCMS: ESI [M + H]⁺ = 350.1 |
| 131 | ¹H NMR (400 MHz, CHLOROFORM-d) δ |
| | 8.53 (s, 1H), 7.84 (s, 1H), 7.30 − 7.36 (m, |
| | 2H), 7.21 − 7.27 (m, 3H), 7.13 (s, 1H), 6.62 |
| | (s, 1H), 5.31 (br. s., 1H), 3.65 − 3.73 (m, |
| | 2H), 2.78 (t, J = 7.24 Hz, 2H), 2.55 (s, 3H), |
| | 2.07 (quin, J = 6.95 Hz, 2H); LCMS: ESI |
| | [M + H]⁺ = 350.1 |
| 132 | ¹H NMR (400 MHz, CHLOROFORM-d) δ |
| | 7.29 − 7.37 (m, J = 7.43 Hz, 2H), 7.20 − |
| | 7.27 (m, J = 6.65 Hz, 3H), 7.09 (s, 1H), |
| | 6.60 (s, 1H), 6.14 (br. s., 1H), 4.95 (br. s., |
| | 1H), 3.67 − 3.78 (m, J = 5.09 Hz, 2H), 2.78 |
| | (t, J = 6.85 Hz, 2H), 2.54 (s, 3H), 2.45 (s, |
| | 3H), 2.00 − 2.13 (m, J = 6.26 Hz, 2H); |
| | LCMS: ESI [M + H]⁺ = 364.1 |
| 133 | ¹H NMR (400 MHz, CHLOROFORM-d) δ |
| | 8.12 (s, 1H), 7.98 (s, 1H), 7.29 − 7.38 (m, |
| | 2H), 7.24 (d, J = 6.97 Hz, 3H), 6.60 (d, J = |
| | 1.22 Hz, 1H), 4.92 (br. s., 1H), 3.95 (s, 3H), |
| | 3.72 (q, J = 6.81 Hz, 2H), 2.78 (t, J = 7.34 |
| | Hz, 2H), 2.54 (d, J = 1.10 Hz, 3H), 2.07 |
| | (quin, J = 7.21 Hz, 2H); LCMS: ESI |
| | [M + H]⁺ = 364.2 |
| 134 | ¹H NMR (400 MHz, CHLOROFORM-d) δ |
| | 7.28 − 7.34 (m, 2H), 7.19 − 7.25 (m, 3H), |
| | 6.60 (d, J = 1.10 Hz, 1H), 5.16 (br. s., 1H), |
| | 3.60 − 3.72 (m, 2H), 2.76 (t, J = 7.46 Hz, |
| | 2H), 2.68 (d, J = 7.21 Hz, 2H), 2.53 (d, J = |
| | 1.10 Hz, 3H), 2.29 (quind, J = 6.84, 13.62 |
| | Hz, 1H), 2.03 (quin, J = 7.21 Hz, 2H), 0.98 |
| | (d, J = 6.60 Hz, 6H LCMS: ESI [M + H]⁺ = |
| | 340.2 |
| 135 | ¹H NMR (400 MHz, CHLOROFORM-d) δ |
| | 7.28 − 7.36 (m, 2H), 7.18 − 7.26 (m, 3H), |
| | 6.60 (d, J = 1.22 Hz, 1H), 5.09 (q, J = 6.72 |
| | Hz, 1H), 5.00 (br. s., 1H), 3.61 − 3.76 (m, |
| | 2H), 2.77 (t, J = 7.46 Hz, 2H), 2.55 (d, J = |
| | 1.10 Hz, 3H), 2.05 (quin, J = 7.21 Hz, 2H), |
| | 1.91 (d, J = 6.72 Hz, 3H); LCMS: ESI |
| | [M + H]⁺ = 346.1 |
| 136 | ¹H NMR (400 MHz, CHLOROFORM-d) δ |
| | 7.13 − 7.01 (m, 3H), 6.58 (s, 1H), 4.84 (s, |
| | 1H), 3.66 (q, J = 6.7 Hz, 2H), 2.87 − 2.77 |
| | (m, 2H), 2.71 (t, J = 7.6 Hz, 2H), 2.52 (d, |
| | J = 2.8 Hz, 3H), 2.26 (d, J = 2.8 Hz, 3H), |
| | 2.00 (t, J = 7.5 Hz, 2H), 1.33 (td, J = 7.6, |
| | 2.8 Hz, 3H); LCMS: ESI [M + H]⁺ = 410.2 |
| 137 | ¹H NMR (400 MHz, CHLOROFORM-d) δ |
| | 7.16 − 7.39 (m, 7H), 6.94 (t, J = 8.61 Hz, |
| | 2H), 6.56 (s, 1H), 4.94 (br. s., 1H), 4.08 (s, |
| | 2H), 3.61 (q, J = 6.65 Hz, 2H), 2.71 (t, J = |
| | 7.43 Hz, 2H), 2.52 (s, 3H), 1.98 (quin, J = |
| | 7.24 Hz, 2H); LCMS: ESI [M + H]⁺ = 392.2 |
| 138 | ¹H NMR (400 MHz, CHLOROFORM-d) δ |
| | 7.26 − 7.34 (m, 6H), 7.14 − 7.26 (m, 4H), |
| | 6.59 (s, 1H), 5.13 (br. s., 1H), 3.66 (q, J = |
| | 6.52 Hz, 2H), 3.14 (s, 4H), 2.76 (t, J = 7.43 |
| | Hz, 2H), 2.53 (s, 3H), 2.03 (quin, J = 7.04 |
| | Hz, 2H); LCMS: ESI [M + H]⁺ = 388.2 |
| 139 | ¹H NMR (400 MHz, CHLOROFORM-d) δ |
| | 7.28 − 7.34 (m, 2H), 7.22 (d, J = 7.43 Hz, |
| | 3H), 6.60 (br. s., 2H), 4.91 (br. s., 1H), 3.69 |
| | (q, J = 6.91 Hz, 2H), 2.77 (t, J = 7.43 Hz, |
| | 2H), 2.70 (s, 3H), 2.53 (s, 3H), 2.30 (s, 3H), |
| | 2.01 − 2.12 (m, 2H); LCMS: ESI [M + H]⁺ = |
| | 378.2 |
| 140 | ¹H NMR (400 MHz, CHLOROFORM-d) δ |
| | 7.28 − 7.33 (m, 2H), 7.17 − 7.24 (m, 3H), |
| | 6.61 (s, 1H), 6.00 (s, 1H), 4.17 (s, 2H), 3.61 |
| | (q, J = 6.65 Hz, 2H), 2.71 (t, J = 7.43 Hz, |
| | 2H), 2.52 (s, 3H), 2.30 − 2.36 (m, 3H), 1.98 |
| | (quin, J = 7.24 Hz, 2H); LCMS: ESI |
| | [M + H]⁺ = 379.2 |
| 141 | ¹H NMR (400 MHz, CHLOROFORM-d) δ |
| | 8.11 (br. s., 1H), 7.29 − 7.36 (m, 2H), 7.21 − |
| | 7.26 (m, 3H), 6.63 (br. s., 1H), 3.70 (q, J = |
| | 6.65 Hz, 2H), 2.78 (t, J = 7.24 Hz, 2H), |
| | 2.50 − 2.59 (m, 6H), 1.99 − 2.13 (m, 2H); |
| | LCMS: ESI [M + H]⁺ = 365.2 |
| 142 | ¹H NMR (400 MHz, CHLOROFORM-d) δ |
| | 7.40 (d, J = 7.43 Hz, 2H), 7.14 − 7.31 (m, |
| | 8H), 6.59 (br. s., 1H), 4.40 (s, 2H), 3.60 (q, |
| | J = 6.39 Hz, 2H), 2.70 (t, J = 7.24 Hz, 2H), |
| | 2.46 (s, 3H), 1.98 (quin, J = 7.24 Hz, 2H); |
| | LCMS: ESI [M + H]⁺ = 406.2 |
| 143 | ¹H NMR (400 MHz, CHLOROFORM-d) δ |
| | 8.87 (s, 1H), 7.28 − 7.35 (m, 2H), 7.18 − |
| | 7.26 (m, 3H), 6.66 (s, 1H), 3.81 (q, J = 6.60 |
| | Hz, 2H), 2.80 (t, J = 7.40 Hz, 2H), 2.58 (s, |
| | 3H), 2.09 (quin, J = 7.12 Hz, 2H); LCMS: |
| | ESI [M + H]⁺ = 352.1 |
| 144 | ¹H NMR (400 MHz, CHLOROFORM-d) δ |
| | 9.06 (s, 1H), 8.42 (d, J = 8.22 Hz, 1H), 7.91 |
| | (d, J = 8.22 Hz, 1H), 7.20 − 7.31 (m, 1H), |
| | 7.12 (q, J = 8.87 Hz, 1H), 7.00 (br. s., 1H), |
| | 6.38 (br. s., 1H), 3.14 (br. s., 1H), 2.80 (s, |
| | 3H), 2.12 − 2.24 (m, 1H), 1.43 (t, J = 7.04 |
| | Hz, 2H); LCMS: ESI [M + H]⁺ = 464.1 |
| 145 | ¹H NMR (400 MHz, CHLOROFORM-d) δ |
| | 9.06 (s, 1H), 8.52 (d, J = 8.22 Hz, 1H), 8.04 |
| | (d, J = 7.83 Hz, 1H), 7.27 − 7.34 (m, 2H), |
| | 7.23 (d, J = 7.04 Hz, 3H), 6.13 (br. s., 1H), |
| | 3.75 − 3.85 (m, 2H), 2.78 − 2.83 (m, 5H), |
| | 2.12 (quin, J = 7.24 Hz, 2H); LCMS: ESI |
| | [M + H]⁺ = 430.0 |
| 146 | ¹H NMR (400 MHz, CHLOROFORM-d) |
| | 8.65 (d, J = 2.74 Hz, 1H), 8.33 (dd, J = |
| | 4.70, 8.61 Hz, 1H), 7.38 (dt, J = 2.93, 8.31 |
| | Hz, 1H), 7.24 (d, J = 9.00 Hz, 1H), 7.06 − |
| | 7.17 (m, 1H), 7.00 (br. s., 1H), 6.32 (br. s., |
| | 1H), 3.13 (br. s., 1H), 2.80 (s, 3H), 2.12 − |
| | 2.26 (m, 1H), 1.35 − 1.50 (m, 2H); LCMS: |
| | ESI [M + H]⁺ = 414.1 |
| 147 | ¹H NMR (400 MHz, CHLOROFORM-d) δ |
| | 8.65 (d, J = 2.74 Hz, 1H), 8.43 (dd, J = |
| | 4.70, 8.61 Hz, 1H), 7.50 (dt, J = 2.74, 8.41 |
| | Hz, 1H), 7.27 − 7.34 (m, 2H), 7.20 − 7.26 |
| | (m, 3H), 6.07 (br. s., 1H), 3.79 (q, J = 6.52 |
| | Hz, 2H), 2.75 − 2.85 (m, 5H), 2.11 (t, J = |
| | 7.43 Hz, 2H); LCMS: ESI [M + H]⁺ = 380.1 |
| 148 | ¹H NMR (400 MHz, CHLOROFORM-d) δ |
| | 9.44 (s, 1H), 8.80 (d, J = 5.09 Hz, 1H), 8.21 |
| | (d, J = 5.09 Hz, 1H), 7.23 − 7.31 (m, 1H), |
| | 7.14 (q, J = 8.74 Hz, 1H), 7.01 (d, J = 6.26 |
| | Hz, 1H), 6.41 (br. s., 1H), 3.16 (br. s., 1H), |
| | 2.83 (s, 3H), 2.12 − 2.29 (m, 1H), 1.45 (t, |
| | J = 7.04 Hz, 2H); LCMS: ESI [M + H]⁺ = |
| | 397.1 |
| 149 | ¹H NMR (400 MHz, CHLOROFORM-d) δ |
| | 9.45 (s, 1H), 8.92 (d, J = 5.09 Hz, 1H), 8.31 |
| | (d, J = 5.09 Hz, 1H), 7.31 − 7.37 (m, 2H), |
| | 7.23 − 7.29 (m, 3H), 6.17 (br. s., 1H), 3.82 |
| | (d, J = 6.65 Hz, 2H), 2.81 − 2.90 (m, 5H), |
| | 2.06 − 2.19 (m, 2H); LCMS: ESI [M + H]⁺ = |
| | 363.1 |
| 150 | ¹H NMR (400 MHz, METHANOL-d4) δ |
| | 6.99 − 7.12 (m, 2H), 6.92 (br. s., 1H), 6.86 |
| | (s, 1H), 4.11 (q, J = 7.04 Hz, 2H), 2.96 (td, |
| | J = 3.86, 7.53 Hz, 1H), 2.38 (s, 3H), 1.89 − |
| | 2.04 (m, 1H), 1.32 (td, J = 5.14, 9.68 Hz, |
| | 1H), 1.21 − 1.26 (m, 1H), 1.09 − 1.16 (m, |
| | 3H); LCMS: ESI [M + H]⁺ = 362.1 |
| 151 | ¹H NMR (400 MHz, METHANOL-d4) δ |
| | 7.00 − 7.12 (m, 2H), 6.92 (d, J = 8.61 Hz, |
| | 1H), 6.86 (s, 1H), 3.69 (s, 3H), 2.96 (td, J = |
| | 4.01, 7.63 Hz, 1H), 2.37 (s, 3H), 1.98 (ddd, |
| | J = 3.13, 6.26, 9.39 Hz, 1H), 1.20 − 1.34 (m, |
| | 2H); LCMS: ESI [M + H]⁺ = 348.0 |
| 152 | ¹H NMR (400 MHz, CHLOROFORM-d) δ |
| | 7.25 − 7.42 (m, 5H), 6.86 (s, 1H), 6.67 (d, |
| | J = 16.04 Hz, 1H), 6.26 − 6.39 (m, 1H), 5.38 |
| | (br. s., 1H), 4.46 (t, J = 5.87 Hz, 2H), 2.61 |
| | (s, 3H); LCMS: ESI [M + H]⁺ = 350.1 |
| 153 | ¹H NMR (400 MHz, CHLOROFORM-d) δ |
| | 7.28 − 7.35 (m, 2H), 7.10 − 7.22 (m, 3H), |
| | 6.99 − 7.08 (m, 1H), 6.91 (dd, J = 8.22, |
| | 10.56 Hz, 1H), 6.83 (d, J = 8.61 Hz, 1H), |
| | 6.69 (s, 1H), 5.40 (br. s., 1H), 3.03 (br. s., |
| | 1H), 2.46 (s, 3H), 2.03 − 2.12 (m, 1H), |
| | 1.27 − 1.32 (m, 2H); LCMS: ESI [M + H]⁺ = |
| | 410.1 |
| 154 | ¹H NMR (400 MHz, METHANOL-d4) δ |
| | 7.55 − 7.60 (m, 2H), 7.36 (d, J = 5.09 Hz, |
| | 3H), 7.16 − 7.24 (m, 2H), 7.03 − 7.14 (m, |
| | 3H), 6.89 (s, 1H), 3.14 − 3.20 (m, 2H), 2.46 |
| | (s, 3H), 2.40 (t, J = 7.83 Hz, 2H), 1.65 |
| | (quin, J = 7.53 Hz, 2H); LCMS: ESI |
| | [M + H]⁺ = 392.1 |
| 155 | ¹H NMR (400 MHz, CHLOROFORM-d) δ |
| | 7.63 (dd, J = 2.93, 6.46 Hz, 2H), 7.28 − 7.35 |
| | (m, 3H), 7.02 − 7.13 (m, 1H), 6.85 − 6.97 |
| | (m, 2H), 6.66 (s, 1H), 5.31 (br. s., 1H), 2.92 |
| | (d, J = 3.13 Hz, 1H), 2.46 (s, 3H), 1.97 − |
| | 2.11 (m, 1H), 1.13 − 1.30 (m, 2H); LCMS: |
| | ESI [M + H]⁺ = 426.1 |
| 156 | Two tautomers azido/tetrazolo: Major |
| | tautomer ¹H NMR (400 MHz, |
| | CHLOROFORM-d) δ 7.31 (d, J = 7.43 Hz, |
| | 2H), 7.19 − 7.25 (m, 3H), 6.53 (s, 1H), 5.01 |
| | (br. s., 1H), 3.65 (q, J = 6.65 Hz, 2H), 2.76 |
| | (t, J = 7.43 Hz, 2H), 2.52 (s, 3H), 2.04 |
| | (quin, J = 7.24 Hz, 2H); LCMS: ESI |
| | [M + H]⁺ = 325.1 |
| | LCMS: ESI [M + H]⁺ = 325.1 |
| 157 | ¹H NMR (400 MHz, CHLOROFORM-d) δ |
| | 8.49 (td, J = 5.9, 5.4, 2.2 Hz, 2H), 7.15 (q, |
| | J = 5.3, 3.7 Hz, 2H), 6.78 (t, J = 4.1 Hz, 1H), |
| | 5.49 (d, J = 6.3 Hz, 1H), 3.69 (t, J = 6.5 Hz, |
| | 2H), 2.73 (td, J = 7.5, 4.5 Hz, 2H), 2.58 (t, |
| | J = 4.0 Hz, 3H), 2.11 − 1.98 (m, 2H); LCMS: |
| | ESI [M + H]⁺ = 353.1 |
| 158 | ¹H NMR (400 MHz, CHLOROFORM-d) δ |
| | 8.45 (dt, J = 6.3, 2.0 Hz, 2H), 7.55 (dt, J = |
| | 7.8, 2.0 Hz, 1H), 7.22 (dd, J = 7.8, 4.8 Hz, |
| | 1H), 6.82 − 6.77 (m, 1H), 5.58 (d, J = 6.8 |
| | Hz, 1H), 3.69 (q, J = 6.7 Hz, 2H), 2.74 (t, |
| | J = 7.6 Hz, 2H), 2.57 (s, 3H), 2.03 (quin, J = |
| | 7.4 Hz, 2H); LCMS: ESI [M + H]⁺ = 353.1 |
| 159 | ¹H NMR (400 MHz, CHLOROFORM-d) δ |
| | 8.56 (ddd, J = 4.9, 1.9, 1.0 Hz, 1H), 7.67 − |
| | 7.58 (m, 1H), 7.24 − 7.12 (m, 2H), 6.93 (t, |
| | J = 5.3 Hz, 1H), 6.89 (q, J = 1.2 Hz, 1H), |
| | 3.68 (q, J = 6.0 Hz, 2H), 2.96 (t, J = 6.8 Hz, |
| | 2H), 2.59 (d, J = 1.1 Hz, 3H), 2.18 − 2.10 |
| | (m, 2H); LCMS: ESI [M + H]⁺ = 353.1 |
| 160 | ¹H NMR (400 MHz, CHLOROFORM-d) o |
| | 7.30 (dd, J = 4.7, 1.4 Hz, 8H), 7.20 (tdd, J = |
| | 7.7, 3.3, 2.0 Hz, 2H), 6.54 − 6.48 (m, 1H), |
| | 5.09 (d, J = 6.8 Hz, 1H), 4.05 (t, J = 7.7 Hz, |
| | 1H), 3.69 (q, J = 6.6 Hz, 2H), 2.57 (d, J = |
| | 1.1 Hz, 3H), 2.48 (q, J = 7.1 Hz, 2H); |
| | LCMS: ESI [M + H]⁺ = 428.1 |
| 161 | ¹H NMR (400 MHz, CHLOROFORM-d) δ |
| | 7.25 − 7.14 (m, 2H), 6.97 − 6.84 (m, 2H), |
| | 6.70 − 6.66 (m, 1H), 5.40 (s, 1H), 3.83 (s, |
| | 3H), 3.65 (q, J = 6.4 Hz, 2H), 2.76 (t, J = |
| | 7.2 Hz, 2H), 2.59 (d, J = 1.2 Hz, 3H), 1.99 |
| | (quin, J = 6.9 Hz, 2H); LCMS: ESI |
| | [M + H]⁺ = 382.0 |
| 162 | ¹H NMR (400 MHz, CHLOROFORM-d) δ |
| | 7.47 − 7.39 (m, 2H), 7.38 − 7.28 (m, 3H), |
| | 6.89 (t, J = 1.3 Hz, 1H), 5.45 (d, J = 5.3 Hz, |
| | 1H), 4.69 (d, J = 5.1 Hz, 2H), 2.61 (d, J = |
| | 1.3 Hz, 3H); LCMS: ESI [M + H]⁺ = 348.1 |
| 163 | ¹H NMR (400 MHz, METHANOL-d4) δ |
| | 7.24 (d, J = 4.4 Hz, 3H), 7.19 − 7.12 (m, |
| | 2H), 4.19 (tdd, J = 7.4, 5.9, 4.2 Hz, 1H), |
| | 3.76 (dd, J = 13.8, 4.2 Hz, 1H), 3.53 (dd, |
| | J = 13.7, 7.4 Hz, 1H), 2.90 − 2.75 (m, 2H), |
| | 2.60 (d, J = 1.3 Hz, 3H), 1.30 (d, J = 15.4 |
| | Hz, 1H); LCMS: ESI [M + H]⁺ = 368.1 |
| 164 | ¹H NMR (400 MHz, CHLOROFORM-d) δ |
| | 6.73 (t, J = 2.0 Hz, 2H), 6.65 − 6.59 (m, |
| | 1H), 6.24 (t, J = 2.0 Hz, 2H), 4.72 (t, J = |
| | 5.6 Hz, 1H), 4.07 (t, J = 6.4 Hz, 2H), 3.75 − |
| | 3.66 (m, 2H), 2.58 (d, J = 1.4 Hz, 3H), 2.20 − |
| | 2.11 (m, 2H); LCMS: ESI [M + H]⁺ = |
| | 341.1 |
| 165 | ¹H NMR (400 MHz, CHLOROFORM-d) δ |
| | 7.06 (t, J = 7.7 Hz, 1H), 6.99 (d, J = 7.3 Hz, |
| | 1H), 6.63 (q, J = 7.7, 7.3 Hz, 2H), 6.48 (s, |
| | 1H), 5.57 (t, J = 5.6 Hz, 1H), 3.75 (q, J = |
| | 6.4 Hz, 2H), 3.43 (t, J = 6.6 Hz, 2H), 3.28 |
| | (t, J = 5.6 Hz, 2H), 2.75 (t, J = 6.4 Hz, 2H), |
| | 2.54 (d, J = 1.6 Hz, 3H), 2.02 (quin, J = 6.5 |
| | Hz, 2H), 1.93 (quin, J = 6.3 Hz, 2H); |
| | LCMS: ESI [M + H]⁺ = 407.0 |
| 166 | ¹H NMR (400 MHz, CHLOROFORM-d) δ |
| | 7.27 (qd, J = 7.7, 3.5 Hz, 2H), 7.20 (dd, J = |
| | 7.3, 4.4 Hz, 3H), 6.65 − 6.52 (m, 1H), 5.12 |
| | (q, J = 5.4 Hz, 1H), 3.68 (dq, J = 12.4, 6.4, |
| | 5.5 Hz, 2H), 2.74 (td, J = 7.4, 4.2 Hz, 2H), |
| | 2.55 (d, J = 4.4 Hz, 3H), 2.03 (td, J = 7.2, |
| | 4.5 Hz, 2H); LCMS: ESI [M + H]⁺ = 352.1 |
| 167 | ¹H NMR (400 MHz, CHLOROFORM-d) δ |
| | 7.34 (t, J = 7.5 Hz, 2H), 7.25 (t, J = 6.6 Hz, |
| | 3H), 6.86 (s, 1H), 5.76 (s, 1H), 3.12 (dq, J = |
| | 7.0, 3.3 Hz, 1H), 2.46 (s, 3H), 2.21 (ddd, |
| | J = 9.7, 6.4, 3.3 Hz, 1H), 1.48 (q, J = 6.6 |
| | Hz, 1H), 1.34 (dt, J = 9.6, 4.9 Hz, 1H); |
| | LCMS: ESI [M + H]⁺ = 350.1 |
| 168 | ¹H NMR (400 MHz, CHLOROFORM-d) δ |
| | 7.34 (t, J = 7.5 Hz, 2H), 7.25 (t, J = 6.5 Hz, |
| | 3H), 6.86 (d, J = 1.4 Hz, 1H), 5.76 (s, 1H), |
| | 3.16 − 3.07 (m, 1H), 2.46 (s, 3H), 2.21 (ddd, |
| | J = 9.6, 6.3, 3.2 Hz, 1H), 1.48 (q, J = 6.6 |
| | Hz, 1H), 1.34 (dt, J = 10.0, 5.0 Hz, 1H); |
| | LCMS: ESI [M + H]⁺ = 350.1 |
| 169 | ¹H NMR (400 MHz, CHLOROFORM-d) δ |
| | 7.23 (t, J = 7.2 Hz, 2H), 7.19 − 7.14 (m, |
| | 1H), 7.14 − 7.09 (m, 2H), 6.76 (d, J = 1.4 |
| | Hz, 1H), 5.32 (tdd, J = 10.4, 7.2, 3.1 Hz, |
| | 1H), 5.00 (d, J = 9.8 Hz, 1H), 2.78 (t, J = |
| | 7.5 Hz, 2H), 2.63 (d, J = 1.3 Hz, 3H), 2.32 |
| | (dtd, J = 15.5, 7.9, 3.3 Hz, 1H), 2.04 (ddt, |
| | J = 14.0, 10.5, 7.0 Hz, 1H); LCMS: ESI |
| | M + H]⁺ = 420.1 |
| 170 | ¹H NMR (400 MHz, CHLOROFORM-d) δ |
| | 7.29 (d, J = 3.2 Hz, 8H), 7.20 (ddd, J = 8.8, |
| | 5.9, 3.1 Hz, 2H), 6.46 (t, J = 1.4 Hz, 1H), |
| | 4.86 − 4.79 (m, 1H), 4.05 (t, J = 7.7 Hz, |
| | 1H), 3.64 (q, J = 6.6 Hz, 2H), 2.82 (q, J = |
| | 7.6 Hz, 2H), 2.51 (d, J = 1.5 Hz, 3H), 2.46 |
| | (q, J = 7.3 Hz, 2H), 1.33 (t, J = 7.6 Hz, 3H); |
| | LCMS: ESI [M + H]⁺ = 388.1 |
| 171 | ¹H NMR (400 MHz, CHLOROFORM-d) δ |
| | 7.41 − 7.24 (m, 5H), 6.79 (s, 1H), 6.15 − |
| | 6.05 (m, 1H), 4.85 (t, J = 6.2 Hz, 1H), 4.06 |
| | (dt, J = 12.7, 6.3 Hz, 1H), 3.63 (dt, J = 14.5, |
| | 4.6 Hz, 1H), 3.26 (d, J = 10.7 Hz, 1H), 2.59 |
| | (s, 3H), 2.06 (qd, J = 5.9, 2.2 Hz, 2H); |
| | LCMS: ESI [M + H]⁺ = 368.0 |
| 172 | ¹H NMR (400 MHz, CHLOROFORM-d) δ |
| | 7.26 (d, J = 15.5 Hz, 2H), 6.77 (dd, J = |
| | 15.0, 7.7 Hz, 3H), 6.54 (s, 1H), 5.56 (d, J = |
| | 5.8 Hz, 1H), 3.72 (q, J = 6.3 Hz, 2H), 3.48 |
| | (t, J = 6.6 Hz, 2H), 2.94 (s, 3H), 2.55 (s, |
| | 3H), 2.01 (quin, J = 6.8 Hz, 2H); LCMS: |
| | ESI [M + H]⁺ = 381.0 |
| 173 | ¹H NMR (400 MHz, CHLOROFORM-d) δ |
| | 7.23 − 7.17 (m, 2H), 6.74 (t, J = 7.3 Hz, |
| | 1H), 6.71 − 6.64 (m, 3H), 5.62 (s, 1H), 3.81 |
| | (q, J = 6.1 Hz, 2H), 3.33 − 3.25 (m, 2H), |
| | 2.57 (d, J = 1.6 Hz, 3H), 1.98 (quin, J = 6.1 |
| | Hz, 2H); LCMS: ESI [M + H]⁺ = 367.0 |
| 175 | ¹H NMR (400 MHz, CHLOROFORM-d) δ |
| | 7.31 − 7.38 (m, 2H), 7.25 (d, J = 10.17 Hz, |
| | 2H), 7.13 − 7.22 (m, 3H), 3.18 (br. s., 1H), |
| | 2.91 (q, J = 7.43 Hz, 2H), 2.22 − 2.30 (m, |
| | J = 6.26 Hz, 1H), 1.41 − 1.52 (m, J = 6.46, |
| | 6.46 Hz, 2H), 1.38 (t, J = 7.43 Hz, 3H); |
| | LCMS: ESI [M + H]⁺ = 296.1 |
| 176 | ¹H NMR (400 MHz, CHLOROFORM-d) δ |
| | 7.28 (d, J = 7.04 Hz, 2H), 7.17 − 7.23 (m, |
| | 3H), 6.82 (s, 1H), 5.79 (br. s., 1H), 3.03 − |
| | 3.10 (m, J = 3.13 Hz, 1H), 2.40 (s, 3H), |
| | 2.12 − 2.19 (m, 1H), 1.43 (q, J = 6.52 Hz, |
| | 1H), 1.30 (td, J = 4.94, 9.29 Hz, 1H); |
| | LCMS: ESI [M + H]⁺ = 350.1 |
| 177 | ¹H NMR (400 MHz, CHLOROFORM-d) δ |
| | 7.66 (d, J = 7.9 Hz, 1H), 7.37 (d, J = 8.2 |
| | Hz, 1H), 7.22 (t, J = 7.8 Hz, 1H), 7.17 − |
| | 7.09 (m, 2H), 6.53 (d, J = 3.3 Hz, 1H), 6.20 |
| | (s, 1H), 4.70 (s, 1H), 4.31 (t, J = 6.0 Hz, |
| | 2H), 3.67 (q, J = 6.0 Hz, 2H), 2.54 (s, 3H), |
| | 2.26 (q, J = 6.2 Hz, 2H), 1.57 (s, 1H); |
| | LCMS: ESI [M + H]⁺ = 391.1 |
| 178 | ¹H NMR (400 MHz, CHLOROFORM-d) δ |
| | 7.14 − 7.08 (m, 2H), 6.74 − 6.68 (m, 2H), |
| | 6.46 (s, 1H), 5.05 (s, 1H), 3.70 (q, J = 6.0 |
| | Hz, 2H), 2.94 (d, J = 2.5 Hz, 6H), 2.70 (t, |
| | J = 7.1 Hz, 2H), 2.57 − 2.52 (m, 3H), 2.01 |
| | (quin, J = 7.0 Hz, 2H); LCMS: ESI |
| | [M + H]⁺ = 395.1 |
| 179 | ¹H NMR (400 MHz, CHLOROFORM-d) δ |
| | 7.48 − 7.36 (m, 4H), 6.74 − 6.68 (m, 1H), |
| | 5.18 (s, 1H), 3.72 (q, J = 6.6 Hz, 2H), 2.81 |
| | (t, J = 7.7 Hz, 2H), 2.59 (d, J = 3.1 Hz, 3H), |
| | 2.07 (quin, J = 7.3 Hz, 2H); LCMS: ESI |
| | [M + H]⁺ = 420.0 |
| 180 | ¹H NMR (400 MHz, CHLOROFORM-d) δ |
| | 8.13 (d, J = 8.4 Hz, 1H), 8.08 (d, J = 8.6 |
| | Hz, 1H), 7.83 (d, J = 8.2 Hz, 1H), 7.76 (t, J = |
| | 7.9 Hz, 1H), 7.55 (t, J = 7.7 Hz, 1H), 7.35 |
| | (d, J = 7.5 Hz, 1H), 7.20 (s, 1H), 6.86 (s, |
| | 1H), 4.17 (d, J = 6.2 Hz, 2H), 3.34 (t, J = |
| | 6.2 Hz, 2H), 2.59 (s, 3H); LCMS: ESI |
| | [M + H]⁺ = 389.1 |
| 181 | ¹H NMR (400 MHz, CHLOROFORM-d) δ |
| | 7.30 (s, 2H), 7.02 (t, J = 8.6 Hz, 2H), 6.85 |
| | (s, 1H), 5.66 (s, 1H), 3.05 (s, 1H), 2.52 (s, |
| | 3H), 2.18 (s, 1H), 1.41 (d, J = 7.4 Hz, 1H), |
| | 1.31 (s, 1H); LCMS: ESI [M + H]⁺ = 368.1 |
| 182 | ¹H NMR (400 MHz, DMSO-d6) 8 9.95 (s, |
| | 1H), 8.47 (s, 1H), 7.58 (d, J = 7.9 Hz, 2H), |
| | 7.41 (s, 1H), 7.28 (t, J = 7.9 Hz, 2H), 7.02 |
| | (t, J = 7.3 Hz, 1H), 3.78 (d, J = 6.6 Hz, 2H), |
| | 2.70 (d, J = 6.9 Hz, 2H), 2.57 (s, 3H); |
| | LCMS: ESI [M + H]⁺ = 381.1 |
| 183 | ¹H NMR (400 MHz, CHLOROFORM-d) δ |
| | 6.71 − 6.64 (m, 3H), 6.23 − 6.17 (m, 2H), |
| | 5.21 (s, 1H), 4.20 (q, J = 4.4, 3.2 Hz, 2H), |
| | 3.97 (q, J = 5.7 Hz, 2H), 2.58 (d, J = 2.9 |
| | Hz, 3H); LCMS: ESI [M + H]⁺ = 327.0 |
| 184 | ¹H NMR (400 MHz, METHANOL-d₄) δ |
| | 7.12 (s, 1H), 7.02 (d, J = 7.9 Hz, 2H), 6.68 |
| | (d, J = 7.5 Hz, 2H), 3.60 (t, J = 7.4 Hz, 2H), |
| | 2.60 (d, J = 12.7 Hz, 5H), 1.96 (quin, J = |
| | 7.7 Hz, 2H); LCMS: ESI [M + H]⁺ = 368.3 |
| 185 | ¹H NMR (400 MHz, CHLOROFORM-d) δ |
| | 7.16 (tt, J = 5.2, 2.6 Hz, 2H), 6.97 (td, J = |
| | 8.8, 2.7 Hz, 2H), 6.67 (s, 1H), 5.16 (s, 1H), |
| | 3.69 (q, J = 6.3 Hz, 2H), 2.77 − 2.68 (m, |
| | 2H), 2.58 (d, J = 3.1 Hz, 3H), 2.08 − 1.96 |
| | (m, 2H); LCMS: ESI [M + H]⁺ = 370.3 |
| 186 | ¹H NMR (400 MHz, CHLOROFORM-d) δ |
| | 7.31 (dd, J = 13.0, 7.0 Hz, 3H), 7.21 (d, J = |
| | 7.2 Hz, 2H), 6.48 (s, 1H), 5.01 (d, J = 7.0 |
| | Hz, 1H), 3.61 (dd, J = 6.5, 2.3 Hz, 2H), |
| | 2.65 (d, J = 2.4 Hz, 2H), 2.58 (s, 3H), 1.02 |
| | (d, J = 2.4 Hz, 6H); LCMS: ESI [M + H]⁺ = |
| | 380.0 |
| 187 | ¹H NMR (400 MHz, CHLOROFORM-d) δ |
| | 7.32 (d, J = 7.5 Hz, 2H), 7.25 (d, J = 8.0 |
| | Hz, 3H), 6.47 (s, 1H), 4.99 (s, 1H), 3.69 |
| | (dd, J = 13.9, 6.9 Hz, 1H), 3.58 − 3.48 (m, |
| | 1H), 2.85 (q, J = 7.3 Hz, 1H), 2.56 (s, 3H), |
| | 2.01 (dq, J = 14.2, 7.7, 6.5 Hz, 2H), 1.33 |
| | (dd, J = 6.9, 2.5 Hz, 3H); LCMS: ESI |
| | [M + H]⁺ = 366.0 |
| 188 | ¹H NMR (400 MHz, CHLOROFORM-d) δ |
| | 7.17 − 7.31 (m, 2H), 6.99 − 7.12 (m, 2H), |
| | 6.76 (br. s., 1H), 5.31 (br. s., 1H), 3.70 (d, |
| | J = 5.87 Hz, 2H), 2.74 − 2.85 (m, 2H), 2.60 |
| | (br. s., 3H), 2.04 (t, J = 6.65 Hz, 2H); |
| | LCMS: ESI [M + H]⁺ = 370.0 |
| 189 | ¹H NMR (400 MHz, CHLOROFORM-d) δ |
| | 7.19 (q, J = 8.0 Hz, 2H), 6.94 − 6.83 (m, |
| | 2H), 6.62 (s, 1H), 5.21 (s, 1H), 3.82 (d, J = |
| | 2.8 Hz, 3H), 3.62 (q, J = 7.1, 6.6 Hz, 2H), |
| | 2.88 − 2.77 (m, 2H), 2.77 − 2.71 (m, 2H), |
| | 2.52 (s, 3H), 1.97 (t, J = 7.3 Hz, 2H), 1.34 |
| | (td, J = 7.5, 2.6 Hz, 3H); LCMS: ESI |
| | [M + H]⁺ = 342.2 |
| 190 | ¹H NMR (400 MHz, CHLOROFORM-d) δ |
| | 7.45 (d, J = 6.8 Hz, 2H), 7.39 (d, J = 3.9 |
| | Hz, 2H), 6.64 (s, 1H), 5.18 (s, 1H), 3.68 (q, |
| | J = 6.4 Hz, 2H), 2.82 (quin, J = 7.4 Hz, |
| | 4H), 2.52 (s, 3H), 2.06 (q, J = 7.5 Hz, 2H), |
| | 1.33 (t, J = 7.6 Hz, 3H); LCMS: ESI |
| | [M + H]⁺ = 380.2 |
| 191 | ¹H NMR (400 MHz, CHLOROFORM-d) δ |
| | 7.16 (dd, J = 8.4, 5.1 Hz, 2H), 6.97 (t, J = |
| | 8.7 Hz, 2H), 6.60 (s, 1H), 4.92 (s, 1H), 3.65 |
| | (q, J = 6.5 Hz, 2H), 2.82 (q, J = 7.7 Hz, |
| | 2H), 2.71 (t, J = 7.7 Hz, 2H), 2.52 (s, 3H), |
| | 2.00 (q, J = 7.4 Hz, 2H), 1.34 (t, J = 7.7 Hz, |
| | 3H); LCMS: ESI [M + H]⁺ = 330.1 |
| 192 | ¹H NMR (400 MHz, CHLOROFORM-d) δ |
| | 7.04 (t, J = 8.0 Hz, 1H), 6.97 (d, J = 7.4 Hz, |
| | 1H), 6.60 (q, J = 8.1, 7.4 Hz, 2H), 6.51 (s, |
| | 1H), 5.25 (s, 1H), 3.70 (q, J = 6.4 Hz, 2H), |
| | 3.41 (t, J = 7.0 Hz, 2H), 3.27 (t, J = 5.5 Hz, |
| | 2H), 2.83 (q, J = 7.7 Hz, 2H), 2.75 (t, J = |
| | 6.4 Hz, 2H), 2.49 (s, 3H), 1.97 (dt, J = 25.0, |
| | 6.4 Hz, 4H), 1.35 (t, J = 7.7 Hz, 3H); |
| | LCMS: ESI [M + H]⁺ = 367.2 |
| 193 | ¹H NMR (400 MHz, CHLOROFORM-d) δ |
| | 7.43 (dd, J = 6.9, 3.2 Hz, 2H), 7.30 (dt, J = |
| | 5.8, 2.7 Hz, 3H), 6.77 (s, 1H), 5.17 (s, 1H), |
| | 4.69 − 4.63 (m, 2H), 2.87 (q, J = 7.4 Hz, |
| | 2H), 2.54 (t, J = 1.8 Hz, 3H), 1.37 (t, J = |
| | 7.6 Hz, 3H); LCMS: ESI [M + H]⁺ = 308.1 |
| 194 | ¹H NMR (400 MHz, CHLOROFORM-d) δ |
| | 7.32 (t, J = 7.6 Hz, 2H), 7.28 − 7.22 (m, |
| | 3H), 6.72 − 6.67 (m, 1H), 5.38 (t, J = 5.6 |
| | Hz, 1H), 4.12 (pd, J = 5.3, 3.4, 2.5 Hz, 1H), |
| | 3.75 (ddd, J = 14.3, 6.2, 2.5 Hz, 1H), 3.55 |
| | (dd, J = 13.2, 7.4 Hz, 1H), 2.95 (dd, J = |
| | 13.7, 6.2 Hz, 1H), 2.89 − 2.75 (m, 3H), 2.56 − |
| | 2.51 (m, 3H), 1.39 − 1.30 (m, 3H); LCMS: |
| | ESI [M + H]⁺ = 328.2 |
| 195 | ¹H NMR (400 MHz, CHLOROFORM-d) δ |
| | 7.25 (ddd, J = 8.8, 4.4, 2.2 Hz, 2H), 6.80 − |
| | 6.69 (m, 3H), 6.53 (q, J = 1.6 Hz, 1H), 5.14 |
| | (s, 1H), 3.68 (qd, J = 6.6, 2.4 Hz, 2H), 3.47 |
| | (td, J = 6.8, 2.5 Hz, 2H), 2.94 (d, J = 2.6 |
| | Hz, 3H), 2.83 (qd, J = 7.6, 2.6 Hz, 2H), |
| | 2.51 (q, J = 1.4 Hz, 3H), 2.00 (qd, J = 6.9, |
| | 2.5 Hz, 2H), 1.35 (td, J = 7.6, 2.5 Hz, 3H); |
| | LCMS: ESI [M + H]⁺ = 341.2 |
| 196 | ¹H NMR (400 MHz, CHLOROFORM-d) δ |
| | 7.66 (dt, J = 7.9, 1.5 Hz, 1H), 7.40 − 7.33 |
| | (m, 1H), 7.24 − 7.19 (m, 1H), 7.19 − 7.09 |
| | (m, 2H), 6.56 − 6.50 (m, 1H), 6.33 (dt, J = |
| | 2.4, 1.2 Hz, 1H), 4.63 (t, J = 5.8 Hz, 1H), |
| | 4.29 (td, J = 6.6, 2.2 Hz, 2H), 3.63 (qd, J = |
| | 6.4, 2.2 Hz, 2H), 2.82 (qd, J = 7.6, 2.2 Hz, |
| | 2H), 2.53 − 2.48 (m, 3H), 2.26 (pd, J = 6.4, |
| | 2.1 Hz, 2H), 1.38 − 1.29 (m, 3H); LCMS: |
| | ESI [M + H]⁺ = 351.2 |
| 197 | ¹H NMR (400 MHz, CHLOROFORM-d) δ |
| | 7.57 (t, J = 2.2 Hz, 1H), 7.43 (d, J = 2.4 Hz, |
| | 1H), 6.72 (q, J = 1.6 Hz, 1H), 6.28 (dt, J = |
| | 3.9, 2.0 Hz, 1H), 5.68 (s, 1H), 4.29 (td, J = |
| | 6.5, 2.5 Hz, 2H), 3.63 (qd, J = 6.2, 2.4 Hz, |
| | 2H), 2.82 (qd, J = 7.7, 2.5 Hz, 2H), 2.56 − |
| | 2.51 (m, 3H), 2.21 (dtd, J = 8.9, 6.8, 4.2 |
| | Hz, 2H), 1.34 (td, J = 7.7, 2.5 Hz, 3H); |
| | LCMS: ESI [M + H]⁺ = 302.2 |
| 198 | ¹H NMR (400 MHz, CHLOROFORM-d) δ |
| | 8.03 (dd, J = 7.1, 2.6 Hz, 1H), 7.87 (dt, J = |
| | 8.3, 2.2 Hz, 1H), 7.73 (d, J = 7.7 Hz, 1H), |
| | 7.54 − 7.44 (m, 2H), 7.44 − 7.34 (m, 2H), |
| | 6.51 (d, J = 1.5 Hz, 1H), 4.87 (s, 1H), 3.74 |
| | (q, J = 6.9 Hz, 2H), 3.26 − 3.17 (m, 2H), |
| | 2.83 (qd, J = 7.6, 2.1 Hz, 2H), 2.54 − 2.49 |
| | (m, 3H), 2.18 (h, J = 5.9, 5.2 Hz, 2H), 1.38 − |
| | 1.26 (m, 3H); LCMS: ESI [M + H]⁺ = |
| | 362.2 |
| 199 | ¹H NMR (400 MHz, CHLOROFORM-d) δ |
| | 7.41 (dd, J = 8.5, 2.1 Hz, 2H), 7.13 − 7.05 |
| | (m, 2H), 6.56 (q, J = 1.4 Hz, 1H), 4.86 (s, |
| | 1H), 3.65 (qd, J = 6.8, 2.0 Hz, 2H), 2.82 |
| | (qd, J = 7.6, 2.1 Hz, 2H), 2.70 (td, J = 7.5, |
| | 2.0 Hz, 2H), 2.53 (q, J = 1.3 Hz, 3H), 2.00 |
| | (pd, J = 7.1, 2.0 Hz, 2H), 1.34 (td, J = 7.6, |
| | 2.0 Hz, 3H); LCMS: ESI [M + H]⁺ = 390.1, |
| | 392.1 |
| 200 | ¹H NMR (400 MHz, CHLOROFORM-d) δ |
| | 7.25 − 7.14 (m, 2H), 7.12 − 6.98 (m, 2H), |
| | 6.66 (q, J = 1.5 Hz, 1H), 4.98 (s, 1H), 3.66 |
| | (qd, J = 6.7, 2.3 Hz, 2H), 2.88 − 2.73 (m, |
| | 4H), 2.53 (q, J = 1.3 Hz, 3H), 2.01 (pd, J = |
| | 7.0, 2.3 Hz, 2H), 1.34 (td, J = 7.6, 2.3 Hz, |
| | 3H); LCMS: ESI [M + H]⁺ = 330.2 |
| 201 | ¹H NMR (400 MHz, CHLOROFORM-d) δ |
| | 7.32 (ddd, J = 9.0, 7.4, 2.3 Hz, 2H), 7.18 |
| | (dd, J = 8.6, 2.3 Hz, 2H), 7.13 − 7.04 (m, |
| | 1H), 7.03 − 6.92 (m, 4H), 6.65 − 6.59 (m, |
| | 1H), 4.94 (s, 1H), 3.67 (dt, J = 9.1, 5.9 Hz, |
| | 2H), 2.83 (qd, J = 7.6, 2.2 Hz, 2H), 2.73 |
| | (td, J = 7.5, 2.2 Hz, 2H), 2.58 − 2.46 (m, |
| | 3H), 2.02 (pd, J = 7.2, 2.1 Hz, 2H), 1.35 |
| | (td, J = 7.6, 2.2 Hz, 3H); LCMS: ESI |
| | [M + H]⁺ = 404.2 |
| 202 | ¹H NMR (400 MHz, CHLOROFORM-d) δ |
| | 7.37 (s, 1H), 7.35 − 7.30 (m, 1H), 7.19 − |
| | 7.09 (m, 2H), 6.62 (t, J = 1.4 Hz, 1H), 4.90 |
| | (d, J = 6.8 Hz, 1H), 3.66 (q, J = 6.6, 6.0 Hz, |
| | 2H), 2.83 (qd, J = 7.6, 1.3 Hz, 2H), 2.71 (t, |
| | J = 7.6 Hz, 2H), 2.53 (d, J = 1.4 Hz, 3H), |
| | 2.00 (quin, J = 7.0 Hz, 2H), 1.34 (td, J = |
| | 7.6, 1.3 Hz, 3H); LCMS: ESI [M + H]⁺ = |
| | 390.1, 392.1 |
| 203 | ¹H NMR (400 MHz, CHLOROFORM-d) δ |
| | 7.33 (dd, J = 8.3, 2.2 Hz, 2H), 7.16 (dd, J = |
| | 8.3, 2.1 Hz, 2H), 6.57 (q, J = 1.4 Hz, 1H), |
| | 4.89 (s, 1H), 3.72 − 3.62 (m, 2H), 2.82 (qd, |
| | J = 7.6, 2.2 Hz, 2H), 2.73 (td, J = 7.6, 2.1 |
| | Hz, 2H), 2.54 − 2.49 (m, 3H), 2.02 (dd, J = |
| | 10.2, 4.4 Hz, 2H), 1.38 − 1.29 (m, 12H); |
| | LCMS: ESI [M + H]⁺ = 368.2 |
| 204 | ¹H NMR (400 MHz, CHLOROFORM-d) δ |
| | 7.45 − 7.36 (m, 2H), 7.14 − 7.06 (m, 2H), |
| | 6.62 (q, J = 1.5 Hz, 1H), 5.08 (s, 1H), 3.70 |
| | (td, J = 6.5, 6.0, 2.7 Hz, 2H), 2.71 (td, J = |
| | 7.3, 2.7 Hz, 2H), 2.59 (dd, J = 2.9, 1.5 Hz, |
| | 3H), 2.02 (pd, J = 6.9, 2.5 Hz, 2H); LCMS: |
| | ESI [M + H]⁺ = 430.0, 432.0 |
| 205 | ¹H NMR (400 MHz, CHLOROFORM-d) δ |
| | 8.03 (d, J = 7.7 Hz, 1H), 7.88 − 7.82 (m, |
| | 1H), 7.73 (d, J = 7.4 Hz, 1H), 7.55 − 7.44 |
| | (m, 2H), 7.44 − 7.34 (m, 2H), 6.50 (q, J = |
| | 1.3 Hz, 1H), 5.07 (s, 1H), 3.78 (q, J = 6.4 |
| | Hz, 2H), 3.23 (t, J = 7.5 Hz, 2H), 2.56 (q, J = |
| | 1.2 Hz, 3H), 2.20 (quin, J = 7.1 Hz, 2H); |
| | LCMS: ESI [M + H]⁺ = 402.1 |
| 206 | ¹H NMR (400 MHz, CHLOROFORM-d) δ |
| | 7.37 − 7.29 (m, 2H), 7.20 − 7.12 (m, 2H), |
| | 6.63 (q, J = 1.3 Hz, 1H), 5.12 (s, 1H), 3.71 |
| | (td, J = 6.8, 5.7 Hz, 2H), 2.74 (t, J = 7.4 Hz, |
| | 2H), 2.57 (d, J = 1.2 Hz, 3H), 2.05 (quin, J = |
| | 7.1 Hz, 2H), 1.32 (s, 9H); LCMS: ESI |
| | [M + H]⁺ = 408.0 |
| 207 | ¹H NMR (400 MHz, METHANOL-d4) δ |
| | 6.61 (s, 1H), 5.97 − 6.04 (m, 1H), 5.81 (d, J = |
| | 8.31 Hz, 2H), 5.55 (d, J = 8.19 Hz, 2H), |
| | 5.49 − 5.53 (m, 1H), 5.46 (d, J = 1.35 Hz, |
| | 1H), 2.10 (t, J = 6.54 Hz, 2H), 1.12 (t, J = |
| | 6.97 Hz, 2H), 1.01 (s, 3H), 0.13 − 0.25 (m, J = |
| | 3.42, 3.42 Hz, 4H); LCMS: ESI [M + |
| | H]⁺ = 444.1 |
| 208 | ¹H NMR (399 MHz, METHANOL-d4) δ |
| | 8.33 (s, 1H), 8.26 (s, 1H), 7.53 − 7.60 (m, |
| | 1H), 7.26 (t, J = 7.79 Hz, 2H), 7.06 (s, 2H), |
| | 6.98 (d, J = 8.17 Hz, 2H), 6.90 (t, J = 7.20 |
| | Hz, 1H), 3.77 (t, J = 6.42 Hz, 2H), 3.33 − |
| | 3.40 (m, 4H), 3.23 − 3.29 (m, J = 4.67 Hz, |
| | 4H), 3.12 − 3.22 (m, 2H), 2.56 (s, 3H), 2.12 − |
| | 2.24 (m, 2H); LCMS: ESI [M + H]⁺ = |
| | 434.2 |
| 209 | ¹H NMR (399 MHz, METHANOL-d4) δ |
| | 8.29 (s, 1H), 8.21 − 8.27 (m, 1H), 7.53 − |
| | 7.60 (m, 1H), 7.23 (t, J = 7.98 Hz, 2H), |
| | 7.04 (s, 2H), 6.94 (d, J = 7.79 Hz, 2H), 6.87 |
| | (t, J = 7.40 Hz, 1H), 4.00 (t, J = 6.23 Hz, |
| | 2H), 3.25 − 3.35 (m, 10H), 2.54 (s, 3H); |
| | LCMS: ESI [M + H]⁺ = 420.2 |
| 210 | ¹H NMR (399 MHz, METHANOL-d4) δ |
| | 8.19 − 8.29 (m, 1H), 7.51 − 7.59 (m, 1H), |
| | 7.13 − 7.26 (m, 2H), 6.98 − 7.09 (m, 2H), |
| | 6.79 − 6.90 (m, 3H), 3.94 (t, J = 6.62 Hz, |
| | 2H), 3.67 − 3.75 (m, 2H), 3.59 − 3.66 (m, |
| | 2H), 2.97 − 3.05 (m, 2H), 2.89 − 2.96 (m, |
| | 2H), 2.86 (t, J = 6.62 Hz, 2H), 2.52 (s, 3H); |
| | LCMS: ESI [M + H]⁺ = 448.2 |
| 211 | ¹H NMR (399 MHz, METHANOL-d4) δ |
| | 8.07 (s, 1H), 7.54 (s, 1H), 7.12 − 7.26 (m, |
| | 3H), 6.98 (s, 1H), 6.92 (s, 1H), 3.62 (t, J = |
| | 7.01 Hz, 2H), 2.71 (t, J = 7.20 Hz, 2H), |
| | 2.52 (s, 3H), 1.92 − 2.07 (m, 2H); LCMS: |
| | ESI [M + H]⁺ = 446.0, 448.0 |
| 212 | ¹H NMR (399 MHz, METHANOL-d4) δ |
| | 8.14 (s, 1H), 7.57 (s, 1H), 7.29 (t, J = 7.98 |
| | Hz, 1H), 7.10 (d, J = 10.51 Hz, 1H), 6.97 − |
| | 7.05 (m, 2H), 6.95 (s, 1H), 3.66 (t, J = 7.01 |
| | Hz, 2H), 2.74 (t, J = 7.40 Hz, 2H), 2.55 (s, |
| | 3H), 1.97 − 2.10 (m, 2H); LCMS: ESI |
| | [M + H]⁺ = 402.1 |
| 213 | ¹H NMR (400 MHz, CHLOROFORM-d) δ |
| | 8.20 (br. s., 1H), 7.47 (br. s., 1H), 7.22 − |
| | 7.27 (m, J = 6.62 Hz, 2H), 7.08 (br. s., 1H), |
| | 6.86 (d, J = 7.01 Hz, 2H), 6.73 (br. s., 1H), |
| | 5.34 (br. s., 1H), 4.12 (br. s., 2H), 3.84 − |
| | 3.94 (m, 2H), 2.57 (s, 3H), 2.23 (br. s., 2H); |
| | LCMS: ESI [M + H]⁺ = 400.1 |
| 214 | ¹H NMR (399 MHz, CHLOROFORM-d) δ |
| | 8.10 (s, 1H), 7.47 (t, J = 1.56 Hz, 1H), 7.37 − |
| | 7.44 (m, 2H), 7.03 − 7.10 (m, J = 8.17 Hz, |
| | 2H), 6.92 − 6.98 (m, 1H), 6.85 (s, 1H), 3.73 − |
| | 3.82 (m, 2H), 3.35 (s, 3H), 2.67 (t, J = |
| | 7.20 Hz, 2H), 2.50 (d, J = 1.17 Hz, 3H), |
| | 2.04 (quin, J = 7.49 Hz, 2H); LCMS: ESI |
| | [M + H]⁺ = 442.0 and 440.0 |
| 215 | LCMS: ESI [M + H]⁺ = 396.0, 398.0 |
| 216 | LCMS: ESI [M + H]⁺ = 397.0, 399.0 |
| 217 | LCMS: ESI [M + H]⁺ = 428.0, 430.0 |
| 218 | ¹H NMR (400 MHz, CHLOROFORM-d) δ |
| | 7.30 − 7.35 (m, 2H), 7.21 − 7.26 (m, 4H), |
| | 6.88 (d, J = 6.26 Hz, 1H), 5.19 (br. s., 1H), |
| | 3.70 (q, J = 6.52 Hz, 2H), 2.79 (t, J = 7.24 |
| | Hz, 2H), 2.07 (quin, J = 7.14 Hz, 2H); |
| | LCMS: ESI [M + H]⁺ = 304.1 |
| 219 | ¹H NMR (400 MHz, CHLOROFORM-d) δ |
| | 7.23 (d, J = 5.87 Hz, 1H), 7.15 (d, J = 8.22 |
| | Hz, 2H), 6.89 (d, J = 5.87 Hz, 1H), 6.86 (d, |
| | J = 8.22 Hz, 2H), 3.80 (s, 3H), 3.69 (q, J = |
| | 6.65 Hz, 2H), 2.73 (t, J = 7.24 Hz, 2H), |
| | 2.03 (quin, J = 7.04 Hz, 2H); LCMS: ESI |
| | [M + H]⁺ = 334.1 |
| 220 | ¹H NMR (400 MHz, CHLOROFORM-d) δ |
| | 7.20 − 7.27 (m, 2H), 7.08 − 7.17 (m, 3H), |
| | 5.77 (br. s., 1H), 3.03 (br. s., 1H), 2.15 − |
| | 2.22 (m, 1H), 1.42 (q, J = 6.52 Hz, 1H), |
| | 1.35 (td, J = 4.99, 9.59 Hz, 1H); LCMS: |
| | ESI [M + H]⁺ = 338.0 |
| 221 | ¹H NMR (400 MHz, CHLOROFORM-d) δ |
| | 8.78 (s, 1H), 7.31 (d, J = 10.17 Hz, 1H), |
| | 7.08 − 7.18 (m, 2H), 6.52 (br. s., 1H), 3.04 |
| | (br. s., 1H), 2.18 − 2.25 (m, 1H), 1.43 (q, J = |
| | 6.78 Hz, 1H), 1.36 (td, J = 5.04, 9.88 Hz, |
| | 1H); LCMS: ESI [M + H]⁺ = 339.0 |
| 222 | ¹H NMR (400 MHz, CHLOROFORM-d) δ |
| | 7.17 − 7.26 (m, J = 9.78 Hz, 1H), 7.06 − |
| | 7.17 (m, 2H), 6.75 (s, 1H), 5.65 (br. s., 1H), |
| | 2.99 (br. s., 1H), 2.50 (s, 3H), 2.12 − 2.19 |
| | (m, 1H), 1.41 (q, J = 6.52 Hz, 1H), 1.33 (td, |
| | J = 4.99, 9.59 Hz, 1H); LCMS: ESI |
| | [M + H]⁺ = 352.0 |
| 223 | Hz, 2H), 6.61 (s, 1H), 5.10 (br. s., 1H), 3.67 |
| | ¹H NMR (400 MHz, CHLOROFORM-d) δ |
| | 7.25 (d, J = 8.22 Hz, 2H), 7.15 (d, J = 7.83 |
| | (q, J = 6.39 Hz, 2H), 2.76 (t, J = 7.43 Hz, |
| | 2H), 2.54 (s, 3H), 2.03 (quin, J = 7.14 Hz, |
| | 2H); LCMS: ESI [M + H]⁺ = 402.0 |
| 224 | ¹H NMR (400 MHz, METHANOL-d4) δ |
| | 7.29 − 7.18 (m, 4H), 7.18 − 7.10 (m, 1H), |
| | 7.05 (t, 1H), 3.55 (td, J = 7.1, 1.2 Hz, 2H), |
| | 2.71 (t, J = 7.6 Hz, 2H), 2.53 (d, J = 1.4 Hz, |
| | 3H), 2.05 − 1.97 (m, 2H); LCMS: ESI |
| | [M + H]⁺ = 318.1 |
| 225 | ¹H NMR (400 MHz, CHLOROFORM-d) |
| | 7.21 − 7.33 (m, 4H), 7.12 − 7.20 (m, 1H), |
| | 6.68 (s, 1H), 6.54 (d, J = 15.65 Hz, 1H), |
| | 6.18 − 6.29 (m, 1H), 5.29 (br. s., 1H), 4.32 |
| | (t, J = 5.87 Hz, 2H), 2.46 (s, 3H); LCMS: |
| | ESI [M + H]⁺ = 316.1 |
| 226 | ¹H NMR (400 MHz, CHLOROFORM-d) δ |
| | 7.01 − 7.16 (m, 2H), 6.99 (br. s., 1H), 6.69 |
| | (s, 1H), 5.47 (br. s., 1H), 4.69 (dq, J = 4.11, |
| | 8.41 Hz, 2H), 3.04 (d, J = 2.74 Hz, 1H), |
| | 2.48 (s, 3H), 2.08 − 2.14 (m, 1H), 1.31 − |
| | 1.45 (m, 2H); LCMS: ESI [M + H]⁺ = 416.1 |
| 227 | ¹H NMR (400 MHz, METHANOL-d4) δ |
| | 7.21 − 7.33 (m, 1H), 7.10 − 7.20 (m, 3H), |
| | 2.98 (br. s., 1H), 2.58 (s, 3H), 2.11 (t, J = |
| | 6.06 Hz, 1H), 1.29 − 1.47 (m, 2H); LCMS: |
| | ESI [M + H]⁺ = 343.1 |
| 228 | ¹H NMR (400 MHz, CHLOROFORM-d) δ |
| | 7.19 (d, J = 7.8 Hz, 1H), 7.06 − 7.00 (m, |
| | 3H), 6.58 (s, 1H), 5.10 (s, 1H), 3.70 (q, J = |
| | 6.6 Hz, 2H), 2.73 (t, J = 7.5 Hz, 2H), 2.58 |
| | (s, 3H), 2.32 (s, 3H), 2.04 (quin, J = 7.3 Hz, |
| | 2H); LCMS: ESI [M + H]⁺ = 366.0 |
| 229 | ¹H NMR (400 MHz, CHLOROFORM-d) δ |
| | 7.90 − 7.84 (m, 2H), 7.39 (dd, J = 18.7, 7.6 |
| | Hz, 2H), 6.69 (s, 1H), 5.17 (s, 1H), 3.91 (t, |
| | J = 1.8 Hz, 3H), 3.71 (q, J = 6.7 Hz, 2H), |
| | 2.80 (t, J = 7.6 Hz, 2H), 2.58 (s, 3H), 2.07 |
| | (quin, J = 7.3 Hz, 2H); LCMS: ESI |
| | [M + H]⁺ = 410.0 |
| 230 | ¹H NMR (400 MHz, CHLOROFORM-d) δ |
| | 7.76 (d, J = 7.8 Hz, 1H), 7.66 (d, J = 7.7 |
| | Hz, 1H), 7.31 (q, J = 8.8, 8.0 Hz, 2H), 7.07 |
| | (s, 1H), 6.32 (s, 1H), 5.13 (s, 1H), 3.79 (q, |
| | J = 6.1 Hz, 2H), 3.06 (d, J = 7.2 Hz, 2H), |
| | 2.46 (s, 3H), 2.19 (q, J = 6.8 Hz, 2H); |
| | LCMS: ESI [M + H]⁺ = 408.0 |
| 231 | ¹H NMR (400 MHz, CHLOROFORM-d) δ |
| | 7.25 (d, J = 7.5 Hz, 1H), 6.99 (d, J = 7.7 |
| | Hz, 1H), 6.95 − 6.85 (m, 2H), 6.73 − 6.68 |
| | (m, 1H), 5.19 (d, J = 6.8 Hz, 1H), 3.70 (q, |
| | J = 6.6 Hz, 2H), 2.75 (t, J = 7.6 Hz, 2H), 2.59 |
| | (d, J = 2.3 Hz, 3H), 2.04 (quin, J = 7.2 Hz, |
| | 2H); LCMS: ESI [M + H]⁺ = 370.1 |
| 232 | ¹H NMR (400 MHz, CHLOROFORM-d) δ |
| | 7.79 (dt, J = 15.6, 4.7 Hz, 3H), 7.66 (s, 1H), |
| | 7.47 (dd, J = 7.4, 3.7 Hz, 2H), 7.40 − 7.33 |
| | (m, 1H), 6.18 (s, 1H), 5.00 (s, 1H), 3.75 (t, |
| | J = 6.4 Hz, 2H), 2.95 (t, J = 7.2 Hz, 2H), |
| | 2.43 (s, 3H), 2.15 (quin, J = 7.0 Hz, 2H); |
| | LCMS: ESI [M + H]⁺ = 402.1 |
| 233 | ¹H NMR (400 MHz, CHLOROFORM-d) δ |
| | 7.14 (d, J = 8.0 Hz, 2H), 7.03 − 6.95 (m, |
| | 2H), 6.59 (s, 1H), 5.05 (s, 1H), 3.74 − 3.65 |
| | (m, 3H), 2.72 (t, J = 7.4 Hz, 2H), 2.58 (s, |
| | 3H), 2.02 (quin, J = 7.0 Hz, 2H), 0.76 (d, J = |
| | 5.6 Hz, 4H); LCMS: ESI [M + H]⁺ = 408.1 |
| 234 | ¹H NMR (400 MHz, CHLOROFORM-d) δ |
| | 7.15 (s, 4H), 6.58 (s, 1H), 5.08 (s, 1H), 3.70 |
| | (q, J = 6.3 Hz, 2H), 2.74 (t, J = 7.4 Hz, 2H), |
| | 2.57 (s, 3H), 2.48 (s, 1H), 2.04 (quin, J = |
| | 7.1 Hz, 2H), 1.84 (d, J = 10.6 Hz, 4H), 1.77 |
| | (s, 1H), 1.42 (q, J = 11.8, 10.7 Hz, 4H), |
| | 1.25 (d, J = 11.1 Hz, 1H); LCMS: ESI |
| | [M + H]⁺ = 434.1 |
| 235 | ¹H NMR (400 MHz, CHLOROFORM-d) δ |
| | 7.02 (d, J = 7.8 Hz, 2H), 6.65 (d, J = 7.7 |
| | Hz, 2H), 6.57 (s, 1H), 5.05 (s, 1H), 3.69 (q, |
| | J = 6.2 Hz, 4H), 2.66 (d, J = 7.0 Hz, 2H), |
| | 2.58 (s, 3H), 1.99 (t, J = 7.0 Hz, 2H); |
| | LCMS: ESI [M + H]⁺ = 367.1 |
| 236 | ¹H NMR (400 MHz, CHLOROFORM-d) δ |
| | 7.18 − 7.08 (m, 4H), 6.58 (s, 1H), 5.09 (s, |
| | 1H), 3.70 (q, J = 6.1 Hz, 2H), 2.73 (d, J = |
| | 7.5 Hz, 2H), 2.57 (s, 5H), 2.04 (quin, J = |
| | 7.2 Hz, 2H), 1.64 (d, J = 7.7 Hz, 2H), 0.99 − |
| | 0.91 (m, 3H); LCMS: ESI [M + H]⁺ = 394.1 |
| 237 | ¹H NMR (400 MHz, CHLOROFORM-d) δ |
| | 7.12 (d, J = 7.7 Hz, 2H), 7.01 (d, J = 7.7 |
| | Hz, 2H), 6.52 (s, 1H), 5.01 (s, 1H), 3.70 (d, |
| | J = 6.3 Hz, 2H), 2.73 (t, J = 7.4 Hz, 2H), |
| | 2.58 (s, 3H), 2.03 (t, J = 7.1 Hz, 2H), 1.88 |
| | (s, 1H), 0.99 − 0.92 (m, 2H), 0.67 (d, J = 5.2 |
| | Hz, 2H); LCMS: ESI [M + H]⁺ = 392.0 |
| 238 | ¹H NMR (400 MHz, CHLOROFORM-d) δ |
| | 7.56 (d, J = 7.6 Hz, 2H), 7.32 (d, J = 7.7 |
| | Hz, 2H), 6.73 (s, 1H), 5.26 (d, J = 7.0 Hz, |
| | 1H), 3.70 (q, J = 6.6 Hz, 2H), 2.80 (t, J = |
| | 7.6 Hz, 2H), 2.59 (s, 3H), 2.05 (h, J = 6.8, |
| | 6.2 Hz, 2H); LCMS: ESI [M + H]⁺ = 377.3 |
| 239 | ¹H NMR (400 MHz, CHLOROFORM-d) δ |
| | 7.19 − 7.12 (m, 1H), 6.95 (dd, J = 5.1, 3.2 |
| | Hz, 1H), 6.86 (s, 1H), 6.70 (d, J = 2.4 Hz, |
| | 1H), 5.18 (s, 1H), 3.74 (q, J = 6.6 Hz, 2H), |
| | 2.98 (t, J = 7.4 Hz, 2H), 2.62 − 2.57 (m, |
| | 3H), 2.10 (quin, J = 7.2 Hz, 2H); LCMS: |
| | ESI [M + H]⁺ = 358.3 |
| 240 | ¹H NMR (400 MHz, CHLOROFORM-d) δ |
| | 7.29 − 7.23 (s 1H), 7.13 (dd, J = 17.4, 8.9 |
| | Hz, 2H), 6.77 (d, J = 2.5 Hz, 1H), 5.23 (s, |
| | 1H), 3.73 (q, J = 6.6 Hz, 2H), 2.85 − 2.77 |
| | (m, 2H), 2.60 (s, 3H), 2.08 (q, J = 7.4 Hz, |
| | 2H); LCMS: ESI [M + H]⁺ = 438.2 |
| 241 | ¹H NMR (400 MHz, CHLOROFORM-d) δ |
| | 7.22 (t, J = 8.2 Hz, 1H), 6.82 (d, J = 7.4 Hz, |
| | 1H), 6.76 (d, J = 7.5 Hz, 2H), 6.62 (s, 1H), |
| | 5.14 (s, 1H), 3.78 (s, 3H), 3.70 (q, J = 6.5 |
| | Hz, 2H), 2.74 (t, J = 7.4 Hz, 2H), 2.58 (s, |
| | 3H), 2.05 (quin, J = 7.2 Hz, 2H); LCMS: |
| | ESI [M + H]⁺ = 382.0 |
| 242 | ¹H NMR (400 MHz, CHLOROFORM-d) δ |
| | 7.90 − 7.82 (m, 2H), 7.42 (d, J = 7.8 Hz, |
| | 2H), 6.75 (s, 1H), 5.24 (s, 1H), 3.71 (q, J = |
| | 6.8 Hz, 2H), 3.04 (d, J = 2.1 Hz, 3H), 2.84 |
| | (t, J = 7.6 Hz, 2H), 2.60 (s, 3H), 2.08 (quin, |
| | J = 7.7 Hz, 2H); LCMS: ESI [M + H]⁺ = |
| | 430.1 |
| 243 | ¹H NMR (400 MHz, CHLOROFORM-d) δ |
| | 7.52 − 7.43 (m, 3H), 7.38 (t, J = 7.9 Hz, |
| | 1H), 6.78 (s, 1H), 5.24 (s, 1H), 3.71 (q, J = |
| | 6.8 Hz, 2H), 2.78 (t, J = 7.7 Hz, 2H), 2.61 |
| | (s, 3H), 2.04 (q, J = 7.6 Hz, 2H); LCMS: |
| | ESI [M + H]⁺ = 377.1 |
| 244 | ¹H NMR (400 MHz, CHLOROFORM-d) |
| | 7.12 (d, J = 2.4 Hz, 4H), 6.55 (s, 1H), 5.06 |
| | (s, 1H), 3.70 (q, J = 6.2 Hz, 2H), 2.77 − 2.69 |
| | (m, 2H), 2.57 (s, 3H), 2.34 (s, 3H), 2.03 |
| | (quin, J = 6.8 Hz, 2H); LCMS: ESI [M + |
| | H]⁺ = 366.0 |
| 245 | ¹H NMR (400 MHz, CHLOROFORM-d) δ |
| | 7.25 (d, J = 7.6 Hz, 2H), 7.15 (d, J = 8.0 |
| | Hz, 2H), 6.62 (s, 1H), 5.10 (s, 1H), 3.69 (q, |
| | J = 6.7 Hz, 2H), 2.73 (t, J = 7.5 Hz, 2H), |
| | 2.58 (s, 3H), 2.02 (quin, J = 7.4 Hz, 2H); |
| | LCMS: ESI [M + H]⁺ = 386.1 |
| 246 | ¹H NMR (400 MHz, CHLOROFORM-d) δ |
| | 7.96 (d, J = 7.7 Hz, 2H), 7.31 − 7.24 (m, |
| | 2H), 6.64 (s, 1H), 5.13 (s, 1H), 3.90 (d, J = |
| | 2.5 Hz, 3H), 3.71 (q, J = 6.5 Hz, 2H), 2.81 |
| | (t, J = 7.6 Hz, 2H), 2.57 (s, 3H), 2.07 (quin, |
| | J = 7.4 Hz, 2H); LCMS: ESI [M + H]⁺ = |
| | 410.1 |
| 247 | ¹H NMR (400 MHz, CHLOROFORM-d) δ |
| | 7.20 − 7.12 (m, 4H), 6.64 (s, 1H), 5.14 (s, |
| | 1H), 3.73 (q, J = 6.6 Hz, 2H), 2.76 (t, J = |
| | 7.7 Hz, 2H), 2.58 (s, 3H), 2.31 (d, J = 2.8 |
| | Hz, 3H), 2.01 (quin, J = 7.5 Hz, 2H); |
| | LCMS: ESI [M + H]⁺ = 366.1 |
| 248 | ¹H NMR (400 MHz, CHLOROFORM-d) δ |
| | 7.19 (d, J = 4.8 Hz, 3H), 7.09 (d, J = 7.2 |
| | Hz, 1H), 6.70 (s, 1H), 5.17 (s, 1H), 3.70 (q, |
| | J = 6.3 Hz, 2H), 2.77 − 2.69 (m, 2H), 2.59 |
| | (s, 3H), 2.04 (quin, J = 7.9, 7.4 Hz, 2H); |
| | LCMS: ESI [M + H]⁺ = 386.1 |
| 249 | ¹H NMR (400 MHz, CHLOROFORM-d) δ |
| | 7.35 (d, J = 7.7 Hz, 1H), 7.29 − 7.26 (m, |
| | 1H), 7.23 − 7.11 (m, 2H), 6.75 (s, 1H), 5.28 |
| | (s, 1H), 3.71 (q, J = 6.3 Hz, 2H), 2.87 (t, J = |
| | 7.6 Hz, 2H), 2.60 (s, 3H), 2.06 (q, J = 7.3 |
| | Hz, 2H); LCMS: ESI [M + H]⁺ = 386.1 |
| 250 | ¹H NMR (400 MHz, CHLOROFORM-d) δ |
| | 7.11 (d, J = 8.0 Hz, 2H), 6.71 (d, J = 7.9 |
| | Hz, 2H), 6.46 (s, 1H), 4.88 (s, 1H), 3.66 (q, |
| | J = 6.4 Hz, 2H), 2.93 (d, J = 2.5 Hz, 6H), |
| | 2.82 (q, J = 7.8 Hz, 2H), 2.67 (d, J = 7.7 |
| | Hz, 2H), 2.50 (s, 3H), 1.98 (t, J = 7.1 Hz, |
| | 2H), 1.34 (t, J = 7.7 Hz, 3H); LCMS: ESI |
| | [M + H]⁺ =355.2 |
| 251 | ¹H NMR (400 MHz, CHLOROFORM-d) δ |
| | 7.14 − 7.09 (m, 4H), 6.52 (q, J = 1.3 Hz, |
| | 1H), 4.81 (s, 1H), 3.71 − 3.61 (m, 2H), 2.82 |
| | (qt, J = 7.6, 1.1 Hz, 2H), 2.72 (t, J = 7.4 Hz, |
| | 2H), 2.52 (q, J = 1.2 Hz, 3H), 2.34 (d, J = |
| | 1.5 Hz, 3H), 2.01 (quin, J = 7.2 Hz, 2H), |
| | 1.34 (tt, J = 7.6, 1.1 Hz, 3H); LCMS: ESI |
| | [M + H]⁺ = 326.2 |
| 252 | ¹H NMR (400 MHz, CHLOROFORM-d) δ |
| | 7.21 (t, J = 8.3 Hz, 1H), 7.07 (dt, J = 11.0, |
| | 2.1 Hz, 1H), 6.99 (d, J = 8.2 Hz, 1H), 6.68 − |
| | 6.63 (m, 1H), 4.89 (s, 1H), 3.72 − 3.62 (m, |
| | 2H), 2.83 (qd, J = 7.6, 2.0 Hz, 2H), 2.78 − |
| | 2.70 (m, 2H), 2.54 (dd, J = 2.2, 1.2 Hz, |
| | 3H), 2.08 − 1.95 (m, 2H), 1.34 (td, J = 7.6, |
| | 2.1 Hz, 3H); LCMS: ESI [M + H]⁺ = 414.2 |
| 253 | ¹H NMR (400 MHz, CHLOROFORM-d) δ |
| | 7.18 (td, J = 7.8, 1.8 Hz, 1H), 6.65 − 6.54 |
| | (m, 3H), 6.49 (q, J = 1.4 Hz, 1H), 4.84 (s, |
| | 1H), 3.73 − 3.63 (m, 2H), 2.94 − 2.89 (m, |
| | 6H), 2.82 (tdd, J = 7.7, 7.0, 1.7 Hz, 2H), |
| | 2.77 − 2.68 (m, 2H), 2.51 (q, J = 1.3 Hz, |
| | 3H), 2.04 (pd, J = 7.1, 1.9 Hz, 2H), 1.38 − |
| | 1.29 (m, 3H); LCMS: ESI [M + H]⁺ = 355.2 |
| 254 | ¹H NMR (400 MHz, CHLOROFORM-d) δ |
| | 8.88 (dt, J = 4.2, 1.7 Hz, 1H), 8.10 − 8.01 |
| | (m, 2H), 7.61 (dp, J = 3.9, 1.9 Hz, 2H), |
| | 7.38 (ddd, J = 8.3, 4.3, 1.6 Hz, 1H), 6.46 (d, |
| | J = 1.7 Hz, 1H), 4.85 (s, 1H), 3.72 (q, J = |
| | 6.6 Hz, 2H), 2.95 (t, J = 7.5 Hz, 2H), 2.82 |
| | (qd, J = 7.6, 1.6 Hz, 2H), 2.47 (d, J = 1.4 |
| | Hz, 3H), 2.14 (quin, J = 7.2 Hz, 2H), 1.33 |
| | (td, J = 7.6, 1.6 Hz, 3H); LCMS: ESI |
| | [M + H]⁺ = 363.2 |
| 255 | ¹H NMR (400 MHz, CHLOROFORM-d) δ |
| | 7.30 (t, J = 7.8 Hz, 1H), 7.17 − 7.11 (m, |
| | 1H), 7.06 (d, J = 9.2 Hz, 2H), 6.64 (t, J = |
| | 1.4 Hz, 1H), 5.21 (s, 1H), 3.67 (q, J = 6.7 |
| | Hz, 2H), 2.88 − 2.72 (m, 4H), 2.52 (t, J = |
| | 1.3 Hz, 3H), 2.02 (q, J = 7.3 Hz, 2H), 1.34 |
| | (td, J = 7.6, 1.3 Hz, 3H); LCMS: ESI |
| | [M + H]⁺ = 396.1 |
| 256 | ¹H NMR (400 MHz, CHLOROFORM-d) δ |
| | 7.04 (s, 1H), 6.96 (d, J = 8.1 Hz, 1H), 6.73 |
| | (d, J = 8.1 Hz, 1H), 6.54 − 6.49 (m, 1H), |
| | 4.82 (s, 1H), 4.54 (td, J = 8.6, 1.4 Hz, 2H), |
| | 3.66 (dt, J = 8.3, 6.1 Hz, 2H), 3.14 (t, J = |
| | 8.7 Hz, 2H), 2.82 (qd, J = 7.6, 1.4 Hz, 2H), |
| | 2.69 (t, J = 7.4 Hz, 2H), 2.52 (t, J = 1.3 Hz, |
| | 3H), 1.98 (quin, J = 7.1 Hz, 2H), 1.34 (td, |
| | J = 7.6, 1.4 Hz, 3H); LCMS: ESI [M + |
| | H]⁺ = 354.2 |
| 257 | ¹H NMR (400 MHz, CHLOROFORM-d) δ |
| | 7.29 − 7.22 (m, 2H), 7.14 (dd, J = 8.4, 1.8 |
| | Hz, 2H), 6.59 − 6.53 (m, 1H), 4.85 (s, 1H), |
| | 3.65 (dt, J = 8.7, 6.1 Hz, 2H), 2.82 (qd, J = |
| | 7.6, 1.7 Hz, 2H), 2.71 (t, J = 7.8 Hz, 2H), |
| | 2.55 − 2.50 (m, 3H), 2.06 − 1.94 (m, 2H), |
| | 1.33 (td, J = 7.6, 1.7 Hz, 3H); LCMS: ESI |
| | [M + H]⁺ = 346.2 |
| 258 | ¹H NMR (400 MHz, CHLOROFORM-d) δ |
| | 7.81 − 7.70 (m, 3H), 7.63 (s, 1H), 7.42 (pd, |
| | J = 6.8, 4.9 Hz, 2H), 7.37 − 7.29 (m, 1H), |
| | 6.25 (s, 1H), 4.85 − 4.78 (m, 1H), 3.68 (dt, |
| | J = 8.6, 6.0 Hz, 2H), 2.94 − 2.86 (m, 2H), |
| | 2.78 (qd, J = 7.8, 2.0 Hz, 2H), 2.38 (q, J = |
| | 1.2 Hz, 3H), 2.15 − 2.04 (m, 2H), 1.30 (td, |
| | J = 7.6, 1.9 Hz, 3H); LCMS: ESI [M + H]⁺ = |
| | 362.2 |
| 259 | ¹H NMR (400 MHz, CHLOROFORM-d) δ |
| | 7.26 (d, J = 2.8 Hz, 1H), 7.14 (t, J = 10.6 |
| | Hz, 2H), 6.67 (q, J = 1.6 Hz, 1H), 4.96 (s, |
| | 1H), 3.73 − 3.63 (m, 2H), 2.89 − 2.76 (m, |
| | 4H), 2.53 (t, J = 1.4 Hz, 3H), 2.04 (quin, |
| | J = 7.7 Hz, 2H), 1.38 − 1.29 (m, 3H); LCMS: |
| | ESI [M + H]⁺ = 398.1 |
| 260 | ¹H NMR (400 MHz, CHLOROFORM-d) δ |
| | 6.96 (d, J = 12.2 Hz, 1H), 6.93 − 6.82 (m, |
| | 2H), 6.61 (dq, J = 2.6, 1.3 Hz, 1H), 4.87 (s, |
| | 1H), 3.87 (d, J = 2.3 Hz, 3H), 3.65 (q, J = |
| | 6.3 Hz, 2H), 2.83 (qd, J = 7.6, 2.1 Hz, 2H), |
| | 2.68 (td, J = 7.3, 1.9 Hz, 2H), 2.53 (q, J = |
| | 1.3 Hz, 3H), 2.04 − 1.92 (m, 2H), 1.34 (td, |
| | J = 7.6, 2.1 Hz, 3H); LCMS: ESI [M + H]⁺ = |
| | 360.2 |
| 261 | ¹H NMR (400 MHz, CHLOROFORM-d) δ |
| | 7.77 (d, J = 7.8 Hz, 1H), 7.66 (d, J = 7.8 |
| | Hz, 1H), 7.31 (ddd, J = 12.2, 8.1, 6.6 Hz, |
| | 2H), 7.06 (s, 1H), 6.39 (d, J = 1.7 Hz, 1H), |
| | 4.92 (s, 1H), 3.74 (q, J = 6.4 Hz, 2H), 3.05 |
| | (t, J = 7.2 Hz, 2H), 2.83 (q, J = 7.6 Hz, 2H), |
| | 2.44 (d, J = 1.2 Hz, 3H), 2.15 (quin, J = 7.0 |
| | Hz, 2H), 1.34 (td, J = 7.6, 1.1 Hz, 3H); |
| | LCMS: ESI [M + H]⁺ = 368.2 |

### Example 4: Scheme 2 S_{N}Ar Reaction+Suzuki Reaction

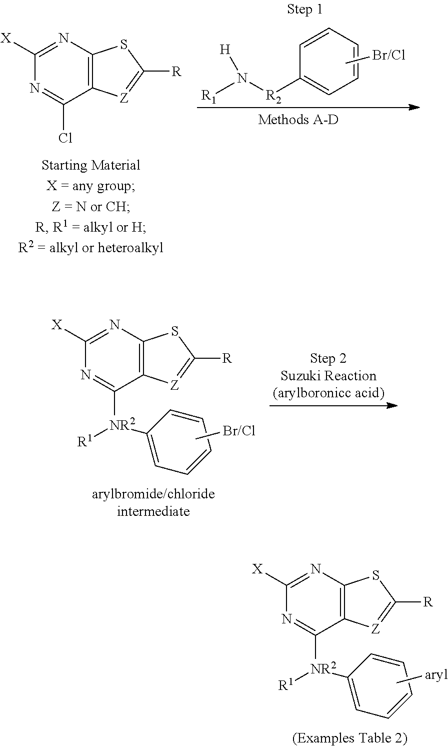

General Procedure for Scheme 2: Reaction conditions and compound data for specific examples are listed in Table 2. Starting materials were synthesized as detailed in the building blocks section, unless otherwise stated.

Step 1: The specified starting material (0.056 mmol) was reacted with the appropriate commercially available bromo (or chloro)phenylamine derivative following one of Scheme 1, Methods A-D (details in Table 2). Upon completion, the reaction was concentrated in vacuo, and purified by silica gel column chromatography to give the intermediate aryl halide derivative for use in step 2.

Step 2: Following the General Suzuki Protocol, the aryl halide intermediate (0.030 mmol) and the desired commercially available aryl boronic acid (0.060 mmol) were reacted for the specified time and temperature. The reaction was purified by prep HPLC or silica gel column chromatography, or a combination of the two, to give the pure product (Table 2).

TABLE 2
Examples from Scheme 2

| | | | Step 1 | | | | | |
|---|---|---|---|---|---|---|---|---|
| Cmpd. | | Starting | Method/ | Step 1 | Step 2 | Step 2 | | |
| No. | Structure | Material | Conditions | Yield | Conditions | Yield | IUPAC | Compound Data |
| 262 | 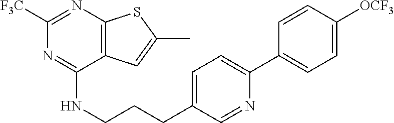 [SMILES: Cc1cc2c(NCCCc3ccc(-c4ccc(OC(F)(F)F)cc4)nc3)nc(C(F)(F)F)nc2s1] | KH 6080 | Method C 5 h (25° C.) | 55% | Cs₂CO₃ 2 h (80° C.) | 24% | 6-methyl-N-(3- (6-(4- (trifluoromethoxy) phenyl)pyridin-3- yl)propyl)-2- (trifluoromethyl) thieno[2,3- d]pyrimidin-4- | ¹H NMR (400 MHz, CHLOROFORM-d) δ 8.57 (s, 1H), 8.00 (d, J = 8.6 Hz, 2H), 7.69-7.59 (m, 2H), 7.31 (d, J = 8.4 Hz, 2H), 6.72 (t, J = 1.2 Hz, 1H), 5.25 (s, 1H), 3.75 (q, J = 6.6 Hz, 2H), 2.82 (t, J = 7.4 Hz, 2H), 2.52 (d, J = 1.2 Hz, 3H), 2.11 |
| | | | | | | | amine; | (quin, J = 7.1 Hz, 2H); LCMS: |
| | | | | | | | | ESI [M + H]⁺ = 513.1 |
| 263 | 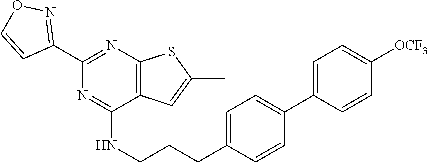 [SMILES: Cc1cc2c(NCCCc3ccc(-c4ccc(OC(F)(F)F)cc4)cc3)nc(-c3ccon3)nc2s1] | KH 7018 | Method A 1 d (60° C.) | 90% | Cs₂CO₃ 2 h (80° C.) | 43% | 2-(isoxazol-3- yl)-6-methyl- N-(3-(4′- (trifluorometho xy)-[1,1′- biphenyl]-4- yl)propyl)thieno [2,3- d]pyrimidin-4- amine | ¹H NMR (400 MHz, CHLOROFORM-d) δ 8.45 (s, 1H), 7.56-7.66 (m, 2H), 7.46- 7.56 (m, 2H), 7.27-7.35 (m, 4H), 6.98 (s, 1H), 6.61 (s, 1H), 5.10 (br. s., 1H), 3.70-3.91 (m, 2H), 2.84 (t, J = 7.04 Hz, 2H), 2.48 (s, 3H), 2.12 (quin, J = 6.75 Hz, 2H); LCMS: ESI [M + H^{]+} = 511.2 |
| 264 | 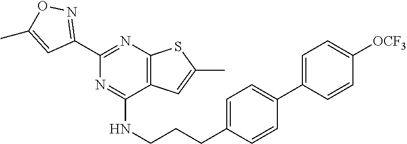 [SMILES: Cc1cc(-c2nc(NCCCc3ccc(-c4ccc(OC(F)(F)F)cc4)cc3)c3cc(C)sc3n2)no1] | KH 7019 | Method A 1 d (60° C.) | 87% | Cs₂CO₃ 2 h (80° C.) | 47% | 6-methyl-2-(5- methylisoxazol- 3-yl)-N-(3-(4′- (trifluoromethoxy)- [1,1′-biphenyl]-4- yl)propyl)thieno [2,3- d]pyrimidin-4- amine | ¹H NMR (400 MHz, CHLOROFORM-d) δ 7.59 (dd, J = 1.96, 8.61 Hz, 2H), 7.47-7.55 (m, 2H), 7.27-7.34 (m, 4H), 6.62 (br. s., 2H), 4.92-5.35 (m, 1H), 3.78 (q, J = 5.61 Hz, 2H), 2.83 (t, J = 7.04 Hz, 2H), 2.47 (s, 6H), 2.05-2.18 (m, 2H); LCMS: ESI [M + H]⁺ = 525.2 |
| 265 | 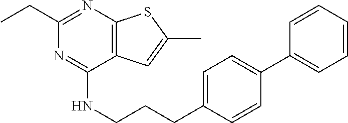 [SMILES: CCc1nc(NCCCc2ccc(-c3ccccc3)cc2)c2cc(C)sc2n1] | KH 4007 | Method A 2 d (80° C.) | 86% | Cs₂CO₃ 2 h (80° C.) | 56% | N-(3-([1,1′- biphenyl]-4- yl)propyl)-2- ethyl-6- methylthieno[2, 3-d]pyrimidin- 4-amine | ¹H NMR (400 MHz, CHLOROFORM-d) δ 7.63-7.51 (m, 4H), 7.44 (td, J = 7.7, 2.0 Hz, 2H), 7.39-7.28 (m, 3H), 6.52- 6.47 (m, 1H), 4.82 (s, 1H), 3.71 (qd, J = 6.7, 2.1 Hz, 2H), 2.89- 2.77 (m, 4H), 2.43 (q, J = 1.2 Hz, 3H), 2.07 (pd, J = 7.0, 2.1 Hz, |
| | | | | | | | | 2H), 1.35 (td, J = 7.6, 2.1 Hz, |
| | | | | | | | | 3H); LCMS: ESI [M + H]⁺ = 388.2 |
| 266 | 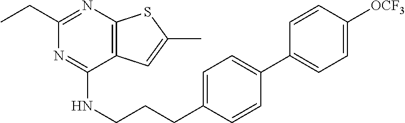 [SMILES: CCc1nc(NCCCc2ccc(-c3ccc(OC(F)(F)F)cc3)cc2)c2cc(C)sc2n1] | KH 4007 | Method A 2 d (80° C.) | 86% | Cs₂CO₃ 2 h (80° C.) | 38% | 2-ethyl-6- methyl-N-(3-(4′- (trifluoromethoxy)- [1,1′-biphenyl]-4- yl)propyl)thieno [2,3- d]pyrimidin-4- amine | ¹H NMR (400 MHz, CHLOROFORM-d) δ 7.62-7.54 (m, 2H), 7.53-7.46 (m, 2H), 7.29 (ddd, J = 11.6, 8.6, 2.3 Hz, 4H), 6.55-6.50 (m, 1H), 4.81 (s, 1H), 3.70 (q, J = 6.8 Hz, 2H), 2.89- 2.77 (m, 4H), 2.47-2.42 (m, 3H), 2.07 (quin, J = 7.1 Hz, 2H), 1.39- |
| | | | | | | | | 1.30 (m, 3H); LCMS: ESI |
| | | | | | | | | [M + H]⁺ = 472.2 |
| 267 | 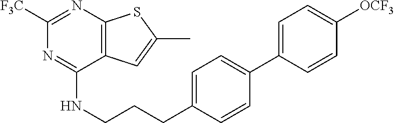 [SMILES: Cc1cc2c(NCCCc3ccc(-c4ccc(OC(F)(F)F)cc4)cc3)nc(C(F)(F)F)nc2s1] | KH 6080 | Method A 1 d (60° C.) | 93% | Cs₂CO₃ 3 h (80° C.) | 86% | 6-methyl-N-(3-(4′- (trifluoromethoxy)- [1,1′-biphenyl]-4- yl)propyl)-2- (trifluoromethyl)- thieno[2,3- d]pyrimidin-4- amine | ¹H NMR (400 MHz, CHLOROFORM-d) δ 7.61-7.53 (m, 2H), 7.49 (dt, J = 8.2, 1.8 Hz, 2H), 7.33-7.22 (m, 4H), 6.56 (d, J = 2.4 Hz, 1H), 5.10 (s, 1H), 3.72 (qd, J = 6.7, 6.0, 1.9 Hz, 2H), 2.80 (t, J = 7.4 Hz, 2H), 2.46 (d, J = 2.5 Hz, 3H), 2.07 (tt, J = |
| | | | | | | | | 8.2, 4.9 Hz, 2H); LCMS: ESI |
| | | | | | | | | [M + H]⁺ = 512.2 |
| 268 | 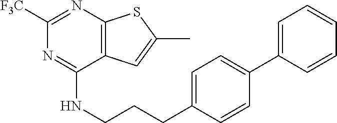 | KH 6080 | Method A 1 d (60° C.) | 93% | Cs₂CO₃ 3 h (80° C.) | 57% | N-(3-([1,1′- biphenyl]-4- yl)propyl)-6- methyl-2- (trifluoromethyl) thieno[2,3- d]pyrimidin-4- amine | ¹H NMR (400 MHz, CHLOROFORM-d) δ 7.57 (ddd, J = 15.2, 7.8, 1.6 Hz, 4H), 7.49- 7.40 (m, 2H), 7.37-7.28 (m, 3H), 6.50 (s, 1H), 5.05 (s, 1H), 3.74 (q, J = 6.3 Hz, 2H), 2.82 (t, J = 7.3 Hz, 2H), 2.44 (s, 3H), 2.10 (dd, J = 7.9, 6.1 Hz, 2H); LCMS: ESI |
| | | | | | | | | [M + H]⁺ = 428.1 |
| 269 | 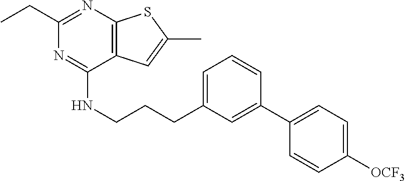 [SMILES: CCc1nc(NCCCc2cccc(-c3ccc(OC(F)(F)F)cc3)c2)c2cc(C)sc2n1] | KH 4007 | Method A 2 d (80° C.) | 86% | Cs₂CO₃ 2 h (80° C.) | 44% | 2-ethyl-6- methyl-N-(3- (4′- (trifluoromethoxy)- [1,1′- biphenyl]-3- yl)propyl)thieno [2,3- d]pyrimidin-4- amine | ¹H NMR (400 MHz, CHLOROFORM-d) δ 7.60-7.51 (m, 2H), 7.43-7.33 (m, J = 1.6 Hz, 3H), 7.30-7.19 (m, 3H), 6.58 (d, J = 1.4 Hz, 1H), 5.01 (s, 1H), 3.70 (q, J = 6.6, 6.1 Hz, 2H), 2.86-2.76 (m, 4H), 2.49 (s, 3H), 2.07 (quin, J = 7.3 Hz, 2H), 1.32 (td, J = 7.6, 1.2 Hz, 3H); LCMS: ESI [M + H]⁺ = 472.2 |
| 270 | 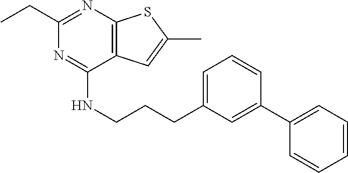 [SMILES: CCc1nc(NCCCc2cccc(-c3ccccc3)c2)c2cc(C)sc2n1] | KH 4007 | Method A 2 d (80° C.) | 86% | Cs₂CO₃ 2 h (80° C.) | 52% | N-(3-([1,1′- biphenyl]-3- yl)propyl)-2- ethyl-6- methylthieno[2, 3-d]pyrimidin- 4-amine | ¹H NMR (400 MHz, CHLOROFORM-d) δ 7.60-7.52 (m, 2H), 7.48-7.39 (m, 4H), 7.39- 7.30 (m, 2H), 7.21 (dd, J = 7.4, 1.7 Hz, 1H), 6.56 (dd, J = 2.7, 1.4 Hz, 1H), 4.95 (s, 1H), 3.70 (dt, J = 9.0, 6.1 Hz, 2H), 2.82 (qd, J = 7.5, 1.9 Hz, 4H), 2.49 (t, J = 1.6 Hz, 3H), 2.08 (qd, J = 7.7, 3.8 Hz, 2H), 1.33 (td, J = 7.6, 2.0 Hz, 3H); LCMS: ESI [M + H]⁺ = 388.2 |
| 271 | 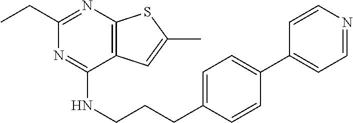 [SMILES: CCc1nc(NCCCc2ccc(-c3ccncc3)cc2)c2cc(C)sc2n1] | KH 4007 | Method A 2 d (80° C.) | 86% | Cs₂CO₃ 3 h (80° C.) | 52% | 2-ethyl-6- methyl-N-(3- (4-(pyridin-4- yl)phenyl)propyl) thieno[2,3- d]pyrimidin-4- amine | ¹H NMR (400 MHz, CHLOROFORM-d) δ 8.65 (s, 2H), 7.58 (dd, J = 8.2, 2.1 Hz, 2H), 7.49 (dd, J = 4.4, 2.0 Hz, 2H), 7.34 (dd, J = 8.2, 2.1 Hz, 2H), 6.55 (d, J = 2.1 Hz, 1H), 4.89 (s, 1H), 3.75-3.65 (m, 2H), 2.88-2.77 (m, 4H), 2.46 (t, J = |
| | | | | | | | | 1.7 Hz, 3H), 2.08 (q, J = 7.3 Hz, |
| | | | | | | | | 2H), 1.34 (td, J = 7.6, 2.0 Hz, |
| | | | | | | | | 3H); LCMS: ESI [M + H]⁺ = 389.2 |
| 272 | 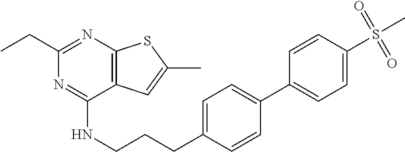 [SMILES: CCc1nc(NCCCc2ccc(-c3ccc(S(C)(=O)=O)cc3)cc2)c2cc(C)sc2n1] | KH 4007 | Method A 2 d (80° C.) | 86% | Cs₂CO₃ 3 h (80° C.) | 42% | 2-ethyl-6- methyl-N-(3-(4′- (methylsulfonyl)- [1,1′- biphenyl]-4- yl)propyl)thieno [2,3- d]pyrimidin-4- amine | ¹H NMR (400 MHz, CHLOROFORM-d) δ 8.00 (dd, J = 8.5, 2.0 Hz, 2H), 7.79-7.71 (m, 2H), 7.55 (dd, J = 8.2, 2.0 Hz, 2H), 7.34 (dd, J = 8.3, 2.0 Hz, 2H), 6.58 (dd, J = 2.7, 1.4 Hz, 1H), 4.90 (s, 1H), 3.70 (q, J = 6.9 Hz, 2H), 3.10 (d, J = 2.0 Hz, 3H), 2.83 (dtt, J = 7.5, 3.5, 1.8 Hz, |
| | | | | | | | | 4H), 2.47 (t, J = 1.3 Hz, 3H), 2.13- |
| | | | | | | | | 1.98 (m, 2H), 1.34 (td, J = 7.6, |
| | | | | | | | | 2.0 Hz, 3H); LCMS: ESI |
| | | | | | | | | [M + H]⁺ = 466.2 |
| 273 | 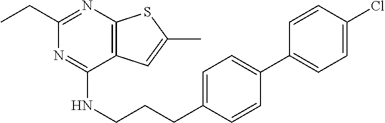 [SMILES: CCc1nc(NCCCc2ccc(-c3ccc(Cl)cc3)cc2)c2cc(C)sc2n1] | KH 4007 | Method A 2 d (80° C.) | 86% | Cs₂CO₃ 3 h (80° C.) | 30% | N-(3-(4′- chloro-[1,1′- biphenyl]-4- yl)propyl)-2- ethyl-6- methylthieno[2, 3-d]pyrimidin- 4-amine | ¹H NMR (400 MHz, CHLOROFORM-d) δ 7.54-7.46 (m, 4H), 7.43-7.36 (m, 2H), 7.29 (d, J = 7.8 Hz, 2H), 6.55-6.49 (m, 1H), 4.85 (s, 1H), 3.69 (q, J = 6.3 Hz, 2H), 2.88-2.76 (m, 4H), 2.45 (d, J = 1.3 Hz, 3H), 2.12- 1.98 (m, 2H), 1.34 (td, J = 7.5, |
| | | | | | | | | 1.0 Hz, 3H); LCMS: ESI |
| | | | | | | | | [M + H]⁺ = 422.2 |
| 274 | 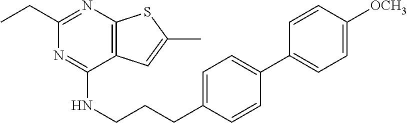 [SMILES: CCc1nc(NCCCc2ccc(-c3ccc(OC)cc3)cc2)c2cc(C)sc2n1] | KH 4007 | Method A 2 d (80° C.) | 86% | Cs₂CO₃ 2 h (80° C.) | 52% | 2-ethyl-N-(3- (4′-methoxy- [1,1′-biphenyl]- 4-yl)propyl)-6- methylthieno[2, 3-d]pyrimidin- 4-amine | ¹H NMR (400 MHz, CHLOROFORM-d) δ 7.51 (ddd, J = 9.8, 8.3, 1.9 Hz, 4H), 7.30- 7.24 (m, 2H), 6.98 (dd, J = 8.8, 2.0 Hz, 2H), 6.48 (q, J = 1.3 Hz, 1H), 4.83 (s, 1H), 3.85 (d, J = 1.7 Hz, 3H), 3.70 (qd, J = 6.6, 1.8 Hz, 2H), 2.82 (tt, J = 9.3, 6.4 Hz, 4H), |
| | | | | | | | | 2.43 (q, J = 1.3 Hz, 3H), 2.06 |
| | | | | | | | | (quin, J = 6.9 Hz, 2H), 1.34 (td, J = |
| | | | | | | | | 7.6, 1.8 Hz, 3H); LCMS: ESI |
| | | | | | | | | [M + H]⁺ = 418.2 |
| 275 | 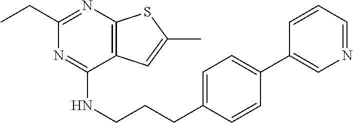 [SMILES: CCc1nc(NCCCc2ccc(-c3cccnc3)cc2)c2cc(C)sc2n1] | KH 4007 | Method A 2 d (80° C.) | 86% | Cs₂CO₃ 1.5 h (80° C.) | 37% | 2-ethyl-6- methyl-N-(3- (4-(pyridin-3- yl)phenyl)propyl) thieno[2,3- d]pyrimidin-4- amine | ¹H NMR (400 MHz, CHLOROFORM-d) δ 8.84 (s, 1H), 8.58 (d, J = 4.7 Hz, 1H), 7.86 (dt, J = 8.0, 1.9 Hz, 1H), 7.55-7.49 (m, 2H), 7.40-7.30 (m, 3H), 6.56 (t, J = 1.6 Hz, 1H), 4.88 (s, 1H), 3.70 (q, J = 6.6 Hz, 2H), 2.83 (dtd, J = 10.3, 7.7, 2.1 |
| | | | | | | | | Hz, 4H), 2.46 (d, J = 1.4 Hz, 3H), |
| | | | | | | | | 2.07 (quin, J = 7.2 Hz, 2H), 1.34 |
| | | | | | | | | (td, J = 7.6, 1.5 Hz, 3H); LCMS: |
| | | | | | | | | ESI [M + H]⁺ = 389.2 |
| 276 | 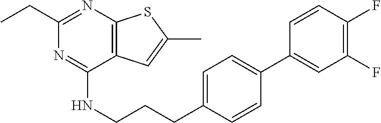 [SMILES: CCc1nc(NCCCc2ccc(-c3ccc(F)c(F)c3)cc2)c2cc(C)sc2n1] | KH 4007 | Method A 2 d (80° C.) | 86% | Cs₂CO₃ 1.5 h (80° C.) | 56% | N-(3-(3′,4′- difluoro-[1,1′- biphenyl]-4- yl)propyl)-2- ethyl-6- methylthieno[2, 3-d]pyrimidin- 4-amine | ¹H NMR (400 MHz, CHLOROFORM-d) δ 7.45 (dd, J = 8.2, 2.1 Hz, 2H), 7.42-7.15 (m, 5H), 6.54 (dd, J = 2.4, 1.3 Hz, 1H), 4.85 (s, 1H), 3.70 (qd, J = 6.8, 2.0 Hz, 2H), 2.82 (qd, J = 9.1, 8.4, 6.5 Hz, 4H), 2.49-2.44 (m, 3H), 2.12-2.00 (m, 2H), 1.34 |
| | | | | | | | | (td, J = 7.6, 2.1 Hz, 3H); LCMS: |
| | | | | | | | | ESI [M + H]⁺ = 424.2 |
| 277 | 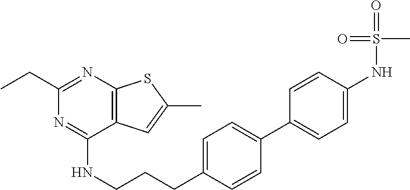 [SMILES: CCc1nc(NCCCc2ccc(-c3ccc(NS(C)(=O)=O)cc3)cc2)c2cc(C)sc2n1] | KH 4007 | Method A 2 d (80° C.) | 86% | Cs₂CO₃ 2 h (80° C.) | 26% | N-(4′-(3-((2- ethyl-6- methylthieno[2, 3-d]pyrimidin-4- yl)amino)propyl)- [1,1′- biphenyl]-4- yl)methanesul- fonamide | ¹H NMR (400 MHz, CHLOROFORM-d) δ 7.61-7.53 (m, 2H), 7.53-7.46 (m, 2H), 7.30 (dt, J = 8.7, 2.3 Hz, 4H), 6.60 (s, 1H), 6.57-6.51 (m, 1H), 4.89 (s, 1H), 3.70 (q, J = 6.6 Hz, 2H), 3.05 (d, J = 2.2 Hz, 3H), 2.84- 2.76 (m, 4H), 2.48-2.43 (m, 3H), 2.12-1.98 (m, 2H), 1.39-1.30 (m, 3H); LCMS: ESI [M + H]⁺ = 481.2 |
| 278 | 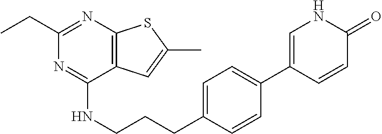 [SMILES: CCc1nc(NCCCc2ccc(-c3ccc(=O)[nH]c3)cc2)c2cc(C)sc2n1] | KH 4007 | Method A 2 d (80° C.) | 86% | Cs₂CO₃ 2 h (80° C.) | 20% | 5-(4-(3-((2- ethyl-6- methylthieno[2, 3-d]pyrimidin-4- yl)amino)propyl) phenyl)pyridin- 2(1H)-one | ¹H NMR (400 MHz, CHLOROFORM-d) δ 7.77 (dt, J = 9.5, 2.4 Hz, 1H), 7.59 (d, J = 2.5 Hz, 1H), 7.35 (dd, J = 8.2, 2.1 Hz, 2H), 7.29 (d, J = 2.1 Hz, 2H), 6.70 (dd, J = 9.5, 2.1 Hz, 1H), 6.58 (dd, J = 2.5, 1.3 Hz, 1H), 4.90 (s, 1H), 3.74-3.64 (m, 2H), |
| | | | | | | | | 2.88-2.74 (m, 4H), 2.52-2.47 |
| | | | | | | | | (m, 3H), 2.05 (quin, J = 6.9 Hz, |
| | | | | | | | | 2H), 1.34 (td, J = 7.6, 2.1 Hz, |
| | | | | | | | | 3H); LCMS: ESI [M + H]⁺ = 405.2 |
| 279 | 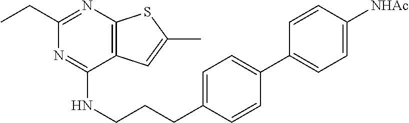 [SMILES: CCc1nc(NCCCc2ccc(-c3ccc(NC(C)=O)cc3)cc2)c2cc(C)sc2n1] | KH 4007 | Method A 2 d (80° C.) | 86% | Cs₂CO₃ 2 h (80° C.) | 42% | N-(4′-(3-((2- ethyl-6- methylthieno[2, 3-d]pyrimidin-4- yl)amino)propyl)- [1,1′- biphenyl]-4- yl)acetamide | ¹H NMR (400 MHz, CHLOROFORM-d) δ 7.61-7.46 (m, 6H), 7.37 (s, 1H), 7.31-7.27 (m, 2H), 6.50 (d, J = 2.3 Hz, 1H), 4.86 (s, 1H), 3.75-3.65 (m, 2H), 2.82 (qd, J = 8.7, 8.2, 6.1 Hz, 4H), 2.44 (d, J = 1.9 Hz, 3H), 2.20 (d, J = 2.0 Hz, 3H), 2.12- |
| | | | | | | | | 1.98 (m, 2H), 1.34 (td, J = 7.6, |
| | | | | | | | | 2.1 Hz, 3H); LCMS: ESI |
| | | | | | | | | [M + H]⁺ = 445.2 |
| 280 | 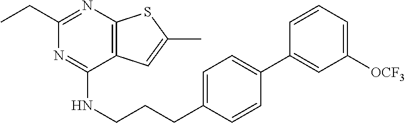 [SMILES: CCc1nc(NCCCc2ccc(-c3cccc(OC(F)(F)F)c3)cc2)c2cc(C)sc2n1] | KH 4007 | Method A 2 d (80° C.) | 86% | Cs₂CO₃ 2 h (80° C.) | 61% | 2-ethyl-6- methyl-N-(3-(3′- (trifluoromethoxy)- [1,1′- yl)propyl)thieno [2,3- d]pyrimidin-4- amine | ¹H NMR (400 MHz, CHLOROFORM-d) δ 7.55-7.47 (m, 3H), 7.47-7.40 (m, 2H), 7.31 (d, J = 7.8 Hz, 2H), 7.19 (ddt, J = 7.9, 2.4, 1.2 Hz, 1H), 6.56-6.51 (m, 1H), 4.90 (s, 1H), 3.70 (q, J = 6.5 Hz, 2H), 2.89-2.75 (m, 4H), 2.45 (d, J = 1.2 Hz, 3H), 2.07 |
| | | | | | | | | (quin, J = 7.1 Hz, 2H), 1.35 (td, J = |
| | | | | | | | | 7.6, 1.0 Hz, 3H); LCMS: ESI |
| | | | | | | | | [M + H]⁺ = 472.2 |
| 281 | 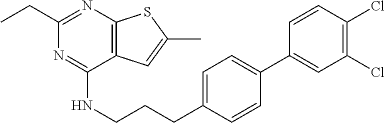 [SMILES: CCc1nc(NCCCc2ccc(-c3ccc(Cl)c(Cl)c3)cc2)c2cc(C)sc2n1] | KH 4007 | Method A 2 d (80° C.) | 86% | Cs₂CO₃ 2 h (80° C.) | 53% | N-(3-(3′,4′- dichloro-[1,1′- biphenyl]-4- yl)propyl)-2- ethyl-6- methylthieno[2, 3-d]pyrimidin- 4-amine | ¹H NMR (400 MHz, CHLOROFORM-d) δ 7.65 (d, J = 2.0 Hz, 1H), 7.48 (td, J = 8.5, 1.9 Hz, 3H), 7.40 (dt, J = 8.4, 2.0 Hz, 1H), 7.33-7.27 (m, 2H), 6.54 (quin, J = 1.2 Hz, 1H), 4.90 (s, 1H), 3.69 (q, J = 6.8, 6.3 Hz, 2H), 2.88-2.76 (m, 4H), 2.49-2.44 |
| | | | | | | | | (m, 3H), 2.06 (quin, J = 7.2 Hz, |
| | | | | | | | | 2H), 1.39-1.30 (m, 3H); LCMS: |
| | | | | | | | | ESI [M + H]⁺ = 456.1 |
| 282 | 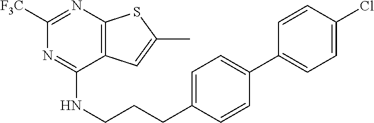 [SMILES: Cc1cc2c(NCCCc3ccc(-c4ccc(Cl)cc4)cc3)nc(C(F)(F)F)nc2s1] | KH 6080 | Method A 1 d (60° C.) | 93% | Cs₂CO₃ 2 h (80° C.) | 68% | N-(3-(4′- chloro-[1,1′- biphenyl]-4- yl)propyl)-6- methyl-2- (trifluoromethyl) thieno[2,3- d]pyrimidin-4- | ¹H NMR (400 MHz, CHLOROFORM-d) δ 7.53-7.47 (m, 4H), 7.43-7.37 (m, 2H), 7.33- 7.27 (m, 2H), 6.57 (d, J = 1.3 Hz, 1H), 5.13 (t, J = 5.6 Hz, 1H), 3.73 (q, J = 6.6 Hz, 2H), 2.81 (t, J = 7.3 Hz, 2H), 2.48 (d, J = 1.2 Hz, 3H), 2.08 (quin, J = 7.0 Hz, |
| | | | | | | | amine | 2H); LCMS: ESI [M + H]⁺ = |
| | | | | | | | | 462.1 |
| 283 | 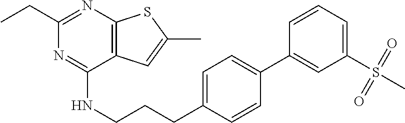 [SMILES: CCc1nc(NCCCc2ccc(-c3cccc(S(C)(=O)=O)c3)cc2)c2cc(C)sc2n1] | KH 4007 | Method A 2 d (80° C.) | 86% | Cs₂CO₃ 2 h (80° C.) | 58% | 2-ethyl-6- methyl-N-(3-(3′- (methylsulfonyl)- [1,1′-biphenyl]-4- yl)propyl)thieno [2,3-d]pyrimidin-4- amine | ¹H NMR (400 MHz, CHLOROFORM-d) δ 8.15 (t, J = 1.9 Hz, 1H), 7.87 (ddt, J = 20.3, 7.8, 1.4 Hz, 2H), 7.63 (t, J = 7.8 Hz, 1H), 7.57-7.51 (m, 2H), 7.36-7.31 (m, 2H), 6.60 (d, J = 1.4 Hz, 1H), 3.70 (q, J = 6.6 Hz, 2H), 3.10 (s, 3H), 2.83 (qd, J = |
| | | | | | | | | 7.6, 5.5 Hz, 4H), 2.47 (d, J = 1.2 |
| | | | | | | | | Hz, 3H), 2.08 (q, J = 7.2 Hz, 2H), |
| | | | | | | | | 1.34 (t, J = 7.6 Hz, 3H); LCMS: |
| | | | | | | | | ESI [M + H]⁺ = 466.2 |
| 284 | 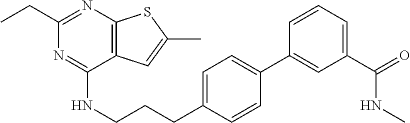 [SMILES: CCc1nc(NCCCc2ccc(-c3cccc(C(=O)NC)c3)cc2)c2cc(C)sc2n1] | KH 4007 | Method A 2 d (80° C.) | 86% | Cs₂CO₃ 2 h (80° C.) | 59% | 4′-(3-((2-ethyl-6- methylthieno[2, 3-d]pyrimidin-4- yl)amino)propyl) N-methyl- [1,1′-biphenyl]- 3-carboxamide | ¹H NMR (400 MHz, CHLOROFORM-d) δ 7.98 (q, J = 1.8 Hz, 1H), 7.69 (dt, J = 7.8, 1.7 Hz, 2H), 7.57-7.51 (m, 2H), 7.48 (td, J = 7.7, 1.6 Hz, 1H), 7.32-7.26 (m, 2H), 6.56 (t, J = 1.5 Hz, 1H), 6.31 (s, 1H), 5.02 (s, 1H), 3.69 (dt, J = 8.5, 6.1 Hz, |
| | | | | | | | | 2H), 3.04 (dd, J = 4.9, 1.6 Hz, |
| | | | | | | | | 3H), 2.82 (tt, J = 8.1, 6.3 Hz, 4H), |
| | | | | | | | | 2.43 (t, J = 1.5 Hz, 3H), 2.12- |
| | | | | | | | | 2.00 (m, 2H), 1.34 (td, J = 7.6, |
| | | | | | | | | 1.6 Hz, 3H); LCMS: ESI |
| | | | | | | | | [M + H]⁺ = 445.2 |
| 285 | 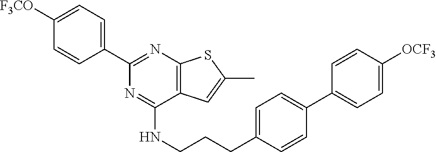 [SMILES: Cc1cc2c(NCCCc3ccc(-c4ccc(OC(F)(F)F)cc4)cc3)nc(-c3ccc(OC(F)(F)F)cc3)nc2s1] | 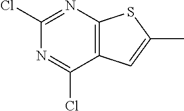 purchased | Method A 2 h (25° C.) | 89% | Cs₂CO₃ 1.5 h (65° C.) | 82% | 6-methyl-N-(3-(4′- (trifluoromethoxy)- [1,1′- biphenyl]-4- yl)propyl)-2-(4- (trifluoromethoxy) phenyl)thieno [2,3-d]pyrimidin-4- amine | ¹H NMR (400 MHz, CHLOROFORM-d) δ 8.45-8.37 (m, 2H), 7.59-7.51 (m, 2H), 7.50- 7.44 (m, 2H), 7.31-7.18 (m, 6H), 6.55 (d, J = 1.4 Hz, 1H), 4.90 (s, 1H), 3.76 (q, J = 6.5 Hz, 2H), 2.81 (t, J = 7.3 Hz, 2H), 2.45 (s, 3H), 2.09 (quin, J = 7.1 Hz, 2H); LCMS: ESI [M + H]⁺ = 604.2 |
| 286 | 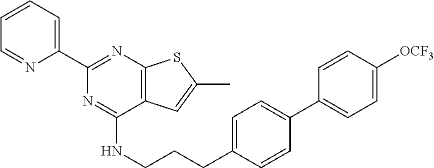 [SMILES: Cc1cc2c(NCCCc3ccc(-c4ccc(OC(F)(F)F)cc4)cc3)nc(-c3ccccn3)nc2s1] | KH 4036 | Method A 1 d (60° C.) | 75% | Cs₂CO₃ 1.5 h (80° C.) | 25% | 6-methyl-2- (pyridin-2-yl)- N-(3-(4′- (trifluoromethoxy)- [1,1′- biphenyl]-4- yl)propyl)thieno [2,3- d]pyrimidin-4- amine | ¹H NMR (400 MHz, CHLOROFORM-d) δ 8.82 (dd, J = 4.8, 1.0 Hz, 1H), 8.45 (dd, J = 8.0, 1.1 Hz, 1H), 7.77 (d, J = 1.8 Hz, 1H), 7.61-7.53 (m, 2H), 7.53-7.46 (m, 2H), 7.37-7.27 (m, 5H), 6.65 (d, J = 1.5 Hz, 1H), 5.18 (s, 1H), 3.84 (d, J = 6.3 Hz, 2H), 2.85 (t, J = 7.3 Hz, 2H), 2.48 (d, J = 1.2 Hz, 3H), 2.25-2.04 |
| | | | | | | | | (m, 2H); LCMS: ESI [M + H]⁺ = |
| | | | | | | | | 521.2 |
| 287 | 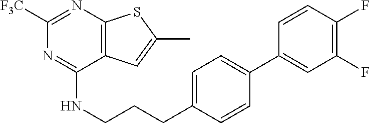 [SMILES: Cc1cc2c(NCCCc3ccc(-c4ccc(F)c(F)c4)cc3)nc(C(F)(F)F)nc2s1] | KH 6080 | Method A l d (60° C.) | 93% | Cs₂CO₃ 1.5 h (80° C.) | 57% | N-(3-(3′,4′- difluoro-[1,1′- biphenyl]-4- yl)propyl)-6- methyl-2- (trifluoromethyl) thieno[2,3- d]pyrimidin-4- | ¹H NMR (400 MHz, CHLOROFORM-d) δ 7.47-7.28 (m, 5H), 6.93 (dtd, J = 13.2, 8.6, 2.7 Hz, 2H), 6.63-6.58 (m, 1H), 5.12 (s, 1H), 3.74 (dp, J = 10.1, 3.2 Hz, 2H), 2.81 (td, J = 7.3, 3.5 Hz, 2H), 2.55-2.50 (m, 3H), 2.09 (ddt, J = 10.8, 7.1, 3.8 Hz, |
| | | | | | | | amine | 2H); LCMS: ESI [M + H]⁺ = 464.1 |
| 288 | 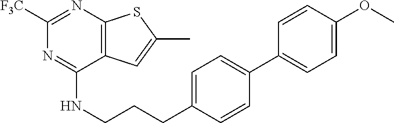 [SMILES: COc1ccc(-c2ccc(CCCNc3nc(C(F)(F)F)nc4sc(C)cc34)cc2)cc1] | KH 6080 | Method A 1 d (60° C.) | 93% | Cs₂CO₃ 1.5 h (80° C.) | 77% | N-(3-(4′- methoxy-[1,1′- biphenyl]-4- yl)propyl)-6- methyl-2- (trifluoromethyl) thieno[2,3- d]pyrimidin-4- | ¹H NMR (400 MHz, CHLOROFORM-d) δ 7.56-7.47 (m, 4H), 7.31-7.24 (m, 2H), 7.02- 6.94 (m, 2H), 6.51-6.46 (m, 1H), 5.04 (s, 1H), 3.86 (s, 3H), 3.74 (q, J = 6.4 Hz, 2H), 2.81 (t, J = 7.2 Hz, 2H), 2.44 (d, J = 1.2 Hz, 3H), 2.09 (quin, J = 6.9 Hz, |
| | | | | | | | amine | 2H); LCMS: ESI [M + H]⁺ = 458.2 |
| 289 | 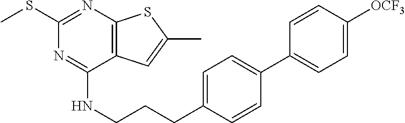 [SMILES: CSc1nc(NCCCc2ccc(-c3ccc(OC(F)(F)F)cc3)cc2)c2cc(C)sc2n1] | KH 5002 | Method A 1 d (60° C.) | 86% | Cs₂CO₃ 1.5 h (80° C.) | 44% | 6-methyl-2- (methylthio)-N- (3-(4′- (trifluoromethoxy)- [1,1′- biphenyl]-4- yl)propyl)thieno [2,3-d]pyrimidin-4- | ¹H NMR (400 MHz, CHLOROFORM-d) δ 7.58 (d, J = 8.8 Hz, 2H), 7.50 (d, J = 8.2 Hz, 2H), 7.34-7.26 (m, 4H), 6.47 (d, J = 1.4 Hz, 1H), 4.98 (s, 1H), 3.68 (d, J = 6.3 Hz, 2H), 2.79 (t, J = 7.3 Hz, 2H), 2.54 (s, 3H), 2.41 (d, J = 1.2 Hz, 3H), 2.11-1.99 |
| | | | | | | | amine | (m, 2H); LCMS: ESI |
| | | | | | | | | [M + H]⁺ = 490.1 |
| 290 | 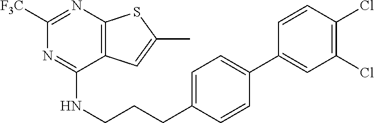 [SMILES: Cc1cc2c(NCCCc3ccc(-c4ccc(Cl)c(Cl)c4)cc3)nc(C(F)(F)F)nc2s1] | KH 6080 | Method A 1 d (60° C.) | 93% | Cs₂CO₃ 2 h (80° C.) | 72% | N-(3-(3′,4′- dichloro-[1,1′- biphenyl]-4- yl)propyl)-6- methyl-2- (trifluoromethyl) thieno[2,3- d]pyrimidin-4- | ¹H NMR (400 MHz, CHLOROFORM-d) δ 7.64 (d, J = 2.1 Hz, 1H), 7.48 (dd, J = 9.7, 7.6 Hz, 3H), 7.39 (dd, J = 8.3, 2.2 Hz, 1H), 7.34-7.28 (m, 2H), 6.59 (d, J = 1.4 Hz, 1H), 5.11 (s, 1H), 3.73 (q, J = 6.6 Hz, 2H), 2.81 (t, J = 7.3 Hz, 2H), 2.51 (d, J = 1.1 |
| | | | | | | | amine | Hz, 3H), 2.09 (t, J = 7.1 Hz, 2H); |
| | | | | | | | | LCMS: ESI [M + H]⁺ = 496.1 |
| 291 | 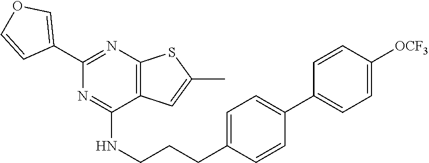 [SMILES: Cc1cc2c(NCCCc3ccc(-c4ccc(OC(F)(F)F)cc4)cc3)nc(-c3ccoc3)nc2s1] | KH 6067 | Method A 1 d (60° C.) | 70% | Cs₂CO₃ 2 h (80° C.) | 57% | 2-(furan-3-yl)- 6-methyl-N-(3-(4′- (trifluoromethoxy)- [1,1′-biphenyl]-4- yl)propyl)thieno [2,3- d]pyrimidin-4- amine | ¹H NMR (400 MHz, DMSO-d₆) δ 8.18 (s, 1H), 7.74-7.86 (m, 3H), 7.68 (s, 1H), 7.58-7.63 (m, J = 7.95 Hz, 2H), 7.41-7.46 (m, J = 8.44 Hz, 2H), 7.36 (d, J = 7.95 Hz, 2H), 7.24 (s, 1H), 6.92 (s, 1H), 3.58 (q, J = 6.56 Hz, 2H), 3.31 (s, 3H), 2.75 (t, J = 7.34 Hz, 2H), 1.98 (t, J = 7.03 Hz, 2H); LCMS: ESI [M + H]⁺ = 510.2 |
| 292 | 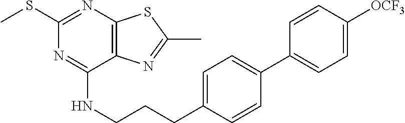 [SMILES: CSc1nc(NCCCc2ccc(-c3ccc(OC(F)(F)F)cc3)cc2)c2nc(C)sc2n1] | LM 13029 | Method A 2 h (60° C.) | 99% | Cs₂CO₃ 1.5 h (80° C.) | 46% | 2-methyl-5- (methylthio)-N- (3-(4′- (trifluoromethoxy)- [1,1′-biphenyl]-4- yl)propyl)thiazolo [5,4-d]pyrimidin-7- amine | ¹H NMR (400 MHz, METHANOL-d₄) δ 7.69 (d, J = 8.68 Hz, 2H), 7.53 (d, J = 7.95 Hz, 2H), 7.33 (t, J = 8.31 Hz, 4H), 3.65 (t, J = 6.66 Hz, 2H), 2.79 (t, J = 7.40 Hz, 2H), 2.72 (s, 3H), 2.47 (s, 3H), 2.00-2.13 (m, 2H); LCMS: ESI [M + H]⁺ = 491.2 |
| 293 | 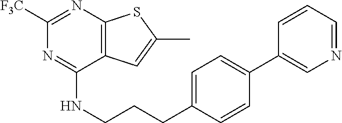 [SMILES: Cc1cc2c(NCCCc3ccc(-c4cccnc4)cc3)nc(C(F)(F)F)nc2s1] | KH 6080 | Method A 1 d (60° C.) | 93% | Cs₂CO₃ 1.5 h (80° C.) | 47% | 6-methyl-N-(3- (4-(pyridin-3- yl)phenyl)propyl)- 2-(trifluoromethyl) thieno [2,3- d]pyrimidin-4- amine | ¹H NMR (400 MHz, CHLOROFORM-d) δ 8.84 (s, 1H), 8.58 (d, J = 4.9 Hz, 1H), 7.87 (dt, J = 7.9, 1.8 Hz, 1H), 7.52 (dd, J = 8.2, 1.5 Hz, 2H), 7.40-7.32 (m, 3H), 6.64 (t, J = 1.3 Hz, 1H), 5.23 (s, 1H), 3.79- 3.69 (m, 2H), 2.82 (t, J = 7.4 Hz, |
| | | | | | | | | 2H), 2.51 (d, J = 1.3 Hz, 3H), |
| | | | | | | | | 2.10 (q, J = 7.1 Hz, 2H); LCMS: |
| | | | | | | | | ESI [M + H]⁺ = 429.2 |
| 294 | 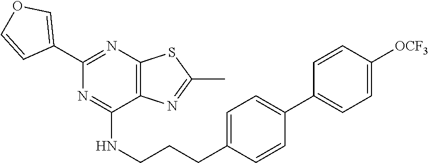 [SMILES: Cc1nc2c(NCCCc3ccc(-c4ccc(OC(F)(F)F)cc4)cc3)nc(-c3ccoc3)nc2s1] | LM 13028 | Method A 1 d (60° C.) | 40% | Cs₂CO₃ 2 h (80° C.) | 37% | 5-(furan-3-yl)- 2-methyl-N-(3-(4′- (trifluoromethoxy)- [1,1′-biphenyl]-4- yl)propyl)thiazolo [5,4-d]pyrimidin-7- amine | ¹H NMR (400 MHz, CHLOROFORM-d) δ 8.14 (br. s., 1H), 7.56-7.63 (m, 2H), 7.42- 7.52 (m, 3H), 7.31 (dd, J = 1.57, 8.61 Hz, 4H), 7.01 (br. s., 1H), 5.97 (br. s., 1H), 3.75 (d, J = 6.26 Hz, 2H), 2.82 (t, J = 7.24 Hz, 2H), 2.74-2.77 (m, 3H), 2.06- 2.15 (m, 2H); LCMS: ESI [M + H]⁺ = 511.2 |
| 295 | 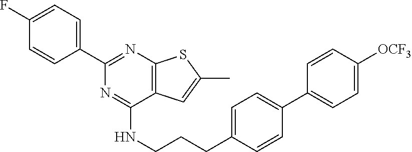 [SMILES: Cc1cc2c(NCCCc3ccc(-c4ccc(OC(F)(F)F)cc4)cc3)nc(-c3ccc(F)cc3)nc2s1] | KH 5015 | Method A 1 d (60° C.) 1 d (80° C.) | 86% | Cs₂CO₃ 2 h (80° C.) | 60% | 2-(4- fluorophenyl)- 6-methyl-N-(3-(4′- (trifluoromethoxy)- [1,1′-biphenyl]-4- yl)propyl)thieno [2,3-d]pyrimidin-4- amine | ¹H NMR (400 MHz, CHLOROFORM-d) δ 8.36-8.47 (m, 2H), 7.55 (dd, J = 2.35, 8.61 Hz, 2H), 7.44-7.50 (m, 2H), 7.22-7.30 (m, 4H), 7.00-7.11 (m, 2H), 6.55 (s, 1H), 4.89 (br. s., 1H), 3.78 (q, J = 6.39 Hz, 2H), 2.82 (t, J = 7.24 Hz, 2H), 2.45 (s, 3H), 2.11 (t, J = 7.04 Hz, 2H); |
| | | | | | | | | LCMS: ESI [M + H]⁺ = 538.2 |
| 296 | 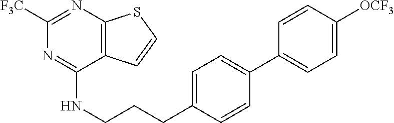 [SMILES: FC(F)(F)Oc1ccc(-c2ccc(CCCNc3nc(C(F)(F)F)nc4sccc34)cc2)cc1] | LM 13024 F1 | Method A 3 d (90° C.) | 61% | Cs₂CO₃ 2 h (80° C.) | 48% | N-(3-(4′- (trifluoromethoxy)- [1,1′-biphenyl]-4- yl)propyl)-2- (trifluoromethyl) thieno[2,3- d]pyrimidin-4- amine | ¹H NMR (400 MHz, CHLOROFORM-d) δ 7.58 (d, J = 8.22 Hz, 2H), 7.50 (d, J = 7.83 Hz, 2H), 7.39 (d, J = 5.87 Hz, 1H), 7.31 (dd, J = 8.22, 12.13 Hz, 4H), 7.00 (d, J = 5.87 Hz, 1H), 5.33 (br. s., 1H), 3.78 (q, J = 6.65 Hz, 2H), 2.84 (t, J = 7.24 Hz, |
| | | | | | | | | 2H), 2.06-2.21 (m, 2H); LCMS: |
| | | | | | | | | ESI [M + H]⁺ = 498.2 |
| 297 | 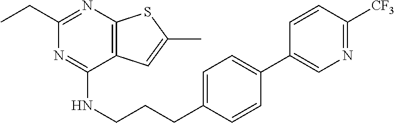 [SMILES: CCc1nc(NCCCc2ccc(-c3ccc(C(F)(F)F)nc3)cc2)c2cc(C)sc2n1] | KH 4007 | Method A 2 d (80° C.) | 86% | Cs₂CO₃ 2 h (80° C.) | 61% | 2-ethyl-6- methyl-N-(3- (4-(6- (trifluoromethyl) pyridin-3- yl)phenyl)propyl) thieno[2,3- d]pyrimidin-4- | ¹H NMR (400 MHz, CHLOROFORM-d) δ 8.95 (d, J = 6.65 Hz, 1H), 8.03 (d, J = 7.83 Hz, 1H), 7.77 (t, J = 8.80 Hz, 1H), 7.50-7.60 (m, 2H), 7.33- 7.44 (m, 2H), 6.64 (br. s., 1H), 3.72 (br. s., 2H), 2.75-2.94 (m, 4H), 2.51 (d, J = 8.61 Hz, 3H), |
| | | | | | | | amine | 2.10 (d, J = 6.65 Hz, 2H), 1.32- |
| | | | | | | | | 1.47 (m, 3H); LCMS: ESI |
| | | | | | | | | [M + H]⁺ = 457.2 |
| 298 | 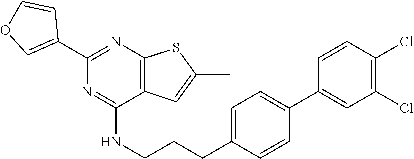 [SMILES: Cc1cc2c(NCCCc3ccc(-c4ccc(Cl)c(Cl)c4)cc3)nc(-c3ccoc3)nc2s1] | KH 6067 | Method A 1 d (60° C.) | 70% | Cs₂CO₃ 2 h (80° C.) | 18% | N-(3-(3′,4′- dichloro-[1,1′- biphenyl]-4- yl)propyl)-2- (furan-3-yl)-6- methylthieno[2, 3-d]pyrimidin- 4-amine | ¹H NMR (400 MHz, CHLOROFORM-d) δ 8.15 (br. s., 1H), 7.68 (d, J = 1.96 Hz, 1H), 7.38-7.57 (m, 5H), 7.33 (d, J = 7.95 Hz, 2H), 7.06 (br. s., 1H), 6.60 (br. s., 1H), 4.93 (br. s., 1H), 3.76 (q, J = 6.64 Hz, 2H), 2.84 (t, J = 6.79 Hz, 2H), 2.51 (s, 3H), 2.12 (t, J = 6.85 Hz, 2H); LCMS: ESI [M + H]⁺ = 494.1 |
| 299 | 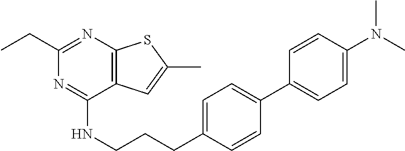 | KH 4007 | Method A 2 d (80° C.) | 86% | Cs₂CO₃ 2 h (80° C.) | 61% | N-(3-(4′- (dimethylamino)- [1,1′-biphenyl]-4- yl)propyl)-2- ethyl-6- methylthieno[2, 3-d]pyrimidin- 4-amine | ¹H NMR (400 MHz, CHLOROFORM-d) δ 7.51 (dd, J = 2.02, 8.38 Hz, 4H), 7.25-7.28 (m, 2H), 6.82 (d, J = 8.80 Hz, 2H), 6.49 (s, 1H), 3.71 (q, J = 6.60 Hz, 2H), 3.00 (s, 6H), 2.75- 2.90 (m, 4H), 2.43 (s, 3H), 2.07 (t, J = 7.03 Hz, 2H), 1.36 (t, J = 7.58 Hz, 3H); LCMS: ESI |
| | | | | | | | | [M + H]⁺ = 431.3 |
| 300 | 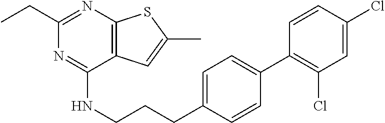 | KH 4007 | Method A 2 d (80° C.) | 86% | Cs₂CO₃ 2 h (80° C.) | 58% | N-(3-(2′,4′- dichloro-[1,1′- biphenyl]-4- yl)propyl)-2- ethyl-6- methylthieno[2, 3-d]pyrimidin- 4-amine | ¹H NMR (400 MHz, CHLOROFORM-d) δ 7.50 (d, J = 1.83 Hz, 1H), 7.34-7.40 (m, 2H), 7.22-7.32 (m, 4H), 6.65 (s, 1H), 3.72 (d, J = 6.24 Hz, 2H), 2.84 (quin, J = 7.86 Hz, 4H), 2.52 (s, 3H), 2.09 (t, J = 7.27 Hz, 2H), 1.36 (t, J = 7.58 Hz, 3H); LCMS: |
| | | | | | | | | ESI [M + H]⁺ = 456.1 |
| 301 | 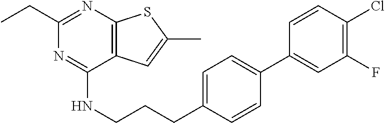 [SMILES: CCc1nc(NCCCc2ccc(-c3ccc(Cl)c(F)c3)cc2)c2cc(C)sc2n1] | KH 4007 | Method A 2 d (80° C.) | 86% | Cs₂CO₃ 2 h (80° C.) | 29% | N-(3-(4′- chloro-3′- fluoro-[1,1′- biphenyl]-4- yl)propyl)-2- ethyl-6- methylthieno[2, 3-d]pyrimidin- | ¹H NMR (400 MHz, CHLOROFORM-d) δ 7.41-7.54 (m, 3H), 7.29-7.38 (m, 4H), 6.60 (br. s., 1H), 3.71 (q, J = 6.68 Hz, 2H), 2.78-2.91 (m, 4H), 2.48 (s, 3H), 2.09 (t, J = 7.21 Hz, 2H), 1.36 (t, J = 7.52 Hz, 3H); LCMS: ESI [M + H]⁺ = 440.2 |
| | | | | | | | 4-amine | |
| 302 | 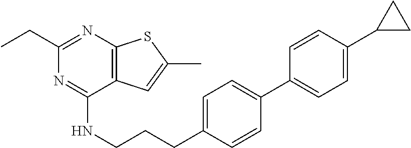 [SMILES: CCc1nc(NCCCc2ccc(-c3ccc(C4CC4)cc3)cc2)c2cc(C)sc2n1] | KH 4007 | Method A 2 d (80° C.) | 86% | Cs₂CO₃ 2 h (80° C.) | 18% | N-(3-(4′- cyclopropyl- [1,1′-biphenyl]- 4-yl)propyl)-2- ethyl-6- methylthieno[2, 3-d]pyrimidin- 4-amine | ¹H NMR (400 MHz, CHLOROFORM-d) δ 7.51 (dd, J = 8.19, 14.06 Hz, 4H), 7.29 (d, J = 8.07 Hz, 2H), 7.15 (d, J = 8.31 Hz, 2H), 6.52 (s, 1H), 3.71 (q, J = 6.64 Hz, 2H), 2.76-2.91 (m, 4H), 2.44 (s, 3H), 2.02-2.15 (m, 2H), 1.87-1.98 (m, 1H), 1.36 (t, J = 7.58 Hz, 3H), 0.94-1.04 (m, |
| | | | | | | | | 2H), 0.70-0.78 (m, 2H); LCMS: |
| | | | | | | | | ESI [M + H]⁺ = 428.3 |
| 303 | 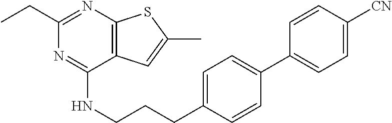 | KH 4007 | Method A 2 d (80° C.) | 86% | Cs₂CO₃ 2 h (80° C.) | 54% | 4′-(3-((2-ethyl-6- methylthieno[2, 3-d]pyrimidin-4- yl)amino)propyl)- [1,1′-biphenyl]-4- carbonitrile | ¹H NMR (400 MHz, CHLOROFORM-d) δ 7.64-7.78 (m, 4H), 7.53 (d, J = 8.07 Hz, 2H), 7.34 (d, J = 8.07 Hz, 2H), 6.60 (s, 1H), 3.71 (q, J = 6.68 Hz, 2H), 2.80-2.88 (m, 4H), 2.48 (s, 3H), 2.08 (quin, J = 7.18 Hz, 2H), 1.35 (t, J = 7.58 Hz, 3H); LCMS: |
| | | | | | | | | ESI [M + H]⁺ = 413.2 |
| 304 | 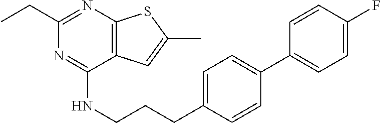 | KH 4007 | Method A 2 d (80° C.) | 86% | Cs₂CO₃ 2 h (80° C.) | 59% | 2-ethyl-N-(3- (4′-fluoro- [1,1′-biphenyl]- 4-yl)propyl)-6- methylthieno[2, 3-d]pyrimidin- 4-amine | ¹H NMR (400 MHz, CHLOROFORM-d) δ 7.43-7.59 (m, 4H), 7.24-7.35 (m, 2H), 7.13 (t, J = 8.62 Hz, 2H), 6.54 (s, 1H), 3.71 (q, J = 6.52 Hz, 2H), 2.76- 2.91 (m, 4H), 2.45 (s, 3H), 2.08 (quin, J = 7.09 Hz, 2H), 1.36 (t, J = 7.58 Hz, 3H); LCMS: ESI |
| | | | | | | | | [M + H]⁺ = 406.2 |
| 305 | 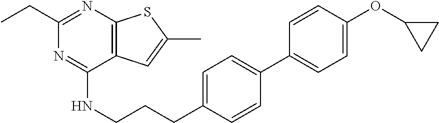 [SMILES: CCc1nc(NCCCc2ccc(-c3ccc(OC4CC4)cc3)cc2)c2cc(C)sc2n1] | KH 4007 | Method A 2d (80° C.) | 86% | Cs₂CO₃ 2 h (80° C.) | 61% | N-(3-(4′- cyclopropoxy- [1,1′-biphenyl]-4-yl) propyl)-2-ethyl-6- methylthieno[2, 3-d]pyrimidin- 4-amine | ¹H NMR (400 MHz, CHLOROFORM-d) δ 7.51 (dd, J = 6.54, 8.25 Hz, 4H), 7.29 (d, J = 8.07 Hz, 2H), 7.13 (d, J = 8.68 Hz, 2H), 6.51 (s, 1H), 4.93 (br. s., 1H), 3.79 (td, J = 4.54, 8.89 Hz, 1H), 3.71 (q, J = 6.60 Hz, 2H), 2.75-2.90 (m, 4H), 2.44 (s, 3H), |
| | | | | | | | | 2.07 (quin, J = 7.06 Hz, 2H), 1.36 |
| | | | | | | | | (t, J = 7.58 Hz, 3H), 0.78-0.85 |
| | | | | | | | | (m, 4H); LCMS: ESI [M + H]⁺ = |
| | | | | | | | | 444.3 |
| 306 | 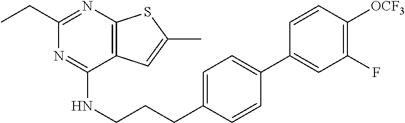 | KH 4007 | Method A 2 d (80° C.) | 86% | Cs₂CO₃ 2 h (80° C.) | 69% | 2-ethyl-N-(3- (3′-fluoro-4′- (trifluoromethoxy)- [1,1′-biphenyl]-4- yl)propyl)-6- methylthieno[2, 3-d]pyrimidin- 4-amine | ¹H NMR (400 MHz, CHLOROFORM-d) δ 7.48 (d, J = 8.19 Hz, 2H), 7.30-7.43 (m, 5H), 6.58 (s, 1H), 3.71 (q, J = 6.60 Hz, 2H), 2.84 (quin, J = 7.70 Hz, 4H), 2.47 (s, 3H), 2.08 (quin, J = 7.15 Hz, 2H), 1.36 (t, J = 7.58 Hz, 3H); LCMS: ESI [M + H]⁺ = |
| | | | | | | | | 490.2 |
| 307 | 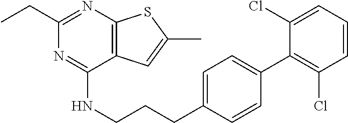 | KH 4007 | Method A 2 d (80° C.) | 86% | Cs₂CO₃ 3 h (80° C.) | 19% | N-(3-(2′,6′- dichloro-[1,1′- biphenyl]-4- yl)propyl)-2- ethyl-6- methylthieno[2, 3-d]pyrimidin- 4-amine | ¹H NMR (400 MHz, CHLOROFORM-d) δ 7.41 (d, J = 8.07 Hz, 2H), 7.33 (d, J = 8.07 Hz, 2H), 7.18-7.25 (m, 3H), 6.70 (s, 1H), 3.72 (q, J = 6.68 Hz, 2H), 2.80-2.92 (m, 4H), 2.54 (s, 3H), 2.12 (t, J = 7.34 Hz, 2H), 1.37 (t, J = 7.58 Hz, 3H); LCMS: |
| | | | | | | | | ESI [M + H]⁺ = 456.1 |
| 308 | 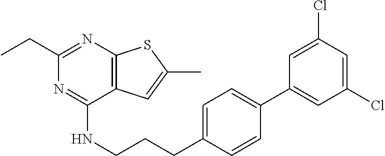 [SMILES: CCc1nc(NCCCc2ccc(-c3cc(Cl)cc(Cl)c3)cc2)c2cc(C)sc2n1] | KH 4007 | Method A 2 d (80° C.) | 86% | Cs₂CO₃ 2 h (80° C.) | 52% | N-(3-(3′,5′- dichloro-[1,1′- biphenyl]-4- yl)propyl)-2- ethyl-6- methylthieno[2, 3-d]pyrimidin- 4-amine | ¹H NMR (400 MHz, CHLOROFORM-d) δ 7.42-7.52 (m, 4H), 7.29-7.35 (m, 3H), 6.55 (s, 1H), 3.71 (q, J = 6.52 Hz, 2H), 2.84 (quin, J = 7.79 Hz, 4H), 2.48 (s, 3H), 2.08 (quin, J = 7.12 Hz, 2H), 1.36 (t, J = 7.58 Hz, 3H); LCMS: ESI [M + H]⁺ = 456.1 |
| 309 | 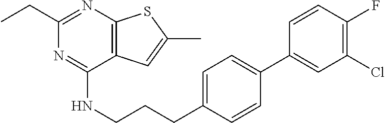 [SMILES: CCc1nc(NCCCc2ccc(-c3ccc(F)c(Cl)c3)cc2)c2cc(C)sc2n1] | KH 4007 | Method A 2 d (80° C.) | 86% | Cs₂CO₃ 2 h (80° C.) | 67% | N-(3-(3′- chloro-4′- fluoro-[1,1′- biphenyl]-4- yl)propyl)-2- ethyl-6- methylthieno[2, 3-d]pyrimidin- | ¹H NMR (400 MHz, CHLOROFORM-d) δ 7.60 (dd, J = 2.20, 6.97 Hz, 1H), 7.46 (d, J = 8.19 Hz, 2H), 7.42 (ddd, J = 2.26, 4.43, 6.39 Hz, 1H), 7.30 (d, J = 8.07 Hz, 2H), 7.20 (t, J = 8.68 Hz, 1H), 6.56 (s, 1H), 3.71 (q, J = 6.64 Hz, 2H), 2.77-2.89 (m, |
| | | | | | | | 4-amine | 4H), 2.47 (s, 3H), 1.99-2.12 (m, |
| | | | | | | | | 2H), 1.36 (t, J = 7.58 Hz, 3H); |
| | | | | | | | | LCMS: ESI [M + H]⁺ = 440.2 |
| 310 | 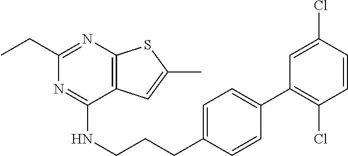 [SMILES: CCc1nc(NCCCc2ccc(-c3cc(Cl)ccc3Cl)cc2)c2cc(C)sc2n1] | KH 4007 | Method A 2 d (80° C.) | 86% | Cs₂CO₃ 2 h (80° C.) | 47% | N-(3-(2′,5′- dichloro-[1,1′- biphenyl]-4- yl)propyl)-2- ethyl-6- methylthieno[2, 3-d]pyrimidin- 4-amine | ¹H NMR (400 MHz, CHLOROFORM-d) δ 7.34-7.43 (m, 3H), 7.28-7.34 (m, 3H), 7.22- 7.27 (m, 1H), 6.64 (s, 1H), 3.72 (q, J = 6.68 Hz, 2H), 2.85 (quin, J = 7.67 Hz, 4H), 2.52 (s, 3H), 2.09 (quin, J = 7.18 Hz, 2H), 1.37 (t, J = 7.58 Hz, 3H); LCMS: ESI [M + H]⁺ = 456.1 |
| 311 | 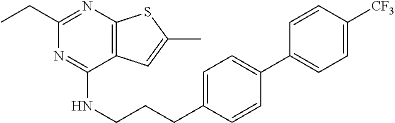 [SMILES: CCc1nc(NCCCc2ccc(-c3ccc(C(F)(F)F)cc3)cc2)c2cc(C)sc2n1] | KH 4007 | Method A 2 d (80° C.) | 86% | Cs₂CO₃ 2 h (80° C.) | 45% | 2-ethyl-6- methyl-N-(3-(4′- (trifluoromethyl)- [1,1′-biphenyl]-4- yl)propyl)thieno [2,3- d]pyrimidin-4- amine | ¹H NMR (400 MHz, METHANOL-d₄) δ 7.75-7.80 (m, 2H), 7.68-7.74 (m, 2H), 7.57 (d, J = 8.07 Hz, 2H), 7.34 (d, J = 8.07 Hz, 2H), 6.99 (s, 1H), 3.65 (t, J = 6.97 Hz, 2H), 2.70-2.83 (m, 4H), 2.51 (s, 3H), 2.08 (quin, J = 7.24 Hz, 2H), 1.32 (t, J = 7.64 |
| | | | | | | | | Hz, 3H); LCMS: ESI [M + H]⁺ = |
| | | | | | | | | 456.2 |
| 312 | 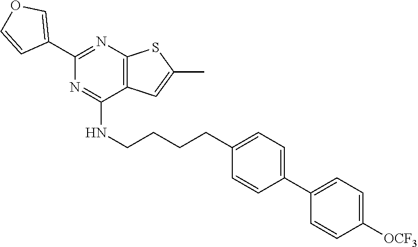 [SMILES: Cc1cc2c(NCCCCc3ccc(-c4ccc(OC(F)(F)F)cc4)cc3)nc(-c3ccoc3)nc2s1] | KH 6067 | Method A MW 1 h (140° C.) | 67% | Cs₂CO₃ 1 d (80° C.) | 70% | 2-(furan-3-yl)- 6-methyl-N-(4-(4′- (trifluoromethoxy)- [1,1′-biphenyl]-4- yl)butyl)thieno [2,3- d]pyrimidin-4- amine | ¹H NMR (400 MHz, METHANOL-d4) δ 6.59 (d, J = 0.86 Hz, 1H), 6.08 (d, J = 8.80 Hz, 2H), 5.97 (t, J = 1.71 Hz, 1H), 5.93 (d, J = 8.19 Hz, 2H), 5.75 (d, J = 8.07 Hz, 2H), 5.71 (d, J = 8.19 Hz, 2H), 5.50 (d, J = 1.22 Hz, 1H), 5.46 (d, J = 1.22 Hz, 1H), 2.05-2.16 (m, 2H), 1.13-1.24 (m, 2H), 0.99 (d, J = 1.10 Hz, 3H), 0.23 (td, J = 3.45, 6.79 Hz, 4H); LCMS: ESI [M + H]+ = 524.1 |
| 313 | 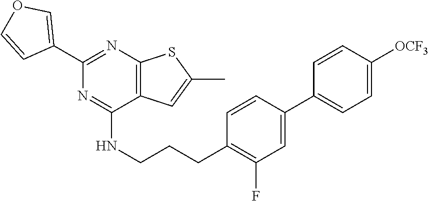 [SMILES: Cc1cc2c(NCCCc3ccc(-c4ccc(OC(F)(F)F)cc4)cc3F)nc(-c3ccoc3)nc2s1] | KH 6067 | Method A MW 1 h (140° C.) | 48% | Cs₂CO₃ 1 h (100° C.) | 63% | N-(3-(3-fluoro- (trifluoromethoxy)- [1,1′- biphenyl]-4-′- yl)propyl)-2- (furan-3-yl)-6- methylthieno[2, 3-d]pyrimidin- 4-amine | ¹H NMR (399 MHz, METHANOL-d4) δ 8.08 (s, 1H), 7.68 (d, J = 8.56 Hz, 2H), 7.51 (s, 1H), 7.29-7.39 (m, 5H), 7.03 (s, 1H), 6.95 (s, 1H), 3.70 (t, J = 7.01 Hz, 2H), 2.83 (t, J = 7.20 Hz, 2H), 2.53 (s, 3H), 2.08 (quin, J = 7.01 Hz, 2H); LCMS: ESI [M + H]+ = 528.1 |
| 314 | 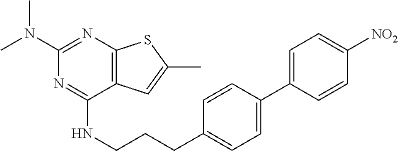 [SMILES: Cc1cc2c(NCCCc3ccc(-c4ccc([N+](=O)[O-])cc4)cc3)nc(N(C)C)nc2s1] | KH 5048 | Method A 1 d (80° C.), 1 d (110° C.) | 29% | Cs₂CO₃ 1 h (90° C.) | 7% | N2,N2,6- trimethyl-N4- (3-(4′-nitro- [1,1′-biphenyl]- 4-yl)propyl)thieno [2,3- d]pyrimidine- 2,4-diamine | ¹H NMR (399 MHz, METHANOL-d4) δ 8.26-8.34 (m, J = 8.56 Hz, 2H), 7.80-7.88 (m, J = 8.56 Hz, 2H), 7.59-7.67 (m, J = 7.79 Hz, 2H), 7.32-7.39 (m, J = 8.17 Hz, 2H), 6.78 (s, 1H), 3.57 (t, J = 7.20 Hz, 2H), 3.08 (s, 6H), 2.78 (t, J = 7.59 Hz, 2H), 2.40 (s, 3H), 2.04 (quin, J = |
| | | | | | | | | 7.20 Hz, 2H); LCMS: ESI |
| | | | | | | | | [M + H]+ = 448.2 |
| 315 | 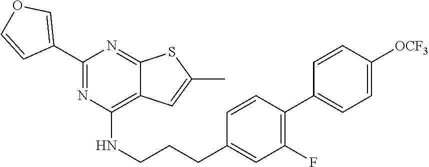 [SMILES: Cc1cc2c(NCCCc3ccc(-c4ccc(OC(F)(F)F)cc4)c(F)c3)nc(-c3ccoc3)nc2s1] | KH 6067 | Method A 2 d (70° C.) | 73% | Cs₂CO₃ MW 1 h (140° C.) | 17% | N-(3-(2-fluoro-4′- (trifluoromethoxy)- [1,1′- biphenyl]-4- yl)propyl)-2- (furan-3-yl)-6- methylthieno[2, 3-d]pyrimidin- 4-amine | ¹H NMR (399 MHz, METHANOL-d4) δ 8.08 (s, 1H), 7.59 (d, J = 8.56 Hz, 2H), 7.51 (s, 1H), 7.29-7.39 (m, 3H), 7.04- 7.14 (m, 2H), 6.97 (s, 1H), 7.01 (s, 1H), 3.68 (t, J = 7.01 Hz, 2H), 2.80 (t, J = 7.20 Hz, 2H), 2.52 (s, 3H), 2.08 (quin, J = 7.10 Hz, 2H); LCMS: ESI [M + H]+ = 528.1 |
| 316 | 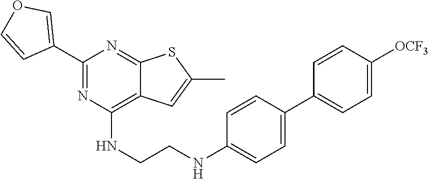 [SMILES: Cc1cc2c(NCCNc3ccc(-c4ccc(OC(F)(F)F)cc4)cc3)nc(-c3ccoc3)nc2s1] | KH 6067 | Method A MW 1 h (140° C.) | 39% | Cs₂CO₃ MW 1 h (140° C.) | 3% | N1-(2-(furan-3- yl)-6- methylthieno[2, 3-d]pyrimidin- 4-yl)-N2-(4′- (trifluoromethoxy)- [1,1′-biphenyl]-4- yl)ethane-1,2- diamine | ¹H NMR (399 MHz, METHANOL-d4) δ 8.19 (s, 1H), 7.58 (d, J = 8.56 Hz, 3H), 7.34- 7.42 (m, J = 7.79 Hz, 2H), 7.22- 7.30 (m, J = 8.56 Hz, 2H), 7.05 (br. s., 2H), 6.74 (d, J = 7.40 Hz, 2H), 3.88 (t, J = 6.23 Hz, 2H), 3.49 (t, J = 6.03 Hz, 2H), 2.55 (s, 3H); LCMS: ESI [M + H]+ = 511.1 |
| 317 | 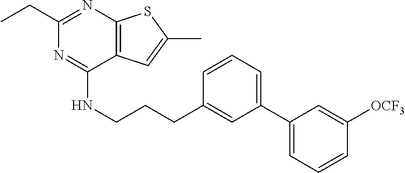 [SMILES: CCc1nc(NCCCc2cccc(-c3cccc(OC(F)(F)F)c3)c2)c2cc(C)sc2n1] | KH 4007 | Method A 3 h (80° C.) | 69% | Cs₂CO₃ 3 h (120° C.) Pressure vessel | 35% | 2-ethyl-6- methyl-N-(3-(3′- (trifluoromethoxy)- [1,1′-biphenyl]-3- yl)propyl)thieno [2,3- d]pyrimidin-4- amine | ¹H NMR (400 MHz, CHLOROFORM-d) δ 7.44-7.51 (m, 2H), 7.37-7.44 (m, 4H), 7.25 (s, 1H), 7.21 (d, J = 7.79 Hz, 1H), 6.63 (s, 1H), 3.71 (q, J = 6.75 Hz, 2H), 2.83 (q, J = 7.40 Hz, 4H), 2.46-2.54 (m, 3H), 2.09 (quin, J = 7.30 Hz, 2H), 1.34 (t, J = 7.59 Hz, 3H); LCMS: ESI [M + H]+ = 472.1 |
| 318 | 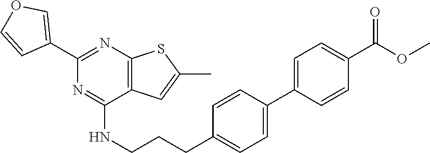 [SMILES: COC(=O)c1ccc(-c2ccc(CCCNc3nc(-c4ccoc4)nc4sc(C)cc34)cc2)cc1] | KH 6067 | Method A 1 d (90° C.) | 84% | Cs₂CO₃ 1 h (90° C.) | 11% | methyl 4′-(3- ((2-(furan-3- yl)-6- methylthieno[2, 3-d]pyrimidin-4- yl)amino)propyl)- [1,1′- biphenyl]-4- carboxylate | ¹H NMR (399 MHz, DMSO-d6) δ 8.17 (s, 1H), 7.99-8.07 (m, J = 8.17 Hz, 2H), 7.82 (d, J = 8.17 Hz, 3H), 7.64-7.71 (m, 3H), 7.35-7.41 (m, J = 8.17 Hz, 2H), 7.24 (s, 1H), 6.91 (s, 1H), 3.87 (s, 3H), 3.57 (t, J = 6.42 Hz, 2H), 3.31 (br. s., 1H), 2.75 (t, J = 7.40 Hz, 2H), 1.92-2.03 (m, 2H); |
| | | | | | | | | LCMS: ESI [M + H]+ = 484.1 |
| 319 | 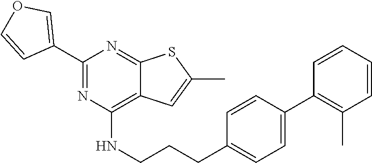 [SMILES: Cc1cc2c(NCCCc3ccc(-c4ccccc4C)cc3)nc(-c3ccoc3)nc2s1] | KH 6067 | Method A 1 d (60° C.) | 70% | Cs₂CO₃ 3 h (100° C.) | 18% | 2-(furan-3-yl)- 6-methyl-N-(3- (2′-methyl- [1,1′-biphenyl]-4- yl)propyl)thieno [2,3- d]pyrimidin-4- amine | ¹H NMR (399 MHz, METHANOL-d4) δ 8.11 (d, J = 0.78 Hz, 1H), 7.52 (t, J = 1.56 Hz, 1H), 7.26-7.31 (m, 2H), 7.13-7.26 (m, 6H), 7.05 (d, J = 1.17 Hz, 1H), 6.97-7.00 (m, 1H), 3.69 (t, J = 7.20 Hz, 2H), 2.80 (t, J = 7.20 Hz, 2H), 2.55 (d, J = 1.17 Hz, 3H), 2.22 (s, 3H), 2.09 (quin, J = 7.30 Hz, 1H); |
| | | | | | | | | LCMS: ESI [M + H]+ = 440.2 |
| 320 | 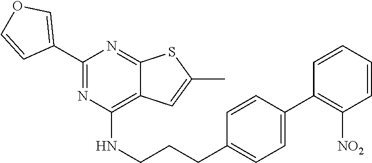 [SMILES: Cc1cc2c(NCCCc3ccc(-c4ccccc4[N+](=O)[O-])cc3)nc(-c3ccoc3)nc2s1] | KH 6067 | Method A 1 d (60° C.) | 70% | Cs₂CO₃ 1 d (100° C.) | 3% | 2-(furan-3-yl)- 6-methyl-N-(3- (2′-nitro-[1,1′- biphenyl]-4- yl)propyl)thieno [2,3- d]pyrimidin-4- amine | ¹H NMR (399 MHz, METHANOL-d4) δ 8.11 (s, 1H), 7.84 (d, J = 7.01 Hz, 1H), 7.65- 7.71 (m, J = 8.95 Hz, 1H), 7.51- 7.58 (m, 2H), 7.47 (d, J = 7.79 Hz, 1H), 7.28-7.35 (m, J = 8.17 Hz, 2H), 7.19-7.26 (m, 2H), 7.05 (s, 1H), 7.00 (s, 1H), 3.69 (t, J = 7.20 Hz, 2H), 2.81 (t, J = 7.59 Hz, 2H), 2.55 (s, 3H), 2.10 (td, J = |
| | | | | | | | | 7.49, 14.60 Hz, 2H); LCMS: |
| | | | | | | | | ESI [M + H]+ = 471.1 |
| 321 | 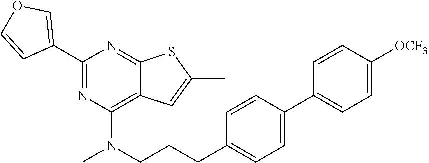 | KH 6067 | Method A 1 d (100° C.) | 48% | Cs₂CO₃ 2 h (100° C.) | 27% | 2-(furan-3-yl)- N,6-dimethyl- N-(3-(4′- (trifluoromethoxy)- [1,1′-biphenyl]-4- yl)propyl)thieno [2,3- d]pyrimidin-4- amine | ¹H NMR (399 MHz, CHLOROFORM-d) δ 8.07 (br. s., 1H), 7.59 (d, J = 8.56 Hz, 2H), 7.50 (d, J = 8.17 Hz, 2H), 7.42 (s, 1H), 7.29 (d, J = 8.17 Hz, 4H), 6.99 (br. s., 1H), 6.91 (br. s., 1H), 3.85 (t, J = 7.40 Hz, 2H), 3.35- 3.44 (m, 3H), 2.78 (t, J = 7.40 Hz, 2H), 2.48 (s, 3H), 2.12 (td, J = 7.30, 14.99 Hz, 2H); LCMS: ESI |
| | | | | | | | | [M + H]+ = 524.1 |
| 322 | 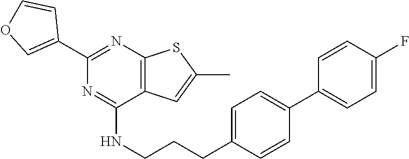 | KH 6067 | Method A 3 d (60° C.) 6 h (90° C.) | 89% | Cs₂CO₃ 2 h (80° C.) | 67% | N-(3-(4′-fluoro- [1,1′-biphenyl]- 4-yl)propyl)-2- (furan-3-yl)-6- methylthieno[2, 3-d]pyrimidin- 4-amine | ¹H NMR (399 MHz, ACETONITRILE-d3) δ 8.08 (s, 1H), 7.56-7.66 (m, 2H), 7.49 (d, J = 1.17 Hz, 3H), 7.29 (d, J = 7.79 Hz, 2H), 7.17 (t, J = 8.76 Hz, 2H), 6.92 (d, J = 14.01 Hz, 2H), 6.13 (br. s., 1H), 3.55-3.71 (m, 2H), 2.75 (t, J = 7.20 Hz, 2H), 2.50 (s, 3H), 2.00 (t, J = 7.01 Hz, 2H); LCMS: ESI [M + H]+ = |
| | | | | | | | | 444.0 |
| 323 | 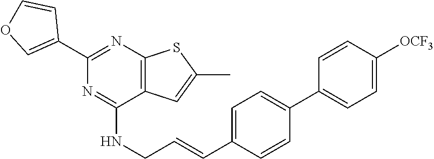 [SMILES: Cc1cc2c(NC/C=C/c3ccc(-c4ccc(OC(F)(F)F)cc4)cc3)nc(-c3ccoc3)nc2s1] | KH 6067 | Method A 3 d (60° C.) 6 h (90° C.) | 89% | Cs₂CO₃ 3 h (80° C.) | 49% | 2-(furan-3-yl)- 6-methyl-N- [(2E)-3-[4′- (trifluoromethoxy)- [1,1′- biphenyl]-4- yl]prop-2-en-1- yl]thieno[2,3- d]pyrimidin-4- amine | ¹H NMR (399 MHz, ACETONITRILE-d3) δ 8.22 (s, 1H), 7.71 (d, J = 8.95 Hz, 2H), 7.53-7.62 (m, 3H), 7.46-7.52 (m, 2H), 7.36 (d, J = 8.56 Hz, 2H), 7.02 (dd, J = 0.97, 13.04 Hz, 2H), 6.71 (d, J = 15.96 Hz, 1H), 6.50 (td, J = 6.13, 15.77 Hz, 1H), 6.38 (t, J = 5.45 Hz, 1H), 4.42 (t, J = 5.64 Hz, 2H), 2.55 (s, 3H); |
| | | | | | | | | LCMS: ESI [M + H]+ = 508.1 |
| 324 | 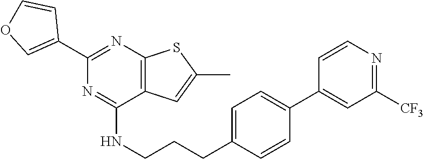 [SMILES: Cc1cc2c(NCCCc3ccc(-c4ccnc(C(F)(F)F)c4)cc3)nc(-c3ccoc3)nc2s1] | KH 6067 | Method A 3 d (60° C.) 6 h (90° C.) | 89% | Cs₂CO₃ 4 h (80° C.) | 49% | 2-(furan-3-yl)-6- methyl-N-(3- (4-[2- (trifluoromethyl) pyridin-4- yl]phenyl)propyl) thieno[2,3- d]pyrimidin-4- amine | ¹H NMR (399 MHz, ACETONITRILE-d3) δ 8.73 (d, J = 5.06 Hz, 1H), 8.06 (d, J = 0.78 Hz, 1H), 8.00 (s, 1H), 7.81 (d, J = 3.50 Hz, 1H), 7.65-7.71 (m, 2H), 7.49 (t, J = 1.75 Hz, 1H), 7.38 (d, J = 8.17 Hz, 2H), 6.92- 6.96 (m, 1H), 6.90 (d, J = 1.17 Hz, 1H), 6.14 (br. s., 1H), 3.64 (q, J = 6.75 Hz, 2H), 2.80 (t, J = 7.40 |
| | | | | | | | | Hz, 2H), 2.50 (d, J = 0.78 Hz, |
| | | | | | | | | 3H), 1.99-2.07 (m, 2H); LCMS: |
| | | | | | | | | ESI [M + H]+ = 495.0 |
| 325 | 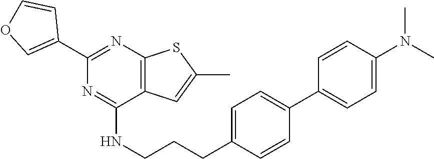 | KH 6067 | Method A 3 d (60° C.) 6 h (90° C.) | 89% | Cs₂CO₃ 4 h (80° C.) | 33% | N-(3-[4′- (dimethylamino)- [1,1′- biphenyl]-4- yl]propyl)-2- (furan-3-yl)-6- methylthieno[2, 3-d]pyrimidin- 4-amine | ¹H NMR (399 MHz, ACETONITRILE-d3) δ 8.10 (d, J = 1.56 Hz, 1H), 7.46-7.54 (m, 5H), 7.27 (d, J = 8.17 Hz, 2H), 6.93 (dd, J = 1.17, 7.01 Hz, 2H), 6.82 (d, J = 8.95 Hz, 2H), 6.14 (br. s., 1H), 3.64 (q, J = 6.75 Hz, 2H), 2.95 (s, 6H), 2.75 (t, J = 7.59 Hz, 2H), 2.52 (d, J = 1.17 Hz, 3H), 1.98-2.05 (m, 2H); LCMS: |
| | | | | | | | | ESI [M + H]+ = 469.2 |
| 326 | 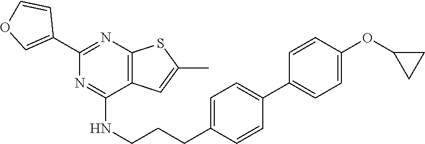 [SMILES: Cc1cc2c(NCCCc3ccc(-c4ccc(OC5CC5)cc4)cc3)nc(-c3ccoc3)nc2s1] | KH 6067 | Method A 3 d (60° C.) 6 h (90° C.) | 89% | Cs₂CO₃ 3 h (90° C.) | 46% | N-(3-(4′- cyclopropoxy- [1,1′-biphenyl]- 4-yl)propyl)-2- (furan-3-yl)-6- methylthieno[2, 3-d]pyrimidin- 4-amine | ¹H NMR (399 MHz, ACETONITRILE-d3) δ 8.09 (s, 1H), 7.47-7.59 (m, 5H), 7.29 (d, J = 8.17 Hz, 2H), 7.12 (d, J = 8.56 Hz, 2H), 6.90-6.97 (m, 2H), 6.14 (br. s., 1H), 3.75-3.85 (m, 1H), 3.64 (q, J = 6.62 Hz, 2H), 2.76 (t, J = 7.59 Hz, 2H), 2.51 (s, 3H), 2.01 (quin, J = 7.30 |
| | | | | | | | | Hz, 2H), 0.76-0.85 (m, 2H), |
| | | | | | | | | 0.69 (br. s., 2H); LCMS: ESI |
| | | | | | | | | [M + H]+ = 482.1 |
| 327 | 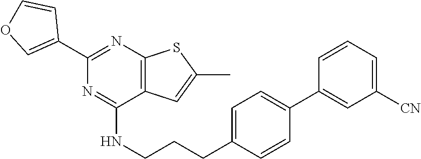 [SMILES: Cc1cc2c(NCCCc3ccc(-c4cccc(C#N)c4)cc3)nc(-c3ccoc3)nc2s1] | KH 6067 | Method A 3 d (60° C.) 6 h (90° C.) | 89% | Cs₂CO₃ 1.5 h (80° C.) | 52% | 4′-(3-([2- (furan-3-yl)-6- methylthieno[2, 3-d]pyrimidin- 4-yl]amino) propyl)- [1,1′- biphenyl]-3- carbonitrile | ¹H NMR (399 MHz, ACETONITRILE-d3) δ 8.08 (s, 1H), 7.96 (s, 1H), 7.89 (d, J = 7.79 Hz, 1H), 7.68 (d, J = 7.40 Hz, 1H), 7.57-7.62 (m, 1H), 7.55 (d, J = 8.17 Hz, 2H), 7.50 (s, 1H), 7.34 (d, J = 7.79 Hz, 2H), 6.93 (d, J = 16.74 Hz, 2H), 6.14 (br. s., 1H), 3.64 (q, J = 6.62 Hz, 2H), 2.78 (t, J = 7.40 Hz, 2H), |
| | | | | | | | | 2.51 (s, 3H), 2.02 (quin, J = 7.30 |
| | | | | | | | | Hz, 2H); LCMS: ESI [M + H]+ = |
| | | | | | | | | 451.0 |
| 328 | 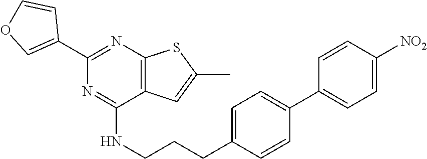 | KH 6067 | Method A 3 d (60° C.) 6 h (90° C.) | 89% | Cs₂CO₃ 2 h (80° C.) | 25% | 2-(furan-3-yl)- 6-methyl-N-(3- (4′-nitro-[1,1′- biphenyl]-4- yl)propyl)thieno [2,3- d]pyrimidin-4- amine | ¹H NMR (399 MHz, DMSO-d6) δ 8.27 (d, J = 8.17 Hz, 2H), 8.13 (s, 1H), 7.92 (d, J = 8.56 Hz, 2H), 7.78 (t, J = 5.45 Hz, 1H), 7.70 (d, J = 8.17 Hz, 2H), 7.65 (s, 1H), 7.38 (d, J = 7.79 Hz, 2H), 7.21 (s, 1H), 6.87 (s, 1H), 3.48-3.62 (m, 2H), 2.74 (t, J = 7.40 Hz, 2H), 2.50 (s, 3H), 1.95 (t, J = 7.20 Hz, 2H); LCMS: ESI [M + H]+ = |
| | | | | | | | | 471.1 |
| 329 | 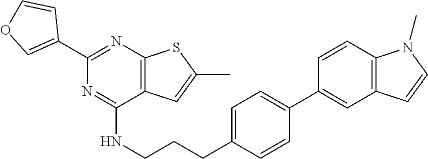 [SMILES: Cc1cc2c(NCCCc3ccc(-c4ccc5c(ccn5C)c4)cc3)nc(-c3ccoc3)nc2s1] | KH 6067 | Method A 3 d (60° C.) 6 h (90° C.) | 89% | Cs₂CO₃ 2 h (80° C.) | 17% | 2-(furan-3-yl)- 6-methyl-N-(3- [4-(1-methyl- 1H-indol-5- yl)phenyl]propyl) thieno[2,3- d]pyrimidin- 4-amine | ¹H NMR (399 MHz, ACETONITRILE-d3) δ 8.11 (s, 1H), 7.81 (s, 1H), 7.60 (d, J = 8.17 Hz, 2H), 7.42-7.52 (m, 3H), 7.32 (d, J = 8.17 Hz, 2H), 7.20 (d, J = 3.11 Hz, 1H), 6.91- 6.97 (m, 2H), 6.49 (d, J = 3.11 Hz, 1H), 6.17 (br. s., 1H), 3.82 (s, 3H), 3.67 (q, J = 7.01 Hz, 2H), 2.79 (t, J = 7.20 Hz, 2H), 2.52 (s, |
| | | | | | | | | 3H), 2.03-2.05 (m, 2H); LCMS: |
| | | | | | | | | ESI [M + H]+ = 479.2 |
| 330 | 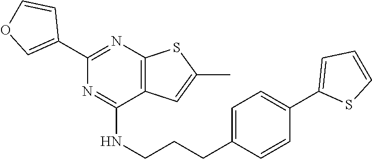 | KH 6067 | Method A 3 d (60° C.) 6 h (90° C.) | 89% | Cs₂CO₃ 1.5 h (80° C.) | 79% | 2-(furan-3-yl)- 6-methyl-N-(3- [4-(thiophen-2- yl)phenyl]propyl) thieno[2,3- d]pyrimidin-4- amine | ¹H NMR (399 MHz, ACETONITRILE-d3) δ 8.11 (s, 1H), 7.55 (d, J = 8.17 Hz, 2H), 7.50 (s, 1H), 7.35 (d, J = 4.28 Hz, 2H), 7.25 (d, J = 8.17 Hz, 2H), 7.09 (t, J = 4.28 Hz, 1H), 6.92 (d, J = 16.35 Hz, 2H), 6.12 (br. s., 1H), 3.62 (q, J = 6.49 Hz, 2H), 2.73 (t, J = 7.40 Hz, 2H), 2.50 (s, 3H), 1.98-2.04 (m, 2H); LCMS: |
| | | | | | | | | ESI [M + H]+ = 432.0 |
| 331 | 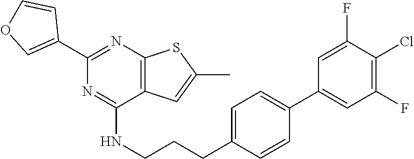 [SMILES: Cc1cc2c(NCCCc3ccc(-c4cc(F)c(Cl)c(F)c4)cc3)nc(-c3ccoc3)nc2s1] | KH 6067 | Method A 3 d (60° C.) 6 h (90° C.) | 89% | Cs₂CO₃ 1.5 h (80° C.) | 63% | N-(3-(4′- chloro-3′,5′- difluoro-[1,1′- biphenyl]-4- yl)propyl)-2- (furan-3-yl)-6- methylthieno[2, 3-d]pyrimidin- 4-amine | ¹H NMR (399 MHz, ACETONITRILE-d3) δ 8.07 (s, 1H), 7.47-7.56 (m, 3H), 7.34 (dd, J = 8.17, 16.74 Hz, 4H), 6.92 (d, J = 14.40 Hz, 2H), 6.13 (br. s., 1H), 3.63 (q, J = 6.49 Hz, 2H), 2.77 (t, J = 7.40 Hz, 2H), 2.51 (s, 3H), 2.01 (t, J = 7.40 Hz, 2H); LCMS: ESI [M + H]+ = 496.1 |
| 332 | 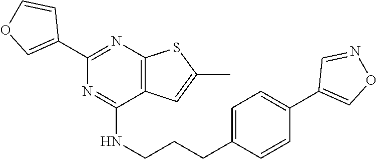 [SMILES: Cc1cc2c(NCCCc3ccc(-c4cnoc4)cc3)nc(-c3ccoc3)nc2s1] | KH 6067 | Method A 3 d (60° C.) 6 h (90° C.) | 89% | Cs₂CO₃ 2.5 h (80° C.) 0.5 h (95° C.) | 6% | 2-(furan-3-yl)- 6-methyl-N-(3- [4-(1,2-oxazol- 4-yl)phenyl] propyl) thieno[2,3- d]pyrimidin-4- amine | ¹H NMR (399 MHz, METHANOL-d4) δ 8.99 (s, 1H), 8.77 (s, 1H), 8.01 (s, 1H), 7.49 (d, J = 6.62 Hz, 3H), 7.26 (d, J = 7.79 Hz, 2H), 6.99 (s, 1H), 6.93 (s, 1H), 3.65 (t, J = 7.20 Hz, 2H), 2.75 (t, J = 7.40 Hz, 2H), 2.51 (s, 3H), 2.04 (t, J = 7.20 Hz, 2H); LCMS: ESI [M + H]+ = 417.1 |
| 333 | 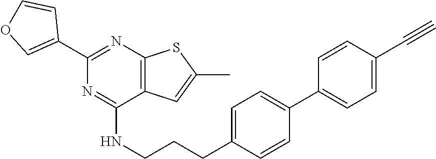 | KH 6067 | Method A 3 d (60° C.) 6 h (90° C.) | 89% | Cs₂CO₃ 1.5 h (80° C.) | 30% | N-(3-(4′- ethynyl-[1,1′- biphenyl]-4- yl)propyl)-2- (furan-3-yl)-6- methylthieno[2, 3-d]pyrimidin- 4-amine | ¹H NMR (399 MHz, ACETONITRILE-d3) δ 8.09 (s, 1H), 7.60-7.67 (m, 2H), 7.57 (d, J = 6.62 Hz, 4H), 7.50 (s, 1H), 7.34 (d, J = 7.79 Hz, 2H), 6.93 (d, J = 10.12 Hz, 2H), 6.16 (br. s., 1H), 3.65 (q, J = 6.62 Hz, 2H), 3.45 (s, 1H), 2.79 (t, J = 7.40 Hz, 2H), 2.52 (s, 3H), 2.00-2.08 (m, 2H); LCMS: ESI [M + H]+ = |
| | | | | | | | | 450.0 |
| 334 | 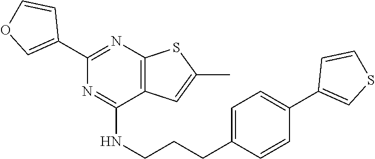 [SMILES: Cc1cc2c(NCCCc3ccc(-c4ccsc4)cc3)nc(-c3ccoc3)nc2s1] | KH 6067 | Method A 3 d (60° C.) 6 h (90° C.) | 89% | Cs₂CO₃ 1.5 h (80° C.) | 68% | 2-(furan-3-yl)- 6-methyl-N-(3- [4-(thiophen-3- yl)phenyl]propyl) thieno[2,3- d]pyrimidin-4- amine | ¹H NMR (399 MHz, ACETONITRILE-d3) δ 8.10 (s, 1H), 7.54-7.65 (m, 3H), 7.41- 7.51 (m, 3H), 7.29 (d, J = 7.79 Hz, 2H), 6.93 (d, J = 9.34 Hz, 2H), 6.16 (br. s., 1H), 3.65 (q, J = 6.62 Hz, 2H), 2.76 (t, J = 7.40 Hz, 2H), 2.52 (s, 3H), 2.03 (d, J = 7.01 Hz, 2H); LCMS: ESI [M + H]+ = 432.1 |
| 335 | 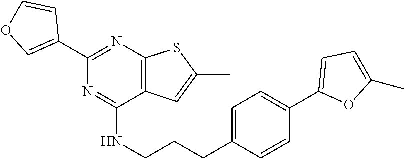 [SMILES: Cc1ccc(-c2ccc(CCCNc3nc(-c4ccoc4)nc4sc(C)cc34)cc2)o1] | KH 6067 | Method A 3 d (60° C.) 6 h (90° C.) | 89% | Cs₂CO₃ 3 h (90° C.) | 61% | 2-(furan-3-yl)- 6-methyl-N-(3- [4-(5- methylfuran-2- yl)phenyl]propyl) thieno[2,3- d]pyrimidin-4- amine | ¹H NMR (399 MHz, ACETONITRILE-d3) δ 8.10 (s, 1H), 7.45-7.57 (m, 3H), 7.24 (d, J = 7.79 Hz, 2H), 6.91 (d, J = 13.62 Hz, 2H), 6.58 (br. s., 1H), 6.10 (br. s., 2H), 3.62 (q, J = 6.36 Hz, 2H), 2.72 (t, J = 7.20 Hz, 2H), 2.50 (s, 3H), 2.33 (s, 3H), 1.97-2.05 (m, 2H); LCMS: ESI [M + H]+ = 430.0 |
| 336 | 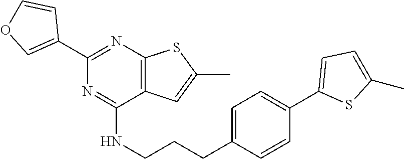 | KH 6067 | Method A 3 d (60° C.) 6 h (90° C.) | 89% | Cs₂CO₃ 2 h (90° C.) | 73% | 2-(furan-3-yl)- 6-methyl-N-(3- [4-(5- methylthiophen- 2-yl)phenyl] propyl)thieno[2,3- d]pyrimidin-4- amine | ¹H NMR (399 MHz, ACETONITRILE-d3) δ 8.09 (s, 1H), 7.42-7.53 (m, 3H), 7.21 (d, J = 8.17 Hz, 2H), 7.12 (d, J = 3.50 Hz, 1H), 6.91 (d, J = 14.40 Hz, 2H), 6.74 (d, J = 2.34 Hz, 1H), 6.14 (br. s., 1H), 3.60 (q, J = 6.62 Hz, 2H), 2.71 (t, J = 7.40 Hz, 2H), 2.48 (d, J = 12.07 Hz, 6H), 1.96-2.03 (m, 2H); LCMS: |
| | | | | | | | | ESI [M + H]+ = 446.1 |
| 337 | 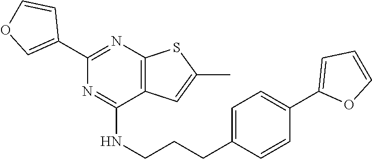 | KH 6067 | Method A 3 d (60° C.) 6 h (90° C.) | 89% | Cs₂CO₃ 2 h (90° C.) | 85% | N-(3-[4-(furan- 2-yl)phenyl] propyl)-2-(furan- 3-yl)-6- methylthieno[2, 3-d]pyrimidin- 4-amine | ¹H NMR (399 MHz, ACETONITRILE-d3) δ 8.09 (s, 1H), 7.59 (d, J = 7.40 Hz, 2H), 7.53 (s, 1H), 7.49 (s, 1H), 7.26 (d, J = 7.40 Hz, 2H), 6.91 (d, J = 12.85 Hz, 2H), 6.71 (br. s., 1H), 6.51 (br. s., 1H), 6.13 (br. s., 1H), 3.62 (q, J = 6.10 Hz, 2H), 2.73 (t, J = 7.40 Hz, 2H), 2.50 (s, 3H), 1.99 (d, J = 7.40 Hz, 2H); LCMS: |
| | | | | | | | | ESI [M + H]+ = 416.1 |
| 338 | 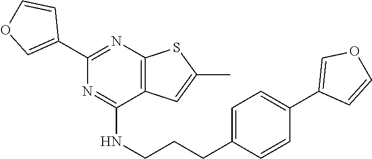 [SMILES: Cc1cc2c(NCCCc3ccc(-c4ccoc4)cc3)nc(-c3ccoc3)nc2s1] | KH 6067 | Method A 3 d (60° C.) 6 h (90° C.) | 89% | Cs₂CO₃ 2 h (90° C.) | 47% | 2-(furan-3-yl)- N-(3-[4-(furan- 3-yl)phenyl] propyl)-6- methylthieno[2, 3-d]pyrimidin- 4-amine | ¹H NMR (399 MHz, ACETONITRILE-d3) δ 8.09 (s, 1H), 7.85 (s, 1H), 7.55 (s, 1H), 7.39-7.51 (m, 3H), 7.25 (d, J = 7.01 Hz, 2H), 6.92 (d, J = 9.34 Hz, 2H), 6.79 (br. s., 1H), 6.15 (br. s., 1H), 3.62 (d, J = 5.84 Hz, 2H), 2.68-2.78 (m, 2H), 2.51 (br. s., 3H), 1.99 (br. s., 2H); LCMS: ESI [M + H]+ = 416.1 |
| 339 | 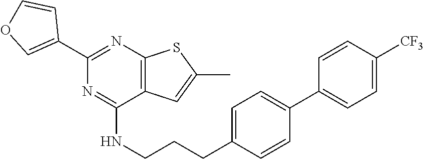 [SMILES: Cc1cc2c(NCCCc3ccc(-c4ccc(C(F)(F)F)cc4)cc3)nc(-c3ccoc3)nc2s1] | KH 6067 | Method A 3 d (60° C.) 6 h (90° C.) | 89% | Cs₂CO₃ 2.5 h (80° C.) | 62% | 2-(furan-3-yl)- 6-methyl-N-(3-[4′- (trifluoromethyl)- [1,1′-biphenyl]-4- yl]propyl)thieno [2,3- d]pyrimidin-4- amine | ¹H NMR (399 MHz, ACETONITRILE-d3) δ 8.09 (s, 1H), 7.72-7.84 (m, 4H), 7.61 (d, J = 8.17 Hz, 2H), 7.51 (s, 1H), 7.37 (d, J = 7.79 Hz, 2H), 6.94 (d, J = 9.73 Hz, 2H), 6.16 (br. s., 1H), 3.66 (q, J = 6.62 Hz, 2H), 2.80 (t, J = 7.40 Hz, 2H), 2.52 (s, 3H), 2.01-2.10 (m, 2H); LCMS: ESI [M + H]+ = 494.1 |
| 340 | 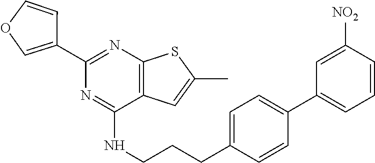 [SMILES: Cc1cc2c(NCCCc3ccc(-c4cccc([N+](=O)[O-])c4)cc3)nc(-c3ccoc3)nc2s1] | KH 6067 | Method A 3 d (60° C.) 6 h (90° C.) | 89% | Cs₂CO₃ 2 h (90° C.) | 48% | 2-(furan-3-yl)-6- methyl-N-(3- (3′-nitro-[1,1′- biphenyl]-4- yl)propyl)thieno [2,3- d]pyrimidin-4- amine | ¹H NMR (399 MHz, CHLOROFORM-d) δ 8.43 (s, 1H), 8.17 (d, J = 8.17 Hz, 1H), 8.10 (s, 1H), 7.88 (d, J = 7.79 Hz, 1H), 7.52-7.65 (m, 3H), 7.42 (s, 1H), 7.32 (d, J = 7.79 Hz, 2H), 7.01 (s, 1H), 6.60 (s, 1H), 4.92- 5.00 (m, 1H), 3.72 (q, J = 6.23 Hz, 2H), 2.81 (t, J = 7.40 Hz, 2H), 2.47 (s, 3H), 1.99-2.16 (m, |
| | | | | | | | | 2H); LCMS: ESI [M + H]+ = |
| | | | | | | | | 471.1 |
| 341 | 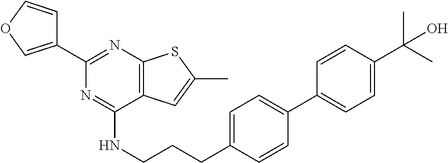 [SMILES: Cc1cc2c(NCCCc3ccc(-c4ccc(C(C)(C)O)cc4)cc3)nc(-c3ccoc3)nc2s1] | KH 6067 | Method A 3 d (60° C.) 6 h (90° C.) | 89% | Cs₂CO₃ 2 h (80° C.) | 34% | 2-[4′-(3-([2- (furan-3-yl)-6- methylthieno[2, 3-d]pyrimidin- 4-yl]amino) propy1)-[1,1′- biphenyl]-4- yl]propan-2-ol | ¹H NMR (399 MHz, ACETONITRILE-d3) δ 8.10 (s, 1H), 7.45-7.62 (m, 7H), 7.32 (d, J = 7.79 Hz, 2H), 6.93 (d, J = 8.95 Hz, 2H), 6.15 (br. s., 1H), 3.65 (q, J = 6.62 Hz, 2H), 3.15 (s, 1H), 2.78 (t, J = 7.40 Hz, 2H), 2.51 (s, 3H), 2.00-2.05 (m, 2H), 1.51 (s, 6H); LCMS: ESI [M + H]+ = 484.2 |
| 342 | 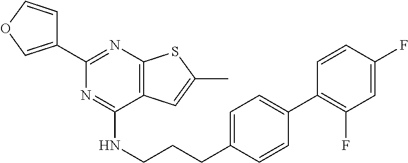 [SMILES: Cc1cc2c(NCCCc3ccc(-c4ccc(F)cc4F)cc3)nc(-c3ccoc3)nc2s1] | KH 6067 | Method A 3 d (60° C.) 6 h (90° C.) | 89% | Cs₂CO₃ 2 h (80° C.) | 68% | N-(3-(2′,4′- difluoro-[1,1′- biphenyl]-4- yl)propyl)-2- (furan-3-yl)-6- methylthieno[2, 3-d]pyrimidin- 4-amine | ¹H NMR (399 MHz, CHLOROFORM-d) δ 8.15 (s, 1H), 7.38-7.53 (m, 4H), 7.23- 7.35 (m, 2H), 7.04 (s, 1H), 6.87- 6.99 (m, 2H), 6.60 (s, 1H), 4.97 (br. s., 1H), 3.74 (q, J = 6.62 Hz, 2H), 2.82 (t, J = 7.20 Hz, 2H), 2.50 (s, 3H), 2.10 (t, J = 7.01 Hz, 2H); LCMS: ESI [M + H]+ = 462.0 |
| 343 | 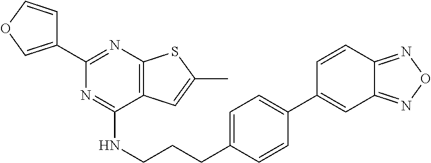 | KH 6067 | Method A 3 d (60° C.) 6 h (90° C.) | 89% | Cs₂CO₃ 2.5 h (90° C.) | 58% | N-(3-[4-(2,1,3- benzoxadiazol- 5-yl)phenyl] propyl)-2-(furan- 3-yl)-6- methylthieno[2, 3-d]pyrimidin- 4-amine | ¹H NMR (399 MHz, CHLOROFORM-d) δ 8.12 (s, 1H), 7.86-7.96 (m, 2H), 7.71 (d, J = 9.34 Hz, 1H), 7.59 (d, J = 7.79 Hz, 2H), 7.45 (s, 1H), 7.37 (d, J = 7.79 Hz, 2H), 7.04 (s, 1H), 6.64 (s, 1H), 5.00 (br. s., 1H), 3.75 (q, J = 6.36 Hz, 2H), 2.85 (t, J = 7.40 Hz, 2H), 2.51 (s, 3H), 2.06-2.18 (m, 2H); LCMS: ESI |
| | | | | | | | | [M + H]+ = 468.1 |
| 344 | 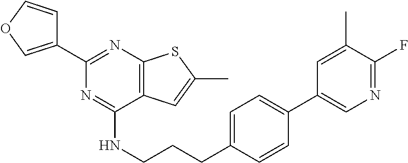 | KH 6067 | Method A 3 d (60° C.) 6 h (90° C.) | 89% | Cs₂CO₃ 2.5 h (85° C.) | 64% | N-(3-[4-(6- fluoro-5- methylpyridin- 3-yl)phenyl] propyl)-2-(furan- 3-yl)-6- methylthieno[2, 3-d]pyrimidin- 4-amine | ¹H NMR (399 MHz, CHLOROFORM-d) δ 8.22 (s, 1H), 8.12 (s, 1H), 7.76 (d, J = 9.34 Hz, 1H), 7.42-7.50 (m, 3H), 7.32 (d, J = 7.79 Hz, 2H), 7.04 (s, 1H), 6.61 (s, 1H), 4.98 (br. s., 1H), 3.73 (q, J = 6.62 Hz, 2H), 2.82 (t, J = 7.20 Hz, 2H), 2.50 (s, 3H), 2.36 (s, 3H), 2.09 (quin, J = 7.10 Hz, 2H); LCMS: |
| | | | | | | | | ESI [M + H]+ = 459.1 |
| 345 | 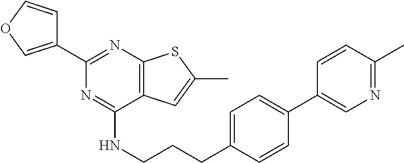 [SMILES: Cc1ccc(-c2ccc(CCCNc3nc(-c4ccoc4)nc4sc(C)cc34)cc2)cn1] | KH 6067 | Method A 3d (60° C.) 6h (90° C.) | 89% | Cs₂CO₃ 1.5 h (85° C.) | 25% | 2-(furan-3-yl)- 6-methyl-N-(3- [4-(6- methylpyridin- 3-yl)phenyl] propyl)thieno [2,3- d]pyrimidin-4- amine | ¹H NMR (399 MHz, ACETONITRILE-d3) δ 8.69 (s, 1H), 8.08 (s, 1H), 7.83 (d, J = 7.79 Hz, 1H), 7.46-7.58 (m, 3H), 7.34 (d, J = 8.17 Hz, 2H), 7.26 (d, J = 8.17 Hz, 1H), 6.93 (d, J = 10.12 Hz, 2H), 6.18 (br. s., 1H), 3.64 (q, J = 6.62 Hz, 2H), 2.78 (t, J = 7.40 Hz, 2H), 2.52 (d, J = 5.06 Hz, 6H), 2.00-2.08 (m, |
| | | | | | | | | 2H); LCMS: ESI [M + H]+ = |
| | | | | | | | | 441.1 |
| 346 | 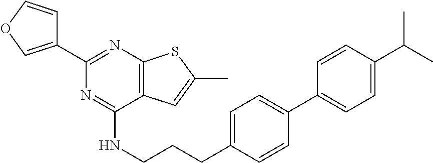 | KH 6067 | Method A 3 d (60° C.) 6 h (90° C.) | 89% | Cs₂CO₃ 1.5 h (85° C.) | 53% | 2-(furan-3-yl)- 6-methyl-N-(3- [4′-(propan-2- yl)-[1,1′- biphenyl]-4- yl]propyl)thieno [2,3- d]pyrimidin-4- amine | ¹H NMR (399 MHz, ACETONITRILE-d3) δ 8.09 (s, 1H), 7.46-7.58 (m, 5H), 7.32 (dd, J = 4.87, 7.98 Hz, 4H), 6.93 (d, J = 8.18 Hz, 2H), 6.17 (br. s., 1H), 3.65 (q, J = 6.88 Hz, 2H), 2.95 (td, J = 6.96, 13.72 Hz, 1H), 2.77 (t, J = 7.40 Hz, 2H), 2.51 (s, 3H), 2.00-2.06 (m, 2H), 1.26 (d, J = 7.01 Hz, 6H); LCMS: ESI |
| | | | | | | | | [M + H]+ = 468.2 |
| 347 | 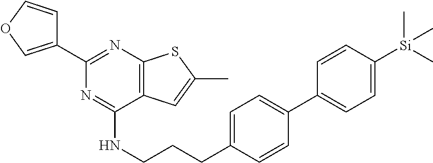 [SMILES: Cc1cc2c(NCCCc3ccc(-c4ccc([Si](C)(C)C)cc4)cc3)nc(-c3ccoc3)nc2s1] | LM 6067 | Method A 3 d (60° C.) 6 h (90° C.) | 89% | Cs₂CO₃ 1.5 h (80° C.) | 54% | 2-(furan-3-yl)- 6-methyl-N-(3-[4′- (trimethylsilyl)- [1,1′-biphenyl]-4- yl]propyl)thieno [2,3- d]pyrimidin-4- amine | ¹H NMR (399 MHz, ACETONITRILE-d3) δ 8.09 (s, 1H), 7.47-7.64 (m, 7H), 7.26- 7.33 (m, 2H), 6.93 (d, J = 13.62 Hz, 2H), 6.10-6.20 (m, 1H), 3.63 (q, J = 6.62 Hz, 2H), 2.76 (t, J = 7.20 Hz, 2H), 2.50 (s, 3H), 2.01 (t, J = 7.40 Hz, 2H), 0.27 (s, 9H); LCMS: ESI [M + H]+ = 498.1 |
| 348 | 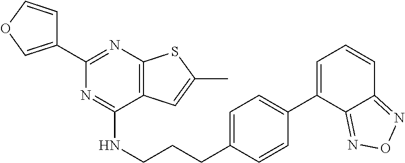 [SMILES: Cc1cc2c(NCCCc3ccc(-c4cccc5nonc45)cc3)nc(-c3ccoc3)nc2s1] | LM 6067 | Method A 3 d (60° C.) 6 h (90° C.) | 89% | K₂CO₃ 2 h (85° C.) | 60% | N-(3-[4-(2,1,3- benzoxadiazol- 4-yl)phenyl] propyl)-2-(furan- 3-yl)-6- methylthieno[2, 3-d]pyrimidin- 4-amine | ¹H NMR (399 MHz, ACETONITRILE-d3) δ 8.11 (s, 1H), 7.94 (d, J = 8.17 Hz, 2H), 7.85 (d, J = 8.95 Hz, 1H), 7.69 (d, J = 6.62 Hz, 1H), 7.56-7.64 (m, 1H), 7.50 (s, 1H), 7.43 (d, J = 8.17 Hz, 2H), 6.90-6.99 (m, 2H), 6.18 (br. s., 1H), 3.67 (q, J = 6.62 Hz, 2H), 2.83 (t, J = 7.59 Hz, 2H), 2.51 (s, 3H), 2.03-2.10 |
| | | | | | | | | (m, 2H); LCMS: ESI [M + H]+ = |
| | | | | | | | | 468.1 |
| 349 | 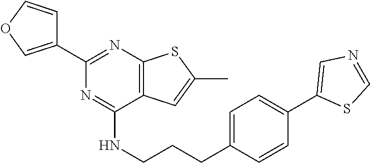 [SMILES: Cc1cc2c(NCCCc3ccc(-c4cncs4)cc3)nc(-c3ccoc3)nc2s1] | LM 6067 | Method A 3 d (60° C.) 6 h (90° C.) | 89% | K₂CO₃ 1 d (100° C.) | 6% | 2-(furan-3-yl)- 6-methyl-N-(3- [4-(1,3-thiazol- 5-yl)phenyl] propyl)thieno [2,3-d]pyrimidin- 4-amine | ¹H NMR (399 MHz, ACETONITRILE-d3) δ 8.80 (s, 1H), 8.09 (s, 2H), 7.56 (d, J = 7.79 Hz, 2H), 7.50 (s, 1H), 7.31 (d, J = 7.79 Hz, 2H), 6.92 (d, J = 9.73 Hz, 2H), 6.15 (br. s., 1H), 3.64 (q, J = 6.36 Hz, 2H), 2.77 (t, J = 7.40 Hz, 2H), 2.51 (s, 3H), 2.03 (d, J = 7.01 Hz, 2H); LCMS: ESI [M + H]+ = 433.1 |
| 350 | 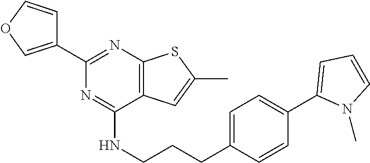 [SMILES: Cc1cc2c(NCCCc3ccc(-c4cccn4C)cc3)nc(-c3ccoc3)nc2s1] | LM 6067 | Method A 3 d (60° C.) 6 h (90° C.) | 89% | K₂CO₃ ₂ h (100° C.) | 54% | 2-(furan-3-yl)- 6-methyl-N-(3- [4-(1-methyl- 1H-pyrrol-2- yl)phenyl] propyl)thieno [2,3-d] pyrimidin-4- amine | ¹H NMR (399 MHz, ACETONITRILE-d3) δ 8.10 (s, 1H), 7.51 (s, 1H), 7.23-7.36 (m, 4H), 6.94 (d, J = 10.51 Hz, 2H), 6.71 (s, 1H), 6.03-6.22 (m, 3H), 3.52-3.71 (m, 5H), 2.75 (t, J = 7.59 Hz, 2H), 2.52 (s, 3H), 2.01 (t, J = 7.20 Hz, 2H); LCMS: ESI [M + H]+ = 429.0 |
| 351 | 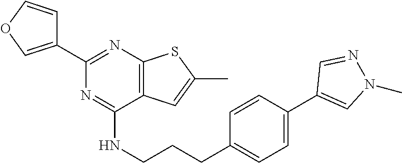 [SMILES: Cc1cc2c(NCCCc3ccc(-c4cnn(C)c4)cc3)nc(-c3ccoc3)nc2s1] | LM 6067 | Method A 3 d (60° C.) 6 h (90° C.) | 89% | K₂CO₃ 2 h (85° C.) | 60% | 2-(furan-3-yl)- 6-methyl-N-(3- [4-(1-methyl- 1H-pyrazol-4- yl)phenyl] propyl)thieno [2,3-d]pyrimidin- 4-amine | ¹H NMR (399 MHz, CHLOROFORM-d) δ 8.14 (s, 1H), 7.77 (s, 1H), 7.60 (s, 1H), 7.38-7.47 (m, 3H), 7.23 (d, J = 7.79 Hz, 2H), 7.03 (s, 1H), 6.53 (s, 1H), 4.91 (br. s., 1H), 3.95 (s, 3H), 3.72 (q, J = 6.49 Hz, 2H), 2.78 (t, J = 7.20 Hz, 2H), 2.46 (s, 3H), 2.01-2.14 (m, 2H); LCMS: ESI [M + H]+ = 430.1 |
| 352 | 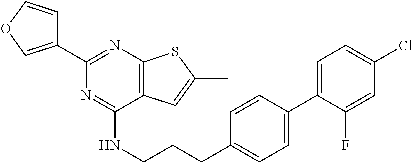 | LM 6067 | Method A 3 d (60° C.) 6 h (90° C.) | 89% | K₂CO₃ 1 h (100° C.) | 73% | N-(3-(4′- chloro-2′- fluoro-[1,1′- biphenyl]-4- yl)propyl)-2- (furan-3-yl)-6- methylthieno[2, 3-d]pyrimidin- 4-amine | ¹H NMR (399 MHz, CHLOROFORM-d) δ 8.15 (s, 1H), 7.43-7.53 (m, 3H), 7.34- 7.41 (m, 1H), 7.31 (d, J = 7.79 Hz, 2H), 7.17-7.24 (m, 2H), 7.04 (s, 1H), 6.61 (s, 1H), 4.97 (t, J = 5.06 Hz, 1H), 3.74 (q, J = 6.49 Hz, 2H), 2.82 (t, J = 7.20 Hz, 2H), 2.51 (s, 3H), 2.10 (quin, J = 7.10 Hz, 2H); LCMS: ESI |
| | | | | | | | | [M + H]+ = 478.0 |
| 353 | 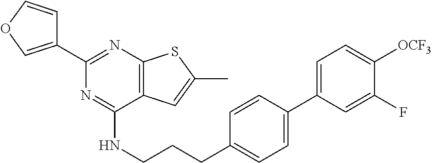 [SMILES: Cc1cc2c(NCCCc3ccc(-c4ccc(OC(F)(F)F)c(F)c4)cc3)nc(-c3ccoc3)nc2s1] | LM 6067 | Method A 3 d (60° C.) 6 h (90° C.) | 89% | K₂CO₃ 2 h (80° C.) | 64% | N-(3-[3′-fluoro-4′- (trifluoromethoxy)- [1,1′-biphenyl]-4- yl]propyl)-2- (furan-3-yl)-6- methylthieno [2,3-d]pyrimidin- 4-amine | ¹H NMR (399 MHz, CHLOROFORM-d) δ 8.13 (s, 1H), 7.28-7.53 (m, 8H), 7.04 (s, 1H), 6.59 (s, 1H), 4.95 (br. s., 1H), 3.74 (q, J = 6.23 Hz, 2H), 2.82 (t, J = 7.20 Hz, 2H), 2.49 (s, 3H), 2.02-2.17 (m, 2H); LCMS: ESI [M + H]+ = 528.1 |
| 354 | 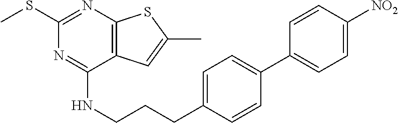 [SMILES: CSc1nc(NCCCc2ccc(-c3ccc([N+](=O)[O-])cc3)cc2)c2cc(C)sc2n1] | KH 5002 | Method A 2 h (60° C.) | 89% | Cs₂CO₃ 2 h (90° C.) | 30% | 6-methyl-2- (methylsulfanyl)- N-(3-(4′-nitro- [1,1′-biphenyl]-4- yl)propyl)thieno [2,3- d]pyrimidin-4- amine | ¹H NMR (399 MHz, CHLOROFORM-d) δ 8.29 (d, J = 8.56 Hz, 2H), 7.71 (d, J = 8.56 Hz, 2H), 7.56 (d, J = 8.18 Hz, 2H), 7.33 (d, J = 8.18 Hz, 2H), 6.53 (s, 1H), 4.97 (br. s., 1H), 3.67 (q, J = 6.36 Hz, 2H), 2.80 (t, J = 7.40 Hz, 2H), 2.54 (s, 3H), |
| | | | | | | | | 2.44 (s, 3H), 2.00-2.13 (m, 2H); |
| | | | | | | | | LCMS: ESI [M + H]+ = 451.1 |
| 355 | 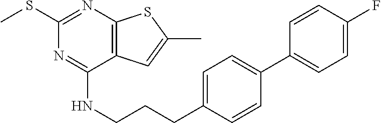 [SMILES: CSc1nc(NCCCc2ccc(-c3ccc(F)cc3)cc2)c2cc(C)sc2n1] | KH 5002 | Method A 2 h (60° C.) | 89% | Cs₂CO₃ 2 h (90° C.) | 42% | N-(3-(4′-fluoro- [1,1′-biphenyl]- 4-yl)propyl)-6- methyl-2- (methylsulfanyl) thieno[2,3- d]pyrimidin-4- amine | ¹H NMR (399 MHz, CHLOROFORM-d) δ 7.45-7.57 (m, 4H), 7.25-7.30 (m, 2H), 7.12 (t, J = 8.56 Hz, 2H), 6.45 (s, 1H), 4.89 (br. s., 1H), 3.67 (q, J = 6.62 Hz, 2H), 2.79 (t, J = 7.40 Hz, 2H), 2.54 (s, 3H), 2.41 (s, 3H), 2.05 (quin, J = 7.01 Hz, 2H); LCMS: ESI [M + H]+ = 424.1 |
| 356 | 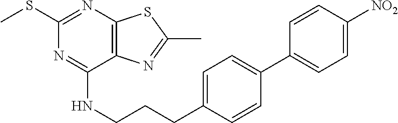 [SMILES: CSc1nc(NCCCc2ccc(-c3ccc([N+](=O)[O-])cc3)cc2)c2nc(C)sc2n1] | LM 13029 | Method A 2 h (60° C.) | 99% | Cs₂CO₃ 2 h (90° C.) | 48% | 2-methyl-5- (methylsulfanyl)- N-(3-(4′- nitro-[1,1′- biphenyl]-4- yl)propyl)- [1,3]thiazolo[5, 4-d]pyrimidin- | ¹H NMR (399 MHz, CHLOROFORM-d) δ 8.30 (d, J = 8.95 Hz, 2H), 7.73 (d, J = 8.56 Hz, 2H), 7.57 (d, J = 8.17 Hz, 2H), 7.34 (d, J = 8.18 Hz, 2H), 5.96 (br. s., 1H), 3.69 (q, J = 6.36 Hz, 2H), 2.82 (t, J = 7.59 Hz, 2H), 2.72 (s, 3H), 2.54 (s, 3H), |
| | | | | | | | 7-amine | 2.07 (quin, J = 7.20 Hz, 2H); |
| | | | | | | | | LCMS: ESI [M + H]+ = 452.1 |
| 357 | 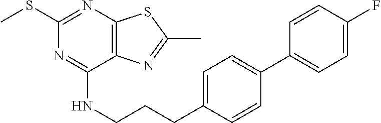 [SMILES: CSc1nc(NCCCc2ccc(-c3ccc(F)cc3)cc2)c2nc(C)sc2n1] | LM 13029 | Method A 2 h (60° C.) | 99% | Cs₂CO₃ 2 h (90° C.) | 50% | N-(3-(4′-fluoro- [1,1′-biphenyl]- 4-yl)propyl)-2- methyl-5- (methylsulfanyl)- [1,3]thiazolo[5, 4-d]pyrimidin- 7-amine | ¹H NMR (399 MHz, CHLOROFORM-d) δ 7.50-7.57 (m, 2H), 7.48 (d, J = 8.17 Hz, 2H), 7.26-7.29 (m, 2H), 7.12 (t, J = 8.56 Hz, 2H), 5.96 (br. s., 1H), 3.69 (q, J = 6.62 Hz, 2H), 2.79 (t, J = 7.59 Hz, 2H), 2.72 (s, 3H), 2.54 (s, 3H), 2.06 (quin, J = |
| | | | | | | | | 7.20 Hz, 2H); LCMS: ESI |
| | | | | | | | | [M + H]+ = 425.0 |
| 358 | 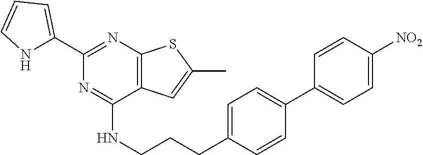 [SMILES: Cc1cc2c(NCCCc3ccc(-c4ccc([N+](=O)[O-])cc4)cc3)nc(-c3ccc[nH]3)nc2s1] | LM 16046 | Method D 1 d (60° C.) | 88% | Cs₂CO₃ 2 h (90° C.) | 49% | 6-methyl-N-(3- (4′-nitro-[1,1′- biphenyl]-4- yl)propyl)-2- (1H-pyrrol-2- yl)thieno[2,3- d]pyrimidin-4- amine | ¹H NMR (399 MHz, CHLOROFORM-d) δ 9.44 (br. s., 1H), 8.28 (d, J = 8.95 Hz, 2H), 7.71 (d, J = 8.56 Hz, 2H), 7.56 (d, J = 8.17 Hz, 2H), 7.34 (d, J = 8.17 Hz, 2H), 7.01 (br. s., 1H), 6.84 (br. s., 1H), 6.59 (s, 1H), 6.27 (d, J = 3.11 Hz, 1H), 4.97 (br. s., 1H), 3.73 (q, J = 6.62 Hz, 2H), 2.83 (t, J = 7.20 Hz, 2H), |
| | | | | | | | | 2.47 (s, 3H), 2.10 (quin, J = 7.10 |
| | | | | | | | | Hz, 2H); LCMS: ESI [M + H]+ = |
| | | | | | | | | 470.1 |
| 359 | 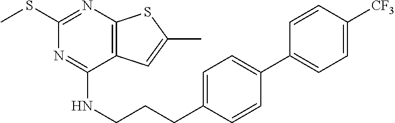 | KH 5002 | Method A 2 h (60° C.) | 89% | Cs₂CO₃ 2 h (90° C.) | 57% | 6-methyl-2- (methylsulfanyl)- N-(3-[4′- (trifluoromethyl)- [1,1′-biphenyl]-4- yl]propyl)thieno [2,3- d]pyrimidin-4- | ¹H NMR (399 MHz, CHLOROFORM-d) δ 7.67 (s, 4H), 7.53 (d, J = 8.17 Hz, 2H), 7.31 (d, J = 7.79 Hz, 2H), 6.51 (s, 1H), 5.03 (br. s., 1H), 3.67 (q, J = 6.62 Hz, 2H), 2.79 (t, J = 7.40 Hz, 2H), 2.53 (s, 3H), 2.42 (s, 3H), 1.97-2.11 (m, 2H); LCMS: |
| | | | | | | | amine | ESI [M + H]+ = 474.1 |
| 360 | 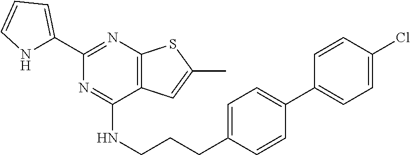 [SMILES: Cc1cc2c(NCCCc3ccc(-c4ccc(Cl)cc4)cc3)nc(-c3ccc[nH]3)nc2s1] | LM 16046 | Method D 1 d (60° C.) | 88% | Cs₂CO₃ 2 h (90° C.) | 45% | N-(3-(4′- chloro-[1,1′- biphenyl]-4- yl)propyl)-6- methyl-2-(1H- pyrrol-2- yl)thieno[2,3- d]pyrimidin-4- amine | ¹H NMR (399 MHz, CHLOROFORM-d) δ 9.41 (br. s., 1H), 7.52 (dd, J = 2.53, 8.37 Hz, 4H), 7.39-7.44 (m, 2H), 7.31 (d, J = 8.17 Hz, 2H), 7.03 (br. s., 1H), 6.85 (br. s., 1H), 6.54 (s, 1H), 6.29 (q, J = 2.98 Hz, 1H), 4.92 (br. s., 1H), 3.74 (q, J = 6.62 Hz, 2H), 2.82 (t, J = 7.20 Hz, 2H), 2.46 (s, 3H), 2.10 (quin, J = |
| | | | | | | | | 7.10 Hz, 2H); LCMS: ESI |
| | | | | | | | | [M + H]+ = 459.1 |
| 361 | 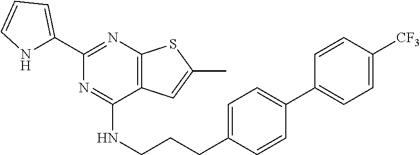 [SMILES: Cc1cc2c(NCCCc3ccc(-c4ccc(C(F)(F)F)cc4)cc3)nc(-c3ccc[nH]3)nc2s1] | LM 16046 | Method D 1 d (60° C.) | 88% | Cs₂CO₃ 4 h (90° C.) | 47% | 6-methyl-2- (1H-pyrrol-2- yl)-N-(3-(4′- (trifluoromethyl)- [1,1′-biphenyl]-4- yl)propyl)thieno [2,3- d]pyrimidin-4- amine | ¹H NMR (399 MHz, CHLOROFORM-d) δ 9.42 (br. s., 1H), 7.70 (s, 4H), 7.56 (d, J = 7.79 Hz, 2H), 7.34 (d, J = 8.17 Hz, 2H), 7.03 (br. s., 1H), 6.85 (br. s., 1H), 6.57 (s, 1H), 6.29 (d, J = 3.50 Hz, 1H), 4.92 (br. s., 1H), 3.75 (q, J = 6.88 Hz, 2H), 2.84 (t, J = 7.20 Hz, 2H), 2.47 (s, 3H), 2.11 (t, J = 7.01 Hz, 2H); |
| | | | | | | | | LCMS: ESI [M + H]+ = 493.1 |
| 362 | 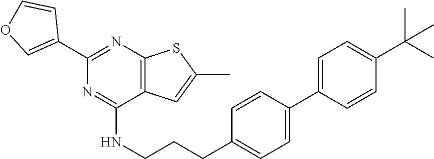 | KH 6067 | Method A 3 d (60° C.) 6 h (90° C.) | 89% | K₂CO₃ ₂ h (90° C.) | 33% | N-(3-(4′-(tert- butyl)-[1,1′- biphenyl]-4- yl)propyl)-2- (furan-3-yl)-6- methylthieno[2, 3-d]pyrimidin- 4-amine | ¹H NMR (399 MHz, CHLOROFORM-d) δ 8.17 (s, 1H), 7.55 (dd, J = 2.14, 8.37 Hz, 4H), 7.43-7.50 (m, 3H), 7.30 (d, J = 8.17 Hz, 2H), 7.04 (d, J = 1.17 Hz, 1H), 6.52 (s, 1H), 4.88 (br. s., 1H), 3.75 (q, J = 6.62 Hz, 2H), 2.83 (t, J = 7.20 Hz, 2H), 2.45 (s, 3H), 2.11 (quin, J = 7.10 Hz, 2H), 1.38 (s, 9H); LCMS: |
| | | | | | | | | ESI [M + H]+ = 482.2 |
| 363 | 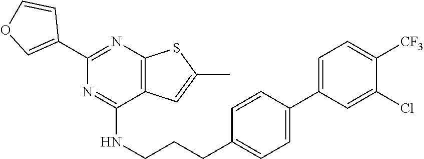 [SMILES: Cc1cc2c(NCCCc3ccc(-c4ccc(C(F)(F)F)c(Cl)c4)cc3)nc(-c3ccoc3)nc2s1] | KH 6067 | Method A 3 d (60° C.) 6 h (90° C.) | 89% | K₂CO₃ 2 h (90° C.) | 65% | N-(3-(3′- chloro-4′- (trifluoromethyl)- [1,1′-biphenyl]-4- yl)propyl)-2- (furan-3-yl)-6- methylthieno[2, 3-d]pyrimidin- 4-amine | ¹H NMR (399 MHz, CHLOROFORM-d) δ 8.13 (s, 1H), 7.69-7.78 (m, 2H), 7.49- 7.58 (m, 3H), 7.45 (s, 1H), 7.33 (d, J = 7.79 Hz, 2H), 7.04 (s, 1H), 6.61 (s, 1H), 4.96 (br. s., 1H), 3.74 (q, J = 6.62 Hz, 2H), 2.83 (t, J = 7.20 Hz, 2H), 2.50 (s, 3H), 2.10 (quin, J = 7.10 Hz, 2H); LCMS: ESI [M + H]+ = 528.1 |
| 364 | 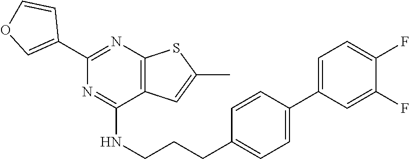 [SMILES: Cc1cc2c(NCCCc3ccc(-c4ccc(F)c(F)c4)cc3)nc(-c3ccoc3)nc2s1] | KH 6067 | Method A 3 d (60° C.) 6 h (90° C.) | 89% | K₂CO₃ 2 h (90° C.) | 56% | N-(3-(3′,4′- difluoro-[1,1′- biphenyl]-4- yl)propyl)-2- (furan-3-yl)-6- methylthieno[2, 3-d]pyrimidin- 4-amine | ¹H NMR (399 MHz, METHANOL-d4) δ 8.02 (s, 1H), 7.38-7.51 (m, 4H), 7.20-7.36 (m, 4H), 6.95 (dd, J = 1.17, 11.29 Hz, 2H), 3.58-3.68 (m, 2H), 2.69-2.81 (m, 2H), 2.49 (d, J = 1.17 Hz, 3H), 1.94-2.09 (m, 2H); LCMS: ESI [M + H]+ = 462.1 |
| 365 | 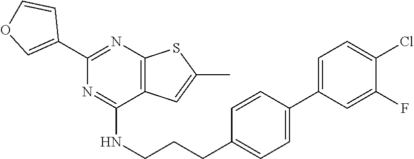 [SMILES: Cc1cc2c(NCCCc3ccc(-c4ccc(Cl)c(F)c4)cc3)nc(-c3ccoc3)nc2s1] | LH 6067 | Method A 3 d (60° C.) 6 h (90° C.) | 89% | K₂CO₃ 2 h (90° C.) | 52% | N-(3-(4′- chloro-3′- fluoro-[1,1′- biphenyl]-4- yl)propyl)-2- (furan-3-yl)-6- methylthieno[2, 3-d]pyrimidin- 4-amine | ¹H NMR (399 MHz, CHLOROFORM-d) δ 8.12 (s, 1H), 7.40-7.51 (m, 4H), 7.27- 7.38 (m, 4H), 7.02 (s, 1H), 6.58 (s, 1H), 4.92 (br. s., 1H), 3.72 (q, J = 6.62 Hz, 2H), 2.81 (t, J = 7.40 Hz, 2H), 2.48 (s, 3H), 2.08 (quin, J = 7.20 Hz, 2H); LCMS: ESI [M + H]+ = 478.1 |
| 366 | 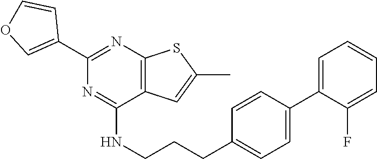 [SMILES: Cc1cc2c(NCCCc3ccc(-c4ccccc4F)cc3)nc(-c3ccoc3)nc2s1] | KH 6067 | Method A 3 d (60° C.) 6 h (90° C.) | 89% | K₂CO₃ 2 h (90° C.) | 68% | N-(3-(2′-fluoro- [1,1′-bipheny]]- 4-yl)propyl)-2- (furan-3-yl)-6- methylthieno[2, 3-d]pyrimidin- 4-amine | ¹H NMR (399 MHz, CHLOROFORM-d) δ 8.17 (s, 1H), 7.40-7.57 (m, 4H), 7.29- 7.36 (m, 3H), 7.13-7.25 (m, 2H), 7.05 (s, 1H), 6.58 (s, 1H), 4.96 (br. s., 1H), 3.75 (q, J = 6.88 Hz, 2H), 2.83 (t, J = 7.20 Hz, 2H), 2.49 (s, 3H), 2.11 (quin, J = 7.10 Hz, 2H); LCMS: ESI [M + H]+ = 444.0 |

### Example 5: Scheme 3 S_{N}Ar+Conversion to Boronate Ester+Suzuki Reaction or Chan Lam

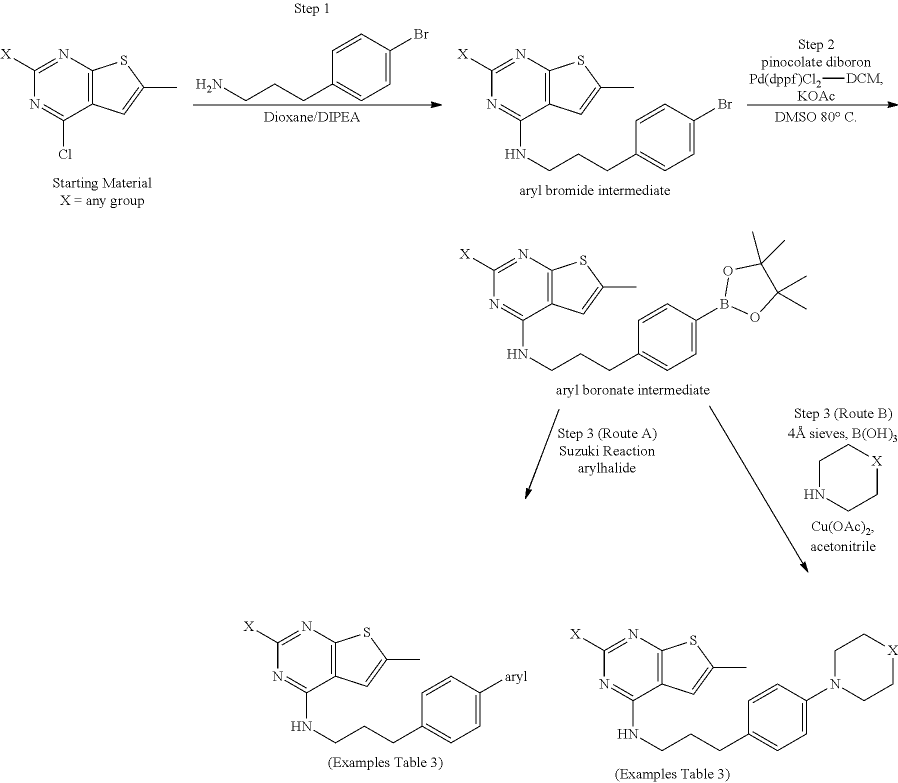

General Procedure for Scheme 3: Reaction conditions and compound data for specific examples are listed in Table 3. Starting materials were synthesized as detailed in the building blocks section.

Step 1: Following the protocol in Scheme 1, Method A (using temperatures of 40° C.-90° C.), the appropriate starting material was reacted with 4-bromophenylpropylamine to give the aryl bromide intermediate for use in step 2.

Step 2: A flask with a stir bar was heated to 250° C. under vacuum for 2 minutes, and then allowed to cool to room temperature under vacuum for an additional 10 minutes, after which time an N₂ atmosphere was continuously maintained. Potassium acetate (4 eqv.) and DMSO (5 mL) were added, followed by the addition of the appropriate arylbromide starting material (1 eqv.) and bis(pinacolato)diboron (2 eqv.) and Pd(dppf)Cl₂-DCM adduct (0.15 eqv). The reaction flask was evacuated and purged 3× with N₂, and stirred for 2 h at 80° C. Upon completion, the reaction was cooled to room temperature. Ice was added, and the reaction was allowed to sit at room temperature for 1 hour, after which the precipitated solids were filtered, washing with cold DI water (3×). The solids were collected and dried in vacuo. Further purification by silica gel column chromatography gave the desired intermediate aryl boronate intermediate (yields for steps 1-2 in Table 3) for use in Step 3 (Routes A or B).

Step 3 (Route A): Following the General Suzuki Protocol, the intermediate boronate ester from step 2 (0.030 mmol) and the appropriate aryl halide (0.060 mmol) were reacted for the time and temperature specified, then purified by prep HPLC or silica gel column chromatography, or a combination of the two, to give the pure product (Table 3).

Step 3 (Route B): 4 Å sieves were activated overnight at 300° C. under vacuum, then cooled to room temperature and kept under a N₂ atmosphere thereafter. To a threaded vial was added the activated 4 Å sieves (10 mg), the intermediate boronate ester derivative from step 2 (0.030 mmol), the appropriate amine (2 eqv.), boric acid (2 eqv.) and Cu(OAc)₂ (0.05 eqv.), followed by anhydrous acetonitrile (0.15 mL). The reaction was sealed under an atmosphere of air and reacted under the conditions specified in Table 3. Upon completion, the reaction was cooled to room temperature and quenched with saturated Na₂S₂O₃(aq) (0.1 mL). The sieves were filtered, and the reaction was concentrated in vacuo, and the reaction was purified by prep HPLC to give the pure product (Table 3).

TABLE 3
Examples from Scheme 3 (Routes A and B)

| | | | Steps | Step 3 | | | |
|---|---|---|---|---|---|---|---|
| Cmpd. | | Starting | 1-2 | Route/ | Step 3 | | |
| No. | Structure | Material | Yield | Conditions | Yield | IUPAC | Compound Data |
| 367 | 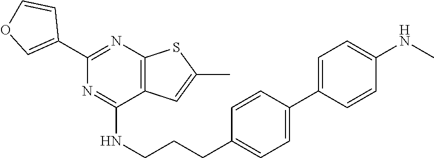 [SMILES: CNc1ccc(-c2ccc(CCCNc3nc(-c4ccoc4)nc4sc(C)cc34)cc2)cc1] | KH 6067 | 85% | Route A Cs₂CO₃ 2 h (50° C.) | 15% | 2-(furan-3-yl)-6- methyl-N-(3-[4′- (methylamino)- [1,1′-biphenyl]-4- yl]propyl)thieno [2,3-d]pyrimidin- 4-amine | ¹H NMR (399 MHz, CHLOROFORM-d) δ 8.16 (br. s., 1H), 7.40-7.58 (m, 5H), 7.27 (d, J = 1.95 Hz, 5H), 7.03 (br. s., 1H), 6.70 (d, J = 7.01 Hz, 2H), 6.49 (br. s., 1H), 4.85 (br. s., 1H), 3.69-3.81 (m, 2H), 2.89 (d, J = 1.95 Hz, 2H), 2.81 (t, J = 7.01 Hz, 2H), 2.45 (s, 2H), 2.09 (t, J = 6.62 Hz, 2H); LCMS: ESI [M + H]⁺ = 455.2 |
| 368 | 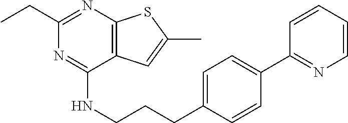 [SMILES: CCc1nc(NCCCc2ccc(-c3ccccn3)cc2)c2cc(C)sc2n1] | KH 4007 | 67% | Route A Cs₂CO₃ 2 h (80° C.) | 71% | 2-ethyl-6-methyl- N-(3-(4-(pyridin- 2- yl)phenyl)propyl) thieno[2,3-d] pyrimidin-4- amine | ¹H NMR (400 MHz, CHLOROFORM-d) δ 8.72-8.65 (m, 1H), 7.94 (dd, J = 8.5, 2.0 Hz, 2H), 7.73 (qdd, J = 8.0, 4.0, 1.8 Hz, 2H), 7.34 (dd, J = 8.6, 2.4 Hz, 2H), 7.29-7.19 (m, 1H), 6.53 (s, 1H), 3.69 (q, J = 6.7, 5.4 Hz, 2H), 2.84 (ddt, J = 9.2, 7.4, 3.1 Hz, 4H), 2.47-2.40 (m, 3H), 2.08 (dq, J = 14.3, 7.2 Hz, 2H), 1.35 (td, J = 7.8, 3.0 Hz, 3H); |
| | | | | | | | LCMS: ESI [M + H]⁺ = 389.2 |
| 369 | 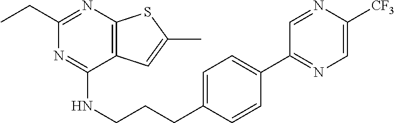 [SMILES: CCc1nc(NCCCc2ccc(-c3cnc(C(F)(F)F)cn3)cc2)c2cc(C)sc2n1] | KH 4007 | 67% | Route A Cs₂CO₃ 2 h (80° C.) | 35% | 2-ethyl-6-methyl- N-(3-(4-(5- (trifluoromethyl) pyrazin-2- yl)phenyl) propyl)thieno[2,3- d]pyrimidin-4- amine | ¹H NMR (400 MHz, CHLOROFORM-d) δ 9.09 (d, J = 1.5 Hz, 1H), 8.97 (d, J = 1.5 Hz, 1H), 8.04-7.95 (m, 2H), 7.40 (d, J = 8.3 Hz, 2H), 6.65 (s, 1H), 3.71 (q, J = 6.6 Hz, 2H), 2.90-2.80 (m, 4H), 2.49 (s, 3H), 2.09 (q, J = 7.3 Hz, 2H), 1.35 (t, J = 7.5 Hz, 3H); LCMS: ESI [M + H]⁺ = 458.2 |
| 370 | 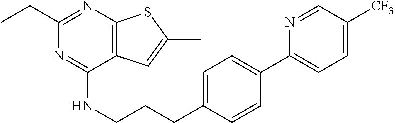 [SMILES: CCc1nc(NCCCc2ccc(-c3ccc(C(F)(F)F)cn3)cc2)c2cc(C)sc2n1] | KH 4007 | 67% | Route A Cs₂CO₃ 2 h (80° C.) | 44% | 2-ethyl-6-methyl- N-(3-(4-(5- (trifluoromethyl) pyridin-2- yl)phenyl) propyl)thieno[2,3- d]pyrimidin-4- amine | ¹H NMR (400 MHz, CHLOROFORM-d) δ 8.94 (br. s., 1H), 7.98 (d, J = 8.22 Hz, 3H), 7.83 (d, J = 8.22 Hz, 1H), 7.37 (d, J = 7.83 Hz, 2H), 6.59 (br. s., 1H), 3.71 (d, J = 6.26 Hz, 2H), 2.75-2.92 (m, 4H), 2.47 (s, 3H), 1.99-2.20 (m, 2H), 1.36 (t, J = 7.63 Hz, 3H); LCMS: ESI [M + H]⁺ = 457.2 |
| 371 | 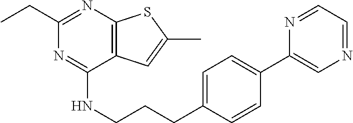 [SMILES: CCc1nc(NCCCc2ccc(-c3cnccn3)cc2)c2cc(C)sc2n1] | KH 4007 | 67% | Route A Cs₂CO₃ 2 h (80° C.) | 32% | 2-ethyl-6-methyl- N- (3-(4-(pyrazin-2- yl)phenyl)propyl) thieno[2,3- d]pyrimidin-4- amine | ¹H NMR (400 MHz, CHLOROFORM-d) δ 8.95-9.07 (m, 1H), 8.63 (d, J = 1.57 Hz, 1H), 8.44-8.54 (m, 1H), 7.90-8.03 (m, 2H), 7.32-7.48 (m, 2H), 6.60 (s, 1H), 3.60-3.80 (m, 2H), 2.76-2.93 (m, 4H), 2.37-2.53 (m, 3H), 1.99-2.18 (m, 2H), 1.36 (t, J = 7.43 Hz, 3H); LCMS: ESI [M + H]⁺ = 390.2 |
| 372 | 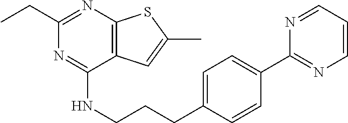 [SMILES: CCc1nc(NCCCc2ccc(-c3ncccn3)cc2)c2cc(C)sc2n1] | KH 4007 | 67% | Route A Cs₂CO₃ 2 h (80° C.) | 38% | 2-ethyl-6-methyl- N-(3- (4-(pyrimidin-2- yl)phenyl)propyl) thieno[2,3- d]pyrimidin- 4-amine | ¹H NMR (400 MHz, CHLOROFORM-d) δ 8.72 (d, J = 9.00 Hz, 2H), 8.29 (br. s., 2H), 7.03-7.32 (m, 3H), 6.46 (br. s., 1H), 3.61 (br. s., 2H), 2.75 (br. s., 4H), 2.34 (br. s., 3H), 2.00 (br. s., 2H), 1.27 (br. s., 3H); LCMS: ESI [M + H]⁺ = 390.2 |
| 373 | 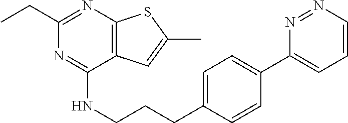 [SMILES: CCc1nc(NCCCc2ccc(-c3cccnn3)cc2)c2cc(C)sc2n1] | KH 4007 | 67% | Route A Cs₂CO₃ 2 h (80° C.) | 37% | 2-ethyl-6-methyl- N-(3-(4- (pyridazin-3- yl)phenyl)propyl) thieno[2,3-d] pyrimidin-4- amine | ¹H NMR (400 MHz, METHANOL-d₄) δ 9.13 (dd, J = 1.34, 4.89 Hz, 1H), 8.13 (dd, J = 1.47, 8.68 Hz, 1H), 7.98 (d, J = 8.19 Hz, 2H), 7.78 (dd, J = 4.89, 8.68 Hz, 1H), 7.42 (d, J = 8.19 Hz, 2H), 7.00 (d, J = 1.22 Hz, 1H), 3.66 (t, J = 7.03 Hz, 2H), 2.84 (t, J = 7.52 Hz, 2H), 2.75 (q, J = 7.58 Hz, 2H), 2.51 (s, 3H), 2.10 |
| | | | | | | | (quin, J = 7.24 Hz, 2H), 1.32 (t, J = 7.58 |
| | | | | | | | Hz, 3H); LCMS: ESI [M + H]⁺ = 390.2 |
| 374 | 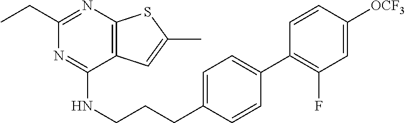 [SMILES: CCc1nc(NCCCc2ccc(-c3ccc(OC(F)(F)F)cc3F)cc2)c2cc(C)sc2n1] | KH 4007 | 67% | Route A Cs₂CO₃ 3 h (80° C.) | 29% | 2-ethyl-N-(3-(2′- fluoro-4′-(tri- fluoromethoxy)- [1,1′-biphenyl]-4- yl)propyl)-6- methylthieno[2,3- d]pyrimidin-4- amine | 1H NMR (400 MHz, CHLOROFORM-d) δ 7.41-7.50 (m, 3H), 7.33 (d, J = 8.07 Hz, 2H), 7.09 (t, J = 10.76 Hz, 2H), 6.61 (s, 1H), 3.72 (q, J = 6.68 Hz, 2H), 2.85 (quin, J = 7.55 Hz, 4H), 2.50 (s, 3H), 2.09 (quin, J = 7.12 Hz, 2H), 1.36 (t, J = 7.58 Hz, 3H); LCMS: ESI [M + H]⁺ = 490.2 |
| 375 | 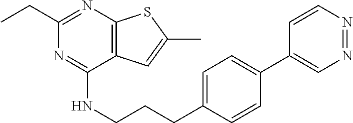 [SMILES: CCc1nc(NCCCc2ccc(-c3ccnnc3)cc2)c2cc(C)sc2n1] | KH 4007 | 67% | Route A Cs₂CO₃ 2 h (80° C.) | 56% | 2-ethyl-6-methyl- N-(3-(4- (pyridazin-4- yl)phenyl)propyl) thieno[2,3- d]pyrimidin- 4-amine | ¹H NMR (400 MHz, METHANOL-d4) δ 9.39 (d, J = 1.10 Hz, 1H), 9.07 (d, J = 5.38 Hz, 1H), 7.85 (dd, J = 2.45, 5.50 Hz, 1H), 7.63 (d, J = 8.19 Hz, 2H), 7.32 (d, J = 8.07 Hz, 2H), 6.87 (d, J = 0.86 Hz, 1H), 3.54 (t, J = 6.91 Hz, 2H), 2.71 (t, J = 7.52 Hz, 2H), 2.62 (q, J = 7.58 Hz, 2H), 2.39 (s, 3H), 1.98 (quin, J = |
| | | | | | | | 7.49 Hz, 2H), 1.20 (t, J = 7.58 Hz, 3H); |
| | | | | | | | LCMS: ESI [M + H]⁺ = 390.2 |
| 376 | 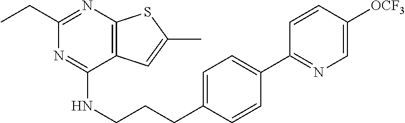 [SMILES: CCc1nc(NCCCc2ccc(-c3ccc(OC(F)(F)F)cn3)cc2)c2cc(C)sc2n1] | KH 4007 | 67% | Route A Cs₂CO₃ 2 h (80° C.) | 54% | 2-ethyl-6-methyl- N-(3-(4-(5- (trifluoro- methoxy)pyridin- 2-yl)phenyl) propyl)thieno [2,3-d]pyrimidin- 4-amine | ¹H NMR (400 MHz, CHLOROFORM-d) δ 8.61 (d, J = 2.08 Hz, 1H), 7.92 (d, J = 8.19 Hz, 2H), 7.76 (d, J = 8.80 Hz, 1H), 7.57-7.66 (m, 1H), 7.35 (d, J = 8.07 Hz, 2H), 6.57 (s, 1H), 3.70 (q, J = 6.52 Hz, 2H), 2.77-2.91 (m, 4H), 2.45 (s, 3H), 2.08 (quin, J = 7.15 Hz, 2H), 1.36 (t, J = 7.58 Hz, 3H); LCMS: ESI [M + H]⁺ = 473.2 |
| 377 | 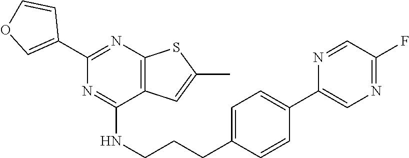 [SMILES: Cc1cc2c(NCCCc3ccc(-c4cnc(F)cn4)cc3)nc(-c3ccoc3)nc2s1] | KH 6067 | 85% | Route A Cs₂CO₃ 2 h (80° C.) | 30% | N-(3-[4-(5- fluoropyrazin- 2-yl)phenyl] propyl)-2- (furan-3-yl)-6- methylthieno [2,3-d]pyrimidin- 4-amine | ¹H NMR (399 MHz, METHANOL-d4) δ 8.65 (s, 1H), 8.51 (d, J = 8.18 Hz, 1H), 8.02 (s, 1H), 7.90 (d, J = 7.79 Hz, 2H), 7.47 (s, 1H), 7.34 (d, J = 7.79 Hz, 2H), 6.87-7.02 (m, 2H), 3.65 (t, J = 6.81 Hz, 2H), 2.79 (t, J = 7.20 Hz, 2H), 2.49 (s, 3H), 2.06 (quin, J = 7.10 Hz, 2H); LCMS: ESI [M + H]⁺ = 446.1 |
| 378 | 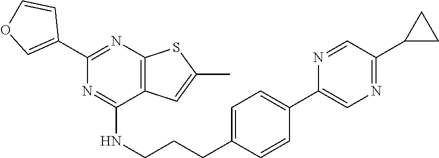 [SMILES: Cc1cc2c(NCCCc3ccc(-c4cnc(C5CC5)cn4)cc3)nc(-c3ccoc3)nc2s1] | KH 6067 | 85% | Route A Cs₂CO₃ 2 h (80° C.) | 22% | N-(3-[4-(5- cyclopropyl- pyrazin-2- yl)phenyl]propyl)- 2-(furan-3-yl)-6- methylthieno[2,3- d]pyrimidin-4- amine | ¹H NMR (399 MHz, METHANOL-d4) δ 8.81 (s, 1H), 8.52 (d, J = 4.28 Hz, 1H), 8.04 (s, 1H), 7.87 (d, J = 8.17 Hz, 2H), 7.47 (s, 1H), 7.34 (d, J = 8.17 Hz, 2H), 6.99 (s, 1H), 6.93 (s, 1H), 3.67 (t, J = 7.01 Hz, 2H), 2.80 (t, J = 7.20 Hz, 2H), 2.50 (s, 3H), 2.18 (br. s., 1H), 2.07 (t, J = 7.40 Hz, 2H), 1.01-1.12 (m, 4H); LCMS: ESI [M + H]+ = 468.2 |
| 379 | 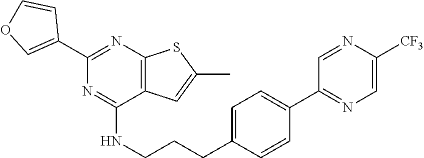 [SMILES: Cc1cc2c(NCCCc3ccc(-c4cnc(C(F)(F)F)cn4)cc3)nc(-c3ccoc3)nc2s1] | KH 6067 | 85% | Route A Cs₂CO₃ 3 h (95° C.) | 40% | 2-(furan-3-yl)-6- methyl-N-(3-(4- [5- (trifluoromethyl) pyrazin-2- yl]phenyl)propyl) thieno[2,3- d]pyrimidin-4- amine | ¹H NMR (399 MHz, ACETONITRILE- d3) δ 8.97-9.22 (m, 1H), 8.49 (d, J = 15.18 Hz, 1H), 8.03-8.15 (m, 2H), 7.48- 7.55 (m, 1H), 7.40-7.47 (m, 1H), 7.34 (d, J = 8.56 Hz, 1H), 7.12 (d, J = 8.17 Hz, 1H), 6.89-7.00 (m, 2H), 6.11-6.26 (m, 1H), 3.59-3.72 (m, 2H), 2.73-2.88 (m, 2H), 2.47-2.59 (m, 3H), 2.05 (dd, J = 7.01, 14.40 Hz, 2H); LCMS: ESI [M + H]+ = 496.1 |
| 380 | 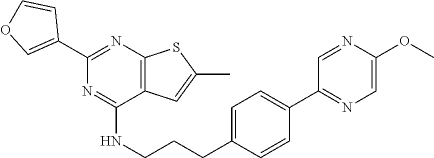 [SMILES: COc1cnc(-c2ccc(CCCNc3nc(-c4ccoc4)nc4sc(C)cc34)cc2)cn1] | KH 6067 | 85% | Route A Cs₂CO₃ 2 h (70° C.) 2 h (80° C.) | 31% | 2-(furan-3-yl)-N- (3-[4-(5- methoxypyrazin- 2- yl)phenyl]propyl)- 6- methylthieno[2,3- d]pyrimidin-4- amine | ¹H NMR (399 MHz, ACETONITRILE- d3) δ 8.55 (s, 1H), 8.23 (s, 1H), 8.07 (s, 1H), 7.86 (d, J = 8.18 Hz, 2H), 7.44-7.50 (m, 1H), 7.33 (d, J = 8.17 Hz, 2H), 6.86- 6.95 (m, 2H), 6.11 (br. s., 1H), 3.95 (s, 3H), 3.62 (q, J = 6.49 Hz, 2H), 2.76 (t, J = 7.59 Hz, 2H), 2.48 (s, 3H), 1.98-2.05 (m, 2H); LCMS: ESI [M + H]+ = 458.1 |
| 381 | 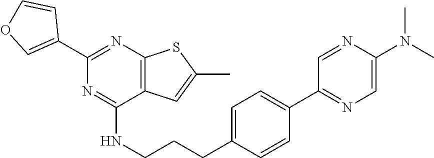 [SMILES: Cc1cc2c(NCCCc3ccc(-c4cnc(N(C)C)cn4)cc3)nc(-c3ccoc3)nc2s1] | KH 6067 | 85% | Route A Cs₂CO₃ 2 h (70° C.) 0.5 h (80° C.) | 24% | 5-[4-(3-([2- (furan-3-yl)-6- methylthieno[2,3- d]pyrimidin-4- yl]amino)propyl) phenyl]-N,N- dimethyl-pyrazin- 2-amine | ¹H NMR (399 MHz, ACETONITRILE- d3) δ 8.51 (s, 1H), 8.10 (d, J = 5.06 Hz, 2H), 7.84 (d, J = 8.17 Hz, 2H), 7.50 (s, 1H), 7.31 (d, J = 8.17 Hz, 2H), 6.89-6.98 (m, 2H), 6.13 (br. s., 1H), 3.59-3.68 (m, 2H), 3.08-3.18 (m, 6H), 2.76 (t, J = 7.40 Hz, 2H), 2.51 (s, 3H), 1.98-2.05 (m, 2H); LCMS: ESI [M + H]+ = 471.2 |
| 382 | 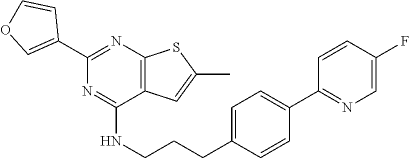 [SMILES: Cc1cc2c(NCCCc3ccc(-c4ccc(F)cn4)cc3)nc(-c3ccoc3)nc2s1] | KH 6067 | 85% | Route A Cs₂CO₃ 2 h (90° C.) 1 h (95° C.) | 41% | N-(3-[4-(5- fluoropyridin-2- yl)phenyl]propyl)- 2-(furan-3-yl)-6- methylthieno[2,3- d]pyrimidin-4- amine | ¹H NMR (399 MHz, ACETONITRILE- d3) δ 8.52 (d, J = 3.11 Hz, 1H), 8.10 (s, 1H), 7.92 (d, J = 8.17 Hz, 2H), 7.85 (dd, J = 4.48, 8.76 Hz, 1H), 7.59 (dt, J = 3.11, 8.76 Hz, 1H), 7.46-7.51 (m, 1H), 7.35 (d, J = 8.17 Hz, 2H), 6.88-6.97 (m, 2H), 6.15 (br. s., 1H), 3.65 (q, J = 6.62 Hz, 2H), 2.80 (t, J = 7.59 Hz, 2H), 2.51 (d, J = 0.78 Hz, 3H), 2.00-2.07 (m, 2H); LCMS: ESI [M + H]+ = 445.1 |
| 383 | 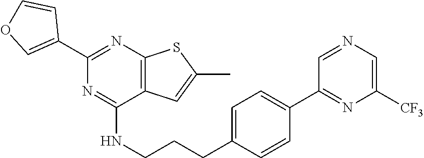 [SMILES: Cc1cc2c(NCCCc3ccc(-c4cncc(C(F)(F)F)n4)cc3)nc(-c3ccoc3)nc2s1] | KH 6067 | 85% | Route A Cs₂CO₃ 2 h (90° C.) 3 h (95° C.) | 37% | 2-(furan-3-yl)-6- methyl-N-(3-(4- [6- (trifluoromethyl) pyrazin-2-yl] phenyl)propyl) thieno[2,3- d]pyrimidin-4- amine | ¹H NMR (399 MHz, ACETONITRILE-d3) δ 9.31 (s, 1H), 8.91 (s, 1H), 8.08 (s, 1H), 8.02 (d, J = 7.79 Hz, 2H), 7.49 (s, 1H), 7.43 (d, J = 7.79 Hz, 2H), 6.86-6.97 (m, 2H), 6.15 (br. s., 1H), 3.65 (q, J = 6.36 Hz, 2H), 2.82 (t, J = 7.40 Hz, 2H), 2.50 (s, 3H), 2.01-2.08 (m, 2H); LCMS: ESI [M + H]+ = 496.0 |
| 384 | 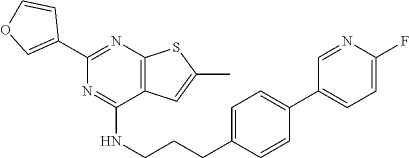 [SMILES: Cc1cc2c(NCCCc3ccc(-c4ccc(F)nc4)cc3)nc(-c3ccoc3)nc2s1] | KH 6067 | 85% | Route A Cs₂CO₃ 2.5 h (90° C.) | 33% | N-(3-[4-(6- fluoropyridin-3- yl)phenyl]propyl)- 2-(furan-3-yl)-6- methylthieno[2,3- d]pyrimidin-4- amine | ¹H NMR (399 MHz, ACETONITRILE-d3) δ 8.42 (s, 1H), 8.06-8.15 (m, 2H), 7.47- 7.57 (m, 3H), 7.32-7.39 (m, 2H), 7.08 (dd, J = 2.14, 8.37 Hz, 1H), 6.89-6.96 (m, 2H), 6.15 (br. s., 1H), 3.59-3.69 (m, 2H), 2.78 (t, J = 7.40 Hz, 2H), 2.51 (s, 3H), 2.00-2.07 (m, 2H); LCMS: ESI [M + H]+ = 445.1 |
| 385 | 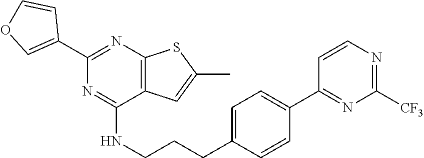 [SMILES: Cc1cc2c(NCCCc3ccc(-c4ccnc(C(F)(F)F)n4)cc3)nc(-c3ccoc3)nc2s1] | KH 6067 | 85% | Route A Cs₂CO₃ 2.5 h (90° C.) | 38% | 2-(furan-3-yl)-6- methyl-N-(3- (4-[2- (trifluoromethyl) pyrimidin-4- yl]phenyl)propyl) thieno[2,3- d]pyrimidin-4- amine | ¹H NMR (399 MHz, ACETONITRILE-d3) δ 8.91 (d, J = 5.06 Hz, 1H), 8.07-8.17 (m, 3H), 7.98-8.05 (m, 1H), 7.49 (t, J = 1.56 Hz, 1H), 7.38-7.45 (m, 2H), 6.84-6.96 (m, 2H), 6.14 (br. s., 1H), 3.64 (q, J = 6.49 Hz, 2H), 2.82 (t, J = 7.40 Hz, 2H), 2.49 (s, 3H), 2.00-2.09 (m, 2H); LCMS: ESI [M + H]+ = 496.1 |
| 386 | 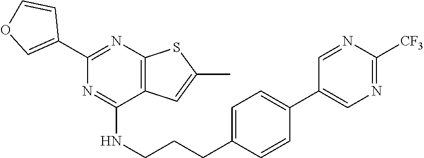 [SMILES: Cc1cc2c(NCCCc3ccc(-c4cnc(C(F)(F)F)nc4)cc3)nc(-c3ccoc3)nc2s1] | KH 6067 | 85% | Route A Cs₂CO₃ 4 h (90° C.) | 19% | 2-(furan-3-yl)-6- methyl-N-(3-(4- [2- trifluoromethyl) pyrimidin-5- yl]phenyl)propyl) thieno[2,3- d]pyrimidin-4- amine | ¹H NMR (399 MHz, ACETONITRILE-d3) δ 9.16 (s, 2H), 8.07 (s, 1H), 7.67 (d, J = 8.17 Hz, 2H), 7.51 (t, J = 1.75 Hz, 1H), 7.44 (d, J = 8.17 Hz, 2H), 6.94 (dd, J = 0.97, 10.71 Hz, 2H), 6.16 (br. s., 1H), 3.66 (q, J = 6.75 Hz, 2H), 2.82 (t, J = 7.59 Hz, 2H), 2.51 (s, 3H), 2.03-2.07 (m, 2H); LCMS: ESI [M + H]+ = 496.1 |
| 387 | 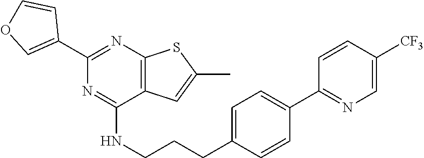 [SMILES: Cc1cc2c(NCCCc3ccc(-c4ccc(C(F)(F)F)cn4)cc3)nc(-c3ccoc3)nc2s1] | KH 6067 | 85% | Route A Cs₂CO₃ 4 h (90° C.) | 23% | 2-(furan-3-yl)-6- methyl-N- (3-(4-[5- (trifluoromethyl) pyridin-2- yl]phenyl)propyl) thieno[2,3- d]pyrimidin-4- amine | ¹H NMR (399 MHz, ACETONITRILE-d3) δ 8.94 (s, 1H), 8.07-8.13 (m, 2H), 8.03 (d, J = 7.79 Hz, 2H), 7.98 (d, J = 8.17 Hz, 1H), 7.49 (t, J = 1.75 Hz, 1H), 7.39 (d, J = 8.18 Hz, 2H), 6.88-6.97 (m, 2H), 6.15 (br. s., 1H), 3.65 (q, J = 6.62 Hz, 2H), 2.81 (t, J = 7.40 Hz, 2H), 2.50 (s, 3H), 2.01- 2.07 (m, 2H); LCMS: ESI [M + H]+ = 495.1 |
| 388 | 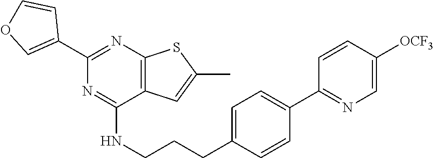 [SMILES: Cc1cc2c(NCCCc3ccc(-c4ccc(OC(F)(F)F)cn4)cc3)nc(-c3ccoc3)nc2s1] | KH 6067 | 85% | Route A Cs₂CO₃ 3 h (90° C.) | 32% | 2-(furan-3-yl)-6- methyl-N-(3-(4- [5-(trifluoro- methoxy)pyridin- 2-yl]phenyl) propyl)thieno[2,3- d]pyrimidin-4- amine | ¹H NMR (399 MHz, ACETONITRILE-d3) δ 8.61 (d, J = 1.95 Hz, 1H), 8.09 (s, 1H), 7.95 (d, J = 7.79 Hz, 2H), 7.90 (d, J = 8.95 Hz, 1H), 7.75 (d, J = 8.95 Hz, 1H), 7.49 (s, 1H), 7.36 (d, J = 8.17 Hz, 2H), 6.92 (d, J = 12.85 Hz, 2H), 6.13 (br. s., 1H), 3.64 (q, J = 6.62 Hz, 2H), 2.79 (t, J = 7.59 Hz, 2H), 2.50 (s, 3H), 1.98-2.09 (m, 2H); LCMS: ESI [M + H]+ = 511.0 |
| 389 | 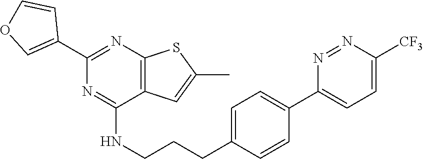 [SMILES: Cc1cc2c(NCCCc3ccc(-c4ccc(C(F)(F)F)nn4)cc3)nc(-c3ccoc3)nc2s1] | KH 6067 | 85% | Route A Cs₂CO₃ 3 h (90° C.) | 34% | 2-(furan-3-yl)-6- methyl-N-(3-(4- [6-(trifluoro- methyl)pyridazin- 3-yl]phenyl) propyl)thieno[2,3- d]pyrimidin-4- amine | ¹H NMR (399 MHz, ACETONITRILE-d3) δ 8.21 (d, J = 8.95 Hz, 1H), 8.06-8.13 (m, 3H), 8.02 (d, J = 8.95 Hz, 1H), 7.42-7.54 (m, 3H), 6.93 (d, J = 8.18 Hz, 2H), 6.16 (br. s., 1H), 3.67 (q, J = 6.49 Hz, 2H), 2.85 (t, J = 7.40 Hz, 2H), 2.51 (s, 3H), 2.03- 2.09 (m, 2H); LCMS: ESI [M + H]+ = 496.1 |
| 390 | 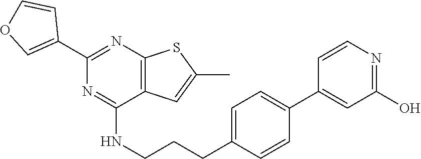 [SMILES: Cc1cc2c(NCCCc3ccc(-c4ccnc(O)c4)cc3)nc(-c3ccoc3)nc2s1] | KH 6067 | 85% | Route A Cs₂CO₃ 4.5 h (100° C.) | 10% | 4-[4-(3-([2-(furan- 3-yl)-6- methylthieno[2,3- d]pyrimidin-4- yl]amino)propyl) phenyl]pyridin- 2-ol | ¹H NMR (399 MHz, METHANOL-d4) δ 8.03 (s, 1H), 7.55 (d, J = 7.79 Hz, 2H), 7.44-7.51 (m, 2H), 7.33 (d, J = 8.17 Hz, 2H), 6.98 (s, 1H), 6.93 (s, 1H), 6.66-6.74 (m, 2H), 3.66 (t, J = 7.01 Hz, 2H), 2.79 (t, J = 7.20 Hz, 2H), 2.51 (s, 3H), 2.07 (quin, J = 7.10 Hz, 2H); LCMS: ESI [M + H]+ = 443.1 |
| 391 | 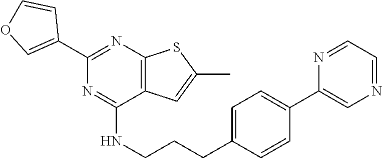 [SMILES: Cc1cc2c(NCCCc3ccc(-c4cnccn4)cc3)nc(-c3ccoc3)nc2s1] | KH 6067 | 85% | Route A Cs₂CO₃ 3 h (90° C.) | 40% | 2-(furan-3-yl)-6- methyl-N-(3-[4- (pyrazin-2- yl)phenyl]propyl) thieno[2,3- d]pyrimidin-4- amine | ¹H NMR (399 MHz, ACETONITRILE-d3) δ 9.06 (s, 1H), 8.60 (s, 1H), 8.49 (d, J = 1.95 Hz, 1H), 8.09 (s, 1H), 7.99 (d, J = 8.17 Hz, 2H), 7.49 (s, 1H), 7.40 (d, J = 8.17 Hz, 2H), 6.93 (d, J = 9.73 Hz, 2H), 6.15 (br. s., 1H), 3.66 (q, J = 6.75 Hz, 2H), 2.81 (t, J = 7.40 Hz, 2H), 2.51 (s, 3H), 2.02-2.07 (m, 2H); LCMS: ESI [M + H]+ = 428.1 |
| 392 | 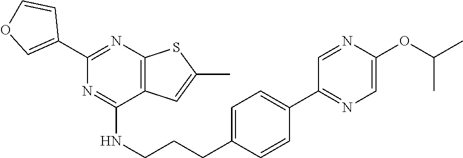 [SMILES: Cc1cc2c(NCCCc3ccc(-c4cnc(OC(C)C)cn4)cc3)nc(-c3ccoc3)nc2s1] | KH 6067 | 85% | Route A Cs₂CO₃ 2.5 h (90° C.) | 12% | 2-(furan-3-yl)-6- methyl-N-(3- (4-[5-(propan-2- yloxy)pyrazin- 2-yl]phenyl) propyl)thieno[2,3- d]pyrimidin-4- amine | ¹H NMR (399 MHz, ACETONITRILE-d3) δ 8.56 (d, J = 1.17 Hz, 1H), 8.17 (d, J = 1.17 Hz, 1H), 8.11 (s, 1H), 7.89 (d, J = 8.17 Hz, 2H), 7.51 (s, 1H), 7.36 (d, J = 8.17 Hz, 2H), 6.94 (dd, J = 1.17, 9.34 Hz, 2H), 6.16 (br. s., 1H), 5.32 (td, J = 6.23, 12.46 Hz, 1H), 3.66 (q, J = 6.75 Hz, 2H), 2.80 (t, J = 7.59 Hz, 2H), 2.52 (s, 3H), 2.01-2.09 (m, 2H), 1.37 (d, J = 6.23 Hz, 6H); LCMS: ESI [M + H]+ = 486.2 |
| 393 | 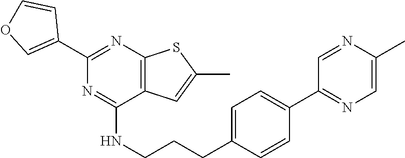 [SMILES: Cc1cnc(-c2ccc(CCCNc3nc(-c4ccoc4)nc4sc(C)cc34)cc2)cn1] | KH 6067 | 85% | Route A Cs₂CO₃ 2.5 h (90° C.) | 52% | 2-(furan-3-yl)-6- methyl-N-(3- [4-(5-methyl- pyrazin-2-yl) phenyl]propyl) thieno[2,3-d] pyrimidin-4- amine | ¹H NMR (399 MHz, ACETONITRILE-d3) δ 8.92 (s, 1H), 8.49 (s, 1H), 8.10 (s, 1H), 7.95 (d, J = 7.40 Hz, 2H), 7.50 (t, J = 1.56 Hz, 1H), 7.37 (d, J = 7.40 Hz, 2H), 6.89- 6.96 (m, 2H), 6.17 (br. s., 1H), 3.65 (q, J = 6.23 Hz, 2H), 2.80 (t, J = 7.40 Hz, 2H), 2.52 (d, J = 11.29 Hz, 6H), 1.99-2.08 (m, 2H); LCMS: ESI [M + H]+ = 442.1 |
| 394 | 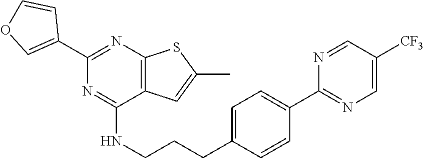 [SMILES: Cc1cc2c(NCCCc3ccc(-c4ncc(C(F)(F)F)cn4)cc3)nc(-c3ccoc3)nc2s1] | KH 6067 | 85% | Route A Cs₂CO₃ 2.5 h (90° C.) | 26% | 2-(furan-3-yl)-6- methyl-N-(3-(4- [5- (trifluoromethyl) pyrimidin-2- yl]phenyl)propyl) thieno[2,3-d] pyrimidin-4- amine | ¹H NMR (399 MHz, ACETONITRILE-d3) δ 9.11 (s, 2H), 8.40 (d, J = 7.79 Hz, 2H), 8.10 (s, 1H), 7.49 (s, 1H), 7.42 (d, J = 8.17 Hz, 2H), 6.93 (d, J = 6.62 Hz, 2H), 6.19 (br. s., 1H), 3.66 (q, J = 6.49 Hz, 2H), 2.84 (t, J = 7.40 Hz, 2H), 2.51 (s, 3H), 2.06 (t, J = 7.20 Hz, 2H); LCMS: ESI [M + H]+ = 496.1 |
| 395 | 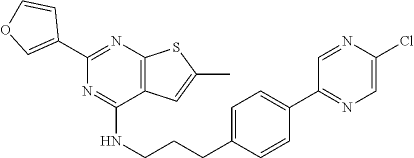 [SMILES: Cc1cc2c(NCCCc3ccc(-c4cnc(Cl)cn4)cc3)nc(-c3ccoc3)nc2s1] | KH 6067 | 85% | Route A Cs₂CO₃ 3 h (90° C.) | 28% | N-(3-[4-(5- chloropyrazin-2- yl)phenyl] propyl)-2-(furan- 3-yl)-6- methylthieno[2,3- d]pyrimidin-4- amine | ¹H NMR (399 MHz, ACETONITRILE-d3) δ 8.84 (d, J = 1.17 Hz, 1H), 8.65 (d, J = 0.78 Hz, 1H), 8.09 (d, J = 0.78 Hz, 1H), 7.96 (d, J = 8.17 Hz, 2H), 7.48-7.51 (m, 1H), 7.40 (d, J = 8.18 Hz, 2H), 6.88-6.96 (m, 2H), 6.15 (br. s., 1H), 3.65 (q, J = 6.49 Hz, 2H), 2.81 (t, J = 7.40 Hz, 2H), 2.51 (s, 3H), 2.00-2.07 (m, 2H); LCMS: ESI [M + H]+ = 462.1 |
| 396 | 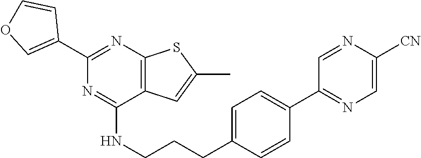 [SMILES: Cc1cc2c(NCCCc3ccc(-c4cnc(C#N)cn4)cc3)nc(-c3ccoc3)nc2s1] | KH 6067 | 85% | Route A Cs₂CO₃ 3 h (90° C.) | 9% | 5-[4-(3-([2- (furan-3-yl)-6- methylthieno[2,3- d]pyrimidin-4- yl]amino)propyl) phenyl]pyrazine- 2-carbonitrile | 1H NMR (399 MHz, METHANOL-d4) δ 9.24 (s, 1H), 9.01 (s, 1H), 8.09 (d, J = 7.79 Hz, 2H), 8.04 (s, 1H), 7.50 (s, 1H), 7.42 (d, J = 8.17 Hz, 2H), 6.98 (d, J = 19.85 Hz, 2H), 3.69 (t, J = 7.20 Hz, 2H), 2.85 (t, J = 7.20 Hz, 2H), 2.52 (s, 3H), 2.11 (t, J = 7.01 Hz, 2H); LCMS: ESI [M + H]+ = 453.1 |
| 397 | 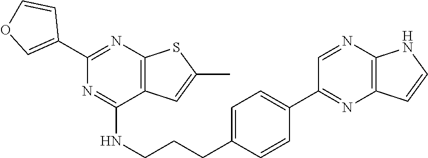 [SMILES: Cc1cc2c(NCCCc3ccc(-c4cnc5[nH]ccc5n4)cc3)nc(-c3ccoc3)nc2s1] | KH 6067 | 85% | Route A Cs₂CO₃ 3 h (90° C.) 9 h (100° C.) | 9% | 2-(furan-3-yl)-6- methyl-N-[3-(4- (5H-pyrrolo[2,3- b]pyrazin-2- yl)phenyl)propyl] thieno[2,3-d] pyrimidin-4- amine | ¹H NMR (399 MHz, METHANOL-d4) δ 8.64 (s, 1H), 8.06 (s, 1H), 7.88 (d, J = 8.17 Hz, 2H), 7.76 (d, J = 3.50 Hz, 1H), 7.47 (s, 1H), 7.37 (d, J = 7.79 Hz, 2H), 7.00 (s, 1H), 6.94 (s, 1H), 6.66 (d, J = 3.50 Hz, 1H), 3.68 (t, J = 7.20 Hz, 2H), 2.81 (t, J = 7.20 Hz, 2H), 2.49 (s, 3H), 2.09 (t, J = 7.01 Hz, 2H); LCMS: ESI [M + H]+ = 467.2 |
| 398 | 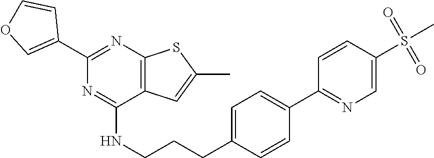 [SMILES: Cc1cc2c(NCCCc3ccc(-c4ccc(S(C)(=O)=O)cn4)cc3)nc(-c3ccoc3)nc2s1] | KH 6067 | 85% | Route A Cs₂CO₃ 2 h (90° C.) | 32% | 2-(furan-3-yl)-N- (3-[4-(5- methanesulfonyl- pyridin-2-yl) phenyl]propyl)- 6-methylthieno [2,3-d]pyrimidin- 4-amine | ¹H NMR (399 MHz, METHANOL-d4) δ 9.09 (s, 1H), 8.33 (d, J = 8.56 Hz, 1H), 8.00-8.08 (m, 4H), 7.48 (s, 1H), 7.38 (d, J = 8.17 Hz, 2H), 7.00 (s, 1H), 6.93 (s, 1H), 3.68 (t, J = 6.81 Hz, 2H), 3.22 (s, 3H), 2.83 (t, J = 7.20 Hz, 2H), 2.50 (s, 3H), 2.03-2.18 (m, 2H); LCMS: ESI [M + H]+ = 505.1 |
| 399 | 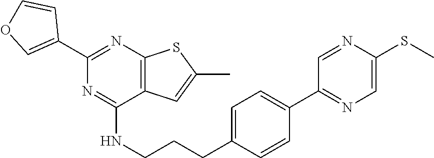 [SMILES: CSc1cnc(-c2ccc(CCCNc3nc(-c4ccoc4)nc4sc(C)cc34)cc2)cn1] | KH 6067 | 85% | Route A Cs₂CO₃ 2 h (90° C.) | 38% | 2-(furan-3-yl)-6- methyl-N-(3-(4- [5- (methylsulfanyl) pyrazin-2-yl] phenyl)propyl) thieno[2,3-d] pyrimidin-4- amine | ¹H NMR (399 MHz, ACETONITRILE-d3) δ 8.86 (s, 1H), 8.51 (s, 1H), 8.10 (s, 1H), 7.93 (dd, J = 1.95, 8.17 Hz, 2H), 7.50 (s, 1H), 7.36 (d, J = 6.23 Hz, 2H), 6.93 (d, J = 12.07 Hz, 2H), 6.15 (br. s., 1H), 3.65 (q, J = 6.23 Hz, 2H), 2.79 (t, J = 7.20 Hz, 2H), 2.60 (s, 3H), 2.51 (s, 3H), 2.00-2.07 (m, 2H); LCMS: ESI [M + H]+ = 474.1 |
| 400 | 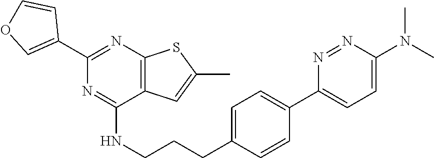 [SMILES: Cc1cc2c(NCCCc3ccc(-c4ccc(N(C)C)nn4)cc3)nc(-c3ccoc3)nc2s1] | KH 6067 | 85% | Route A K₂CO₃ 3 h (100° C.) | 26% | 6-[4-(3-([2- (furan-3-yl)-6- methylthieno [2,3-d]pyrimidin- 4- yl]amino)propyl) phenyl]-N,N- dimethylpyridazin- 3-amine | ¹H NMR (399 MHz, METHANOL-d4) δ 8.05 (s, 1H), 7.72-7.84 (m, 3H), 7.48 (d, J = 1.56 Hz, 1H), 7.32 (d, J = 7.79 Hz, 2H), 7.15 (d, J = 9.34 Hz, 1H), 6.99 (s, 1H), 6.93 (s, 1H), 3.66 (t, J = 7.01 Hz, 2H), 3.18 (s, 6H), 2.78 (t, J = 7.40 Hz, 2H), 2.50 (s, 3H), 1.99-2.14 (m, 2H); LCMS: ESI [M + H]+ = 471.2 |
| 401 | 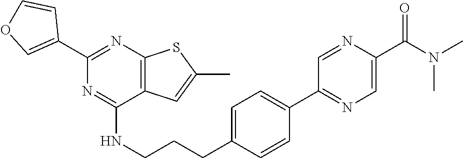 [SMILES: Cc1cc2c(NCCCc3ccc(-c4cnc(C(=O)N(C)C)cn4)cc3)nc(-c3ccoc3)nc2s1] | KH 6067 | 85% | Route A Cs₂CO₃ 2 h (90° C.) | 60% | 5-[4-(3-([2- (furan-3-yl)-6- methylthieno[2,3- d]pyrimidin-4- yl]amino)propyl) phenyl]-N,N- dimethylpyrazine- 2-carboxamide | ¹H NMR (399 MHz, METHANOL-d4) δ 9.02 (s, 1H), 8.83 (s, 1H), 7.93-8.03 (m, 3H), 7.46 (s, 1H), 7.34 (d, J = 7.01 Hz, 2H), 6.94 (d, J = 15.57 Hz, 2H), 3.60-3.68 (m, 2H), 3.14 (s, 6H), 2.74-2.83 (m, 2H), 2.47 (s, 3H), 1.97-2.12 (m, 2H); LCMS: ESI [M + H]+ = 499.2 |
| 402 | 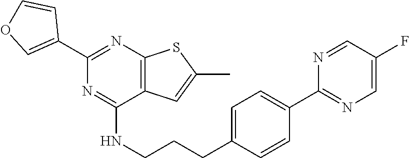 [SMILES: Cc1cc2c(NCCCc3ccc(-c4ncc(F)cn4)cc3)nc(-c3ccoc3)nc2s1] | KH 6067 | 85% | Route A Cs₂CO₃ 1.5 h (90° C.) | 54% | N-(3-[4-(5- fluoropyrimidin- 2-yl)phenyl] propyl)-2-(furan- 3-yl)-6- methylthieno[2,3- d]pyrimidin-4- amine | ¹H NMR (399 MHz, ACETONITRILE-d3) δ 8.71 (s, 2H), 8.27 (d, J = 7.79 Hz, 2H), 8.10 (s, 1H), 7.49 (s, 1H), 7.37 (d, J = 7.79 Hz, 2H), 6.92 (d, J = 7.40 Hz, 2H), 6.18 (br. s., 1H), 3.65 (q, J = 6.49 Hz, 2H), 2.80 (t, J = 7.40 Hz, 2H), 2.51 (s, 3H), 2.03 (t, J = 7.20 Hz, 2H); LCMS: ESI [M + H]+ = 446.1 |
| 403 | 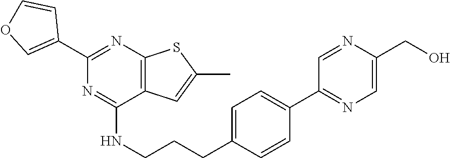 [SMILES: Cc1cc2c(NCCCc3ccc(-c4cnc(CO)cn4)cc3)nc(-c3ccoc3)nc2s1] | KH 6067 | 85% | Route A Cs₂CO₃ 2 h (90° C.) | 13% | (5-[4-(3-([2- (furan-3-yl)-6- methylthieno[2,3- d]pyrimidin-4- yl]amino)propyl) phenyl]pyrazin- 2-yl)methanol | ¹H NMR (399 MHz, METHANOL-d4) δ 8.95 (s, 1H), 8.72 (s, 1H), 8.03 (s, 1H), 7.93 (d, J = 7.79 Hz, 2H), 7.46 (s, 1H), 7.36 (d, J = 7.79 Hz, 2H), 6.98 (s, 1H), 6.92 (s, 1H), 4.77 (s, 2H), 3.66 (t, J = 6.81 Hz, 2H), 2.80 (t, J = 7.20 Hz, 2H), 2.49 (s, 3H), 2.07 (t, J = 7.20 Hz, 2H); LCMS: ESI [M + H]+ = 458.2 |
| 404 | 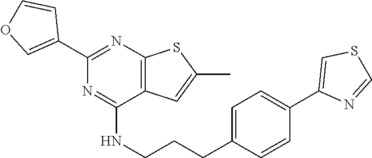 [SMILES: Cc1cc2c(NCCCc3ccc(-c4cscn4)cc3)nc(-c3ccoc3)nc2s1] | KH 6067 | 85% | Route A K₂CO₃ 3 h (100° C.) | 31% | 2-(furan-3-yl)-6- methyl-N-(3-[4- (1,3-thiazol- 4-yl)phenyl] propyl)thieno [2,3-d]pyrimidin- 4-amine | 1H NMR (399 MHz, DMSO-d6) δ 9.14 (d, J = 1.56 Hz, 1H), 8.16 (s, 1H), 8.07 (d, J = 1.95 Hz, 1H), 7.88 (d, J = 8.17 Hz, 2H), 7.77 (t, J = 5.45 Hz, 1H), 7.63 (s, 1H), 7.29 (d, J = 8.17 Hz, 2H), 7.20 (s, 1H), 6.86 (s, 1H), 3.53 (q, J = 6.62 Hz, 2H), 2.69 (t, J = 7.40 Hz, 2H), 2.50 (s, 3H), 1.93 (t, J = 7.01 Hz, 2H); LCMS: ESI [M + H]+ = 433.1 |
| 405 | 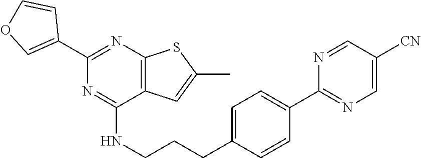 [SMILES: Cc1cc2c(NCCCc3ccc(-c4ncc(C#N)cn4)cc3)nc(-c3ccoc3)nc2s1] | KH 6067 | 85% | Route A K₂CO₃ 2 h (90° C.) | 10% | 2-[4-(3-([2-(furan- 3-yl)-6- methylthieno[2,3- d]pyrimidin-4- yl]amino)propyl) phenyl] pyrimidine-5- carbonitrile | ¹H NMR (399 MHz, ACETONITRILE-d3) δ 9.08 (s, 2H), 8.37 (d, J = 8.17 Hz, 2H), 8.07 (s, 1H), 7.47 (s, 1H), 7.40 (d, J = 8.17 Hz, 2H), 6.87-6.93 (m, 2H), 6.15 (br. s., 1H), 3.64 (q, J = 6.49 Hz, 2H), 2.82 (t, J = 7.40 Hz, 2H), 2.49 (s, 3H), 2.01-2.06 (m, 2H); LCMS: ESI [M + H]+ = 453.1 |
| 406 | 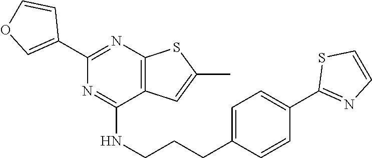 [SMILES: Cc1cc2c(NCCCc3ccc(-c4nccs4)cc3)nc(-c3ccoc3)nc2s1] | KH 6067 | 85% | Route A K₂CO₃ 2 h (90° C.) | 16% | 2-(furan-3-yl)-6- methyl-N-(3-[4- (1,3-thiazol-2- yl)phenyl]propyl) thieno[2,3- d]pyrimidin-4- amine | ¹H NMR (399 MHz, CHLOROFORM-d) δ 8.16 (s, 1H), 7.82-7.96 (m, 3H), 7.44 (s, 1H), 7.29-7.36 (m, 3H), 7.02 (s, 1H), 6.56 (s, 1H), 4.88 (br. s., 1H), 3.74 (q, J = 6.36 Hz, 2H), 2.83 (t, J = 7.20 Hz, 2H), 2.47 (s, 3H), 2.10 (quin, J = 7.01 Hz, 2H); LCMS: ESI [M + H]+ = 433.1 |
| 407 | 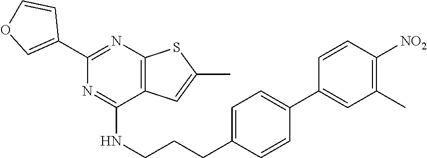 [SMILES: Cc1cc2c(NCCCc3ccc(-c4ccc([N+](=O)[O-])c(C)c4)cc3)nc(-c3ccoc3)nc2s1] | KH 6067 | 85% | Route A Cs₂CO₃ 2 h (80° C.) | 47% | 2-(furan-3-yl)-6- methyl-N-(3-(3′- methyl-4′-nitro- [1,1′-biphenyl]-4- yl)propyl)thieno [2,3-d]pyrimidin- 4-amine | ¹H NMR (399 MHz, METHANOL-d4) δ 7.99-8.08 (m, 2H), 7.55-7.67 (m, 4H), 7.48 (s, 1H), 7.33 (d, J = 7.40 Hz, 2H), 6.88-7.04 (m, 2H), 3.67 (t, J = 7.01 Hz, 2H), 2.80 (t, J = 7.20 Hz, 2H), 2.63 (s, 3H), 2.50 (s, 3H), 2.07 (quin, J = 6.91 Hz, 2H); LCMS: ESI [M + H]+ = 485.1 |
| 408 | 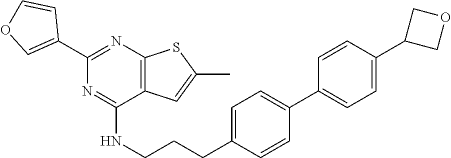 [SMILES: Cc1cc2c(NCCCc3ccc(-c4ccc(C5COC5)cc4)cc3)nc(-c3ccoc3)nc2s1] | KH 6067 | 85% | Route A Cs₂CO₃ 2 h (80° C.) | 38% | 2-(furan-3-yl)-6- methyl-N-(3-[4′- (oxetan-3-yl)- [1,1′-biphenyl]-4- yl]propyl)thieno [2,3-d]pyrimidin- 4-amine | ¹H NMR (399 MHz, ACETONITRILE-d3) δ 8.09 (s, 1H), 7.63 (d, J = 7.79 Hz, 2H), 7.56 (d, J = 7.40 Hz, 2H), 7.45-7.51 (m, 3H), 7.33 (d, J = 7.79 Hz, 2H), 6.93 (d, J = 7.40 Hz, 2H), 6.16 (br. s., 1H), 5.01 (dd, J = 6.62, 7.79 Hz, 2H), 4.70 (t, J = 6.42 Hz, 2H), 4.29 (quin, J = 7.59 Hz, 1H), 3.65 (q, J = 6.36 Hz, 2H), 2.78 (t, J = 7.40 Hz, 2H), 2.51 (s, 3H), 2.04 (d, J = 7.01 Hz, 2H); LCMS: ESI [M + H]+ = 482.2 |
| 409 | 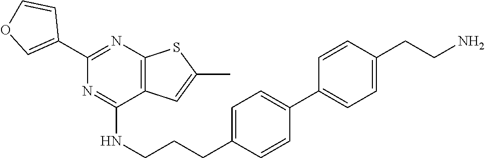 [SMILES: Cc1cc2c(NCCCc3ccc(-c4ccc(CCN)cc4)cc3)nc(-c3ccoc3)nc2s1] | KH 6067 | 85% | Route A Cs₂CO₃ 2 h (80° C.) | 20% | N-(3-[4′-(2- aminoethyl)- [1,1′-biphenyl]-4- yl]propyl)-2- (furan-3-yl)-6- methylthieno[2,3- d]pyrimidin-4- amine | ¹H NMR (399 MHz, METHANOL-d4) δ 8.05 (s, 1H), 7.42-7.56 (m, 5H), 7.26 (d, J = 7.79 Hz, 4H), 6.88-7.02 (m, 2H), 3.65 (t, J = 7.01 Hz, 2H), 2.86-2.94 (m, 2H), 2.69-2.83 (m, 4H), 2.49 (s, 3H), 2.04 (t, J = 7.01 Hz, 2H); LCMS: ESI [M + H]+ = 469.2 |
| 410 | 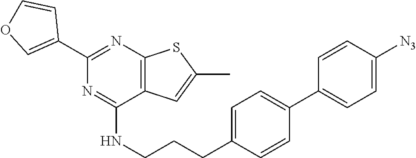 [SMILES: Cc1cc2c(NCCCc3ccc(-c4ccc(N=[N+]=[N-])cc4)cc3)nc(-c3ccoc3)nc2s1] | KH 6067 | 85% | Route A Cs₂CO₃ 2 h (90° C.) | 24% | N-(3-(4′-azido- [1,1′-biphenyl]-4- yl)propyl)-2- (furan-3-yl)-6- methylthieno[2,3- d]pyrimidin-4- amine | ¹H NMR (399 MHz, ACETONITRILE-d3) δ 8.09 (s, 1H), 7.64 (d, J = 8.17 Hz, 2H), 7.48-7.57 (m, 3H), 7.32 (d, J = 8.17 Hz, 2H), 7.15 (d, J = 8.17 Hz, 2H), 6.93 (d, J = 8.17 Hz, 2H), 6.16 (br. s., 1H), 3.65 (q, J = 6.62 Hz, 2H), 2.78 (t, J = 7.59 Hz, 2H), 2.52 (s, 3H), 2.02 (t, J = 7.20 Hz, 2H); LCMS: ESI [M + H]+ = 467.1 |
| 411 | 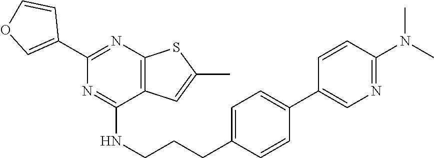 [SMILES: Cc1cc2c(NCCCc3ccc(-c4ccc(N(C)C)nc4)cc3)nc(-c3ccoc3)nc2s1] | KH 6067 | 85% | Route A Cs₂CO₃ 2 h (90° C.) | 12% | 5-[4-(3-([2- (furan-3-yl)-6- methylthieno[2,3- d]pyrimidin-4- yl]amino)propyl) phenyl]-N,N- dimethylpyridin- 2-amine | ¹H NMR (399 MHz, ACETONITRILE-d3) δ 8.38 (s, 1H), 8.09 (s, 1H), 7.75 (d, J = 8.95 Hz, 1H), 7.49 (d, J = 8.56 Hz, 3H), 7.29 (d, J = 7.79 Hz, 2H), 6.93 (d, J = 8.56 Hz, 2H), 6.66 (d, J = 8.95 Hz, 1H), 6.16 (br. s., 1H), 3.64 (; LCMS: ESI [M + H]+ = q, J = 6.23 Hz, 2H), 3.08 (s, 6H), 2.75 (t, J = 7.59 Hz, 2H), 2.52 (s, 3H), 1.98-2.07 (m, 2H); LCMS: ESI [M + H]+ = 470.2 |
| 412 | 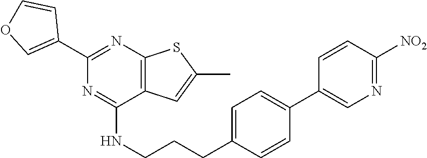 [SMILES: Cc1cc2c(NCCCc3ccc(-c4ccc([N+](=O)[O-])nc4)cc3)nc(-c3ccoc3)nc2s1] | KH 6067 | 85% | Route A K₂CO₃ 2 h (90° C.) | 39% | 2-(furan-3-yl)-6- methyl-N-(3-[4- (6-nitropyridin-3- yl)phenyl]propyl) thieno[2,3- d]pyrimidin-4- amine | 1H NMR (399 MHz, ACETONITRILE-d3) δ 8.85 (s, 1H), 8.30 (s, 2H), 8.07 (s, 1H), 7.66 (d, J = 7.79 Hz, 2H), 7.50 (d, J = 1.17 Hz, 1H), 7.42 (d, J = 7.79 Hz, 2H), 6.93 (d, J = 9.34 Hz, 2H), 6.17 (br. s., 1H), 3.66 (q, J = 6.62 Hz, 2H), 2.82 (t, J = 7.40 Hz, 2H), 2.51 (s, 3H), 2.05 (t, J = 7.20 Hz, 2H); LCMS: ESI [M + H]+ = 472.1 |
| 413 | 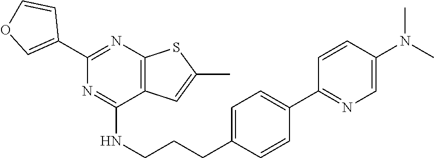 [SMILES: Cc1cc2c(NCCCc3ccc(-c4ccc(N(C)C)cn4)cc3)nc(-c3ccoc3)nc2s1] | KH 6067 | 85% | Route A Cs₂CO₃ 5 h (90° C.) | 25% | 6-[4-(3-([2-(furan- 3-yl)-6- methylthieno[2,3- d]pyrimidin-4- yl]amino)propyl) phenyl]-N,N- dimethyl-pyridin- 3-amine | ¹H NMR (399 MHz, ACETONITRILE-d3) δ 8.18 (d, J = 2.72 Hz, 1H), 8.10 (s, 1H), 7.87 (d, J = 8.17 Hz, 2H), 7.65 (d, J = 8.95 \|Hz, 1H), 7.49 (s, 1H), 7.29 (d, J = 7.79 Hz, 2H), 7.13 (dd, J = 3.11, 8.95 Hz, 1H), 6.92 (d, J = 8.17 Hz, 2H), 6.15 (br. s., 1H), 3.64 (q, J = 6.62 Hz, 2H), 2.99 (s, 6H), 2.76 (t, J = 7.40 Hz, 2H), 2.50 (s, 3H), 2.01 (t, J = 7.20 Hz, 2H); LCMS: ESI [M + H]+ = 470.2 |
| 414 | 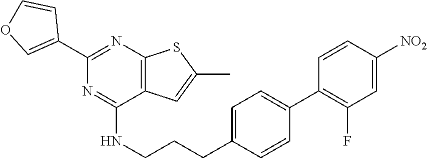 [SMILES: Cc1cc2c(NCCCc3ccc(-c4ccc([N+](=O)[O-])cc4F)cc3)nc(-c3ccoc3)nc2s1] | KH 6067 | 85% | Route A K₂CO₃ 2 h (100° C.) | 36% | N-(3-(2′-fluoro- 4′-nitro-[1,1′- biphenyl]-4-yl) propyl)-2-(furan- 3-yl)-6- methylthieno[2,3- d]pyrimidin-4- amine | ¹H NMR (399 MHz, ACETONITRILE-d3) δ 8.04-8.16 (m, 3H), 7.71 (t, J = 8.17 Hz, 1H), 7.48-7.57 (m, 3H), 7.40 (d, J = 7.79 Hz, 2H), 6.94 (br. s., 2H), 6.20 (br. s., 1H), 3.66 (q, J = 6.62 Hz, 2H), 2.82 (t, J = 7.40 Hz, 2H), 2.53 (s, 3H), 2.05 (br. s., 2H); LCMS: ESI [M + H]+ = 489.1 |
| 415 | 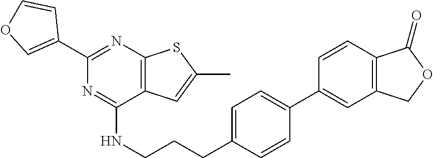 [SMILES: Cc1cc2c(NCCCc3ccc(-c4ccc5c(c4)COC5=O)cc3)nc(-c3ccoc3)nc2s1] | KH 6067 | 85% | Route A K₂CO₃ 2 h (100° C.) | 17% | 5-[4-(3-([2- (furan-3-yl)-6- methylthieno[2,3- d]pyrimidin-4- yl]amino)propyl) phenyl]-1,3- dihydro-2- benzofuran-1-one | ¹H NMR (399 MHz, METHANOL-d4) δ 8.03 (s, 1H), 7.87-7.93 (m, 1H), 7.75- 7.84 (m, 2H), 7.60 (d, J = 7.79 Hz, 2H), 7.48 (s, 1H), 7.35 (d, J = 7.79 Hz, 2H), 7.00 (s, 1H), 6.93 (s, 1H), 5.42 (s, 2H), 3.67 (t, J = 7.59 Hz, 2H), 2.80 (t, J = 7.01 Hz, 2H), 2.50 (s, 3H), 2.08 (t, J = 7.20 Hz, 2H); LCMS: ESI [M + H]+ = 482.1 |
| 416 | 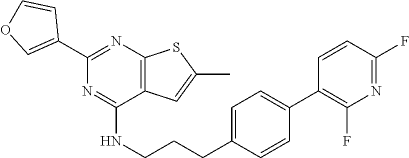 [SMILES: Cc1cc2c(NCCCc3ccc(-c4ccc(F)nc4F)cc3)nc(-c3ccoc3)nc2s1] | KH 6067 | 85% | Route A K₂CO₃ 2 h (100° C.) | 43% | N-(3-[4-(2,6- difluoro-pyridin- 3-yl)phenyl] propyl)-2-(furan- 3-yl)-6- methylthieno[2,3- d]pyrimidin-4- amine | ¹H NMR (399 MHz, ACETONITRILE-d3) δ 8.03-8.16 (m, 2H), 7.43-7.53 (m, 3H), 7.36 (d, J = 7.79 Hz, 2H), 7.04 (dd, J = 2.34, 8.17 Hz, 1H), 6.94 (d, J = 8.56 Hz, 2H), 6.17 (br. s., 1H), 3.64 (q, J = 6.62 Hz, 2H), 2.78 (t, J = 7.40 Hz, 2H), 2.52 (s, 3H), 2.02 (t, J = 7.20 Hz, 2H); LCMS: ESI [M + H]+ = 463.0 |
| 417 | 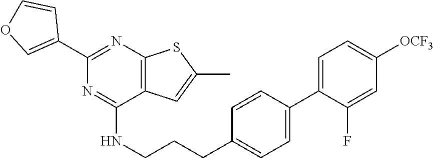 [SMILES: Cc1cc2c(NCCCc3ccc(-c4ccc(OC(F)(F)F)cc4F)cc3)nc(-c3ccoc3)nc2s1] | KH 6067 | 85% | Route A K₂CO₃ 1.5 h (100° C.) | 47% | N-(3-[2′-fluoro- 4′-(trifluoro- methoxy)-[1,1′- biphenyl]-4- yl]propyl)-2- (furan-3-yl)-6- methylthieno[2,3- d]pyrimidin-4- amine | ¹H NMR (399 MHz, CHLOROFORM-d) δ 8.15 (s, 1H), 7.42-7.51 (m, 4H), 7.33 (d, J = 7.40 Hz, 2H), 7.01-7.13 (m, 3H), 6.61 (s, 1H), 4.95 (br. s., 1H), 3.75 (q, J = 6.23 Hz, 2H), 2.83 (t, J = 7.20 Hz, 2H), 2.51 (s, 3H), 2.11 (t, J = 7.01 Hz, 2H); LCMS: ESI [M + H]+ = 528.1 |
| 418 | 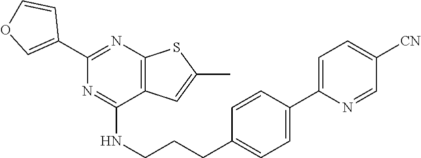 [SMILES: Cc1cc2c(NCCCc3ccc(-c4ccc(C#N)cn4)cc3)nc(-c3ccoc3)nc2s1] | KH 6067 | 85% | Route A K₂CO₃ 2 h (100° C.) | 51% | 6-[4-(3-([2- (furan-3-yl)-6- methylthieno[2,3- d]pyrimidin-4- yl]amino)propyl) phenyl]pyridine- 3-carbonitrile | ¹H NMR (399 MHz, CHLOROFORM-d) δ 8.94 (br. s., 1H), 8.12 (s, 1H), 7.99 (d, J = 7.79 Hz, 3H), 7.82 (d, J = 8.17 Hz, 1H), 7.44 (s, 1H), 7.37 (d, J = 7.79 Hz, 2H), 7.03 (s, 1H), 6.61 (s, 1H), 4.97 (br. s., 1H), 3.73 (d, J = 6.23 Hz, 2H), 2.76- 2.90 (m, 2H), 2.49 (s, 3H), 2.10 (t, J = 7.01 Hz, 2H); LCMS: ESI [M + H]+ = 452.1 |
| 419 | 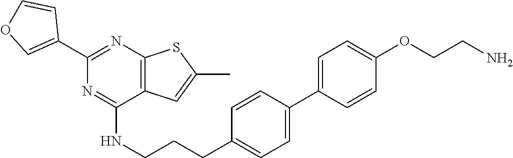 [SMILES: Cc1cc2c(NCCCc3ccc(-c4ccc(OCCN)cc4)cc3)nc(-c3ccoc3)nc2s1] | KH 6067 | 85% | Route A K₂CO₃ 2 h (100° C.) | 10% | N-(3-[4′-(2- aminoethoxy)- [1,1′-biphenyl]-4- yl]propyl)-2- (furan-3-yl)-6- methylthieno[2,3- d]pyrimidin-4- amine | ¹H NMR (399 MHz, METHANOL-d4) δ 8.04 (s, 1H), 7.42-7.53 (m, 5H), 7.24 (d, J = 7.79 Hz, 2H), 6.97-7.02 (m, 3H), 6.91 (s, 1H), 4.04 (t, J = 5.06 Hz, 2H), 3.65 (t, J = 7.01 Hz, 2H), 3.03 (t, J = 5.26 Hz, 2H), 2.75 (t, J = 7.01 Hz, 2H), 2.50 (s, 3H), 1.96-2.10 (m, 2H); LCMS: ESI [M + H]+ = 485.2 |
| 420 | | KH 4007 | 67% | 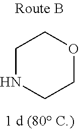 | 37% | 2-ethyl-6-methyl- N-(3-(4- morpholinophenyl) propyl)thieno[2,3- d]pyrimidin-4- amine | 1H NMR (400 MHz, CHLOROFORM-d) δ 7.21-7.10 (m, 2H), 6.90-6.82 (m, 2H), 6.57-6.52 (m, 1H), 3.90-3.83 (m, 4H), 3.65 (q, J = 6.5 Hz, 2H), 3.16-3.09 (m, 4H), 2.82 (q, J = 7.6 Hz, 2H), 2.69 (t, J = 7.3 Hz, 2H), 2.52 (d, J = 1.2 Hz, 3H), 1.99 (quin, J = 7.1 Hz, 2H), 1.34 (t, J = 7.6 Hz, 3H); LCMS: ESI [M + H]⁺ = 397.2 |
| 421 | | KH 4007 | 67% | 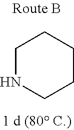 | 18% | 2-ethyl-6-methyl- N-(3-(4- (piperidin-1- yl)phenyl)propyl) thieno[2,3- d]pyrimidin- 4-amine | ¹H NMR (400 MHz, METHANOL-d₄) δ 6.96-7.07 (m, 2H), 6.91 (d, J = 10.17 Hz, 1H), 6.77-6.85 (m, 2H), 3.49 (td, J = 7.09, 10.86 Hz, 2H), 2.97 (td, J = 5.67, 10.56 Hz, 4H), 2.52-2.69 (m, 4H), 2.44 (d, J = 10.17 Hz, 3H), 1.79-1.97 (m, 2H), 1.56- 1.70 (m, 4H), 1.42-1.53 (m, 2H), 1.21 (td, J = 7.63, 11.35 Hz, 3H); LCMS: ESI |
| | | | | | | | [M + H]⁺ = 395.2 |
| 422 | 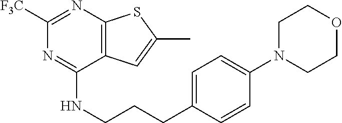 [SMILES: Cc1cc2c(NCCCc3ccc(N4CCOCC4)cc3)nc(C(F)(F)F)nc2s1] | KH 6080 | 65% | 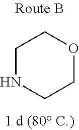 | 18% | 6-methyl-N-(3-(4- morpholinophenyl) propyl)-2- (trifluoromethyl) thieno[2,3- d]pyrimidin-4- amine | ¹H NMR (400 MHz, CHLOROFORM-d) δ 7.16 (d, J = 8.22 Hz, 2H), 6.90 (br. s., 2H), 6.61 (s, 1H), 5.11 (br. s., 1H), 3.88 (br. s., 4H), 3.70 (q, J = 6.39 Hz, 2H), 3.16 (d, J = 3.91 Hz, 4H), 2.71 (t, J = 7.24 Hz, 2H), 2.59 (s, 3H), 1.95-2.10 (m, 2H); LCMS: ESI [M + H]⁺ = 437.2 |
| 423 | 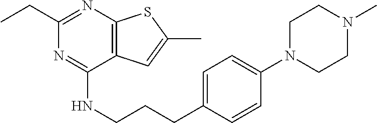 [SMILES: CCc1nc(NCCCc2ccc(N3CCN(C)CC3)cc2)c2cc(C)sc2n1] | KH 4007 | 67% | 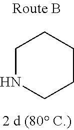 | 10% | 2-ethyl-6-methyl- N-(3-(4-(4- methylpiperazin-1- yl)phenyl)propyl) thieno[2,3- d]pyrimidin-4- amine | ¹H NMR (400 MHz, CHLOROFORM-d) δ 7.10 (d, J = 8.61 Hz, 2H), 7.00 (d, J = 1.17 Hz, 1H), 6.89 (d, J = 8.61 Hz, 2H), 3.58 (t, J = 7.04 Hz, 2H), 3.13 (d, J = 4.70 Hz, 4H), 2.72 (q, J = 7.43 Hz, 2H), 2.60-2.66 (m, 6H), 2.53 (d, J = 1.17 Hz, 3H), 2.35 (s, 3H), 1.97 (quin, J = 7.24 Hz, 2H), 1.30 (t, J = 7.43 Hz, 4H); LCMS: ESI [M + H]⁺ = 410.3 |

### Example 6: Scheme 4 Biaryl-Propylamine Synthesis+S_{N}Ar

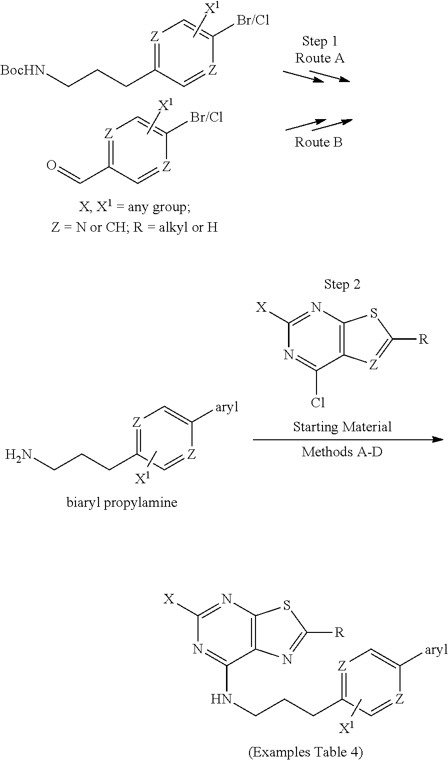

General Procedure for Scheme 4: Reaction conditions and compound data for specific examples are listed in Table 4. Starting materials and reagents for step 1 were commercially purchased, the step 2 starting materials were synthesized as detailed in the building blocks section, unless otherwise stated.

Step 1 (Route A): Following the General Suzuki Protocol with Cs₂CO₃, the appropriate Boc-protected bromophenyl amine (0.030 mmol) and the desired aryl boronic acid (0.060 mmol) were reacted for 2 hours at 80° C. Upon completion, the reaction was cooled to room temperature and the solvents were evaporated under reduced pressure and the crude reaction residue was purified by silica gel column chromatography. The product was then dissolved in 10% trifluoroacetic acid (TFA) in DCM and stirred overnight at room temperature. Upon completion, the solvents were evaporated to give the desired biphenyl amine derivative (as a TFA salt), for direct use in step 2.

### Step 1 (Route B):

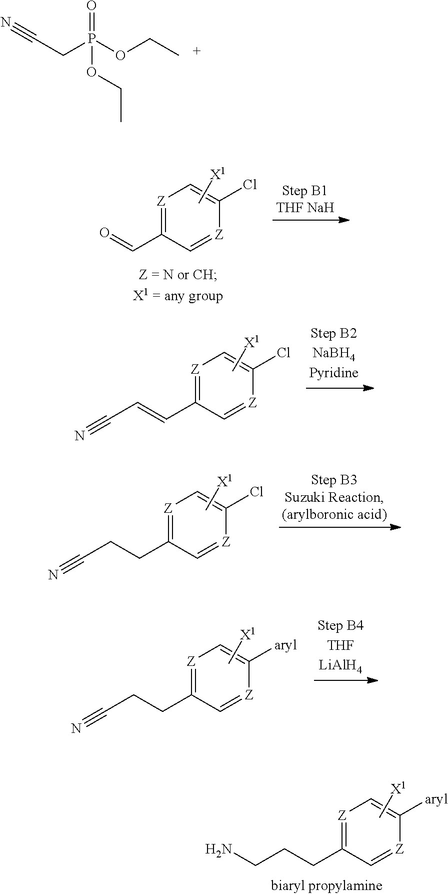

Step B1: Diethyl cyanomethylphosphonate (2.111 mmol) was dissolved in anhydrous THF (6.0 mL) and vial was attached to a bubbler. NaH (2.111 mmol) was carefully added and reaction was rapidly stirred at room temperature for 10 minutes. The reaction was cooled on an ice bath before adding the appropriate substituted 4-chlorobenzaldehyde (1.759 mmol). The reaction was allowed to slowly warm to room temperature and was complete within an hour. The reaction was then concentrated in vacuo and purified by silica gel column chromatography to give the intermediate (4-chlorophenyl)acrylonitrile derivative for use in step B2.

Step B2: The (4-chlorophenyl)acrylonitrile intermediate (1.210 mmol) was dissolved in pyridine (1.95 mL) and anhydrous MeOH (0.65 mL). Sodium borohydride (1.820 mmol) was added and reacted for 16 hours at 60° C. while attached to a bubbler. Upon completion, the reaction was concentrated in vacuo and the product was extracted into DCM (3×) from saturated NH₄Cl(aq). The organic layer was dried over Na₂SO₄ and concentrated in vacuo to give the 3-(4-chlorophenyl)propanenitrile intermediate for use in step B3.

Step B3: Following the General Suzuki Protocol, using Cs₂CO₃, the 3-(4-chlorophenyl)propanenitrile intermediate (0.525 mmol) and the desired arylboronic acid (0.525 mmol) were reacted in a microwave reactor for 1 hour at 150° C. The reaction was purified by silica gel column chromatography to give the pure biaryl-propanenitrile product for use in step B4.

Step B4: The biaryl-propanenitrile intermediate (0.352 mmol) was dissolved in anhydrous THF (2.0 mL) and vial was cooled on an ice bath before attaching to a bubbler. A solution of 2.5M lithium aluminum hydride in THF (0.387 mmol) was carefully added and reacted for 1 hour at 0° C. Saturated NH₄Cl(aq) was slowly added to neutralize the reaction, then MeOH (15 mL) was added to crash out byproducts. Precipitates were filtered out and the solvent was concentrated in vacuo. Product was dissolved in MeOH, filtered and purified by prep HPLC to give the desired biaryl propylamine (yield range over steps B1-B4; 1-6%)

Step 2: The biaryl propylamine (from step 1. Route A or B) and the appropriate starting material, were reacted via one of the procedures described in Scheme 1, Methods A-D, as specified, and purified by prep HPLC or silica gel column chromatography, or a combination of the two, to give the desired product (Table 4).

TABLE 4
Examples from Scheme 4

| | | | Step | Step 2 | Step | | |
|---|---|---|---|---|---|---|---|
| Cmpd. | | | 1 | Method/ | 2 | | |
| No. | Structure | Starting Material | Route | Conditions | Yield | IUPAC | Compound data |
| 424 | 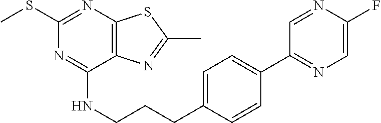 [SMILES: CSc1nc(NCCCc2ccc(-c3cnc(F)cn3)cc2)c2nc(C)sc2n1] | LM 13029 | A | Method A 4 h (50° C.) | 54% | N-(3-[4-(5- fluoropyrazin-2- yl)phenyl]propyl)- 2-methyl-5- (methylsulfanyl)- [1,3]thiazolo[5,4- d]pyrimidin-7- amine | ¹H NMR (399 MHz, ACETONITRILE-d3) δ 8.67 (s, 1H), 8.52 (d, J = 8.17 Hz, 1H), 7.94 (d, J = 7.79 Hz, 2H), 7.39 (d, J = 7.79 Hz, 2H), 6.61 (br. s., 1H), 3.60 (q, J = 6.36 Hz, 2H), 2.78 (t, J = 7.59 Hz, 2H), 2.68 (s, 3H), 2.46 (s, 3H), |
| | | | | | | | 2.01-2.04 (m, 2H); LCMS: |
| | | | | | | | ESI [M + H]⁺ = 427.0 |
| 425 | 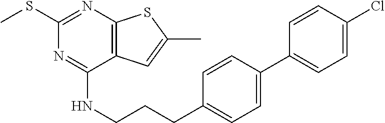 | KH 5002 | A | Method A 1 d (85° C.) | 53% | N-(3-(4′-chloro- [1,1′-biphenyl]-4- yl)propyl)-6- methyl-2- (methylthio)thieno [2,3-d]pyrimidin- 4-amine | ¹H NMR (400 MHz, CHLOROFORM-d) δ 7.42-7.48 (m, 4H), 7.36 (d, J = 8.31 Hz, 2H), 7.25 (d, J = 7.95 Hz, 2H), 6.83 (s, 1H), 3.62 (t, J = 7.03 Hz, 2H), 2.74 (t, J = 7.34 Hz, 2H), 2.45 (d, J = 2.32 Hz, 6H), 1.97-2.06 (m, 3H); LCMS: ESI |
| | | | | | | | [M + H]⁺ = 440.1 |
| 426 | 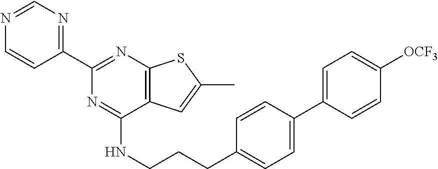 [SMILES: Cc1cc2c(NCCCc3ccc(-c4ccc(OC(F)(F)F)cc4)cc3)nc(-c3ccncn3)nc2s1] | KH 4057 | A | Method A 1 d (90° C.) | 47% | 6-methyl-2- (pyrimidin-4-yl)- N-(3-(4′- (trifluoromethoxy)- [1,1′-biphenyl]-4- yl)propyl)thieno [2,3-d]pyrimidin- 4-amine- | ¹H NMR (400 MHz, CHLOROFORM-d) δ 7.50 (dd, J = 6.54, 8.25 Hz, 4H), 7.40 (d, J = 8.56 Hz, 2H), 7.27-7.31 (m, 2H), 6.61 (s, 1H), 3.53-3.70 (m, 6H), 2.78 (t, J = 7.34 Hz, 2H), 2.32 (s, 3H), 2.06 (quin, J = 7.15 Hz, 2H), 1.88-2.00 (m, 4H); LCMS: ESI [M + H]⁺ = 522.2 |
| 427 | 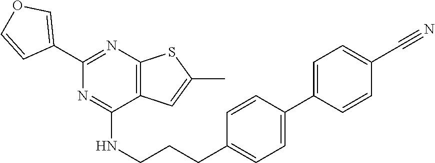 [SMILES: Cc1cc2c(NCCCc3ccc(-c4ccc(C#N)cc4)cc3)nc(-c3ccoc3)nc2s1] | KH 6067 | A | Method A 1 h (140° C.) microwave | 4% | 4′-(3-((2-(furan-3- yl)-6- methylthieno[2,3- d]pyrimidin-4- yl)amino)propyl)- [1,1′-biphenyl]-4- carbonitrile | ¹H NMR (400 MHz, METHANOL-d₄) δ 8.07 (s, 1H), 7.80 (s, 4H), 7.57-7.64 (m, J = 7.95 Hz, 2H), 7.51 (s, 1H), 7.34- 7.42 (m, J = 8.07 Hz, 2H), 7.03 (s, 1H), 6.97 (s, 1H), 3.71 (t, J = 6.97 Hz, 2H), 2.84 (t, J = 7.03 Hz, 2H), 2.54 (s, 3H), 2.11 (quin, J = 7.09 Hz, 2H); LCMS: ESI [M + H]⁺ = 451.2 |
| 428 | 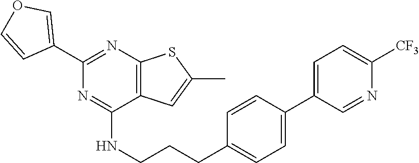 [SMILES: Cc1cc2c(NCCCc3ccc(-c4ccc(C(F)(F)F)nc4)cc3)nc(-c3ccoc3)nc2s1] | KH 6067 | A | Method A 1 h (140° C.) microwave | 3% | 2-(furan-3-yl)-6- methyl-N-(3-(4- (6- (trifluoromethyl) pyridin-3- yl)phenyl)propyl) thieno[2,3- d]pyrimidin-4- amine | ¹H NMR (400 MHz, METHANOL-d₄) δ 8.93 (s, 1H), 8.23 (d, J = 7.95 Hz, 1H), 8.04 (s, 1H), 7.87 (d, J = 8.19 Hz, 1H), 7.57-7.67 (m, J = 8.19 Hz, 2H), 7.50 (s, 1H), 7.35-7.45 (m, J = 8.07 Hz, 2H), 7.01 (s, 1H), 6.97 (s, 1H), 3.70 (t, J = 7.09 Hz, 2H), 2.84 (t, J = 7.40 Hz, 2H), 2.53 (s, 3H), 2.11 (quin, J = 7.15 |
| | | | | | | | Hz, 2H); LCMS: ESI |
| | | | | | | | [M + H]⁺ = 495.2 |
| 429 | 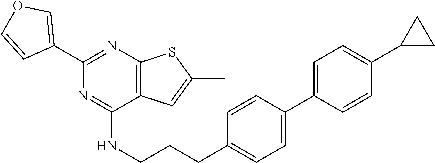 | KH 6067 | A | Method A 1 h (140° C.) microwave | 29% | N-(3-(4′- cyclopropyl-[1,1′- biphenyl]-4- yl)propyl)-2- (furan-3-yl)-6- methylthieno[2,3- d]pyrimidin-4- amine | ¹H NMR (400 MHz, CHLOROFORM-d) δ 8.05-8.40 (m, 1H), 7.41-7.59 (m, 5H), 7.29 (d, J = 8.07 Hz, 2H), 7.15 (d, J = 8.07 Hz, 3H), 6.36-6.81 (m, 1H), 3.67-3.84 (m, 2H), 2.84 (br. s., 2H), 2.44 (br. s., 3H), 2.13 (br. s., 2H), 1.87-2.01 (m, 1H), 0.94-1.07 (m, 2H), 0.70-0.80 (m, 2H); LCMS: ESI |
| | | | | | | | [M + H]⁺ = 466.2 |
| 430 | 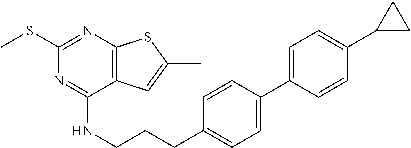 [SMILES: CSc1nc(NCCCc2ccc(-c3ccc(C4CC4)cc3)cc2)c2cc(C)sc2n1] | KH 5002 | A | Method A 3 d (85° C.) | 21% | N-(3-(4′- cyclopropyl-[1,1′- biphenyl]-4- yl)propyl)-6- methyl-2- (methylthio)thieno [2,3-d]pyrimidin-4- amine | ¹H NMR (400 MHz, CHLOROFORM-d) δ 7.47 (d, J = 8.19 Hz, 2H), 7.51 (d, J = 7.95 Hz, 2H), 7.23-7.27 (m, 2H), 7.14 (d, J = 8.07 Hz, 2H), 6.69 (br. s., 1H), 3.70 (q, J = 6.60 Hz, 2H), 2.79 (t, J = 7.21 Hz, 2H), 2.54 (s, 3H), 2.38 (s, 3H), 2.03-2.17 (m, 2H), 1.88- |
| | | | | | | | 2.00 (m, 1H), 0.94-1.07 (m, |
| | | | | | | | 2H), 0.68-0.80 (m, 2H); LCMS: |
| | | | | | | | ESI [M + H]⁺ = 446.2 |
| 431 | 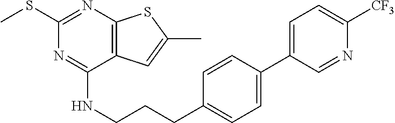 [SMILES: CSc1nc(NCCCc2ccc(-c3ccc(C(F)(F)F)nc3)cc2)c2cc(C)sc2n1] | KH 5002 | A | Method A 3 d (85° C.) | 2% | 6-methyl-2- (methylthio)-N-(3- (4-(6- (trifluoromethyl) pyridin-3-yl) phenyl)propyl) thieno[2,3- d]pyrimidin-4- amine | ¹H NMR (400 MHz, METHANOL-d₄) δ 8.93 (s, 1H), 8.52 (s, 1H), 7.87 (d, J = 8.31 Hz, 1H), 7.59-7.66 (m, J = 8.19 Hz, 2H), 7.36-7.42 (m, J = 8.07 Hz, 2H), 6.93 (s, 1H), 3.63 (t, J = 6.97 Hz, 2H), 2.81 (t, J = 7.52 Hz, 2H), 2.47 (d, J = 5.14 Hz, 6H), 2.07 (quin, J = 7.18 |
| | | | | | | | Hz, 2H); LCMS: ESI |
| | | | | | | | [M + H]⁺ = 475.2 |
| 432 | 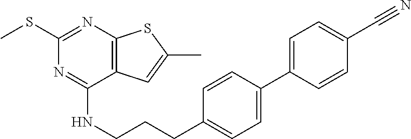 [SMILES: CSc1nc(NCCCc2ccc(-c3ccc(C#N)cc3)cc2)c2cc(C)sc2n1] | KH 5002 | A | Method A 3 d (85° C.) | 48% | 4′-(3-((6-methyl-2- (methylthio)thieno [2,3-d]pyrimidin- 4-yl)amino) propyl)-[1,1′- biphenyl]-4- carbonitrile | ¹H NMR (400 MHz, CHLOROFORM-d) δ 7.70-7.76 (m, J = 8.31 Hz, 2H), 7.63-7.69 (m, J = 8.44 Hz, 2H), 7.49-7.56 (m, J = 8.07 Hz, 2H), 7.30-7.35 (m, J = 7.95 Hz, 2H), 6.89 (br. s., 1H), 3.72 (q, J = 6.52 Hz, 2H), 2.82 (t, J = 7.34 Hz, 2H), 2.56 (s, 3H), 2.41 (s, 3H), 2.03- |
| | | | | | | | 2.18 (m, 2H); LCMS: ESI |
| | | | | | | | [M + H]⁺ = 431.2 |
| 433 | 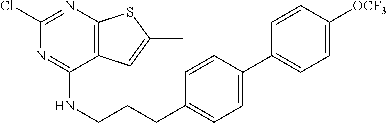 [SMILES: Cc1cc2c(NCCCc3ccc(-c4ccc(OC(F)(F)F)cc4)cc3)nc(Cl)nc2s1] | 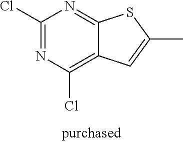 | A | Method A 1 d (40° C.) | 91% | 2-chloro-6-methyl- N-(3-(4′- (trifluoromethoxy)- [1,1′-biphenyl]-4- yl)propyl)thieno [2,3-d]pyrimidin- 4-amine | 1H NMR (400 MHz, CHLOROFORM-d) δ 7.60 (s, 2H), 7.51 (d, J = 8.2 Hz, 2H), 7.35-7.27 (m, 4H), 6.46 (d, J = 1.2 Hz, 1H), 4.98 (s, 1H), 3.69 (q, J = 6.8 Hz, 2H), 2.82 (t, J = 7.3 Hz, 2H), 2.41 (d, J = 1.2 Hz, 3H), 2.08 (quin, J = 6.9 Hz, 2H); LCMS: |
| | | | | | | | ESI [M + H]⁺ = 478.1 |
| 434 | 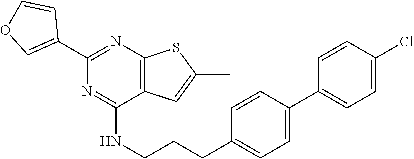 [SMILES: Cc1cc2c(NCCCc3ccc(-c4ccc(Cl)cc4)cc3)nc(-c3ccoc3)nc2s1] | KH 6067 | A | Method A 1 d (40° C.) | 54% | N-(3-(4′-chloro- [1,1′-biphenyl]-4- yl)propyl)-2- (furan-3-yl)-6- methylthieno[2,3- d]pyrimidin-4- amine | ¹H NMR (400 MHz, CHLOROFORM-d) δ 8.15 (br. s., 1H), 7.47-7.56 (m, 4H), 7.42 (t, J = 8.99 Hz, 3H), 7.31 (d, J = 8.07 Hz, 2H), 7.04 (br. s., 1H), 6.57 (br. s., 1H), 4.93 (br. s., 1H), 3.74 (q, J = 6.68 Hz, 2H), 2.83 (t, J = 7.15 Hz, 2H), 2.48 (s, 3H), 2.11 (quin, J = 6.94 Hz, 2H); LCMS: ESI [M + H]⁺ = 460.2 |
| 435 | 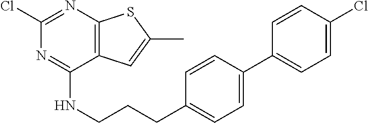 [SMILES: Cc1cc2c(NCCCc3ccc(-c4ccc(Cl)cc4)cc3)nc(Cl)nc2s1] | 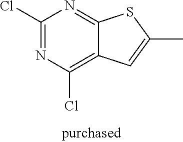 | A | Method A 1 d (45° C.) | 46% | 2-chloro-N-(3-(4′- chloro-[1,1′- biphenyl]-4- yl)propyl)-6- methylthieno[2,3- d]pyrimidin-4- amine | ¹H NMR (400 MHz, CHLOROFORM-d) δ 7.49-7.56 (m, 4H), 7.38-7.44 (m, 2H), 7.31 (d, J = 8.07 Hz, 2H), 6.46 (s, 1H), 5.01 (br. s., 1H), 3.65-3.73 (m, 2H), 2.82 (t, J = 7.27 Hz, 2H), 2.43 (d, J = 0.86 Hz, 3H), 2.08 (quin, J = 7.03 Hz, 2H); LCMS: ESI [M + H]⁺ = 428.1 |
| 436 | 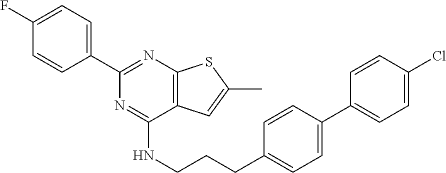 [SMILES: Cc1cc2c(NCCCc3ccc(-c4ccc(Cl)cc4)cc3)nc(-c3ccc(F)cc3)nc2s1] | KH 5015 | A | Method A 1 d (90° C.) | 47% | N-(3-(4′-chloro- [1,1′-biphenyl]-4- yl)propyl)-2-(4- fluorophenyl)-6- methylthieno[2,3- d]pyrimidin-4- amine | ¹H NMR (400 MHz, CHLOROFORM-d) δ 8.45 (dd, J = 5.93, 8.25 Hz, 2H), 7.48- 7.57 (m, 4H), 7.40-7.45 (m, 2H), 7.33 (d, J = 8.07 Hz, 2H), 7.12 (t, J = 8.62 Hz, 2H), 6.60 (s, 1H), 4.93 (br. s., 1H), 3.82 (q, J = 6.52 Hz, 2H), 2.86 (t, J = 7.34 Hz, 2H), 2.51 (s, 3H), 2.15 (quin, J = 7.09 Hz, 2H); LCMS: ESI [M + H]⁺ = 488.2 |
| 437 | 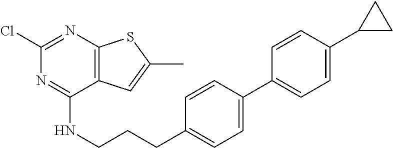 [SMILES: Cc1cc2c(NCCCc3ccc(-c4ccc(C5CC5)cc4)cc3)nc(Cl)nc2s1] | 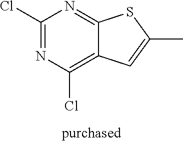 | A | Method A 1 d (40° C.) | 23% | 2-chloro-N-(3-(4′- cyclopropyl-[1,1′- biphenyl]-4- yl)propyl)-6- methylthieno[2,3- d]pyrimidin-4- amine | 1H NMR (400 MHz, CHLOROFORM-d) δ 7.51 (dd, J = 8.13, 19.13 Hz, 4H), 7.30 (d, J = 8.07 Hz, 2H), 7.15 (d, J = 8.19 Hz, 2H), 6.39 (s, 1H), 4.94 (br. s., 1H), 3.70 (q, J = 6.52 Hz, 2H), 2.82 (t, J = 7.15 Hz, 2H), 2.39 (s, 3H), 2.04-2.16 (m, 2H), 1.88-2.00 (m, 1H), |
| | | | | | | | 0.95-1.05 (m, 2H), 0.68-0.79 |
| | | | | | | | (m, 2H); LCMS: ESI |
| | | | | | | | [M + H]⁺ = 434.2 |
| 438 | 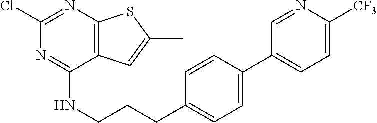 [SMILES: Cc1cc2c(NCCCc3ccc(-c4ccc(C(F)(F)F)nc4)cc3)nc(Cl)nc2s1] | 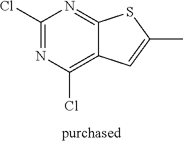 | A | Method A 1 d (40° C.) | 32% | 2-chloro-6-methyl- N-(3-(4-(6- (trifluoromethyl) pyridin-3- yl)phenyl)propyl) thieno[2,3-d] pyrimidin-4-amine | ¹H NMR (400 MHz, CHLOROFORM-d) δ 8.93 (s, 1H), 8.02 (d, J = 8.19 Hz, 1H), 7.76 (d, J = 8.07 Hz, 1H), 7.55 (d, J = 8.07 Hz, 2H), 7.38 (d, J = 8.07 Hz, 2H), 6.61 (s, 1H), 5.08-5.26 (m, 1H), 3.70 (q, J = 6.64 Hz, 2H), 2.84 (t, J = 7.40 Hz, 2H), 2.49 (s, 3H), 2.09 |
| | | | | | | | (quin, J = 7.18 Hz, 2H); |
| | | | | | | | LCMS: ESI [M + H]⁺ = 463.1 |
| 439 | 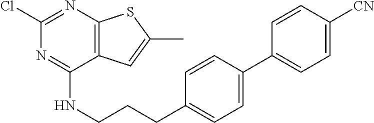 [SMILES: Cc1cc2c(NCCCc3ccc(-c4ccc(C#N)cc4)cc3)nc(Cl)nc2s1] | 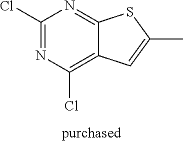 | A | Method A 1 d (40° C.) | 21% | 4′-(3-((2-chloro-6- methylthieno[2,3- d]pyrimidin-4- yl)amino)propyl)- [1,1′-biphenyl]-4- carbonitrile | ¹H NMR (400 MHz, CHLOROFORM-d) δ 7.64-7.77 (m, 4H), 7.54 (d, J = 8.07 Hz, 2H), 7.35 (d, J = 8.07 Hz, 2H), 6.55 (s, 1H), 5.09 (br. s., 1H), 3.70 (q, J = 6.64 Hz, 2H), 2.83 (t, J = 7.34 Hz, 2H), 2.46 (s, 3H), 2.09 (quin, J = 7.12 Hz, 2H); LCMS: ESI [M + H]⁺ = 419.1 |
| 440 | 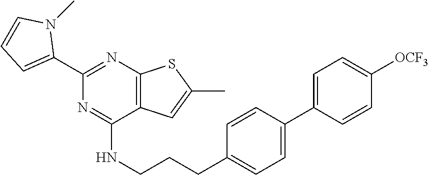 [SMILES: Cc1cc2c(NCCCc3ccc(-c4ccc(OC(F)(F)F)cc4)cc3)nc(-c3cccn3C)nc2s1] | KH 8088 | A | Method A 3 d (90° C.) | 7% | 6-methyl-2-(1- methyl-1H-pyrrol- 2-yl)-N-(3-(4′- (trifluoromethoxy)- [1,1′-biphenyl]-4- yl)propyl)thieno [2,3-d]pyrimidin-4- amine | ¹H NMR (400 MHz, METHANOL-d4) δ 6.12 (d, J = 8.80 Hz, 2H), 5.97 (d, J = 8.31 Hz, 2H), 5.77 (dd, J = 8.50, 11.07 Hz, 4H), 5.45 (d, J = 1.22 Hz, 1H), 5.37 (dd, J = 1.90, 3.85 Hz, 1H), 5.18 (t, J = 2.14 Hz, 1H), 4.53 (dd, J = 2.57, 3.79 Hz, 1H), 2.42 (s, 3H), 2.11 (t, J = 7.15 Hz, 2H), 1.25 (t, J = 7.40 Hz, 2H), 0.97 (d, J = 1.22 Hz, |
| | | | | | | | 3H), 0.54 (quin, J = 7.18 Hz, |
| | | | | | | | 2H); LCMS: ESI [M + H]⁺ = 523.1 |
| 441 | 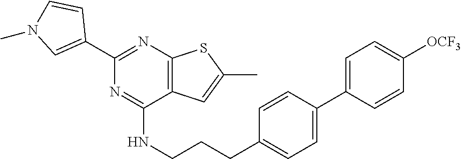 | KH 8098 | A | Method A 3 d (90° C.) | 8% | 6-methyl-2-(1- methyl-1H-pyrrol- 3-yl)-N-(3-(4′- (trifluoromethoxy)- [1,1′-biphenyl]-4- yl)propyl)thieno [2,3-d]pyrimidin- 4-amine | ¹H NMR (400 MHz, METHANOL-d4) δ 6.92 (s, 1H), 6.12 (d, J = 8.68 Hz, 2H), 5.98 (d, J = 8.07 Hz, 2H), 5.71- 5.85 (m, 5H), 5.42 (s, 1H), 5.11- 5.20 (m, 1H), 5.07 (t, J = 2.45 Hz, 1H), 2.15 (t, J = 6.97 Hz, 2H), 2.09 (s, 3H), 1.26 (t, J = 7.34 Hz, 2H), 0.96 (s, 3H), 0.55 (quin, J = 7.21 Hz, 2H); LCMS: |
| | | | | | | | ESI [M + H]⁺ = 523.2 |
| 442 | 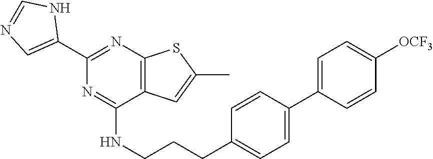 [SMILES: Cc1cc2c(NCCCc3ccc(-c4ccc(OC(F)(F)F)cc4)cc3)nc(-c3cnc[nH]3)nc2s1] | KH 8099 | A | Method A 3 d (90° C.) | 9% | 2-(1H-imidazol-5- yl)-6-methyl-N- (3-(4′- (trifluoromethoxy)- [1,1′-biphenyl]-4- yl)propyl)thieno [2,3-d]pyrimidin- 4-amine | ¹H NMR (400 MHz, METHANOL-d4) δ 6.75 (s, 1H), 6.29-6.36 (m, 1H), 6.18 (d, J = 0.98 Hz, 1H), 6.10 (d, J = 8.80 Hz, 2H), 5.95 (d, J = 8.19 Hz, 2H), 5.76 (dd, J = 2.38, 8.50 Hz, 4H), 5.48 (d, J = 1.22 Hz, 1H), 2.22 (t, J = 6.91 Hz, 2H), 1.27 (t, J = 7.46 Hz, 2H), 0.97 (s, 3H), 0.55 (quin, J = |
| | | | | | | | 7.21 Hz, 2H); LCMS: ESI |
| | | | | | | | [M + H]⁺ = 510.1 |
| 443 | 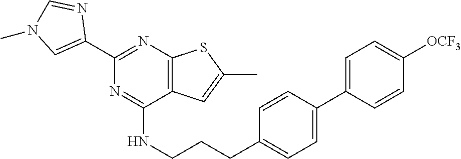 | KH 9003 | A | Method A 3 d (90° C.) | 17% | 6-methyl-2-(1- methyl-1H- imidazol-4-yl)-N- (3-(4′- (trifluoromethoxy)- [1,1′-biphenyl]-4- yl)propyl)thieno [2,3-d]pyrimidin- 4-amine | ¹H NMR (400 MHz, METHANOL-d4) δ 6.84 (s, 1H), 6.21 (s, 1H), 6.07-6.15 (m, 3H), 5.96 (d, J = 8.19 Hz, 2H), 5.77 (dd, J = 3.48, 8.01 Hz, 4H), 5.47 (d, J = 1.22 Hz, 1H), 2.14-2.27 (m, 5H), 1.26 (t, J = 7.46 Hz, 2H), 0.97 (d, J = 1.10 Hz, 3H), 0.54 (quin, J = 7.21 Hz, 2H); LCMS: ESI |
| | | | | | | | [M + H]⁺ = 524.2 |
| 444 | 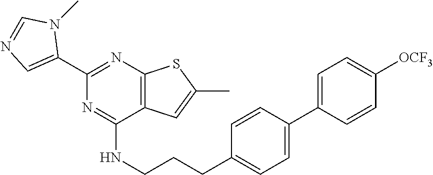 [SMILES: Cc1cc2c(NCCCc3ccc(-c4ccc(OC(F)(F)F)cc4)cc3)nc(-c3cncn3C)nc2s1] | KH 9004 | A | Method A 3 d (90° C.) | 16% | 6-methyl-2-(1- methyl-1H- imidazol-5-yl)-N- (3-(4′- (trifluoromethoxy)- [1,1′-biphenyl]-4- yl)propyl)thieno [2,3-d]pyrimidin- 4-amine | ¹H NMR (399 MHz, METHANOL-d4) δ 7.62-7.70 (m, 4H), 7.50 (d, J = 8.17 Hz, 2H), 7.29 (d, J = 7.79 Hz, 2H), 7.32 (d, J = 8.18 Hz, 2H), 7.03 (s, 1H), 3.98 (s, 3H), 3.64 (t, J = 7.01 Hz, 2H), 2.79 (t, J = 7.40 Hz, 2H), 2.53 (s, 3H), 2.08 (quin, J = 7.10 Hz, 2H); LCMS: ESI [M + H]⁺ = 524.2 |
| 445 | 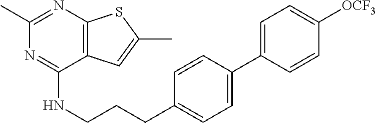 [SMILES: Cc1nc(NCCCc2ccc(-c3ccc(OC(F)(F)F)cc3)cc2)c2cc(C)sc2n1] | 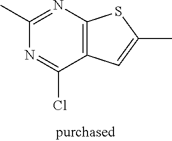 | A | Method A 1 d (90° C.) | 15% | 2,6-dimethyl-N- (3-(4′- (trifluoromethoxy)- [1,1′-biphenyl]-4- yl)propyl)thieno [2,3-d]pyrimidin- 4-amine | ¹H NMR (399 MHz, METHANOL-d4) δ 7.67 (d, J = 8.95 Hz, 2H), 7.52 (d, J = 8.17 Hz, 2H), 7.32 (dd, J = 2.92, 7.98 Hz, 4H), 6.99 (s, 1H), 3.61 (t, J = 7.01 Hz, 2H), 2.77 (t, J = 7.40 Hz, 2H), 2.50 (s, 3H), 2.48 (s, 3H), 2.05 (quin, J = 7.30 Hz, |
| | | | | | | | 2H); LCMS: ESI [M + H]⁺ = 458.1 |
| 446 | 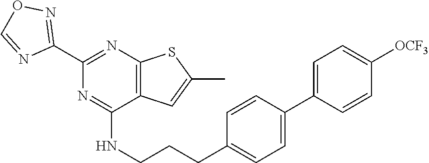 [SMILES: Cc1cc2c(NCCCc3ccc(-c4ccc(OC(F)(F)F)cc4)cc3)nc(-c3ncon3)nc2s1] | KH 8054 | A | Method A 1 d (90° C.) | 11% | 6-methyl-2-(1,2,4- oxadiazol-3-yl)-N- (3-(4′- (trifluoromethoxy)- [1,1′-biphenyl]-4- yl)propyl)thieno [2,3-d]pyrimidin- 4-amine | ¹H NMR (399 MHz, METHANOL-d4) δ 9.43 (s, 1H), 7.65 (d, J = 8.56 Hz, 2H), 7.49 (d, J = 8.17 Hz, 2H), 7.32 (d, J = 8.17 Hz, 4H), 7.12 (s, 1H), 3.76 (t, J = 6.81 Hz, 2H), 2.81 (t, J = 7.59 Hz, 2H), 2.56 (s, 3H), 2.10 (quin, J = 7.20 Hz, 2H); LCMS: ESI [M + H]⁺ = 512.1 |
| 447 | 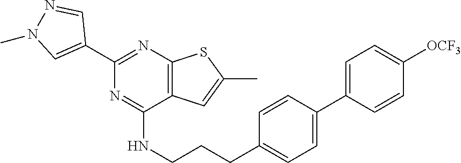 [SMILES: Cc1cc2c(NCCCc3ccc(-c4ccc(OC(F)(F)F)cc4)cc3)nc(-c3cnn(C)c3)nc2s1] | KH 7079 | A | Method A 1 d (90° C.) | 26% | 6-methyl-2-(1- methyl-1H- pyrazol-4-yl)-N- (3-(4′- (trifluoromethoxy)- [1,1′-biphenyl]-4- yl)propyl)thieno [2,3-d]pyrimidin- 4-amine | ¹H NMR (399 MHz, METHANOL-d4) δ 8.02 (s, 1H), 8.05 (s, 1H), 7.65 (d, J = 8.95 Hz, 2H), 7.51 (d, J = 8.17 Hz, 2H), 7.26-7.34 (m, 4H), 7.00 (s, 1H), 3.87 (s, 3H), 3.68 (t, J = 7.01 Hz, 2H), 2.79 (t, J = 7.40 Hz, 2H), 2.51 (s, 3H), 2.08 (quin, J = 7.20 Hz, 2H); LCMS: ESI [M + H]⁺ = 524.2 |
| 448 | 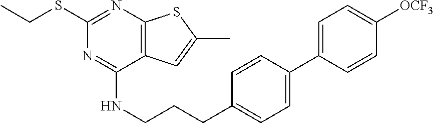 [SMILES: CCSc1nc(NCCCc2ccc(-c3ccc(OC(F)(F)F)cc3)cc2)c2cc(C)sc2n1] | KH 5047 | A | Method A 1 d (90° C.) | 25% | 2-(ethylthio)-6- methyl-N-(3-(4′- (trifluoromethoxy)- [1,1′-biphenyl]-4- yl)propyl)thieno [2,3-d]pyrimidin- 4-amine | ¹H NMR (399 MHz, METHANOL-d4) δ 7.67 (d, J = 8.56 Hz, 2H), 7.52 (d, J = 8.18 Hz, 2H), 7.31 (t, J = 9.34 Hz, 4H), 6.93 (s, 1H), 3.61 (t, J = 7.01 Hz, 2H), 3.05 (q, J = 7.14 Hz, 2H), 2.77 (t, J = 7.59 Hz, 2H), 2.47 (s, 3H), 2.05 (quin, |
| | | | | | | | J = 7.20 Hz, 2H), 1.32 (t, J = |
| | | | | | | | 7.20 Hz, 3H); LCMS: ESI |
| | | | | | | | [M + H]⁺ = 504.1 |
| 449 | 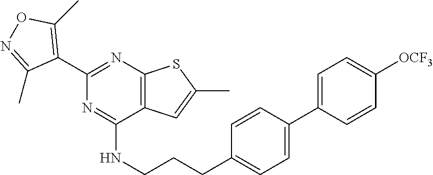 [SMILES: Cc1cc2c(NCCCc3ccc(-c4ccc(OC(F)(F)F)cc4)cc3)nc(-c3c(C)noc3C)nc2s1] | KH 9049 | A | Method A 1 h (140° C.) microwave | 30% | 2-(3,5- dimethylisoxazol- 4-yl)-6-methyl-N- (3-(4′- (trifluoromethoxy)- [1,1′-biphenyl]-4- yl)propyl)thieno [2,3-d]pyrimidin- 4-amine | ¹H NMR (399 MHz, DMSO-d6) δ 7.90 (t, J = 5.26 Hz, 1H), 7.76 (d, J = 8.56 Hz, 2H), 7.55-7.63 (m, J = 8.17 Hz, 2H), 7.40-7.48 (m, J = 8.95 Hz, 2H), 7.33 (d, J = 8.18 Hz, 2H), 7.27 (s, 1H), 3.55 (q, J = 6.88 Hz, 2H), 2.74 (t, J = 7.59 Hz, 2H), 2.70 (s, 3H), 2.52 (s, 3H), 2.46 (s, 3H), 1.97 (td, J = 7.49, 14.60 Hz, 2H); LCMS: ESI [M + H]⁺ = 539.1 |
| 450 | 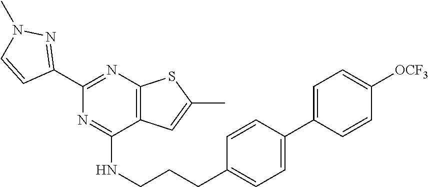 [SMILES: Cc1cc2c(NCCCc3ccc(-c4ccc(OC(F)(F)F)cc4)cc3)nc(-c3ccn(C)n3)nc2s1] | KH 9051 | A | Method A 1 h (140° C.) microwave | 25% | 6-methyl-2-(1- methyl-1H- pyrazol-3-yl)-N- (3-(4′- (trifluoromethoxy)- [1,1′-biphenyl]-4- yl)propyl)thieno [2,3-d]pyrimidin- 4-amine | ¹H NMR (399 MHz, METHANOL-d4) δ 7.64 (d, J = 8.56 Hz, 2H), 7.56 (s, 1H), 7.49 (d, J = 8.18 Hz, 2H), 7.31 (t, J = 7.79 Hz, 4H), 7.03 (s, 1H), 6.84 (s, 1H), 3.96 (s, 3H), 3.74 (t, J = 6.81 Hz, 2H), 2.79 (t, J = 7.40 Hz, 2H), 2.52 (s, 3H), 2.07 (quin, J = 7.30 Hz, 2H); LCMS: ESI [M + H]⁺ = 524.2 |
| 451 | 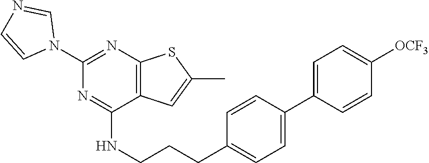 [SMILES: Cc1cc2c(NCCCc3ccc(-c4ccc(OC(F)(F)F)cc4)cc3)nc(-n3ccnc3)nc2s1] | KH 7080 | A | Method A 1 d (130° C.) pressure vessel | 43% | 2-(1H-imidazol-1- yl)-6-methyl-N-(3- (4′- (trifluoromethoxy)- [1,1′-biphenyl]-4- yl)propyl)thieno [2,3-d]pyrimidin- 4-amine | ¹H NMR (399 MHz, DMSO-d6) δ 8.46 (s, 1H), 8.29 (t, J = 5.06 Hz, 1H), 7.74-7.83 (m, 3H), 7.56-7.65 (m, J = 7.79 Hz, 2H), 7.40-7.49 (m, J = 7.79 Hz, 2H), 7.36 (d, J = 7.79 Hz, 2H), 7.29 (s, 1H), 7.01 (s, 1H), 3.59 (q, J = 6.62 Hz, 2H), 2.76 (t, J = 7.20 Hz, 2H), 1.93-2.04 (m, 2H); LCMS: ESI [M + H]⁺ = 510.1 |
| 452 | 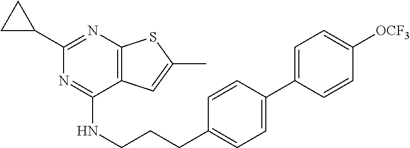 [SMILES: Cc1cc2c(NCCCc3ccc(-c4ccc(OC(F)(F)F)cc4)cc3)nc(C3CC3)nc2s1] | KH 4082 | A | Method A 3 d (85° C.) | 17% | 2-cyclopropyl-6- methyl-N-(3-(4′- (trifluoromethoxy)- [1,1′-bipheny]]-4- yl)propyl)thieno [2,3-d]pyrimidin- 4-amine | ¹H NMR (399 MHz, METHANOL-d4) δ 7.67 (d, J = 8.56 Hz, 2H), 7.52 (d, J = 8.17 Hz, 2H), 7.29 (d, J = 8.17 Hz, 2H), 7.32 (d, J = 8.56 Hz, 2H), 6.93 (s, 1H), 3.55 (t, J = 7.20 Hz, 2H), 2.74 (t, J = 7.40 Hz, 2H), 2.48 (s, 3H), 1.91-2.06 (m, 3H), 1.02 (br. s., 2H), 0.86-0.94 |
| | | | | | | | (m, 2H); LCMS: ESI |
| | | | | | | | [M + H]⁺ = 484.2 |
| 453 | 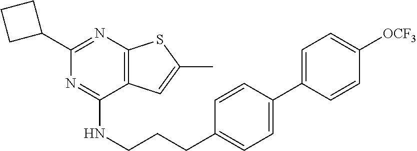 [SMILES: Cc1cc2c(NCCCc3ccc(-c4ccc(OC(F)(F)F)cc4)cc3)nc(C3CCC3)nc2s1] | KH 6091 | A | Method A 3 d (85° C.) | 24% | 2-cyclobutyl-6- methyl-N-(3-(4′- (trifluoromethoxy)- [1,1′-biphenyl]-4- yl)propyl)thieno [2,3-d]pyrimidin- 4-amine | ¹H NMR (399 MHz, METHANOL-d4) δ 7.65 (d, J = 8.56 Hz, 2H), 7.50 (d, J = 7.79 Hz, 2H), 7.30 (t, J = 7.40 Hz, 4H), 6.97 (s, 1H), 3.67 (t, J = 7.01 Hz, 2H), 3.56 (quin, J = 8.56 Hz, 1H), 2.78 (t, J = 7.40 Hz, 2H), 2.49 (s, 3H), 2.36-2.46 (m, 2H), 2.27 (q, J = 8.56 Hz, |
| | | | | | | | 2H), 1.83-2.12 (m, 4H); LCMS: |
| | | | | | | | ESI [M + H]⁺ = 498.2 |
| 454 | 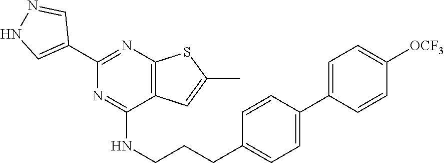 [SMILES: Cc1cc2c(NCCCc3ccc(-c4ccc(OC(F)(F)F)cc4)cc3)nc(-c3cn[nH]c3)nc2s1] | KH 7078 | A | Method A 1 d (100° C.) | 5% | 6-methyl-2-(1H- pyrazol-4-yl)-N- (3-(4′- (trifluoromethoxy)- [1,1′-biphenyl]-4- yl)propyl)thieno [2,3-d]pyrimidin- 4-amine | ¹H NMR (399 MHz, METHANOL-d4) δ 8.18 (br. s., 2H), 7.66 (d, J = 8.17 Hz, 2H), 7.51 (d, J = 7.79 Hz, 2H), 7.32 (d, J = 7.79 Hz, 4H), 7.01 (s, 1H), 3.70 (t, J = 6.81 Hz, 2H), 2.81 (t, J = 7.40 Hz, 2H), 2.52 (s, 3H), 2.11 (td, J = 7.06, 14.31 Hz, 2H); LCMS: ESI [M + H]⁺ = 510.2 |
| 455 | 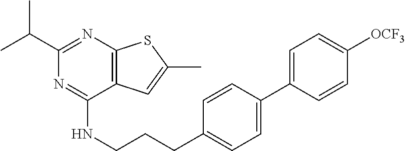 [SMILES: Cc1cc2c(NCCCc3ccc(-c4ccc(OC(F)(F)F)cc4)cc3)nc(C(C)C)nc2s1] | KH 9078 | A | Method A 1 d (100° C.) | 6% | 2-isopropyl-6- methyl-N-(3-(4′- (trifluoromethoxy)- [1,1′-biphenyl]-4- yl)propyl)thieno [2,3-d]pyrimidin- 4-amine | ¹H NMR (399 MHz, METHANOL-d4) δ 7.66 (d, J = 8.56 Hz, 2H), 7.51 (d, J = 8.18 Hz, 2H), 7.31 (t, J = 8.37 Hz, 4H), 6.97 (s, 1H), 3.64 (t, J = 7.01 Hz, 2H), 2.95 (td, J = 6.81, 13.62 Hz, 1H), 2.77 (t, J = 7.20 Hz, 2H), 2.50 (s, 3H), 2.06 (quin, J = 7.30 Hz, 2H), 1.28 (d, |
| | | | | | | | J = 7.01 Hz, 6H); LCMS: ESI |
| | | | | | | | [M + H]⁺ = 486.2 |
| 456 | 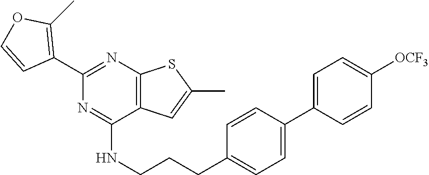 [SMILES: Cc1cc2c(NCCCc3ccc(-c4ccc(OC(F)(F)F)cc4)cc3)nc(-c3ccoc3C)nc2s1] | KH 9091 | A | Method A 1 h (150° C.) microwave | 14% | 6-methyl-2-(2- methylfuran-3-yl)- N-(3-(4′- (trifluoromethoxy)- [1,1′-biphenyl]-4- yl)propyl)thieno [2,3-d]pyrimidin- 4-amine | ¹H NMR (399 MHz, METHANOL-d4) δ 7.65 (d, J = 8.56 Hz, 2H), 7.49 (d, J = 7.79 Hz, 2H), 7.25-7.36 (m, 5H), 7.00 (s, 1H), 6.92 (s, 1H), 3.66 (t, J = 7.20 Hz, 2H), 2.73-2.83 (m, J = 7.40, 7.40 Hz, 2H), 2.64 (s, 3H), 2.52 (s, 3H), 2.07 (quin, J = 7.20 Hz, 2H); LCMS: ESI [M + H]⁺ = 524.2 |
| 457 | 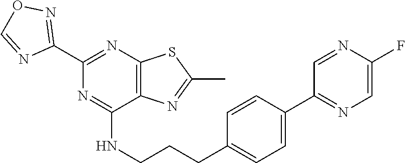 [SMILES: Cc1nc2c(NCCCc3ccc(-c4cnc(F)cn4)cc3)nc(-c3ncon3)nc2s1] | KH 9100 | A | Method A 2 h (50° C.) | 23% | N-(3-(4-(5- fluoropyrazin-2- yl)phenyl)propyl)- 2-methyl-5-(1,2,4- oxadiazol-3- yl)thiazolo[5,4-d] pyrimidin-7-amine | ¹H NMR (399 MHz, DMSO-d6) δ 8.91 (s, 1H), 8.05 (s, 1H), 7.90 (d, J = 8.56 Hz, 1H), 7.69 (t, J = 5.45 Hz, 1H), 7.11-7.17 (m, J = 7.79 Hz, 2H), 6.54-6.61 (m, J = 8.17 Hz, 2H), 2.76 (q, J = 6.23 Hz, 2H), 1.99 (s, 3H), 1.89 (t, J = 7.40 Hz, 2H), 1.18 (quin, J = 7.10 Hz, 2H); LCMS: ESI [M + H]⁺ = 449.1 |
| 458 | 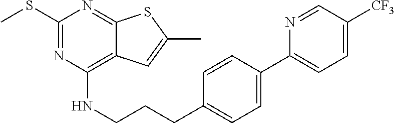 [SMILES: CSc1nc(NCCCc2ccc(-c3ccc(C(F)(F)F)cn3)cc2)c2cc(C)sc2n1] | KH 5002 | A | Method A 1 h (140° C.) microwave | 8% | 6-methyl-2- (methylthio)-N-(3- (4-(5-(trifluoro- methyl)pyridin-2- yl)phenyl)propyl) thieno[2,3- d]pyrimidin-4- amine | 1H NMR (399 MHz, METHANOL-d4) δ 8.90 (s, 1H), 8.14 (d, J = 8.95 Hz, 1H), 7.96-8.05 (m, 3H), 7.37 (d, J = 8.17 Hz, 2H), 6.93 (s, 1H), 3.63 (t, J = 7.01 Hz, 2H), 2.81 (t, J = 7.40 Hz, 2H), 2.46 (d, J = 8.56 Hz, 6H), 2.07 (quin, J = 7.20 |
| | | | | | | | Hz, 2H); LCMS: ESI |
| | | | | | | | [M + H]⁺ = 475.1 |
| 459 | 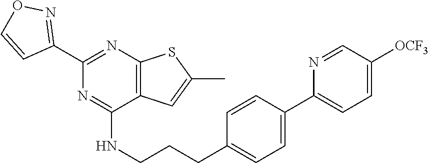 [SMILES: Cc1cc2c(NCCCc3ccc(-c4ccc(OC(F)(F)F)cn4)cc3)nc(-c3ccon3)nc2s1] | KH 7018 | A | Method A 1 d (120° C.) | 10% | 2-(isoxazol-3-yl)- 6-methyl-N-(3-(4- (5- (trifluoromethoxy) pyridin-2- yl)phenyl)propyl) thieno[2,3- d]pyrimidin-4- amine | ¹H NMR (399 MHz, METHANOL-d4) δ 8.73 (s, 1H), 8.58 (br. s., 1H), 7.86-7.96 (m, 3H), 7.82 (d, J = 8.95 Hz, 1H), 7.36 (d, J = 7.79 Hz, 2H), 7.10 (s, 1H), 6.95 (s, 1H), 3.75 (t, J = 7.01 Hz, 2H), 2.83 (t, J = 7.59 Hz, 2H), 2.55 (s, 3H), 2.11 (quin, J = 7.20 Hz, 2H); LCMS: ESI [M + H]⁺ = 512.1 |
| 460 | 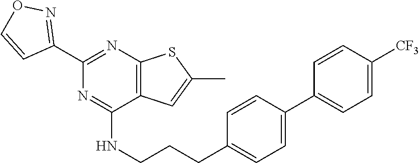 [SMILES: Cc1cc2c(NCCCc3ccc(-c4ccc(C(F)(F)F)cc4)cc3)nc(-c3ccon3)nc2s1] | KH 7018 | A | Method A 3 d (90° C.) | 19% | 2-(isoxazol-3-yl)- 6-methyl-N-(3-(4′- (trifluoromethyl)- [1,1′-biphenyl]-4- yl)propyl)thieno [2,3-d]pyrimidin- 4-amine | ¹H NMR (400 MHz, CHLOROFORM-d) δ 8.45 (s, 1H), 7.69 (s, 4H), 7.50-7.59 (m, J = 7.79 Hz, 2H), 7.31-7.39 (m, J = 7.79 Hz, 2H), 6.96 (s, 1H), 6.63 (s, 1H), 5.04 (br. s., 1H), 3.76-3.86 (m, 2H), 2.85 (t, J = 7.20 Hz, 2H), 2.50 (s, 3H), 2.12 (t, J = 7.01 Hz, 2H); LCMS: ESI [M + H]⁺ = 495.1 |
| 461 | 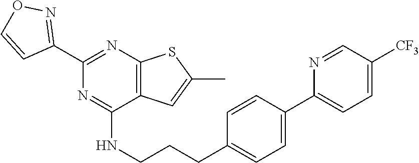 [SMILES: Cc1cc2c(NCCCc3ccc(-c4ccc(C(F)(F)F)cn4)cc3)nc(-c3ccon3)nc2s1] | KH 7018 | A | Method A 1 h (140° C.) microwave | 17% | 2-(isoxazol-3-yl)- 6-methyl-N-(3-(4- (5- (trifluoromethyl) pyridin-2- yl)phenyl)propyl) thieno[2,3-d] pyrimidin-4-amine | ¹H NMR (400 MHz, CHLOROFORM-d) δ 8.95 (s, 1H), 8.46 (s, 1H), 7.99 (d, J = 7.79 Hz, 3H), 7.83 (d, J = 8.17 Hz, 1H), 7.37 (d, J = 7.79 Hz, 2H), 6.96 (s, 1H), 6.65 (s, 1H), 5.08 (br. s., 1H), 3.80 (q, J = 6.23 Hz, 2H), 2.86 (t, J = 7.40 Hz, 2H), 2.50 (s, 3H), 2.12 (quin, J = 7.10 Hz, 2H); |
| | | | | | | | LCMS: ESI [M + H]⁺ = 496.1 |
| 462 | 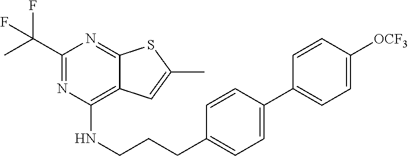 [SMILES: Cc1cc2c(NCCCc3ccc(-c4ccc(OC(F)(F)F)cc4)cc3)nc(C(C)(F)F)nc2s1] | KH 4100 | A | Method A 1 h (150° C.) microwave | 17% | 2-(1,1- difluoroethyl)-6- methyl-N-(3-(4′- (trifluoromethoxy)- [1,1′-biphenyl]-4- yl)propyl)thieno [2,3-d]pyrimidin- 4-amine | ¹H NMR (400 MHz, CHLOROFORM-d) δ 7.56 (d, J = 7.40 Hz, 2H), 7.48 (d, J = 7.40 Hz, 2H), 7.27 (t, J = 8.56 Hz, 4H), 6.55 (s, 1H), 4.99 (br. s., 1H), 3.70 (q, J = 6.23 Hz, 2H), 2.79 (t, J = 7.20 Hz, 2H), 2.45 (s, 3H), 1.94-2.12 (m, 5H); LCMS: ESI [M + H]⁺ = 508.1 |
| 463 | 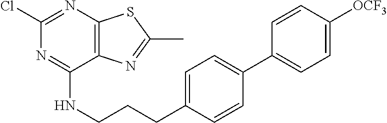 [SMILES: Cc1nc2c(NCCCc3ccc(-c4ccc(OC(F)(F)F)cc4)cc3)nc(Cl)nc2s1] | 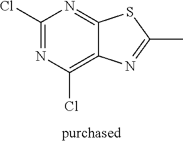 | A | Method A 1 d (50° C.) | 99% | 5-chloro-2-methyl- N-(3-(4′- (trifluoromethoxy)- [1,1′-biphenyl]-4- yl)propyl)thiazolo [5,4-d]pyrimidin- 7-amine | LCMS: ESI [M + H]⁺ = 479.1 |
| 464 | 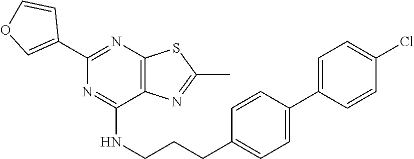 [SMILES: Cc1nc2c(NCCCc3ccc(-c4ccc(Cl)cc4)cc3)nc(-c3ccoc3)nc2s1] | LM 13028 | A | Method A 2 d (60° C.) | 40% | N-(3-(4′-chloro- [1,1′-biphenyl]-4- yl)propyl)-5- (furan-3-yl)-2- methylthiazolo [5,4-d]pyrimidin- 7-amine | ¹H NMR (399 MHz, CHLOROFORM-d) δ 8.14 (s, 1H), 7.50 (t, J = 8.56 Hz, 4H), 7.37-7.46 (m, 3H), 7.30 (d, J = 8.17 Hz, 2H), 7.01 (d, J = 1.17 Hz, 1H), 5.97 (br. s., 1H), 3.75 (q, J = 6.62 Hz, 2H), 2.82 (t, J = 7.40 Hz, 2H), 2.76 (s, 3H), 2.10 (quin, J = 7.20 Hz, 2H); LCMS: ESI [M + H]⁺ = 461.1 |
| 465 | 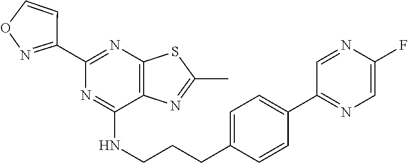 [SMILES: Cc1nc2c(NCCCc3ccc(-c4cnc(F)cn4)cc3)nc(-c3ccon3)nc2s1] | LM 15015 | A | Method A 1 h (50° C.) | 42% | N-(3-[4-(5- fluoropyrazin-2- yl)phenyl]propyl)- 2-methyl-5-(1,2- oxazol-3-yl)- [1,3]thiazolo[5,4- d]pyrimidin-7- amine | LCMS: ESI [M + H]⁺ = 448.1 |
| 466 | 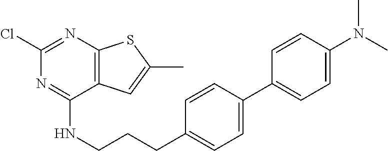 [SMILES: Cc1cc2c(NCCCc3ccc(-c4ccc(N(C)C)cc4)cc3)nc(Cl)nc2s1] | 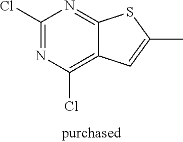 | A | Method B 2 h (45° C.) | 36% | 2-chloro-N-(3-(4′- (dimethylamino)- [1,1′-biphenyl]-4- yl)propyl)-6- methylthieno[2,3- d]pyrimidin-4- amine | LCMS: ESI [M + H]⁺ = 437.2 |
| 467 | 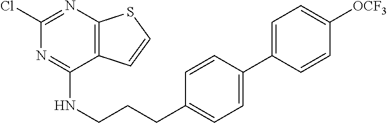 [SMILES: FC(F)(F)Oc1ccc(-c2ccc(CCCNc3nc(Cl)nc4sccc34)cc2)cc1] | 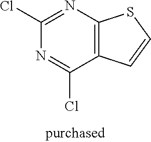 [SMILES: Clc1nc(Cl)c2ccsc2n1] | A | Method B 1 d (45° C.) | 51% | 2-chloro-N-(3-(4′- (trifluoromethoxy)- [1,1′-biphenyl]-4- yl)propyl)thieno [2,3-d]pyrimidin- 4-amine | ¹H NMR (400 MHz, METHANOL-d₄) δ 6.11 (d, J = 8.68 Hz, 2H), 5.97 (d, J = 8.07 Hz, 2H), 5.82-5.88 (m, 2H), 5.78 (dd, J = 2.57, 8.44 Hz, 4H), 2.08 (t, J = 7.09 Hz, 2H), 1.24 (t, J = 7.52 Hz, 2H), 0.52 (quin, J = 7.31 Hz, 2H); |
| | | | | | | | LCMS: ESI [M + H]⁺ = 464.1 |
| 468 | 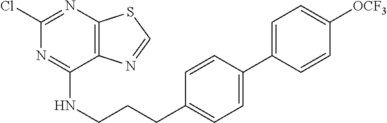 [SMILES: FC(F)(F)Oc1ccc(-c2ccc(CCCNc3nc(Cl)nc4scnc34)cc2)cc1] | 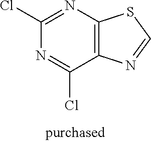 [SMILES: Clc1nc(Cl)c2ncsc2n1] | A | Method B 5 d (45° C.) | 90% | 5-chloro-N-(3-(4′- (trifluoromethoxy)- [1,1′-biphenyl]-4- yl)propyl)thiazolo [5,4-d]pyrimidin- 7-amine | ¹H NMR (400 MHz, METHANOL-d4) δ 7.42 (s, 1H), 6.11 (d, J = 8.68 Hz, 2H), 5.96 (d, J = 8.19 Hz, 2H), 5.78 (d, J = 8.07 Hz, 4H), 2.10 (t, J = 6.97 Hz, 2H), 1.24 (t, J = 7.52 Hz, 2H), 0.53 (quin, J = 7.24 Hz, 2H); LCMS: ESI |
| | | | | | | | [M + H]⁺ = 465.1 |
| 469 | 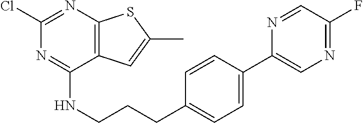 [SMILES: Cc1cc2c(NCCCc3ccc(-c4cnc(F)cn4)cc3)nc(Cl)nc2s1] | 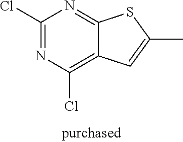 | A | Method B 3 d (45° C.) | 71% | 2-chloro-N-(3-(4- (5-fluoropyrazin- 2- yl)phenyl)propyl)- 6-methylthieno [2,3-d]pyrimidin- 4-amine | ¹H NMR (399 MHz, METHANOL-d4) δ 8.68 (s, 1H), 8.53 (d, J = 8.17 Hz, 1H), 7.88-7.96 (m, J = 7.79 Hz, 2H), 7.34-7.41 (m, J = 8.17 Hz, 2H), 7.01 (s, 1H), 3.60 (t, J = 6.81 Hz, 2H), 2.80 (t, J = 7.40 Hz, 2H), 2.50 (s, 3H), 2.06 (quin, J = 7.20 Hz, |
| | | | | | | | 2H); LCMS: ESI [M + H]⁺ = 414.1 |
| 470 | 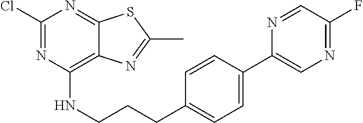 [SMILES: Cc1nc2c(NCCCc3ccc(-c4cnc(F)cn4)cc3)nc(Cl)nc2s1] | 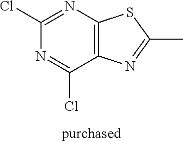 | A | Method B 1 d (45° C.) | 39% | 5-chloro-N-(3-(4- (5-fluoropyrazin-2- yl)phenyl)propyl)- 2-methylthiazolo [5,4-d]pyrimidin- 7-amine | ¹H NMR (399 MHz, ACETONITRILE-d3) δ 8.66 (s, 1H), 8.51 (d, J = 8.56 Hz, 1H), 7.88-7.96 (m, J = 7.79 Hz, 2H), 7.34-7.43 (m, J = 7.79 Hz, 2H), 6.83 (br. s., 1H), 3.57 (q, J = 6.36 Hz, 2H), 2.78 (t, J = 7.40 Hz, 2H), 2.70 (s, 3H), 2.03 (td, J = |
| | | | | | | | 7.49, 14.60 Hz, 2H); LCMS: |
| | | | | | | | ESI [M + H]⁺ = 415.1 |
| 471 | 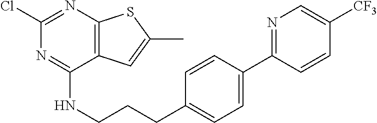 [SMILES: Cc1cc2c(NCCCc3ccc(-c4ccc(C(F)(F)F)cn4)cc3)nc(Cl)nc2s1] | 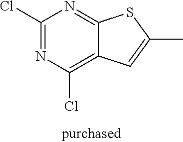 | A | Method B 1 d (45° C.) | 38% | 2-chloro-6-methyl- N-(3-(4-(5- (trifluoromethyl) pyridin-2- yl)phenyl)propyl) thieno[2,3- d]pyrimidin-4- amine | LCMS: ESI [M + H]⁺ = 463.1 |
| 472 | 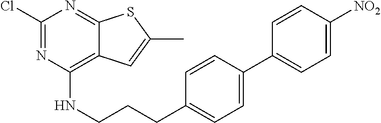 [SMILES: Cc1cc2c(NCCCc3ccc(-c4ccc([N+](=O)[O-])cc4)cc3)nc(Cl)nc2s1] | 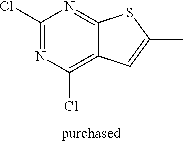 | A | Method B 1 d (45° C.) | 47% | 2-chloro-6-methyl- N-(3-(4′-nitro- [1,1′-biphenyl]-4- yl)propyl)thieno [2,3-d]pyrimidin- 4-amine | LCMS: ESI [M + H]⁺ = 439.1 |
| 473 | 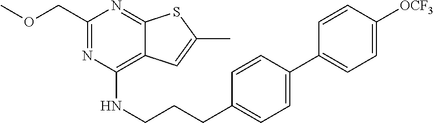 [SMILES: COCc1nc(NCCCc2ccc(-c3ccc(OC(F)(F)F)cc3)cc2)c2cc(C)sc2n1] | KH 5062 | A | Method D 1 d (60° C.) | 62% | 2-(methoxymethyl)- 6-methyl-N-(3-(4′- (trifluoromethoxy)- [1,1′-biphenyl]-4- yl)propyl)thieno [2,3-d]pyrimidin- 4-amine | ¹H NMR (400 MHz, CHLOROFORM-d) δ 7.59 (d, J = 8.56 Hz, 2H), 7.51 (d, J = 8.18 Hz, 2H), 7.30 (t, J = 9.15 Hz, 5H), 6.55 (br. s., 1H), 4.57 (s, 2H), 3.71 (q, J = 6.62 Hz, 2H), 3.55 (s, 3H), 2.82 (t, J = 7.40 Hz, 2H), 2.46 (s, 3H), |
| | | | | | | | 2.08 (quin, J = 7.01 Hz, 2H); |
| | | | | | | | LCMS: ESI [M + H]⁺ = 488.1 |
| 474 | 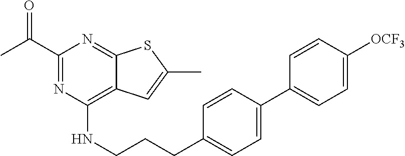 [SMILES: CC(=O)c1nc(NCCCc2ccc(-c3ccc(OC(F)(F)F)cc3)cc2)c2cc(C)sc2n1] | KH 11057 | A | Method D 3 d (60° C.) | 8% | 1-(6-methyl-4-((3- (4′- (trifluoromethoxy)- [1,1′-biphenyl]-4- yl)propyl)amino) thieno[2,3-d] pyrimidin-2- yl)ethan-1-one | ¹H NMR (400 MHz, CHLOROFORM-d) δ 7.56- 7.62 (m, 2H), 7.51 (d, J = 8.17 Hz, 2H), 7.27-7.35 (m, 5H), 6.69 (br. s., 1H), 3.73-3.82 (m, 2H), 2.85 (t, J = 7.20 Hz, 2H), 2.72 (s, 3H), 2.51 (s, 3H), 2.13 (quin, J = 7.01 Hz, 2H); LCMS: ESI [M + H]⁺ = 486.1 |
| 475 | 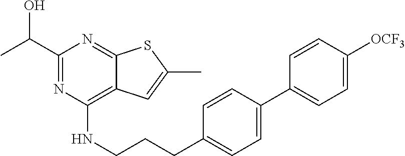 [SMILES: Cc1cc2c(NCCCc3ccc(-c4ccc(OC(F)(F)F)cc4)cc3)nc(C(C)O)nc2s1] | KH 11059 | A | Method D 1 d (60° C.) | 63% | 1-(6-methyl-4-((3- (4′- (trifluoromethoxy)- [1,1′-biphenyl]-4- yl)propyl)amino) thieno[2,3- d]pyrimidin-2- yl)ethan-1-ol | ¹H NMR (400 MHz, CHLOROFORM-d) δ 7.52- 7.58 (m, 2H), 7.47 (d, J = 8.18 Hz, 2H), 7.24 (dd, J = 2.14, 7.98 Hz, 4H), 6.53 (s, 1H), 5.04 (t, J = 5.26 Hz, 1H), 4.76 (q, J = 6.62 Hz, 1H), 3.60-3.71 (m, 2H), 2.77 (t, J = 7.40 Hz, 2H), 2.42 (s, 3H), 2.04 (quin, J = 7.10 Hz, 2H), 1.52 (d, J = |
| | | | | | | | 6.62 Hz, 3H); LCMS: ESI |
| | | | | | | | [M + H]⁺ = 488.1 |
| 476 | 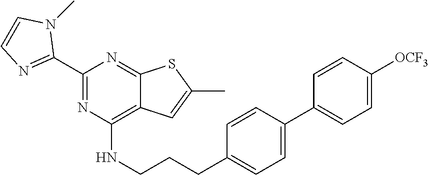 [SMILES: Cc1cc2c(NCCCc3ccc(-c4ccc(OC(F)(F)F)cc4)cc3)nc(-c3nccn3C)nc2s1] | KH 11064 | A | Method D 1 d (60° C.) | 74% | 6-methyl-2-(1H- pyrrol-1-yl)-N-(3- (4′- (trifluoromethoxy)- [1,1′-biphenyl]-4- yl)propyl)thieno [2,3-d]pyrimidin- 4-amine | ¹H NMR (400 MHz, CHLOROFORM-d) δ 8.54 (s, 1H), 7.54-7.60 (m, 2H), 7.46 (d, J = 8.17 Hz, 2H), 7.29 (dd, J = 1.95, 3.89 Hz, 4H), 6.99 (s, 1H), 6.74 (s, 1H), 5.66 (br. s., 1H), 4.10 (s, 3H), 3.68- 3.79 (m, 2H), 2.79 (t, J = 7.40 Hz, 2H), 2.39-2.47 (m, 3H), 2.06 (quin, J = 7.20 Hz, 2H); LCMS: ESI [M + H]⁺ = 524.2 |
| 477 | 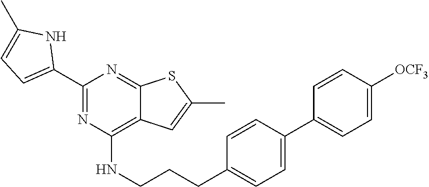 [SMILES: Cc1ccc(-c2nc(NCCCc3ccc(-c4ccc(OC(F)(F)F)cc4)cc3)c3cc(C)sc3n2)[nH]1] | KH 11067 | A | Method D 3 d (60° C.) | 26% | 6-methyl-2-(5- methyl-1H-pyrrol- 2-yl)-N-(3-(4′- (trifluoromethoxy)- [1,1′-biphenyl]-4- yl)propyl)thieno [2,3-d]pyrimidin- 4-amine | ¹H NMR (400 MHz, CHLOROFORM-d) δ 7.56- 7.63 (m, 2H), 7.51 (d, J = 8.18 Hz, 2H), 7.30 (dd, J = 8.56, 12.07 Hz, 4H), 6.92 (br. s., 1H), 6.52 (s, 1H), 5.97 (br. s., 1H), 4.84 (br. s., 1H), 3.76 (q, J = 6.62 Hz, 2H), 2.84 (t, J = 7.20 Hz, 2H), 2.45 (s, 3H), 2.33 (s, 3H), 2.11 (quin, J = 7.01 Hz, 2H); LCMS: ESI |
| | | | | | | | [M + H]⁺ = 523.2 |
| 478 | 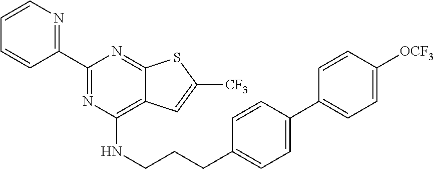 [SMILES: FC(F)(F)Oc1ccc(-c2ccc(CCCNc3nc(-c4ccccn4)nc4sc(C(F)(F)F)cc34)cc2)cc1] | KH 4084 | A | Method D 6 d (60° C.) | 70% | 2-(pyridin-2-yl)- N-(3-(4′- (trifluoromethoxy)- [1,1′-biphenyl]-4- yl)propyl)-6- (trifluoromethyl) thieno[2,3-d] pyrimidin-4-amine | ¹H NMR (400 MHz, CHLOROFORM-d) δ 8.85 (d, J = 3.89 Hz, 1H), 8.48 (d, J = 7.79 Hz, 1H), 7.81 (dt, J = 1.56, 7.79 Hz, 1H), 7.53-7.59 (m, 2H), 7.43-7.53 (m, 3H), 7.36-7.43 (m, 1H), 7.29 (d, J = 7.79 Hz, 4H), 5.65 (br. s., 1H), 3.87 (q, J = 6.62 Hz, 2H), 2.85 (t, J = 7.20 Hz, 2H), 2.15 |
| | | | | | | | (quin, J = 7.10 Hz, 2H); |
| | | | | | | | LCMS: ESI [M + H]⁺ = 575.1 |
| 479 | 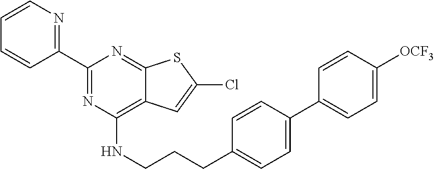 [SMILES: FC(F)(F)Oc1ccc(-c2ccc(CCCNc3nc(-c4ccccn4)nc4sc(Cl)cc34)cc2)cc1] | KH 4037 | A | Method D 6 d (60° C.) | 52% | 6-chloro-2- (pyridin-2-yl)-N- (3-(4′- (trifluoromethoxy)- [1,1′-biphenyl]-4- yl)propyl)thieno [2,3-d]pyrimidin- 4-amine | ¹H NMR (400 MHz, CHLOROFORM-d) δ 8.64- 8.86 (m, 1H), 8.44 (d, J = 8.17 Hz, 1H), 7.79 (dt, J = 1.95, 7.79 Hz, 1H), 7.58 (d, J = 8.56 Hz, 2H), 7.49 (d, J = 8.17 Hz, 2H), 7.19-7.43 (m, 5H), 6.87 (s, 1H), 5.30 (br. s., 1H), 3.82 (q, J = 6.23 Hz, 2H), 2.84 (t, J = 7.40 Hz, 2H), |
| | | | | | | | 2.13 (quin, J = 7.10 Hz, 2H); |
| | | | | | | | LCMS: ESI [M + H]⁺ = 541.1 |
| 480 | 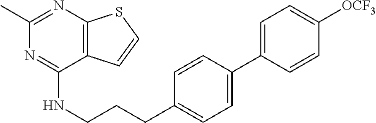 [SMILES: Cc1nc(NCCCc2ccc(-c3ccc(OC(F)(F)F)cc3)cc2)c2ccsc2n1] | 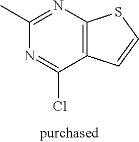 | A | Method D 1 d (60° C.) | 17% | 2-methyl-N-(3-(4′- (trifluoromethoxy)- [1,1′-biphenyl]-4- yl)propyl)thieno [2,3-d]pyrimidin- 4-amine | ¹H NMR (400 MHz, CHLOROFORM-d) δ 7.59 (d, J = 8.56 Hz, 2H), 7.51 (d, J = 7.79 Hz, 2H), 7.29 (d, J = 8.56 Hz, 2H), 7.33 (d, J = 8.17 Hz, 2H), 7.16 (d, J = 5.45 Hz, 1H), 6.99 (br. s., 1H), 3.69- 3.78 (m, 2H), 2.84 (t, J = 7.40 Hz, 2H), 2.65 (s, 3H), |
| | | | | | | | 2.12 (quin, J = 7.10 Hz, 2H); |
| | | | | | | | LCMS: ESI [M + H]⁺ = 444.1 |
| 481 | 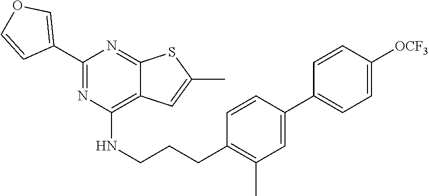 [SMILES: Cc1cc2c(NCCCc3ccc(-c4ccc(OC(F)(F)F)cc4)cc3C)nc(-c3ccoc3)nc2s1] | KH 6067 | B | Method D 1 d (90° C.) | 12% | 2-(furan-3-yl)-6- methyl-N-(3-(3- methyl-4′- (trifluoromethoxy)- [1,1′-biphenyl]-4- yl)propyl)thieno [2,3-d]pyrimidin- 4-amine | ¹H NMR (399 MHz, METHANOL-d4) δ 8.05 (s, 1H), 7.66 (d, J = 7.01 Hz, 2H), 7.47-7.53 (m, 1H), 7.23-7.41 (m, 5H), 7.02 (s, 1H), 6.95 (s, 1H), 3.70 (t, J = 6.42 Hz, 2H), 2.79 (t, J = 7.20 Hz, 2H), 2.53 (s, 3H), 2.35 (s, 3H), 1.97- 2.08 (m, 2H); LCMS: ESI [M + H]⁺ = 524.1 |
| 482 | 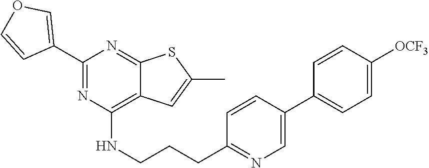 [SMILES: Cc1cc2c(NCCCc3ccc(-c4ccc(OC(F)(F)F)cc4)cn3)nc(-c3ccoc3)nc2s1] | KH 6067 | B | Method D 1 d (60° C.) | 23% | 2-(furan-3-yl)-6- methyl-N-(3-(5-(4- (trifluoromethoxy) phenyl)pyridin-2- yl)propyl)thieno [2,3-d]pyrimidin- 4-amine | ¹H NMR (400 MHz, CHLOROFORM-d) δ 8.76 (br. s., 1H), 8.19 (br. s., 1H), 7.79 (d, J = 8.17 Hz, 1H), 7.54-7.62 (m, J = 7.40 Hz, 2H), 7.46 (s, 1H), 7.31-7.38 (m, J = 8.18 Hz, 2H), 7.28- 7.31 (m, 1H), 7.07 (s, 1H), 6.77 (br. s., 1H), 5.80 (br. s., 1H), 3.78 (q, J = 5.71 Hz, |
| | | | | | | | 2H), 3.05 (t, J = 6.81 Hz, |
| | | | | | | | 2H), 2.51 (s, 3H), 2.17-2.30 |
| | | | | | | | (m, J = 6.81, 6.81 Hz, 2H); |
| | | | | | | | LCMS: ESI [M + H]⁺ = 511.1 |
| 483 | 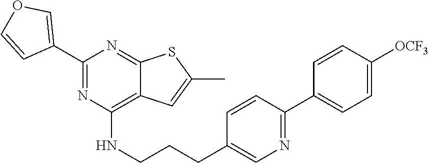 [SMILES: Cc1cc2c(NCCCc3ccc(-c4ccc(OC(F)(F)F)cc4)nc3)nc(-c3ccoc3)nc2s1] | KH 6067 | B | Method D 1 d (60° C.) | 39% | 2-(furan-3-yl)-6- methyl-N-(3-(6- (4- (trifluoromethoxy) phenyl)pyridin-3- yl)propyl)thieno [2,3-d]pyrimidin- 4-amine | ¹H NMR (400 MHz, CHLOROFORM-d) δ 8.58 (s, 1H), 8.19 (br. s., 1H), 7.97-8.05 (m, J = 8.17 Hz, 2H), 7.62 (s, 2H), 7.45 (s, 1H), 7.29-7.36 (m, J = 8.18 Hz, 2H), 7.05 (br. s., 1H), 6.66 (br. s., 1H), 4.98 (br. s., 1H), 3.76 (q, J = 6.62 Hz, 2H), 2.78-2.88 (m, 2H), 2.50 |
| | | | | | | | (s, 3H), 2.12 (quin, J = 6.72 |
| | | | | | | | Hz, 2H); LCMS: ESI |
| | | | | | | | [M + H]⁺ = 511.1 |
| 484 | 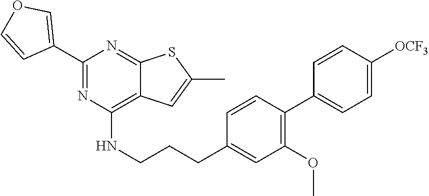 | KH 6067 | B | Method D 1 d (60° C.) | 41% | 2-(furan-3-yl)-N- (3-(2-methoxy-4′- (trifluoromethoxy)- [1,1′-biphenyl]-4- yl)propyl)-6- methylthieno[2,3- d]pyrimidin-4- amine | ¹H NMR (400 MHz, CHLOROFORM-d) δ 8.16 (br. s., 1H), 7.55 (d, J = 8.56 Hz, 2H), 7.46 (s, 1H), 7.22- 7.26 (m, J = 8.17 Hz, 3H), 7.05 (br. s., 1H), 6.92 (d, J = 7.40 Hz, 1H), 6.83 (br. s., 1H), 6.59 (br. s., 1H), 4.95 (br. s., 1H), 3.69-3.83 (m, 5H), 2.83 (t, J = 6.81 Hz, 2H), 2.50 (s, 3H), 2.05-2.19 (m, 2H); LCMS: ESI |
| | | | | | | | [M + H]⁺ = 540.1 |
| 485 | 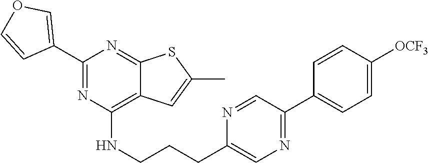 [SMILES: Cc1cc2c(NCCCc3cnc(-c4ccc(OC(F)(F)F)cc4)cn3)nc(-c3ccoc3)nc2s1] | KH 6067 | B | Method D 3 d (60° C.) | 67% | 2-(furan-3-yl)-6- methyl-N-(3-(5-(4- (trifluoromethoxy) phenyl)pyrazin-2- yl)propyl)thieno [2,3-d]pyrimidin- 4-amine | 1H NMR (400 MHz, CHLOROFORM-d) δ 8.93 (s, 1H), 8.53 (s, 1H), 8.19 (br. s., 1H), 7.99-8.07 (m, J = 8.56 Hz, 2H), 7.45 (s, 1H), 7.31-7.40 (m, J = 8.17 Hz, 2H), 7.06 (br. s., 1H), 6.72 (br. s., 1H), 5.33 (br. s., 1H), 3.80 (q, J = 6.23 Hz, 2H), 3.03 (t, J = 7.20 |
| | | | | | | | Hz, 2H), 2.52 (s, 3H), 2.19- |
| | | | | | | | 2.31 (m, 2H); LCMS: ESI |
| | | | | | | | [M + H]⁺ = 512.1 |
| 486 | 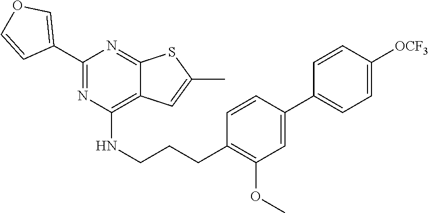 [SMILES: COc1cc(-c2ccc(OC(F)(F)F)cc2)ccc1CCCNc1nc(-c2ccoc2)nc2sc(C)cc12] | KH 6067 | B | Method D 1 d (60° C.) | 32% | 2-(furan-3-yl)-N- (3-(3-methoxy-4′- (trifluoromethoxy)- [1,1′-biphenyl]-4- yl)propyl)-6- methylthieno[2,3- d]pyrimidin-4- amine | ¹H NMR (400 MHz, CHLOROFORM-d) δ 8.16 (br. s., 1H), 7.56-7.64 (m, 2H), 7.45 (s, 1H), 7.30 (d, J = 7.79 Hz, 2H), 7.25 (d, J = 7.40 Hz, 1H), 7.11 (dd, J = 1.56, 7.79 Hz, 1H), 6.99- 7.07 (m, 2H), 6.61 (s, 1H), 5.07 (br. s., 1H), 3.88 (s, 3H), 3.71 (q, J = 6.62 Hz, 2H), 2.82 (t, J = 7.20 Hz, 2H), 2.49 (s, 3H), 2.06 (quin, J = 6.91 Hz, 2H); |
| | | | | | | | LCMS: ESI [M + H]⁺ = 540.1 |
| 487 | 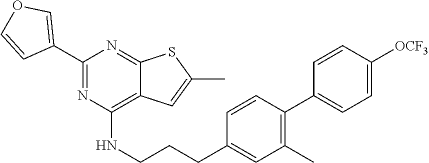 [SMILES: Cc1cc2c(NCCCc3ccc(-c4ccc(OC(F)(F)F)cc4)c(C)c3)nc(-c3ccoc3)nc2s1] | KH 6067 | B | Method D 1 d (60° C.), 1 d (90° C.) | 5% | 2-(furan-3-yl)-6- methyl-N-(3-(2- methyl-4′- (trifluoromethoxy)- [1,1′-biphenyl]-4- yl)propyl)thieno [2,3-d]pyrimidin- 4-amine | ¹H NMR (399 MHz, METHANOL-d4) δ 8.53 (s, 1H), 8.04 (s, 1H), 7.53 (t, J = 1.75 Hz, 1H), 7.33-7.40 (m, 2H), 7.28-7.33 (m, 2H), 7.09-7.14 (m, 2H), 7.02 (d, J = 1.56 Hz, 1H), 6.98 (d, J = 1.17 Hz, 1H), 3.68 (t, J = 7.01 Hz, 2H), 2.76 (t, J = 7.20 Hz, 2H), 2.55 (s, |
| | | | | | | | 3H), 2.17 (s, 3H), 2.09 (td, |
| | | | | | | | J = 7.35, 14.50 Hz, 2H); |
| | | | | | | | LCMS: ESI [M + H]⁺ = 524.2 |

### Example 7: Scheme 5 Aryl Chloride Functionalization

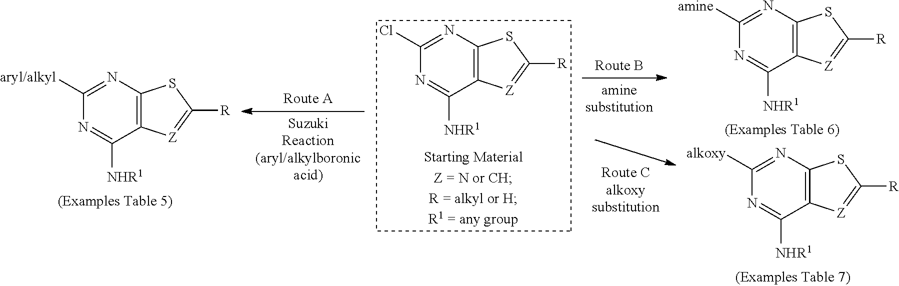

General Procedure for Scheme 5: Reaction conditions and compound data for specific examples for Routes A, B, and C are listed in Tables 5, 6, and 7, respectively. The starting material was either synthesized as detailed in the building blocks section or as examples described in Schemes 1-4.

Scheme 5 (Route A): Following the General Suzuki Protocol, the starting material (0.043 mmol) was reacted with the appropriate aryl(or alkyl)boronic acid (0.085 mmol) for the time and temperature specified, and the reaction was purified by prep HPLC or silica gel column chromatography, or a combination of the two, to give the pure product (Table 5).

TABLE 5
Examples from Scheme 5 (Route A)

| | | | Step 2 | Step | |
|---|---|---|---|---|---|
| Cmpd. | | Starting | Method/ | 2 | |
| No. | Structure | Material | Conditions | Yield | IUPAC |
| 488 | 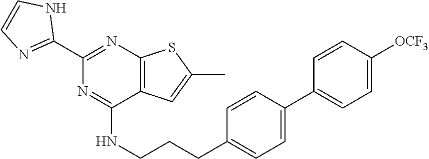 [SMILES: Cc1cc2c(NCCCc3ccc(-c4ccc(OC(F)(F)F)cc4)cc3)nc(-c3ncc[nH]3)nc2s1] | 433 | Cs₂CO₃ 1 h (180° C.) Microwave | 3% | 2-(1H-imidazol-2-yl)- 6-methyl-N-(3-(4′- (trifluoromethoxy)- [1,1′-bipheny]]-4- yl)propyl)thieno[2,3- d]pyrimidin-4-amine |
| 489 | 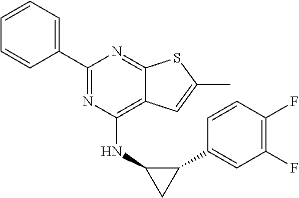 [SMILES: Cc1cc2c(N[C@@H]3C[C@H]3c3ccc(F)c(F)c3)nc(-c3ccccc3)nc2s1] | 222 | Cs₂CO₃ 3 h (80° C.) | 70% | N-((1R,2S)-2-(3,4- difluorophenyl) cyclopropyl)-6-methyl-2- phenylthieno[2,3- d]pyrimidin-4-amine |
| 490 | 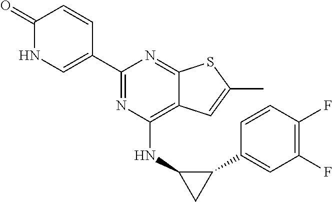 [SMILES: Cc1cc2c(N[C@@H]3C[C@H]3c3ccc(F)c(F)c3)nc(-c3ccc(=O)[nH]c3)nc2s1] | 222 | Cs₂CO₃ 1.5 h (80° C.) | 37% | 5-(4-(((1R,2S)-2-(3,4- difluorophenyl) cyclopropyl)amino)-6- methylthieno[2,3- d]pyrimidin-2- yl)pyridin-2(1H)-one |
| 491 | 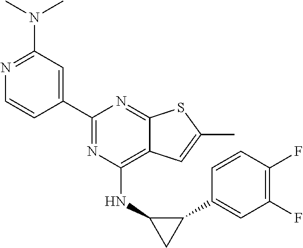 [SMILES: Cc1cc2c(N[C@@H]3C[C@H]3c3ccc(F)c(F)c3)nc(-c3ccnc(N(C)C)c3)nc2s1] | 222 | Cs₂CO₃ 1.5 h (80° C.) | 70% | N-((1R,2S)-2-(3,4- difluorophenyl) cyclopropyl)-2-(2- (dimethylamino) pyridin-4-yl)-6- methylthieno[2,3- d]pyrimidin-4-amine |
| 492 | 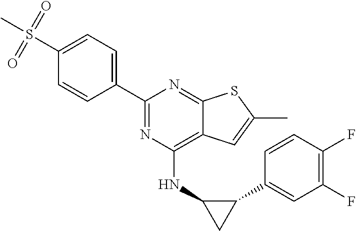 [SMILES: Cc1cc2c(N[C@@H]3C[C@H]3c3ccc(F)c(F)c3)nc(-c3ccc(S(C)(=O)=O)cc3)nc2s1] | 222 | Cs₂CO₃ 1.5 h (80° C.) | 56% | N-((1R,2S)-2-(3,4- difluorophenyl) cyclopropyl)-6- methyl-2-(4- (methylsulfonyl)phenyl) thieno[2,3- d]pyrimidin-4-amine |
| 493 | 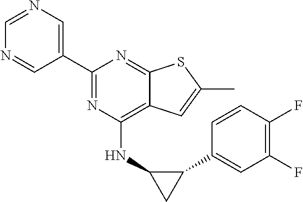 [SMILES: Cc1cc2c(N[C@@H]3C[C@H]3c3ccc(F)c(F)c3)nc(-c3cncnc3)nc2s1] | 222 | Cs₂CO₃ 2 h (80° C.) | 69% | N-((1R,2S)-2-(3,4- difluorophenyl) cyclopropyl)-6-methyl-2- (pyrimidin-5- yl)thieno[2,3- d]pyrimidin-4-amine |
| 494 | 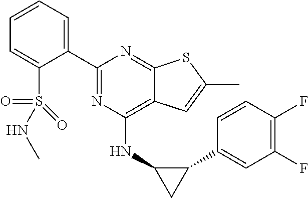 [SMILES: CNS(=O)(=O)c1ccccc1-c1nc(N[C@@H]2C[C@H]2c2ccc(F)c(F)c2)c2cc(C)sc2n1] | 222 | Cs₂CO₃ 2 h (80° C.) | 55% | 2-(4-(((1R,2S)-2-(3,4- difluorophenyl) cyclopropyl)amino)-6- methylthieno[2,3- d]pyrimidin-2-yl)-N- methylbenzenesulfonamide |
| 495 | 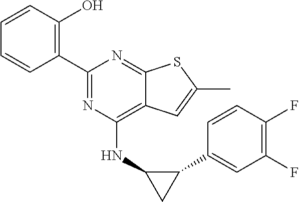 [SMILES: Cc1cc2c(N[C@@H]3C[C@H]3c3ccc(F)c(F)c3)nc(-c3ccccc3O)nc2s1] | 222 | Cs₂CO₃ 4 h (80° C.) | 25% | 2-(4-(((1R,2S)-2-(3,4- difluorophenyl) cyclopropyl)amino)-6- methylthieno[2,3- d]pyrimidin-2- yl)phenol |
| 496 | 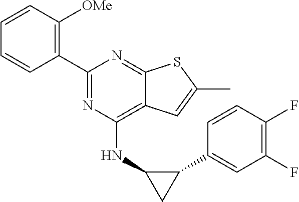 [SMILES: COc1ccccc1-c1nc(N[C@@H]2C[C@H]2c2ccc(F)c(F)c2)c2cc(C)sc2n1] | 222 | Cs₂CO₃ 4 h (80° C.) | 66% | N-((1R,2S)-2-(3,4- difluorophenyl) cyclopropyl)-2-(2- methoxyphenyl)-6- methylthieno[2,3- d]pyrimidin-4-amine |
| 497 | 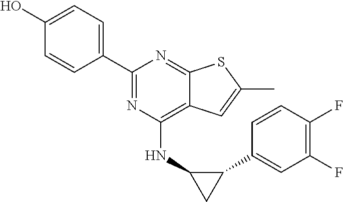 [SMILES: Cc1cc2c(N[C@@H]3C[C@H]3c3ccc(F)c(F)c3)nc(-c3ccc(O)cc3)nc2s1] | 222 | Cs₂CO₃ 1 h (80° C.), 2 h (100° C.) | 70% | 4-(4-(((1R,2S)-2-(3,4- difluorophenyl) cyclopropyl)amino)-6- methylthieno[2,3- d]pyrimidin-2- yl)phenol |
| 498 | 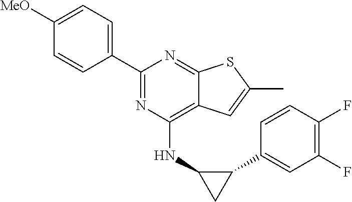 [SMILES: COc1ccc(-c2nc(N[C@@H]3C[C@H]3c3ccc(F)c(F)c3)c3cc(C)sc3n2)cc1] | 222 | Cs₂CO₃ 2 h (70° C.), 1 h (80° C.) | 10% | N-((1R,2S)-2-(3,4- difluorophenyl) cyclopropyl)-2-(4- methoxyphenyl)-6- methylthieno[2,3- d]pyrimidin-4-amine |
| 499 | 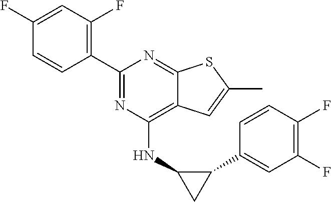 [SMILES: Cc1cc2c(N[C@@H]3C[C@H]3c3ccc(F)c(F)c3)nc(-c3ccc(F)cc3F)nc2s1] | 222 | Cs₂CO₃ 3 h (70° C.) | 49% | 2-(2,4- difluorophenyl)-N- ((1R,2S)-2-(3,4- difluorophenyl) cyclopropyl)-6- methylthieno[2,3- d]pyrimidin-4-amine |
| 500 | 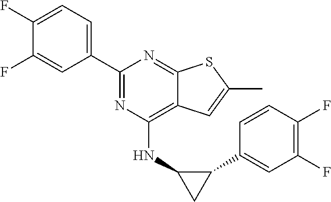 [SMILES: Cc1cc2c(N[C@@H]3C[C@H]3c3ccc(F)c(F)c3)nc(-c3ccc(F)c(F)c3)nc2s1] | 222 | Cs₂CO₃ 1.5 h (80° C.) | 59% | 2-(3,4- difluorophenyl)-N- ((1R,2S)-2-(3,4- difluorophenyl) cyclopropyl)-6- methylthieno[2,3- d]pyrimidin-4-amine |
| 501 | 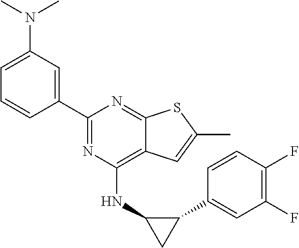 [SMILES: Cc1cc2c(N[C@@H]3C[C@H]3c3ccc(F)c(F)c3)nc(-c3cccc(N(C)C)c3)nc2s1] | 222 | Cs₂CO₃ 3.5 h (80° C.) | 74% | N-((1R,2S)-2-(3,4- difluorophenyl) cyclopropyl)-2-(3- (dimethylamino)phenyl)- 6-methylthieno[2,3- d]pyrimidin-4-amine |
| 502 | 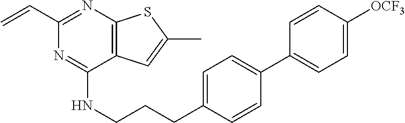 [SMILES: C=Cc1nc(NCCCc2ccc(-c3ccc(OC(F)(F)F)cc3)cc2)c2cc(C)sc2n1] | 433 | K₂CO₃ 21 h (100° C.) | 67% | 2-ethenyl-6-methyl-N- (3-[4′- (trifluoromethoxy)- [1,1′-biphenyl]-4- yl]propyl)thieno[2,3- d]pyrimidin-4-amine |

Examples from Scheme 5 (Route A)

| Cmpd. | |
|---|---|
| No. | Compound Data |
| 488 | ¹H NMR (399 MHz, METHANOL-d4) δ |
| | 8.53 (s, 1H), 7.83 (s, 1H), 7.68 (d, J = 8.56 |
| | Hz, 2H), 7.53 (d, J = 8.17 Hz, 2H), 7.33 (d, |
| | J = 8.17 Hz, 4H), 7.05 (s, 1H), 7.01 (s, 1H), |
| | 3.69 (t, J = 7.01 Hz, 2H), 2.82 (t, J = 7.20 |
| | Hz, 2H), 2.53 (s, 3H), 2.11 (quin, J = 7.20 |
| | Hz, 1H); LCMS: ESI [M + H]⁺ = 510.1 |
| 489 | ¹H NMR (400 MHz, METHANOL-d4) δ |
| | 8.15 (d, J = 7.43 Hz, 2H), 7.32 − 7.44 (m, |
| | 1H), 7.23 − 7.30 (m, 2H), 7.10 − 7.20 (m, |
| | 2H), 6.98 − 7.07 (m, 2H), 3.03 − 3.14 (m, |
| | 1H), 2.54 (s, 3H), 2.04 (ddd, J = 2.93, 6.26, |
| | 9.19 Hz, 1H), 1.36 − 1.56 (m, 2H); LCMS: |
| | ESI [M + H]⁺ = 394.1 |
| 490 | ¹H NMR (400 MHz, METHANOL-d4) δ |
| | 8.19 − 8.30 (m, 2H), 7.06 − 7.24 (m, 2H), |
| | 6.97 − 7.05 (m, 2H), 6.39 (d, J = 10.17 Hz, |
| | 1H), 3.01 − 3.10 (m, 1H), 2.53 (s, 3H), 2.03 |
| | (ddd, J = 2.93, 6.36, 9.49 Hz, 1H), 1.34 − |
| | 1.51 (m, 2H); LCMS: ESI [M + H]⁺ = 411.1 |
| 491 | ¹H NMR (400 MHz, CHLOROFORM-d) δ |
| | 8.20 (d, J = 5.09 Hz, 1H), 7.50 (s, 1H), 7.42 |
| | (d, J = 5.48 Hz, 1H), 7.06 − 7.17 (m, 1H), |
| | 6.89 − 7.04 (m, 2H), 6.80 (s, 1H), 5.48 (br. |
| | s., 1H), 3.14 − 3.22 (m, 1H), 3.07 (s, 6H), |
| | 2.55 (s, 3H), 2.13 (ddd, J = 2.93, 6.26, 9.19 |
| | Hz, 1H), 1.33 − 1.46 (m, 2H); LCMS: ESI |
| | [M + H]⁺ = 438.1 |
| 492 | ¹H NMR (400 MHz, CHLOROFORM-d) δ |
| | 8.50 − 8.56 (m, 2H), 7.90 − 7.96 (m, 2H), |
| | 7.11 − 7.19 (m, 1H), 7.02 − 7.09 (m, 1H), |
| | 6.93 − 7.00 (m, 1H), 6.82 (s, 1H), 5.52 (br. |
| | s., 1H), 3.11 − 3.17 (m, 1H), 3.09 (d, J = |
| | 1.57 Hz, 3H), 2.58 (s, 3H), 2.15 (t, J = 6.46 |
| | Hz, 1H), 1.43 (t, J = 6.85 Hz, 2H); LCMS: |
| | ESI [M + H]⁺ = 472.0 |
| 493 | ¹H NMR (400 MHz, CHLOROFORM-d) δ |
| | 9.58 (s, 2H), 9.25 (s, 1H), 7.10 − 7.20 (m, |
| | 1H), 6.92 − 7.02 (m, 2H), 6.82 (d, J = 1.17 |
| | Hz, 1H), 5.57 (br. s., 1H), 3.16 (tdd, J = |
| | 2.69, 4.79, 7.04 Hz, 1H), 2.57 (s, 3H), 2.12 − |
| | 2.20 (m, 1H), 1.39 − 1.46 (m, 2H); LCMS: |
| | ESI [M + H]⁺ = 396.1 |
| 494 | ¹H NMR (400 MHz, CHLOROFORM-d) δ |
| | 8.10 (d, J = 7.43 Hz, 1H), 7.84 − 7.96 (m, |
| | 2H), 7.50 − 7.63 (m, 2H), 6.85 − 6.95 (m, |
| | 1H), 6.74 − 6.82 (m, 1H), 6.63 (br. s., 1H), |
| | 6.55 (s, 1H), 6.27 (br. s., 1H), 2.71 − 2.77 |
| | (m, 3H), 2.62 (br. s., 1H), 2.40 (s, 3H), 1.95 |
| | (d, J = 3.52 Hz, 1H), 0.99 − 1.08 (m, 1H), |
| | 0.79 (d, J = 5.87 Hz, 1H); LCMS: ESI |
| | [M + H]⁺ = 487.1 |
| 495 | ¹H NMR (400 MHz, CHLOROFORM-d) δ |
| | 13.50 (s, 1H), 8.31 (d, J = 7.83 Hz, 1H), |
| | 7.29 − 7.36 (m, 1H), 7.08 − 7.16 (m, 1H), |
| | 6.90 − 7.04 (m, 3H), 6.83 (t, J = 7.63 Hz, |
| | 1H), 6.77 (s, 1H), 5.53 (br. s., 1H), 3.10 (d, |
| | J = 2.35 Hz, 1H), 2.55 (s, 3H), 2.13 − 2.22 |
| | (m, 1H), 1.35 − 1.52 (m, 2H); LCMS: ESI |
| | [M + H]⁺ = 410.1 |
| 496 | ¹H NMR (400 MHz, METHANOL-d4) δ |
| | 7.40 − 7.46 (m, 1H), 7.27 − 7.34 (m, 1H), |
| | 7.10 (dd, J = 7.83, 11.35 Hz, 1H), 6.90 − |
| | 7.01 (m, 4H), 6.86 (t, J = 7.43 Hz, 1H), |
| | 3.66 (d, J = 1.17 Hz, 3H), 2.87 − 2.94 (m, |
| | 1H), 2.45 (s, 3H), 1.98 (t, J = 9.00 Hz, 1H), |
| | 1.28 (td, J = 5.14, 9.68 Hz, 1H), 1.16 − 1.23 |
| | (m, 1H); LCMS: ESI [M + H]⁺ = 424.1 |
| 497 | ¹H NMR (400 MHz, METHANOL-d4) δ |
| | 7.92 (d, J = 8.61 Hz, 2H), 7.00 − 7.14 (m, |
| | 2H), 6.93 (s, 2H), 6.59 (d, J = 8.61 Hz, 2H), |
| | 2.91 − 3.01 (m, 1H), 2.44 (s, 3H), 1.94 (br. |
| | s., 1H), 1.37 (td, J = 5.14, 9.68 Hz, 1H), |
| | 1.28 (q, J = 6.39 Hz, 1H); LCMS: ESI |
| | [M + H]⁺ = 410.1 |
| 498 | ¹H NMR (400 MHz, CHLOROFORM-d) δ |
| | 8.28 (d, J = 8.61 Hz, 2H), 7.03 − 7.20 (m, |
| | 2H), 6.99 (br. s., 1H), 6.89 (d, J = 8.61 Hz, |
| | 2H), 6.76 (s, 1H), 5.37 (br. s., 1H), 3.87 (s, |
| | 3H), 3.13 (br. s., 1H), 2.54 (s, 3H), 2.14 (br. |
| | s., 1H), 1.41 (t, J = 7.04 Hz, 2H); LCMS: |
| | ESI [M + H]⁺ = 424.1 |
| 499 | ¹H NMR (400 MHz, CHLOROFORM-d) δ |
| | 7.94 − 8.05 (m, 1H), 7.02 − 7.16 (m, 2H), |
| | 6.85 − 6.99 (m, 3H), 6.80 (s, 1H), 5.51 (br. |
| | s., 1H), 3.09 (br. s., 1H), 2.54 (s, 3H), 2.09 − |
| | 2.19 (m, 1H), 1.32 − 1.41 (m, 2H); LCMS: |
| | ESI [M + H]⁺ = 430.0 |
| 500 | ¹H NMR (400 MHz, CHLOROFORM-d) δ |
| | 8.09 (ddq, J = 8.9, 6.6, 3.8, 3.0 Hz, 2H), |
| | 7.12 (tt, J = 11.4, 7.8 Hz, 2H), 6.99 (ddd, J = |
| | 24.5, 11.3, 5.5 Hz, 2H), 6.79 − 6.74 (m, |
| | 1H), 5.44 (s, 1H), 3.09 (td, J = 6.1, 3.2 Hz, |
| | 1H), 2.56 − 2.52 (m, 3H), 2.11 (t, J = 8.0 |
| | Hz, 1H), 1.40 (ddd, J = 7.9, 5.9, 2.2 Hz, |
| | 2H); LCMS: ESI [M + H]⁺ = 430.1 |
| 501 | ¹H NMR (400 MHz, CHLOROFORM-d) δ |
| | 7.79 (d, J = 2.3 Hz, 1H), 7.70 (d, J = 7.6 |
| | Hz, 1H), 7.26 − 7.21 (m, 1H), 7.14 − 7.02 |
| | (m, 1H), 7.02 − 6.89 (m, 2H), 6.81 (dt, J = |
| | 8.5, 2.4 Hz, 1H), 6.75 (q, J = 1.4 Hz, 1H), |
| | 5.46 (s, 1H), 3.17 (dt, J = 7.6, 3.8 Hz, 1H), |
| | 2.91 (t, J = 1.5 Hz, 6H), 2.54 − 2.44 (m, |
| | 3H), 2.10 (q, J = 5.8, 4.8 Hz, 1H), 1.42 − |
| | 1.27 (m, 2H); LCMS: ESI [M + H]⁺ = 437.2 |
| 502 | ¹H NMR (399 MHz, ACETONITRILE-d3) |
| | δ 7.70 (d, J = 7.40 Hz, 2H), 7.55 (d, J = |
| | 6.62 Hz, 2H), 7.35 (dd, J = 8.56, 11.29 Hz, |
| | 4H), 6.93 (s, 1H), 6.66 (dd, J = 10.51, 17.13 |
| | Hz, 1H), 6.41 (d, J = 17.13 Hz, 1H), 6.13 |
| | (br. s., 1H), 5.54 (d, J = 10.12 Hz, 1H), 3.63 |
| | (q, J = 6.62 Hz, 2H), 2.78 (t, J = 7.40 Hz, |
| | 2H), 2.51 (s, 3H), 2.02 (t, J = 7.20 Hz, 2H); |
| | LCMS: ESI [M + H]⁺ = 470.1 |

Scheme 5 (Route B): The specified starting material (0.06 mmol) and the appropriate amine (4 eqv. unless otherwise noted) were dissolved in the specified solvent(s) (1.0 mL) and if listed, DIPEA (6 eqv.), and the mixture was reacted under the conditions detailed in Table 6. Upon completion, the reaction was concentrated in vacuo and then purified by prep HPLC to give the desired product (Table 6).

TABLE 6
Examples from Scheme 5 (Route B)

| | |
|---|---|
| Cmpd. | Starting |

| No. | Structure | Material | amine | Solvent | Conditions |
|---|---|---|---|---|---|
| 503 | 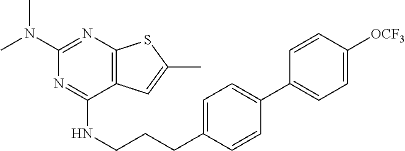 [SMILES: Cc1cc2c(NCCCc3ccc(-c4ccc(OC(F)(F)F)cc4)cc3)nc(N(C)C)nc2s1] | 463 | [2M] NHMe₂ iPrOH (25 eqv) | Neat | 16 h (60° C.) oil bath |
| 504 | 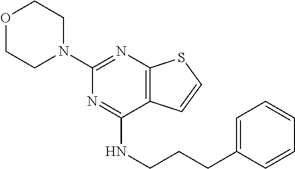 [SMILES: c1ccc(CCCNc2nc(N3CCOCC3)nc3sccc23)cc1] | 218 | 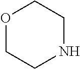 | DMSO/ DIPEA | 1 d (90° C.) oil bath |
| 505 | 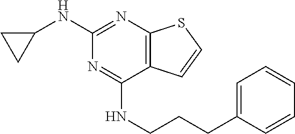 [SMILES: c1ccc(CCCNc2nc(NC3CC3)nc3sccc23)cc1] | 218 | 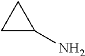 | Dioxane | 2 h (200° C.) microwave |
| 506 | 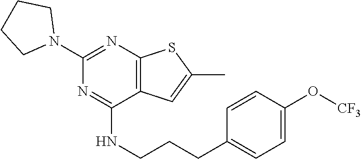 [SMILES: Cc1cc2c(NCCCc3ccc(OC(F)(F)F)cc3)nc(N3CCCC3)nc2s1] | 223 | 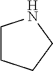 | n-butanol | 30 min (150° C.) microwave |
| 507 | 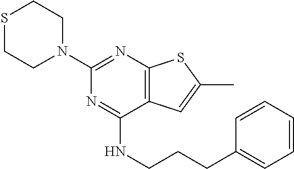 [SMILES: Cc1cc2c(NCCCc3ccccc3)nc(N3CCSCC3)nc2s1] | 224 | 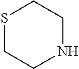 | n-butanol | 30 min (150° C.) Microwave |
| 508 | 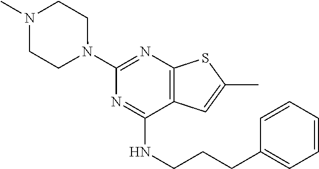 [SMILES: Cc1cc2c(NCCCc3ccccc3)nc(N3CCN(C)CC3)nc2s1] | 224 | 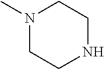 | n-butanol | 30 min (150° C.) Microwave |
| 509 | 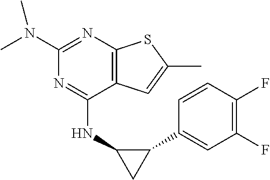 [SMILES: Cc1cc2c(N[C@@H]3C[C@H]3c3ccc(F)c(F)c3)nc(N(C)C)nc2s1] | 222 | 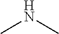 | n-butanol | 1 h (160° C.) Microwave |
| 510 | 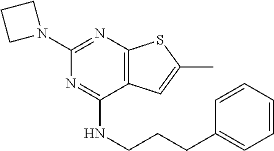 [SMILES: Cc1cc2c(NCCCc3ccccc3)nc(N3CCC3)nc2s1] | 224 | 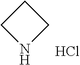 | n- butanol/ DIPEA | 30 min (150° C.) Microwave |
| 511 | 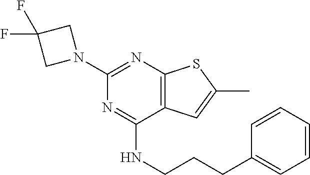 [SMILES: Cc1cc2c(NCCCc3ccccc3)nc(N3CC(F)(F)C3)nc2s1] | 224 | 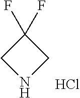 | n- butanol/ DIPEA | 30 min (150° C.) Microwave |
| 512 | 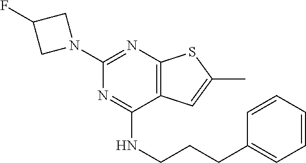 [SMILES: Cc1cc2c(NCCCc3ccccc3)nc(N3CC(F)C3)nc2s1] | 224 | 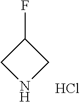 | n-butanol DIPEA | 30 min (150° C.) Microwave |
| 513 | 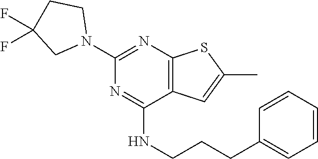 [SMILES: Cc1cc2c(NCCCc3ccccc3)nc(N3CCC(F)(F)C3)nc2s1] | 224 | 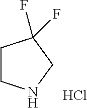 | n-butanol DIPEA | 30 min (150° C.) Microwave |
| 514 | 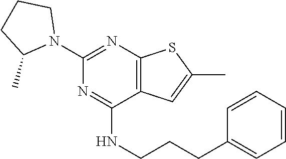 [SMILES: Cc1cc2c(NCCCc3ccccc3)nc(N3CCC[C@H]3C)nc2s1] | 224 | 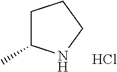 | n-butanol DIPEA | 30 min (150° C.) Microwave |
| 515 | 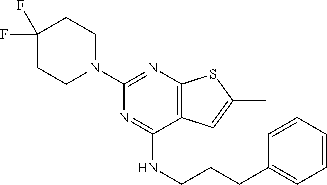 [SMILES: Cc1cc2c(NCCCc3ccccc3)nc(N3CCC(F)(F)CC3)nc2s1] | 224 | 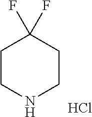 | n-butanol DIPEA | 30 min (150° C.) Microwave |
| 516 | 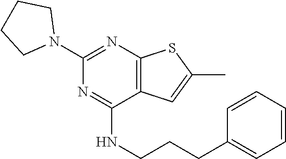 [SMILES: Cc1cc2c(NCCCc3ccccc3)nc(N3CCCC3)nc2s1] | 224 | 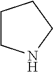 | n-butanol | 30 min (150° C.) Microwave |
| 517 | 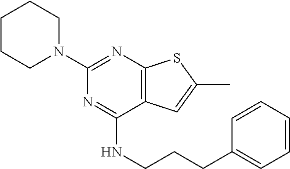 [SMILES: Cc1cc2c(NCCCc3ccccc3)nc(N3CCCCC3)nc2s1] | 224 | 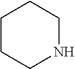 | n-butanol | 1 h (150° C.) Microwave |
| 518 | 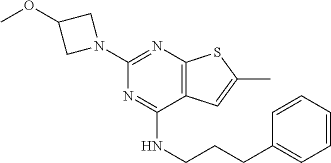 [SMILES: COC1CN(c2nc(NCCCc3ccccc3)c3cc(C)sc3n2)C1] | 224 | 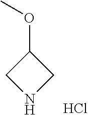 | n-butanol DIPEA | 1 h (150° C.) Microwave |
| 519 | 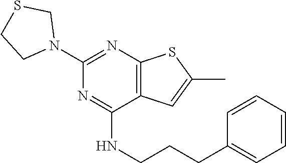 [SMILES: Cc1cc2c(NCCCc3ccccc3)nc(N3CCSC3)nc2s1] | 224 | 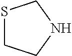 | n-butanol | 2 h (160° C.) Microwave |
| 520 | 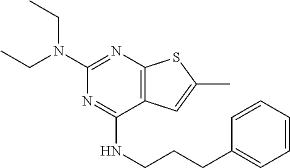 [SMILES: CCN(CC)c1nc(NCCCc2ccccc2)c2cc(C)sc2n1] | 224 | 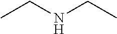 | n-butanol | 1 h (150° C.) Microwave |
| 521 | 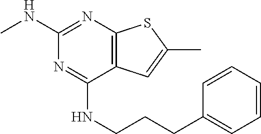 [SMILES: CNc1nc(NCCCc2ccccc2)c2cc(C)sc2n1] | 224 | 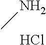 | n-butanol DIPEA | 1 h (150° C.) Microwave |
| 522 | 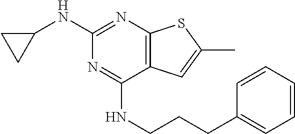 [SMILES: Cc1cc2c(NCCCc3ccccc3)nc(NC3CC3)nc2s1] | 224 | 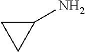 | n-butanol | 2 h (200° C.) Microwave |
| 523 | 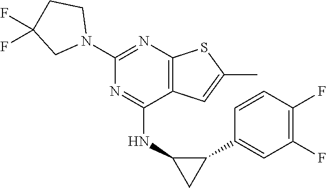 [SMILES: Cc1cc2c(N[C@@H]3C[C@H]3c3ccc(F)c(F)c3)nc(N3CCC(F)(F)C3)nc2s1] | 222 | 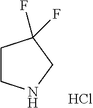 | n-butanol DIPEA | 1 h (150° C.) Microwave |
| 524 | 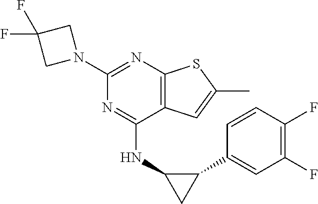 [SMILES: Cc1cc2c(N[C@@H]3C[C@H]3c3ccc(F)c(F)c3)nc(N3CC(F)(F)C3)nc2s1] | 222 | 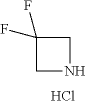 | n-butanol DIPEA | 1 h (150° C.) Microwave |
| 525 | 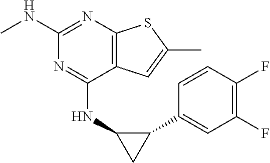 [SMILES: CNc1nc(N[C@@H]2C[C@H]2c2ccc(F)c(F)c2)c2cc(C)sc2n1] | 222 | —NH₂ (2M in THF) | n-butanol DIPEA | 3 d (90° C.) Oil bath |
| 526 | 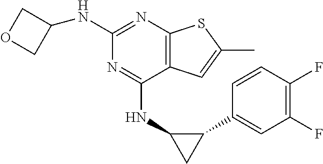 [SMILES: Cc1cc2c(N[C@@H]3C[C@H]3c3ccc(F)c(F)c3)nc(NC3COC3)nc2s1] | 222 | 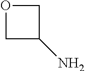 | n-butanol DIPEA | 3 d (90° C.) Oil bath |
| 527 | 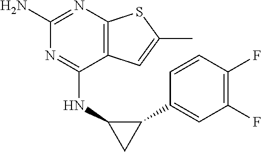 [SMILES: Cc1cc2c(N[C@@H]3C[C@H]3c3ccc(F)c(F)c3)nc(N)nc2s1] | 222 | 30% NH₄OH (aq) | Dioxane | 1 h (90° C.) Microwave |
| 528 | 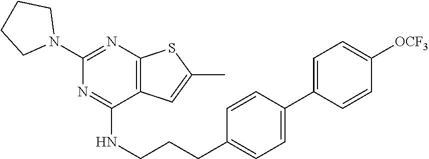 [SMILES: Cc1cc2c(NCCCc3ccc(-c4ccc(OC(F)(F)F)cc4)cc3)nc(N3CCCC3)nc2s1] | 433 | 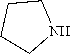 | n-butanol | 30 min (160° C.) Microwave |
| 529 | 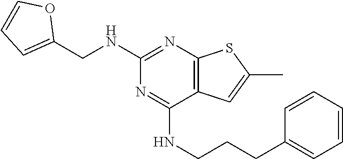 [SMILES: Cc1cc2c(NCCCc3ccccc3)nc(NCc3ccco3)nc2s1] | 224 | 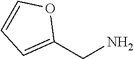 | DMSO DIPEA | 1 h (150° C.) Microwave |
| 530 | 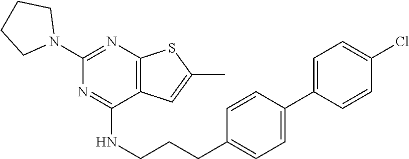 [SMILES: Cc1cc2c(NCCCc3ccc(-c4ccc(Cl)cc4)cc3)nc(N3CCCC3)nc2s1] | 435 | 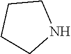 | n-butanol | 30 min (150° C.) Microwave |
| 531 | 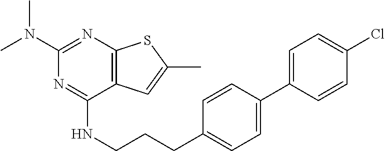 [SMILES: Cc1cc2c(NCCCc3ccc(-c4ccc(Cl)cc4)cc3)nc(N(C)C)nc2s1] | 435 | 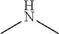 | n-butanol | 30 min (160° C.) Microwave |
| 532 | 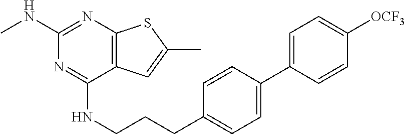 [SMILES: CNc1nc(NCCCc2ccc(-c3ccc(OC(F)(F)F)cc3)cc2)c2cc(C)sc2n1] | 433 | NH₂Me (2M in THF) | n-butanol | 30 min (180° C.) Microwave |
| 533 | 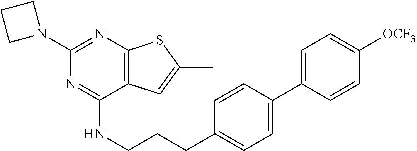 [SMILES: Cc1cc2c(NCCCc3ccc(-c4ccc(OC(F)(F)F)cc4)cc3)nc(N3CCC3)nc2s1] | 433 | 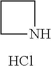 | n-butanol | 30 min (180° C.) Microwave |
| 534 | 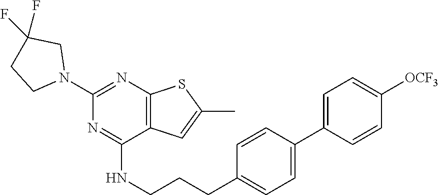 [SMILES: Cc1cc2c(NCCCc3ccc(-c4ccc(OC(F)(F)F)cc4)cc3)nc(N3CCC(F)(F)C3)nc2s1] | 433 | 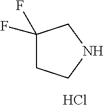 | n-butanol | 30 min (180° C.) Microwave |
| 535 | 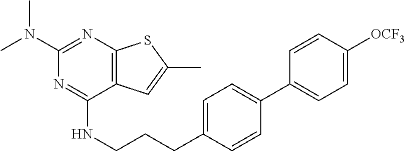 [SMILES: Cc1cc2c(NCCCc3ccc(-c4ccc(OC(F)(F)F)cc4)cc3)nc(N(C)C)nc2s1] | 433 | NHMe₂ [2M] in iPrOH (5 eqv) | H₂O/ DMF (1:2) | 30 min (190° C.) Microwave |
| 536 | 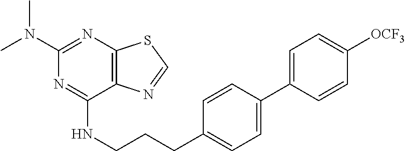 [SMILES: CN(C)c1nc(NCCCc2ccc(-c3ccc(OC(F)(F)F)cc3)cc2)c2ncsc2n1] | 468 | 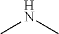 | n-butanol | 45 min (160° C.) Microwave |
| 537 | 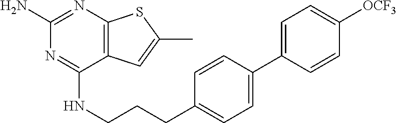 [SMILES: Cc1cc2c(NCCCc3ccc(-c4ccc(OC(F)(F)F)cc4)cc3)nc(N)nc2s1] | 433 | 30% NH₄OH (aq) | Dioxane | 2 h (150° C.) Microwave |
| 538 | 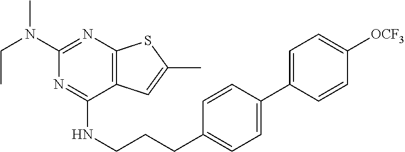 [SMILES: CCN(C)c1nc(NCCCc2ccc(-c3ccc(OC(F)(F)F)cc3)cc2)c2cc(C)sc2n1] | 433 | 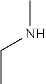 | Dioxane | 1 h (160° C.) Microwave |
| 539 | 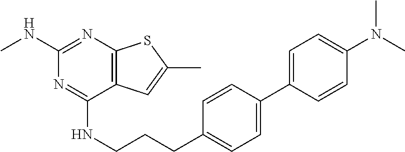 | 466 | —NH₂ (2M THF) | Dioxane | 2 h (190° C.) Microwave |
| 540 | 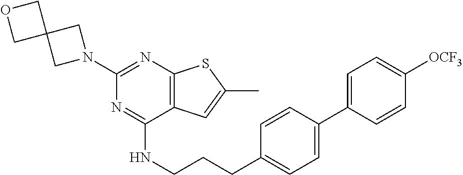 [SMILES: Cc1cc2c(NCCCc3ccc(-c4ccc(OC(F)(F)F)cc4)cc3)nc(N3CC4(COC4)C3)nc2s1] | 433 | 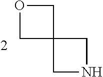 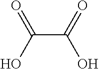 | Aceto- nitrile, DIPEA | 3 d (130° C.) oil bath |
| 541 | 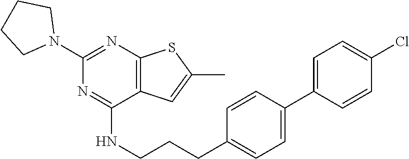 [SMILES: Cc1cc2c(NCCCc3ccc(-c4ccc(Cl)cc4)cc3)nc(N3CCCC3)nc2s1] | 435 | 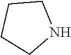 | n-butanol | 30 min (150° C.) Microwave |

Examples from Scheme 5 (Route B)

| Cmpd. | | Starting | | | |
|---|---|---|---|---|---|
| No. | Structure | Material | amine | Solvent | Conditions |
| 503 | | | 99% | N2,N5,5- | ¹H NMR (400 MHz, |
| | | | | trimethyl-N7-(3- | METHANOL-d4) δ 7.61 − |
| | | | | [4′- | 7.72 (m, 2H), 7.44 − 7.54 (m, |
| | | | | (trifluoromethoxy)- | 2H), 7.22 − 7.37 (m, 4H), 3.57 |
| | | | | [1,1′- | (t, J = 6.60 Hz, 2H), 3.01 − |
| | | | | biphenyl]-4- | 3.13 (m, 6H), 2.75 (t, J = 7.27 |
| | | | | yl]propyl)- | Hz, 2H), 2.58 − 2.65 (m, 3H), |
| | | | | [1,3]thiazolo [5,4- | 2.01 (t, J = 7.09 Hz, 2H); |
| | | | | d]pyrimidine-5,7- | LCMS: ESI [M + H]⁺ = 488.1 |
| | | | | diamine | |
| 504 | | | 29% | 2-morpholino-N- | ¹H NMR (400 MHz, |
| | | | | (3- | CHLOROFORM-d) δ 7.28 − |
| | | | | phenylpropyl) | 7.34 (m, 2H), 7.19 − 7.25 (m, |
| | | | | thieno[2,3- | 3H), 6.81 (s, 2H), 3.76 (s, 8H), |
| | | | | d]pyrimidin-4- | 3.61 (q, J = 6.91 Hz, 2H), 2.75 |
| | | | | amine | (t, J = 7.24 Hz, 2H), 1.98 − |
| | | | | | 2.07 (m, J = 7.04, 7.04 Hz, |
| | | | | | 2H); LCMS: ESI [M + H]⁺ = |
| | | | | | 355.1 |
| 505 | | | 6% | N2-cyclopropyl- | ¹H NMR (400 MHz, |
| | | | | N4-(3- | CHLOROFORM-d) δ 7.28 − |
| | | | | phenylpropyl) | 7.33 (m, 2H), 7.23 (d, J = 5.87 |
| | | | | thieno[2,3- | Hz, 3H), 6.82 (s, 2H), 3.62 (q, |
| | | | | d]pyrimidine-2,4- | J = 6.65 Hz, 2H), 2.75 (t, J = |
| | | | | diamine | 7.43 Hz, 2H), 2.04 (quin, J = |
| | | | | | 7.24 Hz, 2H), 0.76 − 0.81 (m, J = |
| | | | | | 5.48 Hz, 2H), 0.53 − 0.59 |
| | | | | | (m, 2H); LCMS: ESI |
| | | | | | [M + H]⁺ = 325.1 |
| 506 | | | 68% | 6-methyl-2- | ¹H NMR (400 MHz, |
| | | | | (pyrrolidin-1-yl)- | CHLOROFORM-d) δ 7.19 − |
| | | | | N-(3-(4- | 7.25 (m, J = 8.22 Hz, 2H), |
| | | | | (trifluoromethoxy) | 7.11 − 7.16 (m, J = 8.22 Hz, |
| | | | | phenyl)propyl)t | 2H), 6.47 (s, 1H), 4.72 − 4.78 |
| | | | | hieno[2,3- | (m, 1H), 3.53 − 3.63 (m, 6H), |
| | | | | d]pyrimidin-4- | 2.73 (t, J = 7.43 Hz, 2H), 2.44 |
| | | | | amine | (s, 3H), 1.97 − 2.04 (m, 2H), |
| | | | | | 1.92 − 1.97 (m, 4H); LCMS: |
| | | | | | ESI [M + H]⁺ = 437.2 |
| 507 | | | 16% | 6-methyl-N-(3- | ¹H NMR (400 MHz, |
| | | | | phenylpropyl)-2- | CHLOROFORM-d) δ 7.28 − |
| | | | | thiomorpholinothieno | 7.34 (m, 2H), 7.19 − 7.25 (m, |
| | | | | [2,3- | 3H), 6.45 (s, 1H), 4.76 (br. s., |
| | | | | d]pyrimidin-4- | 1H), 4.08 − 4.14 (m, J = 4.30 |
| | | | | amine | Hz, 4H), 3.56 (q, J = 6.26 Hz, |
| | | | | | 2H), 2.74 (t, J = 7.24 Hz, 2H), |
| | | | | | 2.60 − 2.66 (m, J = 3.91 Hz, |
| | | | | | 4H), 2.45 (s, 3H), 2.00 (quin, |
| | | | | | J = 7.24 Hz, 2H); LCMS: ESI |
| | | | | | [M + H]⁺ = 385.1 |
| 508 | | | 27% | 6-methyl-2-(4- | ¹H NMR (400 MHz, |
| | | | | methylpiperazin- | CHLOROFORM-d) δ 7.28 − |
| | | | | 1-yl)-N-(3- | 7.34 (m, 2H), 7.18 − 7.25 (m, |
| | | | | phenylpropyl) | J = 6.65 Hz, 3H), 6.44 (s, 1H), |
| | | | | thieno[2,3- | 4.70 − 4.77 (m, J = 5.67, 5.67 |
| | | | | d]pyrimidin-4- | Hz, 1H), 3.80 (br. s., 4H), 3.58 |
| | | | | amine | (q, J = 6.39 Hz, 2H), 2.74 (t, |
| | | | | | J = 7.43 Hz, 2H), 2.43 − 2.49 |
| | | | | | (m, 6H), 2.34 (s, 3H), 2.00 |
| | | | | | (quin, J = 7.14 Hz, 2H); |
| | | | | | LCMS: ESI [M + H]⁺ = 382.2 |
| 509 | | | 10% | N4-((1R,2S)-2- | ¹H NMR (400 MHz, |
| | | | | (3,4- | CHLOROFORM-d) δ 7.07 (q, |
| | | | | difluorophenyl) | J = 8.48 Hz, 1H), 6.93 − 7.02 |
| | | | | cyclopropyl)- | (m, 1H), 6.90 (br. s., 1H), 6.53 |
| | | | | N2,N2,6- | (s, 1H), 5.08 (br. s., 1H), 3.06 |
| | | | | trimethylthieno[2 | (s, 6H), 2.41 (s, 3H), 2.05 (br. |
| | | | | ,3-d]pyrimidine- | s., 1H), 1.27 − 1.33 (m, 2H); |
| | | | | 2,4-diamine | LCMS: ESI [M + H]⁺ = 361.1 |
| 510 | | | 8% | 2-(azetidin-1-yl)- | ¹H NMR (400 MHz, |
| | | | | 6-methyl-N-(3- | CHLOROFORM-d) δ 7.28 − |
| | | | | phenylpropyl) | 7.33 (m, 2H), 7.22 (d, J = 7.04 |
| | | | | thieno[2,3- | Hz, 3H), 6.44 (s, 1H), 4.74 |
| | | | | d]pyrimidin-4- | (br. s., 1H), 4.12 (t, J = 7.24 |
| | | | | amine | Hz, 4H), 3.59 (q, J = 6.52 Hz, |
| | | | | | 2H), 2.73 (t, J = 7.83 Hz, 2H), |
| | | | | | 2.45 (s, 3H), 2.27 − 2.37 (m, |
| | | | | | 2H), 2.00 (quin, J = 7.34 Hz, |
| | | | | | 2H); LCMS: ESI [M + H]⁺ = |
| | | | | | 339.2 |
| 511 | | | 14% | 2-(3,3- | ¹H NMR (400 MHz, |
| | | | | difluoroazetidin- | CHLOROFORM-d) δ 7.29 − |
| | | | | 1-yl)-6-methyl- | 7.35 (m, 2H), 7.19 − 7.26 (m, |
| | | | | N-(3- | 3H), 6.50 (s, 1H), 4.44 (t, J = |
| | | | | phenylpropyl) | 11.54 Hz, 4H), 3.56 − 3.64 (m, |
| | | | | thieno[2,3- | 2H), 2.75 (t, J = 7.24 Hz, 2H), |
| | | | | d]pyrimidin-4- | 2.47 (s, 3H), 1.97 − 2.07 (m, |
| | | | | amine | 2H); LCMS: ESI [M + H]⁺ = |
| | | | | | 375.1 |
| 512 | | | 9% | 2-(3- | ¹H NMR (400 MHz, |
| | | | | fluoroazetidin-1- | CHLOROFORM-d) δ 7.29 − |
| | | | | yl)-6-methyl-N- | 7.35 (m, 2H), 7.20 − 7.26 (m, |
| | | | | (3- | 3H), 6.53 (br. s., 1H), 5.46 (br. |
| | | | | phenylpropyl) | s., 1H), 5.32 (br. s., 1H), 4.47 |
| | | | | thieno[2,3- | (br. s., 2H), 4.33 (br. s., 1H), |
| | | | | d]pyrimidin-4- | 4.28 (br. s., 1H), 3.61 (q, J = |
| | | | | amine | 6.13 Hz, 2H), 2.75 (t, J = 6.65 |
| | | | | | Hz, 2H), 2.44 (s, 3H), 1.98 − |
| | | | | | 2.07 (m, J = 6.65 Hz, 2H); |
| | | | | | LCMS: ESI [M + H]⁺ = 357.1 |
| 513 | | | 24% | 2-(3,3- | ¹H NMR (400 MHz, |
| | | | | difluoropyrrolidin- | CHLOROFORM-d) δ 7.29 − |
| | | | | 1-yl)-6-methyl- | 7.34 (m, 2H), 7.20 − 7.25 (m, |
| | | | | N-(3- | 3H), 6.46 (s, 1H), 4.81 (br. s., |
| | | | | phenylpropyl) | 1H), 3.91 (t, J = 13.69 Hz, |
| | | | | thieno[2,3- | 2H), 3.78 (t, J = 7.04 Hz, 2H), |
| | | | | d]pyrimidin-4- | 3.59 (q, J = 6.13 Hz, 2H), 2.74 |
| | | | | amine | (t, J = 7.04 Hz, 2H), 2.46 (s, |
| | | | | | 3H), 2.36 − 2.45 (m, 2H), 2.01 |
| | | | | | (quin, J = 7.14 Hz, 2H); |
| | | | | | LCMS: ESI [M + H]⁺ = 389.1 |
| 514 | | | 12% | (R)-6-methyl-2- | ¹H NMR (400 MHz, |
| | | | | (2- | CHLOROFORM-d) δ 7.28 − |
| | | | | methylpyrrolidin- | 7.33 (m, 2H), 7.22 (d, J = 7.43 |
| | | | | 1-yl)-N-(3- | Hz, 3H), 6.45 (s, 1H), 4.78 |
| | | | | phenylpropyl) | (br. t, J = 5.50 Hz, 1H), 3.53 − |
| | | | | thieno[2,3- | 3.63 (m, 6H), 2.73 (t, J = 7.43 |
| | | | | d]pyrimidin-4- | Hz, 2H), 2.44 (s, 3H), 1.98 − |
| | | | | amine | 2.07 (m, 2H), 1.90 − 1.98 (m, |
| | | | | | 4H); LCMS: ESI [M + H]⁺ = |
| | | | | | 367.2 |
| 515 | | | 17% | 2-(4,4- | ¹H NMR (400 MHz, |
| | | | | difluoropiperidin- | CHLOROFORM-d) δ 7.28 − |
| | | | | 1-yl)-6-methyl- | 7.34 (m, 2H), 7.18 − 7.25 (m, |
| | | | | N-(3- | 3H), 6.48 (br. s., 1H), 3.93 (br. |
| | | | | phenylpropyl) | s., 4H), 3.58 (q, J = 6.26 Hz, |
| | | | | thieno[2,3- | 2H), 2.74 (t, J = 7.24 Hz, 2H), |
| | | | | d]pyrimidin-4- | 2.45 (s, 3H), 1.92 − 2.06 (m, |
| | | | | amine | 6H); LCMS: ESI [M + H]⁺ = |
| | | | | | 403.2 |
| 516 | | | 29% | 6-methyl-N-(3- | ¹H NMR (400 MHz, |
| | | | | phenylpropyl)-2- | CHLOROFORM-d) δ 7.27 − |
| | | | | (pyrrolidin-1- | 7.33 (m, 2H), 7.21 (d, J = 6.65 |
| | | | | yl)thieno[2,3- | Hz, 3H), 6.70 (br. s., 1H), 3.46 − |
| | | | | d]pyrimidin-4- | 3.62 (m, J = 7.43 Hz, 6H), |
| | | | | amine | 2.72 (t, J = 7.04 Hz, 2H), 2.31 |
| | | | | | (s, 3H), 1.93 − 2.07 (m, J = |
| | | | | | 5.48 Hz, 6H); LCMS: ESI |
| | | | | | [M + H]⁺ = 353.2 |
| 517 | | | 46% | 6-methyl-N-(3- | ¹H NMR (400 MHz, |
| | | | | phenylpropyl)-2- | CHLOROFORM-d) δ 7.28 − |
| | | | | (piperidin-1- | 7.34 (m, 2H), 7.22 (d, J = 7.04 |
| | | | | yl)thieno[2,3- | Hz, 3H), 6.44 (s, 1H), 4.72 |
| | | | | d]pyrimidin-4- | (br. s., 1H), 3.75 (br. s., 4H), |
| | | | | amine | 3.53 − 3.64 (m, 2H), 2.74 (t, J = |
| | | | | | 7.24 Hz, 2H), 2.44 (s, 3H), |
| | | | | | 2.00 (quin, J = 6.85 Hz, 2H), |
| | | | | | 1.61 (br. s., 6H); LCMS: ESI |
| | | | | | [M + H]⁺ = 367.2 |
| 518 | | | 22% | 2-(3- | ¹H NMR (400 MHz, |
| | | | | methoxyazetidin- | CHLOROFORM-d) δ 7.28 − |
| | | | | 1-yl)-6-methyl- | 7.34 (m, 2H), 7.19 − 7.25 (m, |
| | | | | N-(3- | J = 7.04 Hz, 3H), 6.45 (s, 1H), |
| | | | | phenylpropyl) | 4.78 (br. s., 1H), 4.23 − 4.32 |
| | | | | thieno[2,3- | (m, 3H), 3.96 − 4.01 (m, J = |
| | | | | d]pyrimidin-4- | 9.00 Hz, 2H), 3.59 (q, J = 6.52 |
| | | | | amine | Hz, 2H), 3.32 − 3.37 (m, 3H), |
| | | | | | 2.73 (t, J = 7.24 Hz, 2H), 2.45 |
| | | | | | (s, 3H), 2.00 (quin, J = 7.24 |
| | | | | | Hz, 2H); LCMS: ESI [M + |
| | | | | | H]⁺ = 369.1 |
| 519 | | | 11% | 6-methyl-N-(3- | ¹H NMR (400 MHz, |
| | | | | phenylpropyl)-2- | CHLOROFORM-d) δ 7.29 − |
| | | | | (thiazolidin-3- | 7.34 (m, 2H), 7.20 − 7.25 (m, |
| | | | | yl)thieno[2,3- | 3H), 6.46 (s, 1H), 4.78 (d, J = |
| | | | | d]pyrimidin-4- | 2.35 Hz, 2H), 3.98 (dt, J = |
| | | | | amine | 2.15, 6.36 Hz, 2H), 3.59 (q, |
| | | | | | J = 5.61 Hz, 2H), 3.04 (dt, J = |
| | | | | | 2.35, 6.06 Hz, 2H), 2.74 (t, J = |
| | | | | | 7.43 Hz, 2H), 2.46 (s, 3H), |
| | | | | | 2.01 (quin, J = 7.14 Hz, 2H); |
| | | | | | LCMS: ESI [M + H]⁺ = 371.1 |
| 520 | | | 8% | N2, N2-diethyl-6- | ¹H NMR (400 MHz, |
| | | | | methyl-N4-(3- | CHLOROFORM-d) δ 7.28 − |
| | | | | phenylpropyl)thieno | 7.33 (m, 2H), 7.19 − 7.24 (m, |
| | | | | [2,3- | J = 7.04 Hz, 3H), 6.43 (s, 1H), |
| | | | | d]pyrimidine-2,4- | 4.68 (br. s., 1H), 3.54 − 3.66 |
| | | | | diamine | (m, 6H), 2.73 (t, J = 7.63 Hz, |
| | | | | | 2H), 2.44 (s, 3H), 1.97 − 2.04 |
| | | | | | (m, J = 7.24, 7.24 Hz, 2H), |
| | | | | | 1.18 (dt, J = 1.76, 6.95 Hz, |
| | | | | | 6H); LCMS: ESI [M + H]⁺ = |
| | | | | | 355.2 |
| 521 | | | 30% | N2,6-dimethyl- | 2.35, 5.09 Hz, 3H), 2.74 (t, J = |
| | | | | N4-(3- | ¹H NMR (400 MHz, |
| | | | | phenylpropyl) | CHLOROFORM-d) δ 7.28 − |
| | | | | thieno[2,3-d] | 7.34 (m, 2H), 7.19 − 7.25 (m, |
| | | | | pyrimidine-2,4- | J = 7.04 Hz, 3H), 6.44 (s, 1H), |
| | | | | diamine | 4.73 (br. s., 2H), 3.58 (q, J = |
| | | | | | 6.13 Hz, 2H), 2.99 (dd, J = |
| | | | | | 7.43 Hz, 2H), 2.45 (s, 3H), |
| | | | | | 2.01 (quin, J = 6.75 Hz, 2H); |
| | | | | | LCMS: ESI [M + H]⁺ = 313.1 |
| 522 | | | 14% | N2-cyclopropyl- | ¹H NMR (400 MHz, |
| | | | | 6-methyl-N4-(3- | CHLOROFORM-d) δ 7.28 − |
| | | | | phenylpropyl) | 7.34 (m, 2H), 7.19 − 7.24 (m, |
| | | | | thieno[2,3- | J = 6.26 Hz, 3H), 6.45 (s, 1H), |
| | | | | d]pyrimidine-2,4- | 4.96 (br. s., 1H), 4.74 (br. s., |
| | | | | diamine | 1H), 3.58 (q, J = 6.26 Hz, 2H), |
| | | | | | 2.79 (br. s., 1H), 2.74 (t, J = |
| | | | | | 7.63 Hz, 2H), 2.45 (s, 3H), |
| | | | | | 2.01 (quin, J = 7.04 Hz, 2H), |
| | | | | | 0.72 − 0.80 (m, J = 6.65 Hz, |
| | | | | | 2H), 0.51 − 0.56 (m, J = 1.17 |
| | | | | | Hz, 2H); LCMS: ESI |
| | | | | | [M + H]⁺ = 339.1 |
| 523 | | | 54% | N-((1R,2S)-2- | ¹H NMR (400 MHz, |
| | | | | (3,4- | CHLOROFORM-d) δ 7.06 − |
| | | | | difluorophenyl) | 7.15 (m, 1H), 6.94 − 7.02 (m, |
| | | | | cyclopropyl)-2- | 1H), 6.88 − 6.94 (m, 1H), 6.57 |
| | | | | (3,3- | (s, 1H), 5.18 (br. s., 1H), 3.82 |
| | | | | difluoropyrrolidin- | (t, J = 13.50 Hz, 2H), 3.65 − |
| | | | | 1-yl)-6- | 3.75 (m, 2H), 2.98 − 3.05 (m, |
| | | | | methylthieno | 1H), 2.43 (s, 3H), 2.31 − 2.40 |
| | | | | [2,3-d]pyrimidin-4- | (m, 2H), 2.01 − 2.09 (m, 1H), |
| | | | | amine | 1.25 − 1.38 (m, 2H); LCMS: |
| | | | | | ESI [M + H]⁺ = 423.1 |
| 524 | | | 34% | 2-(3,3- | ¹H NMR (400 MHz, |
| | | | | difluoroazetidin- | CHLOROFORM-d) δ 7.00 − |
| | | | | 1-yl)-N-((1R,2S)- | 7.16 (m, 2H), 6.92 − 6.99 (m, |
| | | | | 2-(3,4- | J = 6.65 Hz, 1H), 6.61 (s, 1H), |
| | | | | difluorophenyl) | 5.25 (br. s., 1H), 4.35 (t, J = |
| | | | | cyclopropyl)-6- | 12.13 Hz, 4H), 2.97 − 3.03 (m, |
| | | | | methylthieno | J = 2.74 Hz, 1H), 2.45 (s, 3H), |
| | | | | [2,3-d]pyrimidin-4- | 2.03 − 2.10 (m, 1H), 1.25 − |
| | | | | amine | 1.34 (m, 2H); LCMS: ESI |
| | | | | | [M + H]⁺ = 409.1 |
| 525 | | | 43% | N4-((1R,2S)-2- | ¹H NMR (400 MHz, |
| | | | | (3,4- | CHLOROFORM-d) δ 7.00 − |
| | | | | difluorophenyl) | 7.14 (m, 2H), 6.90 − 6.96 (m, |
| | | | | cyclopropyl)- | J = 5.48 Hz, 1H), 6.56 (s, 1H), |
| | | | | N2,6- | 5.22 (s, 1H), 4.78 (br. s., 1H), |
| | | | | dimethylthieno[2, | 3.00 (br. s., 1H), 2.90 − 2.96 |
| | | | | 3-d]pyrimidine- | (m, J = 2.74 Hz, 3H), 2.41 (br. |
| | | | | 2,4-diamine | s., 3H), 2.06 (t, J = 6.65 Hz, |
| | | | | | 1H), 1.26 − 1.35 (m, J = 3.13 |
| | | | | | Hz, 2H); LCMS: ESI [M + |
| | | | | | H]⁺ = 347.1 |
| 526 | | | 4% | N4-((1R,2S)-2- | ¹H NMR (400 MHz, |
| | | | | (3,4- | CHLOROFORM-d) δ 7.09 − |
| | | | | difluorophenyl) | 7.19 (m, J = 8.22 Hz, 1H), |
| | | | | cyclopropyl)-6- | 6.98 − 7.05 (m, J = 2.35 Hz, |
| | | | | methyl-N2- | 1H), 6.92 (br. s., 1H), 6.57 (s, |
| | | | | (oxetan-3- | 1H), 5.17 − 5.26 (m, 2H), 5.02 − |
| | | | | yl)thieno[2,3- | 5.11 (m, J = 3.13 Hz, 1H), |
| | | | | d]pyrimidine-2,4- | 4.79 − 4.88 (m, 2H), 4.53 (td, J = |
| | | | | diamine | 3.33, 5.87 Hz, 2H), 2.97 − |
| | | | | | 3.04 (m, J = 2.35 Hz, 1H), |
| | | | | | 2.43 (s, 3H), 2.02 − 2.10 (m, J = |
| | | | | | 2.35 Hz, 1H), 1.33 (dd, J = |
| | | | | | 5.48, 8.22 Hz, 2H); LCMS: |
| | | | | | ESI [M + H]⁺ = 389.1 |
| 527 | | | 16% | N4-((1R,2S)-2- | ¹H NMR (400 MHz, |
| | | | | (3,4- | CHLOROFORM-d) δ 7.04 − |
| | | | | difluorophenyl) | 7.15 (m, J = 5.48 Hz, 2H), |
| | | | | cyclopropyl)-6- | 6.96 (s, 1H), 6.59 (s, 1H), 5.23 |
| | | | | methylthieno[2,3 | (br. s., 1H), 4.70 (br. s., 2H), |
| | | | | -d]pyrimidine- | 2.98 (br. s., 1H), 2.42 (s, 3H), |
| | | | | 2,4-diamine | 2.08 (br. s., 1H), 1.32 (br. s., |
| | | | | | 2H); LCMS: ESI [M + H]⁺ = |
| | | | | | 333.1 |
| 528 | | | 78% | 6-methyl-2- | ¹H NMR (400 MHz, |
| | | | | (pyrrolidin-1-yl)- | CHLOROFORM-d) δ 7.57 − |
| | | | | N-(3-(4′- | 7.62 (m, 2H), 7.50 (d, J = 8.19 |
| | | | | (trifluoromethoxy)- | Hz, 2H), 7.28 − 7.32 (m, 4H), |
| | | | | [1,1′- | 6.40 (s, 1H), 3.64 (q, J = 6.72 |
| | | | | biphenyl]-4- | Hz, 2H), 3.57 (t, J = 6.54 Hz, |
| | | | | yl)propyl)thieno | 4H), 2.79 (t, J = 7.40 Hz, 2H), |
| | | | | [2,3-d]pyrimidin- | 2.36 − 2.41 (m, 3H), 2.05 |
| | | | | 4-amine | (quin, J = 7.15 Hz, 2H), 1.90 − |
| | | | | | 1.96 (m, J = 6.66, 6.66 Hz, |
| | | | | | 4H); LCMS: ESI [M + H]⁺ = |
| | | | | | 513.2 |
| 529 | | | 31% | N2-(furan-2- | ¹H NMR (400 MHz, |
| | | | | ylmethyl)-6- | CHLOROFORM-d) δ 8.28 (s, |
| | | | | methyl-N4-(3- | 1H), 7.28 − 7.33 (m, 2H), 7.17 − |
| | | | | phenylpropyl) | 7.24 (m, 3H), 6.61 (s, 1H), |
| | | | | thieno[2,3- | 6.21 − 6.32 (m, 2H), 4.58 (d, |
| | | | | d]pyrimidine-2,4- | J = 4.16 Hz, 2H), 3.64 (q, J = |
| | | | | diamine | 6.44 Hz, 2H), 2.74 (t, J = 7.34 |
| | | | | | Hz, 2H), 2.42 (s, 3H), 2.04 (t, |
| | | | | | J = 7.15 Hz, 2H); LCMS: ESI |
| | | | | | [M + H]⁺ = 379.2 |
| 530 | | | 55% | N-(3-(4′-chloro- | ¹H NMR (400 MHz, |
| | | | | [1,1′-biphenyl]-4- | CHLOROFORM-d) δ 7.50 |
| | | | | yl)propyl)-6- | (dd, J = 6.54, 8.25 Hz, 4H), |
| | | | | methyl-2- | 7.40 (d, J = 8.56 Hz, 2H), 7.27 − |
| | | | | (pyrrolidin-1- | 7.31 (m, 2H), 6.61 (s, 1H), |
| | | | | yl)thieno[2,3- | 3.53 − 3.70 (m, 6H), 2.78 (t, J = |
| | | | | d]pyrimidin-4- | 7.34 Hz, 2H), 2.32 (s, 3H), |
| | | | | amine | 2.06 (quin, J = 7.15 Hz, 2H), |
| | | | | | 1.88 − 2.00 (m, 4H); LCMS: |
| | | | | | ESI [M + H]⁺ = 463.2 |
| 531 | | | 3% | N4-(3-(4′-chloro- | ¹H NMR (400 MHz, |
| | | | | [1,1′-biphenyl]-4- | METHANOL-d4) δ 7.57 (d, |
| | | | | yl)propyl)- | J = 8.44 Hz, 2H), 7.47 − 7.54 |
| | | | | N2,N2,6- | (m, J = 7.95 Hz, 2H), 7.38 − |
| | | | | trimethylthieno | 7.45 (m, J = 8.56 Hz, 2H), |
| | | | | [2,3-d]pyrimidine- | 7.29 (d, J = 7.95 Hz, 2H), 6.78 |
| | | | | 2,4-diamine | (s, 1H), 3.56 (t, J = 7.09 Hz, |
| | | | | | 2H), 3.07 (s, 6H), 2.75 (t, J = |
| | | | | | 7.52 Hz, 2H), 2.40 (s, 3H), |
| | | | | | 1.97 − 2.08 (m, 2H); LCMS: |
| | | | | | ESI [M + H]⁺ = 437.2 |
| 532 | | | 7% | N2,6-dimethyl- | ¹H NMR (400 MHz, |
| | | | | N4-(3-(4′- | METHANOL-d4) δ 7.67 (d, |
| | | | | (trifluoromethoxy)- | J = 8.44 Hz, 2H), 7.52 (d, J = |
| | | | | [1,1′- | 8.19 Hz, 2H), 7.31 (t, J = 7.40 |
| | | | | biphenyl]-4- | Hz, 4H), 6.81 (s, 1H), 3.57 (t, |
| | | | | yl)propyl)thieno | J = 6.97 Hz, 2H), 2.87 (s, 3H), |
| | | | | [2,3-d]pyrimidine- | 2.76 (t, J = 7.58 Hz, 2H), 2.41 |
| | | | | 2,4-diamine | (s, 3H), 2.03 (quin, J = 7.31 |
| | | | | | Hz, 2H); LCMS: ESI [M + |
| | | | | | H]⁺ = 473.2 |
| 533 | | | 14% | 2-(azetidin-1-yl)- | ¹H NMR (400 MHz, |
| | | | | 6-methyl-N-(3- | METHANOL-d4) δ 7.63 (d, |
| | | | | (4′- | J = 8.68 Hz, 2H), 7.51 (d, J = |
| | | | | (trifluoromethoxy)- | 8.07 Hz, 2H), 7.29 (d, J = 7.70 |
| | | | | [1,1′- | Hz, 4H), 7.11 (s, 1H), 4.08 (t, |
| | | | | biphenyl]-4- | J = 5.75 Hz, 2H), 3.63 (t, J = |
| | | | | yl)propyl)thieno | 7.03 Hz, 2H), 3.54 (t, J = 5.44 |
| | | | | [2,3-d]pyrimidin- | Hz, 2H), 2.77 (t, J = 7.52 Hz, |
| | | | | 4-amine | 2H), 2.52 (s, 3H), 2.20 − 2.32 |
| | | | | | (m, 2H), 2.06 (quin, J = 7.21 |
| | | | | | Hz, 2H); LCMS: ESI [M + |
| | | | | | H]⁺ = 499.3 |
| 534 | | | 57% | 2-(3,3- | ¹H NMR (400 MHz, |
| | | | | difluoropyrrolidin- | CHLOROFORM-d) δ 7.59 (d, |
| | | | | 1-yl)-6-methyl- | J = 8.68 Hz, 2H), 7.51 (d, J = |
| | | | | N-(3-(4′- | 8.07 Hz, 2H), 7.27 − 7.32 (m, |
| | | | | (trifluoromethoxy)- | 4H), 6.53 (br. s., 1H), 3.95 (t, |
| | | | | [1,1′- | J = 13.14 Hz, 2H), 3.86 (br. s., |
| | | | | biphenyl]-4- | 2H), 3.64 (q, J = 6.60 Hz, 2H), |
| | | | | yl)propyl)thieno | 2.79 (t, J = 7.27 Hz, 2H), 2.39 − |
| | | | | [2,3-d]pyrimidin- | 2.49 (m, 2H), 2.36 (s, 3H), |
| | | | | 4-amine | 2.07 (quin, J = 7.06 Hz, 2H); |
| | | | | | LCMS: ESI [M + H]⁺ = 549.2 |
| 535 | | | 47% | N2,N2,6- | ¹H NMR (400 MHz, |
| | | | | trimethyl-N4-(3- | METHANOL-d4) δ 7.68 (d, |
| | | | | (4′- | J = 8.68 Hz, 2H), 7.53 (d, J = |
| | | | | (trifluoromethoxy)- | 8.07 Hz, 2H), 7.27 − 7.38 (m, |
| | | | | [1,1′- | 4H), 6.79 (s, 1H), 3.58 (t, J = |
| | | | | biphenyl]-4- | 7.09 Hz, 2H), 3.09 (s, 6H), |
| | | | | yl)propyl)thieno | 2.77 (t, J = 7.46 Hz, 2H), 2.42 |
| | | | | [2,3-d]pyrimidine- | (s, 3H), 1.98 − 2.11 (m, 2H); |
| | | | | 2,4-diamine | LCMS: ESI [M + H]⁺ = 487.2 |
| 536 | | | 33% | N5,N5-dimethyl- | ¹H NMR (399 MHz, |
| | | | | N7-(3-(4′- | METHANOL-d4) δ 8.49 (s, |
| | | | | (trifluoromethox | 1H), 7.68 (d, J = 8.56 Hz, 2H), |
| | | | | y)-[1,1′- | 7.53 (d, J = 7.79 Hz, 2H), 7.32 |
| | | | | biphenyl]-4- | (t, J = 6.81 Hz, 4H), 3.61 (t, |
| | | | | yl)propyl)thiazolo | J = 7.20 Hz, 2H), 3.12 (s, 6H), |
| | | | | [5,4- | 2.78 (t, J = 7.59 Hz, 2H), 2.05 |
| | | | | d]pyrimidine-5,7- | (quin, J = 7.30 Hz, 2H); |
| | | | | diamine | LCMS: ESI [M + H]⁺ = 474.1 |
| 537 | | | 25% | 6-methyl-N4-(3- | ¹H NMR (399 MHz, |
| | | | | (4′- | METHANOL-d4) δ 7.66 (d, |
| | | | | (trifluoromethox | J = 8.56 Hz, 2H), 7.51 (d, J = |
| | | | | y)-[1,1′- | 8.18 Hz, 2H), 7.31 (t, J = 7.40 |
| | | | | biphenyl]-4- | Hz, 4H), 6.83 (s, 1H), 3.53 (t, |
| | | | | yl)propyl)thieno | J = 7.01 Hz, 2H), 2.75 (t, J = |
| | | | | [2,3-d]pyrimidine- | 7.59 Hz, 2H), 2.40 (s, 3H), |
| | | | | 2,4-diamine | 2.01 (quin, J = 7.40 Hz, 2H); |
| | | | | | LCMS: ESI [M + H]⁺ = 459.1 |
| 538 | | | 32% | N2-ethyl-N2,6- | ¹H NMR (399 MHz, |
| | | | | dimethyl-N4-(3- | METHANOL-d4) δ 7.66 (d, |
| | | | | (4′- | J = 8.56 Hz, 2H), 7.51 (d, J = |
| | | | | (trifluoromethoxy)- | 7.79 Hz, 2H), 7.29 (d, J = 7.79 |
| | | | | [1,1′- | Hz, 2H), 7.32 (d, J = 8.56 Hz, |
| | | | | biphenyl]-4- | 2H), 6.77 (s, 1H), 3.51 − 3.63 |
| | | | | yl)propyl)thieno | (m, 4H), 3.04 (s, 3H), 2.75 (t, |
| | | | | [2,3-d]pyrimidine- | J = 7.40 Hz, 2H), 2.40 (s, 3H), |
| | | | | 2,4-diamine | 2.02 (quin, J = 7.20 Hz, 2H), |
| | | | | | 1.10 (t, J = 6.81 Hz, 3H); |
| | | | | | LCMS: ESI [M + H]⁺ = 501.2 |
| 539 | | | 5% | N4-(3-(4′- | ¹H NMR (399 MHz, |
| | | | | (dimethylamino)- | METHANOL-d4) δ 7.46 (d, |
| | | | | [1,1′-biphenyl]-4- | J = 8.95 Hz, 4H), 7.22 (d, J = |
| | | | | yl)propyl)-N2,6- | 7.79 Hz, 2H), 6.80 − 6.87 (m, |
| | | | | dimethylthieno[2, | 3H), 3.57 (t, J = 7.01 Hz, 2H), |
| | | | | 3-d]pyrimidine- | 2.96 (s, 6H), 2.87 (s, 3H), 2.72 |
| | | | | 2,4-diamine | (t, J = 7.20 Hz, 2H), 2.41 (s, |
| | | | | | 3H), 2.02 (td, J = 7.20, 14.40 |
| | | | | | Hz, 2H); LCMS: ESI [M + |
| | | | | | H]⁺ = 432.2 |
| 540 | | | 6% | 6-methyl-2-(2- | ¹H NMR (399 MHz, |
| | | | | oxa-6- | METHANOL-d4) δ 7.71 (d, |
| | | | | azaspiro [3.3 ]hept | J = 8.56 Hz, 2H), 7.55 (d, J = |
| | | | | an-6-yl)-N-(3-(4′- | 7.79 Hz, 2H), 7.32 (t, J = 8.76 |
| | | | | (trifluoromethox | Hz, 4H), 6.82 (s, 1H), 4.76 (s, |
| | | | | y)-[1,1′- | 4H), 4.11 (s, 4H), 3.55 (t, J = |
| | | | | biphenyl]-4- | 7.20 Hz, 2H), 2.76 (t, J = 7.20 |
| | | | | yl)propyl)thieno[ | Hz, 2H), 2.41 (s, 3H), 2.01 |
| | | | | 2,3-d]pyrimidin- | (quin, J = 7.20 Hz, 2H); |
| | | | | 4-amine | LCMS: ESI [M + H]⁺ = 541.2 |
| 541 | | | 25% | N-(3-(4′-chloro- | ¹H NMR (399 MHz, |
| | | | | [1,1′-biphenyl]-4- | METHANOL-d4) δ 7.54 − |
| | | | | yl)propyl)-6- | 7.60 (m, 2H), 7.47 − 7.53 (m, |
| | | | | methyl-2- | J = 8.17 Hz, 2H), 7.38 − 7.44 |
| | | | | (pyrrolidin-1- | (m, 2H), 7.28 (d, J = 8.18 Hz, |
| | | | | yl)thieno[2,3- | 2H), 6.77 (s, 1H), 3.56 (t, J = |
| | | | | d]pyrimidin-4- | 7.20 Hz, 2H), 3.41 − 3.48 (m, |
| | | | | amine | 4H), 2.74 (t, J = 7.40 Hz, 2H), |
| | | | | | 2.40 (s, 3H), 1.96 − 2.07 (m, |
| | | | | | 2H), 1.84 − 1.93 (m, 4H); |
| | | | | | LCMS: ESI [M + H]⁺ = 463.1 |

Scheme 5 (Route C): To a threaded pressure vial was added the appropriate starting material (0.025 mmol), followed by the desired alkoxy reagent (1 mL) and the reaction was sealed and stirred for the specified time and temperature (conditions in Table 7). Upon completion, the reaction was cooled to rt and neutralized with [TN] HCl(aq) for basic reactions or [TN] NaOH(aq) for acidic reactions. The reaction was concentrated in vacuo, and purified by prep HPLC or silica gel column chromatography, or a combination of the two, to give the pure product (Table 7).

TABLE 7
Examples from Scheme 5 (Route C)

| Cmpd. | | Starting | | | |
|---|---|---|---|---|---|
| No. | Structure | Material | Conditions | Yield | IUPAC |
| 542 | 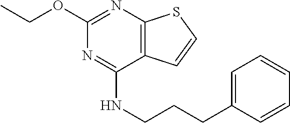 [SMILES: CCOc1nc(NCCCc2ccccc2)c2ccsc2n1] | 218 | [3.3M] NaOEt 18 h (60° C.) | 53% | 2-ethoxy-N-(3- phenylpropyl)thieno[2,3- d]pyrimidin-4-amine |
| 543 | 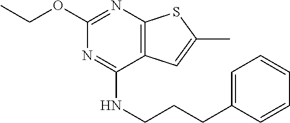 [SMILES: CCOc1nc(NCCCc2ccccc2)c2cc(C)sc2n1] | 224 | [3.3M] NaOEt 3 h (60° C.) | 74% | 2-ethoxy-6-methyl-N-(3- phenylpropyl)thieno[2,3- d]pyrimidin-4-amine |
| 545 | 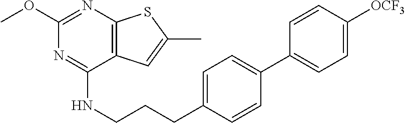 [SMILES: COc1nc(NCCCc2ccc(-c3ccc(OC(F)(F)F)cc3)cc2)c2cc(C)sc2n1] | 433 | [1M] NaOMe 6 h (85° C.) | 73% | 2-methoxy-6-methyl-N-(3- (4′-(trifluoromethoxy)-[1,1′- biphenyl]-4- yl)propyl)thieno[2,3- d]pyrimidin-4-amine |
| 546 | 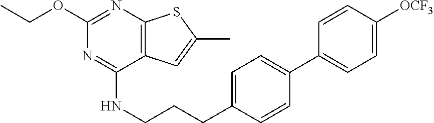 [SMILES: CCOc1nc(NCCCc2ccc(-c3ccc(OC(F)(F)F)cc3)cc2)c2cc(C)sc2n1] | 433 | [3.3M] NaOEt 1.5 h (60° C.) | 57% | 2-ethoxy-6-methyl-N-(3-(4′- (trifluoromethoxy)-[1,1′- biphenyl]-4- yl)propyl)thieno[2,3- d]pyrimidin-4-amine |
| 547 | 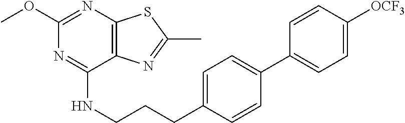 [SMILES: COc1nc(NCCCc2ccc(-c3ccc(OC(F)(F)F)cc3)cc2)c2nc(C)sc2n1] | 463 | [1M] NaOMe 3 d (60° C.) | 48% | 5-methoxy-2-methyl-N-(3- [4′-(trifluoromethoxy)-[1,1′- biphenyl]-4-yl]propyl)- [1,3]thiazolo[5,4- d]pyrimidin-7-amine |

Examples from Scheme 5 (Route C)

| No. | |
|---|---|
| Cmpd. | |
| No. | Compound Data |
| 542 | ¹H NMR (400 MHz, CHLOROFORM-d) |
| | δ 7.25 − 7.30 (m, 2H), 7.16 − 7.23 (m, 3H), |
| | 6.96 (d, J = 6.26 Hz, 1H), 6.83 (d, J = |
| | 5.87 Hz, 1H), 5.06 (br. s., 1H), 4.39 (q, J = |
| | 7.04 Hz, 2H), 3.65 (q, J = 6.65 Hz, 2H), |
| | 2.74 (t, J = 7.63 Hz, 2H), 2.01 (quin, J = |
| | 7.24 Hz, 2H), 1.40 (t, J = 7.24 Hz, 3H); |
| | LCMS: ESI [M + H]⁺ = 314.1 |
| 543 | ¹H NMR (400 MHz, CHLOROFORM-d) |
| | δ 7.28 − 7.34 (m, 2H), 7.19 − 7.25 (m, 3H), |
| | 6.51 (s, 1H), 4.39 (q, J = 7.17 Hz, 2H), |
| | 3.61 − 3.70 (m, 2H), 2.75 (t, J = 7.43 Hz, |
| | 2H), 2.48 (s, 3H), 2.02 (quin, J = 7.34 Hz, |
| | 2H), 1.42 (t, J = 7.04 Hz, 3H); LCMS: |
| | ESI [M + H]⁺ = 328.0 |
| 545 | ¹H NMR (400 MHz, CHLOROFORM-d) |
| | δ 7.58 (dd, J = 2.93, 8.41 Hz, 2H), 7.45 − |
| | 7.53 (m, 2H), 7.27 − 7.38 (m, 4H), 6.49 |
| | (br. s., 1H), 3.99 (s, 3H), 3.68 (t, J = 3.90 |
| | Hz, 2H), 2.82 (t, J = 1.00 Hz, 2H), 2.42 (s, |
| | 3H), 2.07 (br. t, J = 1.00 Hz, 2H); LCMS: |
| | ESI [M + H]⁺ = 474.2 |
| 546 | ¹H NMR (400 MHz, CHLOROFORM-d) |
| | δ 7.59 (d, J = 9.00 Hz, 2H), 7.50 (d, J = |
| | 8.22 Hz, 2H), 7.29 (dd, J = 5.28, 8.02 Hz, |
| | 4H), 6.47 (s, 1H), 4.40 (q, J = 7.04 Hz, |
| | 2H), 3.63 − 3.75 (m, 2H), 2.81 (t, J = 7.24 |
| | Hz, 2H), 2.39 (s, 3H), 1.97 − 2.16 (m, 2H), |
| | 1.42 (t, J = 7.04 Hz, 3H); LCMS: ESI |
| | [M + H]⁺ = 488.2 |
| 547 | ¹H NMR (400 MHz, METHANOL-d4) δ |
| | 7.62 − 7.70 (m, 2H), 7.51 (d, J = 8.19 Hz, |
| | 2H), 7.27 − 7.36 (m, 4H), 3.88 (s, 3H), |
| | 3.61 (t, J = 6.97 Hz, 2H), 2.78 (t, J = 7.58 |
| | Hz, 2H), 2.69 (s, 3H), 1.99 − 2.11 (m, 2H); |
| | LCMS: ESI [M + H]⁺ = 475.1 |

### Example 8: Scheme 6 Amination+Suzuki Reaction

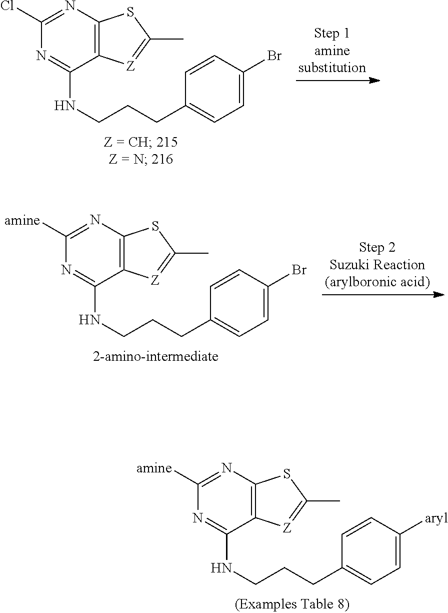

General Procedure for Scheme 6: Reaction conditions and compound data for specific examples are listed in Table 8. The starting materials, examples 215 and 216, were synthesized as detailed in Scheme 1.

Step 1: Example 215 or 216 was reacted with the appropriate amine reagent as previously described in Scheme 5, Route B, using the conditions specified (Table 8), to give the desired 2-amino-intermediate for use in Step 2.

Step 2: Following the General Suzuki Protocol, the intermediate from step 1 (0.030 mmol) and the desired aryl boronic acid (0.060 mmol) were reacted for the time and temperature specified, and the reaction was purified by prep HPLC or silica gel column chromatography, or a combination of the two, to give the desired product (Table 8).

TABLE 8
Examples from Scheme 6

| Cmpd. | | Starting | Step 1 | Step 1 | Step 2 |
|---|---|---|---|---|---|
| No. | Structure | Material | Conditions | Yield | Conditions |
| 549 | 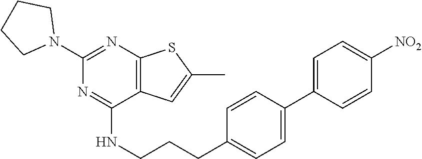 [SMILES: Cc1cc2c(NCCCc3ccc(-c4ccc([N+](=O)[O-])cc4)cc3)nc(N3CCCC3)nc2s1] | 215 | 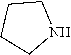 dioxane 150° C. microwave | 99% | Cs₂CO₃ 0.5 h (90° C.) |
| 550 | 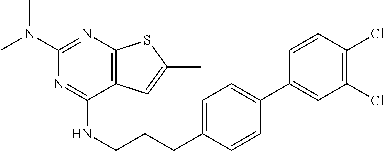 [SMILES: Cc1cc2c(NCCCc3ccc(-c4ccc(Cl)c(Cl)c4)cc3)nc(N(C)C)nc2s1] | 215 | [2M] NHMe₂ (25 eqv) 1 d (75° C.) | 99% | Cs₂CO₃ 2 h (80° C.) |
| 551 | 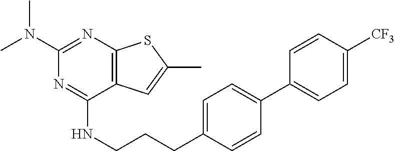 [SMILES: Cc1cc2c(NCCCc3ccc(-c4ccc(C(F)(F)F)cc4)cc3)nc(N(C)C)nc2s1] | 215 | [2M] NHMe₂ (25 eqv) 1 d (75° C.) | 99% | Cs₂CO₃ 2 h (80° C.) |
| 552 | 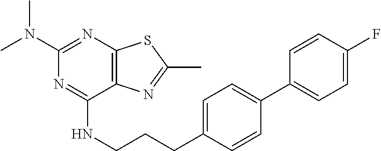 [SMILES: Cc1nc2c(NCCCc3ccc(-c4ccc(F)cc4)cc3)nc(N(C)C)nc2s1] | 215 | [2M] NHMe₂ (25 eqv) 1 d (60° C.) | 99% | Cs₂CO₃ 2 h (90° C.) |
| 553 | 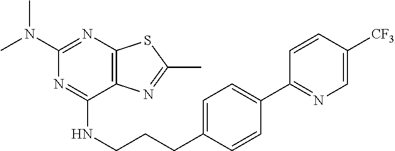 [SMILES: Cc1nc2c(NCCCc3ccc(-c4ccc(C(F)(F)F)cn4)cc3)nc(N(C)C)nc2s1] | 215 | [2M] NHMe₂ (25 eqv) 1 d (60° C.) | 99% | K₂CO₃ 2 h (90° C.) 2 h (100° C.) |
| 554 | 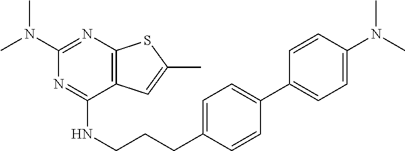 | 215 | [2M] NHMe₂ (25 eqv) 1 d (75° C.) | 99% | Cs₂CO₃ 5 h (90° C.) |
| 555 | 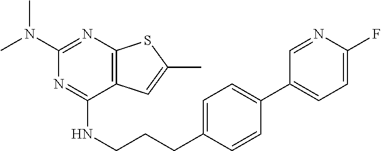 [SMILES: Cc1cc2c(NCCCc3ccc(-c4ccc(F)nc4)cc3)nc(N(C)C)nc2s1] | 215 | [2M] NHMe₂ (25 eqv) 1 d (75° C.) | 99% | Cs₂CO₃ 1.5h (90° C.) |
| 556 | 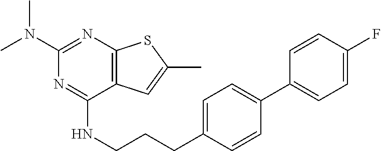 [SMILES: Cc1cc2c(NCCCc3ccc(-c4ccc(F)cc4)cc3)nc(N(C)C)nc2s1] | 215 | [2M] NHMe₂ (25 eqv) 1 d (75° C.) | 99% | Cs₂CO₃ 3h (90° C.) |

Examples from Scheme 6

| Cmpd. | Step 2 | | |
|---|---|---|---|
| No. | Yield | IUPAC | Compound Data |
| 549 | 18% | 6-methyl-N-(3-(4′- | ¹H NMR (399 MHz, DMSO-d₆) δ |
| | | nitro-[1,1′- | 8.26 − 8.33 (m, J = 8.56 Hz, 2H), |
| | | biphenyl]-4- | 7.92 − 7.99 (m, J = 8.95 Hz, 2H), |
| | | yl)propyl)-2- | 7.72 (d, J = 7.79 Hz, 2H), 7.38 (d, |
| | | (pyrrolidin-1- | J = 8.17 Hz, 2H), 6.99 (s, 1H), |
| | | yl)thieno[2,3- | 3.46 (q, J = 6.49 Hz, 2H), 3.34 − |
| | | d]pyrimidin-4- | 3.41 (m, 4H), 2.71 (t, J = 7.40 Hz, |
| | | amine | 2H), 2.38 (s, 3H), 1.93 (quin, J = |
| | | | 7.10 Hz, 2H), 1.81 (br. s., 4H); |
| | | | LCMS: ESI [M + H]⁺ = 474.2 |
| 550 | 58% | N4-(3-(31,4′- | ¹H NMR (399 MHz, |
| | | dichloro-[1,1′- | CHLOROFORM-d) δ 7.66 (d, J = |
| | | biphenyl]-4- | 1.95 Hz, 1H), 7.44 − 7.53 (m, 3H), |
| | | yl)propyl)- | 7.40 (dd, J = 1.95, 8.17 Hz, 1H), |
| | | N2,N2,6- | 7.29 (d, J = 7.79 Hz, 2H), 6.42 (s, |
| | | trimethylthieno[2, | 1H), 4.84 (br. s., 1H), 3.63 (q, J = |
| | | 3-d]pyrimidine- | 6.36 Hz, 2H), 3.16 (s, 6H), 2.78 (t, |
| | | 2,4-diamine | J = 7.59 Hz, 2H), 2.39 (s, 3H), |
| | | | 2.05 (quin, J = 7.10 Hz, 2H); |
| | | | LCMS: ESI [M + H]⁺ = 471.1 |
| 551 | 16% | N2,N2,6- | ¹H NMR (399 MHz, |
| | | trimethyl-N4-(3- | CHLOROFORM-d) δ 7.64 − 7.71 |
| | | [4′- | (m, 4H), 7.53 (d, J = 7.79 Hz, 2H), |
| | | (trifluoromethyl)- | 7.32 (d, J = 8.17 Hz, 2H), 6.55 (br. |
| | | [1,1′-biphenyl]-4- | s., 1H), 3.65 (q, J = 6.10 Hz, 2H), |
| | | yl]propyl)thieno[2, | 3.20 (s, 6H), 2.80 (t, J = 7.59 Hz, |
| | | 3-d]pyrimidine- | 2H), 2.36 (s, 3H), 2.03 − 2.16 (m, |
| | | 2,4-diamine | 2H); LCMS: ESI [M + H]⁺ = 471.2 |
| 552 | 41% | N7-(3-(4′-fluoro- | ¹H NMR (399 MHz, |
| | | [1,1′-biphenyl]-4- | CHLOROFORM-d) o 7.50 (t, J = |
| | | yl)propyl)- | 6.23 Hz, 2H), 7.44 (d, J = 7.01 Hz, |
| | | N2,N5,5- | 2H), 7.25 (d, J = 6.62 Hz, 2H), |
| | | trimethyl- | 7.01 − 7.13 (m, 2H), 5.75 (br. s., |
| | | [1,3]thiazolo[5,4- | 1H), 3.60 (q, J = 5.84 Hz, 2H), |
| | | d]pyrimidine-5,7- | 3.13 (d, J = 1.56 Hz, 6H), 2.75 (t, |
| | | diamine | J = 7.40 Hz, 2H), 2.62 (d, J = 1.17 |
| | | | Hz, 3H), 2.01 (t, J = 7.01 Hz, 2H); |
| | | | LCMS: ESI [M + H]⁺ = 422.2 |
| 553 | 15% | N2,N5,5- | ¹H NMR (399 MHz, |
| | | trimethyl-N7-(3- | CHLOROFORM-d) δ 8.94 (s, |
| | | (4-[5- | 1H), 7.97 (d, J = 7.01 Hz, 3H), |
| | | (trifluoromethyl)p | 7.83 (d, J = 8.56 Hz, 1H), 7.35 (d, |
| | | yridin-2- | J = 7.79 Hz, 2H), 5.76 (br. s., 1H), |
| | | yl]phenyl)propyl)- | 3.63 (q, J = 6.23 Hz, 2H), 3.16 (s, |
| | | [1,3]thiazolo[5,4- | 5H), 2.81 (t, J = 7.40 Hz, 2H), |
| | | d]pyrimidine-5,7- | 2.65 (s, 3H), 2.06 (quin, J = 7.20 |
| | | diamine | Hz, 2H), 1.65 (br. s., 1H); LCMS: |
| | | | ESI [M + H]⁺ = 473.2 |
| 554 | 25% | N4-(3-[4′- | ¹H NMR (399 MHz, |
| | | (dimethylamino)- | ACETONITRILE-d3) 8 7.48 (d, |
| | | [1,1′-biphenyl]-4- | J = 8.17 Hz, 4H), 7.23 (d, J = 7.79 |
| | | yl]propyl)- | Hz, 2H), 6.80 (d, J = 8.56 Hz, 2H), |
| | | N2,N2,6- | 6.69 (s, 1H), 5.83 (br. s., 1H), 3.52 |
| | | trimethylthieno[2, | (q, J = 6.49 Hz, 2H), 3.05 (s, 6H), |
| | | 3-d]pyrimidine- | 2.94 (s, 6H), 2.70 (t, J = 7.59 Hz, |
| | | 2,4-diamine | 2H), 2.39 (s, 3H), 1.97 (br. s., 2H); |
| | | | LCMS: ESI [M + H]⁺ = 446.2 |
| 555 | 43% | N4-(3-[4-(6- | ¹H NMR (399 MHz, |
| | | fluoropyridin-3- | METHANOL-d4) δ 8.34 (s, 1H), |
| | | yl)phenyl]propyl)- | 8.02 − 8.11 (m, 1H), 7.45 (d, J = |
| | | N2,N2,6- | 7.79 Hz, 2H), 7.26 (d, J = 7.79 Hz, |
| | | trimethylthieno[2, | 2H), 7.08 (dd, J = 2.14, 8.37 Hz, |
| | | 3-d]pyrimidine- | 1H), 6.74 (s, 1H), 3.51 (t, J = 7.01 |
| | | 2,4-diamine | Hz, 2H), 3.04 (s, 6H), 2.71 (t, J = |
| | | | 7.40 Hz, 2H), 2.37 (s, 3H), 1.92 − |
| | | | 2.03 (m, 2H); LCMS: ESI |
| | | | [M + H]+ = 422.2 |
| 556 | 68% | N4-(3-(4′-fluoro- | ¹H NMR (399 MHz, |
| | | [1,1′-biphenyl]-4- | METHANOL-d4) δ 7.56 (dd, J = |
| | | yl)propyl)- | 5.45, 8.56 Hz, 2H), 7.46 (d, J = |
| | | N2,N2,6- | 8.17 Hz, 2H), 7.25 (d, J = 7.79 Hz, |
| | | trimethylthieno[2, | 2H), 7.12 (t, J = 8.76 Hz, 2H), |
| | | 3-d]pyrimidine- | 6.76 (s, 1H), 3.54 (t, J = 7.20 Hz, |
| | | 2,4-diamine | 2H), 3.05 (s, 6H), 2.72 (t, J = 7.40 |
| | | | Hz, 2H), 2.38 (s, 3H), 2.00 (quin, |
| | | | J = 7.30 Hz, 2H); LCMS: ESI |
| | | | [M + H]⁺ = 421.2 |

### Example 9: Scheme 7 S_{N}Ar+Boronate Ester Conversion+Suzuki Reaction

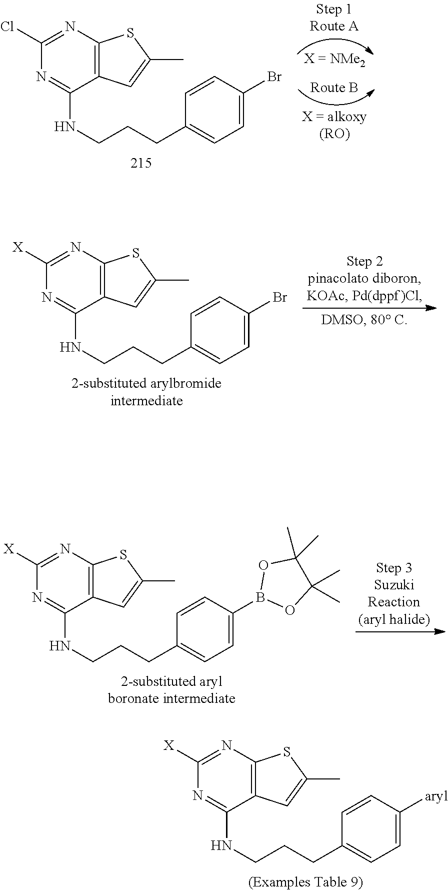

General Procedure for Scheme 7: Reaction conditions and compound data for specific examples are listed in Table 9. The starting material, example 215 was synthesized as detailed in Scheme 1.

Step 1 (Route A): To a threaded vial was added example 215 (0.19 mmol) followed by [2M] NMe₂ in isopropanol (2.5 mL). The reaction was sealed and heated to 60° C. for 16 hours. Upon completion, solvents were evaporated, then co-evaporated with MeOH (2×) and dried under vacuum, to give the intermediate 2-dimethylamino-substituted aryl bromide for use in step 2.

Step 1 (Route B): Example 215 (0.19 mmol) was dissolved in either [1N] NaOMe or [3.3 M] NaOEt (5 mL) and reacted at 75° C. for 7 hours. Upon completion, the reaction was cooled to room temperature and neutralized with [1N] HCl(aq) and extracted with DCM (3×). Organic fractions were combined, concentrated in vacuo, and purified by silica gel column chromatography to give the intermediate 2-alkoxy-substituted aryl bromide for use in step 2.

Step 2: Following the procedure in Scheme 3, step 2, the intermediate from step 1 (Routes A or B) was reacted with bis(pinacolato)diboron to give the desired 2-substituted aryl boronate intermediate (yields for steps 1-2 in Table 9).

Step 3: Following the General Suzuki Protocol, the 2-substituted aryl boronate intermediate from step 2 was reacted with the appropriate aryl halide using the conditions specified in Table 9, to give the desired product (Table 9).

TABLE 9
Examples from Scheme 7

| | | Steps 1-2 | |
|---|---|---|---|
| Cmpd. | | Route | Step 3 |
| No. | Structure | Yield | Conditions |
| 557 | 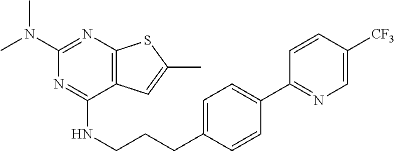 [SMILES: Cc1cc2c(NCCCc3ccc(-c4ccc(C(F)(F)F)cn4)cc3)nc(N(C)C)nc2s1] | Route A 72% | Cs₂CO₃ 5 h (90° C.) |
| 558 | 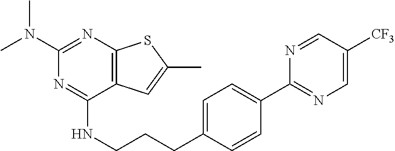 [SMILES: Cc1cc2c(NCCCc3ccc(-c4ncc(C(F)(F)F)cn4)cc3)nc(N(C)C)nc2s1] | Route A 72% | Cs₂CO₃ 3 h (90° C.) 16 h (100° C.) |
| 559 | 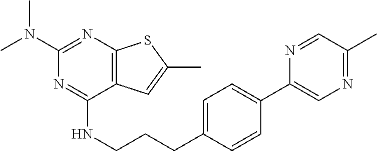 | Route A 72% | Cs₂CO₃ 2.5 h (100° C.) |
| 560 | 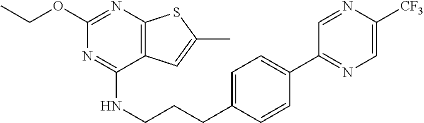 [SMILES: CCOc1nc(NCCCc2ccc(-c3cnc(C(F)(F)F)cn3)cc2)c2cc(C)sc2n1] | Route B 53% | Cs₂CO₃ 1.5 h (90° C.) |
| 561 | 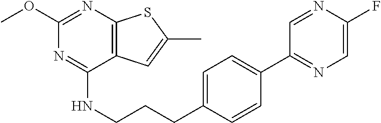 [SMILES: COc1nc(NCCCc2ccc(-c3cnc(F)cn3)cc2)c2cc(C)sc2n1] | Route B 26% | Cs₂CO₃ 1.5 h (90° C.) |

Examples from Scheme 7

| Cmpd. | Step 3 | | |
|---|---|---|---|
| No. | Yield | IUPAC | Compound Data |
| 557 | 42% | N2,N2,6-trimethyl- | ¹H NMR (399 MHz, ACETONITRILE-d3) |
| | | N4-(3-(4-[5- | δ 8.94 (s, 1H), 8.09 (d, J = 8.56 Hz, 1H), |
| | | (trifluoromethyl) | 7.93 − 8.04 (m, 3H), 7.36 (d, J = 7.79 Hz, |
| | | pyridin-2- | 2H), 6.69 (s, 1H), 5.87 (br. s., 1H), 3.53 (q, |
| | | yl]phenyl)propyl)thieno | J = 6.75 Hz, 2H), 3.05 (s, 6H), 2.76 (t, J = |
| | | [2,3-d]pyrimidine- | 7.59 Hz, 2H), 2.38 (s, 3H), 1.97 − 2.04 (m, |
| | | 2,4-diamine | 2 H); LCMS: ESI [M + H]⁺ = 472.1 |
| 558 | 27% | N2,N2,6-trimethyl- | ¹H NMR (399 MHz, ACETONITRILE-d3) |
| | | N4-(3-(4-[5- | δ 9.09 (s, 2H), 8.38 (d, J = 8.17 Hz, 2H), |
| | | (trifluoromethyl) | 7.39 (d, J = 8.17 Hz, 2H), 6.68 (d, J = 1.17 |
| | | pyrimidin-2- | Hz, 1H), 5.85 (br. s., 1H), 3.53 (q, J = 6.88 |
| | | yl]phenyl)propyl) | Hz, 2H), 3.04 (s, 6H), 2.78 (t, J = 7.59 Hz, |
| | | thieno[2,3-d]pyrimidine- | 2H), 2.38 (d, J = 1.17 Hz, 3H), 2.00 (d, J = |
| | | 2,4-diamine | 7.40 Hz, 2H); LCMS: ESI [M + H]⁺ = 473.1 |
| 559 | 37% | N2,N2,6-trimethyl- | ¹H NMR (399 MHz, ACETONITRILE-d3) |
| | | N4-(3-[4-(5- | 8 8.91 (s, 1H), 8.47 (s, 1H), 7.93 (d, J = |
| | | methylpyrazin-2- | 8.18 Hz, 2H), 7.34 (d, J = 8.17 Hz, 2H), |
| | | yl)phenyl]propyl)thieno | 6.68 (d, J = 1.17 Hz, 1H), 5.85 (br. s., 1H), |
| | | [2,3-d]pyrimidine- | 3.52 (q, J = 6.62 Hz, 2H), 3.04 (s, 6H), |
| | | 2,4-diamine | 2.74 (t, J = 7.40 Hz, 2H), 2.51 (s, 3H), 2.37 |
| | | | (d, J = 1.17 Hz, 3H), 1.98 (d, J = 7.40 Hz, |
| | | | 2H); LCMS: ESI [M + H]⁺ = 419.2 |
| 560 | 15% | 2-ethoxy-6-methyl- | ¹H NMR (399 MHz, METHANOL-d4) δ |
| | | N-(3-(4-[5- | 9.22 (s, 1H), 9.00 (s, 1H), 8.07 (d, J = 8.17 |
| | | (trifluoromethyl) | Hz, 2H), 7.39 (d, J = 8.17 Hz, 2H), 6.88 (s, |
| | | pyrazin-2- | 1H), 4.28 (q, J = 7.01 Hz, 2H), 3.57 (t, J = |
| | | yl]phenyl)propyl)thieno | 7.01 Hz, 2H), 2.80 (t, J = 7.40 Hz, 2H), |
| | | [2,3-d]pyrimidin- | 2.42 (s, 3H), 2.04 (quin, J = 7.20 Hz, 2H), |
| | | 4-amine | 1.31 (t, J = 7.01 Hz, 3H); LCMS: ESI |
| | | | [M + H]⁺ = 474.0 |
| 561 | 31% | N-(3-[4-(5- | ¹H NMR (399 MHz, METHANOL-d4) δ |
| | | fluoropyrazin-2- | 8.66 (s, 1H), 8.51 (d, J = 7.79 Hz, 1H), |
| | | yl)phenyl]propyl)-2- | 7.90 (d, J = 7.79 Hz, 2H), 7.34 (d, J = 7.79 |
| | | methoxy-6- | Hz, 2H), 6.90 (s, 1H), 3.85 (s, 3H), 3.57 (t, |
| | | methylthieno[2,3- | J = 7.01 Hz, 2H), 2.77 (t, J = 7.40 Hz, 2H), |
| | | d]pyrimidin-4-amine | 2.43 (s, 3H), 2.03 (t, J = 7.20 Hz, 2H); |
| | | | LCMS: ESI [M + H]⁺ = 410.1 |

### Example 10: Scheme 8 SNAr+Click Reaction

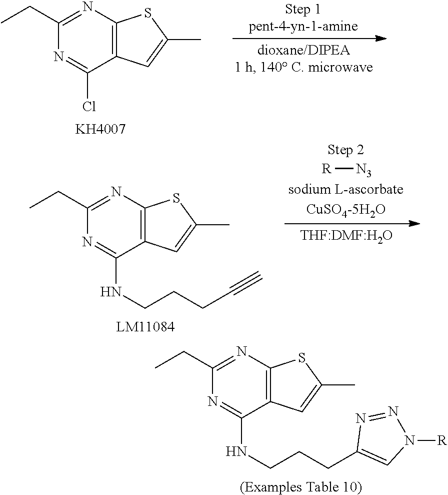

General Procedure for Scheme 8: Reaction conditions and compound data for specific examples are listed in Table 10. The starting material, KH14007, was synthesized as detailed in the building blocks section.

Step 1: Building block KH14007 (0.056) was dissolved in dioxane (1.0 mL) and DIPEA (4 eqv). Pent-4-yn-1-amine was added (2 eqv), and the solution was reacted in a microwave at 140° C. for 1 hour. Upon completion, the reaction was concentrated in vacuo, and purified by silica gel column chromatography to give intermediate LM11084: 80% yield; IUPAC: 2-ethyl-6-methyl-N-(pent-4-yn-1-yl)thieno[2,3-d]pyrimidin-4-amine, LCMS: ESI [M+H]⁺=260.0.

Step 2: Following the General Click Protocol, intermediate LM11084 was reacted with the azide derivative (RN₃) and using the conditions specified in Table 10. Upon completion, the reaction was then cooled to room temperature, concentrated in vacuo. Saturated NaS₂O₃(aq) (5 mL) was added and the reaction mixture was extracted with DCM (3×). The organic fractions were combined, concentrated, and the reaction was purified by prep HPLC to give the desired product (Table 10).

TABLE 10
Examples from Scheme 8

| Cmpd. | | | | | |
|---|---|---|---|---|---|
| No. | Structure | RN₃ | Conditions | Yield | IUPAC |
| 562 | 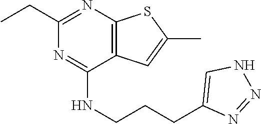 [SMILES: CCc1nc(NCCCc2c[nH]nn2)c2cc(C)sc2n1] | NaN₃ | 3 h (85° C.), 16 h (50° C.) | 8% | N-(3-(1H-1,2,3- triazol-4-yl)propyl)-2- ethyl-6- methylthieno[2,3- d]pyrimidin-4-amine |
| 563 | 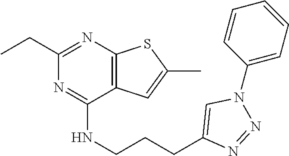 [SMILES: CCc1nc(NCCCc2cn(-c3ccccc3)nn2)c2cc(C)sc2n1] | 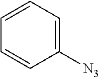 | 4 h (40° C.) | 61% | 2-ethyl-6-methyl-N- (3-(1-phenyl-1H- 1,2,3-triazol-4- yl)propyl)thieno[2,3- d]pyrimidin-4-amine |

Examples from Scheme 8

| Cmpd. | Compound Data |
|---|---|
| No. | ¹H NMR (400 MHz, CHLOROFORM- |
| 562 | d) δ 7.54 (d, J = 1.4 Hz, 1H), 6.76 (q, J = |
| | 1.3 Hz, 1H), 5.42 (s, 1H), 3.74 (q, J = |
| | 6.5 Hz, 2H), 3.00 (qd, J = 7.7, 1.4 Hz, |
| | 2H), 2.91 − 2.83 (m, 2H), 2.55 (q, J = |
| | 1.2 Hz, 3H), 2.03 (q, J = 6.3 Hz, 2H), |
| | 1.39 (tt, J = 7.6, 1.2 Hz, 3H); LCMS: |
| | ESI [M + H]⁺ = 303.2 |
| | ¹H NMR (400 MHz, CHLOROFORM- |
| | d) δ 7.74 (d, J = 2.1 Hz, 1H), 7.66 (dd, |
| | J = 8.0, 2.2 Hz, 2H), 7.51 (td, J = 8.3, |
| | 7.8, 2.1 Hz, 2H), 7.46 − 7.37 (m, 1H), |
| | 6.73 (t, J = 1.9 Hz, 1H), 5.58 (s, 1H), |
| | 3.75 (qd, J = 6.4, 2.1 Hz, 2H), 2.94 (td, |
| | J = 7.0, 2.1 Hz, 2H), 2.82 (qd, J = 7.6, |
| | 2.1 Hz, 2H), 2.50 − 2.45 (m, 3H), 2.13 |
| | (qd, J = 6.8, 2.0 Hz, 2H), 1.33 (td, J = |
| | 7.6, 2.1 Hz, 3H); LCMS: ESI |
| | [M + H]⁺ = 379.2 |

### Example 11: Scheme 9 Amidation

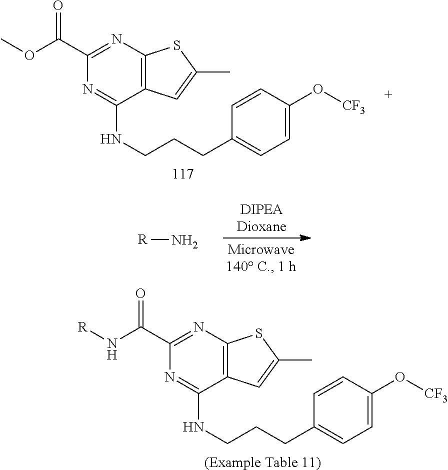

General Procedure for Scheme 9: Reaction conditions and compound data for specific examples are listed in Table 11. The starting material, example 117, was synthesized as detailed in the building blocks section.

Example 117 (13.4 mg, 0.032 mmol) was added to a microwave vial followed by the desired commercially available RNH₂ reagent (0.128 mmol) and the vial was sealed and reacted in a microwave for the specified time and temperature. Upon completion, the reaction was cooled to room temperature and concentrated in vacuo. The reaction was then purified by prep HPLC to give the desired product (Table 11).

TABLE 11
Examples from Scheme 9

| Cmpd. | | RNH₂ | | |
|---|---|---|---|---|
| No. | Structure | Conditions | Yield | IUPAC |
| 564 | 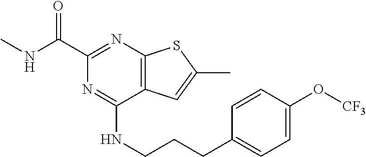 [SMILES: CNC(=O)c1nc(NCCCc2ccc(OC(F)(F)F)cc2)c2cc(C)sc2n1] | 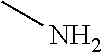 1 h (190° C.) | 52% | N,6-dimethyl-4-((3-(4- (trifluoromethoxy)phenyl) propyl)amino)thieno[2,3- d]pyrimidine-2-carboxamide |
| 565 | 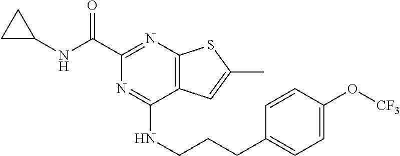 [SMILES: Cc1cc2c(NCCCc3ccc(OC(F)(F)F)cc3)nc(C(=O)NC3CC3)nc2s1] | 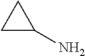 1 h (200° C.) | 17% | N-cyclopropyl-6-methyl-4-((3- (4- (trifluoromethoxy)phenyl) propyl)amino)thieno[2,3- d]pyrimidine-2-carboxamide |

Examples from Scheme 9

| Cmpd. | |
|---|---|
| No. | Compound Data |
| 564 | ¹H NMR (400 MHz, CHLOROFORM-d) δ 7.88 |
| | (br. s., 1H), 7.20 − 7.25 (m, J = 8.61 Hz, 2H), 7.13 − |
| | 7.18 (m, 2H), 6.73 (s, 1H), 5.15 (br. s., 1H), |
| | 3.70 − 3.74 (m, 2H), 3.05 (d, J = 5.09 Hz, 3H), |
| | 2.78 (t, J = 7.63 Hz, 2H), 2.58 (s, 3H), 2.05 (quin, |
| | J = 7.14 Hz, 2H); LCMS: ESI [M + H]⁺ = 425.2 |
| 565 | ¹H NMR (400 MHz, CHLOROFORM-d) δ 7.84 |
| | (br. s., 1H), 7.24 (d, J = 7.83 Hz, 2H), 7.14 (d, J = |
| | 7.83 Hz, 3H), 3.71 (d, J = 5.48 Hz, 2H), 2.94 (br. |
| | s., 1H), 2.80 (t, J = 7.04 Hz, 2H), 2.57 (s, 3H), |
| | 2.04 − 2.19 (m, 2H), 0.90 (d, J = 7.04 Hz, 2H), |
| | 0.67 (br. s., 2H); LCMS: ESI [M + H]⁺ = 451.2 |

### Example 12: Scheme 10 Suzuki Reaction+Boc Deprotection

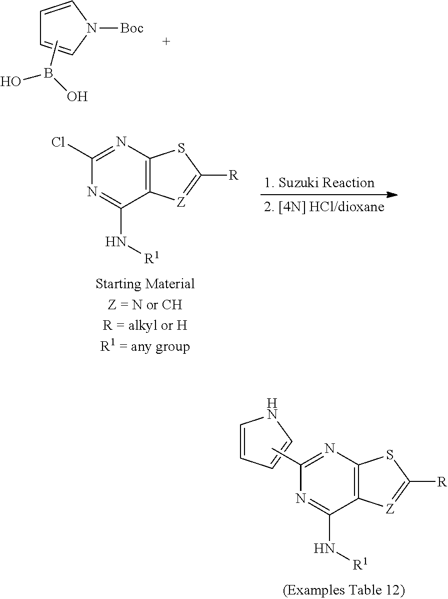

General Procedure for Scheme 10: Reaction conditions and compound data for specific examples are listed in Table 12. The Starting materials are examples that were synthesized as described in either Scheme 1 or Scheme 4.

Step 1: Following the General Suzuki Protocol, the starting material (0.060 mmol) and the desired boc-protected pyrrole boronic acid (0.120 mmol) were reacted for the specified time and temperature (conditions in Table 12). The boc-protected product was purified by silica gel column chromatography, then dissolved in [4N] HCl/dioxane (4 mL) and stirred overnight at room temperature. Upon completion, the reaction was concentrated in vacuo and purified by prep HPLC to give the desired product (Table 12).

TABLE 12
Examples from Scheme 10

| Cmpd. | | Starting | Step 1 | Steps 1-2 | |
|---|---|---|---|---|---|
| No. | Structure | Material | Conditions | Yield | IUPAC |
| 566 | 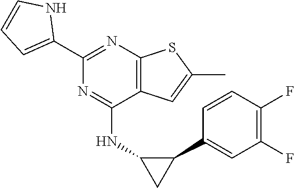 [SMILES: Cc1cc2c(N[C@H]3C[C@@H]3c3ccc(F)c(F)c3)nc(-c3ccc[nH]3)nc2s1] | 222 | Cs₂CO₃ 2 h (80° C.) | 52% | N-((1S,2R)-2-(3,4- difluorophenyl) cyclopropyl)- 6-methyl- 2-(1H-pyrrol-2- yl)thieno[2,3- d]pyrimidin-4- amine |
| 567 | 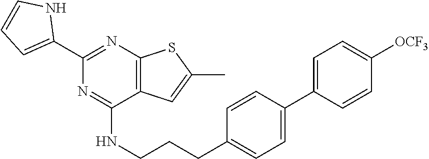 [SMILES: Cc1cc2c(NCCCc3ccc(-c4ccc(OC(F)(F)F)cc4)cc3)nc(-c3ccc[nH]3)nc2s1] | 433 | Cs₂CO₃ 2 h (80° C.) | 25% | 6-methyl-2-(1H- pyrrol-2-yl)-N-(3-(4′- (trifluoromethoxy)- [1,1′-biphenyl]-4- yl)propyl)thieno[2, 3-d]pyrimidin-4- amine |
| 568 | 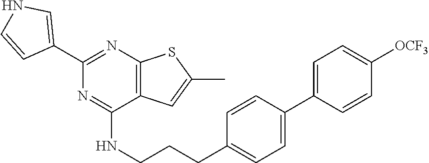 [SMILES: Cc1cc2c(NCCCc3ccc(-c4ccc(OC(F)(F)F)cc4)cc3)nc(-c3cc[nH]c3)nc2s1] | 433 | Cs₂CO₃ 1 h (80° C.) | 32% | 6-methyl-2-(1H- pyrrol-3-yl)-N-(3-(4′- (trifluoromethoxy)- [1,1′-biphenyl]-4- yl)propyl)thieno[2, 3-d]pyrimidin-4- amine |
| 569 | 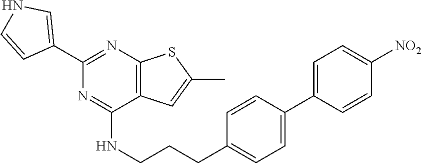 [SMILES: Cc1cc2c(NCCCc3ccc(-c4ccc([N+](=O)[O-])cc4)cc3)nc(-c3cc[nH]c3)nc2s1] | 472 | Cs₂CO₃ 1 h (90° C.) | 23% | 6-methyl-N-(3-(4′- nitro-[1,1′- biphenyl]-4- yl)propyl)-2-(1H- pyrrol-3- yl)thieno[2,3- d]pyrimidin-4- amine |
| 570 | 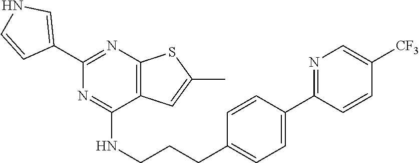 [SMILES: Cc1cc2c(NCCCc3ccc(-c4ccc(C(F)(F)F)cn4)cc3)nc(-c3cc[nH]c3)nc2s1] | 471 | Cs₂CO₃ 2 h (90° C.) | 42% | 6-methyl-2-(1H- pyrrol-3-yl)-N-(3- (4-(5- (trifluoromethyl) pyridin-2- yl)phenyl)propyl) thieno[2,3- d]pyrimidin-4- amine |
| 571 | 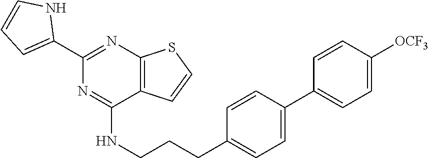 [SMILES: FC(F)(F)Oc1ccc(-c2ccc(CCCNc3nc(-c4ccc[nH]4)nc4sccc34)cc2)cc1] | 467 | Cs₂CO₃ 2 h (90° C.) | 28% | 2-(1H-pyrrol-2-yl)- N-(3-(4′- (trifluoromethoxy)- [1,1′-biphenyl]-4- yl)propyl)thieno[2, 3-d]pyrimidin-4- amine |
| 572 | 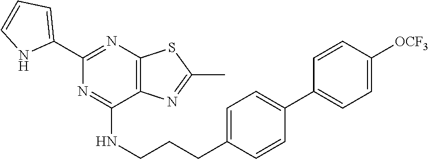 [SMILES: Cc1nc2c(NCCCc3ccc(-c4ccc(OC(F)(F)F)cc4)cc3)nc(-c3ccc[nH]3)nc2s1] | 463 | Cs₂CO₃ 2 h (90° C.) | 29% | 2-methyl-5-(1H- pyrrol-2-yl)-N-(3- (4′- (trifluoromethoxy)- [1,1′-biphenyl]-4- yl)propyl)thiazolo[ 5,4-d]pyrimidin-7- amine |

Examples from Scheme 10

| Cmpd. | |
|---|---|
| No. | Compound Data |
| 566 | ¹H NMR (400 MHz, |
| | CHLOROFORM-d) δ 9.26 (br. s., |
| | 1H), 7.12 − 7.20 (m, 1H), 6.94 − |
| | 7.07 (m, 3H), 6.79 (br. s., 1H), |
| | 6.71 (s, 1H), 6.26 − 6.30 (m, 1H), |
| | 5.48 (br. s., 1H), 2.98 − 3.04 (m, |
| | 1H), 2.51 (s, 3H), 2.10 (t, J = 7.63 |
| | Hz, 1H), 1.35 − 1.43 (m, 2H); |
| | LCMS: ESI [M + H]⁺ = 383.1 |
| 567 | ¹H NMR (399 MHz, |
| | METHANOL-d4) δ 7.64 (d, J = |
| | 8.17 Hz, 2H), 7.49 (d, J = 7.79 Hz, |
| | 2H), 7.24 − 7.35 (m, 4H), 6.96 (s, |
| | 1H), 6.89 − 6.93 (m, J = 1.95 Hz, |
| | 1H), 6.85 (s, 1H), 6.17 (br. s., 1H), |
| | 3.74 (t, J = 7.01 Hz, 2H), 2.79 (t, J = |
| | 7.40 Hz, 2H), 2.48 (s, 3H), 2.06 |
| | (quin, J = 6.91 Hz, 2H); LCMS: |
| | ESI [M + H]⁺ = 509.2 |
| 568 | ¹H NMR (399 MHz, |
| | METHANOL-d4) δ 7.66 (d, J = |
| | 8.56 Hz, 2H), 7.48 − 7.55 (m, 3H), |
| | 7.32 (d, J = 7.79 Hz, 4H), 6.98 (s, |
| | 1H), 6.75 (s, 2H), 3.71 (t, J = 7.01 |
| | Hz, 2H), 2.80 (t, J = 7.40 Hz, 2H), |
| | 2.51 (s, 3H), 2.11 (quin, J = 7.20 |
| | Hz, 2H); LCMS: ESI [M + H]⁺ = |
| | 509.1 |
| 569 | ¹H NMR (400 MHz, |
| | CHLOROFORM-d) δ 8.30 (d, J = |
| | 8.56 Hz, 2H), 7.73 (d, J = 8.56 Hz, |
| | 2H), 7.52 − 7.64 (m, 3H), 7.36 (d, J = |
| | 7.79 Hz, 2H), 6.93 (br. s., 1H), |
| | 6.80 (br. s., 1H), 6.60 (s, 1H), 4.90 |
| | (br. s., 1H), 3.78 (q, J = 6.23 Hz, |
| | 2H), 2.85 (t, J = 6.81 Hz, 2H), 2.49 |
| | (s, 3H), 2.06 − 2.19 (m, 2H); |
| | LCMS: ESI [M + H]⁺ = 470.1 |
| 570 | ¹H NMR (399 MHz, |
| | METHANOL-d4) δ 8.89 (s, 1H), |
| | 8.11 (d, J = 8.56 Hz, 1H), 7.91 − |
| | 7.99 (m, 3H), 7.47 (s, 1H), 7.36 (d, |
| | J = 7.79 Hz, 2H), 6.95 (s, 1H), 6.74 |
| | (d, J = 8.95 Hz, 2H), 3.68 (t, J = |
| | 6.81 Hz, 2H), 2.81 (t, J = 7.20 Hz, |
| | 2H), 2.48 (s, 3H), 2.09 (quin, J = |
| | 7.20 Hz, 2H); LCMS: ESI |
| | [M + H]⁺ = 494.1 |
| 571 | ¹H NMR (400 MHz, |
| | CHLOROFORM-d) δ 7.57 − 7.63 |
| | (m, 2H), 7.52 (d, J = 7.79 Hz, 2H), |
| | 7.30 (d, J = 8.17 Hz, 2H), 7.33 (d, |
| | J = 8.17 Hz, 2H), 7.03 − 7.13 (m, |
| | 2H), 6.95 (d, J = 5.45 Hz, 1H), |
| | 6.89 (br. s., 1H), 6.26 − 6.33 (m, |
| | 1H), 5.14 (br. s., 1H), 3.78 (q, J = |
| | 6.62 Hz, 2H), 2.85 (t, J = 7.20 Hz, |
| | 2H), 2.14 (quin, J = 7.10 Hz, 2H); |
| | LCMS: ESI [M + H]⁺ = 495.1 |
| 572 | ¹H NMR (399 MHz, |
| | METHANOL-d4) δ 7.65 (d, J = |
| | 8.56 Hz, 2H), 7.50 (d, J = 7.79 Hz, |
| | 2H), 7.31 (d, J = 8.17 Hz, 4H), |
| | 6.84 − 6.95 (m, 2H), 6.18 (t, J = |
| | 3.11 Hz, 1H), 3.77 (t, J = 6.62 Hz, |
| | 2H), 2.81 (t, J = 7.59 Hz, 2H), 2.72 |
| | (s, 3H), 1.99 − 2.16 (m, 2H); |

LCMS: ESI [M + H]⁺ = 510.1

### Example 13: Syntheses of Specific Compounds

### Experimental Procedure for the Preparation of Example 573

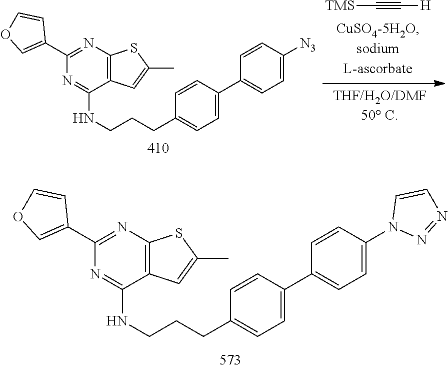 [SMILES: C#C[Si](C)(C)C ; Cc1cc2c(NCCCc3ccc(-c4ccc(N=[N+]=[N-])cc4)cc3)nc(-c3ccoc3)nc2s1 ; Cc1cc2c(NCCCc3ccc(-c4ccc(-n5ccnn5)cc4)cc3)nc(-c3ccoc3)nc2s1]

Following the General Click Protocol, example 410 (20.2 mg, 0.043 mmol) was reacted with (trimethylsilyl)acetylene (0.037 mL, 0.26 mmol) for 24 hours at 50° C., after which time the reaction was brought to room temperature, quenched with saturated Na₂S₂O₃(aq) and concentrated in vacuo. The crude reaction mixture was purified by silica gel column chromatography followed by prep HPLC to give 573: 20% yield; IUPAC: 2-(furan-3-yl)-6-methyl-N-(3-[4′-(1H-1,2,3-triazol-1-yl)-[1,1′-biphenyl]-4-yl]propyl)thieno[2,3-d]pyrimidin-4-amine; ¹H NMR (399 MHz, ACETONITRILE-d3) δ 8.34 (s, 1H), 8.09 (s, 1H), 7.87-7.92 (m, 2H), 7.79-7.86 (m, 3H), 7.63 (d, J=7.79 Hz, 2H), 7.51 (s, 1H), 7.37 (d, J=7.79 Hz, 2H), 6.94 (d, J=6.23 Hz, 2H), 6.17 (br. s., 1H), 3.67 (q, J=6.23 Hz, 2H), 2.81 (t, J=7.40 Hz, 2H), 2.52 (s, 3H), 2.04 (d, J=7.01 Hz, 2H); LCMS: ESI [M+H]⁺=493.2.

### Experimental Procedure for the Preparation of Examples 574 and 575

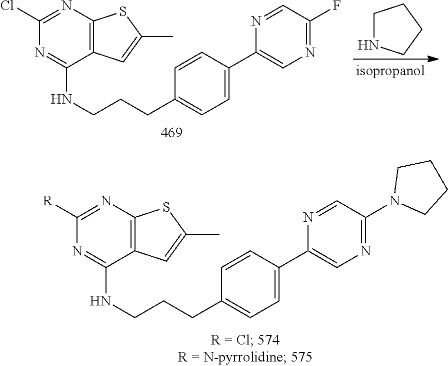

Example 469 (10.1 mg, 0.024 mmol) was dissolved into isopropanol (2.0 mL) and pyrrolidine (34.0 mg, 0.48 mmol) was added. The reaction was sealed and heated using either Conditions A (1 hour at 60° C.) or Conditions B (45 min, 150° C., microwave). Upon completion, the reaction was concentrated in vacuo and purified by prep HPLC to obtain the final product. Conditions A: 574: 55% yield; IUPAC: 2-(1H-pyrrol-2-yl)-N-(3-(4′-(trifluoromethoxy)-[1,1′-biphenyl]-4-yl)propyl)thieno[2,3-d]pyrimidin-4-amine; ¹H NMR (399 MHz, ACETONITRILE-d3) δ 8.48 (s, 1H), 7.93 (s, 1H), 7.77-7.84 (m, J=8.18 Hz, 2H), 7.25-7.31 (m, J=8.17 Hz, 2H), 6.91 (s, 1H), 6.44 (br. s., 1H), 3.44-3.55 (m, 6H), 2.73 (t, J=7.40 Hz, 2H), 2.48 (s, 3H), 1.97-2.04 (m, 6H); LCMS: ESI [M+H]⁺=465.2; Conditions B: 575: 59% yield; IUPAC: 6-methyl-2-(pyrrolidin-1-yl)-N-(3-(4-(5-(pyrrolidin-1-yl)pyrazin-2-yl)phenyl)propyl)thieno[2,3-d]pyrimidin-4-amine; ¹H NMR (399 MHz, METHANOL-d4) δ 8.43 (s, 1H), 7.95 (s, 1H), 7.71-7.78 (m, J=7.79 Hz, 2H), 7.24-7.31 (m, J=8.17 Hz, 2H), 6.80 (s, 1H), 3.49-3.60 (m, 6H), 3.44 (t, J=6.42 Hz, 4H), 2.74 (t, J=7.20 Hz, 2H), 2.41 (s, 3H), 1.97-2.12 (m, 6H), 1.90 (t, J=6.03 Hz, 4H); LCMS: ESI [M+H]⁺=500.3.

### Experimental Procedure for the Preparation of Example 576

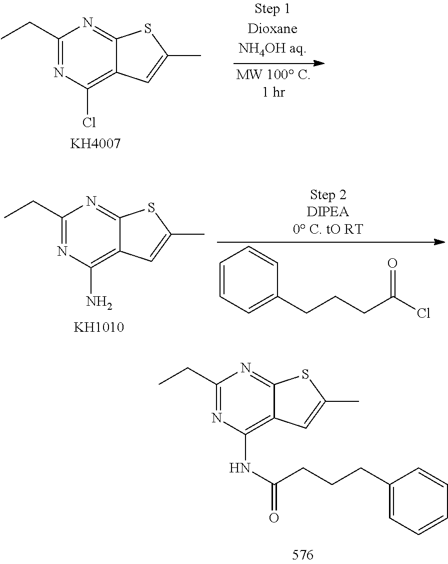

Step 1: Building block KH4007 (198 mg, 0.93 mmol) was dissolved in dioxane (1.5 mL) and 30% NH₄OH(aq) (3.0 mL, 15 mmol). The solution was reacted in a microwave at 100° C. for 1 hour. Upon completion, the reaction was concentrated in vacuo and purified by silica gel column chromatography to give intermediate KH1010: 54% yield; IUPAC: 2-ethyl-6-methylthieno[2,3-d]pyrimidin-4-amine; LCMS: ESI [M+H]⁺=194.2.

Step 2: KH1010 (20 mg, 0.103 mmol) was dissolved in anhydrous THF (0.25 mL) and DIPEA (22 μL) was added. This vial was cooled in an ice bath while a second vial with anhydrous DCM (1.0 mL) and 4-phenylbutyryl chloride (19 mg, 0.103 mmol) was prepared. The acid chloride in DCM solution was added dropwise to the first vial and allowed to come to room temperature overnight. After 16 hours the reaction was again cooled to 0° C. and another portion of acid chloride (19 mg, 0.103 mmol) was added. The reaction was again allowed to come to room temperature overnight, after which it was quenched with MeOH and purified by silica gel column chromatography to give 576: 26% yield; IUPAC: N-(2-ethyl-6-methylthieno[2,3-d]pyrimidin-4-yl)-4-phenylbutanamide; ¹H NMR (400 MHz, CHLOROFORM-d) δ 7.28-7.33 (m, 3H), 7.19-7.25 (m, 3H), 2.95 (q, J=7.69 Hz, 2H), 2.76 (t, J=7.43 Hz, 2H), 2.71 (br. s., 2H), 2.61 (s, 3H), 2.13 (quin, J=7.43 Hz, 2H), 1.38 (t, J=7.63 Hz, 3H); LCMS: ESI [M+H]⁺=340.2.

### Experimental Procedure for the Preparation of Example 577

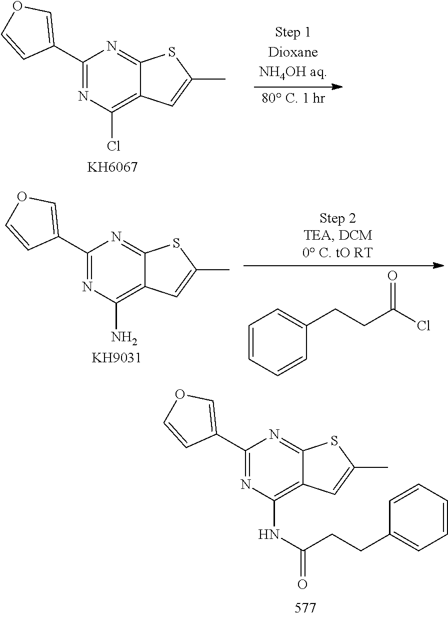

Step 1: Building block KH6067 (62 mg, 0.247 mmol) was dissolved in dioxane (1.0 mL) and 30% NH₄OH(aq) (2.0 mL, 10 mmol). The solution was reacted in a pressure vessel at 80° C. for 1 hour. Upon completion, the reaction was concentrated in vacuo and purified by silica gel column chromatography to give intermediate KH9031: 88% yield; IUPAC: 2-(furan-3-yl)-6-methylthieno[2,3-d]pyrimidin-4-amine; LCMS: ESI [M+H]⁺=232.1.

Step 2: KH9031 (15 mg, 0.065 mmol) was dissolved in anhydrous DCM (2.0 mL) and TEA (7.9 mg, 0.078 mmol) was added. The reaction was cooled on an ice bath and hydrocinnamoyl chloride (12.1 mg, 0.072 mmol) was added. The reaction was allowed to come to room temperature overnight. After 16 hours the reaction was again cooled to 0° C. and another portion of acid chloride (22 mg, 0.130 mmol) was added. The reaction was again allowed to come to room temperature overnight, after which it was quenched with MeOH and purified by silica gel column chromatography followed by a second purification in a prep HPLC to give 577: 4% yield; IUPAC: N-(2-(furan-3-yl)-6-methylthieno[2,3-d]pyrimidin-4-yl)-3-phenylpropanamide; ¹H NMR (399 MHz, METHANOL-d4) δ 8.55 (s, 1H), 8.25 (s, 1H), 7.59 (s, 1H), 7.25-7.35 (m, 3H), 7.22 (br. s., 1H), 7.07 (s, 1H), 6.92 (s, 1H), 3.07 (t, J=7.40 Hz, 2H), 2.97 (t, J=7.20 Hz, 2H), 2.59 (s, 3H); LCMS: ESI [M+H]⁺=364.1; LCMS: ESI [M+H]+=364.1.

### Experimental Procedure for the Preparation of Example 578

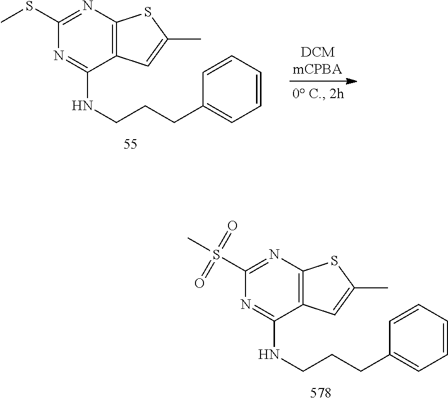 [SMILES: CSc1nc(NCCCc2ccccc2)c2cc(C)sc2n1 ; Cc1cc2c(NCCCc3ccccc3)nc(S(C)(=O)=O)nc2s1]

Example 55 (13.0 mg, 0.04 mmol) was dissolved in anhydrous DCM (1.0 mL) and the reaction was cooled to 0° C. before slowly adding mCPBA (27.6 mg, 0.16 mmol) with rapid stirring. After 2 hours, DCM was added and the organic layer was washed with saturated NaHCO₃(aq) (3×) before drying to a solid in vacuo. The product was purified on a prep HPLC to obtain 578: 50% yield; IUPAC: 6-methyl-2-(methylsulfonyl)-N-(3-phenylpropyl)thieno[2,3-d]pyrimidin-4-amine; ¹H NMR (400 MHz, CHLOROFORM-d) δ 7.29-7.34 (m, 2H), 7.19-7.25 (m, 3H), 6.66 (s, 1H), 5.37 (br. s., 1H), 3.69 (q, J=5.61 Hz, 2H), 3.27 (s, 3H), 2.76 (t, J=7.24 Hz, 2H), 2.58 (s, 3H), 2.00-2.09 (m, 2H); LCMS: ESI [M+H]⁺=362.1.

### Experimental Procedure for the Preparation of Example 579

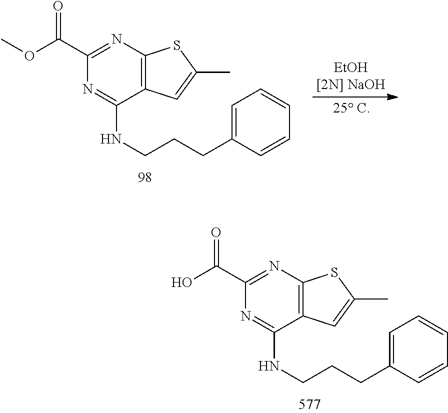 [SMILES: COC(=O)c1nc(NCCCc2ccccc2)c2cc(C)sc2n1 ; Cc1cc2c(NCCCc3ccccc3)nc(C(=O)O)nc2s1]

Example 98 (8.6 mg, 0.025 mmol) was added to a vial along with EtOH (0.5 mL) and [2N] NaOH(aq) (0.5 mL). Reaction was complete after 20 minutes at room temperature and was dried to a solid in vacuo. Added DI water (1.0 mL) and stirred rapidly while [4N] HCl(aq) was added dropwise until product precipitated. The product was extracted into DCM (3×) before being dried under high vacuum to give 579: 72% yield; IUPAC: 6-methyl-4-((3-phenylpropyl)amino)thieno[2,3-d]pyrimidine-2-carboxylic acid; ¹H NMR (400 MHz, CHLOROFORM-d) δ 6.81-6.88 (m, 2H), 6.73-6.79 (m, 3H), 6.47 (s, 1H), 3.24 (t, J=7.04 Hz, 2H), 2.90-2.94 (m, J=1.57 Hz, 1H), 2.31 (t, J=7.43 Hz, 2H), 2.15 (s, 3H), 1.60 (quin, J=7.43 Hz, 2H); LCMS: ESI [M+H]⁺=328.1.

### Experimental Procedure for the Preparation of Example 580

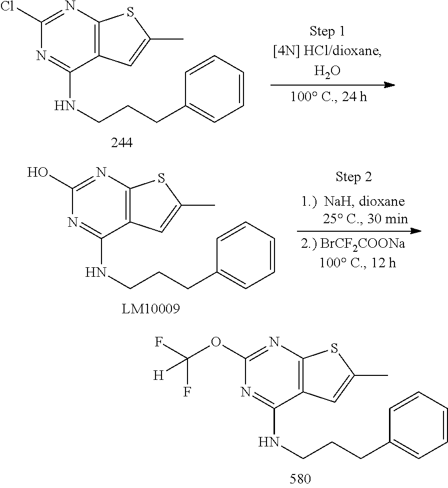 [SMILES: Cc1cc2c(NCCCc3ccccc3)nc(Cl)nc2s1 ; Cc1cc2c(NCCCc3ccccc3)nc(O)nc2s1 ; Cc1cc2c(NCCCc3ccccc3)nc(OC(F)F)nc2s1]

Step 1: Example 224 (0.20 mmol) was added to a threaded pressure vial, followed by [4N] HCl/Dioxane(aq) (2 mL) and H₂O (0.2 mL), and the reaction was sealed and stirred at 100° C. for 20 h. Upon completion, the reaction was cooled to rt and neutralized with [1N] NaOH(aq). The solids were filtered, concentrated in vacuo, and purified by silica gel column chromatography, to give the pure intermediate LM10009: 45% yield; IUPAC: 6-methyl-4-((3-phenylpropyl)amino)thieno[2,3-d]pyrimidin-2-ol; LCMS: ESI [M+H]⁺=300.1.

Step 2: Intermediate LM10009 (14.2 mg, 0.047 mmol) was added to a pressure vessel. Anhydrous dioxane (1.0 mL) was added along with NaH (2.1 mg, 0.052 mmol) and the reaction was stirred at room temperature for 30 minutes. Sodium bromodifluoroacetate (10.2 mg, 0.052 mmol) was added and the reaction was heated at 60° C. There was no sign of product after 1 hour, so the temperature was increased to 80° C. and the reaction was run overnight. Upon completion, the reaction was concentrated in vacuo, and purified by silica gel column chromatography to give 580: 25% yield; IUPAC: 2-(difluoromethoxy)-6-methyl-N-(3-phenylpropyl)thieno[2,3-d]pyrimidin-4-amine; ¹H NMR (400 MHz, CHLOROFORM-d) δ 7.82 (br. t, J=60.30 Hz, 1H), 7.29-7.35 (m, 2H), 7.20-7.26 (m, 3H), 6.38 (s, 1H), 5.42 (br. s., 1H), 3.70 (q, J=6.13 Hz, 2H), 2.75 (t, J=7.24 Hz, 2H), 2.46 (s, 3H), 2.03 (quin, J=7.14 Hz, 2H); LCMS: ESI [M+H]⁺=350.1.

### Experimental Procedure for the Preparation of Example 581

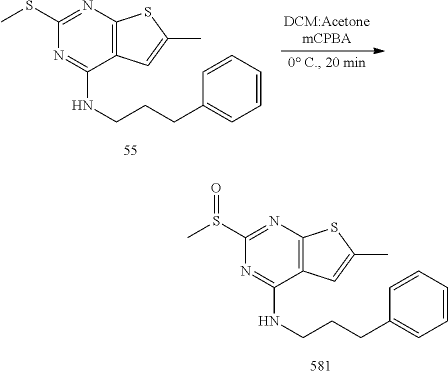 [SMILES: CSc1nc(NCCCc2ccccc2)c2cc(C)sc2n1 ; Cc1cc2c(NCCCc3ccccc3)nc(S(C)=O)nc2s1]

Example 55 (12.6 mg, 0.038 mmol) was added to a vial followed by anhydrous DCM (0.75 mL) and acetone (0.75 mL). The reaction was cooled on an ice bath before slowly adding mCPBA (13.1 mg, 0.076 mmol) with rapid stirring. After 20 minutes at 0° C., the reaction was complete and was dried to a solid in vacuo before purifying by prep HPLC to obtain 581: 21% yield; IUPAC: 6-methyl-2-(methylsulfinyl)-N-(3-phenylpropyl)thieno[2,3-d]pyrimidin-4-amine; ¹H NMR (400 MHz, CHLOROFORM-d) δ 7.31 (d, J=6.65 Hz, 2H), 7.19-7.24 (m, 3H), 6.63 (br. s., 1H), 5.23 (br. s., 1H), 3.68 (q, J=6.26 Hz, 2H), 2.90 (s, 3H), 2.77 (t, J=7.04 Hz, 2H), 2.57 (s, 3H), 2.04 (quin, J=7.14 Hz, 2H); LCMS: ESI [M+H]⁺=346.0.

### Experimental Procedure for the Preparation of Example 582

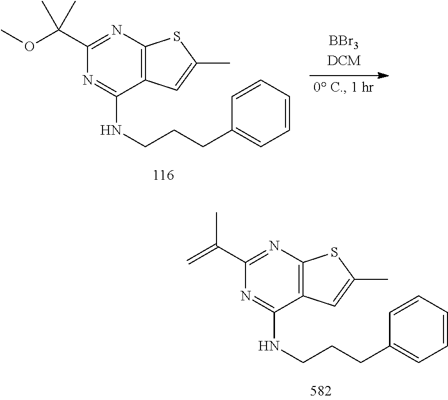 [SMILES: COC(C)(C)c1nc(NCCCc2ccccc2)c2cc(C)sc2n1 ; C=C(C)c1nc(NCCCc2ccccc2)c2cc(C)sc2n1]

Example 116 (8.9 mg, 0.025 mmol) was added to a vial along with anhydrous DCM (1.0 mL). The reaction was cooled on an ice bath before BBr₃ (7.5 mg, 0.03 mmol) was added dropwise. After 30 minutes, the reaction was quenched with DI water (2.0 mL) and stirred overnight at room temperature. The reaction was dried to a solid in vacuo before purifying by prep HPLC to obtain 582: 40% yield; IUPAC: 6-methyl-N-(3-phenylpropyl)-2-(prop-1-en-2-yl)thieno[2,3-d]pyrimidin-4-amine; ¹H NMR (400 MHz, CHLOROFORM-d) δ 7.28-7.34 (m, 2H), 7.19-7.25 (m, J=7.04 Hz, 3H), 6.61 (s, 1H), 6.33 (br. s., 1H), 5.41 (br. s., 1H), 4.86 (br. s., 1H), 3.66-3.73 (m, 2H), 2.76 (t, J=7.43 Hz, 2H), 2.55 (s, 3H), 2.24 (s, 3H), 2.02-2.10 (m, 2H); LCMS: ESI [M+H]⁺=324.2.

### Experimental Procedure for the Preparation of Example 583

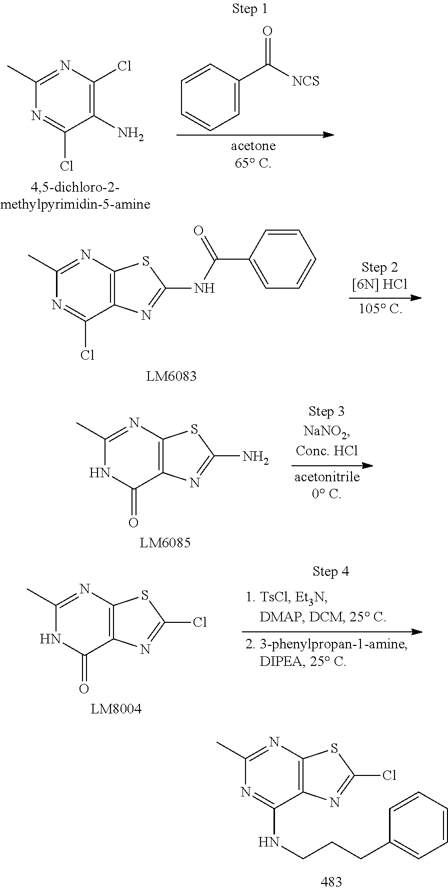

Step 1: 4,6-dichloro-2-methylpyrimidin-5-amine (1.0 g, 5.61 mmol) and benzoyl isothiocyanate (0.92 g, 5.61 mmol) were dissolved in acetone (10 mL) in a pressure vial. The reaction was sealed and stirred for 2 hours at 65° C. Upon completion the reaction was concentrated under reduced pressure and dried overnight under high vacuum to give the intermediate LM6083: 88% yield; IUPAC: N-(7-chloro-5-methylthiazolo[5,4-d]pyrimidin-2-yl)benzamide; LCMS: ESI [M+H]⁺=305.0.

Step 2: Intermediate LM6083 (1.0 g, 3.28 mmol) was dissolved in to [6N] HCl(aq) (70 mL) and heated under a reflux condenser for 2 days at 105° C., after which time the reaction was concentrated to half the original volume and neutralized carefully with saturated NaHCO₃(aq). The precipitated product was filtered and washed with DI water (3×) and dried overnight under high vacuum to give an inseparable ˜2:3 mixture of products: the undesired 2-hydroxy-derivative and the desired 2-amino-intermediate LM6085: 266 mg (with 40% impurity); IUPAC: 2-amino-5-methylthiazolo[5,4-d]pyrimidin-7(6H)-one; LCMS: ESI [M+H]⁺=183.1.

Step 3: NaNO₂ (75.7 mg, 1.1 mmol) was dissolved in a minimal amount of DI water and added slowly dropwise to a separate flask containing intermediate LM6085 (100 mg, 40% impure) in acetonitrile (5.0 mL) at 0° C., taking care not to cause the temperature to rise. The reaction was stirred for 8 hours at 0° C., then placed in a freezer for 16 hours, after which the unwanted solids were filtered from the reaction mixture. The reaction was again cooled to 0° C. and neutralized with saturated NaHCO₃(aq). The crude reaction mixture was concentrated in vacuo then dissolved in DMSO. The insoluble solids were filtered, and the filtrate was directly purified by prep HPLC to give intermediate LM8004: 510% yield (based on 60% of the starting material); IUPAC: 2-chloro-5-methylthiazolo[5,4-d]pyrimidin-7(6H)-one; LCMS: ESI [M+H]⁺=202.0.

Step 4: Intermediate LM8004 (4.5 mg, 0.022 mmol) was dissolved in DCM (1 mL) at room temperature. p-Toluenesulfonyl chloride (6.3 mg, 0.033 mmol) was added, followed by triethylamine (5 μL, 0.038 mmol) and DMAP (0.5 mg) and the reaction was stirred for 16 hours. Once the starting material had completely converted into the tosylate intermediate, 3-phenylpropan-1-amine (0.1 mL) and DIPEA (0.1 mL) were added, and the reaction was stirred for 1 hour at room temperature. Upon completion, the reaction was concentrated in vacuo and purified by silica gel column chromatography to give the desired 583: 34% yield; IUPAC: 2-chloro-5-methyl-N-(3-phenylpropyl)thiazolo[5,4-d]pyrimidin-7-amine; ¹H NMR (400 MHz, METHANOL-d₄) δ 7.08-7.16 (m, 4H), 7.04 (d, J=6.65 Hz, 1H), 3.50 (t, J=6.65 Hz, 2H), 2.61 (t, J=7.63 Hz, 2H), 2.40 (s, 3H), 1.90 (t, J=7.43 Hz, 2H); LCMS: ESI [M+H]+=319.0.

### Experimental Procedure for the Preparation of Example 584

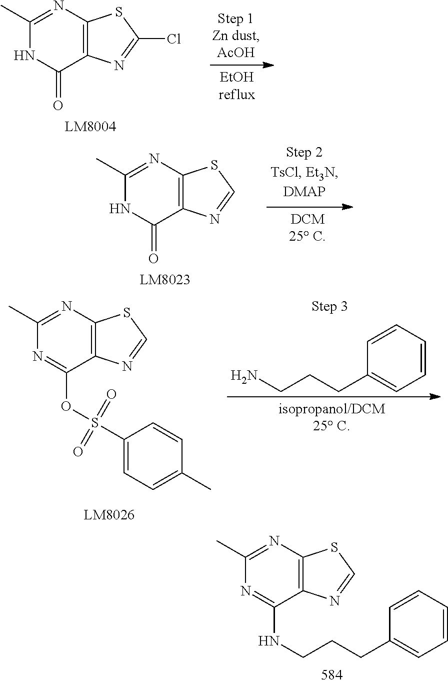

Step 1: Starting material LM8004 was prepared as described in the synthesis of example 583. LM8004 (20 mg, 0.10 mmol) was added to EtOH (0.5 mL), followed by zinc dust (19.5 mg, 0.30 mmol) and acetic acid (0.1 mL). The reaction was refluxed for 4 hours, then stirred at room temperature for an additional 16 hours. The solids were filtered, and the filtrate was concentrated, then dissolved in DCM and washed with saturated NaHCO₃(aq) (3×). The DCM layer was concentrated in vacuo, and purified by prep HPLC, to give intermediate LM8023: 36% yield; IUPAC: 5-methylthiazolo[5,4-d]pyrimidin-7(6H)-one; LCMS: ESI [M+H]⁺=168.0.

Step 2: LM8023 (6.0 mg, 0.036 mmol) was dissolved in DCM (1 mL) at room temperature. p-Toluenesulfonyl chloride (13.7 mg, 0.072 mmol) was added, followed by triethylamine (11 μL, 0.079 mmol) and DMAP (<0.01 mg) and the reaction was stirred for 2.5 hours at room temperature. Upon completion, the reaction contents were concentrated in vacuo at room temperature and purified by column chromatography. The pure fractions were also concentrated at room temperature to give the tosylated intermediate LM8026: 45% yield; IUPAC: 5-methylthiazolo[5,4-d]pyrimidin-7-yl 4-methylbenzenesulfonate; LCMS: ESI [M+H]⁺=322.0.

Step 3: LM8026 (5.2 mg, 0.016 mmol) was dissolved in isopropanol (0.9 mL) and DCM (0.1 mL), 3-phenylpropan-1-amine (10 μL, 0.072 mmol) was added and the reaction was stirred at room temperature for 18 hours. Upon completion, the reaction was concentrated in vacuo and purified by column chromatography to give the pure 584: 44% yield; IUPAC: 5-methyl-N-(3-phenylpropyl)thiazolo[5,4-d]pyrimidin-7-amine; ¹H NMR (400 MHz, METHANOL-d₄) δ 8.90 (s, 1H), 7.18-7.25 (m, 4H), 7.12 (d, J=6.65 Hz, 1H), 3.61 (t, J=6.85 Hz, 2H), 2.71 (t, J=7.63 Hz, 2H), 2.51 (s, 3H), 2.00 (quin, J=7.34 Hz, 2H); LCMS: ESI [M+H]⁺=285.1.

### Experimental Procedure for the Preparation of Example 586

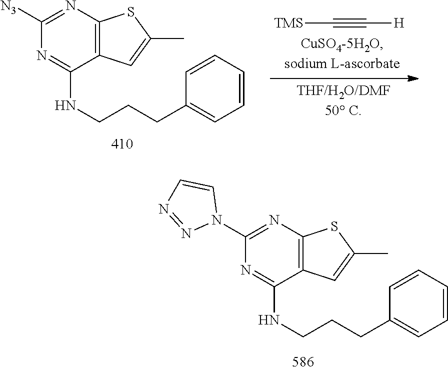 [SMILES: Cc1cc2c(NCCCc3ccccc3)nc(N=[N+]=[N-])nc2s1 ; C#C[Si](C)(C)C ; Cc1cc2c(NCCCc3ccccc3)nc(-n3ccnn3)nc2s1]

Following the General Click Protocol, example 156 (8.4 mg, 0.019 mmol) was reacted with (trimethylsilyl)acetylene (0.016 mL, 0.12 mmol) for 24 hours at 50° C., after which time the reaction was brought to room temperature and quenched with saturated Na₂S₂O₃(aq). The entire reaction contents were evaporated, and the crude material was purified by silica gel column chromatography to give the pure 586: 26% yield; IUPAC: 6-methyl-N-(3-phenylpropyl)-2-(1H-1,2,3-triazol-1-yl)thieno[2,3-d]pyrimidin-4-amine; ¹H NMR (400 MHz, CHLOROFORM-d) δ 8.42 (t, J=1.2 Hz, 1H), 7.78 (t, J=1.1 Hz, 1H), 7.32 (td, J=7.0, 1.5 Hz, 2H), 7.23 (d, J=7.6 Hz, 3H), 6.66-6.61 (m, 1H), 5.25 (s, 1H), 3.74 (q, J=6.6 Hz, 2H), 2.79 (t, J=7.2 Hz, 2H), 2.57 (t, J=1.2 Hz, 3H), 2.08 (p, J=7.1 Hz, 2H); LCMS: ESI [M+H]+=351.1.

### Experimental Procedure for the Preparation of Example 587

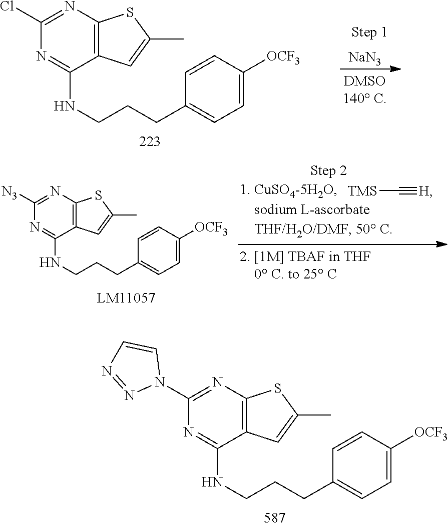

Step 1: To a threaded pressure vial was added DMSO (1 mL) and example 223 (31 mg, 0.08 mmol). Sodium azide was added (12.5 mg, 0.19 mmol) was added, and the reaction was sealed and stirred for 27 hours at 140° C. At this time, decomposition was starting to occur, and so the incomplete reaction was cooled to room temperature. DI water was added, and the precipitated product was filtered and rinsed well with DI water (3×); this process was repeated. The solids were dried overnight under high vacuum to give LM11057: 63% yield; 2-azido-6-methyl-N-(3-(4-(trifluoromethoxy)phenyl)propyl)thieno[2,3-d]pyrimidin-4-amine; LCMS: ESI [M+H]⁺=409.1.

Step 2: Following the General Click Protocol, LM11057 (20.4 mg, 0.05 mmol) was reacted with (trimethylsilyl)acetylene (29.0 mg, 0.30 mmol) for 30 hours at 50° C., 50° C., after which time the reaction was brought to room temperature. DI water was added to precipitate the product, which was filtered and rinsed well with DI water; this process was repeated. As the solids were found to contain a mix of both the desired product and the trimethylsilated-product, the solids were redissolved in THF (2 mL) and cooled to 0° C. [1M] Tetrabutylammonium fluoride in THF (0.2 mL, 0.2 mmol) was added, and the reaction was brought to room temperature and stirred overnight. Upon completion, the reaction contents were evaporated and DCM was added, and the organic layer was washed with [1N] HCl(aq) (3×). The DCM layer was concentrated in vacuo, and purified by silica gel column chromatography followed by prep HPLC to give 587: 14% yield; IUPAC: 6-methyl-2-(1H-1,2,3-triazol-1-yl)-N-(3-(4-(trifluoromethoxy)phenyl)propyl)thieno[2,3-d]pyrimidin-4-amine; ¹H NMR (400 MHz, CHLOROFORM-d) δ 8.45 (s, 1H), 7.79 (s, 1H), 7.29-7.20 (m, 2H), 7.15 (d, J=8.2 Hz, 2H), 6.71 (d, J=2.6 Hz, 1H), 5.30 (s, 1H), 3.74 (d, J=6.8 Hz, 2H), 2.78 (t, J=7.6 Hz, 2H), 2.57 (d, J=2.6 Hz, 3H), 2.11-2.01 (m, 2H); LCMS: ESI [M+H]⁺=435.1.

### Experimental Procedure for the Preparation of Examples 588 and 589

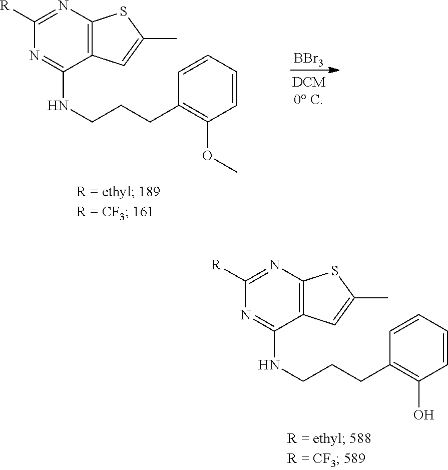

The methoxyphenyl derivative 189 or 161 (0.026 mmol) was dissolved in DCM (1 mL) and the reaction was cooled to 0° C. BBr₃ (3 eqv.) was added, and the reaction was stirred for 30 min at 0° C. Upon completion, the reaction was concentrated in vacuo, and subsequently co-evaporated with MeOH (3×). The crude residue was then purified by silica gel column chromatography, followed by prep HPLC to give the desired phenol. Example 588: 36% yield; IUPAC: 2-(3-((2-ethyl-6-methylthieno[2,3-d]pyrimidin-4-yl)amino)propyl)phenol; ¹H NMR (400 MHz, CHLOROFORM-d) δ 7.16 (dt, J=7.4, 2.2 Hz, 1H), 7.10 (tt, J=7.6, 2.3 Hz, 1H), 6.88 (tdd, J=8.1, 6.2, 1.9 Hz, 2H), 6.60 (s, 1H), 6.02 (s, 1H), 3.64 (q, J=5.9 Hz, 2H), 2.90-2.77 (m, 4H), 2.40 (d, J=2.2 Hz, 3H), 2.00 (tq, J=7.0, 3.5, 2.2 Hz, 2H), 1.39-1.30 (m, 3H); LCMS: ESI [M+H]⁺=328.2. Example 589: 57% yield; IUPAC: 2-(3-((6-methyl-2-(trifluoromethyl)thieno[2,3-d]pyrimidin-4-yl)amino)propyl)phenol; ¹H NMR (400 MHz, CHLOROFORM-d) δ 7.22-7.15 (m, 1H), 7.11 (d, J=8.0 Hz, 1H), 6.93 (d, J=7.7 Hz, 1H), 6.79 (d, J=7.9 Hz, 1H), 6.67 (s, 1H), 5.65 (s, 1H), 5.16 (s, 1H), 3.66 (q, J=6.2 Hz, 2H), 2.79 (t, J=7.2 Hz, 2H), 2.56 (s, 3H), 2.06-1.98 (m, 2H); LCMS: ESI [M+H]⁺=368.1.

### Experimental Procedure for the Preparation of Example 590

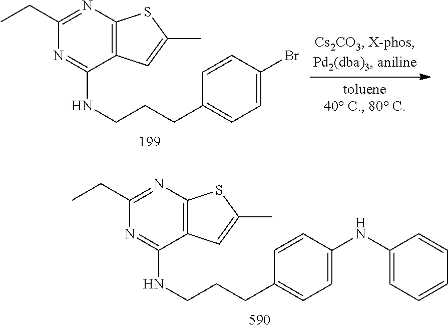 [SMILES: CCc1nc(NCCCc2ccc(Br)cc2)c2cc(C)sc2n1 ; CCc1nc(NCCCc2ccc(Nc3ccccc3)cc2)c2cc(C)sc2n1]

Anhydrous toluene (0.5 mL) was added to a flask containing X-Phos (2.4 mg, 0.05 mmol) and Pd₂(dba)₃ (1.0 mg, 0.001 mmol). The reaction was flushed/purged 3× with N₂, and kept under an atmosphere of N₂ thereafter. The reagents were stirred for 15 minutes at 40° C., after which time they were transferred via cannula to a separate flask (also under an atmosphere of N₂) containing example 199 (20 mg, 0.51 mmol), aniline (9.54 mg, 0.10 mmol) and Cs₂CO₃ (50 mg, 0.15 mmol). The reaction was stirred at for 24 hours at 80° C., after which time the reaction was cooled to room temperature and concentrated in vacuo. The reaction was purified by silica gel column chromatography, then prep HPLC to give 590: 41% yield; IUPAC: 2-ethyl-6-methyl-N-(3-(4-(phenylamino)phenyl)propyl)thieno[2,3-d]pyrimidin-4-amine; 1H NMR (400 MHz, CHLOROFORM-d) δ 7.29-7.21 (m, 2H), 7.13 (dd, J=8.5, 2.4 Hz, 2H), 7.04 (dt, J=8.5, 2.1 Hz, 4H), 6.96-6.85 (m, 1H), 6.59 (d, J=2.8 Hz, 1H), 5.75 (br s, 1H), 5.42 (br s, 1H), 3.67 (qd, J=6.7, 2.2 Hz, 2H), 2.84 (qd, J=7.6, 2.3 Hz, 2H), 2.71 (td, J=7.4, 2.3 Hz, 2H), 2.49 (dd, J=2.5, 1.3 Hz, 3H), 2.02 (pd, J=7.0, 2.2 Hz, 2H), 1.35 (td, J=7.6, 2.3 Hz, 3H); LCMS: ESI [M+H]⁺=403.2.

### Experimental Procedure for the Preparation of Examples 591, 592, 593

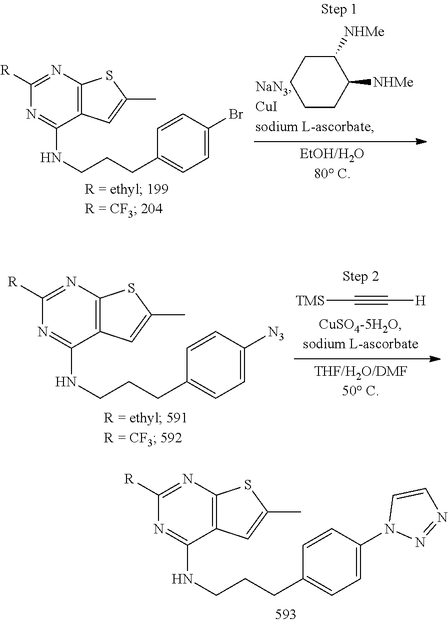

Step 1: The starting material, either example 199 or 204 (0.51 mmol), was dissolved into EtOH (0.5 mL) and DI water (0.06 mL). (1R,2R)—N,N-dimethylcyclohexane-1,2-diamine (0.3 eqv.), NaN₃ (1.5 eqv.), CuI (0.2 eqv.) and sodium L-ascorbate (0.1 eqv.) were added sequentially to the reaction, and the reaction was flushed/purged (3×) with N₂, after which the N₂ atmosphere was maintained. The reaction was then stirred for 1 hour at 80° C. Upon completion, the reaction was cooled to room temperature, and stirred in a mixture of saturated NH₄Cl(aq) and EtOAc (1:1 ratio, 5 mL) for 1 hour, after which the layers were separated and the aqueous layer was extracted with EtOAc (3×). The EtOAc fractions were combined and concentrated in vacuo and the reaction was purified first by silica gel column chromatography, then by prep HPLC, to give the aryl azide product. Example 591: 78% yield; IUPAC: N-(3-(4-azidophenyl)propyl)-2-ethyl-6-methylthieno[2,3-d]pyrimidin-4-amine; ¹H NMR (400 MHz, CHLOROFORM-d) δ 7.23-7.16 (m, 2H), 6.99-6.92 (m, 2H), 6.59 (d, J=1.5 Hz, 1H), 4.86 (s, 1H), 3.66 (q, J=6.5 Hz, 2H), 2.88-2.77 (m, 2H), 2.73 (t, J=7.4 Hz, 2H), 2.53 (d, J=1.2 Hz, 3H), 2.06-1.94 (m, 2H), 1.34 (td, J=7.5, 1.3 Hz, 3H); LCMS: ESI [M+H]+=353.2. Example 592: 51% yield; IUPAC: N-(3-(4-azidophenyl)propyl)-6-methyl-2-(trifluoromethyl)thieno[2,3-d]pyrimidin-4-amine; ¹H NMR (400 MHz, CHLOROFORM-d) δ 7.22-7.16 (m, 2H), 6.95 (dq, J=8.5, 2.3, 1.7 Hz, 2H), 6.66 (dt, J=3.0, 1.4 Hz, 1H), 5.11 (s, 1H), 3.69 (q, J=5.9, 5.1 Hz, 2H), 2.78-2.68 (m, 2H), 2.59 (t, J=2.2 Hz, 3H), 2.08-1.96 (m, 2H); LCMS: ESI [M+H]⁺=393.1.

Step 2: Following the General Click Protocol, example 591 (10 mg, 0.028 mmol) and (trimethylsilyl)acetylene (17 mg, 0.17 mmol) were reacted for 24 hours at 50° C., after which time the reaction was brought to room temperature and quenched with saturated Na₂S₂O₃(aq). The reaction contents were evaporated, and the crude material was purified by silica gel column chromatography then prep HPLC, to give the pure 593: 25% yield; IUPAC: N-(3-(4-(1H-1,2,3-triazol-1-yl)phenyl)propyl)-2-ethyl-6-methylthieno[2,3-d]pyrimidin-4-amine; ¹H NMR (400 MHz, CHLOROFORM-d) δ 7.95 (s, 1H), 7.85 (s, 1H), 7.65 (d, J=7.9 Hz, 2H), 7.37 (d, J=8.0 Hz, 2H), 6.61 (s, 1H), 4.90 (s, 1H), 3.70 (q, J=6.8 Hz, 2H), 2.83 (q, J=7.6 Hz, 4H), 2.49 (s, 3H), 2.06 (q, J=7.3 Hz, 2H), 1.38-1.30 (m, 3H); LCMS: ESI [M+H]+=379.2.

### Experimental Procedure for the Preparation of Examples 594 and 595

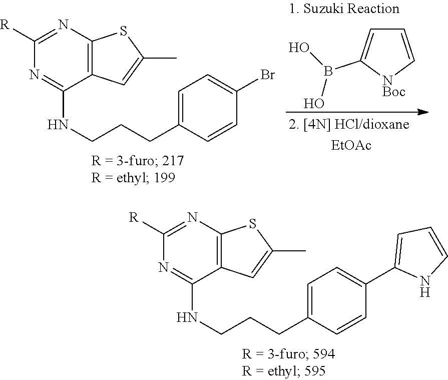

Following the General Suzuki Protocol, example 217 or 199 (0.047 mmol) was reacted with the boc-protected 2-pyrrole boronic acid (19.7 mg, 0.93 mmol), Cs₂CO₃ (38.0 mg, 0.12 mmol) and Pd(PPh₃)₄ (8.1 mg, 0.007 mmol) for 1.5 hours at 80° C. The boc-protected intermediate was purified by silica gel column chromatography then dissolved in EtOAc (5 mL) and [4N] HCl(aq) (5 mL) was slowly added. The reaction was allowed to stir for 2 days at room temperature. Upon completion, the reaction was concentrated and purified by silica gel column chromatography, then prep HPLC to give desired compounds, respectively. Example 594: 4% yield; IUPAC: 2-(furan-3-yl)-6-methyl-N-(3-[4-(1H-pyrrol-2-yl)phenyl]propyl)thieno[2,3-d]pyrimidin-4-amine; ¹H NMR (399 MHz, METHANOL-d4) δ 8.10 (s, 1H), 7.42-7.51 (m, 3H), 7.20 (d, J=8.17 Hz, 2H), 6.99 (s, 1H), 6.90 (s, 1H), 6.78 (s, 1H), 6.42 (d, J=3.11 Hz, 1H), 6.14 (br. s., 1H), 3.67 (t, J=7.20 Hz, 2H), 2.74 (t, J=7.20 Hz, 2H), 2.51 (s, 3H), 1.97-2.10 (m, 2H); LCMS: ESI [M+H]⁺=415.2. Example 595: 8% yield; IUPAC: N-(3-(4-(1H-pyrrol-2-yl)phenyl)propyl)-2-ethyl-6-methylthieno[2,3-d]pyrimidin-4-amine; ¹H NMR (400 MHz, METHANOL-d4) δ 7.33 (d, J=8.22 Hz, 2H), 7.06 (d, J=8.22 Hz, 2H), 6.92 (d, J=0.78 Hz, 1H), 6.67 (d, J=1.17 Hz, 1H), 6.29 (br. s., 1H), 6.03 (d, J=3.13 Hz, 1H), 3.58 (t, J=7.04 Hz, 2H), 2.59-2.73 (m, 4H), 2.38-2.45 (m, 3H), 1.89-2.04 (m, 2H), 1.20-1.26 (m, 3H); LCMS: ESI [M+H]⁺=377.2.

### Experimental Procedure for the Preparation of Example 596

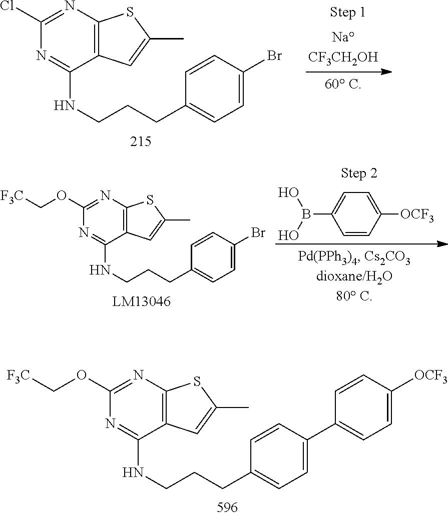

Step 1: To a threaded pressure vial containing 2,2,2-trifluoroethanol (1.0 mL) was carefully added sodium metal (46 mg, 2.0 mmol). After the sodium was fully consumed, example 215 (17.0 mg, 0.043 mmol) was added and the vial was sealed and heated to 110° C. for 24 hours. Upon completion, the reaction was cooled to room temperature and concentrated in vacuo. The crude reaction mixture was purified by silica gel column chromatography to give the intermediate LM13046: 76% yield; IUPAC: N-(3-(4-bromophenyl)propyl)-6-methyl-2-(2,2,2-trifluoroethoxy)thieno[2,3-d]pyrimidin-4-amine; LCMS: ESI [M+H]⁺=460.1, 462.1.

Step 2: Following the General Suzuki Protocol, intermediate LM13046 (15 mg, 0.033 mmol) was reacted with 4-trifluoromethoxyphenylboronic acid (13.4 mg, 0.065 mmol), Cs₂CO₃ (26.5 mg, 0.081 mmol), and Pd(PPh₃)₄ (5.6 mg, 0.005 mmol) for 2 hours at 80° C. The crude reaction residue was purified by silica gel column chromatography then prep HPLC to give the desired compound 596: 60% yield; IUPAC: 6-methyl-2-(2,2,2-trifluoroethoxy)-N-(3-(4′-(trifluoromethoxy)-[1,1′-biphenyl]-4-yl)propyl)thieno[2,3-d]pyrimidin-4-amine; ¹H NMR (400 MHz, CHLOROFORM-d) δ 7.54-7.64 (m, 2H), 7.49 (br. s., 2H), 7.21-7.32 (m, 4H), 6.42 (br. s., 1H), 4.95 (br. s., 1H), 4.67-4.81 (m, 2H), 3.60-3.72 (m, 2H), 2.79 (d, J 6.65 Hz, 2H), 2.38 (d, J 4.70 Hz, 3H), 2.05 (d, J=6.65 Hz, 2H); LCMS: ESI [M+H]⁺=542.2.

### Experimental Procedure for the Preparation of Example 597

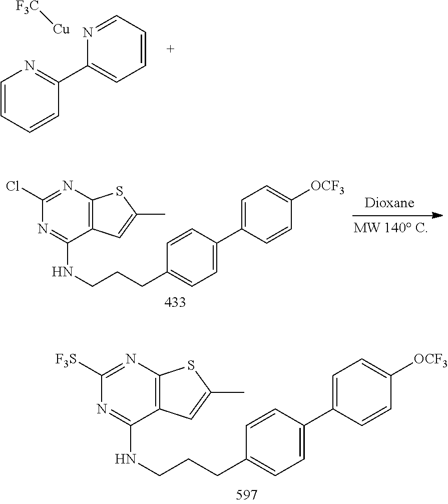

Example 433 (31.1 mg, 0.065 mmol) was dissolved in anhydrous dioxane (1.0 mL) and the reaction mixture was transferred to a 5 mL microwave vial. (bpy)Cu(SCF₃) (25.1 mg, 0.078 mmol) was added and nitrogen was bubbled through the reaction mixture for 1 minute before sealing and heating on a microwave reactor at 140° C. for 1.5 hours. The reaction was concentrated in vacuo and purified by prep HPLC to give 597: 3% yield; IUPAC: 6-methyl-N-(3-(4′-(trifluoromethoxy)-[1,1′-biphenyl]-4-yl)propyl)-2-((trifluoromethyl)thio)thieno[2,3-d]pyrimidin-4-amine; ¹H NMR (399 MHz, ACETONITRILE-d3) δ 7.69 (d, J=8.95 Hz, 2H), 7.49-7.59 (m, J=8.17 Hz, 2H), 7.34-7.41 (m, J=8.17 Hz, 2H), 7.31 (d, J=7.79 Hz, 2H), 6.91 (s, 1H), 6.46 (s, 1H), 3.51-3.58 (m, 2H), 2.72-2.77 (m, 2H), 2.50 (s, 3H), 1.75-1.80 (m, 2H); LCMS: ESI [M+H]⁺=544.1.

### Experimental Procedure for the Preparation of Example 598

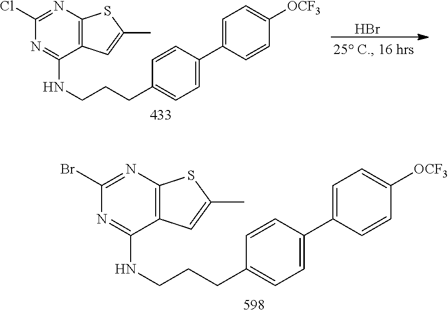 [SMILES: Cc1cc2c(NCCCc3ccc(-c4ccc(OC(F)(F)F)cc4)cc3)nc(Cl)nc2s1 ; Br ; Cc1cc2c(NCCCc3ccc(-c4ccc(OC(F)(F)F)cc4)cc3)nc(Br)nc2s1]

Example 433 (20.0 mg, 0.042 mmol) was dissolved in HBr in acetic acid (1.5 mL) and allowed to react for 16 hours at room temperature. The reaction was concentrated in vacuo and product was extracted into DCM (3×) from saturated NaHCO₃(aq). The organic layer was dried over Na₂SO₄, concentrated in vacuo and purified by prep HPLC to obtain 598: 89% yield; IUPAC: 2-bromo-6-methyl-N-(3-(4′-(trifluoromethoxy)-[1,1′-biphenyl]-4-yl)propyl)thieno[2,3-d]pyrimidin-4-amine; ¹H NMR (399 MHz, METHANOL-d4) δ 7.64 (d, J=8.56 Hz, 2H), 7.49 (d, J=7.79 Hz, 2H), 7.30 (d, J=7.40 Hz, 4H), 6.98 (s, 1H), 3.57 (t, J=7.01 Hz, 2H), 2.76 (t, J=7.40 Hz, 2H), 2.48 (s, 3H), 2.03 (quin, J=7.10 Hz, 2H); LCMS: ESI [M+H]⁺=522.1, 524.0.

### Experimental Procedure for the Preparation of Example 599

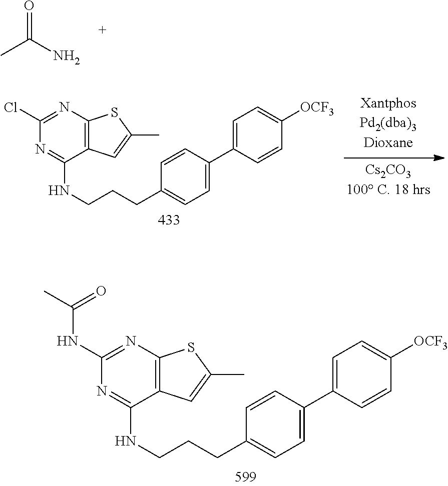

Example 433 (20.0 mg, 0.042 mmol) was added to a pressure vessel along with acetamide (2.5 mg. 0.042 mmol), Xantphos (3.6 mg, 0.006 mmol), Pd₂(dba)₃ (1.9 mg, 0.002 mmol) and Cs₂CO₃ (16.0 mg, 0.049 mmol). Anhydrous dioxane (2.0 mL) was added and nitrogen was bubbled through the reaction mixture for 2 minutes before sealing the pressure vessel. The reaction was heated at 100° C. for 18 hours before being concentrated in vacuo and purified by prep HPLC, followed by silica gel column chromatography to obtain 599: 6% yield; IUPAC: N-(6-methyl-4-((3-(4′-(trifluoromethoxy)-[1,1′-biphenyl]-4-yl)propyl)amino)thieno[2,3-d]pyrimidin-2-yl)acetamide; ¹H NMR (399 MHz, METHANOL-d4) δ 7.67 (d, J=8.56 Hz, 2H), 7.52 (d, J=8.18 Hz, 2H), 7.31 (t, J=8.56 Hz, 4H), 6.96 (s, 1H), 3.60 (t, J=6.81 Hz, 2H), 2.77 (t, J=7.40 Hz, 2H), 2.48 (s, 3H), 2.24 (br. s., 3H), 2.04 (dd, J=7.59, 14.21 Hz, 2H); LCMS: ESI [M+H]⁺=501.2.

### Experimental Procedure for the Preparation of Example 600

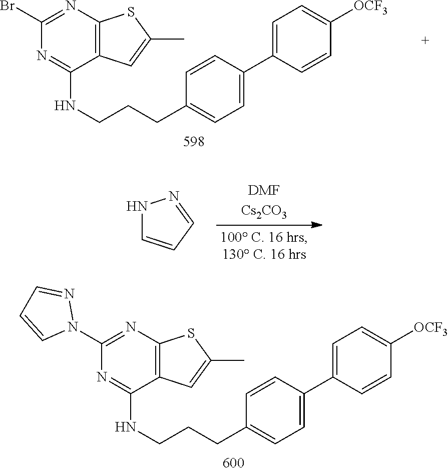 [SMILES: Cc1cc2c(NCCCc3ccc(-c4ccc(OC(F)(F)F)cc4)cc3)nc(Br)nc2s1 ; c1cn[nH]c1 ; Cc1cc2c(NCCCc3ccc(-c4ccc(OC(F)(F)F)cc4)cc3)nc(-n3cccn3)nc2s1]

Example 598 (16.7 mg, 0.032 mmol) was added to a reaction vial along with 1H-pyrazole (4.4 mg, 0.064 mmol) and Cs₂CO₃ (21.0 mg, 0.064 mmol). Anhydrous DMF (2.0 mL) was added and the reaction was heated at 100° C. for 18 hours. The temperature was increased to 130° C. for 18 hours after which time the reaction was cooled and passed through a 4 μM filter prior to purifying on prep HPLC to give 600: 10% yield; IUPAC: 6-methyl-2-(1H-pyrazol-1-yl)-N-(3-(4′-(trifluoromethoxy)-[1,1′-biphenyl]-4-yl)propyl)thieno[2,3-d]pyrimidin-4-amine; ¹H NMR (399 MHz, METHANOL-d4) δ 8.40 (s, 1H), 7.73 (s, 1H), 7.67 (d, J=8.56 Hz, 2H), 7.53 (d, J=8.17 Hz, 2H), 7.32 (d, J=7.79 Hz, 4H), 7.04 (s, 1H), 6.39 (s, 1H), 3.72 (t, J=7.01 Hz, 2H), 2.81 (t, J=7.40 Hz, 2H), 2.52 (s, 3H), 2.10 (quin, J=7.20 Hz, 2H); LCMS: ESI [M+H]⁺=510.1.

### Experimental Procedure for the Preparation of Example 601

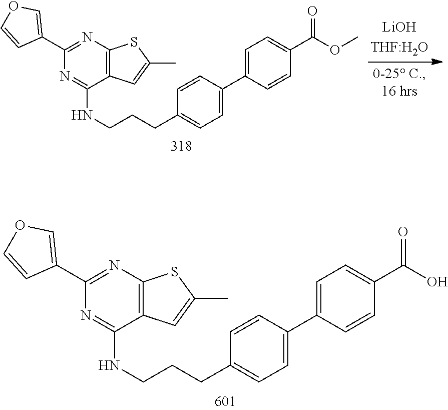 [SMILES: COC(=O)c1ccc(-c2ccc(CCCNc3nc(-c4ccoc4)nc4sc(C)cc34)cc2)cc1 ; Cc1cc2c(NCCCc3ccc(-c4ccc(C(=O)O)cc4)cc3)nc(-c3ccoc3)nc2s1]

Example 318 (10.0 mg, 0.021 mmol) was dissolved in THF (2.0 mL) and DI water (1.0 mL). The reaction was cooled on an ice bath before the addition of LiOH monohydrate (2.2 mg, 0.053 mmol), then allowed to slowly warm up to room temperature and react 16 hours before being concentrated in vacuo and purified by prep HPLC to give 601: 22% yield; IUPAC: 4′-(3-((2-(furan-3-yl)-6-methylthieno[2,3-d]pyrimidin-4-yl)amino)propyl)-[1,1′-biphenyl]-4-carboxylic acid; ¹H NMR (399 MHz, METHANOL-d4) δ 8.48 (s, 1H), 8.07 (d, J=5.06 Hz, 2H), 8.04 (s, 1H), 7.67 (d, J=8.17 Hz, 2H), 7.55-7.61 (m, 2H), 7.50 (s, 1H), 7.30-7.37 (m, J=8.17 Hz, 2H), 7.01 (s, 1H), 6.96 (s, 1H), 3.69 (t, J=7.20 Hz, 2H), 2.81 (t, J=7.20 Hz, 2H), 2.52 (s, 3H), 2.09 (td, J=7.20, 14.40 Hz, 2H); LCMS: ESI [M+H]⁺=470.1.

### Experimental Procedure for the Preparation of Example 602

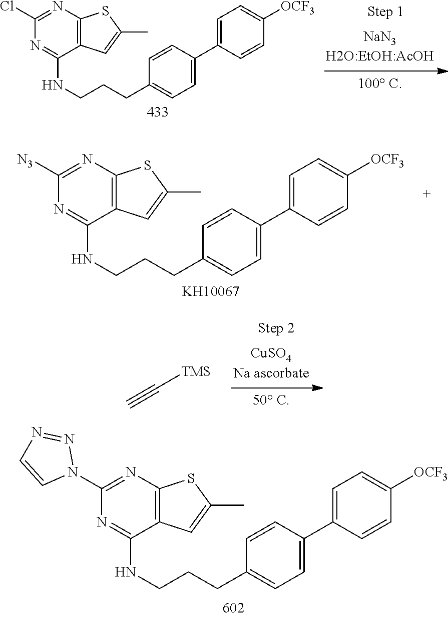

Step 1: Example 433 (10.0 mg, 0.021 mmol) was added to a pressure vessel along with NaN₃ (20.0 mg, 0.308 mmol). EtOH (2.0 mL), DI water (1.0 mL), and acetic acid (0.4 mL) were added sequentially to the reaction and the pressure vessel was sealed. The reaction was heated on an oil bath at 100° C. for 18 hours, after which more acetic acid (0.4 mL) and NaN₃ (20.0 mg, 0.308 mmol) were added, and the temperature was increased to 110° C. After 16 hours, the reaction was cooled to room temperature, saturated NaHCO₃(aq) (250 mL) was added, and the reaction was stirred for 10 minutes before filtering to collect precipitate. The solid was dried under high vacuum to give intermediate KH10067: 56% yield; IUPAC: 2-azido-6-methyl-N-(3-(4′-(trifluoromethoxy)-[1,1′-biphenyl]-4-yl)propyl)thieno[2,3-d]pyrimidin-4-amine; LCMS: ESI [M+H]⁺=485.1.

Step 2: Following the General Click Protocol, intermediate KH10067 (23.6 mg, 0.049 mmol) was reacted with (trimethylsilyl)acetylene (29.8 mg, 0.294 mmol) at 50° C. for 16 hours. Upon completion, the reaction mixture was concentrated in vacuo and purified by prep HPLC to give 602: 25% yield; IUPAC: 6-methyl-2-(1H-1,2,3-triazol-1-yl)-N-(3-(4′-(trifluoromethoxy)-[1,1′-biphenyl]-4-yl)propyl)thieno[2,3-d]pyrimidin-4-amine; ¹H NMR (399 MHz, METHANOL-d4) δ 8.54 (s, 1H), 7.76 (s, 1H), 7.66 (d, J=8.17 Hz, 2H), 7.51 (d, J=7.79 Hz, 2H), 7.32 (t, J=6.42 Hz, 4H), 7.09 (s, 1H), 3.72 (t, J=7.01 Hz, 2H), 2.81 (t, J=7.01 Hz, 2H), 2.54 (s, 3H), 2.10 (quin, J=7.10 Hz, 2H); LCMS: ESI [M+H]⁺=511.1.

### Experimental Procedure for the Preparation of Example 603

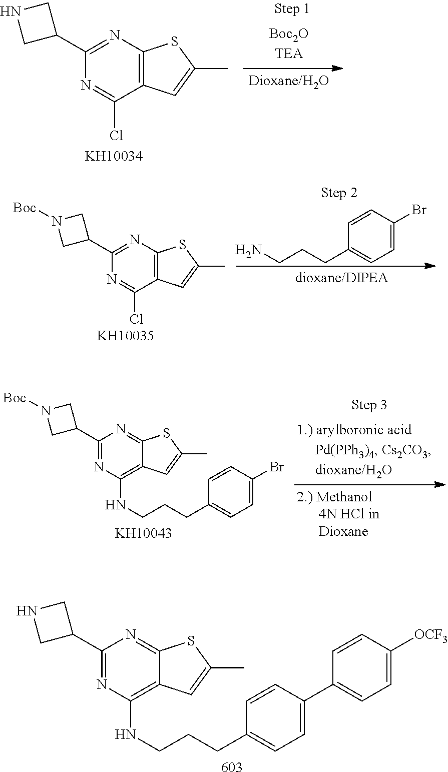

Step 1: Building block KH10034 (90.0 mg, 0.375 mmol) was dissolved in dioxane (2.0 mL) and DI water (0.5 mL). Triethylamine (104 μL, 0.750 mmol) was added and allowed to stir for a few minutes before the addition of Di-tert-butyl dicarbonate (98.0 mg, 0.450 mmol). Reaction was ran for 2 days at room temperature. Upon completion, the reaction mixture was concentrated in vacuo and purified by silica gel column chromatography to obtain the intermediate KH10035: 7% yield; IUPAC: tert-butyl 3-(4-chloro-6-methylthieno[2,3-d]pyrimidin-2-yl)azetidine-1-carboxylate; LCMS: ESI [M+Na]+=362.1.

Step 2: Following the protocol in Scheme 1, Method A, intermediate KH10035 (8.5 mg, 0.025 mmol) was reacted with 4-bromo-benzenepropanamine (6.4 mg, 0.030 mmol) for 16 hours at 90° C. The reaction was concentrated in vacuo and purified by prep HPLC to obtain intermediate KH10043: 40% yield; IUPAC: tert-butyl 3-(4-((3-(4-bromophenyl)propyl)amino)-6-methylthieno[2,3-d]pyrimidin-2-yl)azetidine-1-carboxylate; LCMS: ESI [M+H]⁺=517.1, 519.1.

Step 3: Following the General Suzuki Protocol, intermediate KH10043 (5.2 mg, 0.010 mmol) was reacted with 4-trifluoromethoxyphenylboronic acid (3.1 mg, 0.015 mmol), Pd(PPh₃)₄ (1.7 mg, 0.0015 mmol), and Cs₂CO₃ (4.9 mg, 0.105 mmol) at 90° C. for 2 hours. Upon completion, the reaction mixture was concentrated in vacuo and purified by prep HPLC. The purified boc-protected intermediate was dissolved in MeOH (1.7 mL) before the addition of [4N] HCl/dioxane (6.3 mL), and the reaction was stirred for 1 hour at room temperature. The reaction was concentrated in vacuo, and the product was purified via silica gel column chromatography using DCM and MeOH with 4% ammonia, to obtain 603: 17% yield; IUPAC: 2-(azetidin-3-yl)-6-methyl-N-(3-(4′-(trifluoromethoxy)-[1,1′-biphenyl]-4-yl)propyl)thieno[2,3-d]pyrimidin-4-amine; ¹H NMR (399 MHz, METHANOL-d4) δ 7.67 (d, J=8.17 Hz, 2H), 7.52 (d, J=7.79 Hz, 2H), 7.28-7.37 (m, 4H), 7.06 (s, 1H), 4.29-4.47 (m, 4H), 4.14 (d, J=9.34 Hz, 1H), 3.70 (t, J=6.42 Hz, 2H), 2.80 (t, J=7.01 Hz, 2H), 2.53 (s, 3H), 2.02-2.14 (m, 2H); LCMS: ESI [M+H]⁺=499.2.

### Experimental Procedure for the Preparation of Example 604

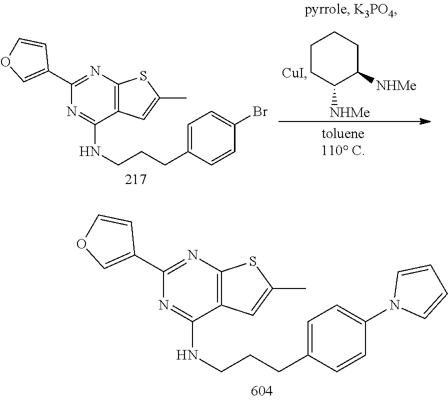 [SMILES: Cc1cc2c(NCCCc3ccc(Br)cc3)nc(-c3ccoc3)nc2s1 ; CN[C@@H]1CCCC[C@H]1NC ; Cc1cc2c(NCCCc3ccc(-n4cccc4)cc3)nc(-c3ccoc3)nc2s1]

Prior to use, N₂ gas was bubbled through anhydrous toluene in a flame dried flask for 3 h. Separately, example 217 (60.0 mg, 0.14 mmol), K₃PO₄ (52.0 mg, 0.26 mmol) and CuI (1.11 mg, 0.0058 mmol), were added to a flame dried flask with a stir bar, and the reaction was sealed and purged/flushed with N₂ (3×). Pyrrole (8.07 μL, 0.12 mmol), (1R,2R)—N,N-dimethylcyclohexane-1,2-diamine (3.7 μL, 0.023 mmol) and degassed toluene (0.25 mL) were added, and the reaction was heated for 24 hours at 110° C. After 24 hours, the incomplete reaction was cooled to room temperature and concentrated in vacuo. The crude reaction mixture was purified by silica gel column chromatography followed by prep HPLC to give 604: 11% yield; IUPAC: 2-(furan-3-yl)-6-methyl-N-(3-[4-(1H-pyrrol-1-yl)phenyl]propyl)thieno[2,3-d]pyrimidin-4-amine; ¹H NMR (399 MHz, ACETONITRILE-d3) δ 8.11 (s, 1H), 7.50 (d, J=0.78 Hz, 1H), 7.35-7.40 (m, 2H), 7.29-7.34 (m, 2H), 7.14 (s, 2H), 6.94 (d, J=5.45 Hz, 2H), 6.28 (s, 2H), 6.18 (br. s., 1H), 3.65 (q, J=6.49 Hz, 2H), 2.76 (t, J=7.40 Hz, 2H), 2.52 (s, 3H), 1.99-2.05 (m, 2H); LCMS: ESI [M+H]⁺=415.1.

### Experimental Procedure for the Preparation of Example 605

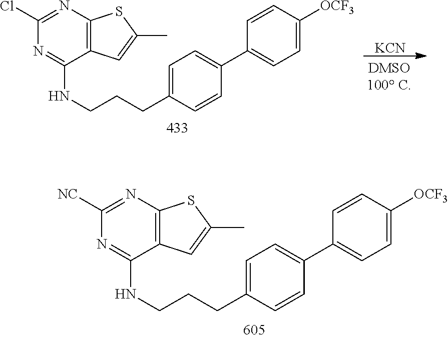 [SMILES: Cc1cc2c(NCCCc3ccc(-c4ccc(OC(F)(F)F)cc4)cc3)nc(Cl)nc2s1 ; "KCN" ; Cc1cc2c(NCCCc3ccc(-c4ccc(OC(F)(F)F)cc4)cc3)nc(C#N)nc2s1]

Example 433 (32.0 mg, 0.067 mmol) was dissolved in DMSO (1 mL). KCN (21.8 mg, 0.33 mmol) was added, and the vial was reacted at 100° C. for 1 day, then at 110° C. for 1 day. Upon completion, the reaction was cooled to room temperature, then the reaction mixture was passed through a 4 μM filter, and loaded directly onto prep HPLC for purification to give 605: 9% yield; IUPAC: 6-methyl-4-((3-[4′-(trifluoromethoxy)-[1,1′-biphenyl]-4-yl]propyl)amino)thieno[2,3-d]pyrimidine-2-carbonitrile; ¹H NMR (399 MHz, ACETONITRILE-d3) δ 7.70 (d, J=8.56 Hz, 2H), 7.55 (d, J=7.79 Hz, 2H), 7.29-7.41 (m, 4H), 7.02 (s, 1H), 6.57 (br. s., 1H), 3.58 (q, J=6.36 Hz, 2H), 2.78 (t, J=7.40 Hz, 2H), 2.56 (s, 3H), 2.01-2.06 (m, 2H); LCMS: ESI [M+H]⁺=469.1.

### Example 14: Biological Activity

### MABA Assay

The compounds were evaluated in the Microplate Alamar Blue Assay (MABA), which is commonly used to evaluate the efficacy of compounds in restraining Mtb growth (Franzblau et al., “Comprehensive analysis of methods used for the evaluation of compounds against Mycobacterium tuberculosis,” Tuberculosis (Edinb.), 92:453-488, 2012). The MABA utilizes the dye resazurin, which is dark blue and nonfluorescent in its oxidized form but becomes pink and fluorescent when reduced to resorufin as a result of cellular metabolism. The degree of this color change is monitored and quantified, and compounds that inhibit Mtb growth or survival will decrease or block this color change. By performing this assay with WT Mtb Erdman strain in Middlebrook 7H9 media (Sigma-Aldrich) with a range of concentrations of each compound, the MIC₅₀ and MIC₉₀ values for active compounds were able to be calculated, which is defined as the concentration of antibiotic that inhibits mycobacterial survival by 50% and 90%, respectively. These assays were performed in 96 well dishes and the compounds were added to the cultures at the time of Mtb inoculation and incubated for 7 days at 37° C. in 5% CO₂, at which point the resazurin dye was added for 24 hours before measuring the fluorescence at excitation 530 nm and emission 590 nm.

The present compounds have an activity as shown in Table 13 below. N.D. indicated “not determined.”

TABLE 13
Activity of Compounds Against Mtb

| | Avg. | | MABA | | Avg. |
|---|---|---|---|---|---|
| | IC50 | | Avg. IC50 | | IC50 |
| Cmpd. No. | (μM) | Cmpd. No. | (μM) | Cmpd. No. | (μM) |
| 328 | <0.001 | 4 | 0.300 | 146 | 5.100 |
| 314 | 0.003 | 443 | 0.300 | 444 | 5.500 |
| 567 | <0.004 | 288 | 0.310 | 129 | 5.600 |
| 535 | 0.004 | 54 | 0.310 | 99 | 5.700 |
| 558 | <0.004 | 470 | 0.330 | 583 | 5.800 |
| 557 | <0.004 | 271 | 0.350 | 229 | 5.900 |
| 394 | <0.004 | 71 | 0.350 | 91 | 5.900 |
| 556 | <0.004 | 207 | 0.350 | 162 | 6.000 |
| 387 | 0.005 | 170 | 0.360 | 66 | 6.000 |
| 396 | <0.005 | 191 | 0.360 | 44 | 6.300 |
| 330 | <0.005 | 97 | 0.360 | 597 | 6.300 |
| 395 | 0.005 | 599 | >0.370 | 218 | 6.400 |
| 384 | 0.006 | 310 | 0.380 | 107 | >6.400 |
| 502 | 0.006 | 150 | 0.380 | 132 | 6.600 |
| 322 | 0.006 | 312 | 0.380 | 239 | >6.800 |
| 388 | 0.007 | 290 | 0.390 | 85 | >6.800 |
| 289 | 0.008 | 225 | 0.390 | 158 | >6.900 |
| 402 | 0.008 | 295 | 0.400 | 514 | 7.000 |
| 382 | 0.008 | 59 | 0.430 | 31 | 7.100 |
| 377 | 0.008 | 6 | 0.440 | 220 | >7.200 |
| 334 | 0.008 | 134 | 0.440 | 145 | 7.300 |
| 291 | 0.008 | 152 | 0.440 | 17 | 7.600 |
| 434 | 0.010 | 196 | 0.450 | 219 | >7.700 |
| 545 | 0.010 | 10 | 0.450 | 84 | 7.700 |
| 325 | 0.010 | 398 | >0.460 | 117 | >7.700 |
| 559 | 0.011 | 262 | 0.470 | 108 | >7.800 |
| 531 | 0.012 | 80 | 0.470 | 37 | >7.900 |
| 379 | 0.012 | 79 | 0.480 | 243 | >8.000 |
| 333 | 0.012 | 167 | 0.490 | 42 | 8.100 |
| 315 | 0.012 | 101 | 0.490 | 440 | 8.100 |
| 263 | 0.013 | 106 | 0.490 | 197 | 8.200 |
| 432 | 0.014 | 450 | 0.490 | 210 | 8.200 |
| 458 | 0.014 | 400 | >0.500 | 23 | 8.300 |
| 546 | 0.015 | 166 | <0.510 | 39 | >8.800 |
| 326 | 0.015 | 270 | 0.520 | 47 | >8.800 |
| 336 | 0.015 | 574 | >0.520 | 16 | 9.000 |
| 338 | 0.015 | 590 | 0.530 | 187 | 9.100 |
| 339 | <0.015 | 593 | 0.530 | 505 | 9.100 |
| 343 | <0.015 | 601 | 0.530 | 490 | >9.100 |
| 427 | 0.016 | 190 | 0.550 | 390 | 9.300 |
| 393 | 0.016 | 259 | 0.560 | 171 | >9.500 |
| 292 | 0.016 | 119 | 0.560 | 235 | >9.900 |
| 55 | 0.017 | 255 | 0.570 | 501 | >10.000 |
| 425 | 0.017 | 511 | 0.570 | 157 | >10.000 |
| 380 | 0.017 | 252 | 0.600 | 159 | >10.000 |
| 528 | 0.018 | 201 | 0.610 | 163 | >10.000 |
| 430 | 0.018 | 512 | 0.610 | 169 | >10.000 |
| 428 | 0.020 | 125 | 0.610 | 180 | >10.000 |
| 429 | 0.020 | 114 | 0.620 | 182 | >10.000 |
| 431 | 0.020 | 277 | 0.630 | 183 | >10.000 |
| 318 | 0.020 | 115 | 0.640 | 238 | >10.000 |
| 369 | 0.021 | 200 | 0.650 | 184 | >10.000 |
| 103 | 0.022 | 128 | 0.660 | 186 | >10.000 |
| 370 | 0.025 | 195 | 0.680 | 242 | >10.000 |
| 340 | 0.025 | 136 | 0.690 | 246 | >10.000 |
| 298 | 0.026 | 8 | 0.690 | 589 | >10.000 |
| 299 | 0.026 | 468 | 0.690 | 194 | >10.000 |
| 302 | 0.027 | 278 | 0.710 | 562 | >10.000 |
| 311 | 0.027 | 22 | 0.770 | 591 | >10.000 |
| 102 | 0.027 | 113 | 0.800 | 285 | >10.000 |
| 604 | 0.028 | 52 | 0.810 | 12 | >10.000 |
| 596 | 0.029 | 62 | 0.810 | 21 | >10.000 |
| 555 | 0.029 | 307 | 0.830 | 221 | >10.000 |
| 549 | 0.029 | 375 | 0.860 | 41 | >10.000 |
| 435 | 0.030 | 519 | 0.900 | 51 | >10.000 |
| 374 | 0.030 | 192 | 0.930 | 67 | >10.000 |
| 331 | 0.030 | 276 | 0.930 | 68 | >10.000 |
| 561 | 0.031 | 94 | 0.930 | 73 | >10.000 |
| 337 | 0.031 | 533 | 0.930 | 74 | >10.000 |
| 389 | 0.032 | 244 | 1.000 | 89 | >10.000 |
| 547 | 0.033 | 373 | 1.000 | 507 | >10.000 |
| 297 | 0.034 | 24 | 1.000 | 508 | >10.000 |
| 329 | 0.034 | 449 | >1.000 | 95 | >10.000 |
| 503 | 0.035 | 454 | >1.000 | 98 | >10.000 |
| 303 | 0.037 | 568 | >1.000 | 579 | >10.000 |
| 323 | 0.037 | 457 | >1.000 | 515 | >10.000 |
| 273 | 0.038 | 160 | 1.100 | 580 | >10.000 |
| 381 | 0.039 | 587 | 1.100 | 520 | >10.000 |
| 304 | 0.040 | 253 | 1.100 | 524 | >10.000 |
| 264 | 0.040 | 269 | 1.100 | 109 | >10.000 |
| 424 | 0.041 | 279 | 1.100 | 526 | >10.000 |
| 459 | 0.043 | 7 | 1.100 | 527 | >10.000 |
| 281 | 0.044 | 50 | 1.100 | 116 | >10.000 |
| 392 | 0.044 | 69 | 1.100 | 137 | >10.000 |
| 448 | 0.044 | 82 | 1.100 | 138 | >10.000 |
| 301 | 0.045 | 205 | 1.200 | 584 | >10.000 |
| 306 | 0.047 | 268 | 1.200 | 139 | >10.000 |
| 266 | 0.049 | 26 | 1.200 | 140 | >10.000 |
| 532 | 0.049 | 86 | >1.200 | 142 | >10.000 |
| 441 | 0.049 | 564 | 1.200 | 529 | >10.000 |
| 560 | 0.050 | 141 | 1.200 | 144 | >10.000 |
| 313 | 0.050 | 226 | 1.200 | 148 | >10.000 |
| 433 | 0.054 | 178 | 1.300 | 492 | >10.000 |
| 376 | 0.055 | 181 | 1.300 | 493 | >10.000 |
| 110 | 0.055 | 232 | 1.300 | 494 | >10.000 |
| 455 | 0.056 | 581 | 1.300 | 495 | >10.000 |
| 305 | 0.057 | 236 | 1.400 | 496 | >10.000 |
| 447 | 0.057 | 247 | 1.400 | 153 | >10.000 |
| 453 | 0.058 | 228 | 1.500 | 154 | >10.000 |
| 18 | 0.060 | 231 | 1.500 | 497 | >10.000 |
| 124 | 0.062 | 254 | 1.500 | 155 | >10.000 |
| 391 | 0.062 | 222 | 1.500 | 605 | >10.000 |
| 378 | 0.063 | 87 | 1.500 | 456 | >10.000 |
| 539 | 0.068 | 566 | 1.600 | 14 | >10.000 |
| 61 | 0.070 | 224 | 1.600 | 13 | >10.000 |
| 403 | 0.070 | 164 | 1.700 | 504 | >10.000 |
| 569 | 0.070 | 179 | 1.700 | 595 | N.D |
| 577 | 0.071 | 223 | 1.700 | 423 | N.D |
| 344 | 0.074 | 64 | 1.700 | 463 | N.D |
| 274 | 0.076 | 234 | 1.800 | 217 | N.D |
| 300 | 0.076 | 237 | 1.800 | 215 | N.D |
| 426 | 0.076 | 245 | 1.800 | 216 | N.D |
| 452 | 0.076 | 248 | 1.800 | 509 | N.D |
| 332 | 0.080 | 193 | 1.800 | 530 | N.D |
| 598 | 0.081 | 123 | 1.800 | 404 | N.D |
| 118 | 0.082 | 451 | 1.800 | 405 | N.D |
| 437 | 0.085 | 204 | 1.900 | 406 | N.D |
| 534 | 0.086 | 63 | 1.900 | 342 | N.D |
| 335 | 0.087 | 518 | 1.900 | 345 | N.D |
| 536 | 0.088 | 165 | 2.000 | 346 | N.D |
| 203 | 0.089 | 9 | 2.000 | 347 | N.D |
| 294 | 0.089 | 38 | 2.000 | 407 | N.D |
| 575 | 0.091 | 49 | 2.000 | 408 | N.D |
| 76 | 0.092 | 75 | 2.000 | 409 | N.D |
| 439 | 0.093 | 90 | 2.000 | 410 | N.D |
| 446 | 0.096 | 104 | 2.000 | 411 | N.D |
| 176 | 0.097 | 105 | 2.000 | 412 | N.D |
| 251 | 0.100 | 149 | 2.000 | 554 | N.D |
| 309 | 0.100 | 233 | 2.100 | 413 | N.D |
| 327 | 0.100 | 422 | 2.100 | 414 | N.D |
| 538 | 0.100 | 5 | 2.100 | 415 | N.D |
| 510 | 0.110 | 15 | 2.100 | 416 | N.D |
| 516 | 0.110 | 28 | 2.100 | 573 | N.D |
| 199 | 0.120 | 36 | 2.100 | 348 | N.D |
| 25 | 0.120 | 147 | 2.100 | 349 | N.D |
| 506 | 0.120 | 30 | 2.200 | 350 | N.D |
| 211 | 0.120 | 35 | 2.200 | 351 | N.D |
| 282 | 0.130 | 46 | 2.200 | 352 | N.D |
| 286 | 0.130 | 88 | 2.200 | 417 | N.D |
| 96 | 0.130 | 130 | 2.200 | 418 | N.D |
| 121 | 0.130 | 401 | 2.200 | 419 | N.D |
| 543 | 0.130 | 185 | 2.300 | 353 | N.D |
| 537 | 0.130 | 29 | 2.300 | 367 | N.D |
| 257 | 0.140 | 43 | 2.400 | 552 | N.D |
| 280 | 0.140 | 230 | 2.500 | 553 | N.D |
| 438 | 0.140 | 293 | 2.500 | 550 | N.D |
| 77 | 0.140 | 83 | 2.500 | 551 | N.D |
| 341 | 0.140 | 48 | 2.600 | 354 | N.D |
| 265 | 0.150 | 126 | 2.600 | 355 | N.D |
| 371 | 0.150 | 92 | 2.700 | 356 | N.D |
| 542 | 0.150 | 189 | 2.800 | 357 | N.D |
| 420 | 0.160 | 11 | 2.800 | 358 | N.D |
| 78 | 0.160 | 45 | 2.800 | 359 | N.D |
| 156 | 0.160 | 65 | 2.800 | 360 | N.D |
| 324 | 0.160 | 112 | 2.800 | 361 | N.D |
| 445 | 0.160 | 127 | 2.800 | 464 | N.D |
| 198 | 0.170 | 240 | 2.900 | 572 | N.D |
| 421 | 0.170 | 284 | 2.900 | 362 | N.D |
| 582 | 0.170 | 19 | 2.900 | 363 | N.D |
| 469 | 0.170 | 81 | 3.000 | 364 | N.D |
| 275 | 0.180 | 120 | 3.000 | 365 | N.D |
| 465 | 0.180 | 131 | 3.000 | 366 | N.D |
| 258 | 0.190 | 241 | 3.100 | 600 | N.D |
| 287 | 0.190 | 2 | 3.100 | 540 | N.D |
| 122 | 0.190 | 517 | 3.100 | 212 | N.D |
| 602 | 0.190 | 168 | 3.200 | 603 | N.D |
| 586 | 0.200 | 283 | 3.200 | 460 | N.D |
| 250 | 0.200 | 100 | 3.200 | 461 | N.D |
| 260 | 0.200 | 208 | 3.200 | 316 | N.D |
| 385 | 0.200 | 173 | 3.300 | 462 | N.D |
| 467 | 0.200 | 227 | 3.300 | 481 | N.D |
| 368 | 0.210 | 576 | 3.400 | 213 | N.D |
| 27 | 0.210 | 499 | 3.400 | 482 | N.D |
| 111 | 0.210 | 32 | 3.500 | 483 | N.D |
| 436 | 0.220 | 500 | 3.600 | 484 | N.D |
| 143 | 0.220 | 40 | 3.600 | 473 | N.D |
| 386 | 0.220 | 72 | 3.900 | 485 | N.D |
| 70 | 0.230 | 249 | 4.000 | 486 | N.D |
| 513 | 0.230 | 578 | 4.000 | 487 | N.D |
| 135 | 0.230 | 57 | 4.100 | 474 | N.D |
| 383 | 0.230 | 498 | 4.100 | 475 | N.D |
| 442 | 0.230 | 172 | 4.200 | 476 | N.D |
| 570 | 0.230 | 56 | 4.200 | 477 | N.D |
| 175 | <0.240 | 93 | 4.200 | 478 | N.D |
| 308 | 0.240 | 522 | 4.200 | 479 | N.D |
| 1 | 0.240 | 33 | 4.300 | 317 | N.D |
| 53 | 0.240 | 565 | 4.300 | 571 | N.D |
| 594 | 0.240 | 161 | 4.400 | 480 | N.D |
| 372 | 0.250 | 588 | 4.400 | 319 | N.D |
| 521 | 0.250 | 592 | 4.400 | 320 | N.D |
| 202 | 0.260 | 491 | 4.400 | 214 | N.D |
| 206 | 0.260 | 523 | 4.500 | 321 | N.D |
| 151 | 0.260 | 177 | 4.700 | 466 | N.D. |
| 272 | 0.270 | 563 | 4.700 | 472 | N.D. |
| 296 | 0.270 | 490 | 4.700 | 471 | N.D. |
| 34 | 0.270 | 525 | 4.800 | 488 | N.D. |
| 133 | 0.270 | 20 | 4.900 | 541 | N.D. |
| 399 | >0.270 | 60 | 4.900 | | |
| 261 | 0.280 | 209 | 4.900 | | |
| 3 | 0.280 | 58 | 5.000 | | |
| 256 | 0.290 | 397 | 5.000 | | |
| 267 | 0.300 | 188 | 5.100 | | |

### Example 15: Compound Profiles and Animal Efficacy

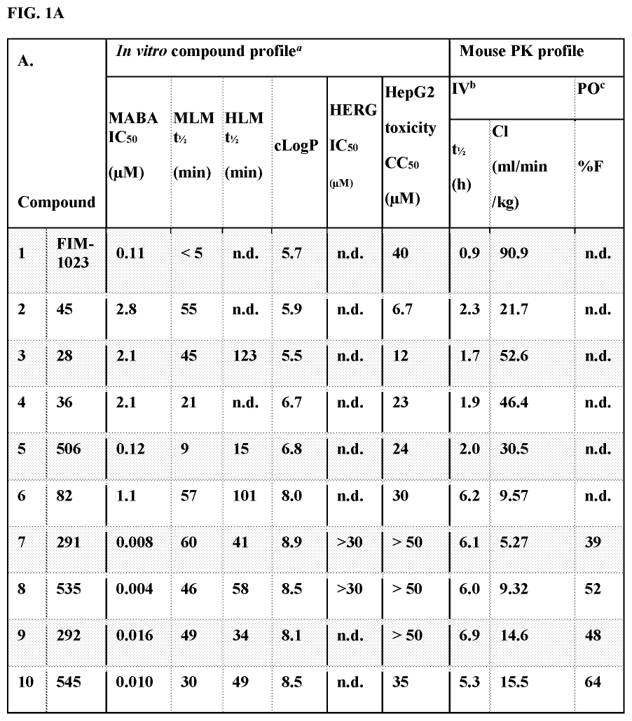

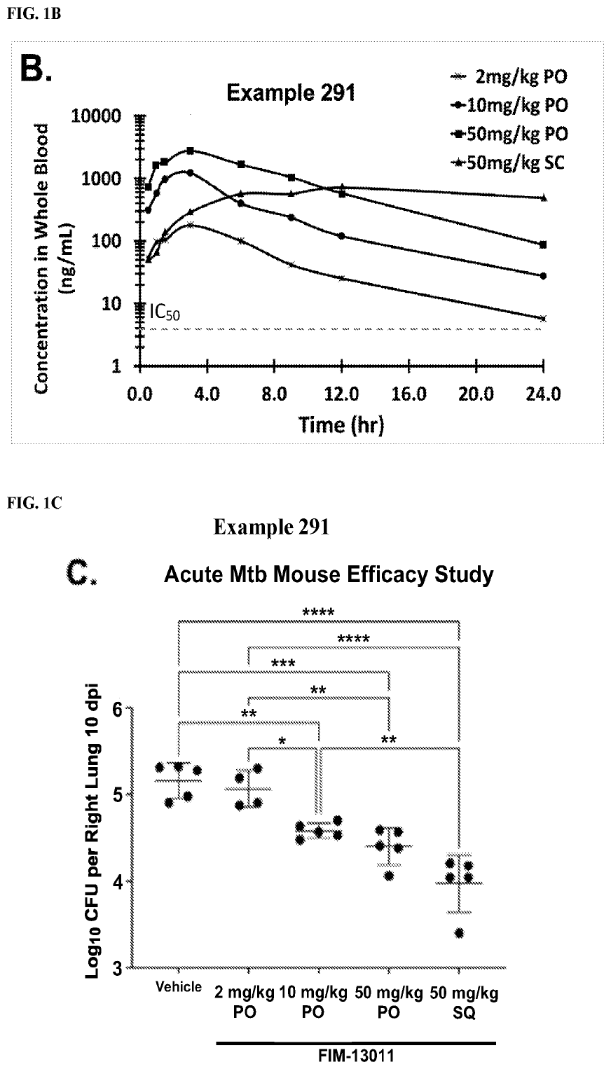

In vitro compound profiles were generated and in vivo pharmacokinetic (PK) studies were performed on 10 compounds and the results are shown in the table of FIG. 1, panel (A). FIM-1023 is 2-ethyl-6-methyl-N-(3-phenylpropyl)thieno[2,3-d]pyrimidin-4-amine:

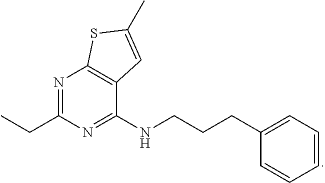 [SMILES: CCc1nc(NCCCc2ccccc2)c2cc(C)sc2n1]

The PK studies were performed by administering IV and oral doses of the compounds listed to mice. The IV bolus dose was 1 mg/kg and the oral dose was 2 mg/kg. The key for FIG. 1, panel (A) is as follows n.d.=not determined; ^{a}In vitro profile: MLM/HLM=mouse/human liver microsome, t_{1/2}=half-life, cLogP=measure of lipophilicity, hERG IC₅₀=concentration at which human ether-a-go-go-related gene activity is inhibited 50% (a measure of potential for cardiotoxicity), HepG2 liver cell toxicity CC₅₀=concentration at which cytotoxicity is 50%; ^{b}IV bolus dose (1 mg/kg): t_{1/2}=plasma half-life, Cl=clearance; ° Oral (PO) dose (2 mg/kg): % F=oral bioavailability.

Panel (B) of FIG. 1 presents the results of a dose escalation PK study for 291. Panel (C) of FIG. 1 presents the results of an efficacy study using 291 in a BALB/cJ mouse model of acute Mtb lung infection. Groups of mice are treated once daily with the same dose range of 291 used in panel (B) or vehicle alone for 9 days starting 1 day after a low dose aerosol infection (˜100 CFU seeded per lung) with the Mtb Erdman strain. The statistical analysis of log₁₀-transformed efficacy data was by one-way ANOVA with Tukey's correction for multiple comparisons where ** indicates P<0.05, **P<0.01, ***P<0.001, ****P<0.0001.

When introducing elements of the present disclosure, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compounds, compositions, methods, and processes without departing from the scope of the invention, it is intended that all matter contained in the above description shall be interpreted as illustrative and not in a limiting sense.

1. A compound of Formula I, or a pharmaceutically acceptable salt thereof: 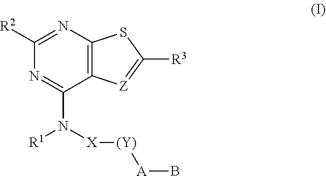
wherein:
R¹ is H, C₁-C₆ alkyl, —COOR′, —COR′, or —C(O)NHR′, where R′ is H, alkyl, cycloalkyl, or aryl;
R² is optionally substituted C₁-C₆ alkyl, optionally substituted C₃-C₆ cycloalkyl, optionally substituted C₂-C₆ alkenyl, optionally substituted aryl, optionally substituted aralkyl, optionally substituted heteroaryl, optionally substituted heteroalkyl, optionally substituted heterocycloalkyl, halo, haloalkyl, cyano, acyl, —C(CH₂)CH₃, —N₃, —CH₂OH, —CH₂OR⁴, —CH₂SR⁴, —OR⁴, —SR⁴, —SOR⁴, —SO₂R⁴, —CH₂NR⁵R⁶, —NR⁵R⁶, —CO₂H, —COR⁴, —CO₂R⁴, —NHCONR⁵R⁶, —C(R⁴)OH, —NHCOR⁴, or —CONR⁵R⁶;
R³ is H, C₁-C₆ alkyl, C₁-C₆ cycloalkyl, halo, or haloalkyl;
R⁴ is C₁-C₆ alkyl, C₃-C₆ cycloalkyl, haloalkyl, heteroalkyl, or aryl;
R⁵ and R⁶ are each independently H, optionally substituted C₁-C₆ alkyl, or optionally substituted C₃-C₆ cycloalkyl, or R⁵ and R⁶ form an optionally substituted C₃₋₆ cycloalkyl, optionally substituted heterocycloalkyl, optionally substituted heteroalkyl, optionally substituted aryl, or optionally substituted heteroaryl;
X is optionally substituted C₁-C₆ alkylene, optionally substituted C₃-C₆ cycloalkylene, optionally substituted C₂-C₆ alkenylene, optionally substituted C₃-C₆ cycloalkenylene, optionally substituted C₂-C₆ alkynylene, optionally substituted C₄-C₆ cycloalkynylene, optionally substituted carbonyl, —C(O)(CH)ₙ—, or —C(O)NH₂(CH)ₙ—, where n is an integer from 0 to 6;
Y is a bond, optionally substituted C₁-C₃ alkylene, optionally substituted amino, oxygen, sulfur, sulfoxide, or sulfone;
A is optionally substituted heterocycloalkyl, optionally substituted aryl, optionally substituted aralkyl, optionally substituted heteroaryl, or optionally substituted heteroaralkyl;
B is optionally substituted alkyl, optionally substituted cycloalkyl, optionally substituted heterocycloalkyl, haloalkyl, optionally substituted alkoxy, haloalkoxy, halo, cyano, optionally substituted aryl, optionally substituted aralkyl, optionally substituted heteroaryl, optionally substituted heteroaralkyl, optionally substituted arenediyl, —OCF₃, or —SF₅; and
Z is CH or N.

2.-20. (canceled)

21. The compound of claim 1, wherein B is:

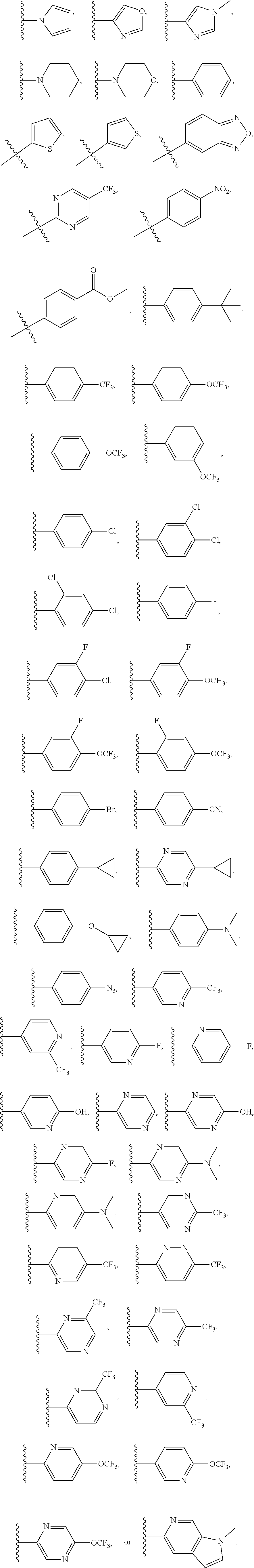

22.-29. (canceled)

30. A compound of Formula IA, or a pharmaceutically acceptable salt thereof: 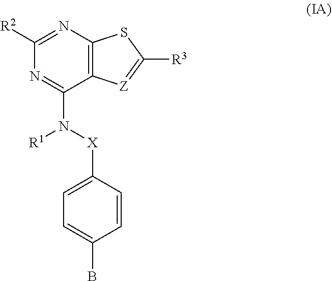
wherein:
R¹ is H or C₁-C₆ alkyl;
R² is optionally substituted C₂-C₆ alkenyl, optionally substituted heteroaryl, optionally substituted heteroalkyl, —OR⁴, —SR⁴, or —NR⁵R⁶;
R³ is H, C₁-C₆ alkyl, or haloalkyl;
R⁴ is C₁-C₆ alkyl;
R⁵ and R⁶ are each independently H or optionally substituted C₁-C₆ alkyl;
X is optionally substituted C₁-C₆ alkylene;
B is 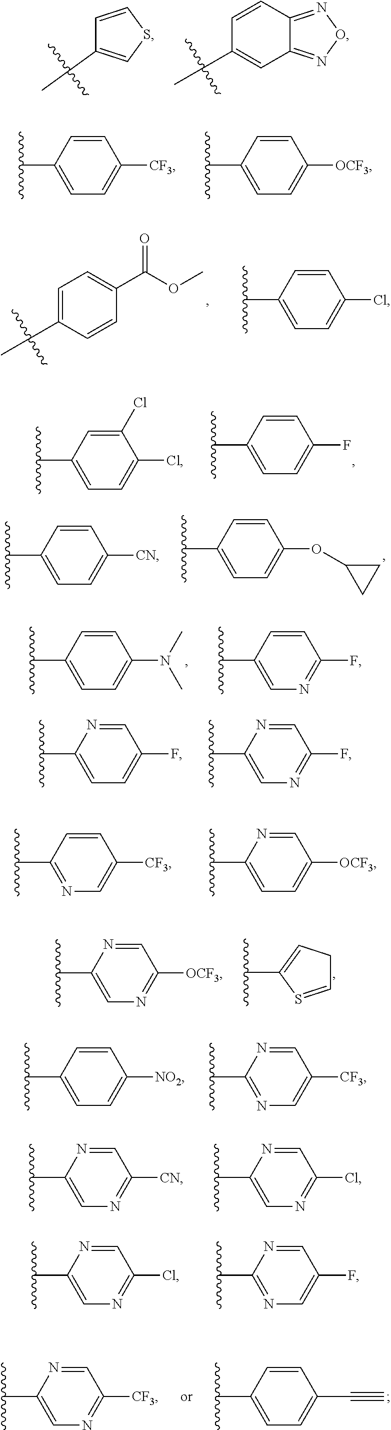
and
Z is CH or N.

31. The compound of claim 30, wherein R¹ is H.

32. The compound of claim 31, wherein R² is furyl, isoxazolyl, thiophenyl, pyrrolyl, —OR⁴, —SR⁴, or —NR⁵R⁶; wherein R⁴ is C₁-C₃ alkyl, and R⁵ and R⁶ are each independently C₁-C₃ alkyl.

33. The compound of claim 32, wherein R⁴ is independently methyl and R⁵ and R⁶ is methyl.

34. The compound of claim 33, wherein R³ is methyl.

35. The compound of claim 34, wherein X is ethylene, propylene, butylene, or pentylene.

36. The compound of claim 35, wherein X is propylene.

37. The compound of claim 30, wherein B is

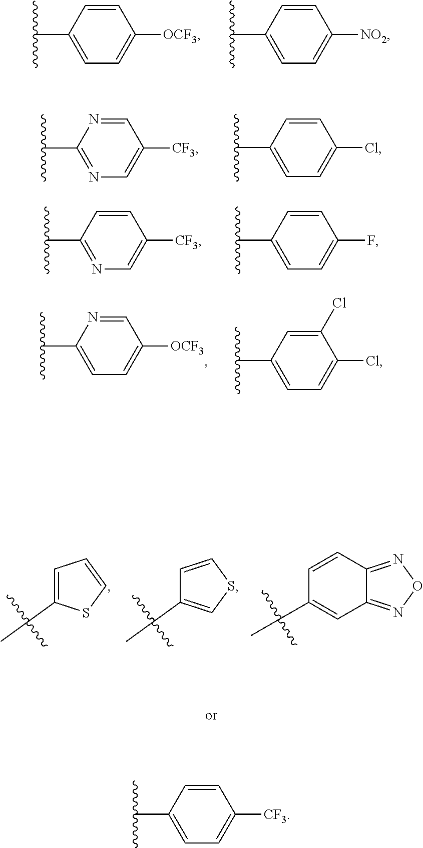

38. A compound selected from the group consisting of:

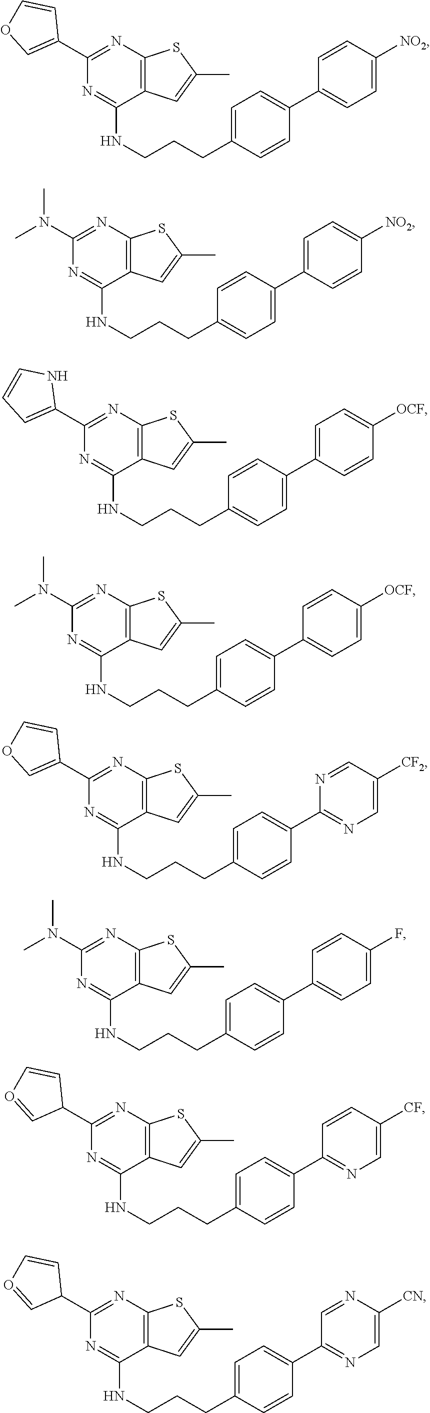

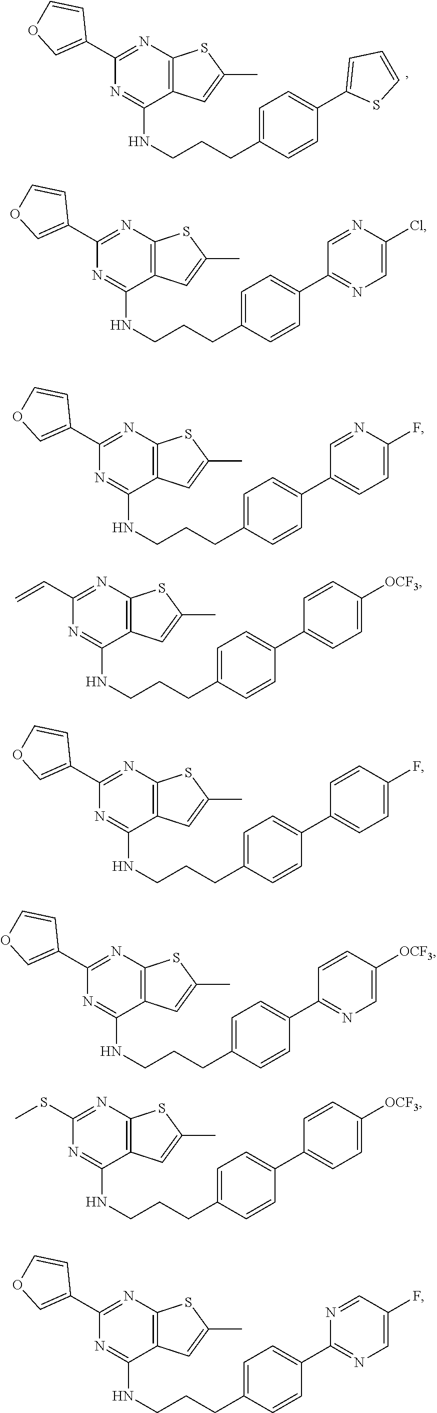

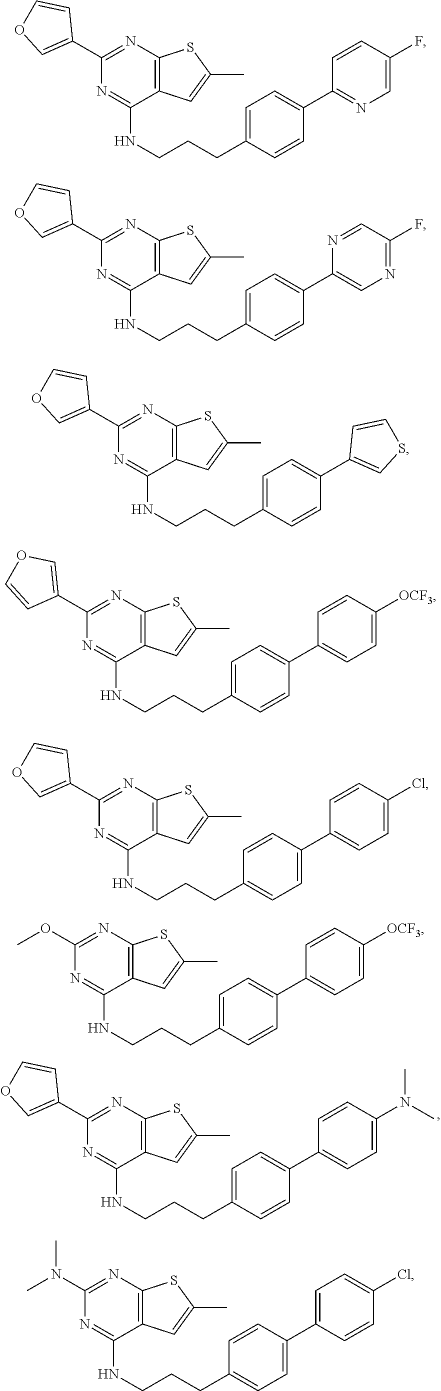

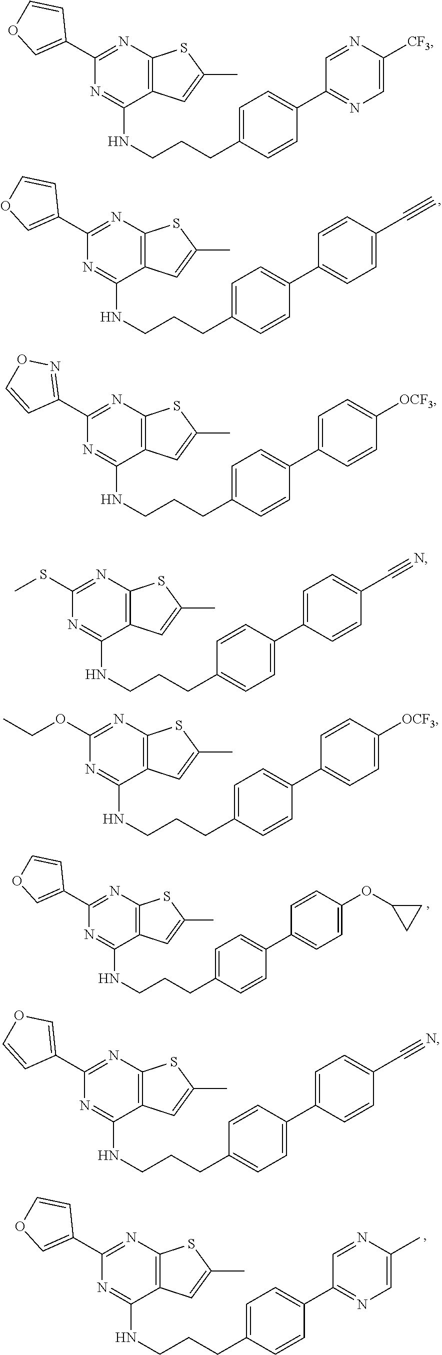

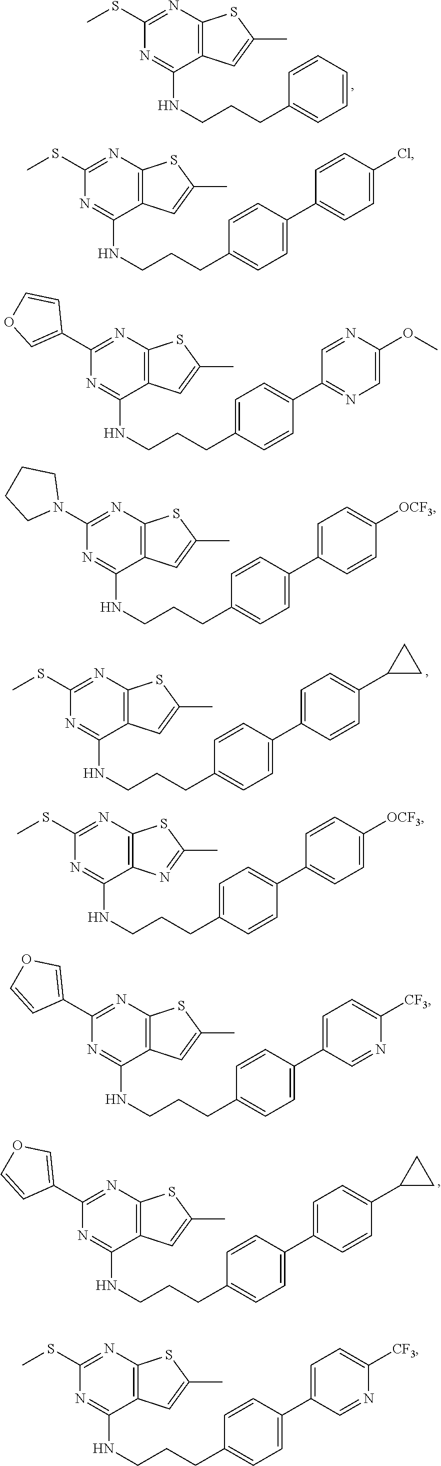

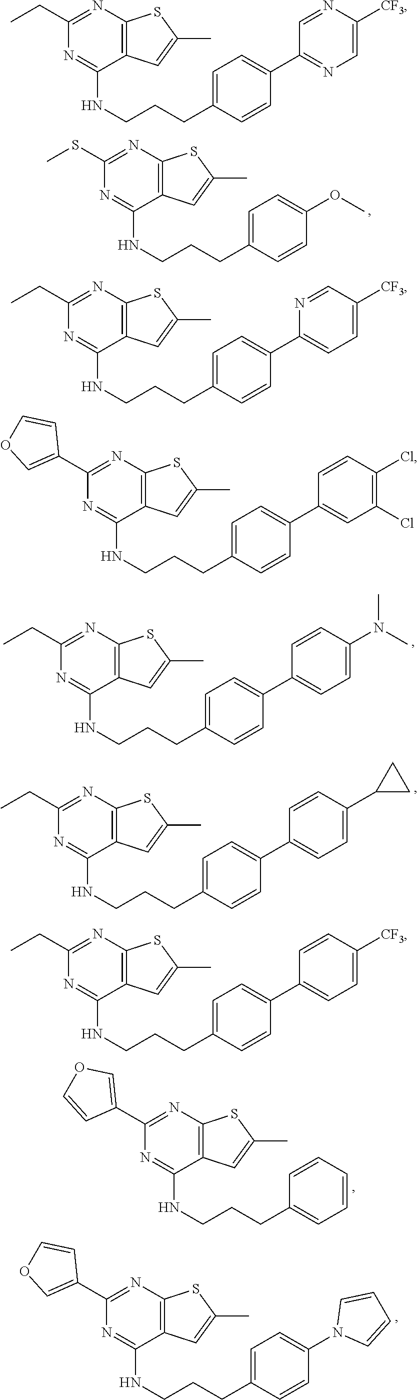

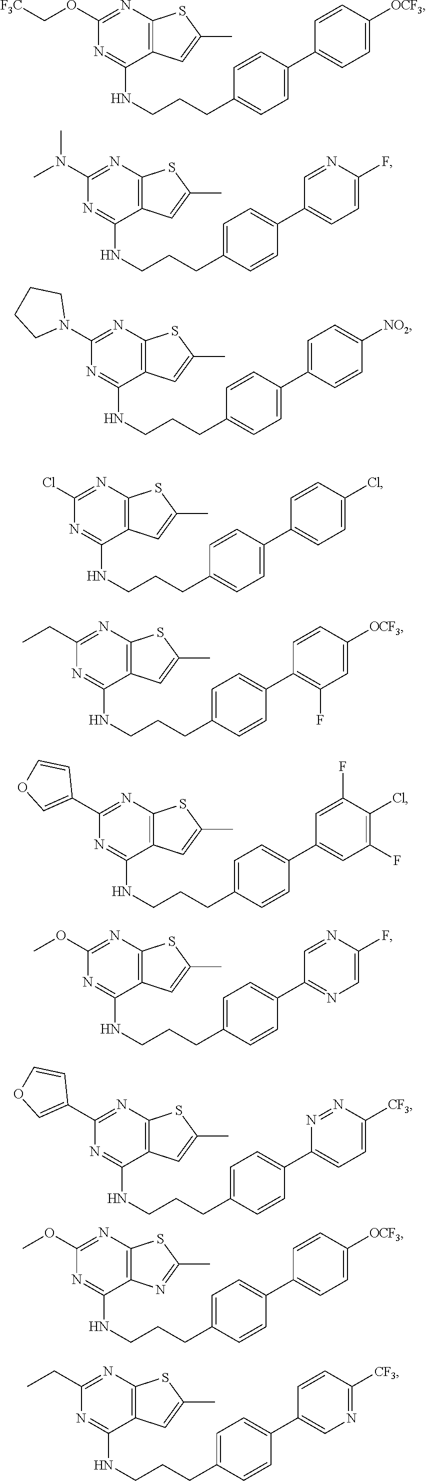

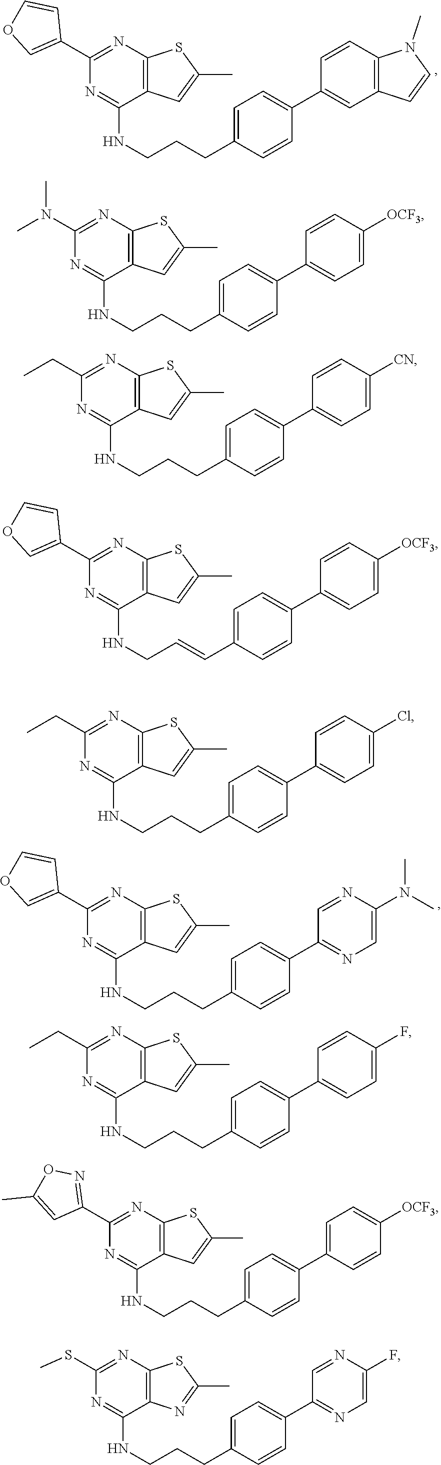

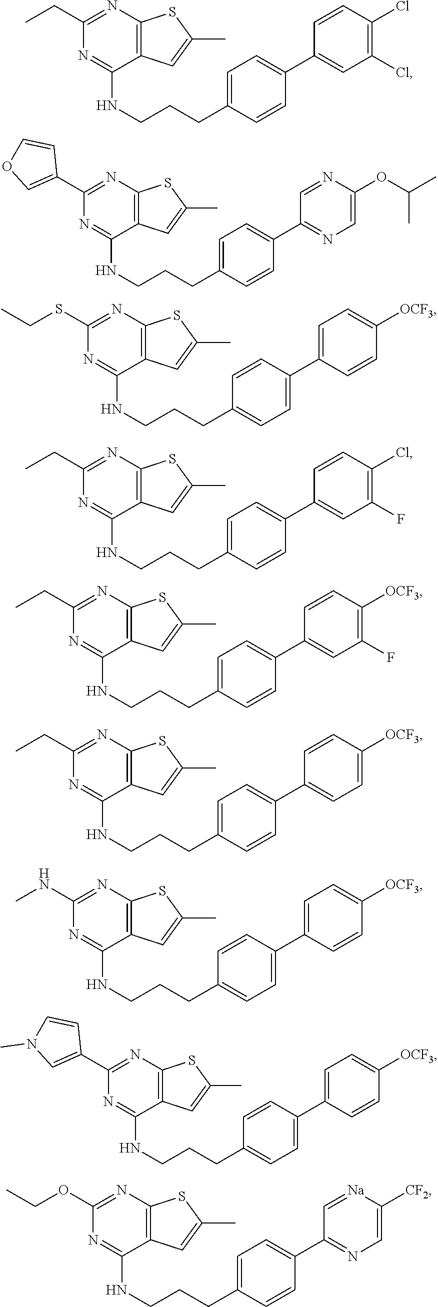

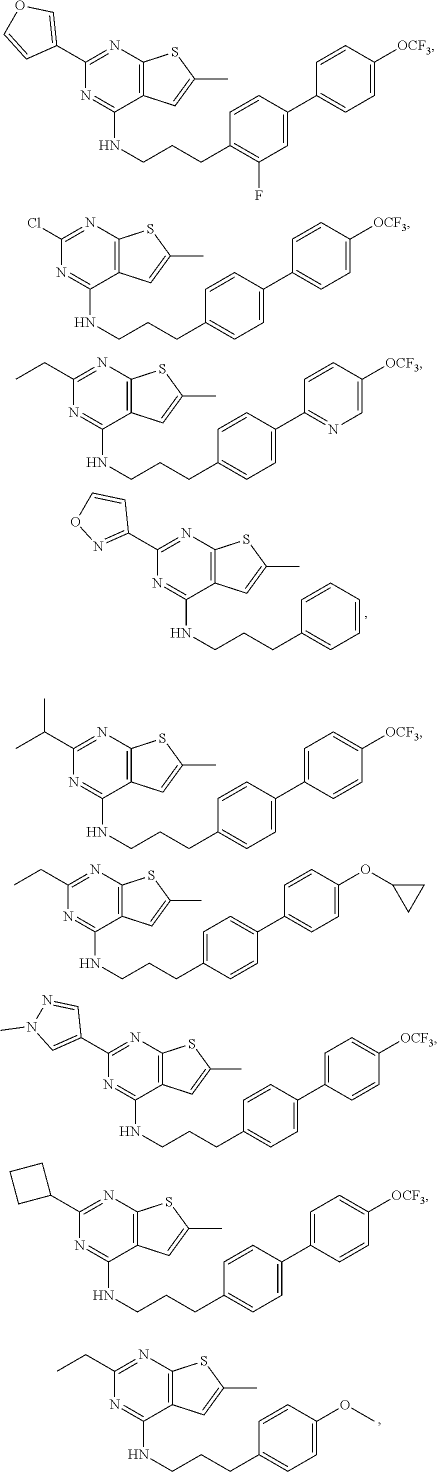

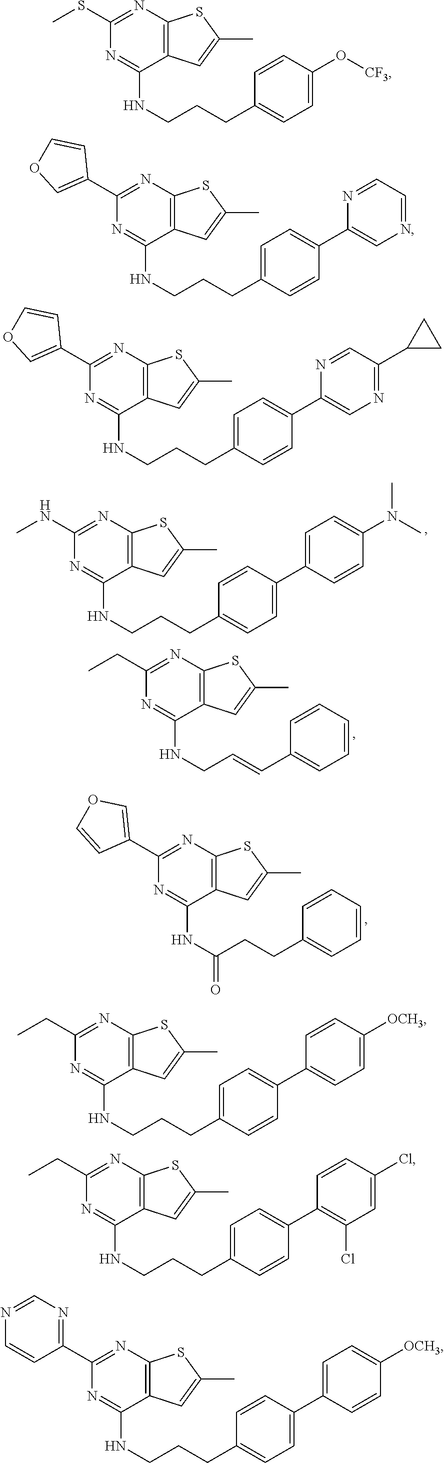

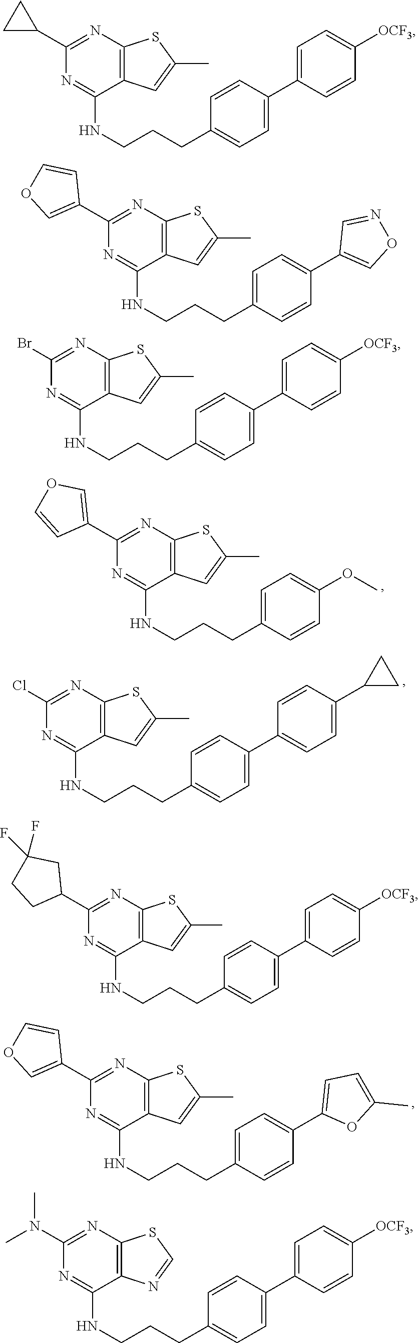

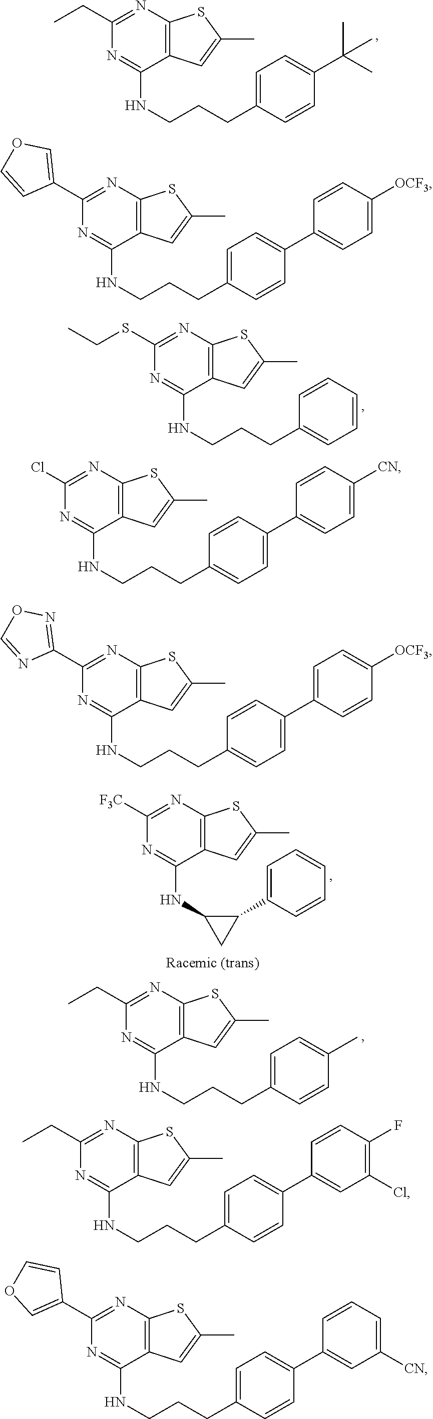

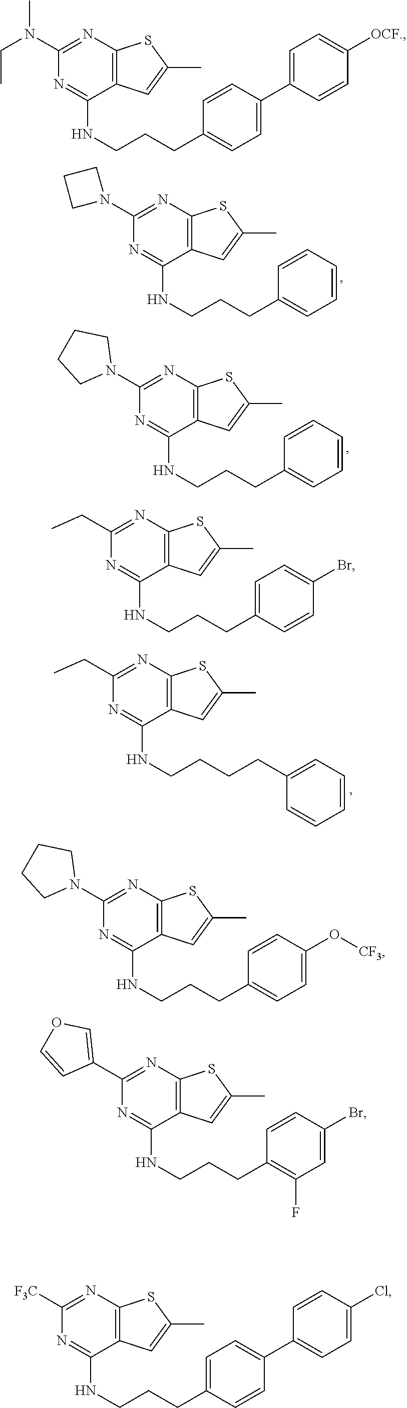

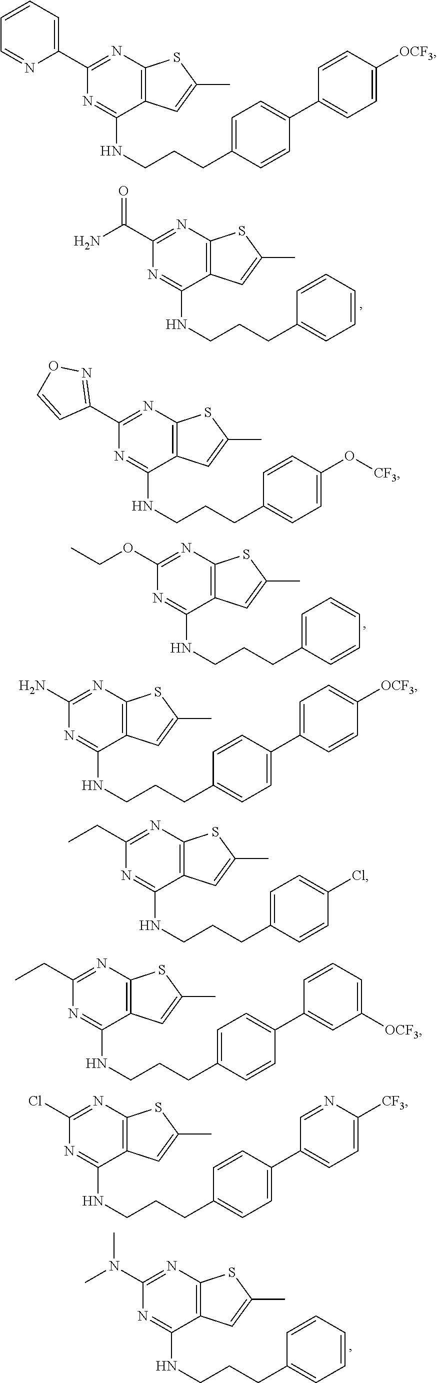

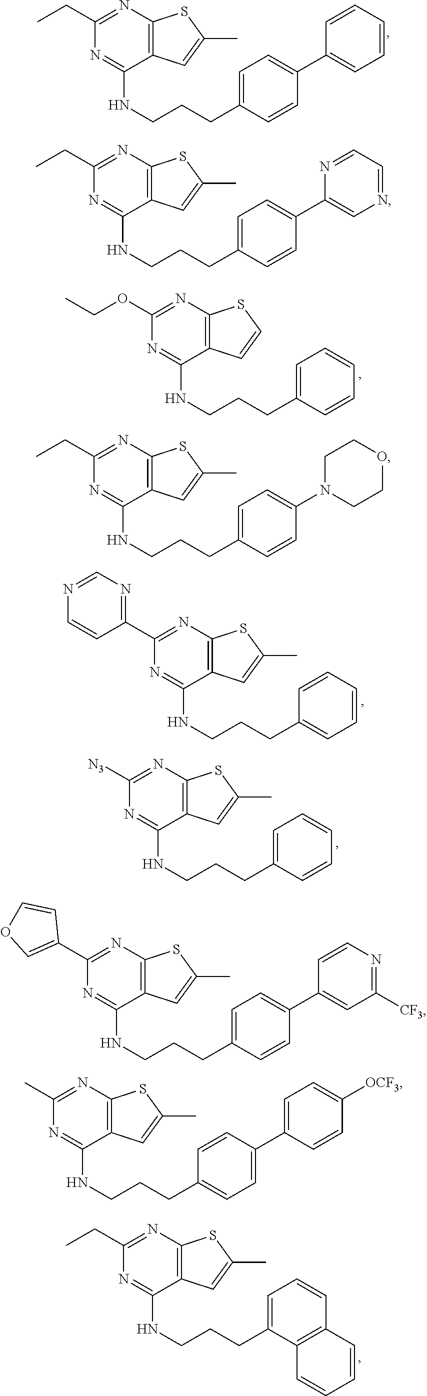

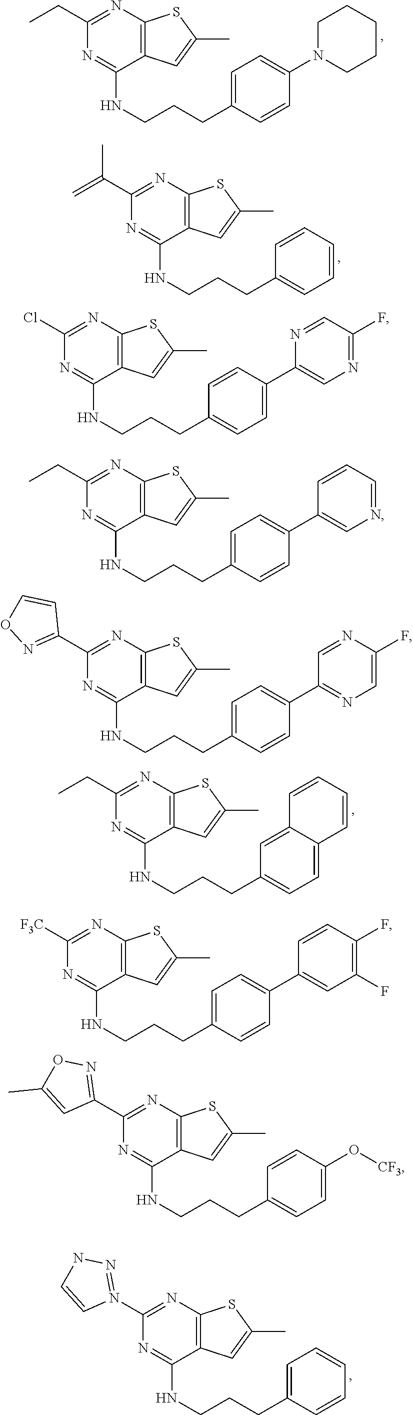

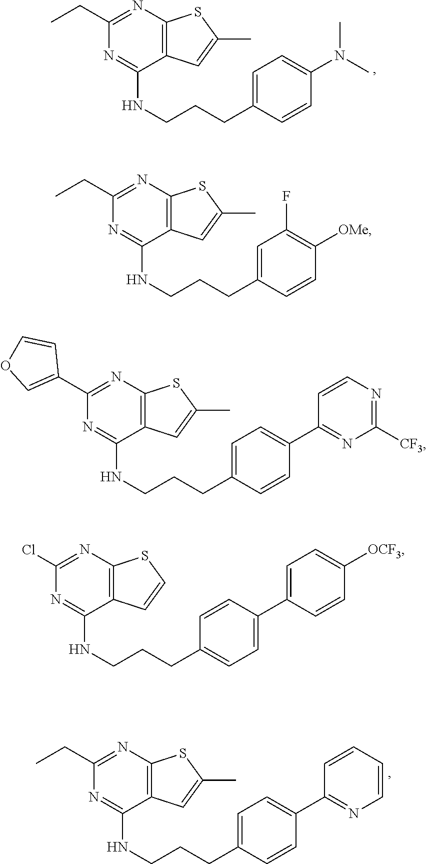

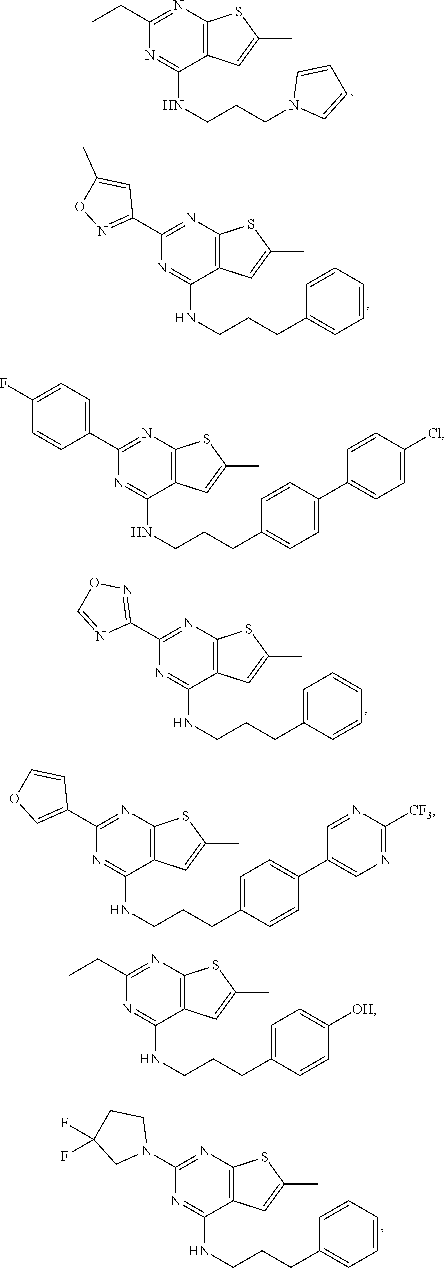

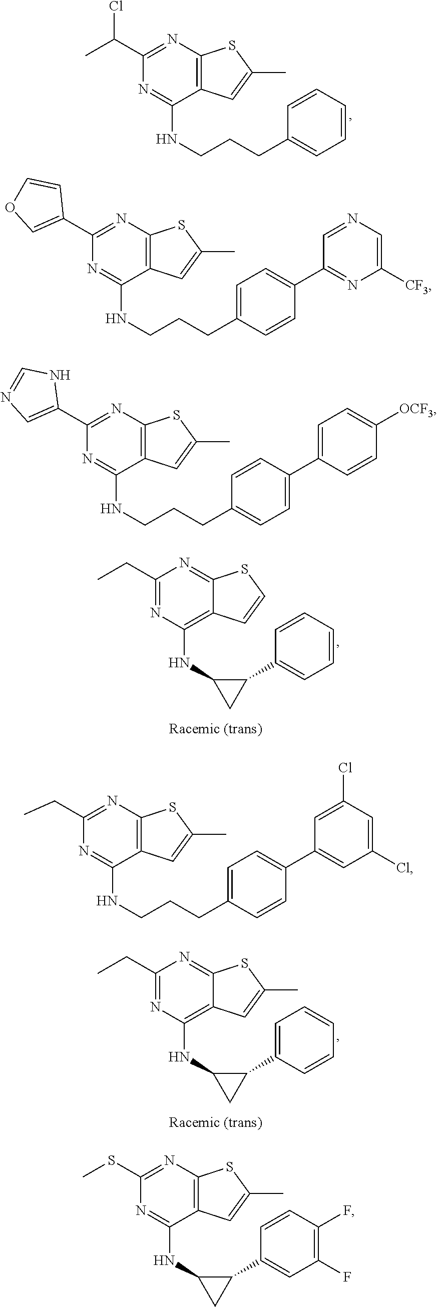

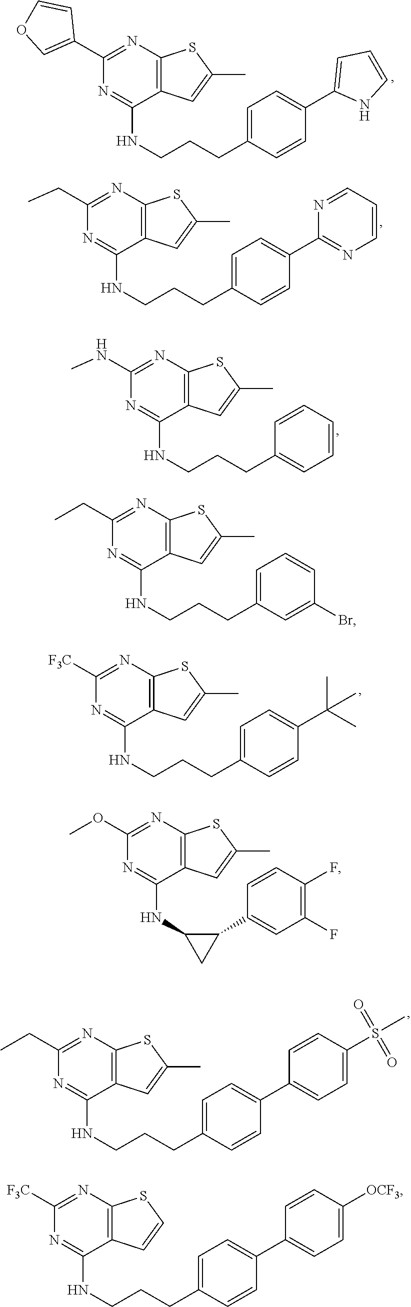

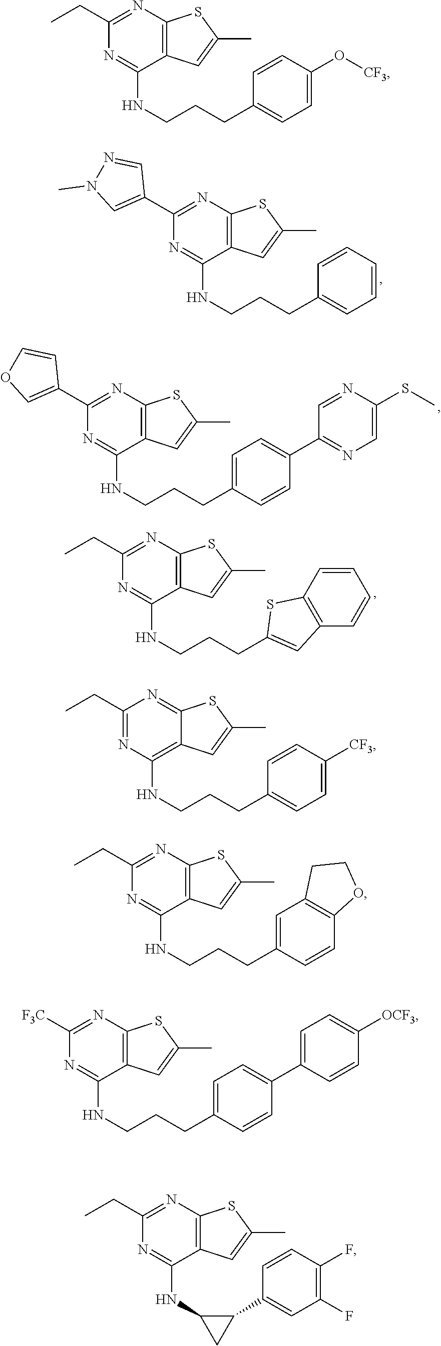

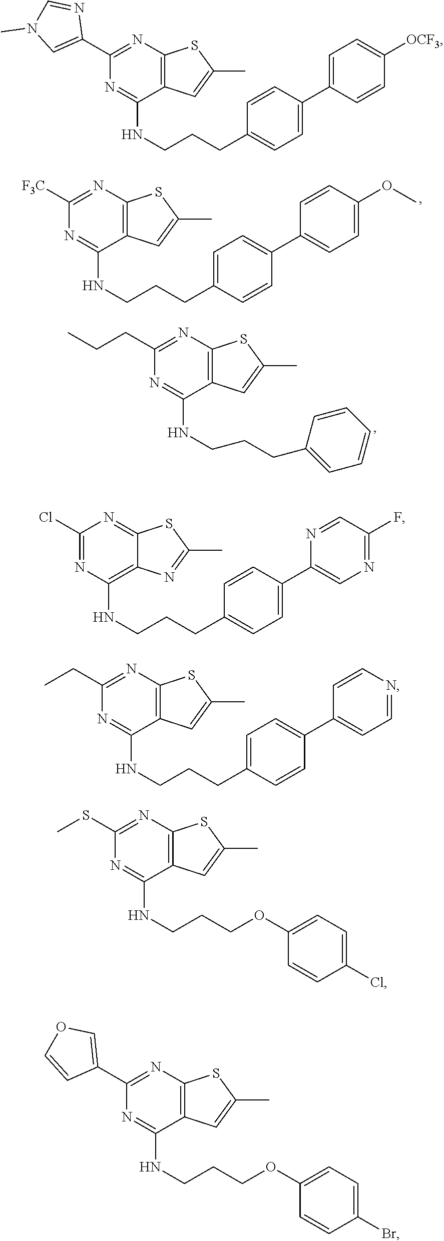

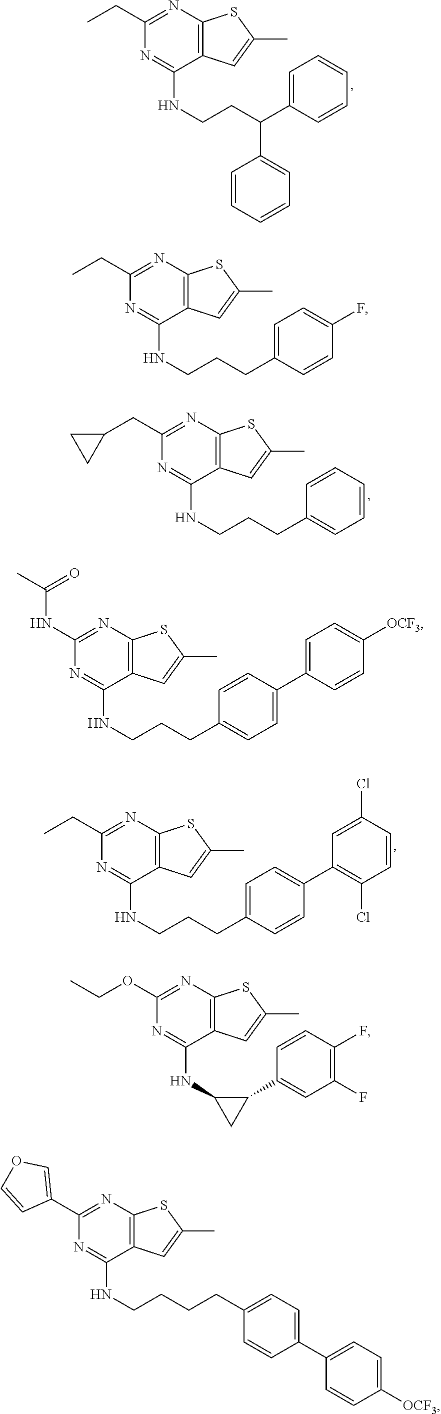

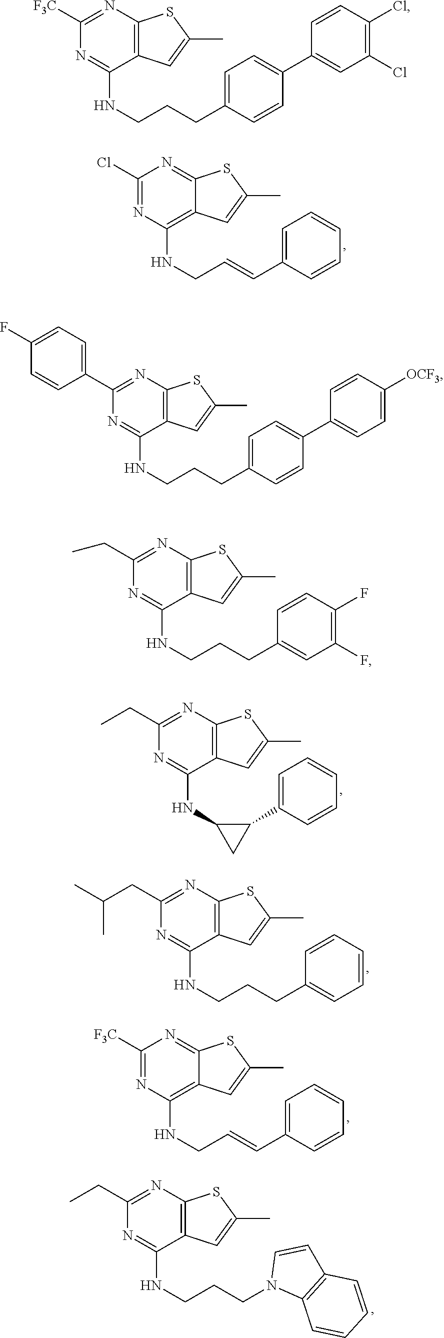

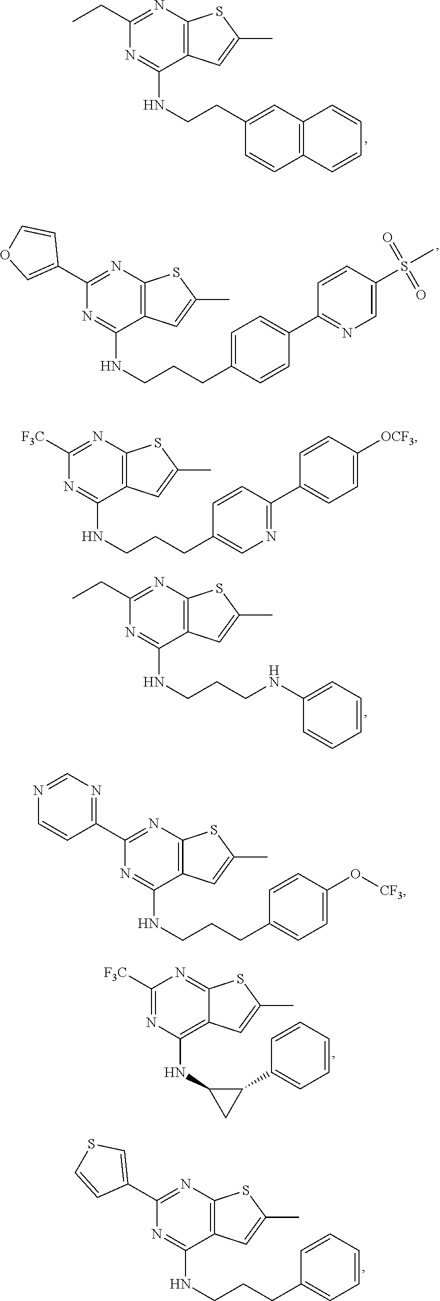

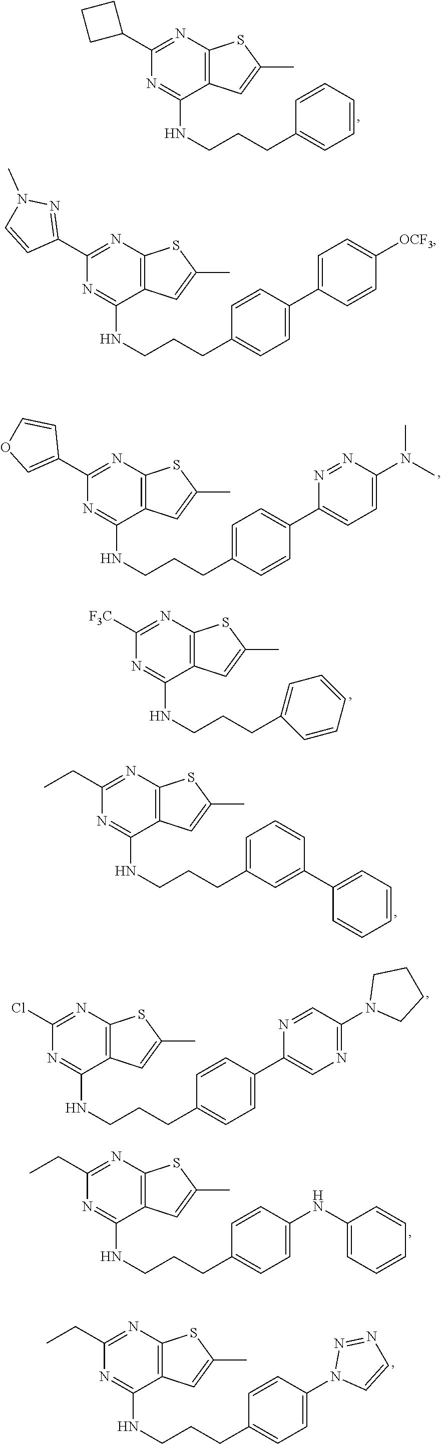

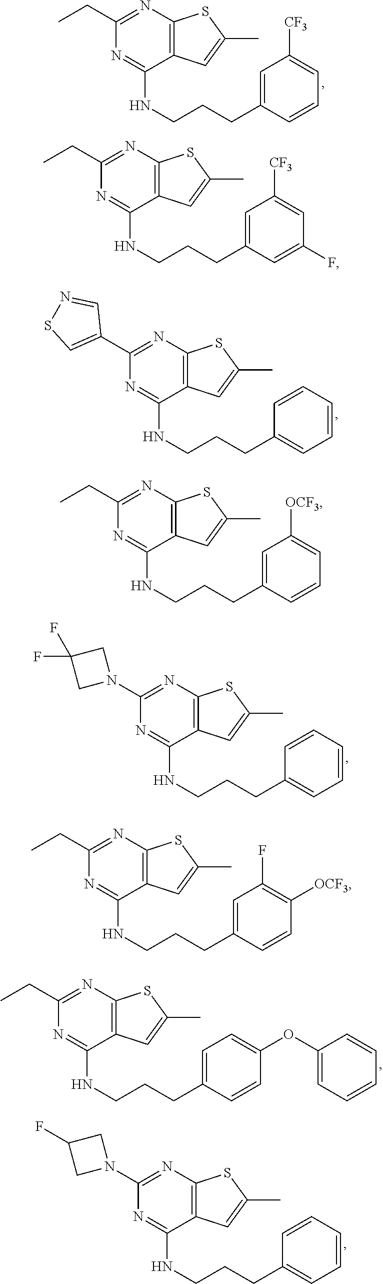

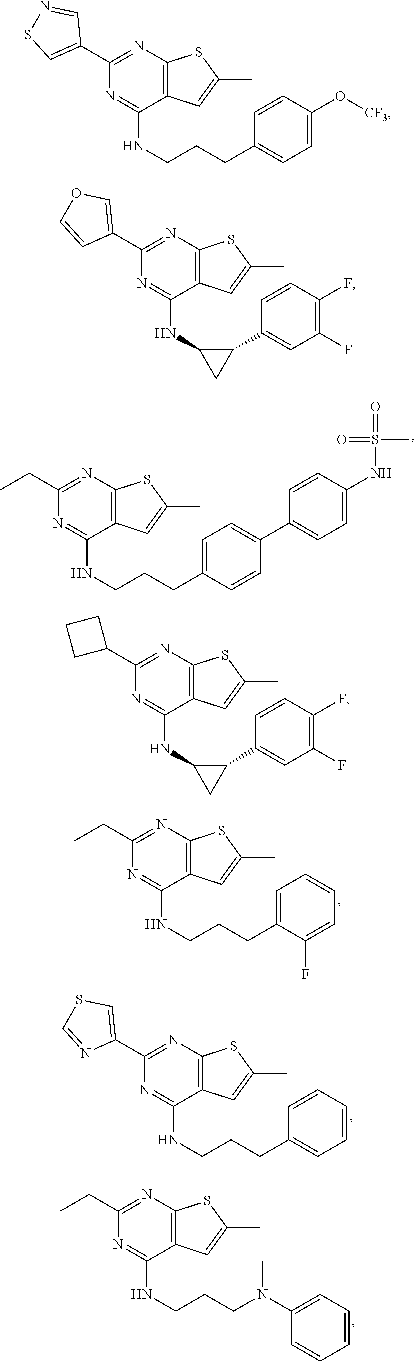

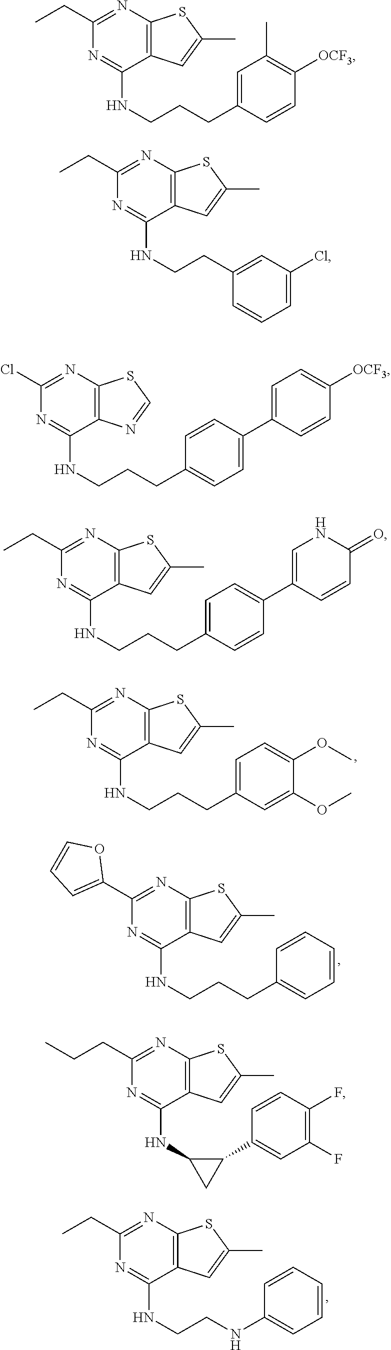

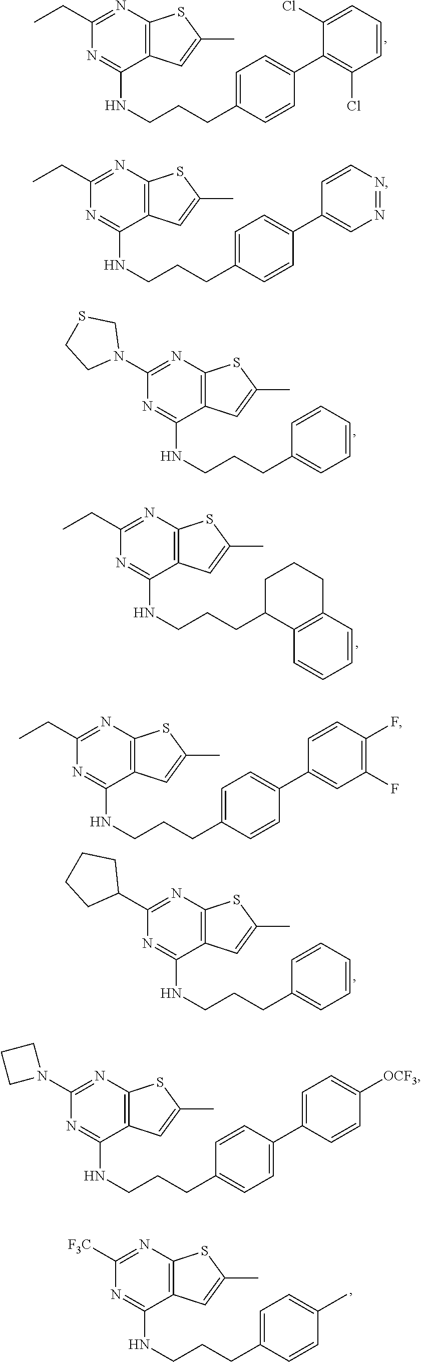

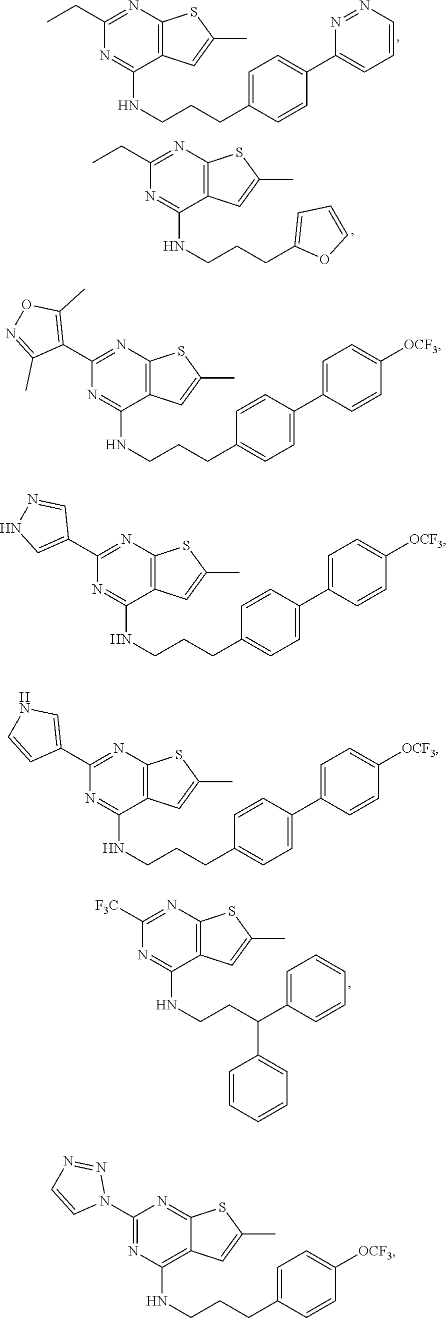

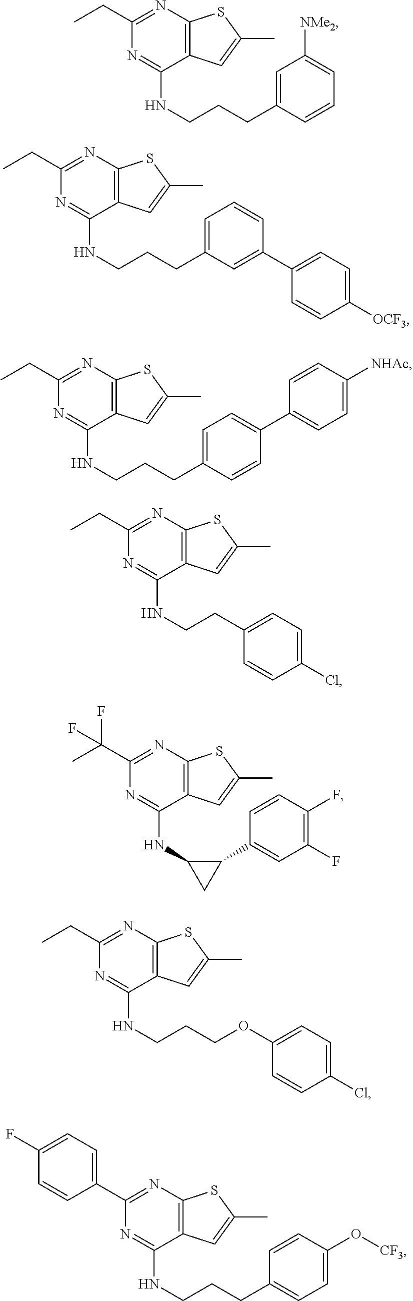

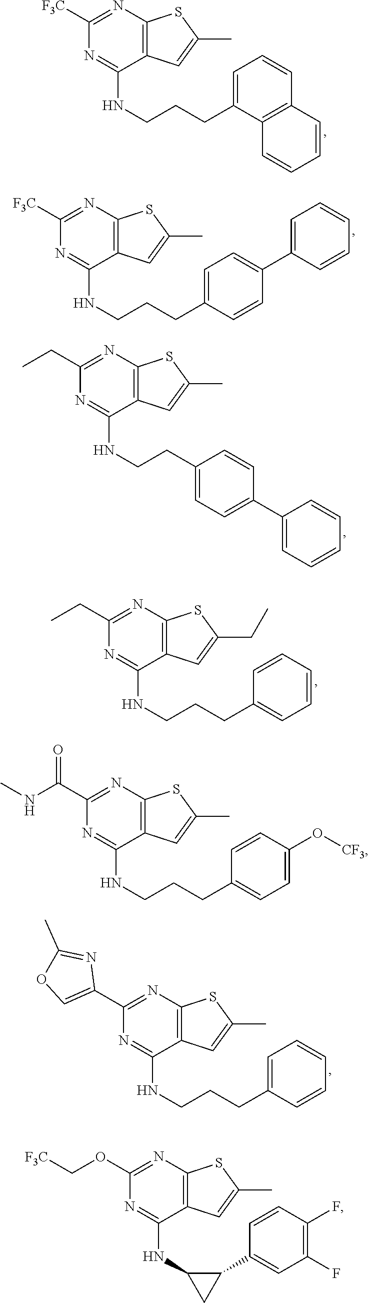

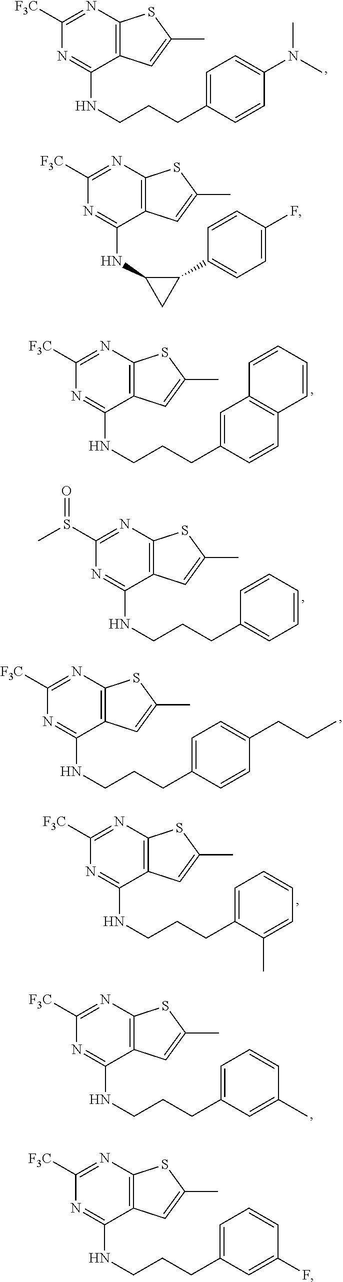

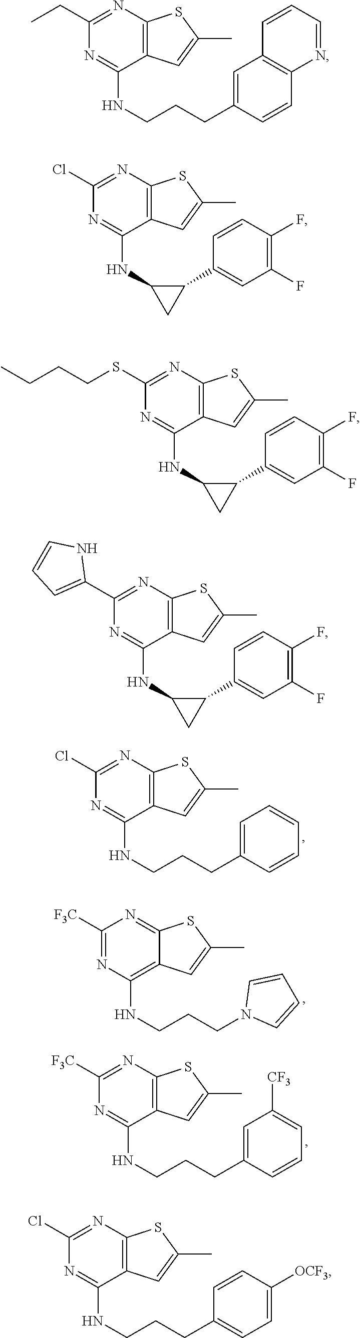

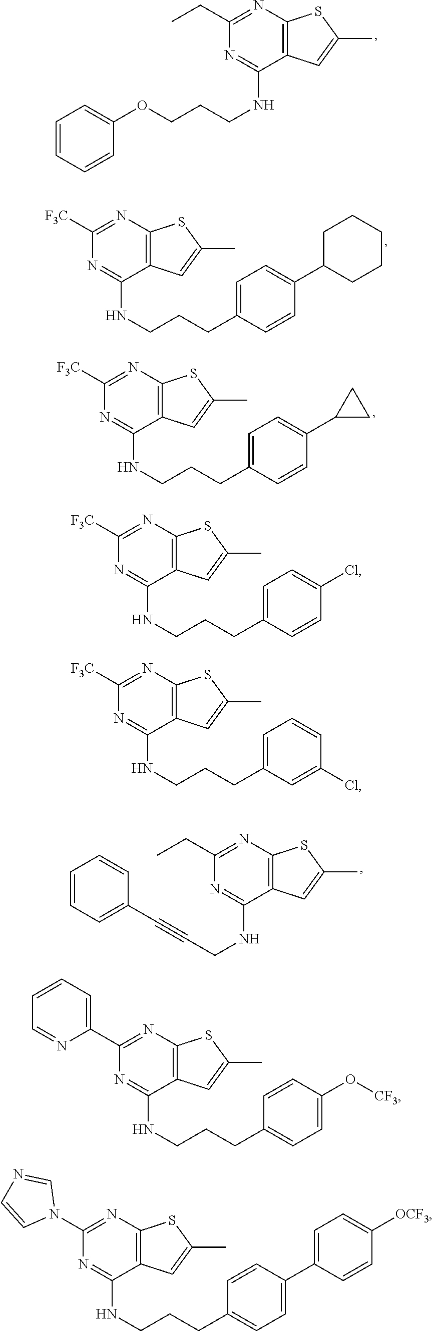

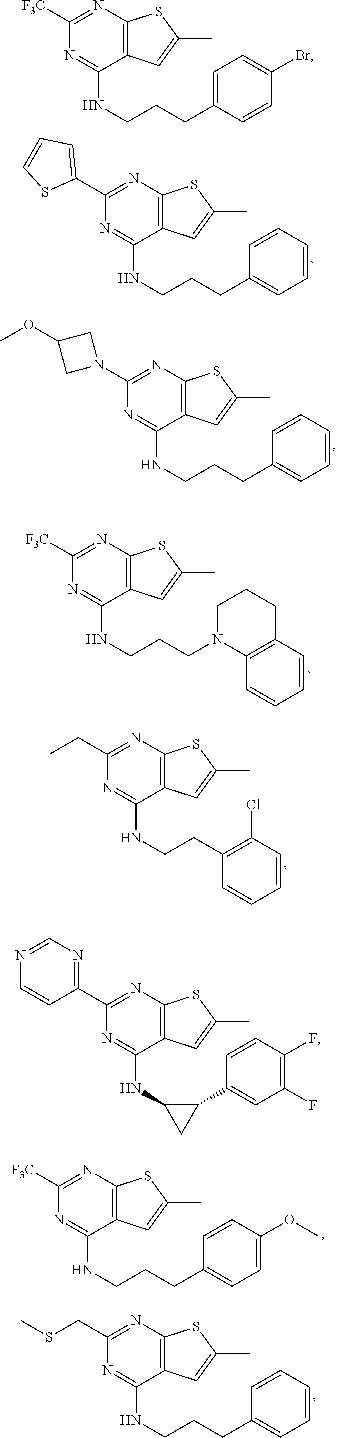

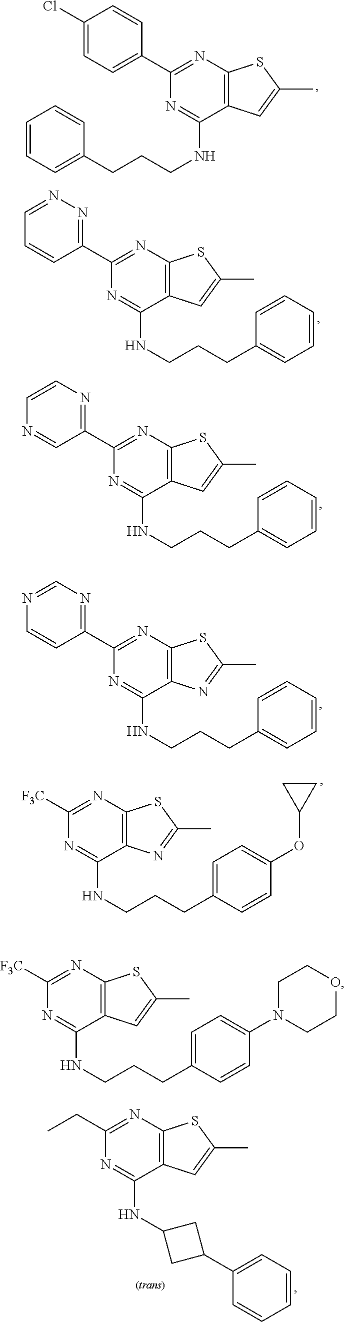

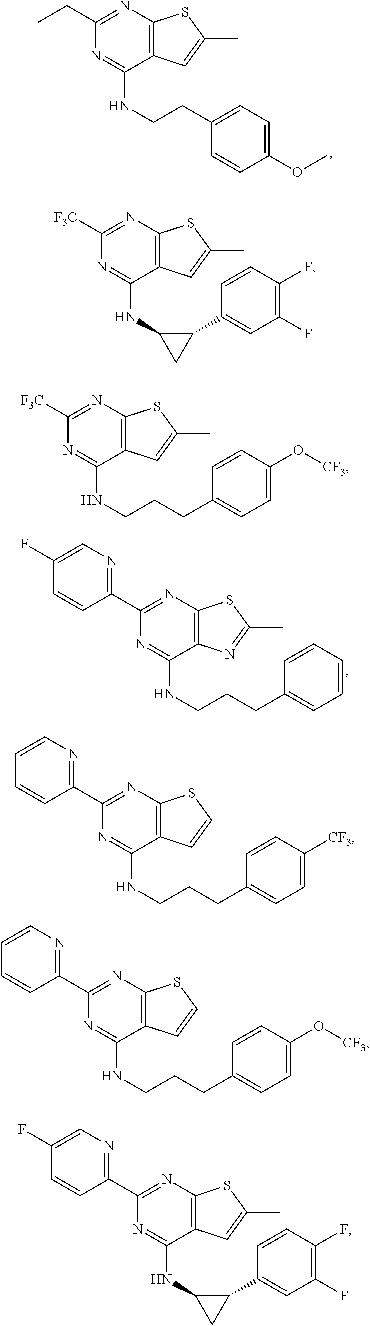

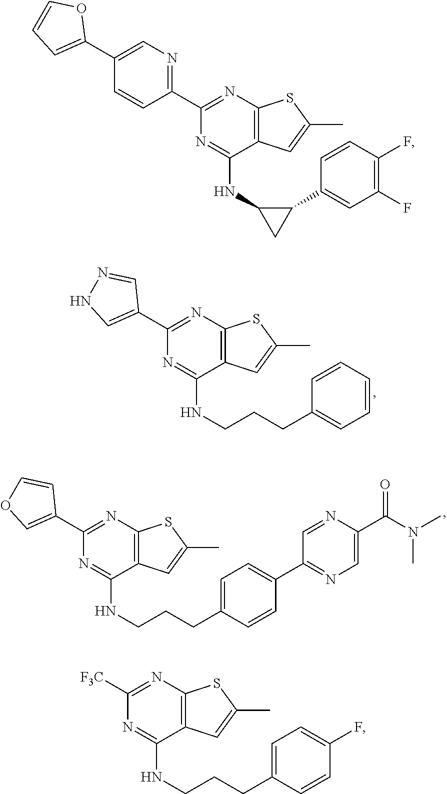 [SMILES: Cc1cc2c(N[C@@H]3C[C@H]3c3ccc(F)c(F)c3)nc(-c3ccc(-c4ccco4)cn3)nc2s1 ; Cc1cc2c(NCCCc3ccccc3)nc(-c3cn[nH]c3)nc2s1 ; Cc1cc2c(NCCCc3ccc(-c4cnc(C(=O)N(C)C)cn4)cc3)nc(-c3ccoc3)nc2s1 ; Cc1cc2c(NCCCc3ccc(F)cc3)nc(C(F)(F)F)nc2s1]

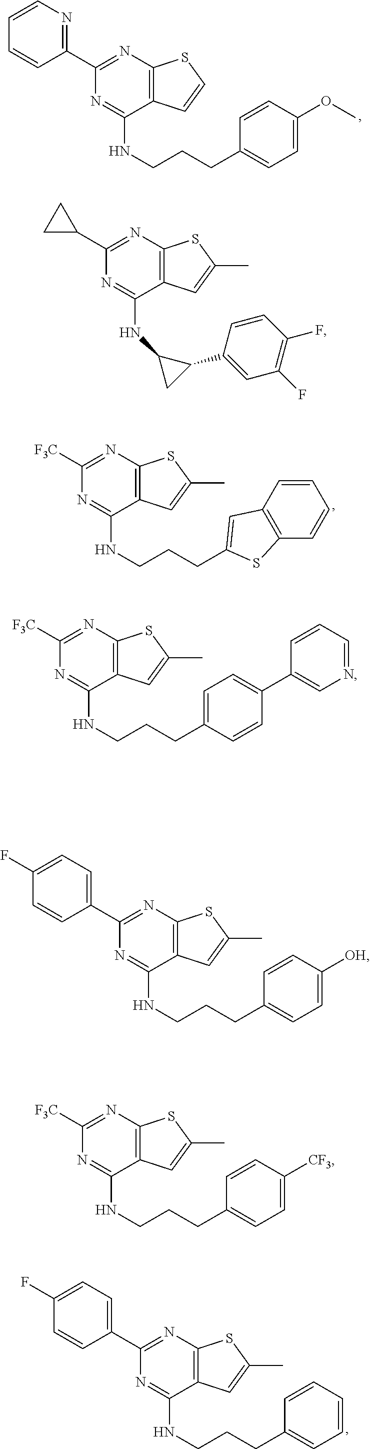

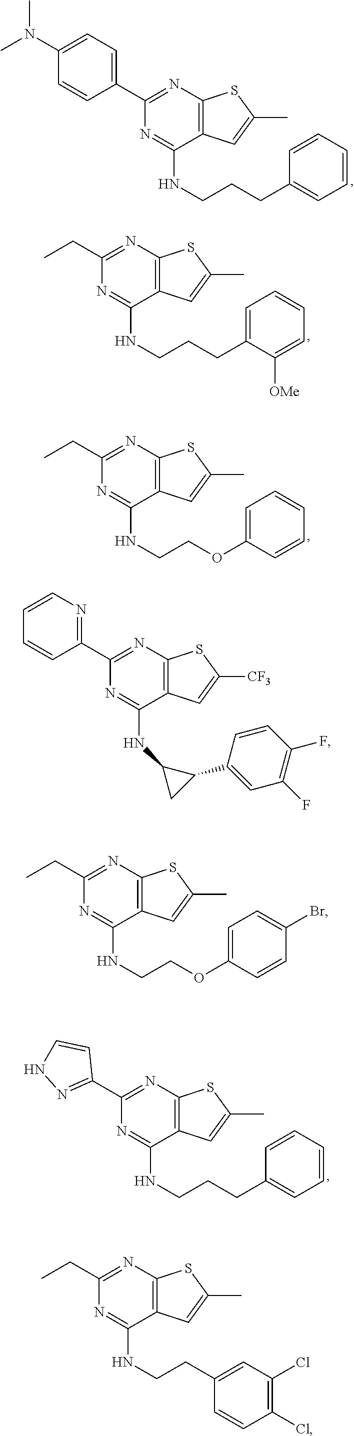

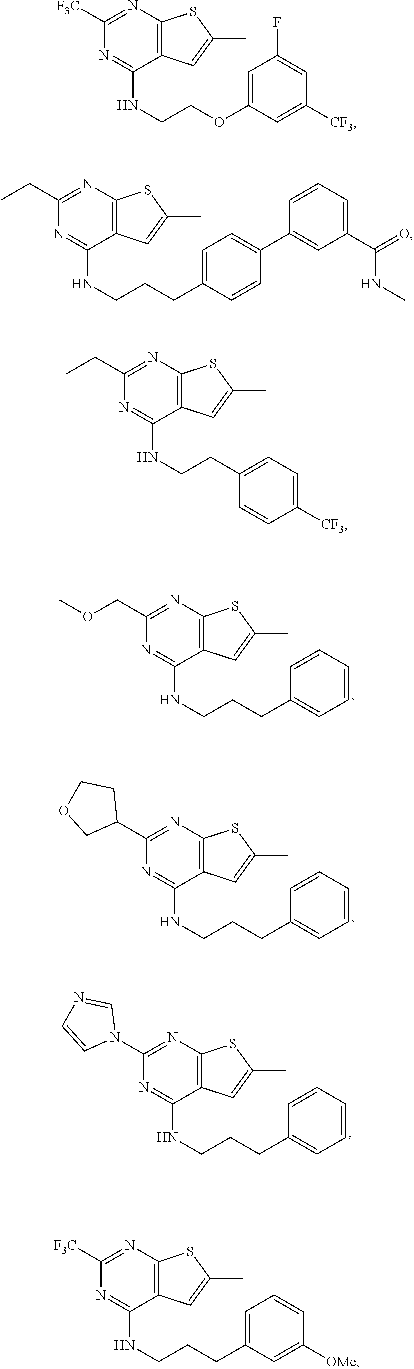

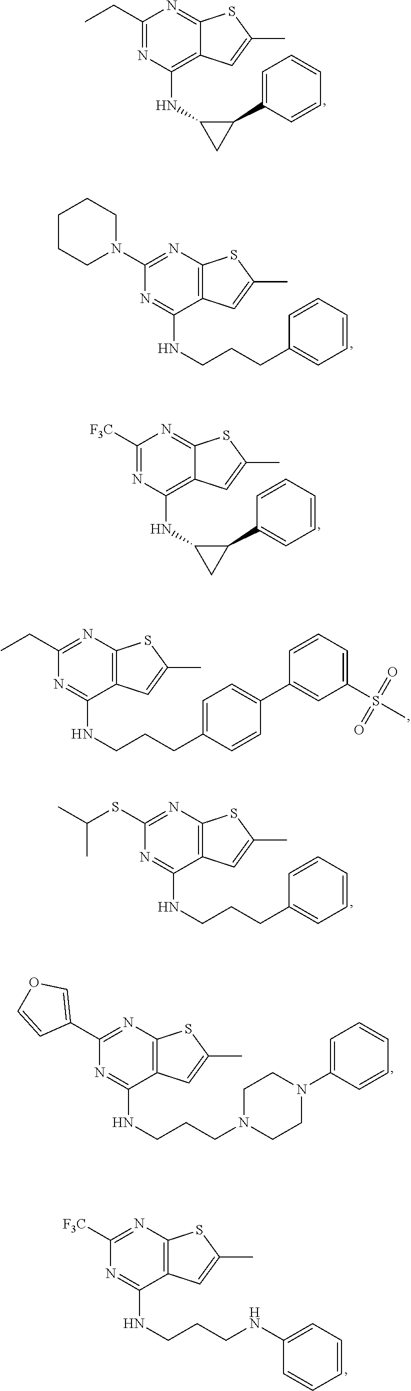

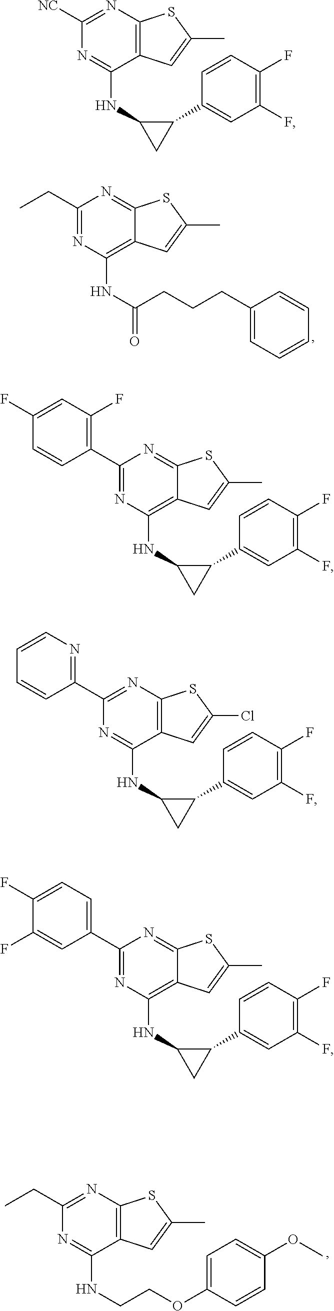 [SMILES: Cc1cc2c(N[C@@H]3C[C@H]3c3ccc(F)c(F)c3)nc(C#N)nc2s1 ; CCc1nc(NC(=O)CCCc2ccccc2)c2cc(C)sc2n1 ; Cc1cc2c(N[C@@H]3C[C@H]3c3ccc(F)c(F)c3)nc(-c3ccc(F)cc3F)nc2s1 ; Fc1ccc([C@@H]2C[C@H]2Nc2nc(-c3ccccn3)nc3sc(Cl)cc23)cc1F ; Cc1cc2c(N[C@@H]3C[C@H]3c3ccc(F)c(F)c3)nc(-c3ccc(F)c(F)c3)nc2s1 ; CCc1nc(NCCOc2ccc(OC)cc2)c2cc(C)sc2n1]

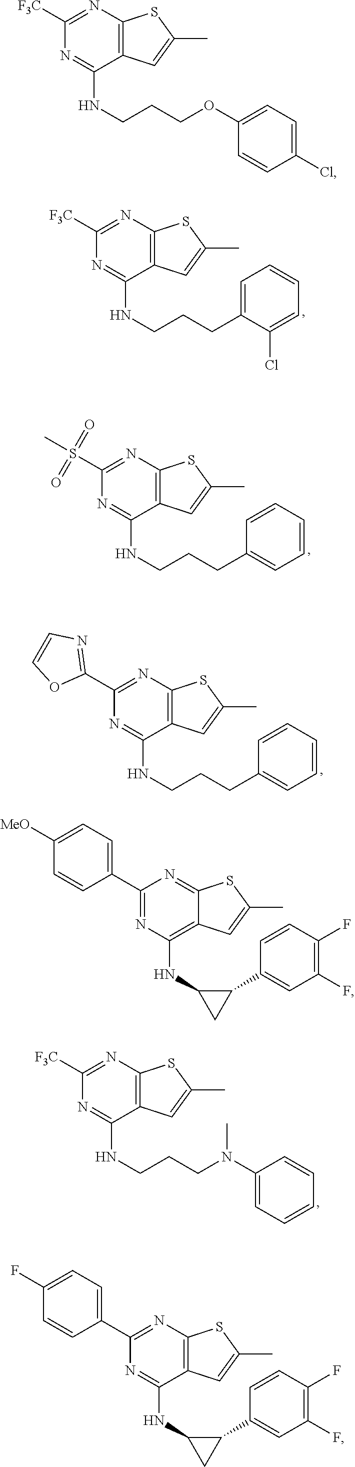

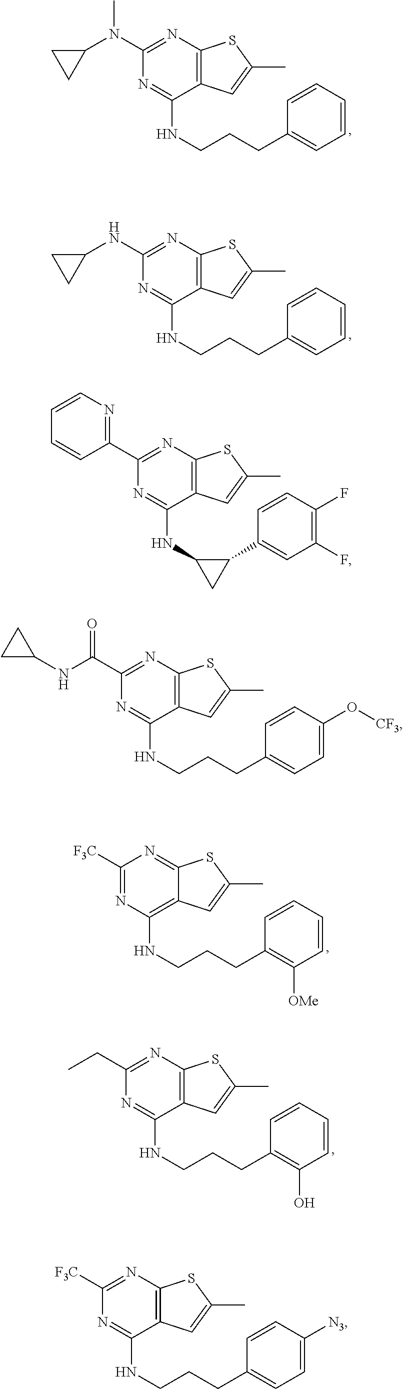

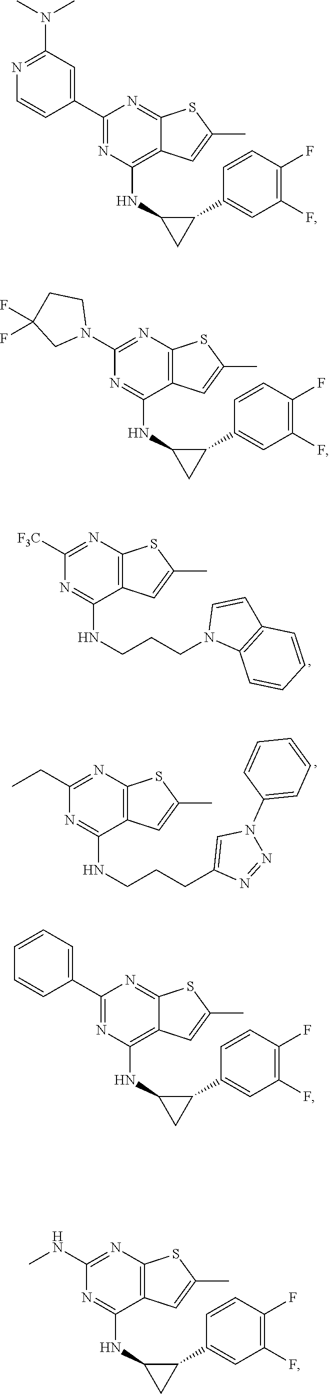 [SMILES: Cc1cc2c(N[C@@H]3C[C@H]3c3ccc(F)c(F)c3)nc(-c3ccnc(N(C)C)c3)nc2s1 ; Cc1cc2c(N[C@@H]3C[C@H]3c3ccc(F)c(F)c3)nc(N3CCC(F)(F)C3)nc2s1 ; Cc1cc2c(NCCCn3ccc4ccccc43)nc(C(F)(F)F)nc2s1 ; CCc1nc(NCCCc2cn(-c3ccccc3)nn2)c2cc(C)sc2n1 ; Cc1cc2c(N[C@@H]3C[C@H]3c3ccc(F)c(F)c3)nc(-c3ccccc3)nc2s1 ; CNc1nc(N[C@@H]2C[C@H]2c2ccc(F)c(F)c2)c2cc(C)sc2n1]

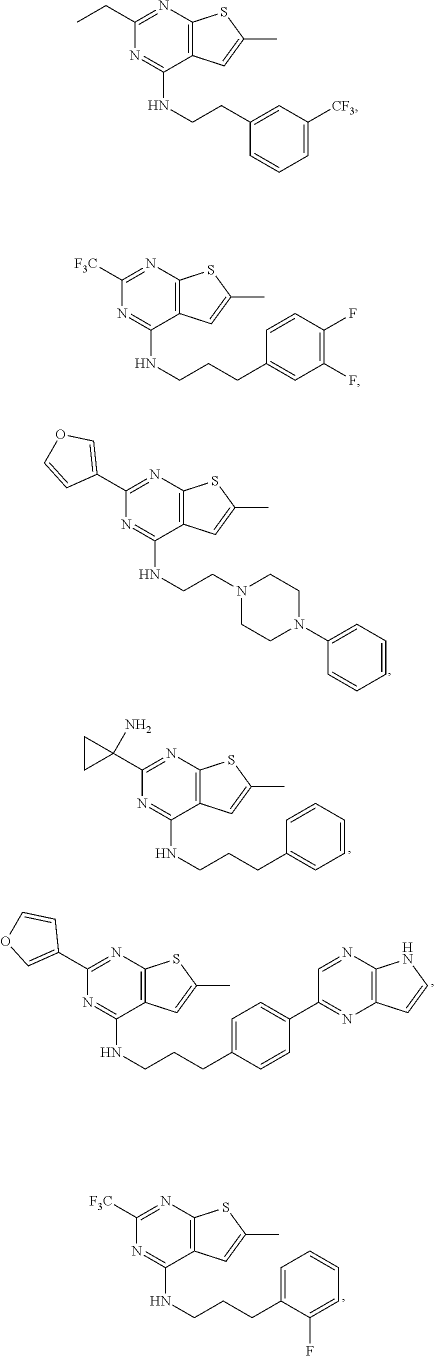 [SMILES: CCc1nc(NCCc2cccc(C(F)(F)F)c2)c2cc(C)sc2n1 ; Cc1cc2c(NCCCc3ccc(F)c(F)c3)nc(C(F)(F)F)nc2s1 ; Cc1cc2c(NCCN3CCN(c4ccccc4)CC3)nc(-c3ccoc3)nc2s1 ; Cc1cc2c(NCCCc3ccccc3)nc(C3(N)CC3)nc2s1 ; Cc1cc2c(NCCCc3ccc(-c4cnc5[nH]ccc5n4)cc3)nc(-c3ccoc3)nc2s1 ; Cc1cc2c(NCCCc3ccccc3F)nc(C(F)(F)F)nc2s1]

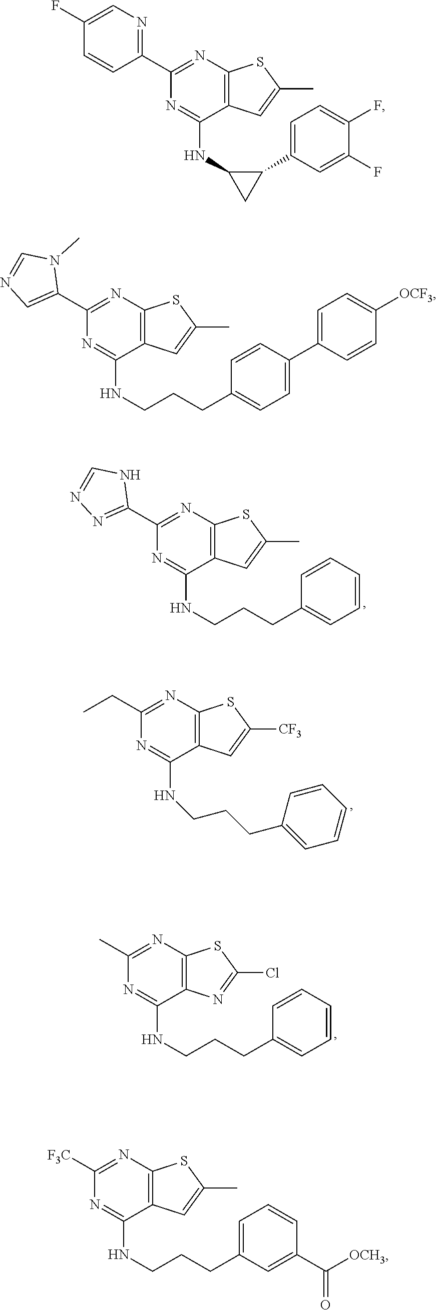 [SMILES: Cc1cc2c(N[C@@H]3C[C@H]3c3ccc(F)c(F)c3)nc(-c3ccc(F)cn3)nc2s1 ; Cc1cc2c(NCCCc3ccc(-c4ccc(OC(F)(F)F)cc4)cc3)nc(-c3cncn3C)nc2s1 ; Cc1cc2c(NCCCc3ccccc3)nc(-c3nnc[nH]3)nc2s1 ; CCc1nc(NCCCc2ccccc2)c2cc(C(F)(F)F)sc2n1 ; Cc1nc(NCCCc2ccccc2)c2nc(Cl)sc2n1 ; COC(=O)c1cccc(CCCNc2nc(C(F)(F)F)nc3sc(C)cc23)c1]

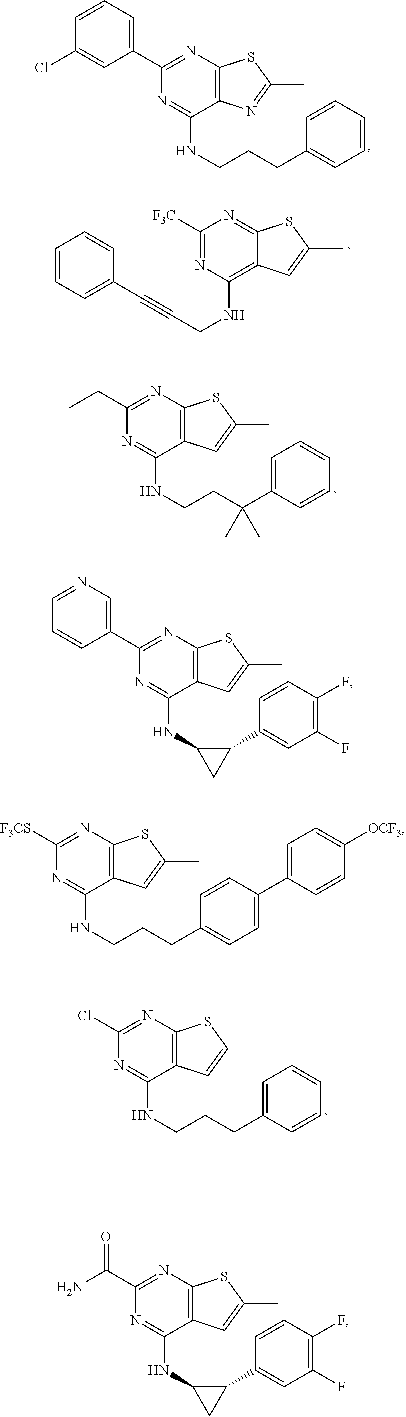

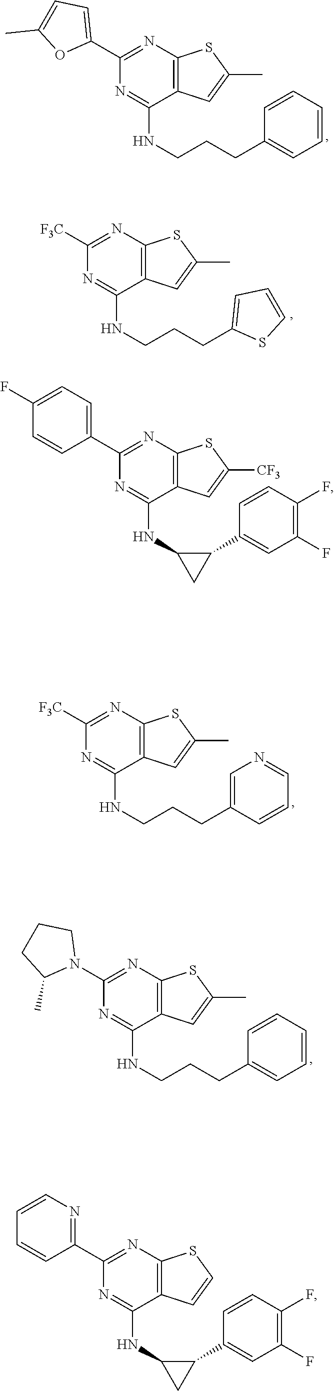 [SMILES: Cc1ccc(-c2nc(NCCCc3ccccc3)c3cc(C)sc3n2)o1 ; Cc1cc2c(NCCCc3cccs3)nc(C(F)(F)F)nc2s1 ; Fc1ccc(-c2nc(N[C@@H]3C[C@H]3c3ccc(F)c(F)c3)c3cc(C(F)(F)F)sc3n2)cc1 ; Cc1cc2c(NCCCc3cccnc3)nc(C(F)(F)F)nc2s1 ; Cc1cc2c(NCCCc3ccccc3)nc(N3CCC[C@H]3C)nc2s1 ; Fc1ccc([C@@H]2C[C@H]2Nc2nc(-c3ccccn3)nc3sccc23)cc1F]

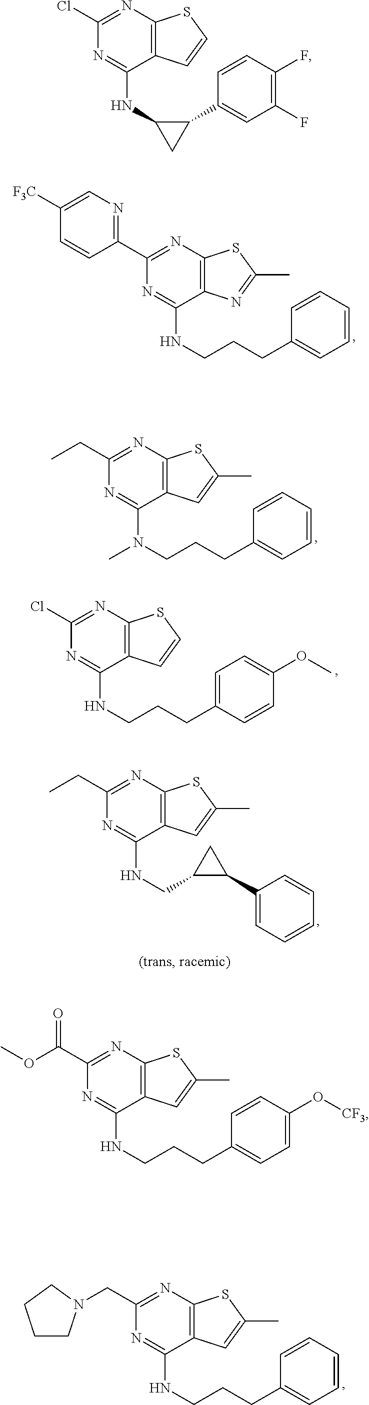

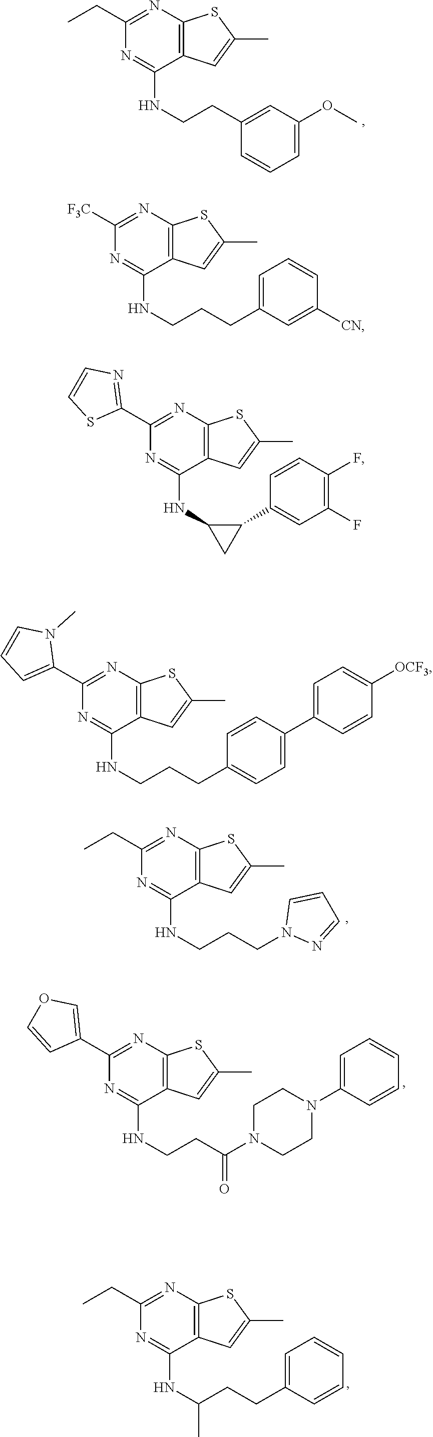

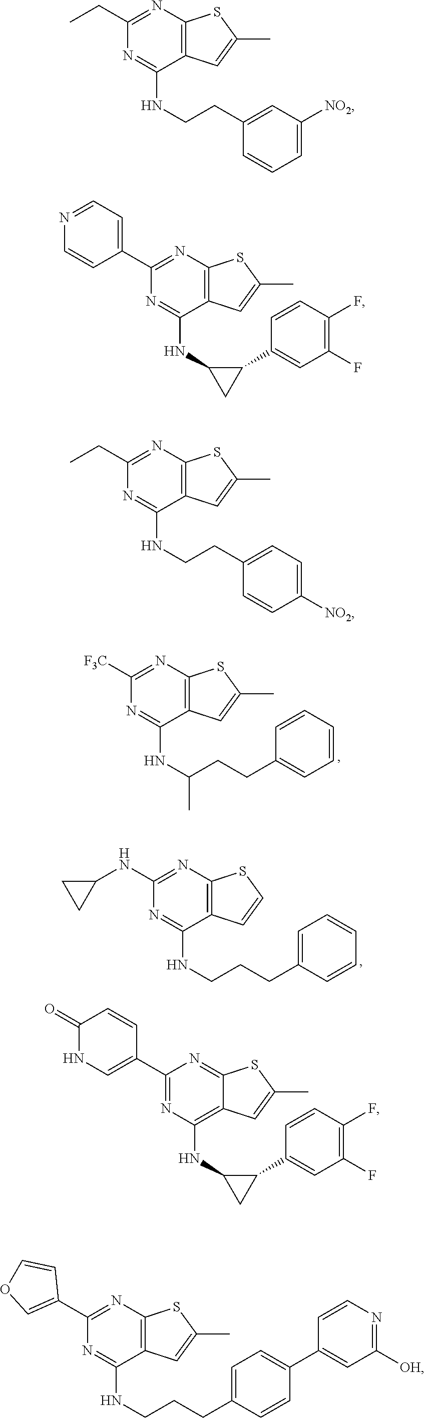

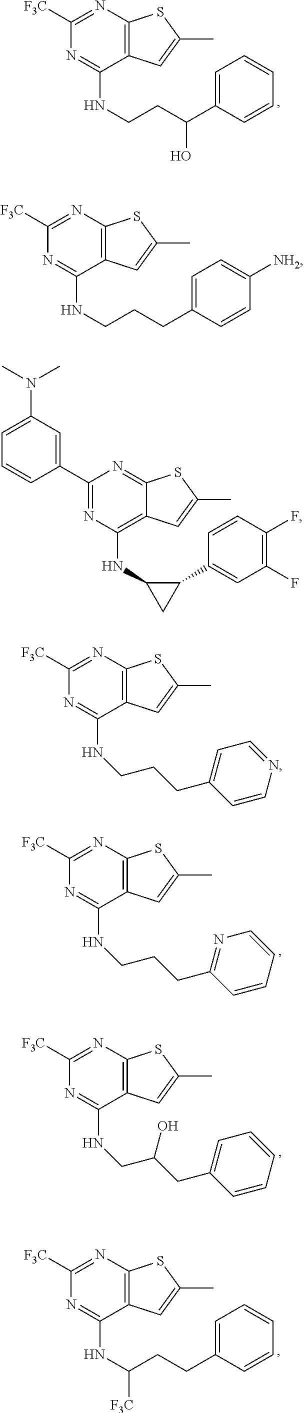

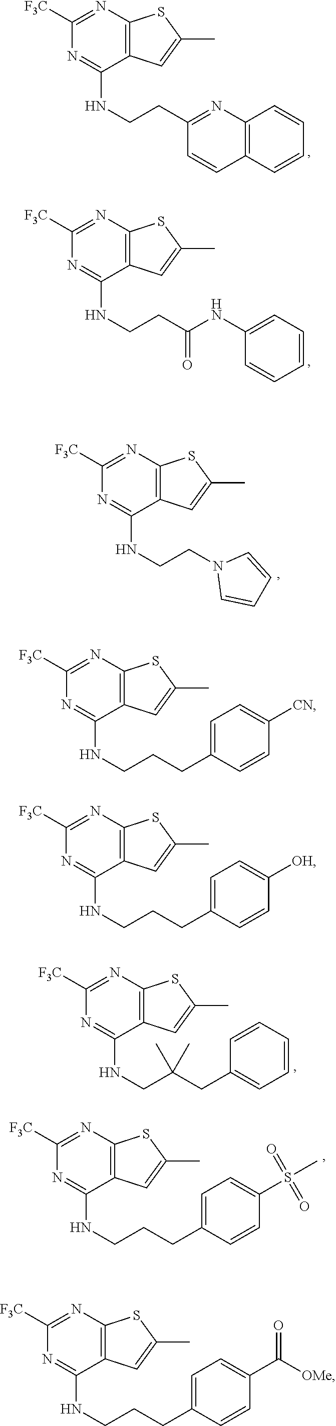

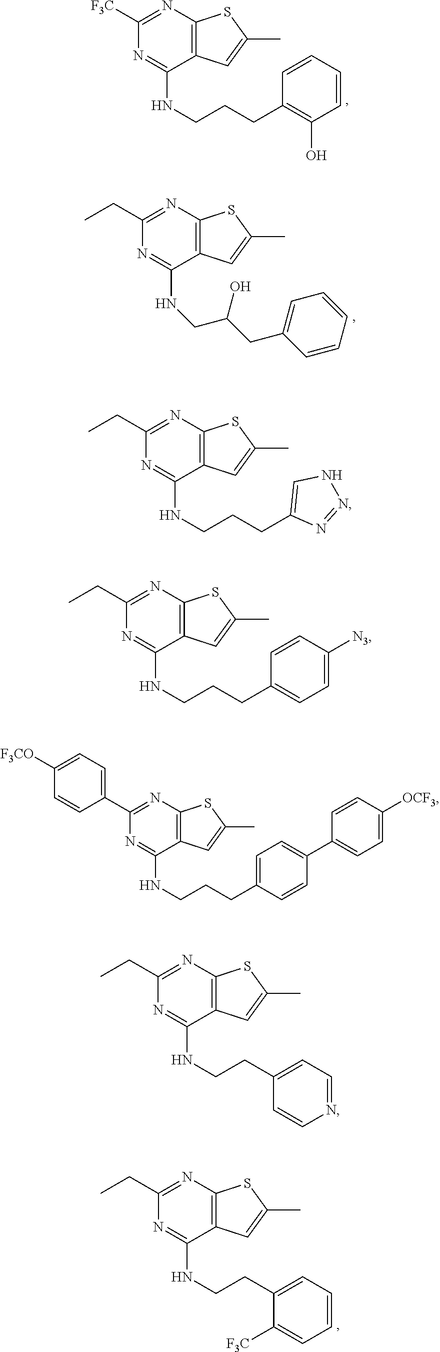

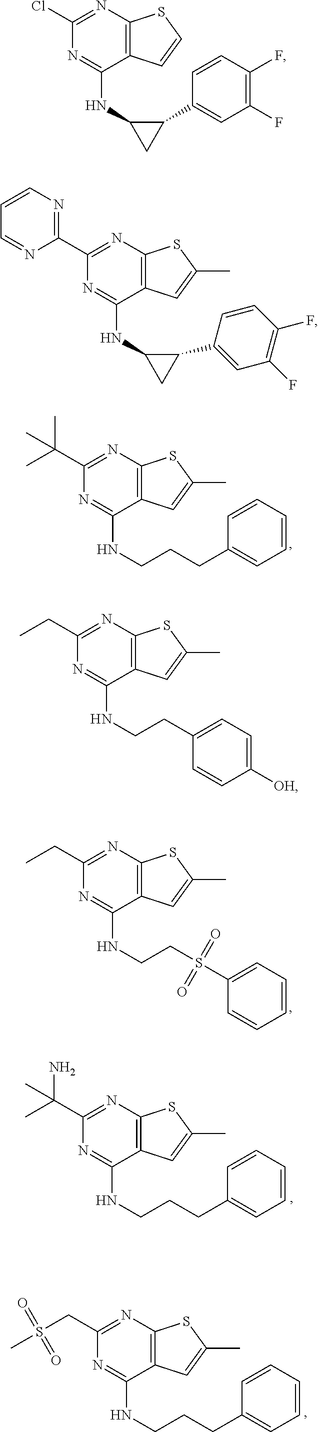

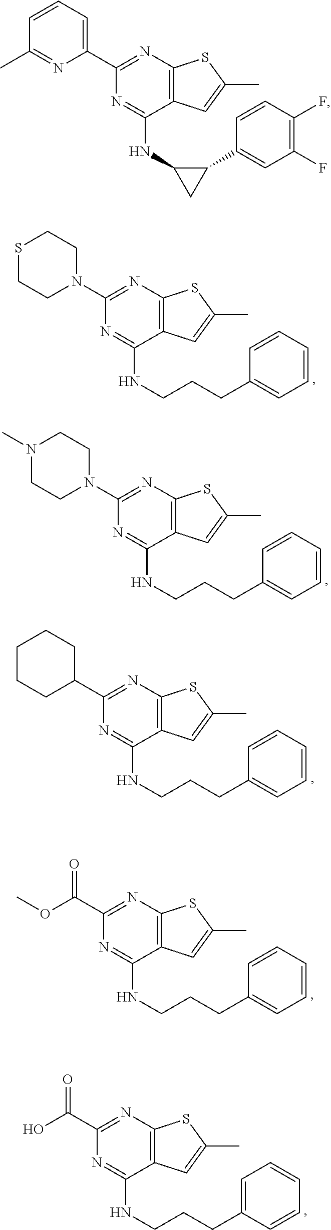 [SMILES: Cc1cccc(-c2nc(N[C@@H]3C[C@H]3c3ccc(F)c(F)c3)c3cc(C)sc3n2)n1 ; Cc1cc2c(NCCCc3ccccc3)nc(N3CCSCC3)nc2s1 ; Cc1cc2c(NCCCc3ccccc3)nc(N3CCN(C)CC3)nc2s1 ; Cc1cc2c(NCCCc3ccccc3)nc(C3CCCCC3)nc2s1 ; COC(=O)c1nc(NCCCc2ccccc2)c2cc(C)sc2n1 ; Cc1cc2c(NCCCc3ccccc3)nc(C(=O)O)nc2s1]

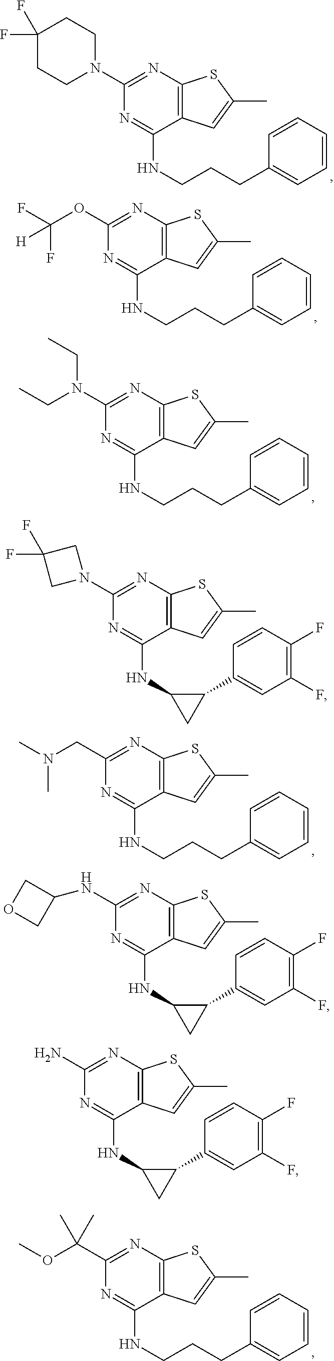

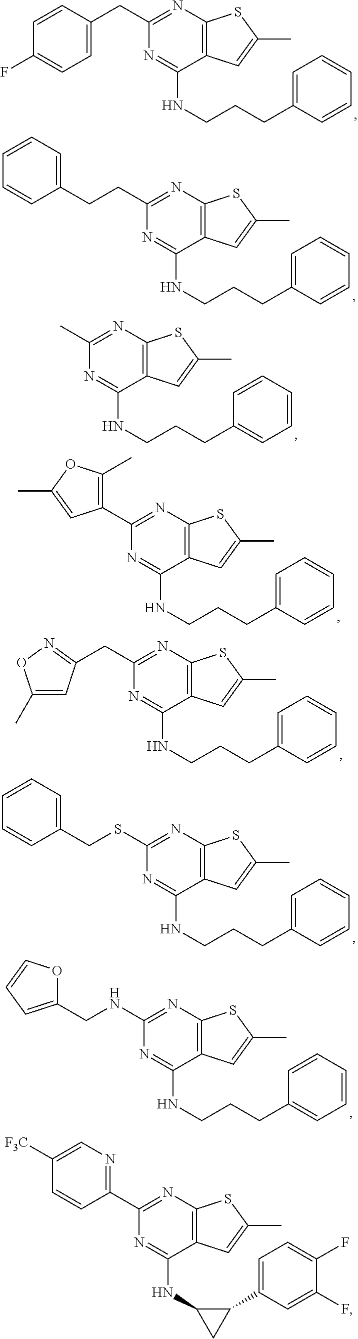

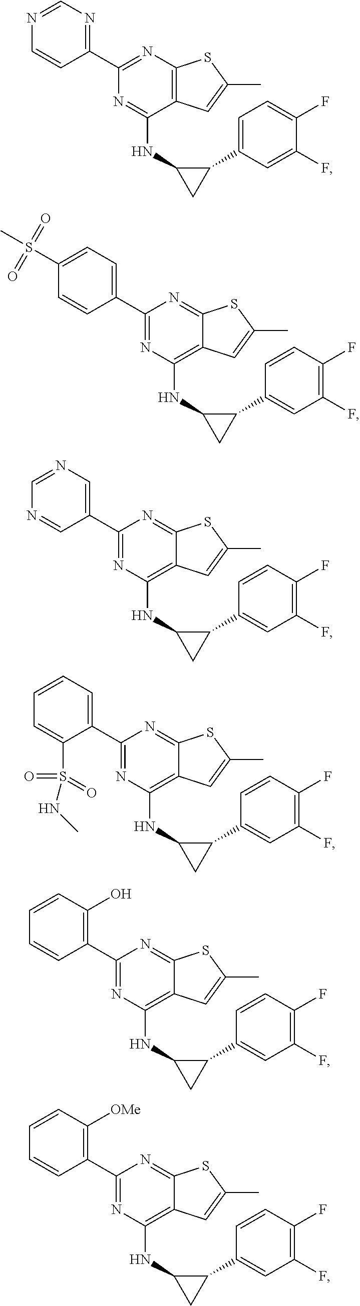 [SMILES: Cc1cc2c(N[C@@H]3C[C@H]3c3ccc(F)c(F)c3)nc(-c3ccncn3)nc2s1 ; Cc1cc2c(N[C@@H]3C[C@H]3c3ccc(F)c(F)c3)nc(-c3ccc(S(C)(=O)=O)cc3)nc2s1 ; Cc1cc2c(N[C@@H]3C[C@H]3c3ccc(F)c(F)c3)nc(-c3cncnc3)nc2s1 ; CNS(=O)(=O)c1ccccc1-c1nc(N[C@@H]2C[C@H]2c2ccc(F)c(F)c2)c2cc(C)sc2n1 ; Cc1cc2c(N[C@@H]3C[C@H]3c3ccc(F)c(F)c3)nc(-c3ccccc3O)nc2s1 ; COc1ccccc1-c1nc(N[C@@H]2C[C@H]2c2ccc(F)c(F)c2)c2cc(C)sc2n1]

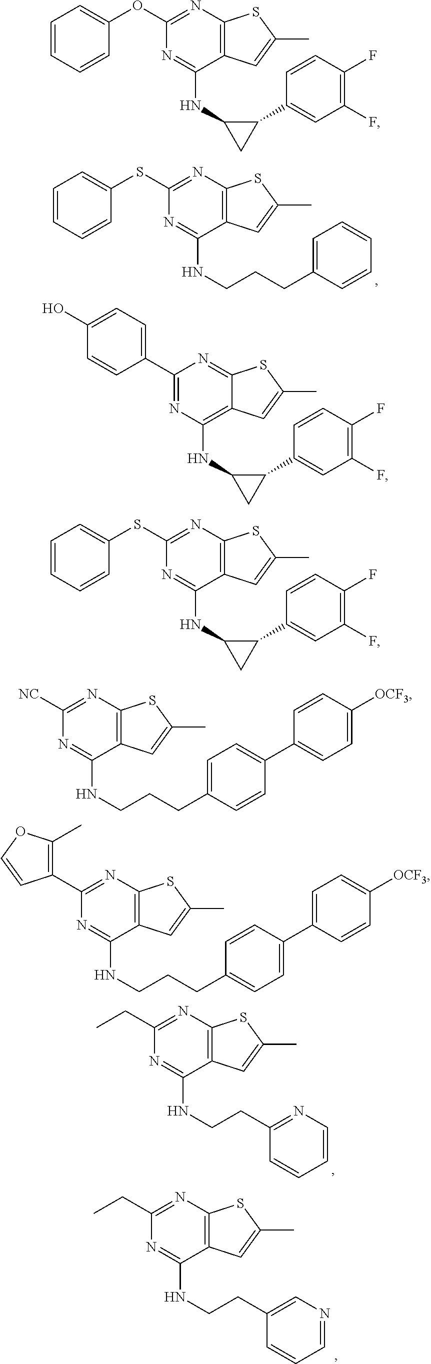

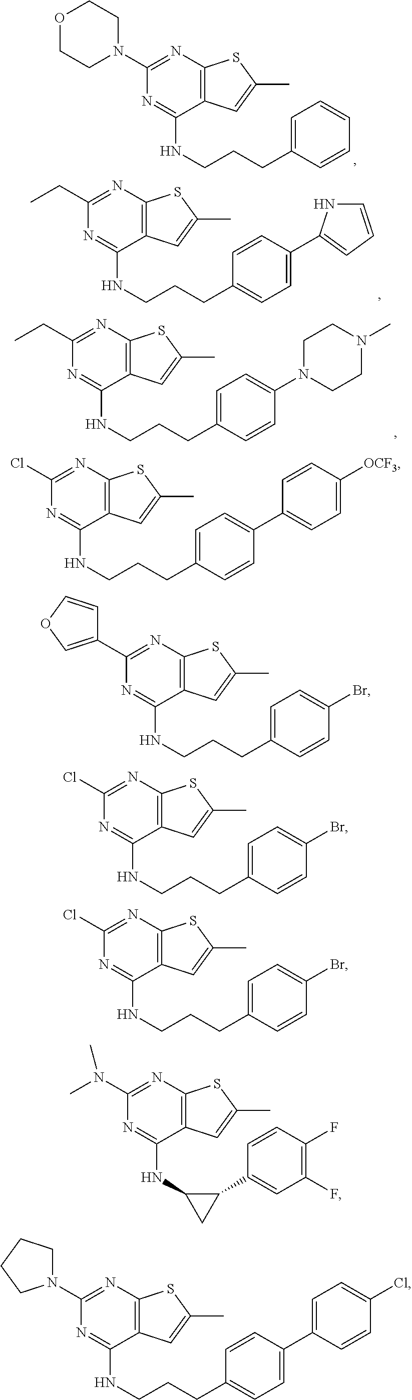

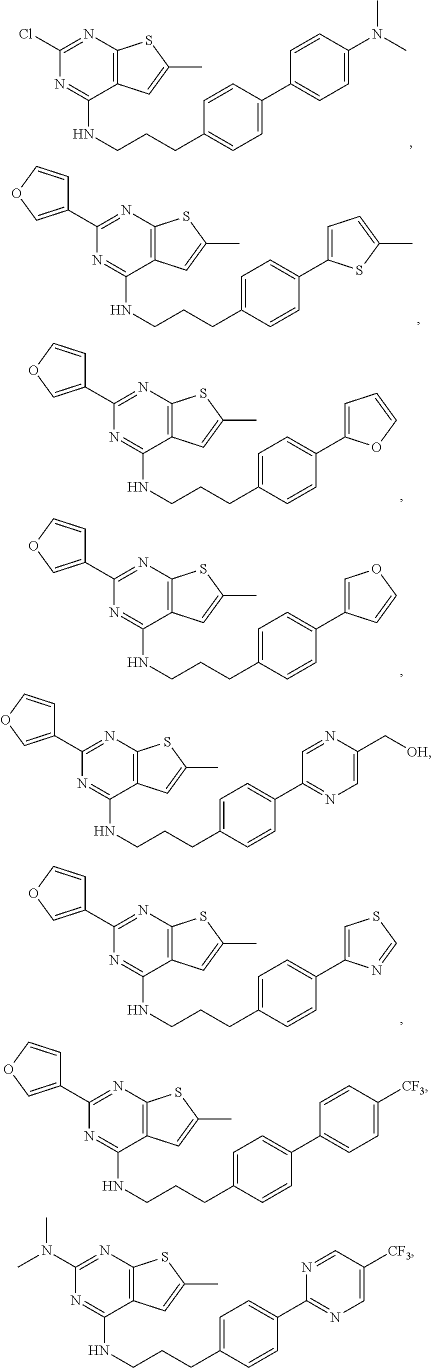

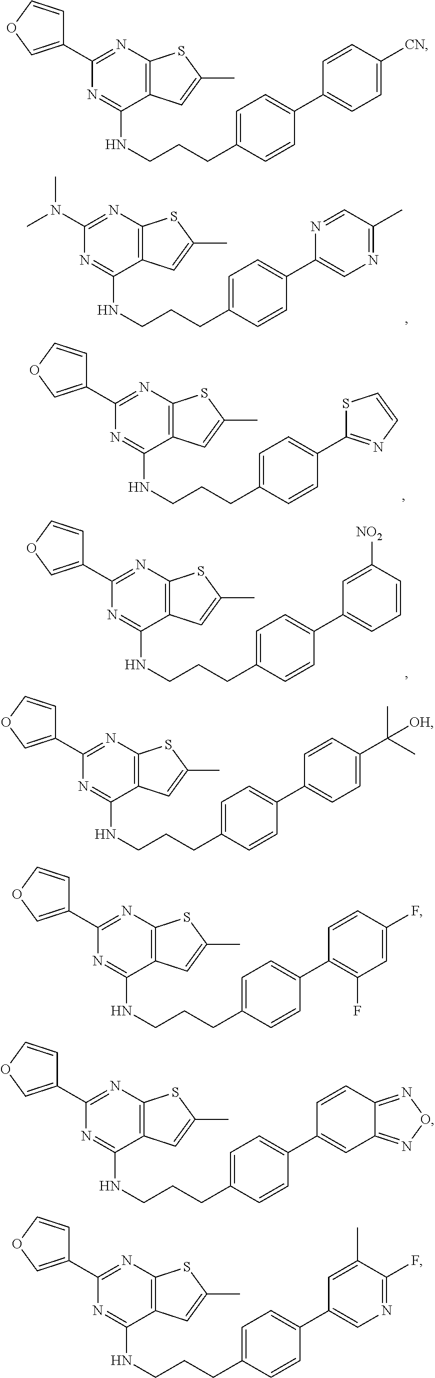

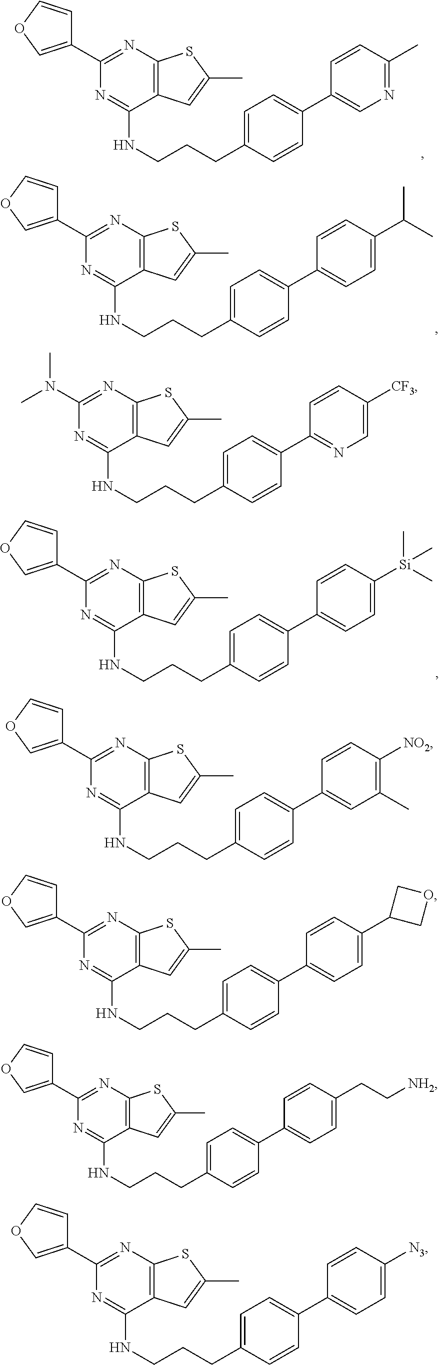

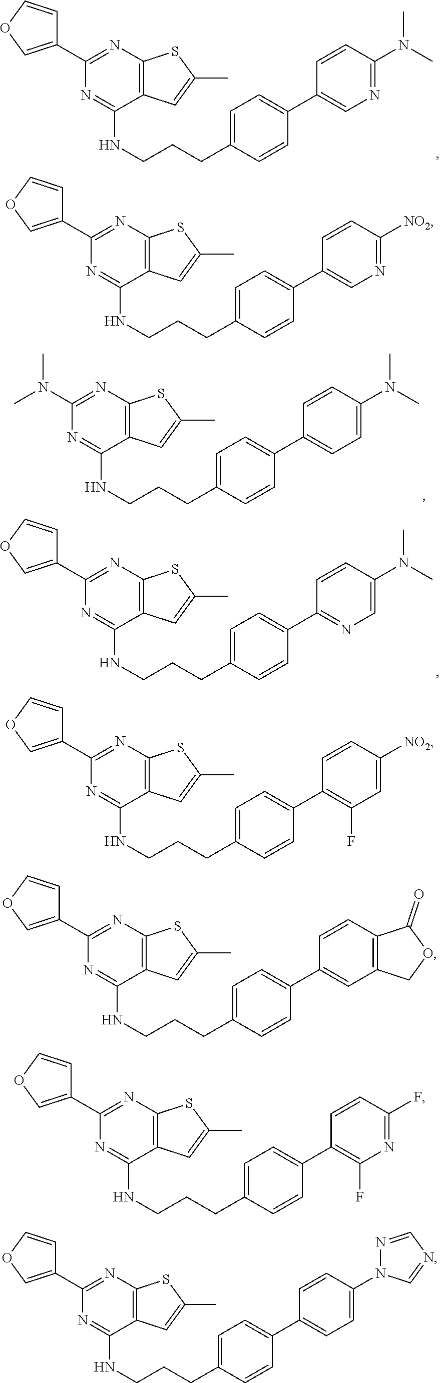

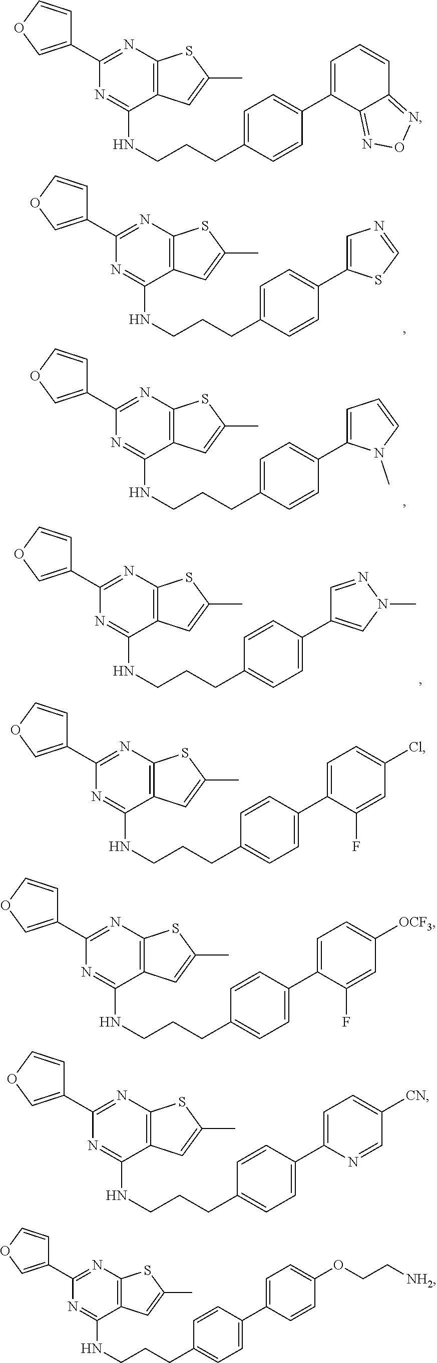

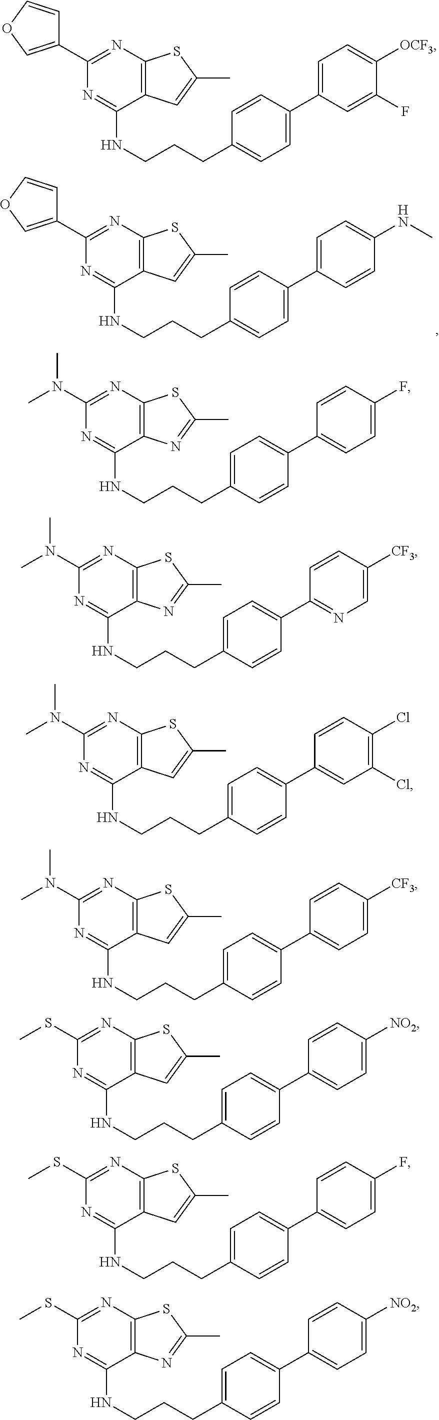

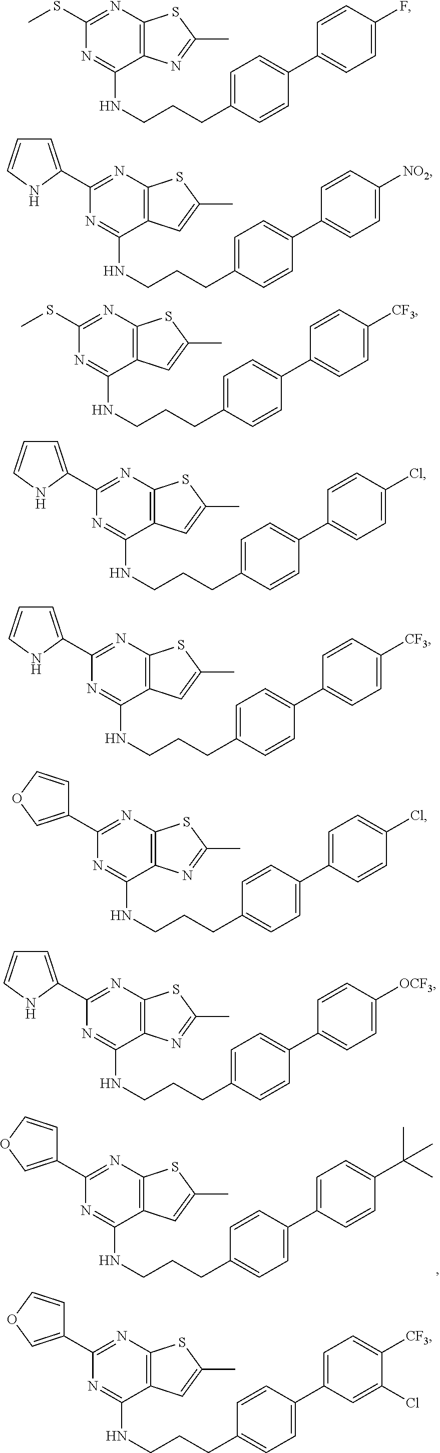

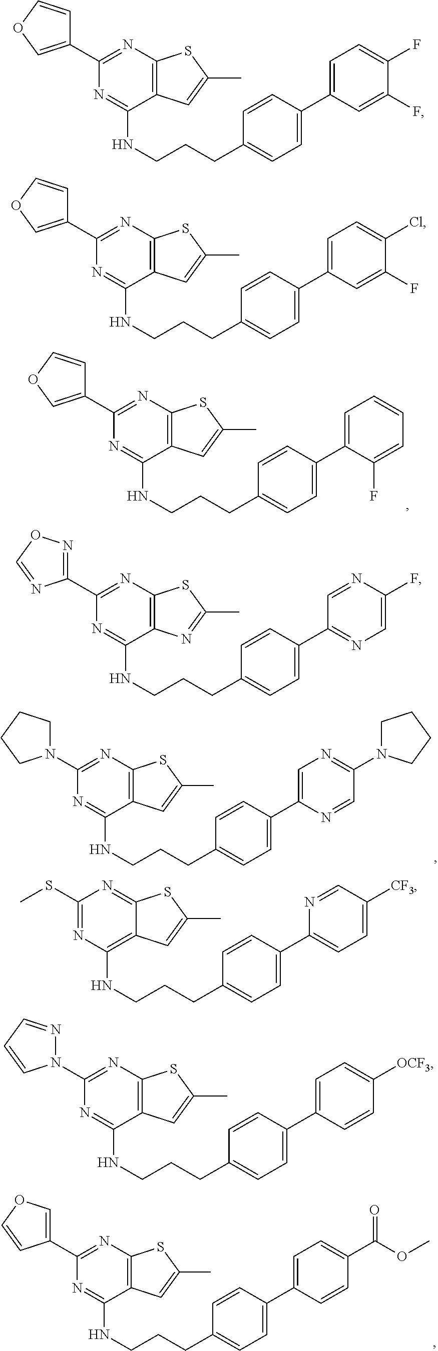

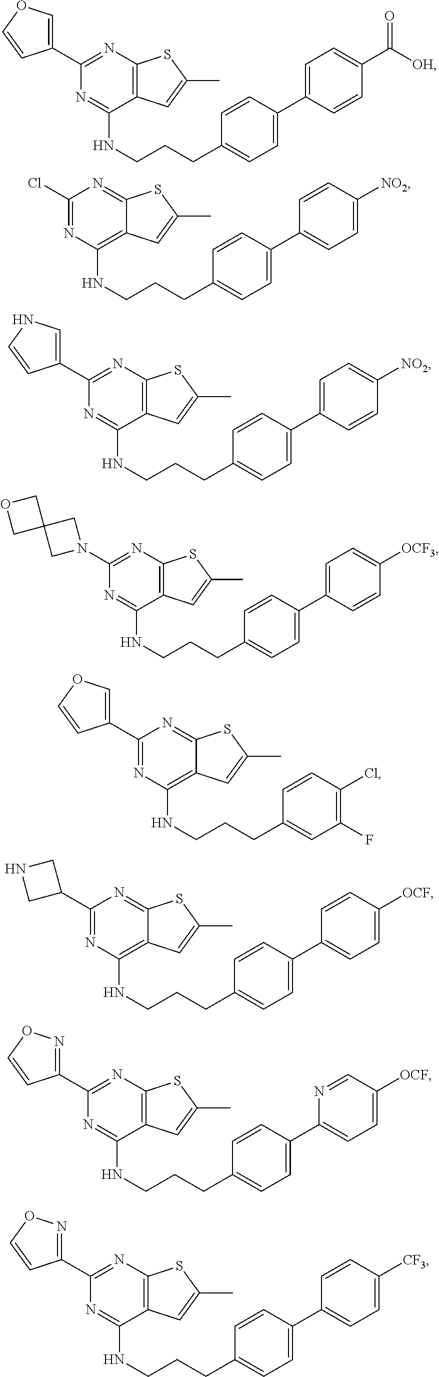

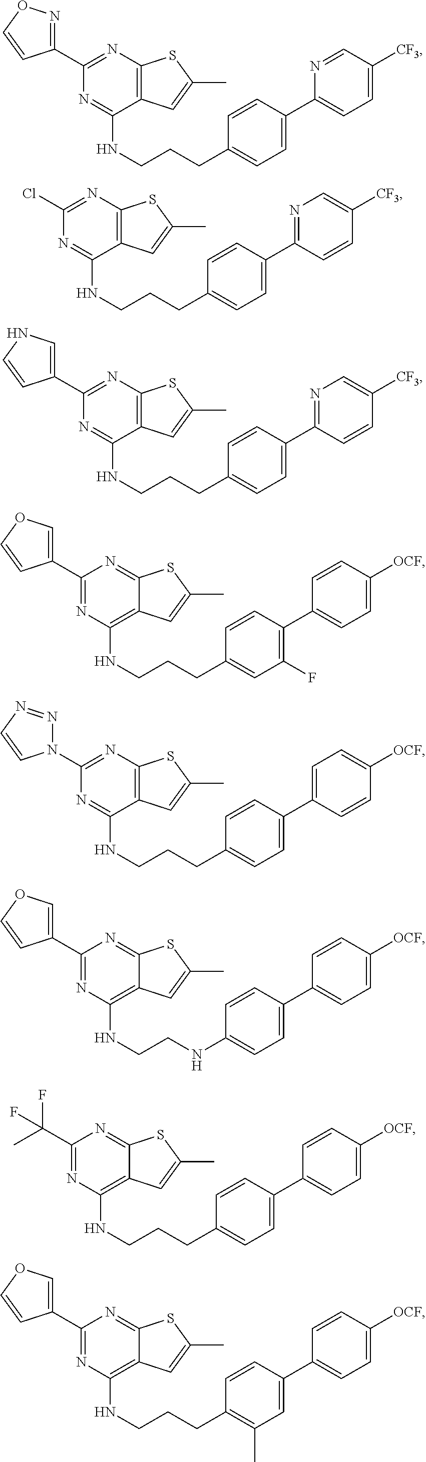

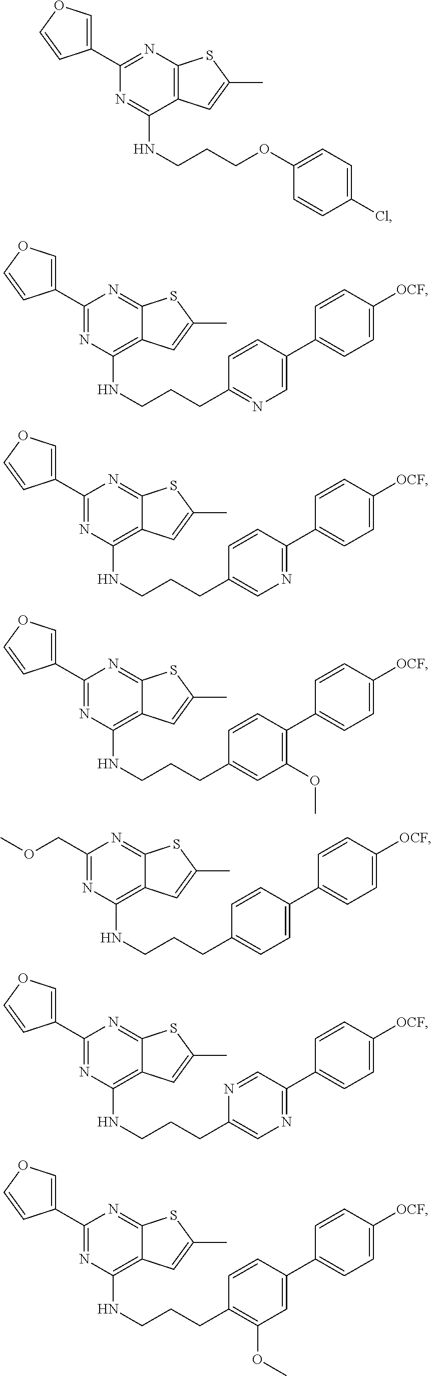

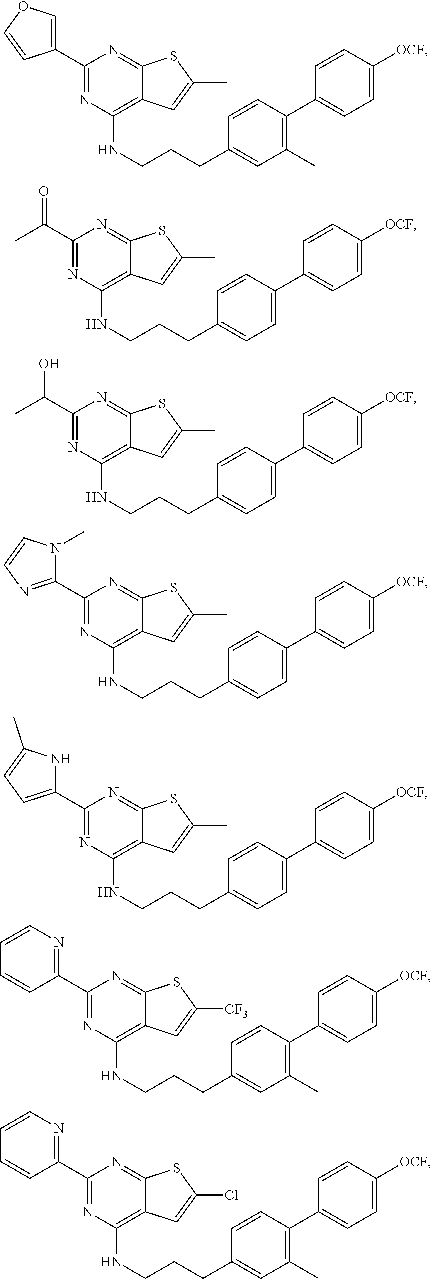

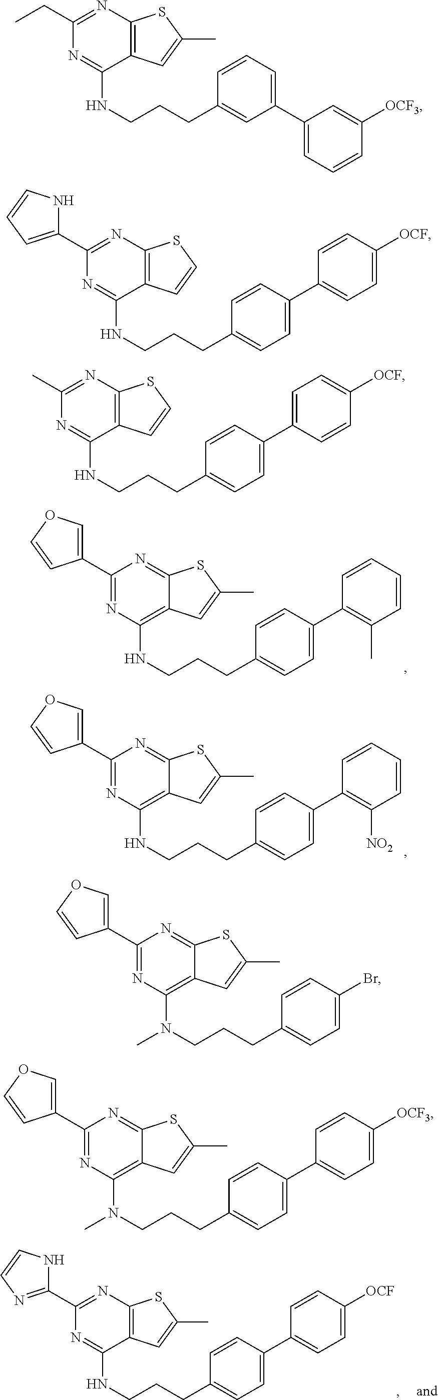

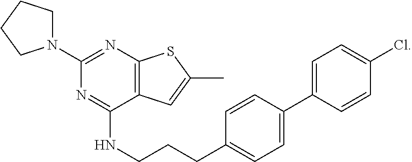 [SMILES: Cc1cc2c(NCCCc3ccc(-c4ccc(Cl)cc4)cc3)nc(N3CCCC3)nc2s1]

39. A pharmaceutical composition comprising a compound of claim 30.

40. The pharmaceutical composition of claim 39, further comprising an excipient.

41. The pharmaceutical composition of claim 40, wherein the pharmaceutical composition is formulated for administration: orally, intra-adiposally, intra-arterially, intra-articularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularly, intravitreally, liposomally, locally, mucosally, parenterally, rectally, subconjunctivally, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion.

42. A method of inhibiting QcrB, a subunit of the cytochrome bc₁-aa₃ supercomplex in Mtb, in a subject in need thereof comprising administering to the subject a therapeutically effective amount of a compound of claim 30.

43. A method of treating tuberculosis in a subject in need thereof comprising administering to the subject a therapeutically effective amount of a compound of claim 30.

44. The method of claim 43 wherein the tuberculosis is caused by a multi-drug resistant mycobacteria and/or an extensively drug resistant mycobacteria.

45.-46. (canceled)

47. The method of claim 43, wherein the method further comprises administering an additional anti-tuberculosis therapy.

48. The method of claim 47, wherein the additional anti-tuberculosis therapy comprises at least one drug selected from the group consisting of: ethambutol, isoniazid, pyrazinamide, rifampicin, streptomycin, an aminoglycoside, a polypeptide antibiotic, a fluoroquinolone, a thioamide, cycloserine, terizidone, rifabutin, a macrolide, linezolid, thioacetazone, thioridazine, arginine, vitamin D, and bedaquiline, and a combination thereof.

49.-53. (canceled)

54. A method of inhibiting the replication of a Mycobacterium tuberculosis bacterium and/or inducing the death of a Mycobacterium tuberculosis bacterium comprising contacting the bacteria with an effective amount of a compound of claim 30.
